(19) United States
(12) Patent Application Publication
(10) Pub. No.: US 2024/0158370 A1
(43) Pub. Date: May 16, 2024

(54) CK1 alpha AND DUAL CK1 alpha / GSPT1 DEGRADING COMPOUNDS

(71) Applicant: Innovo Therapeutics, Inc., San Diego, CA (US)

(72) Inventors: Patrick W. Papa, Carlsbad, CA (US); Veronique Plantevin-Krenitsky, San Francisco, CA (US); Paul J. Krenitsky, San Francisco, CA (US); Frank Mercurio, Rancho Santa Fe, CA (US); Derek Mendy, San Marcos, CA (US); Michael P. Haughey, San Diego, CA (US); Weilin Xie, Rancho Santa Fe, CA (US); Jan Elsner, San Diego, CA (US); John Sapienza, Chula Vista, CA (US)

(21) Appl. No.: 18/462,129

(22) Filed: Sep. 6, 2023

Related U.S. Application Data
(60) Provisional application No. 63/375,167, filed on Sep. 9, 2022.

(51) Int. Cl. C07D 401/14

(52) U.S. Cl. CPC C07D 401/14

(57) ABSTRACT

Provided herein are compounds that act as molecular glues, inducing degradation of CK1α or CK1α/GSPT1, and pharmaceutically acceptable derivatives thereof. Also provided are pharmaceutical compositions containing the compounds and methods of using the compounds for treating a subject with a proliferative disease.

### RELATED APPLICATION

This application claims priority to U.S. Provisional Patent Application No. 63/375,167, filed Sep. 9, 2022, the contents of which are incorporated by reference herein in their entirety.

### FIELD

Provided herein are compounds and compositions for degrading CK1α protein or for degrading both CK1α and GSPT1 proteins. The compounds and compositions are useful in treatment of proliferative diseases, including cancer and autoimmune disorders.

### BACKGROUND

Recently, extensive research has been directed at the discovery of compounds that bind two different proteins, without necessarily inhibiting the function of either protein. Such compounds have been dubbed “molecular glues.” Of particular interest are molecular glues that, upon contact with their targeted proteins, result in degradation of one of the proteins.

This strategy has proven useful for modulation of the activity of proteins that to date have been deemed “undruggable.”

For example, certain molecular glues bind to an E3 ubiquitin ligase. E3 ubiquitin ligases specifically ubiquinate a substrate protein which is then degraded by the proteasome. Cereblon is a key component of one E3 ubiquitin ligase complex and is thus an attractive target for molecular glues. Cereblon is reprogrammed by compounds such as thalidomide, lenalidomide and pomalidomide (imids) to induce degradation of neosubstrate proteins, including IKZF1 (Ikaros) and IKAF3 (Aiolos) (see, e.g., Charlinski et al. Cancers, 2021, 13, 4666). Thus, molecular glues that bind cereblon allow for ubiquination of target proteins, which are then degraded by the proteasome. There has been extensive research in the field of cereblon binding compounds, with many such compounds having been discovered (see, e.g., WO 2022/066835, WO 2020/118098, WO 2021/041664, WO 2019/078522, WO 2021/188537, WO 2021/105334, WO 2022/144416, WO 2021/143816, WO 2022/017365, WO 2022/146151, WO 2022/148358, WO 2021/147889, WO 2021/143822, WO 2020/181232, WO 2019/043214, WO 2020/263832, WO 2020/006233, WO 2015/200795, WO 2019/043217, WO 2019/204354, U.S. Patent Publication Nos. US 2022/0062248, US 2019/0017998, 2020/0206201, 2020/0155690, 2021/0009559, 2018/0215731, 2021/0177825, 2019/0076541, 2021/0403454, 2021/0284624, 2021/0032245, 2020/0207764, 2022/0112211, 2019/0233433, 2020/0207733).

Casein kinase 1α (“CK1α”) is a protein of the CK1 protein family that regulates signaling pathways related to membrane trafficking, cell cycle progression, chromosome segregation, apoptosis, autophagy, cell metabolism, and differentiation in development, circadian rhythm, and the immune response as well as neurodegeneration and cancer (see, e.g., Jiang et al., Cell Commun. Signaling 2018, 16, 23; Spinello et al., Int. J. Mol. Sci. 2021, 22, 3716). Thus, CK1α is an attractive therapeutic target for a variety of indications and uses, including oncology, immuno-oncology, and autoimmune disorders. Mechanistically, CK1α is required for BCR- (via BTK) and TCR-induced activation of the Card11/BCL10/MALT1 (CBM) complex (see, e.g., Gehring et al., Cell Reports 2019, 29, 873-888; Bidere et al., Nature 2009, 458, 7234; Yin et al. Cell. Mol. Life Sci. 2022, 79, 112). Activation of CBM has been implicated in progression of a variety of lymphoid malignancies, including non-Hodgkin lymphoma (NHL) (see, e.g., Bedsaul et al. Front. Onc. 2018, 9, Article 2105), diffuse large B-cell lymphoma (DLBCL) including ABC DLBCL (see, e.g., Thys et al., Front. Onc. 2018, 8, Article 498; Bidere et al., Nature 2009, 458(7234), 92-96), mucosa-associated lymphoid tissue (MALT) lymphomas, mantle cell lymphoma (MCL), adult T-cell leukemia/lymphoma (ATLL) and Sezary syndrome (see, e.g., Juilland et al., Curr. Opin. Hemat. 2016, 23(4), 402-409). Specifically, CK1α has been shown to sustain B-cell signaling in MCL (see, e.g., Manni et al., Front. Oncol. 2021, 11, Article 733848), while MALT1 inhibition has been shown to be an effective strategy in treatment of both naïve and ibrutinib-resistant chronic lymphocytic leukemia (CLL) (see, e.g., Saba et al., Cancer Res. 2017, 77(24), 7038-7048). In immuno-oncology, regulation of the CBM complex has been shown to cause regulatory T-cells to prime tumors for immune checkpoint therapy (see, e.g., Di Pilato et al., Nature 2019, 570(7759), 112-116), while MALT1 activity has been implicated in T-cell immunosuppression (see, e.g., Rosenbaum et al., Nat. Commun. 2019, 10(1), 2352). Inhibition of MALT1 has also been shown to ameliorate autoimmune pathogenesis (see, e.g., Biswas et al., Frontiers in Immunology 2022, 13, 875320).

Loss of CK1α by siRNA or a kinase inhibitor has also been shown to result in stabilization of the tumor suppressor p53 and inhibition of cell cycle progression (see, e.g., Huart et al., J. Biol. Chem. 2009, 284(47), 32384-32394). Briefly, CK1α binds MDM2, which is the p53 E3 ubiquitin ligase (see, e.g., Wu et al. Mol. Cell. Biol. 2012, 32(23), 4821-4832). Binding of the CK1α-MDM2 active complex to p53 promotes degradation of p53 which prevents expression of the cell cycle progression inhibitor p21 (see, e.g., Kocik et al., Cancers 2019, 11, 1014). Thus, degradation of CK1α stabilizes p53 and induces growth arrest (see, e.g., Huart et al., PLoS One 2012, 7(8), e43391). Elevation of p53 activity has been shown to have an antiproliferative and proapoptotic effect in MCL (see, e.g., Tabe et al., Clin. Cancer Res. 2009, 15(3), 933-942; Liang et al., Mod. Pathol. 2010, 23(3), 389-91).

GSPT1 is a translation termination factor that is currently being explored as a therapeutic target for the treatment of acute myeloid leukemia (AML). Recent studies have identified molecular glues that degrade GSPT1 without degrading CK1α (see, e.g., Powell et al., ACS Chem. Biol. 2020, 15, 2722-2730) or that degrade GSPT1 without degrading IKZF1 (Ikaros) (see, e.g., Nishiguchi et al., J. Med. Chem. 2021, 64, 7296-7311).

Thus, there is a need for molecular glues that degrade CK1α or CK1α/GSPT1. Such molecular glues provide therapeutic options for treatment of a variety of proliferative diseases, including cancer and autoimmune diseases

### SUMMARY

Provided herein are compounds and compositions that degrade CK1α or CK1α/GSPT1. In one embodiment, the compounds are molecular glues that bind an E3 ubiquitin ligase and CK1α. In another embodiment, the compounds are molecular glues that bind cereblon and CK1α.

In one embodiment, the compounds for use in the compositions and methods provided herein have Formula I or II:

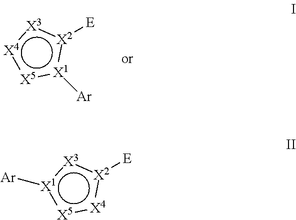

wherein the variables Ar, E and X¹-X⁵ are as defined elsewhere herein.

In another embodiment, provided are pharmaceutical compositions containing a compound provided herein and a pharmaceutically acceptable carrier.

In another embodiment, provided are methods of degrading CK1α or CK1α/GSPT1 using a compound or composition provided herein. The methods provided herein include methods of treatment of CK1α or CK1α/GSPT1 mediated diseases. In one embodiment, the CK1α disease is a B-cell lymphoma or a BTK inhibitor resistant cancer. In another embodiment, the CK1α/GSPT1 disease is AML or breast cancer. In another embodiment, the CK1α degraders provided herein are used in combination with a checkpoint inhibitor, including a CTLA-4, PD-1 or PD-L1 inhibitor, such as anti-CTLA-4, anti-PD-1 or anti-PD-L1 antibodies, in the treatment of cancer.

### DETAILED DESCRIPTION

### I. Definitions

To facilitate understanding of the disclosure set forth herein, a number of terms are defined below.

Unless defined otherwise, all technical and scientific terms used herein have the same meaning as is commonly understood by one of ordinary skill in the art. All patents, applications, published applications and other publications are incorporated by reference in their entirety. In the event that there are a plurality of definitions for a term herein, those in this section prevail unless stated otherwise.

The singular forms “a,” “an,” and “the” include plural references, unless the context clearly dictates otherwise.

As used herein “subject” is an animal, such as a mammal, including human, such as a patient.

As used herein, biological activity refers to the in vivo activities of a compound or physiological responses that result upon in vivo administration of a compound, composition or other mixture. Biological activity, thus, encompasses therapeutic effects and pharmacokinetic behavior of such compounds, compositions and mixtures. Biological activities can be observed in in vitro systems designed to test for such activities.

As used herein, pharmaceutically acceptable derivatives of a compound include, but are not limited to, salts, esters, enol ethers, enol esters, acetals, ketals, orthoesters, hemiacetals, hemiketals, acids, bases, clathrates, solvates or hydrates thereof. Such derivatives may be readily prepared by those of skill in this art using known methods for such derivatization. The compounds produced may be administered to animals or humans without substantial toxic effects and either are pharmaceutically active or are prodrugs. Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethylbenzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and inorganic salts, such as but not limited to, sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates, mesylates, and fumarates. Pharmaceutically acceptable esters include, but are not limited to, alkyl, alkenyl, alkynyl, aryl, aralkyl, and cycloalkyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is alkyl, alkenyl, alkynyl, aryl, aralkyl and cycloalkyl. Pharmaceutically acceptable enol esters include, but are not limited to, derivatives of formula C═C(OC(O)R) where R is hydrogen, alkyl, alkenyl, alkynyl, aryl, aralkyl and cycloalkyl. Pharmaceutically acceptable solvates and hydrates are complexes of a compound with one or more solvent or water molecules, or 1 to about 100, or 1 to about 10, or one to about 2, 3 or 4, solvent or water molecules.

As used herein, treatment means any manner in which one or more of the symptoms of a disease or disorder are ameliorated or otherwise beneficially altered. Treatment also encompasses any pharmaceutical use of the compositions herein, such as use for treating CK1α or CK1α/GSPT1 mediated diseases.

As used herein, amelioration of the symptoms of a particular disorder by administration of a particular compound or pharmaceutical composition refers to any lessening, whether permanent or temporary, lasting or transient that can be attributed to or associated with administration of the compound or pharmaceutical composition.

As used herein, and unless otherwise indicated, the terms “manage,” “managing” and “management” encompass preventing the recurrence of the specified disease or disorder in a subject who has already suffered from the disease or disorder, and/or lengthening the time that a subject who has suffered from the disease or disorder remains in remission. The terms encompass modulating the threshold, development and/or duration of the disease or disorder, or changing the way that a subject responds to the disease or disorder.

As used herein, the DC₅₀ refers to an amount, concentration or dosage of a particular test compound that achieves 50% a maximal response in an assay that measures such response.

Where moieties are specified by their conventional chemical formulae, written from left to right, they equally encompass the chemically identical moieties that would result from writing the structure from right to left, e.g., —CH₂O— is equivalent to —OCH₂—.

The term “alkyl,” by itself or as part of another substituent, means, unless otherwise stated, a straight (i.e., unbranched) or branched chain saturated hydrocarbon radical, which can include di- and multivalent radicals, having the number of carbon atoms designated (i.e., C₁-C₁₀ means one to ten carbons). Examples of alkyl groups include, but are not limited to, groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like.

The term “alkenyl,” by itself or as part of another substituent, means, unless otherwise stated, a straight (i.e., unbranched) or branched chain hydrocarbon radical having one or more carbon-carbon double bonds, which can include di- and multivalent radicals, having the number of carbon atoms designated (i.e., C₁-C₁₀ means one to ten carbons). Examples of alkenyl groups include, but are not limited to, vinyl (i.e., ethenyl), 2-propenyl, crotyl, 2-isopentenyl, 2-(butadienyl), 2,4-pentadienyl, 3-(1,4-pentadienyl), and the higher homologs and isomers.

The term “alkynyl,” by itself or as part of another substituent, means, unless otherwise stated, a straight (i.e., unbranched) or branched chain hydrocarbon radical having one or more carbon-carbon triple bonds, which can include di- and multivalent radicals, having the number of carbon atoms designated (i.e., C₁-C₁₀ means one to ten carbons).

Examples of alkynyl groups include, but are not limited to, ethynyl, 1- and 3-propynyl, 3-butynyl, and the higher homologs and isomers.

The term “alkylene” by itself or as part of another substituent means a divalent radical derived from an alkyl, as exemplified, but not limited, by —CH₂CH₂CH₂CH₂—. Typically, an alkyl (or alkylene) group will have from 1 to 24 carbon atoms, including those groups having 10 or fewer carbon atoms. A “lower alkyl” or “lower alkylene” is a shorter chain alkyl or alkylene group, generally having six or fewer carbon atoms.

The terms “alkoxy,” “alkylamino,” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a straight or branched chain hydrocarbon radical, consisting of a heteroatom selected from the group consisting of O, N, P, Si and S, and wherein the nitrogen and sulfur atoms may optionally be oxidized and the nitrogen atom may have an alkyl substituent to fulfill valency and/or may optionally be quaternized. The heteroatom(s) O, N, P, Si and S may be placed at any interior position of the heteroalkyl group. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For alkylene and heteroalkylene linking groups, no orientation of the linking group is implied by the direction in which the formula of the linking group is written. For example, the formula —C(O)₂R′— represents both —C(O)₂R′- and —R′C(O)₂—.

The terms “cycloalkyl” and “heterocycloalkyl”, by themselves or in combination with other terms, represent, unless otherwise stated, cyclic versions of “alkyl” and “heteroalkyl”, respectively, including bicyclic, tricyclic and bridged bicyclic groups. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, norbornanyl, bicyclo[2.2.2]octanyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, 1- or 2-azabicyclo[2.2.2]octanyl, and the like.

The terms “halo,” by itself or as part of another substituent, means, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is meant to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent which can be a single ring or multiple rings (in one embodiment from 1 to 3 rings) which are fused together or linked covalently. The term “heteroaryl” refers to aryl groups that contain from one to four heteroatoms selected from N, O, and S in the ring(s), wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituent moieties for aryl and heteroaryl ring systems may be selected from the group of acceptable substituent moieties described herein.

The term “heteroarylium” refers to a heteroaryl group that is positively charged on one or more of the heteroatoms.

The term “oxo” as used herein means an oxygen atom that is double bonded to a carbon atom.

Each of the above terms (e.g., “alkyl,” “heteroalkyl,” “aryl” and “heteroaryl”) are meant to include both substituted and unsubstituted forms of the indicated radical. Non-limiting examples of substituent moieties for each type of radical are provided below.

Substituent moieties for alkyl, heteroalkyl, alkylene, alkenyl, heteroalkylene, heteroalkenyl, alkynyl, cycloalkyl, heterocycloalkyl, cycloalkenyl, and heterocycloalkenyl groups are, in one embodiment, selected from, deuterium, —OR′, ═O, ═NR′, ═N—OR′, —NR′R″, —SR′, halo, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NRSO₂NR′R″, —CN and —NO₂ in a number ranging from zero to the number of hydrogen atoms in such radical. In one embodiment, substituent moieties for cycloalkyl, heterocycloalkyl, cycloalkenyl, and heterocycloalkenyl groups also include substituted and unsubstituted alkyl, substituted and unsubstituted alkenyl, and substituted and unsubstituted alkynyl. R′, R″, R′″ and R″″ each in one embodiment independently are hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. When a compound provided herein includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituent moieties, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Substituent moieties for aryl and heteroaryl groups are, in one embodiment, selected from deuterium, halo, substituted and unsubstituted alkyl, substituted and unsubstituted alkenyl, and substituted and unsubstituted alkynyl, —OR′, —NR′R″, —SR′, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of hydrogens on the aromatic ring system; and where R′, R″, R′″ and R″″ are, in one embodiment, independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound provided herein includes more than one R group, for example, each of the R groups is independently selected as are each R′, R″, R′″ and R″″ groups when more than one of these groups is present.

Two of the substituent moieties on adjacent atoms of an aryl or heteroaryl ring may optionally form a ring of the formula -Q′-C(O)—(CRR′)_{q}-Q″-, wherein Q′ and Q″ are independently —NR—, —O—, —CRR′— or a single bond, and q is an integer of from 0 to 3. Alternatively, two of the substituent moieties on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituent moieties on adjacent atoms of the aryl or heteroaryl ring may optionally be replaced with a substituent of the formula —(CRR′)ₛ—X′—(CR″R′″)ₐ—, where s and d are independently integers of from 0 to 3, and X′ is —O—, —NR′—, —S—, —S(O)—, —S(O)₂—, or —S(O)₂NR′—. The substituent moieties R, R′, R″ and R′″ are, in one embodiment, independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “heteroatom” or “ring heteroatom” is meant to include oxygen (O), nitrogen (N), sulfur (S), phosphorus (P), and silicon (Si).

As used herein, a prodrug is a compound that upon in vivo administration is metabolized, or otherwise undergoes chemical changes under physiological conditions, by one or more steps or processes or otherwise converted to a biologically, pharmaceutically or therapeutically active form of the compound. Additionally, prodrugs can be converted to a biologically, pharmaceutically or therapeutically active form of the compound by chemical or biochemical methods in an ex vivo environment. For example, prodrugs can be converted to the compounds of the present invention when placed in a transdermal patch reservoir with a suitable enzyme or chemical reagent.

Certain compounds provided herein can exist in unsolvated forms as well as solvated forms, including hydrated forms. In general, the solvated forms are equivalent to unsolvated forms and are encompassed within the scope of the present disclosure. Certain compounds provided herein may exist in multiple crystalline or amorphous forms. In general, all physical forms are equivalent for the uses contemplated herein and are intended to be within the scope of the present disclosure.

Certain compounds provided herein possess asymmetric carbon atoms (optical centers) or double bonds; the racemates, diastereomers, tautomers, geometric isomers and individual isomers are encompassed within the scope of the present disclosure. The compounds provided herein do not include those which are known in the art to be too unstable to synthesize and/or isolate.

The compounds provided herein may also contain unnatural proportions of atomic isotopes at one or more of the atoms that constitute such compounds. For example, the compounds may be radiolabeled with radioactive isotopes, such as for example tritium (H), iodine-125 (¹²⁵I) or carbon-14 (¹⁴C). All isotopic variations of the compounds provided herein, whether radioactive or not, are encompassed within the scope of the present disclosure.

### II. Compounds for Use in Compositions and Methods

In one embodiment, provided herein is a compound for use in the compositions and methods provided herein having Formula I or II:

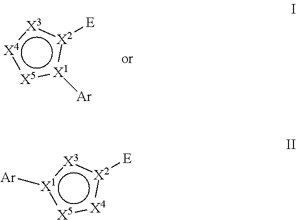

wherein Ar is aryl, heteroaryl, C₅₋₇cycloalkyl, C₅₋₇cycloalkenyl, a 5-7 membered heterocyclyl or a 5-7 membered heterocycloalkenyl; E is a moiety that binds to an E3 ubiquitin ligase; X¹-X² are each independently N or C; and X³-X⁵ are each independently CR, N, NR, S or O, where each R is independently H, alkyl, alkenyl, alkynyl, cycloalkyl or heterocyclyl; or two R groups that are on adjacent positions on the ring together form alkylene; or R and Ar that are on adjacent positions on the 5 membered ring together form a fused ring.

In one embodiment, provided herein is a compound for use in the compositions and methods provided herein having Formula I or II:

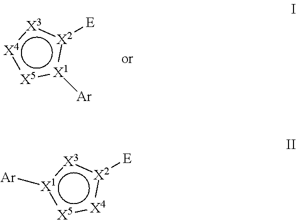

wherein Ar is aryl, heteroaryl, C₅₋₇cycloalkyl or a 5-7 membered heterocyclyl; E is a moiety that binds to an E3 ubiquitin ligase; X¹-X² are each independently N or C; and X³-X⁵ are each independently CR, N, NR, S or O, where each R is independently H, alkyl, alkenyl, alkynyl, heteroalkyl, cycloalkyl, heterocyclyl, aryl or heteroaryl; or two R groups that are on adjacent positions on the ring together form alkylene.

In another embodiment, provided herein is a compound for use in the compositions and methods provided herein having Formula I or II:

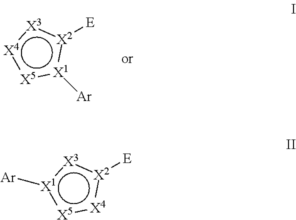

wherein Ar is aryl; E is a moiety that binds to an E3 ubiquitin ligase; X¹-X² are each independently N or C; and X³-X⁵ are each independently CR, N, NR, S or O, where each R is independently H, alkyl, alkenyl, alkynyl, heteroalkyl, cycloalkyl, heterocyclyl, aryl or heteroaryl; or two R groups that are on adjacent positions on the ring together form alkylene.

In another embodiment, X³-X⁵ are each independently CR, N, NR or S. In another embodiment, X³-X⁵ are each independently CR, N or NR.

In another embodiment, the compounds of Formula II are selected with the proviso that when X¹ is C, X², X⁴ and X⁵ are N and X³ is CH, then E is not an isoindolindione moiety. In another embodiment, the compounds of Formula II are selected with the proviso that when X⁴ and X⁵ are N, then X² is not N.

In another embodiment, the compounds of Formula II are selected with the proviso that when X¹ and X² are C, X³ is NMe, X⁴ is N and X⁵ is CH, then E is not an isoindolindione moiety. In another embodiment, the compounds of Formula II are selected with the proviso that when X⁴ is N, then X³ is not NMe. In another embodiment, the compounds of Formula II are selected with the proviso that when X³ is NMe, then X⁴ is not N.

In another embodiment, the compounds of Formula II are selected with the proviso that when X¹ and X² are C, X³ is CH, X⁴ is N and X⁵ is NMe, then Ar is not 5-fluoro-2-pyridyl. In another embodiment, the compounds of Formula II are selected with the proviso that when X¹ and X² are C, X³ is CH, X⁴ is N and X⁵ is NMe, then Ar is not heteroaryl.

In another embodiment, the compounds of Formula I are selected with the proviso that when X¹ is N, X² is C, X³ and X⁴ are CH and X⁵ is N, then Ar is not cyclopropyl. In another embodiment, the compounds of Formula I are selected with the proviso that when X¹ is N, X² is C, X³ and X⁴ are CH and X⁵ is N, then Ar is not cycloalkyl.

In another embodiment, the compounds of Formula II are selected with the proviso that when X¹ is N, X² is C, X³ and X⁴ are CH and X⁵ is N, then Ar is not phenyl. In another embodiment, the compounds of Formula II are selected with the proviso that when X¹ is N, X² is C, X³ and X⁴ are CH and X⁵ is N, then Ar is not aryl.

In another embodiment, the compounds of Formula II are selected with the proviso that when X¹ and X³ are N, X² is C, and X⁴ and X⁵ are CH, then Ar is not phenyl. In another embodiment, the compounds of Formula II are selected with the proviso that when X¹ and X³ are N, X² is C, and X⁴ and X⁵ are CH, then Ar is not aryl.

In another embodiment, the compounds of Formula II are selected with the proviso that the ring containing X¹-X⁵ is not 1,2,3-triazol-1,4-diyl.

In another embodiment, the compounds of Formula I and II are selected with the proviso that Ar is not tetrahydropyran-2-yl. In another embodiment, the compounds of Formula I and II are selected with the proviso that Ar is not tetrahydropyranyl.

In another embodiment, the compound of Formula I is not 3-[1,3-dihydro-1-oxo-5-(5-phenyl-4-oxazolyl)-2H-isoindol-2-yl]-2,6-piperidinedione. In another embodiment, the compound of Formula II is not 3-[1,3-dihydro-1-oxo-5-(2-phenyl-4-oxazolyl)-2H-isoindol-2-yl]-2,6-piperidinedione. In another embodiment, the compound of Formula II is not 3-[1,3-dihydro-1-oxo-5-(3-phenyl-TH-1,2,4-triazol-5-yl)-2H-isoindol-2-yl]-2,6-piperidinedione.

In certain embodiments, the compounds provided herein contain multiple E groups. In another embodiment, the compounds provided herein have one of the following formulae:

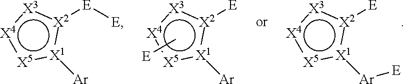

where Ar, E and X¹-X⁵ are as defined elsewhere herein.

In one embodiment, X¹ is C and X² is N. In another embodiment, X¹ is N and X² is C. In another embodiment, X¹ and X² are both C and the compound has the structure:

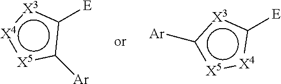

where Ar, E and X³-X⁵ are as defined elsewhere herein.

In another embodiment, the compound provided herein has one of the following formulae:

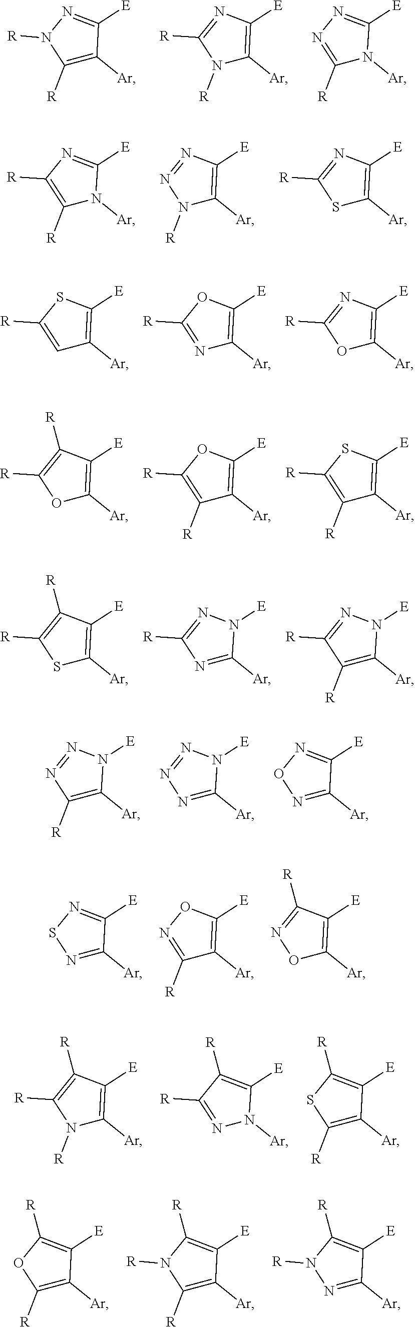

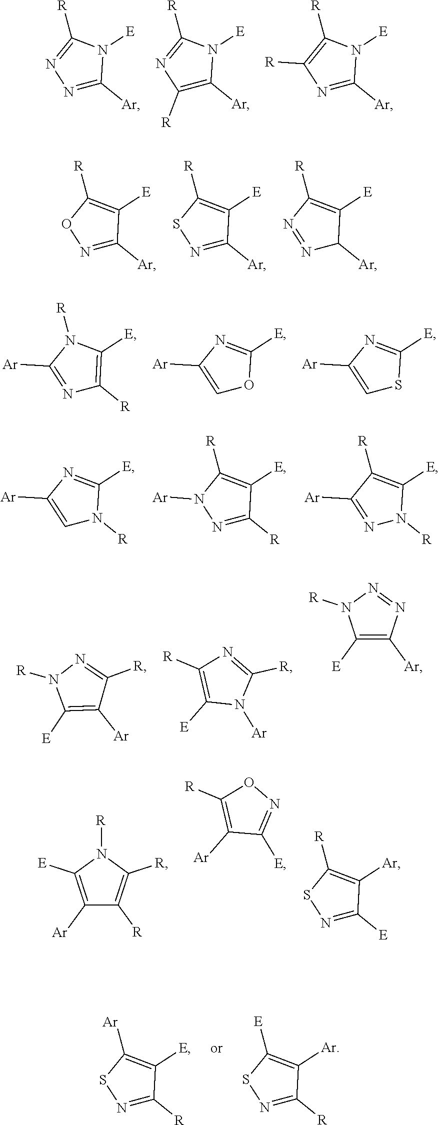

In another embodiment, X¹ and X² are both C, X⁵ is NR, and the compound has the structure:

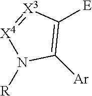

where Ar, E, X³ and X⁴ are as defined elsewhere herein.

In another embodiment, X¹ and X² are both C, X³ is CR, X⁴ is N and X⁵ is NR, and the compound has the structure:

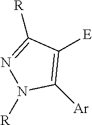

where Ar, E and R are as defined elsewhere herein. In another embodiment, X³ is CH and the compound has the structure:

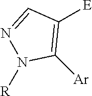

where Ar, E and R are as defined elsewhere herein. In another embodiment, the compound has the structure:

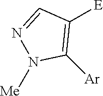

where Ar and E are as defined elsewhere herein.

In another embodiment, X¹ and X² are both C, X³ is N, X⁴ is CR and X⁵ is NR, and the compound has the structure:

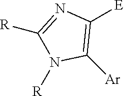

where Ar, E and R are as defined elsewhere herein. In another embodiment, X⁴ is CH and the compound has the structure:

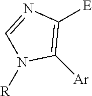

where Ar, E and R are as defined elsewhere herein. In another embodiment, the compound has the structure:

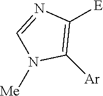

where Ar and E are as defined elsewhere herein.

In another embodiment, the compound has the structure:

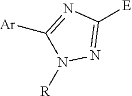

where R, Ar and E are as defined elsewhere herein.

In another embodiment, the compound has the structure:

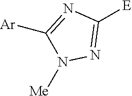

where Ar and E are as defined elsewhere herein.

In another embodiment, the compound has the structure:

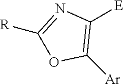

where R, Ar and E are as defined elsewhere herein.

In another embodiment, the compound has the structure:

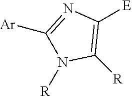

where R, Ar and E are as defined elsewhere herein.

In another embodiment, the compound has the structure:

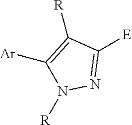

where R, Ar and E are as defined elsewhere herein.

In another embodiment, the compound has the structure:

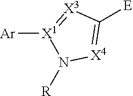

where X¹, X³, X⁴, R, Ar and E are as defined elsewhere herein.

In another embodiment, each R is independently H, alkyl, alkenyl, alkynyl, cycloalkyl or heterocyclyl; or two R groups that are on adjacent positions on the ring together form alkylene. In another embodiment, each R is independently H, alkyl, alkenyl or alkynyl. In another embodiment, each R is independently H, alkyl, cycloalkyl, heterocyclyl or aryl; or two R groups that are on adjacent positions on the ring together form lower alkylene. In another embodiment, each R is independently H, alkyl, cycloalkyl or aryl; or two R groups that are on adjacent positions on the ring together form lower alkylene. In another embodiment, each R is independently H or alkyl; or two R groups that are on adjacent positions on the ring together form lower alkylene. In another embodiment, each R is independently H, alkyl or haloalkyl. In another embodiment, each R is independently, H, methyl, ethyl, isopropyl, difluoromethyl, fluoromethyl, trifluoromethyl, 2,2-difluoro-1-ethyl, 2,2,2-trifluoro-1-ethyl, difluoropropyl, cyclopropyl, trideuteromethyl, ethyl, 2-hydroxy-2-methylpropyl, cyclohexyl, 1,3-dioxanyl, 4-pyranyl or phenyl; or or two R groups that are on adjacent positions on the ring together form propylene. In another embodiment, each R is independently, H, methyl, difluoromethyl, fluoromethyl, trifluoromethyl, 2,2,2-trifluoro-1-ethyl, cyclopropyl, trideuteromethyl, ethyl, 2-hydroxy-2-methylpropyl, cyclohexyl, 4-pyranyl or phenyl; or or two R groups that are on adjacent positions on the ring together form propylene. In another embodiment, each R is independently, H, methyl, difluoromethyl, fluoromethyl, trifluoromethyl, cyclopropyl, trideuteromethyl, ethyl, 2-hydroxy-2-methylpropyl or phenyl; or or two R groups that are on adjacent positions on the ring together form propylene. In another embodiment, each R is independently H, methyl, difluoromethyl or 2,2,2-trifluoro-1-ethyl. In another embodiment, each R is independently H or methyl. In another embodiment, each R is H. In another embodiment, each R is methyl.

In another embodiment, E is a moiety that binds to cereblon. In another embodiment, E contains an imide, amide, thioamide or thioimide derived moiety. In another embodiment, E contains a phthalimido group or an analog or derivative thereof. In another embodiment, E contains a phthalimido-glutarimide group or an analog or derivative thereof. In another embodiment, E contains a thalidomide, lenalidomide or pomalidomide moiety, or an analog or derivative thereof.

In another embodiment, E has one of the following formulae:

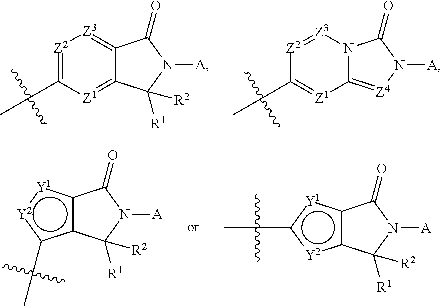

where A is a cyclic amide or cyclic imide or a derivative thereof; R¹ and R² are each independently H, alkyl, alkenyl, alkynyl, cycloalkyl, heterocyclyl, aryl or heteroaryl; one of Y¹ and Y² is S and the other is CR³, where R³ is H, alkyl, alkenyl, alkynyl, cycloalkyl, heterocyclyl, aryl or heteroaryl; and Z¹-Z⁴ are each independently N or CR⁴, where each R⁴ is independently H, alkyl, alkenyl, alkynyl, cycloalkyl, heterocyclyl, aryl or heteroaryl. In one embodiment, at most two of Z¹-Z⁴ are N. In another embodiment, Z¹ and R¹, together with the atoms to which they are attached, form a fused phenyl ring; R² is absent; and E has the formula:

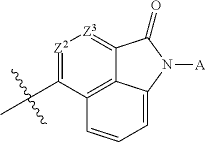

where A is a cyclic amide or cyclic imide or a derivative thereof; and Z² and Z³ are each independently N or CR⁴, where each R⁴ is independently H, alkyl, alkenyl, alkynyl, cycloalkyl, heterocyclyl, aryl or heteroaryl.

In one embodiment, A is a cyclic imide having the structure:

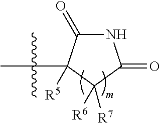

where R⁵ is H or alkyl; R⁶ and R⁷ are each independently H, alkyl, alkenyl, alkynyl, cycloalkyl or heterocyclyl; and m is an integer from 1-4.

In another embodiment, R⁵ is H or lower alkyl. In another embodiment, R⁵ is H or methyl. In another embodiment, R⁵ is H. In another embodiment, R⁵ is methyl.

In another embodiment, R⁶ and R⁷ are each independently H or alkyl. In another embodiment, R⁶ and R⁷ are each independently H or methyl. In another embodiment, R⁶ and R⁷ both H.

In another embodiment, m is 1, 2 or 3. In another embodiment, m is 2 or 3. In another embodiment, m is 2. In another embodiment, m is 3.

In another embodiment, A has the structure:

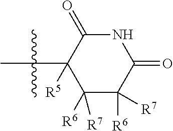

where R⁵-R⁷ are selected as described elsewhere herein.

In another embodiment, A has the structure:

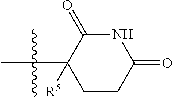

where R⁵ is selected as described elsewhere herein.

In another embodiment, A has one of the following structures with the absolute stereochemistry shown:

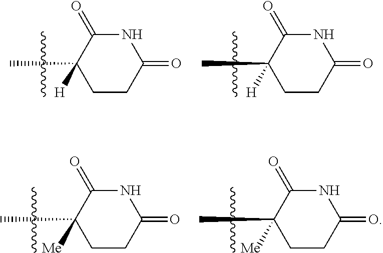

In another embodiment, R¹ and R² are each independently H, alkyl, alkenyl or alkynyl. In another embodiment, R¹ and R² are each independently H or alkyl. In another embodiment, R¹ and R² are each independently H or methyl. In another embodiment, R¹ and R² are each H.

In another embodiment, R³ is H, alkyl, alkenyl or alkynyl. In another embodiment, R³ is H or alkyl. In another embodiment, R³ is H or methyl. In another embodiment, R³ is H.

In another embodiment, R⁴ is H, alkyl, alkenyl or alkynyl. In another embodiment, R⁴ is H or alkyl. In another embodiment, R⁴ is H or methyl. In another embodiment, R⁴ is H.

In another embodiment, Y¹ is S and Y² is CR³. In another embodiment, Y¹ is S and Y² is CH. In another embodiment, Y¹ is CR³ and Y² is S. In another embodiment, Y¹ is CH and Y² is S.

In another embodiment, Z¹ is N and Z²-Z⁴ are CR⁴. In another embodiment, Z¹ is N and Z²-Z⁴ are CH.

In another embodiment, Z² is N and Z¹, Z³ and Z⁴ are CR⁴. In another embodiment, Z² is N and Z¹, Z³ and Z⁴ are CH.

In another embodiment, Z³ is N and Z¹, Z² and Z⁴ are CR⁴. In another embodiment, Z³ is N and Z¹, Z² and Z⁴ are CH.

In another embodiment, Z⁴ is N and Z¹-Z³ are CR⁴. In another embodiment, Z⁴ is N and Z¹-Z³ are CH.

In another embodiment, E is an imid. In another embodiment, E is selected from:

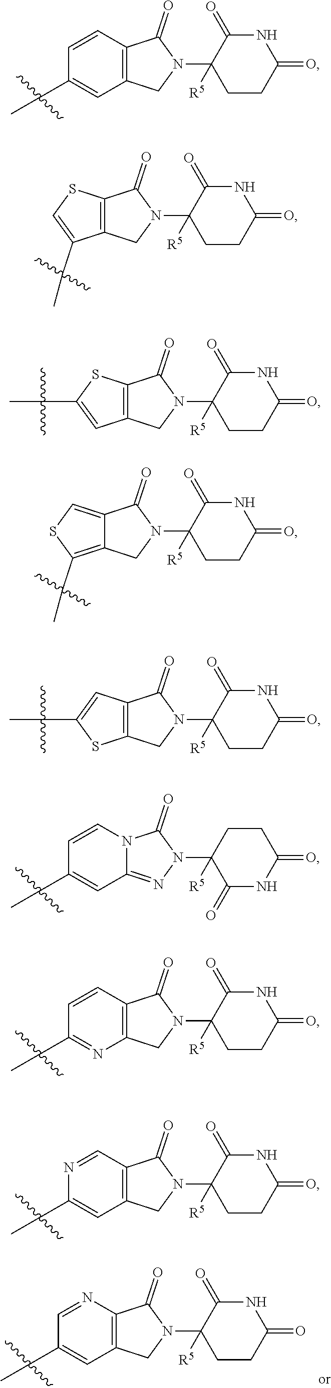

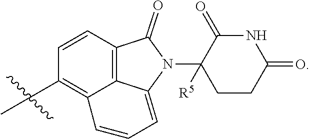

where R⁵ is as defined elsewhere herein.

In another embodiment, E has the structure:

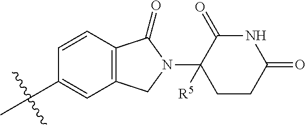

where R⁵ is as defined elsewhere herein. In another embodiment, E is selected from:

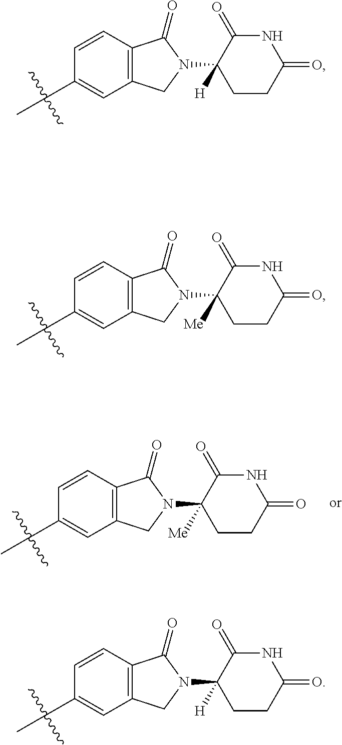 [SMILES: *c1ccc2c(c1)CN([C@H]1CCC(=O)NC1=O)C2=O ; *c1ccc2c(c1)CN([C@@]1(C)CCC(=O)NC1=O)C2=O ; *c1ccc2c(c1)CN([C@]1(C)CCC(=O)NC1=O)C2=O ; *c1ccc2c(c1)CN([C@@H]1CCC(=O)NC1=O)C2=O]

In another embodiment, Ar is aryl, heteroaryl, C₅₋₇cycloalkyl, C₅₋₇cycloalkenyl, a 5-7 membered heterocyclyl with at least one N atom in the ring or a 5-7 membered heterocycloalkenyl with at least one N atom in the ring.

In one embodiment, Ar is optionally substituted phenyl, optionally substituted biphenyl, optionally substituted naphthyl, optionally substituted pyridyl, optionally substituted pyrimidinyl, optionally substituted pyridazinyl, optionally substituted pyrazolyl, optionally substituted pyridopyrazolyl, optionally substituted isoxazolyl, optionally substituted indolyl, optionally substituted isoindolyl, optionally substituted thienyl, optionally substituted benzofuryl, optionally substituted imidazopyridyl, optionally substituted benzopyrazolyl, optionally substituted pyrrolopyridyl, optionally substituted benzimidazolyl, optionally substituted benzothiazolyl, optionally substituted thienopyridyl, optionally substituted dihydrobenzofuryl, optionally substituted benzopyridazinyl, optionally substituted benzopyranyl, optionally substituted benzothienyl, optionally substituted triazolopyrimidinyl, optionally substituted piperidinyl, optionally substituted cyclohexenyl, optionally substituted tetrahydropyridyl, optionally substituted tetrahydrofuranyl, optionally substituted dihydrofuranyl, optionally substituted morpholinyl, optionally substituted tetrahydroisoquinolinyl, optionally substituted azepinyl, optionally substituted isoquinolinyl, optionally substitutedcycloheptenyl, optionally substituted indenyl, optionally substituted dihydronaphthyl, optionally substituted 8-azabicyclooctanyl, optionally substituted adamantanyl, optionally substituted dihydroindenyl, optionally substituted cyclopropyl or optionally substituted cyclohexyl.

In one embodiment, Ar is optionally substituted phenyl, optionally substituted biphenyl, optionally substituted naphthyl, optionally substituted pyridinyl, optionally substituted pyrimidinyl, optionally substituted pyrazolyl, optionally substituted pyridopyrazolyl, optionally substituted isoxazolyl, optionally substituted indolyl, optionally substituted isoindolyl, optionally substituted thienyl, optionally substituted dihydrobenzofuryl, optionally substituted dihydroindenyl, optionally substituted cyclopropyl or optionally substituted cyclohexyl. In another embodiment, Ar is optionally substituted phenyl, optionally substituted biphenyl or optionally substituted naphthyl.

In another embodiment, Ar is phenyl, biphenyl, naphthyl, pyridinyl, pyrimidinyl, pyrazolyl, pyridopyrazolyl, isoxazolyl, indolyl, isoindolyl, thienyl, dihydrobenzofuyl, dihydroindenyl, cyclopropyl or cyclohexyl, each optionally substituted with one or more substituents each independently selected from halo, cyano, alkyl, alkenyl, alkynyl, haloalkyl, cycloalkyl, heterocyclyl, heteroaryl, cycloalkylalkyl, heterocyclylalkyl, COR⁸, OR⁹, NR¹⁰R¹¹ and S(O)ₙR¹², where:

R⁸ is alkyl, OR¹³ or NR¹⁴R¹⁵;
R⁹ is H, alkyl, cycloalkyl, heterocyclyl, aryl, heteroaryl or COR¹⁶;
R¹⁰ and R¹¹ are each independently H, alkyl, cycloalkyl, heterocyclyl, aryl, heteroaryl or COR¹⁷;
R¹² is alkyl, cycloalkyl, heterocyclyl, aryl, heteroaryl, OR¹⁸ or NR¹⁴R¹⁵;
each R¹³, R¹⁴ and R¹⁵ are each independently H, alkyl, cycloalkyl, heterocyclyl, aryl or heteroaryl;
R¹⁶ is alkyl, cycloalkyl, heterocyclyl, aryl, heteroaryl, OR¹³ or NR¹⁴R¹;
R¹⁷ is alkyl, cycloalkyl, heterocyclyl, aryl, heteroaryl, OR¹³ or NR¹⁴R¹;
R¹⁸ is alkyl, cycloalkyl, heterocyclyl, aryl or heteroaryl; and
n is 0, 1 or 2.

In another embodiment, Ar is phenyl, biphenyl or naphthyl, each optionally substituted with one or more substituents each independently selected from halo, cyano, alkyl, alkenyl, alkynyl, haloalkyl, cycloalkyl, heterocyclyl, heteroaryl, cycloalkylalkyl, heterocyclylalkyl, COR⁸, OR⁹, NR¹⁰R¹¹ and S(O)ₙR¹², where:

R⁸ is alkyl, OR¹³ or NR¹⁴R¹⁵;
R⁹ is H, alkyl, cycloalkyl, heterocyclyl, aryl, heteroaryl or COR¹⁶;
R¹⁰ and R¹¹ are each independently H, alkyl, cycloalkyl, heterocyclyl, aryl, heteroaryl or COR¹⁷;
R¹² is alkyl, cycloalkyl, heterocyclyl, aryl, heteroaryl, OR¹⁸ or NR¹⁴R¹⁵; each R¹³, R¹⁴ and R¹⁵ are each independently H, alkyl, cycloalkyl, heterocyclyl, aryl or heteroaryl;
R¹⁶ is alkyl, cycloalkyl, heterocyclyl, aryl, heteroaryl, OR¹³ or NR¹⁴R¹⁵;
R¹⁷ is alkyl, cycloalkyl, heterocyclyl, aryl, heteroaryl, OR¹³ or NR¹⁴R¹⁵;
R¹⁸ is alkyl, cycloalkyl, heterocyclyl, aryl or heteroaryl; and
n is 0, 1 or 2.

In one embodiment, Ar is substituted with 1 to 5, or from 1 to 3, or 1 or 2 substituents. In another embodiment, Ar is unsubstituted.

In another embodiment, Ar is phenyl, optionally substituted with one or more substituents each independently selected from halo, cyano, alkyl, alkenyl, alkynyl, haloalkyl, cycloalkyl, heterocyclyl, aryl, heteroaryl, cycloalkylalkyl, heterocyclylalkyl, COR⁸, OR⁹, NR¹⁰R¹¹ and S(O)ₙR¹². In another embodiment, Ar is phenyl, optionally substituted with one or more substituents each independently selected from halo, cyano, alkyl, haloalkyl, cycloalkyl, heterocyclyl, heteroaryl, heterocyclylalkyl, COR⁸, OR⁹, NR¹⁰R¹¹ and S(O)ₙR¹². In another embodiment, Ar is phenyl, optionally substituted with one or more substituents each independently selected from halo, cyano, alkyl, haloalkyl, cycloalkyl, heterocyclyl, heteroaryl, heterocyclylalkyl, CONR¹⁴R¹⁵, OR⁹, NR¹⁰R¹¹ and S(O)₂R¹².

In another embodiment, Ar is phenyl, optionally substituted with one or more substituents each independently selected from chloro, fluoro, cyano, methyl, isopropyl, isobutyl, tert-butyl, trifluoromethyl, difluoromethyl, hydroxymethyl, methoxymethyl, phenoxymethyl, dimethylaminomethyl, cyclopropyl, 1-cyano-1-cyclopropyl, 1-piperidinyl, 1-pyrrolidinylmethyl, morpholin-4-yl, 4-methylpiperazin-1-yl, 4-methylpiperazin-1-ylmethyl, 4-tert-butyloxycarbonyl-1-piperazinyl, morpholin-4-ylmethyl, 1-pyrrolidinyl, phenyl, 4-cyanophenyl, 4-hydroxyphenyl, 1-pyrazolyl, 2-pyridinyl, 3-pyridinyl, 4-pyridinyl, hydroxy, methoxy, difluoromethoxy, trifluoromethoxy, benzyloxy, (3-methoxybenzyl)oxy, 3-pyridinyloxy, 3-(4-morpholinyl)propoxy, CONH₂, CONHMe, CONMe₂, CONH-cyclopentyl, CONH-cyclohexyl, CONH-benzyl, CO-(4-morpholinyl), CO-(4-methylpiperazin-1-yl), NH₂, NMe₂, NHCOPh, SO₂Me, SO₂NH-cyclohexyl and SO₂-(1-pyrrolidinyl).

In another embodiment, Ar is phenyl, optionally substituted with one or more substituents each independently selected from chloro, fluoro, cyano, methyl, isopropyl, isobutyl, tert-butyl, trifluoromethyl, difluoromethyl, hydroxymethyl, methoxymethyl, phenoxymethyl, dimethylaminomethyl, cyclopropyl, 1-cyano-1-cyclopropyl, 1-piperidinyl, 1-pyrrolidinylmethyl, morpholin-4-yl, 4-methylpiperazin-1-yl, 4-methylpiperazin-1-ylmethyl, 4-tert-butyloxycarbonyl-1-piperazinyl, morpholin-4-ylmethyl, 1-pyrrolidinyl, phenyl, 4-cyanophenyl, 4-hydroxyphenyl, 1-pyrazolyl, 2-pyridinyl, 3-pyridinyl, 4-pyridinyl, hydroxy, methoxy, benzyloxy, (3-methoxybenzyl)oxy, 3-pyridinyloxy, 3-(4-morpholinyl)propoxy, CONH₂, CONHMe, CONMe₂, CONH-cyclopentyl, CONH-cyclohexyl, CONH-benzyl, CO-(4-morpholinyl), CO-(4-methylpiperazin-1-yl), NH₂, NMe₂, NHCOPh, SO₂Me, SO₂NH-cyclohexyl and SO₂-(1-pyrrolidinyl).

In another embodiment, Ar is phenyl, optionally substituted with one or more substituents each independently selected from chloro, fluoro, cyano, methyl, isopropyl, isobutyl, tert-butyl, trifluoromethyl, difluoromethyl, hydroxymethyl, methoxymethyl, dimethylaminomethyl, cyclopropyl, 1-cyano-1-cyclopropyl, morpholin-4-yl, 4-methylpiperazin-1-yl, 4-tert-butyloxycarbonyl-1-piperazinyl, morpholin-4-ylmethyl, 1-pyrrolidinyl, phenyl, 1-pyrazolyl, 3-pyridinyl, 4-pyridinyl, hydroxy, methoxy, benzyloxy, 3-pyridinyloxy, 3-(4-morpholinyl)propoxy, CONH₂, CONHMe, CONMe₂, CONH-cyclopentyl, CONH-benzyl, CO-(4-morpholinyl), CO-(4-methylpiperazin-1-yl), NH₂, NMe₂, NHCOPh, SO₂Me and SO₂-(1-pyrrolidinyl).

In another embodiment, Ar is phenyl, optionally substituted with one or more substituents each independently selected from chloro, fluoro, cyano, methyl, isopropyl, isobutyl, trifluoromethyl, difluoromethyl, methoxymethyl, morpholin-4-yl, 4-methylpiperazin-1-yl, 4-tert-butyloxycarbonyl-1-piperazinyl, morpholin-4-ylmethyl, pyrrolidinyl, phenyl, 1-pyrazolyl, 4-pyridinyl, hydroxy, methoxy, benzyloxy, 3-pyridinyloxy, 3-(4-morpholinyl)propoxy, CONH₂, CONHMe, CONMe₂, CONH-cyclopentyl, CONH-benzyl, CO-(4-morpholinyl), CO-(4-methylpiperazin-1-yl), NH₂, NMe₂, NHCOPh, SO₂Me and SO₂-(1-pyrrolidinyl).

In another embodiment, Ar is phenyl, optionally substituted with one or more substituents each independently selected from chloro, fluoro, cyano, methyl, ethyl, isobutyl, tert-butyl, difluoromethyl, trifluoromethyl, hydroxymethyl, methoxymethyl, dimethylaminomethyl, cyclopropyl, 1-cyano-1-cyclopropyl, 1-pyrrolidinyl, 1-pyrazolyl, 3-pyridinyl, hydroxy, methoxy, CONH₂, CONHMe, CONMe₂, NH₂ and NMe₂.

In another embodiment, Ar is unsubstituted phenyl, unsubstituted 4-biphenyl or unsubstituted 1-naphthyl. In another embodiment, Ar is unsubstituted phenyl.

In another embodiment, Ar is thienyl, optionally substituted with one or more substituents each independently selected from halo, cyano, alkyl, alkenyl, alkynyl, haloalkyl, cycloalkyl, heterocyclyl, aryl, heteroaryl, cycloalkylalkyl, heterocyclylalkyl, COR⁸, OR⁹, NR¹⁰R¹¹ and S(O)ₙR¹².

In another embodiment, Ar is thienyl, optionally substituted with one or more substituents each independently selected from halo, cyano, alkyl, haloalkyl, cycloalkyl, heterocyclyl, heteroaryl, heterocyclylalkyl, COR⁸, OR⁹, NR¹⁰R¹¹ and S(O)ₙR¹².

In another embodiment, Ar is thienyl, optionally substituted with one or more substituents each independently selected from halo, cyano, alkyl, haloalkyl, cycloalkyl, heterocyclyl, heteroaryl, heterocyclylalkyl, CONR¹⁴R¹⁵, OR⁹, NR¹⁰R¹¹ and S(O)₂R¹².

In another embodiment, Ar is thienyl, optionally substituted with one or more substituents each independently selected from chloro, fluoro, cyano, methyl, isopropyl, isobutyl, tert-butyl, trifluoromethyl, difluoromethyl, hydroxymethyl, methoxymethyl, phenoxymethyl, dimethylaminomethyl, cyclopropyl, 1-cyano-1-cyclopropyl, 1-piperidinyl, 1-pyrrolidinylmethyl, morpholin-4-yl, 4-methylpiperazin-1-yl, 4-methylpiperazin-1-ylmethyl, 4-tert-butyloxycarbonyl-1-piperazinyl, morpholin-4-ylmethyl, 1-pyrrolidinyl, phenyl, 4-cyanophenyl, 4-hydroxyphenyl, 1-pyrazolyl, 2-pyridinyl, 3-pyridinyl, 4-pyridinyl, hydroxy, methoxy, benzyloxy, 3-methoxybenzyloxy, 3-pyridinyloxy, 3-(4-morpholinyl)propoxy, CONH₂, CONHMe, CONMe₂, CONH-cyclopentyl, CONH-cyclohexyl, CONH-benzyl, CO-(4-morpholinyl), CO-(4-methylpiperazin-1-yl), NH₂, NMe₂, NHCOPh, SO₂Me, SO₂NH-cyclohexyl and SO₂-(1-pyrrolidinyl).

In another embodiment, Ar is thienyl, optionally substituted with one or more substituents each independently selected from chloro, fluoro, cyano, methyl, isopropyl, isobutyl, trifluoromethyl, difluoromethyl, methoxymethyl, morpholin-4-yl, 4-methylpiperazin-1-yl, 4-tert-butyloxycarbonyl-1-piperazinyl, morpholin-4-ylmethyl, pyrrolidinyl, phenyl, 1-pyrazolyl, 4-pyridinyl, hydroxy, methoxy, benzyloxy, 3-pyridinyloxy, 3-(4-morpholinyl)propoxy, CONH₂, CONHMe, CONMe₂, CONH-cyclopentyl, CONH-benzyl, CO-(4-morpholinyl), CO-(4-methylpiperazin-1-yl), NH₂, NMe₂, NHCOPh, SO₂Me and SO₂-(1-pyrrolidinyl).

In another embodiment, Ar is thienyl, optionally substituted with one or more substituents each independently selected from chloro, fluoro, cyano, methyl, ethyl, isobutyl, tert-butyl, difluoromethyl, trifluoromethyl, hydroxymethyl, methoxymethyl, dimethylaminomethyl, cyclopropyl, 1-cyano-1-cyclopropyl, 1-pyrrolidinyl, 1-pyrazolyl, 3-pyridinyl, hydroxy, methoxy, CONH₂, CONHMe, CONMe₂, NH₂ and NMe₂.

In another embodiment, Ar is unsubstituted thienyl.

In another embodiment, Ar is pyrazolyl, optionally substituted with one or more substituents each independently selected from halo, cyano, alkyl, alkenyl, alkynyl, haloalkyl, cycloalkyl, heterocyclyl, aryl, heteroaryl, cycloalkylalkyl, heterocyclylalkyl, COR⁸, OR⁹, NR¹⁰R¹¹ and S(O)ₙR¹².

In another embodiment, Ar is pyrazolyl, optionally substituted with one or more substituents each independently selected from halo, cyano, alkyl, haloalkyl, cycloalkyl, heterocyclyl, heteroaryl, heterocyclylalkyl, COR⁸, OR⁹, NR¹⁰R¹¹ and S(O)ₙR¹².

In another embodiment, Ar is pyrazolyl, optionally substituted with one or more substituents each independently selected from halo, cyano, alkyl, haloalkyl, cycloalkyl, heterocyclyl, heteroaryl, heterocyclylalkyl, CONR¹⁴R¹⁵, OR⁹, NR¹⁰R¹¹ and S(O)₂R¹².

In another embodiment, Ar is pyrazolyl, optionally substituted with one or more substituents each independently selected from chloro, fluoro, cyano, methyl, isopropyl, isobutyl, tert-butyl, trifluoromethyl, difluoromethyl, hydroxymethyl, methoxymethyl, phenoxymethyl, dimethylaminomethyl, cyclopropyl, 1-cyano-1-cyclopropyl, 1-piperidinyl, 1-pyrrolidinylmethyl, morpholin-4-yl, 4-methylpiperazin-1-yl, 4-methylpiperazin-1-ylmethyl, 4-tert-butyloxycarbonyl-1-piperazinyl, morpholin-4-ylmethyl, 1-pyrrolidinyl, phenyl, 4-cyanophenyl, 4-hydroxyphenyl, 1-pyrazolyl, 2-pyridinyl, 3-pyridinyl, 4-pyridinyl, hydroxy, methoxy, benzyloxy, 3-methoxybenzyloxy, 3-pyridinyloxy, 3-(4-morpholinyl)propoxy, CONH₂, CONHMe, CONMe₂, CONH-cyclopentyl, CONH-cyclohexyl, CONH-benzyl, CO-(4-morpholinyl), CO-(4-methylpiperazin-1-yl), NH₂, NMe₂, NHCOPh, SO₂Me, SO₂NH-cyclohexyl and SO₂-(1-pyrrolidinyl).

In another embodiment, Ar is pyrazolyl, optionally substituted with one or more substituents each independently selected from chloro, fluoro, cyano, methyl, isopropyl, isobutyl, trifluoromethyl, difluoromethyl, methoxymethyl, morpholin-4-yl, 4-methylpiperazin-1-yl, 4-tert-butyloxycarbonyl-1-piperazinyl, morpholin-4-ylmethyl, pyrrolidinyl, phenyl, 1-pyrazolyl, 4-pyridinyl, hydroxy, methoxy, benzyloxy, 3-pyridinyloxy, 3-(4-morpholinyl)propoxy, CONH₂, CONHMe, CONMe₂, CONH-cyclopentyl, CONH-benzyl, CO-(4-morpholinyl), CO-(4-methylpiperazin-1-yl), NH₂, NMe₂, NHCOPh, SO₂Me and SO₂-(1-pyrrolidinyl).

In another embodiment, Ar is pyrazolyl, optionally substituted with one or more substituents each independently selected from chloro, fluoro, cyano, methyl, ethyl, isobutyl, tert-butyl, difluoromethyl, trifluoromethyl, hydroxymethyl, methoxymethyl, dimethylaminomethyl, cyclopropyl, 1-cyano-1-cyclopropyl, 1-pyrrolidinyl, 1-pyrazolyl, 3-pyridinyl, hydroxy, methoxy, CONH₂, CONHMe, CONMe₂, NH₂ and NMe₂.

In another embodiment, Ar is unsubstituted pyrazolyl.

In another embodiment, the compounds provided herein have the structure:

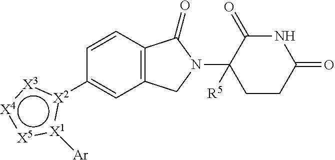

where R⁵, Ar and X¹-X⁵ are as defined elsewhere herein.

In another embodiment, the compounds provided herein have the structure:

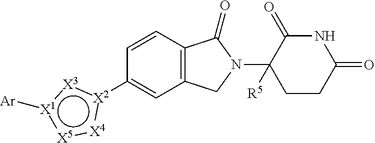

where R⁵, Ar and X¹-X⁵ are as defined elsewhere herein.

In another embodiment, the compounds provided herein have the structure:

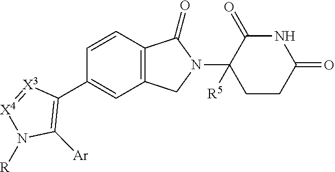

where X³, X⁴, R, Ar and R⁵ are as defined elsewhere herein.

In another embodiment, the compounds provided herein have the structure:

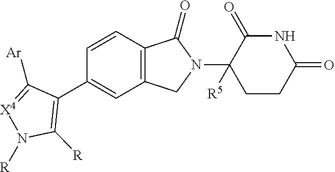

where X⁴, R, Ar and R⁵ are as defined elsewhere herein.

In another embodiment, the compounds provided herein have the structure:

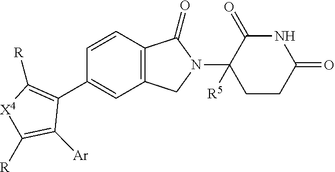

where X⁴, R, Ar and R⁵ are as defined elsewhere herein.

In another embodiment, the compounds provided herein have the structure:

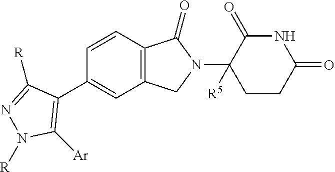

where R, R⁵ and Ar are as defined elsewhere herein.

In another embodiment, the compounds provided herein have the structure:

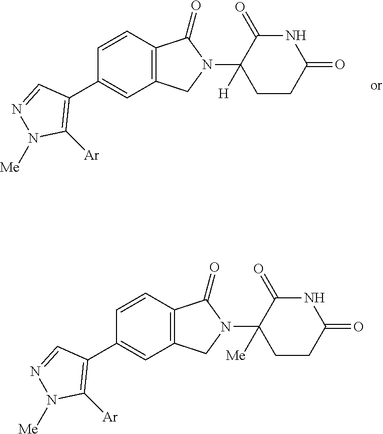

where Ar is as defined elsewhere herein.

In another embodiment, the compounds provided herein have the structure:

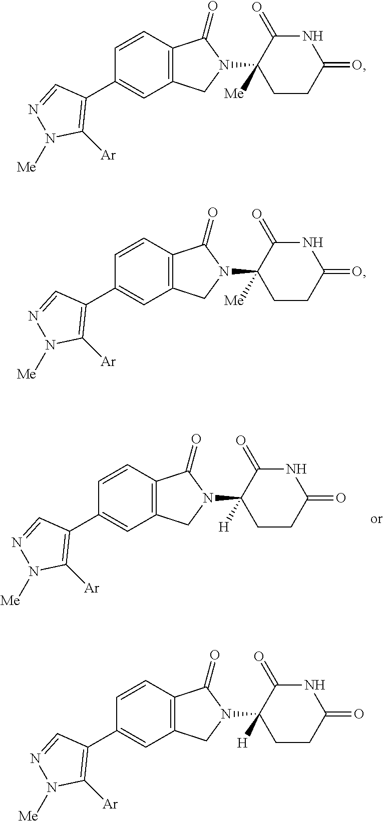

where Ar is as defined elsewhere herein. In another embodiment, Ar is phenyl, optionally substituted with one or more substituents each independently selected from halo, cyano, alkyl, haloalkyl, cycloalkyl, heterocyclyl, heteroaryl, heterocyclylalkyl, COR⁸, OR⁹, NR¹⁰R¹¹ and S(O)ₙR¹². In another embodiment, Ar is phenyl, optionally substituted with one or more substituents each independently selected from halo, cyano, alkyl, haloalkyl, cycloalkyl, heterocyclyl, heteroaryl, heterocyclylalkyl, CONR¹⁴R¹⁵, OR⁹, NR¹⁰R¹¹ and S(O)₂R¹². In another embodiment, Ar is phenyl, optionally substituted with one or more substituents each independently selected from chloro, fluoro, cyano, methyl, isopropyl, isobutyl, trifluoromethyl, difluoromethyl, methoxymethyl, morpholin-4-yl, 4-methylpiperazin-1-yl, 4-tert-butyloxycarbonyl-1-piperazinyl, morpholin-4-ylmethyl, pyrrolidinyl, phenyl, 1-pyrazolyl, 4-pyridinyl, hydroxy, methoxy, benzyloxy, 3-pyridinyloxy, 3-(4-morpholinyl)propoxy, CONH₂, CONHMe, CONMe₂, CONH-cyclopentyl, CONH-benzyl, CO-(4-morpholinyl), CO-(4-methylpiperazin-1-yl), NH₂, NMe₂, NHCOPh, SO₂Me and SO₂-(1-pyrrolidinyl).

In another embodiment, the compounds provided herein have the structure:

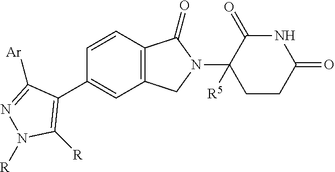

where R, R⁵ and Ar are as defined elsewhere herein.

In another embodiment, the compounds provided herein have the structure:

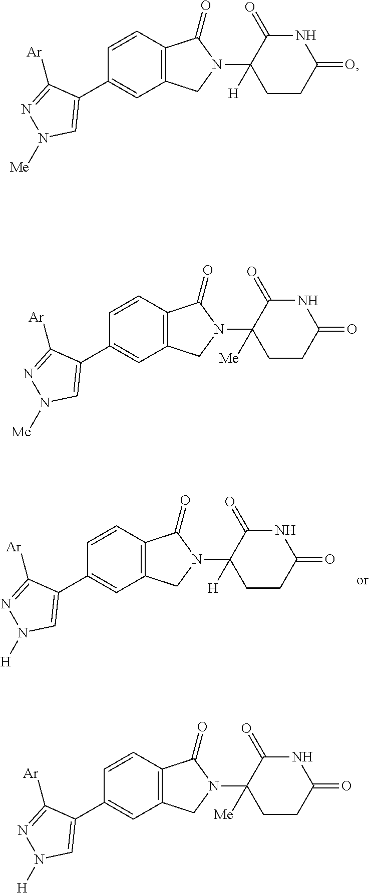

where Ar is as defined elsewhere herein.

In another embodiment, the compounds provided herein have the structure:

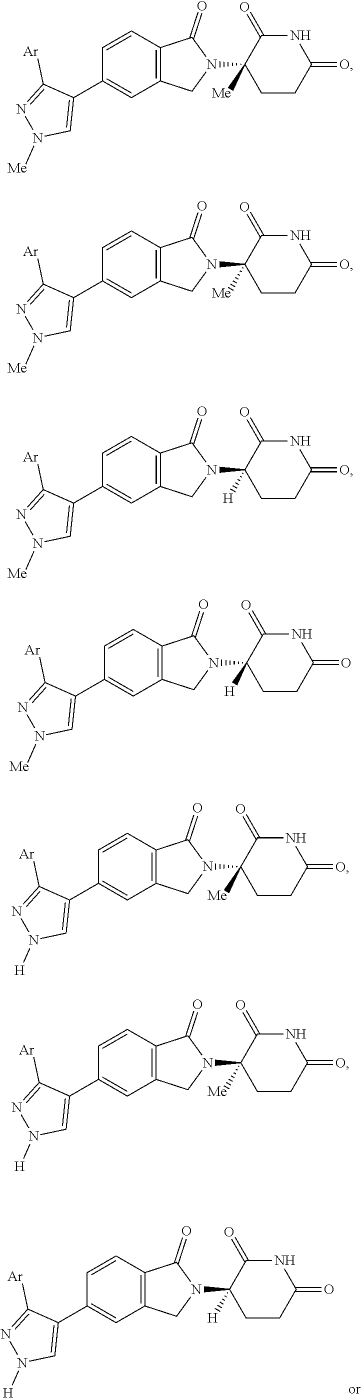

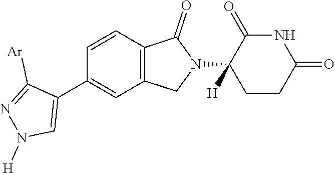

where Ar is as defined elsewhere herein. In another embodiment, Ar is phenyl, optionally substituted with one or more substituents each independently selected from halo, cyano, alkyl, haloalkyl, cycloalkyl, heterocyclyl, heteroaryl, heterocyclylalkyl, COR⁸, OR⁹, NR¹⁰R¹¹ and S(O)ₙR¹². In another embodiment, Ar is phenyl, optionally substituted with one or more substituents each independently selected from halo, cyano, alkyl, haloalkyl, cycloalkyl, heterocyclyl, heteroaryl, heterocyclylalkyl, CONR¹⁴R¹⁵, OR⁹, NR¹⁰R¹¹ and S(O)₂R¹². In another embodiment, Ar is phenyl, optionally substituted with one or more substituents each independently selected from chloro, fluoro, cyano, methyl, isopropyl, isobutyl, trifluoromethyl, difluoromethyl, methoxymethyl, morpholin-4-yl, 4-methylpiperazin-1-yl, 4-tert-butyloxycarbonyl-1-piperazinyl, morpholin-4-ylmethyl, pyrrolidinyl, phenyl, 1-pyrazolyl, 4-pyridinyl, hydroxy, methoxy, benzyloxy, 3-pyridinyloxy, 3-(4-morpholinyl)propoxy, CONH₂, CONHMe, CONMe₂, CONH-cyclopentyl, CONH-benzyl, CO-(4-morpholinyl), CO-(4-methylpiperazin-1-yl), NH₂, NMe₂, NHCOPh, SO₂Me and SO₂-(1-pyrrolidinyl).

In another embodiment, the compounds provided herein have the structure:

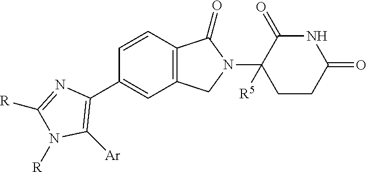

where R, R⁵ and Ar are as defined elsewhere herein.

In another embodiment, the compounds provided herein have the structure:

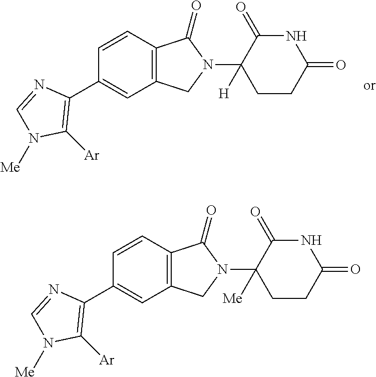

where Ar is as defined elsewhere herein.

In another embodiment, the compounds provided herein have the structure:

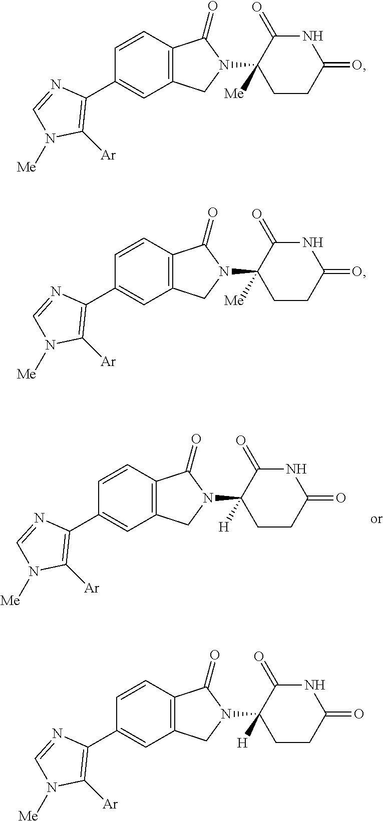

where Ar is as defined elsewhere herein. In another embodiment, Ar is phenyl, optionally substituted with one or more substituents each independently selected from halo, cyano, alkyl, haloalkyl, cycloalkyl, heterocyclyl, heteroaryl, heterocyclylalkyl, COR⁸, OR⁹, NR¹⁰R¹¹ and S(O)ₙR¹². In another embodiment, Ar is phenyl, optionally substituted with one or more substituents each independently selected from halo, cyano, alkyl, haloalkyl, cycloalkyl, heterocyclyl, heteroaryl, heterocyclylalkyl, CONR¹⁴R¹⁵, OR⁹, NR¹⁰R¹¹ and S(O)₂R¹². In another embodiment, Ar is phenyl, optionally substituted with one or more substituents each independently selected from chloro, fluoro, cyano, methyl, ethyl, isobutyl, tert-butyl, difluoromethyl, trifluoromethyl, hydroxymethyl, methoxymethyl, dimethylaminomethyl, cyclopropyl, 1-cyano-1-cyclopropyl, 1-pyrrolidinyl, 1-pyrazolyl, 3-pyridinyl, hydroxy, methoxy, CONH₂, CONHMe, CONMe₂, NH₂ and NMe₂.

In another embodiment, the compounds provided herein have the structure:

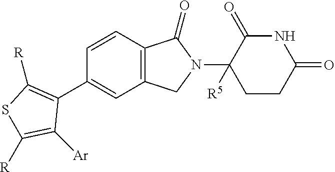

where R, Ar and R⁵ are as defined elsewhere herein.

In another embodiment, the compounds provided herein have the structure:

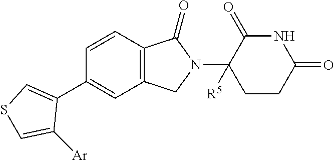

where Ar and R⁵ are as defined elsewhere herein.

In another embodiment, the compounds provided herein have the structure:

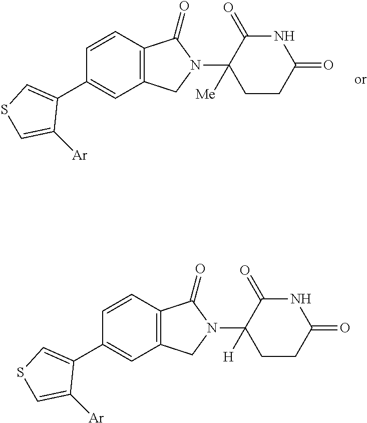

where Ar is as defined elsewhere herein.

In another embodiment, the compounds provided herein have the structure:

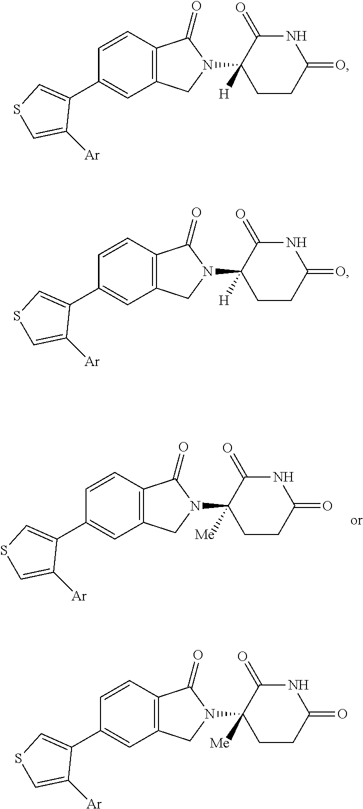

where Ar is as defined elsewhere herein. In another embodiment, Ar is phenyl, optionally substituted with one or more substituents each independently selected from halo, cyano, alkyl, alkenyl, alkynyl, haloalkyl, cycloalkyl, heterocyclyl, aryl, heteroaryl, cycloalkylalkyl, heterocyclylalkyl, COR⁸, OR⁹, NR¹⁰R¹¹ and S(O)ₙR¹². In another embodiment, Ar is phenyl, optionally substituted with one or more substituents each independently selected from chloro, fluoro, cyano, methyl, isopropyl, isobutyl, tert-butyl, trifluoromethyl, difluoromethyl, hydroxymethyl, methoxymethyl, dimethylaminomethyl, cyclopropyl, 1-cyano-1-cyclopropyl, morpholin-4-yl, 4-methylpiperazin-1-yl, 4-tert-butyloxycarbonyl-1-piperazinyl, morpholin-4-ylmethyl, 1-pyrrolidinyl, phenyl, 1-pyrazolyl, 3-pyridinyl, 4-pyridinyl, hydroxy, methoxy, benzyloxy, 3-pyridinyloxy, 3-(4-morpholinyl)propoxy, CONH₂, CONHMe, CONMe₂, CONH-cyclopentyl, CONH-benzyl, CO-(4-morpholinyl), CO-(4-methylpiperazin-1-yl), NH₂, NMe₂, NHCOPh, SO₂Me and SO₂-(1-pyrrolidinyl).

In another embodiment, the compounds provided herein have the structure:

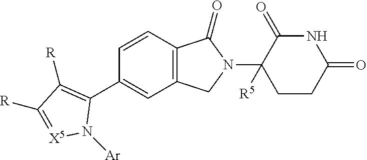

where X⁵, R, Ar and R⁵ are as defined elsewhere herein.

In another embodiment, the compounds provided herein have the structure:

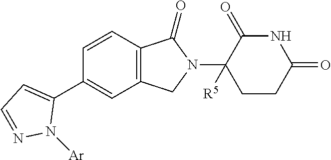

where Ar and R⁵ are as defined elsewhere herein.

In another embodiment, the compounds provided herein have the structure:

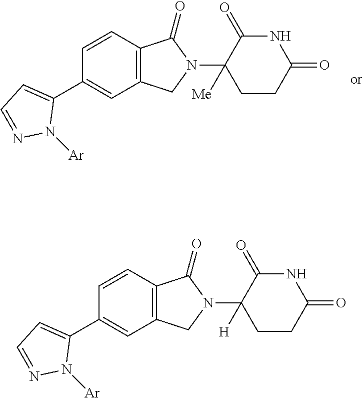

where Ar is as defined elsewhere herein.

In another embodiment, the compounds provided herein have the structure:

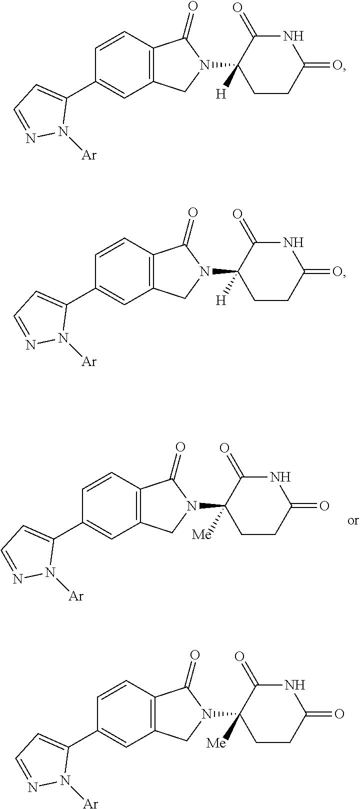

where Ar is as defined elsewhere herein. In another embodiment, Ar is phenyl, optionally substituted with one or more substituents each independently selected from halo, cyano, alkyl, haloalkyl, cycloalkyl, heterocyclyl, heteroaryl, heterocyclylalkyl, COR⁸, OR⁹, NR¹⁰R¹¹ and S(O)ₙR¹². In another embodiment, Ar is phenyl, optionally substituted with one or more substituents each independently selected from halo, cyano, alkyl, haloalkyl, cycloalkyl, heterocyclyl, heteroaryl, heterocyclylalkyl, CONR¹⁴R¹⁵, OR⁹, NR¹⁰R¹¹ and S(O)₂R¹². In another embodiment, Ar is phenyl, optionally substituted with one or more substituents each independently selected from chloro, fluoro, cyano, methyl, isopropyl, isobutyl, trifluoromethyl, difluoromethyl, methoxymethyl, morpholin-4-yl, 4-methylpiperazin-1-yl, 4-tert-butyloxycarbonyl-1-piperazinyl, morpholin-4-ylmethyl, pyrrolidinyl, phenyl, 1-pyrazolyl, 4-pyridinyl, hydroxy, methoxy, benzyloxy, 3-pyridinyloxy, 3-(4-morpholinyl)propoxy, CONH₂, CONHMe, CONMe₂, CONH-cyclopentyl, CONH-benzyl, CO-(4-morpholinyl), CO-(4-methylpiperazin-1-yl), NH₂, NMe₂, NHCOPh, SO₂Me and SO₂-(1-pyrrolidinyl).

In another embodiment, the compound provided herein for use in the compositions and methods provided herein is selected from:

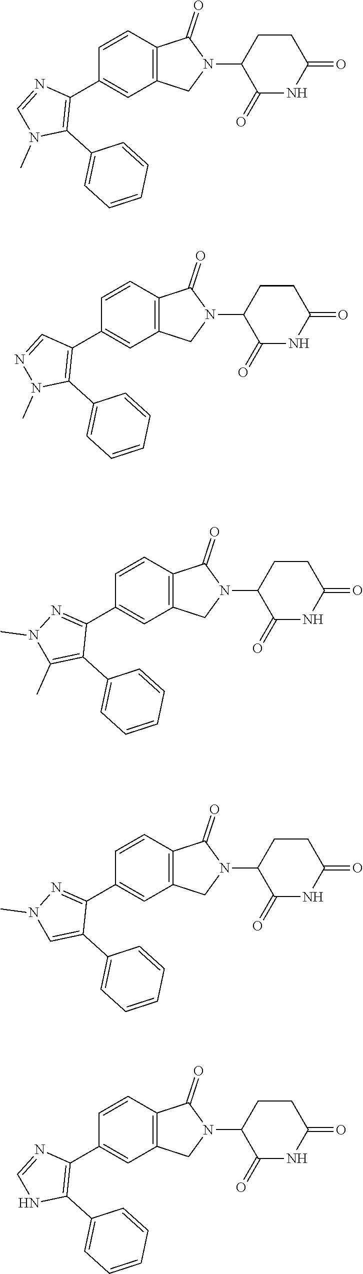 [SMILES: Cn1cnc(-c2ccc3c(c2)CN(C2CCC(=O)NC2=O)C3=O)c1-c1ccccc1 ; Cn1ncc(-c2ccc3c(c2)CN(C2CCC(=O)NC2=O)C3=O)c1-c1ccccc1 ; Cc1c(-c2ccccc2)c(-c2ccc3c(c2)CN(C2CCC(=O)NC2=O)C3=O)nn1C ; Cn1cc(-c2ccccc2)c(-c2ccc3c(c2)CN(C2CCC(=O)NC2=O)C3=O)n1 ; O=C1CCC(N2Cc3cc(-c4nc[nH]c4-c4ccccc4)ccc3C2=O)C(=O)N1]

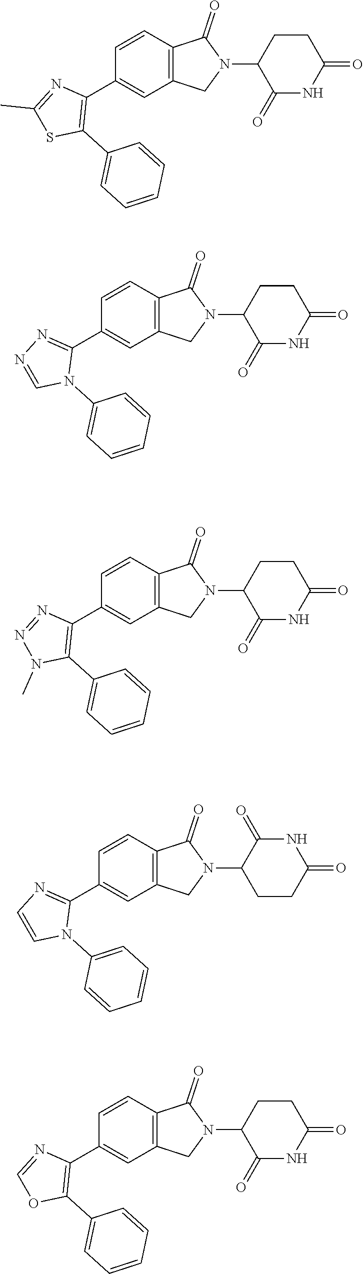 [SMILES: Cc1nc(-c2ccc3c(c2)CN(C2CCC(=O)NC2=O)C3=O)c(-c2ccccc2)s1 ; O=C1CCC(N2Cc3cc(-c4nncn4-c4ccccc4)ccc3C2=O)C(=O)N1 ; Cn1nnc(-c2ccc3c(c2)CN(C2CCC(=O)NC2=O)C3=O)c1-c1ccccc1 ; O=C1CCC(N2Cc3cc(-c4nccn4-c4ccccc4)ccc3C2=O)C(=O)N1 ; O=C1CCC(N2Cc3cc(-c4ncoc4-c4ccccc4)ccc3C2=O)C(=O)N1]

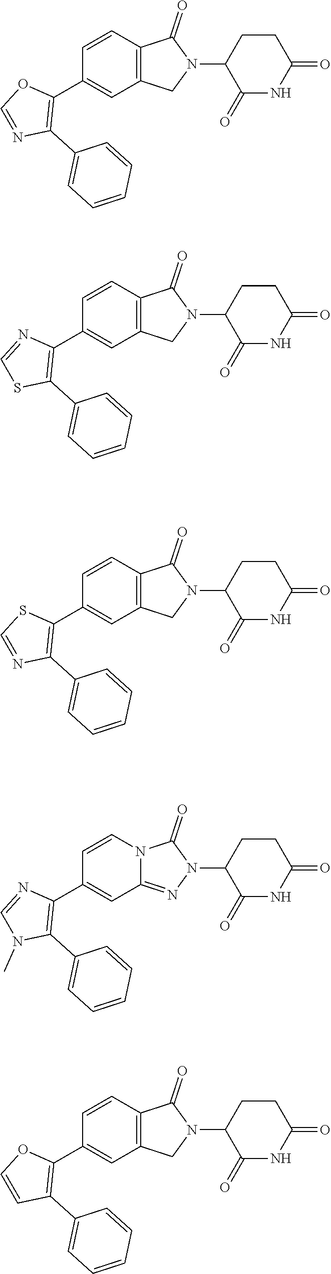 [SMILES: O=C1CCC(N2Cc3cc(-c4ocnc4-c4ccccc4)ccc3C2=O)C(=O)N1 ; O=C1CCC(N2Cc3cc(-c4ncsc4-c4ccccc4)ccc3C2=O)C(=O)N1 ; O=C1CCC(N2Cc3cc(-c4scnc4-c4ccccc4)ccc3C2=O)C(=O)N1 ; Cn1cnc(-c2ccn3c(=O)n(C4CCC(=O)NC4=O)nc3c2)c1-c1ccccc1 ; O=C1CCC(N2Cc3cc(-c4occc4-c4ccccc4)ccc3C2=O)C(=O)N1]

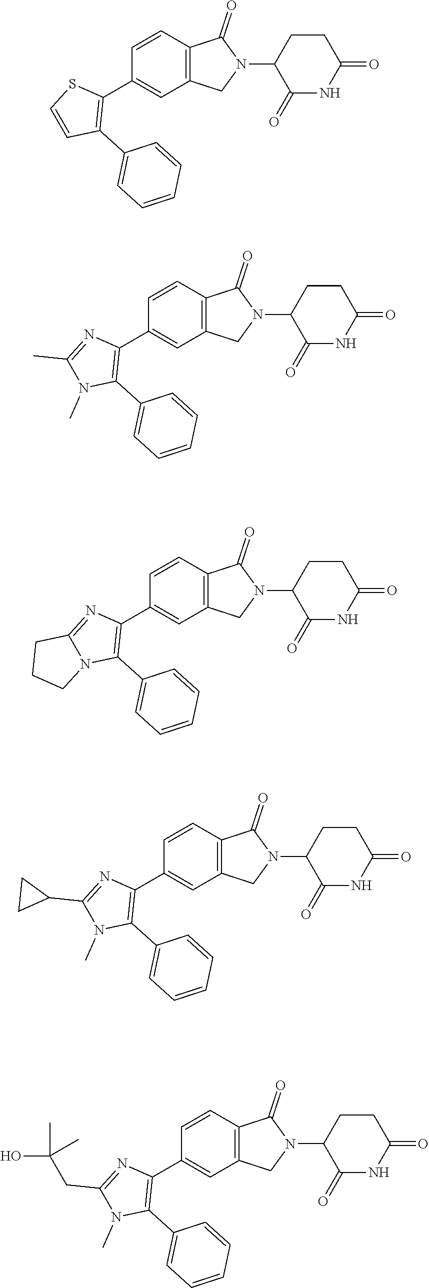 [SMILES: O=C1CCC(N2Cc3cc(-c4sccc4-c4ccccc4)ccc3C2=O)C(=O)N1 ; Cc1nc(-c2ccc3c(c2)CN(C2CCC(=O)NC2=O)C3=O)c(-c2ccccc2)n1C ; O=C1CCC(N2Cc3cc(-c4nc5n(c4-c4ccccc4)CCC5)ccc3C2=O)C(=O)N1 ; Cn1c(C2CC2)nc(-c2ccc3c(c2)CN(C2CCC(=O)NC2=O)C3=O)c1-c1ccccc1 ; Cn1c(CC(C)(C)O)nc(-c2ccc3c(c2)CN(C2CCC(=O)NC2=O)C3=O)c1-c1ccccc1]

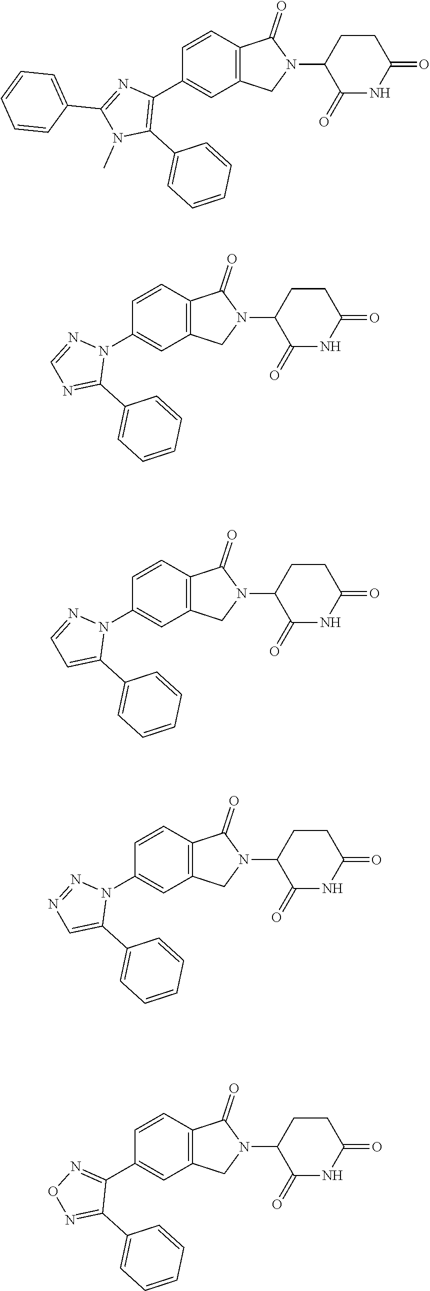 [SMILES: Cn1c(-c2ccccc2)nc(-c2ccc3c(c2)CN(C2CCC(=O)NC2=O)C3=O)c1-c1ccccc1 ; O=C1CCC(N2Cc3cc(-n4ncnc4-c4ccccc4)ccc3C2=O)C(=O)N1 ; O=C1CCC(N2Cc3cc(-n4nccc4-c4ccccc4)ccc3C2=O)C(=O)N1 ; O=C1CCC(N2Cc3cc(-n4nncc4-c4ccccc4)ccc3C2=O)C(=O)N1 ; O=C1CCC(N2Cc3cc(-c4nonc4-c4ccccc4)ccc3C2=O)C(=O)N1]

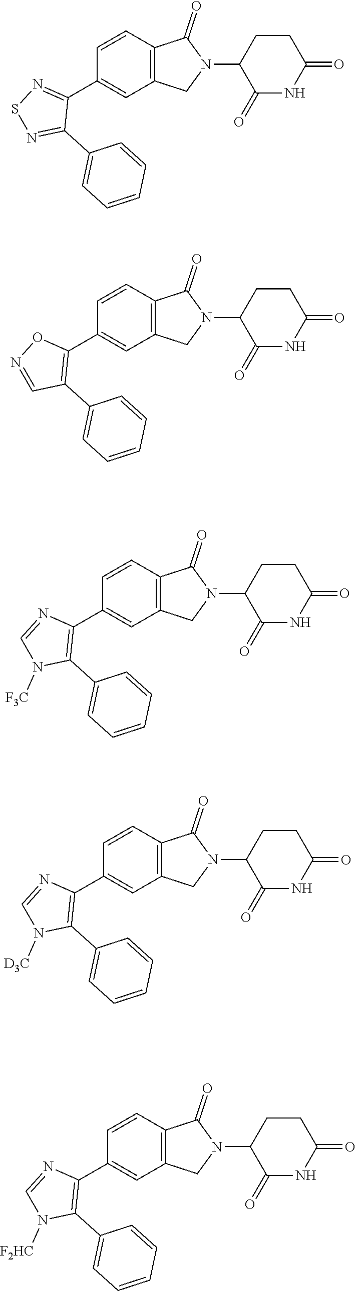 [SMILES: O=C1CCC(N2Cc3cc(-c4nsnc4-c4ccccc4)ccc3C2=O)C(=O)N1 ; O=C1CCC(N2Cc3cc(-c4oncc4-c4ccccc4)ccc3C2=O)C(=O)N1 ; O=C1CCC(N2Cc3cc(-c4ncn(C(F)(F)F)c4-c4ccccc4)ccc3C2=O)C(=O)N1 ; [2H]C([2H])([2H])n1cnc(-c2ccc3c(c2)CN(C2CCC(=O)NC2=O)C3=O)c1-c1ccccc1 ; O=C1CCC(N2Cc3cc(-c4ncn(C(F)F)c4-c4ccccc4)ccc3C2=O)C(=O)N1]

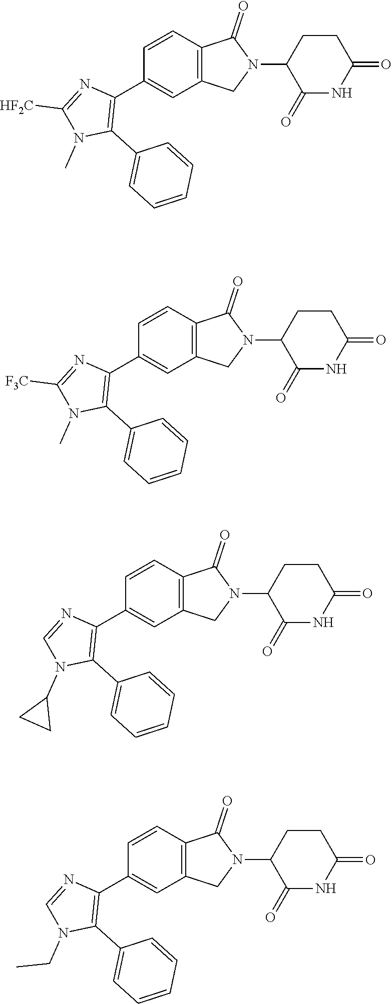

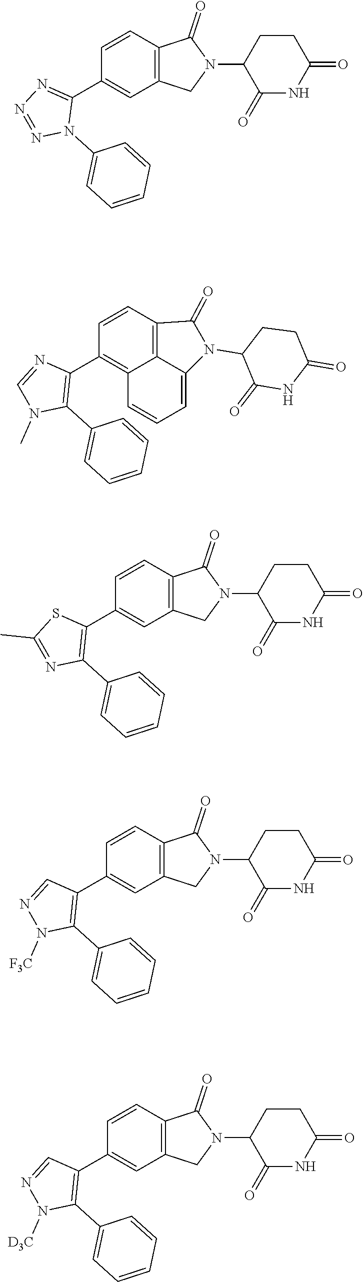 [SMILES: O=C1CCC(N2Cc3cc(-c4nnnn4-c4ccccc4)ccc3C2=O)C(=O)N1 ; Cn1cnc(-c2ccc3c4c(cccc24)N(C2CCC(=O)NC2=O)C3=O)c1-c1ccccc1 ; Cc1nc(-c2ccccc2)c(-c2ccc3c(c2)CN(C2CCC(=O)NC2=O)C3=O)s1 ; O=C1CCC(N2Cc3cc(-c4cnn(C(F)(F)F)c4-c4ccccc4)ccc3C2=O)C(=O)N1 ; [2H]C([2H])([2H])n1ncc(-c2ccc3c(c2)CN(C2CCC(=O)NC2=O)C3=O)c1-c1ccccc1]

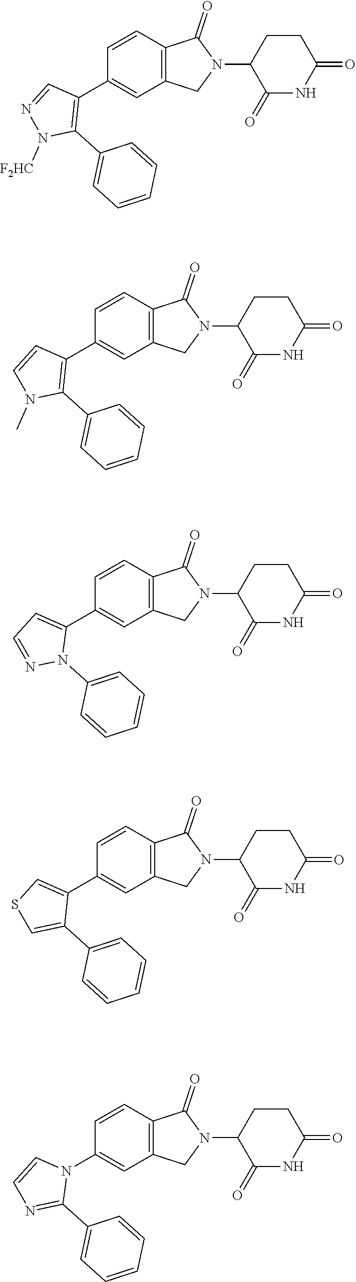 [SMILES: O=C1CCC(N2Cc3cc(-c4cnn(C(F)F)c4-c4ccccc4)ccc3C2=O)C(=O)N1 ; Cn1ccc(-c2ccc3c(c2)CN(C2CCC(=O)NC2=O)C3=O)c1-c1ccccc1 ; O=C1CCC(N2Cc3cc(-c4ccnn4-c4ccccc4)ccc3C2=O)C(=O)N1 ; O=C1CCC(N2Cc3cc(-c4cscc4-c4ccccc4)ccc3C2=O)C(=O)N1 ; O=C1CCC(N2Cc3cc(-n4ccnc4-c4ccccc4)ccc3C2=O)C(=O)N1]

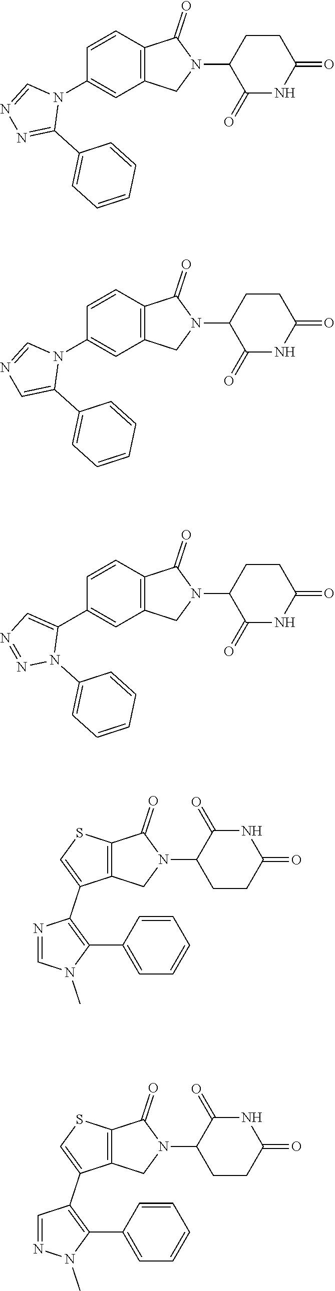 [SMILES: O=C1CCC(N2Cc3cc(-n4cnnc4-c4ccccc4)ccc3C2=O)C(=O)N1 ; O=C1CCC(N2Cc3cc(-n4cncc4-c4ccccc4)ccc3C2=O)C(=O)N1 ; O=C1CCC(N2Cc3cc(-c4cnnn4-c4ccccc4)ccc3C2=O)C(=O)N1 ; Cn1cnc(-c2csc3c2CN(C2CCC(=O)NC2=O)C3=O)c1-c1ccccc1 ; Cn1ncc(-c2csc3c2CN(C2CCC(=O)NC2=O)C3=O)c1-c1ccccc1]

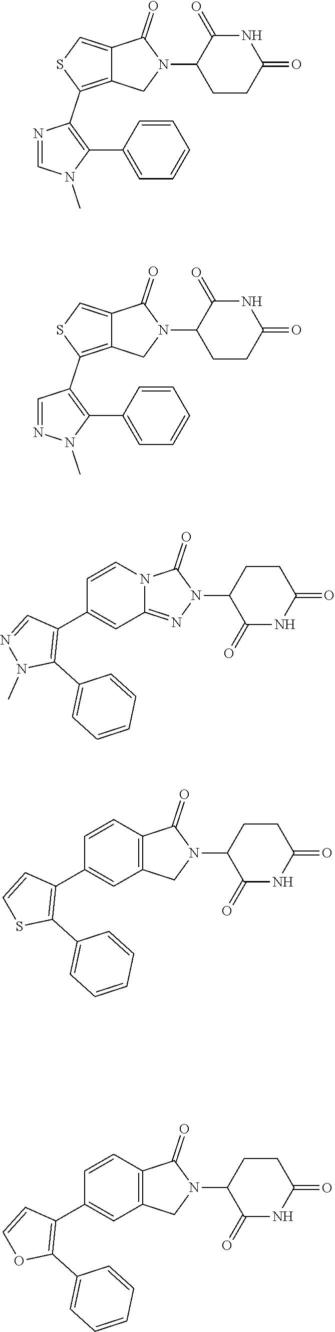 [SMILES: Cn1cnc(-c2scc3c2CN(C2CCC(=O)NC2=O)C3=O)c1-c1ccccc1 ; Cn1ncc(-c2scc3c2CN(C2CCC(=O)NC2=O)C3=O)c1-c1ccccc1 ; Cn1ncc(-c2ccn3c(=O)n(C4CCC(=O)NC4=O)nc3c2)c1-c1ccccc1 ; O=C1CCC(N2Cc3cc(-c4ccsc4-c4ccccc4)ccc3C2=O)C(=O)N1 ; O=C1CCC(N2Cc3cc(-c4ccoc4-c4ccccc4)ccc3C2=O)C(=O)N1]

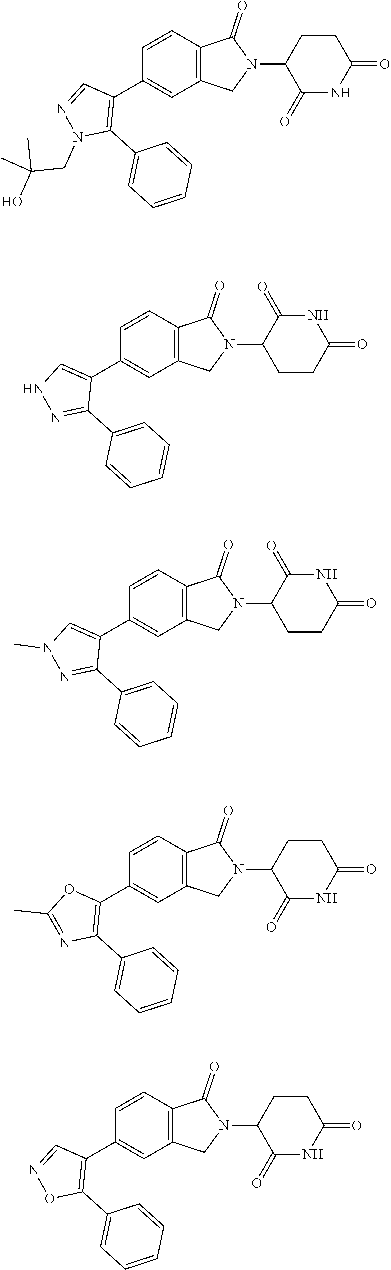 [SMILES: CC(C)(O)Cn1ncc(-c2ccc3c(c2)CN(C2CCC(=O)NC2=O)C3=O)c1-c1ccccc1 ; O=C1CCC(N2Cc3cc(-c4c[nH]nc4-c4ccccc4)ccc3C2=O)C(=O)N1 ; Cn1cc(-c2ccc3c(c2)CN(C2CCC(=O)NC2=O)C3=O)c(-c2ccccc2)n1 ; Cc1nc(-c2ccccc2)c(-c2ccc3c(c2)CN(C2CCC(=O)NC2=O)C3=O)o1 ; O=C1CCC(N2Cc3cc(-c4cnoc4-c4ccccc4)ccc3C2=O)C(=O)N1]

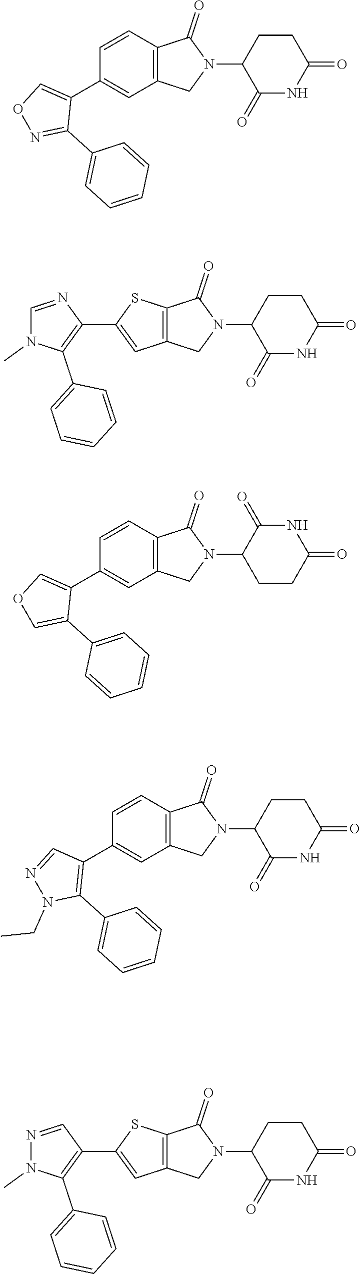 [SMILES: O=C1CCC(N2Cc3cc(-c4conc4-c4ccccc4)ccc3C2=O)C(=O)N1 ; Cn1cnc(-c2cc3c(s2)C(=O)N(C2CCC(=O)NC2=O)C3)c1-c1ccccc1 ; O=C1CCC(N2Cc3cc(-c4cocc4-c4ccccc4)ccc3C2=O)C(=O)N1 ; CCn1ncc(-c2ccc3c(c2)CN(C2CCC(=O)NC2=O)C3=O)c1-c1ccccc1 ; Cn1ncc(-c2cc3c(s2)C(=O)N(C2CCC(=O)NC2=O)C3)c1-c1ccccc1]

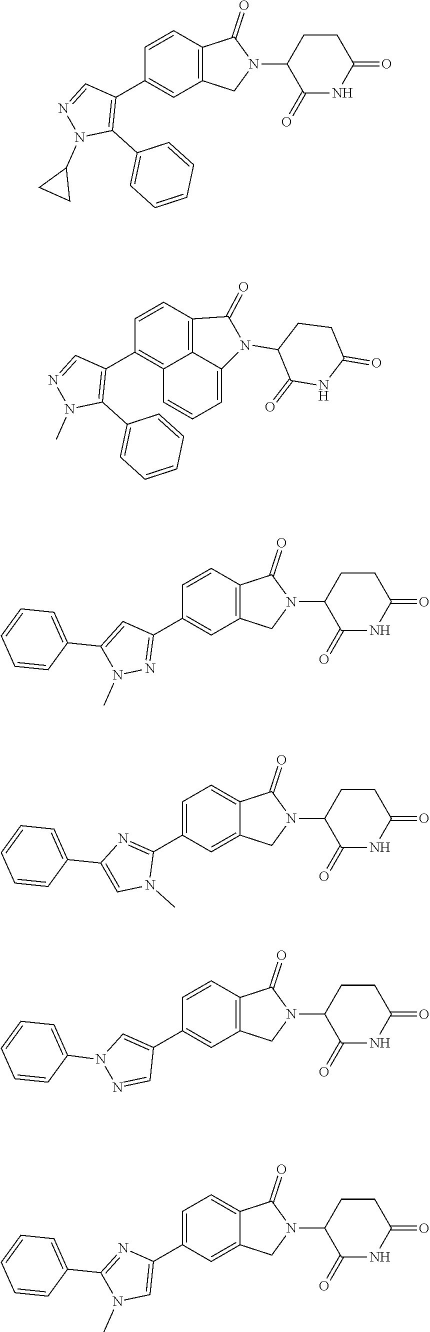 [SMILES: O=C1CCC(N2Cc3cc(-c4cnn(C5CC5)c4-c4ccccc4)ccc3C2=O)C(=O)N1 ; Cn1ncc(-c2ccc3c4c(cccc24)N(C2CCC(=O)NC2=O)C3=O)c1-c1ccccc1 ; Cn1nc(-c2ccc3c(c2)CN(C2CCC(=O)NC2=O)C3=O)cc1-c1ccccc1 ; Cn1cc(-c2ccccc2)nc1-c1ccc2c(c1)CN(C1CCC(=O)NC1=O)C2=O ; O=C1CCC(N2Cc3cc(-c4cnn(-c5ccccc5)c4)ccc3C2=O)C(=O)N1 ; Cn1cc(-c2ccc3c(c2)CN(C2CCC(=O)NC2=O)C3=O)nc1-c1ccccc1]

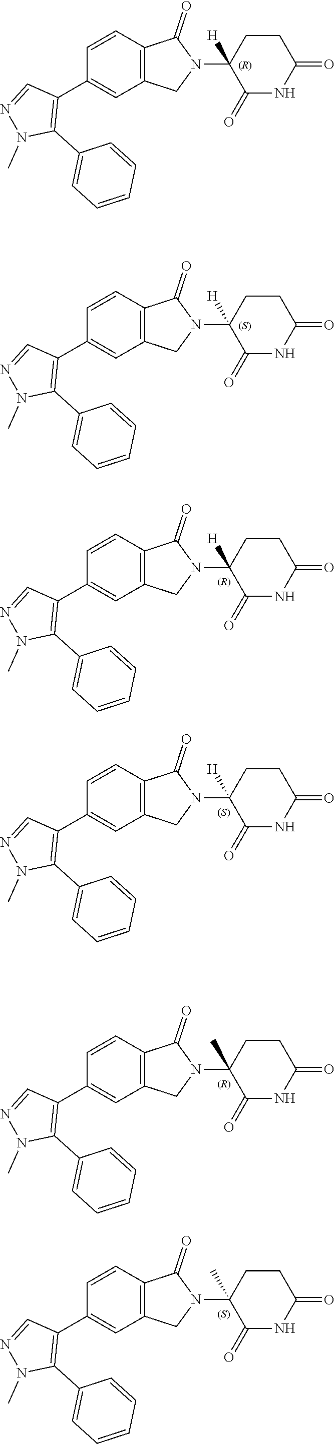

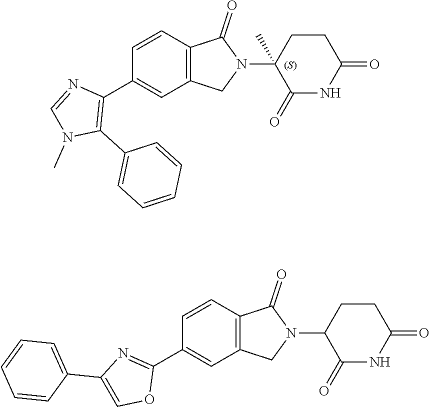

In another embodiment, the compound provided herein for use in the compositions and methods provided herein is selected from:

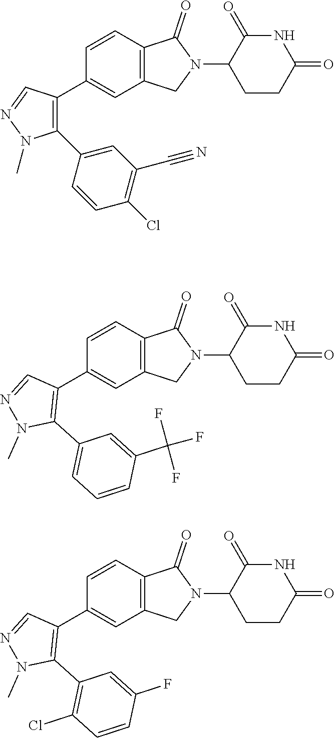 [SMILES: Cn1ncc(-c2ccc3c(c2)CN(C2CCC(=O)NC2=O)C3=O)c1-c1ccc(Cl)c(C#N)c1 ; Cn1ncc(-c2ccc3c(c2)CN(C2CCC(=O)NC2=O)C3=O)c1-c1cccc(C(F)(F)F)c1 ; Cn1ncc(-c2ccc3c(c2)CN(C2CCC(=O)NC2=O)C3=O)c1-c1cc(F)ccc1Cl]

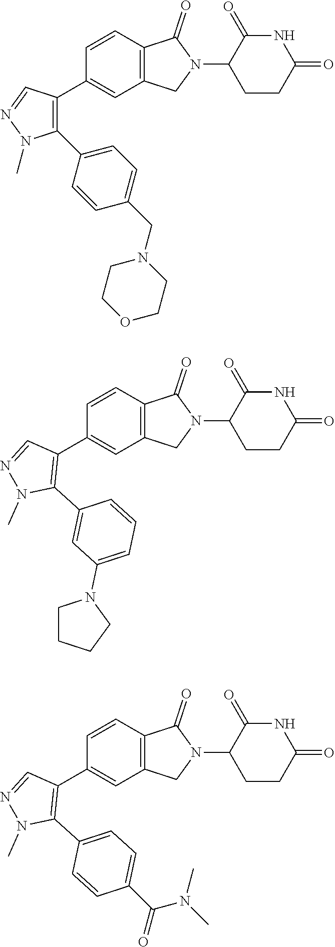 [SMILES: Cn1ncc(-c2ccc3c(c2)CN(C2CCC(=O)NC2=O)C3=O)c1-c1ccc(CN2CCOCC2)cc1 ; Cn1ncc(-c2ccc3c(c2)CN(C2CCC(=O)NC2=O)C3=O)c1-c1cccc(N2CCCC2)c1 ; CN(C)C(=O)c1ccc(-c2c(-c3ccc4c(c3)CN(C3CCC(=O)NC3=O)C4=O)cnn2C)cc1]

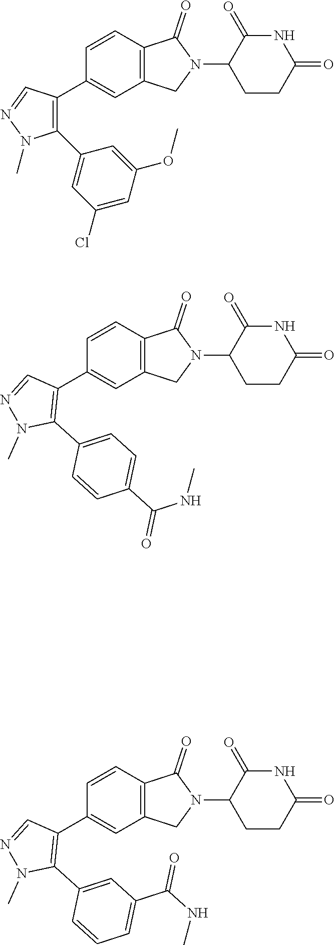 [SMILES: COc1cc(Cl)cc(-c2c(-c3ccc4c(c3)CN(C3CCC(=O)NC3=O)C4=O)cnn2C)c1 ; CNC(=O)c1ccc(-c2c(-c3ccc4c(c3)CN(C3CCC(=O)NC3=O)C4=O)cnn2C)cc1 ; CNC(=O)c1cccc(-c2c(-c3ccc4c(c3)CN(C3CCC(=O)NC3=O)C4=O)cnn2C)c1]

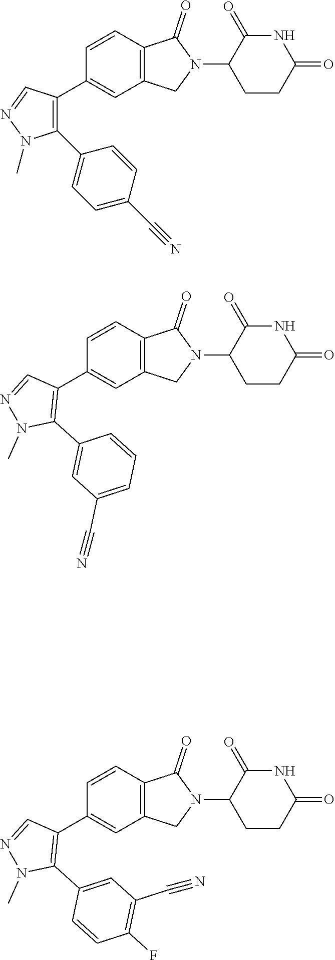 [SMILES: Cn1ncc(-c2ccc3c(c2)CN(C2CCC(=O)NC2=O)C3=O)c1-c1ccc(C#N)cc1 ; Cn1ncc(-c2ccc3c(c2)CN(C2CCC(=O)NC2=O)C3=O)c1-c1cccc(C#N)c1 ; Cn1ncc(-c2ccc3c(c2)CN(C2CCC(=O)NC2=O)C3=O)c1-c1ccc(F)c(C#N)c1]

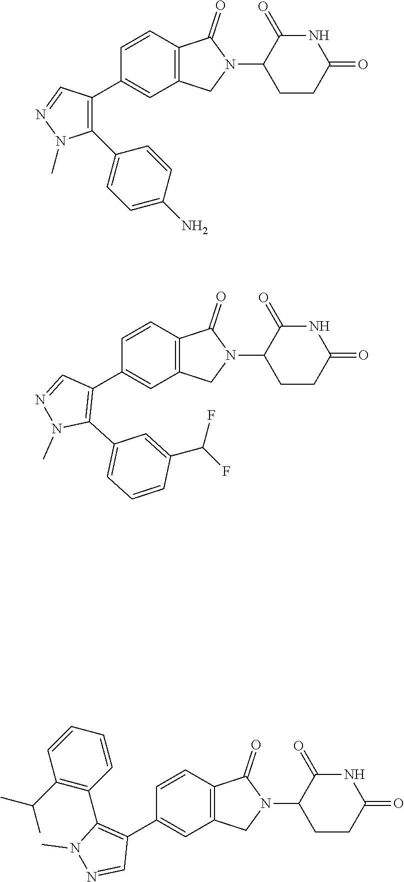 [SMILES: Cn1ncc(-c2ccc3c(c2)CN(C2CCC(=O)NC2=O)C3=O)c1-c1ccc(N)cc1 ; Cn1ncc(-c2ccc3c(c2)CN(C2CCC(=O)NC2=O)C3=O)c1-c1cccc(C(F)F)c1 ; CC(C)c1ccccc1-c1c(-c2ccc3c(c2)CN(C2CCC(=O)NC2=O)C3=O)cnn1C]

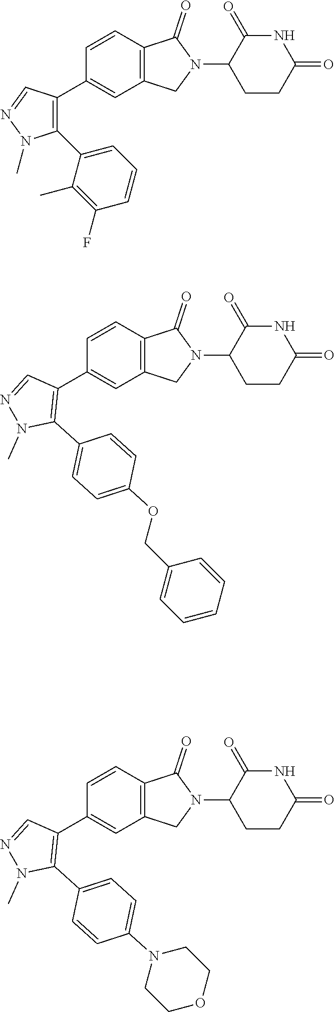 [SMILES: Cc1c(F)cccc1-c1c(-c2ccc3c(c2)CN(C2CCC(=O)NC2=O)C3=O)cnn1C ; Cn1ncc(-c2ccc3c(c2)CN(C2CCC(=O)NC2=O)C3=O)c1-c1ccc(OCc2ccccc2)cc1 ; Cn1ncc(-c2ccc3c(c2)CN(C2CCC(=O)NC2=O)C3=O)c1-c1ccc(N2CCOCC2)cc1]

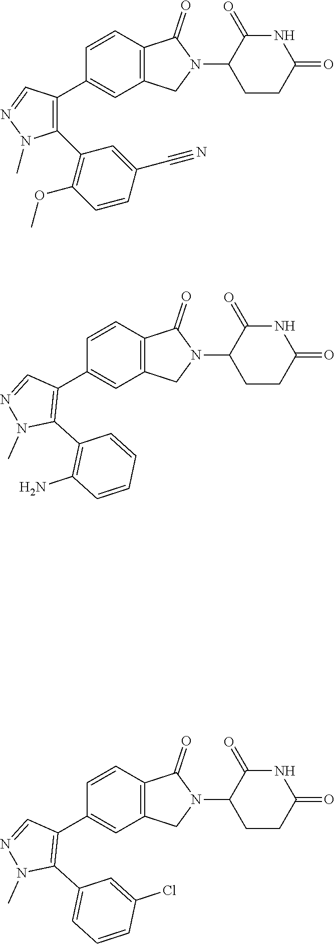 [SMILES: COc1ccc(C#N)cc1-c1c(-c2ccc3c(c2)CN(C2CCC(=O)NC2=O)C3=O)cnn1C ; Cn1ncc(-c2ccc3c(c2)CN(C2CCC(=O)NC2=O)C3=O)c1-c1ccccc1N ; Cn1ncc(-c2ccc3c(c2)CN(C2CCC(=O)NC2=O)C3=O)c1-c1cccc(Cl)c1]

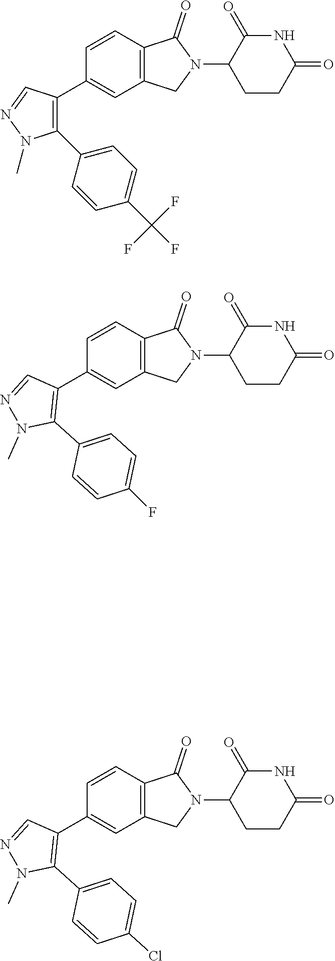 [SMILES: Cn1ncc(-c2ccc3c(c2)CN(C2CCC(=O)NC2=O)C3=O)c1-c1ccc(C(F)(F)F)cc1 ; Cn1ncc(-c2ccc3c(c2)CN(C2CCC(=O)NC2=O)C3=O)c1-c1ccc(F)cc1 ; Cn1ncc(-c2ccc3c(c2)CN(C2CCC(=O)NC2=O)C3=O)c1-c1ccc(Cl)cc1]

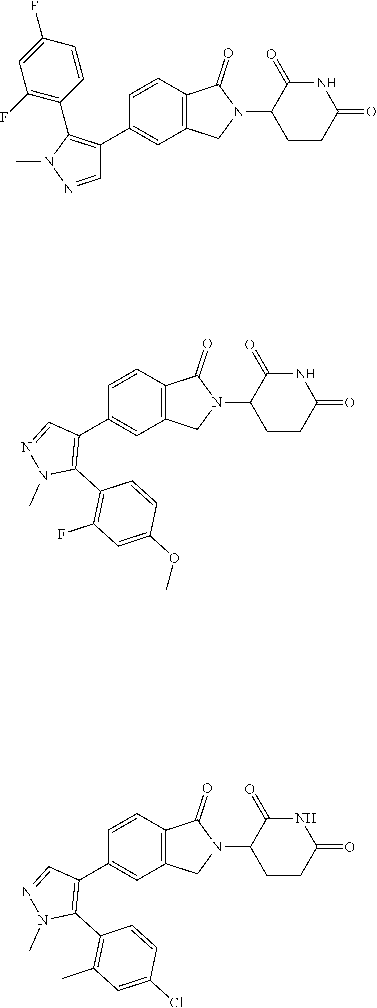 [SMILES: Cn1ncc(-c2ccc3c(c2)CN(C2CCC(=O)NC2=O)C3=O)c1-c1ccc(F)cc1F ; COc1ccc(-c2c(-c3ccc4c(c3)CN(C3CCC(=O)NC3=O)C4=O)cnn2C)c(F)c1 ; Cc1cc(Cl)ccc1-c1c(-c2ccc3c(c2)CN(C2CCC(=O)NC2=O)C3=O)cnn1C]

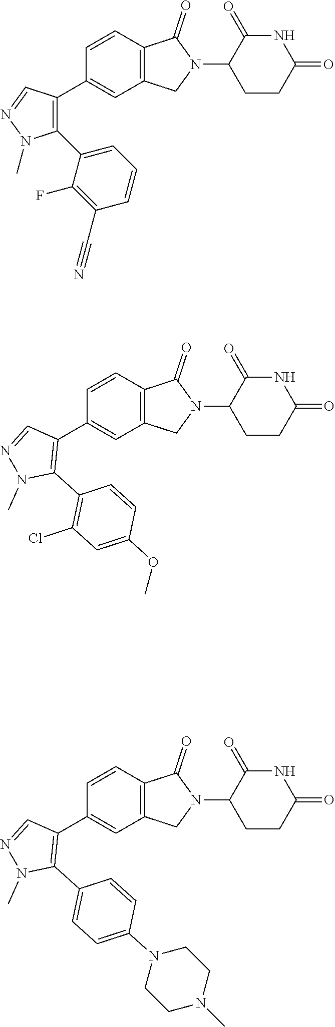 [SMILES: Cn1ncc(-c2ccc3c(c2)CN(C2CCC(=O)NC2=O)C3=O)c1-c1cccc(C#N)c1F ; COc1ccc(-c2c(-c3ccc4c(c3)CN(C3CCC(=O)NC3=O)C4=O)cnn2C)c(Cl)c1 ; CN1CCN(c2ccc(-c3c(-c4ccc5c(c4)CN(C4CCC(=O)NC4=O)C5=O)cnn3C)cc2)CC1]

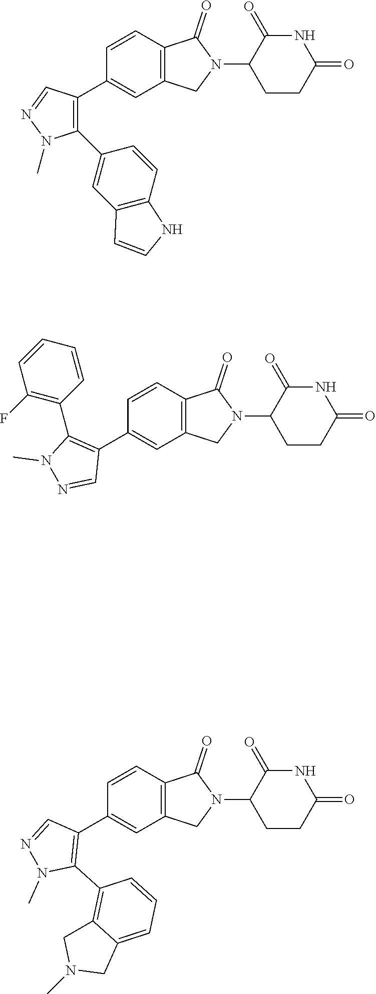 [SMILES: Cn1ncc(-c2ccc3c(c2)CN(C2CCC(=O)NC2=O)C3=O)c1-c1ccc2[nH]ccc2c1 ; Cn1ncc(-c2ccc3c(c2)CN(C2CCC(=O)NC2=O)C3=O)c1-c1ccccc1F ; CN1Cc2cccc(-c3c(-c4ccc5c(c4)CN(C4CCC(=O)NC4=O)C5=O)cnn3C)c2C1]

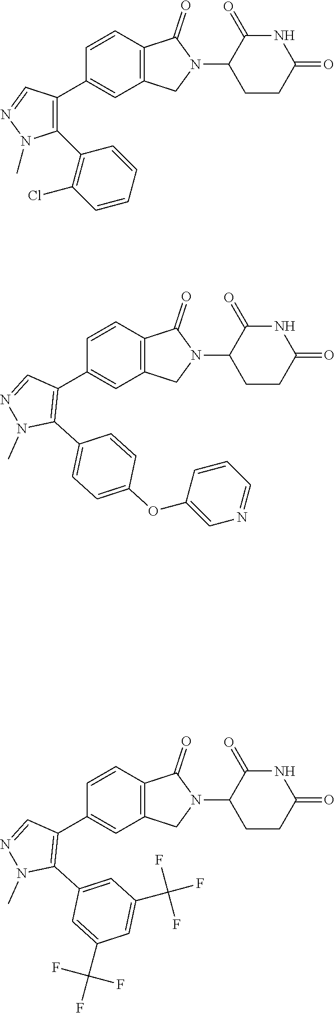 [SMILES: Cn1ncc(-c2ccc3c(c2)CN(C2CCC(=O)NC2=O)C3=O)c1-c1ccccc1Cl ; Cn1ncc(-c2ccc3c(c2)CN(C2CCC(=O)NC2=O)C3=O)c1-c1ccc(Oc2cccnc2)cc1 ; Cn1ncc(-c2ccc3c(c2)CN(C2CCC(=O)NC2=O)C3=O)c1-c1cc(C(F)(F)F)cc(C(F)(F)F)c1]

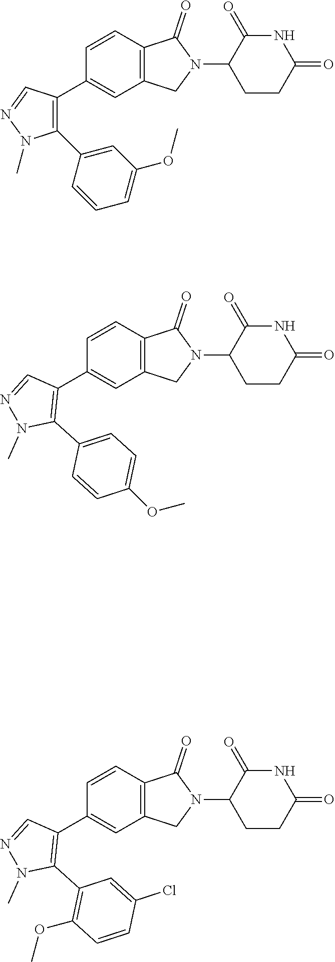 [SMILES: COc1cccc(-c2c(-c3ccc4c(c3)CN(C3CCC(=O)NC3=O)C4=O)cnn2C)c1 ; COc1ccc(-c2c(-c3ccc4c(c3)CN(C3CCC(=O)NC3=O)C4=O)cnn2C)cc1 ; COc1ccc(Cl)cc1-c1c(-c2ccc3c(c2)CN(C2CCC(=O)NC2=O)C3=O)cnn1C]

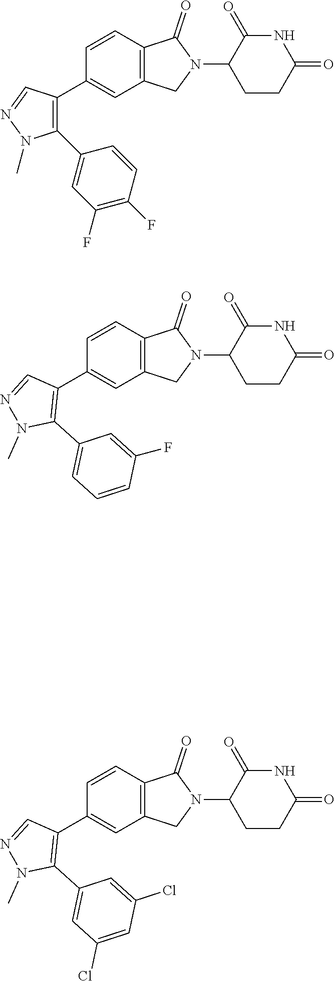 [SMILES: Cn1ncc(-c2ccc3c(c2)CN(C2CCC(=O)NC2=O)C3=O)c1-c1ccc(F)c(F)c1 ; Cn1ncc(-c2ccc3c(c2)CN(C2CCC(=O)NC2=O)C3=O)c1-c1cccc(F)c1 ; Cn1ncc(-c2ccc3c(c2)CN(C2CCC(=O)NC2=O)C3=O)c1-c1cc(Cl)cc(Cl)c1]

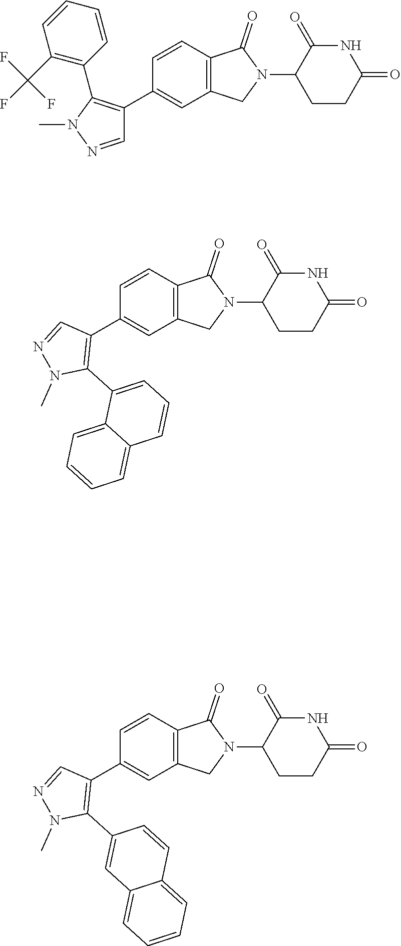 [SMILES: Cn1ncc(-c2ccc3c(c2)CN(C2CCC(=O)NC2=O)C3=O)c1-c1ccccc1C(F)(F)F ; Cn1ncc(-c2ccc3c(c2)CN(C2CCC(=O)NC2=O)C3=O)c1-c1cccc2ccccc12 ; Cn1ncc(-c2ccc3c(c2)CN(C2CCC(=O)NC2=O)C3=O)c1-c1ccc2ccccc2c1]

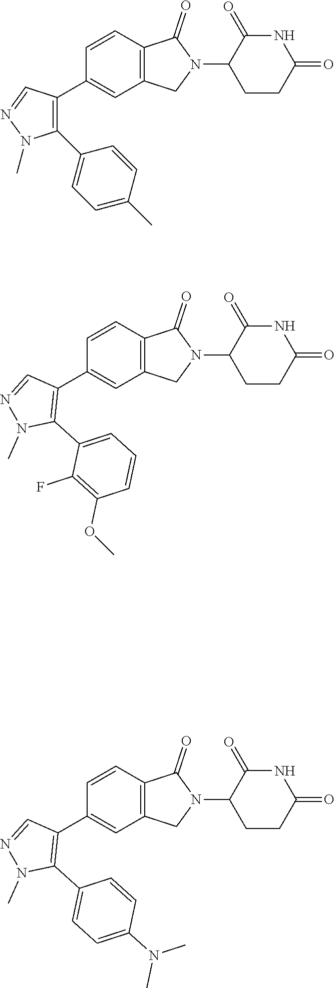 [SMILES: Cc1ccc(-c2c(-c3ccc4c(c3)CN(C3CCC(=O)NC3=O)C4=O)cnn2C)cc1 ; COc1cccc(-c2c(-c3ccc4c(c3)CN(C3CCC(=O)NC3=O)C4=O)cnn2C)c1F ; CN(C)c1ccc(-c2c(-c3ccc4c(c3)CN(C3CCC(=O)NC3=O)C4=O)cnn2C)cc1]

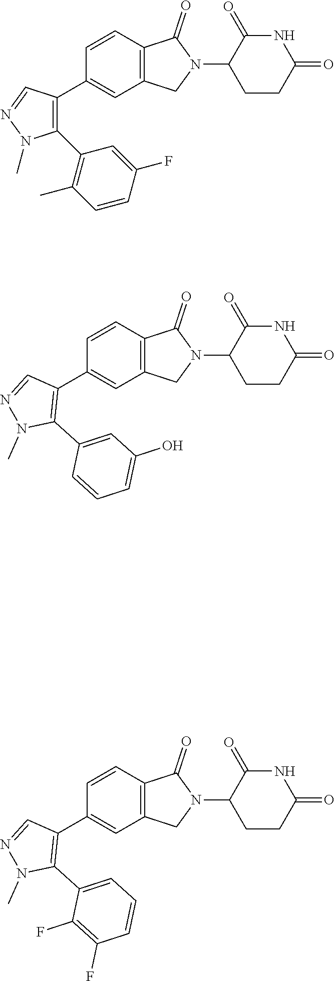 [SMILES: Cc1ccc(F)cc1-c1c(-c2ccc3c(c2)CN(C2CCC(=O)NC2=O)C3=O)cnn1C ; Cn1ncc(-c2ccc3c(c2)CN(C2CCC(=O)NC2=O)C3=O)c1-c1cccc(O)c1 ; Cn1ncc(-c2ccc3c(c2)CN(C2CCC(=O)NC2=O)C3=O)c1-c1cccc(F)c1F]

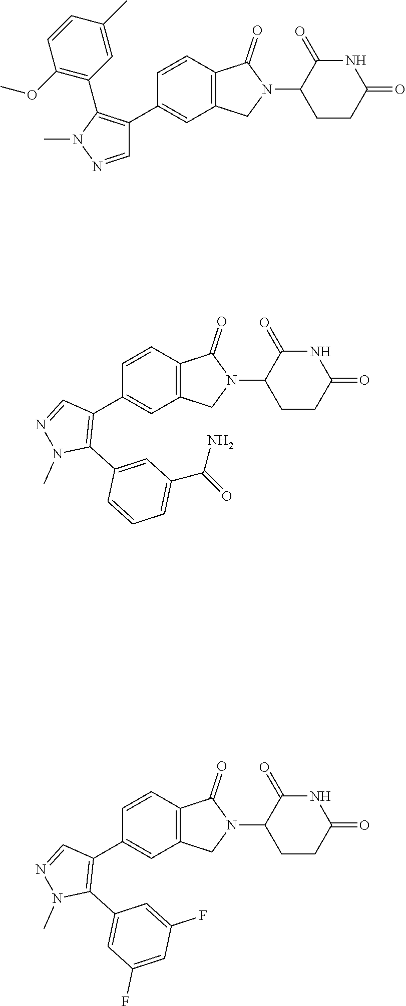 [SMILES: COc1ccc(C)cc1-c1c(-c2ccc3c(c2)CN(C2CCC(=O)NC2=O)C3=O)cnn1C ; Cn1ncc(-c2ccc3c(c2)CN(C2CCC(=O)NC2=O)C3=O)c1-c1cccc(C(N)=O)c1 ; Cn1ncc(-c2ccc3c(c2)CN(C2CCC(=O)NC2=O)C3=O)c1-c1cc(F)cc(F)c1]

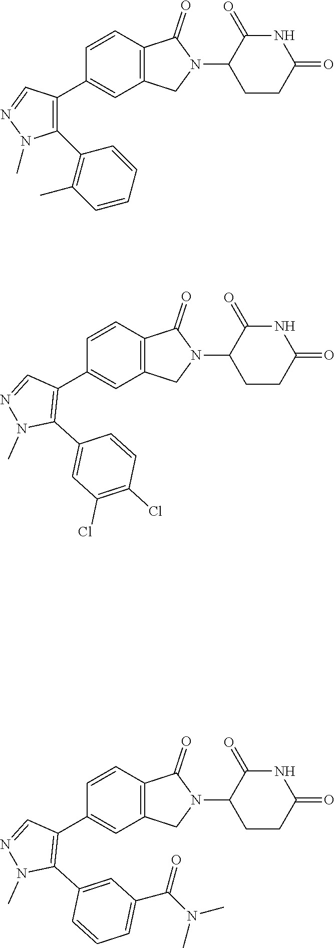 [SMILES: Cc1ccccc1-c1c(-c2ccc3c(c2)CN(C2CCC(=O)NC2=O)C3=O)cnn1C ; Cn1ncc(-c2ccc3c(c2)CN(C2CCC(=O)NC2=O)C3=O)c1-c1ccc(Cl)c(Cl)c1 ; CN(C)C(=O)c1cccc(-c2c(-c3ccc4c(c3)CN(C3CCC(=O)NC3=O)C4=O)cnn2C)c1]

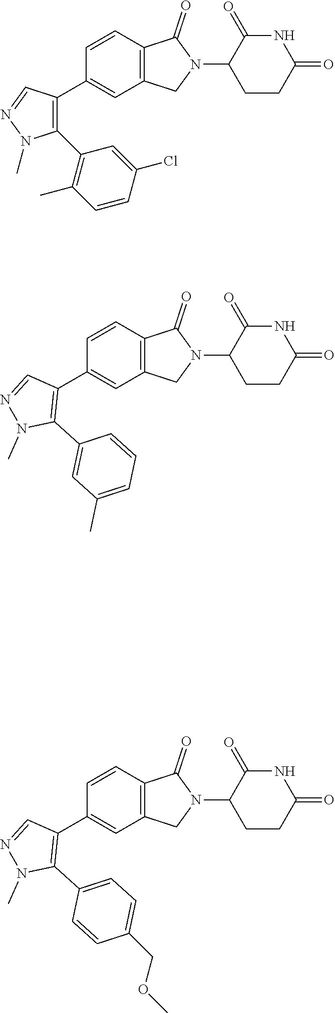 [SMILES: Cc1ccc(Cl)cc1-c1c(-c2ccc3c(c2)CN(C2CCC(=O)NC2=O)C3=O)cnn1C ; Cc1cccc(-c2c(-c3ccc4c(c3)CN(C3CCC(=O)NC3=O)C4=O)cnn2C)c1 ; COCc1ccc(-c2c(-c3ccc4c(c3)CN(C3CCC(=O)NC3=O)C4=O)cnn2C)cc1]

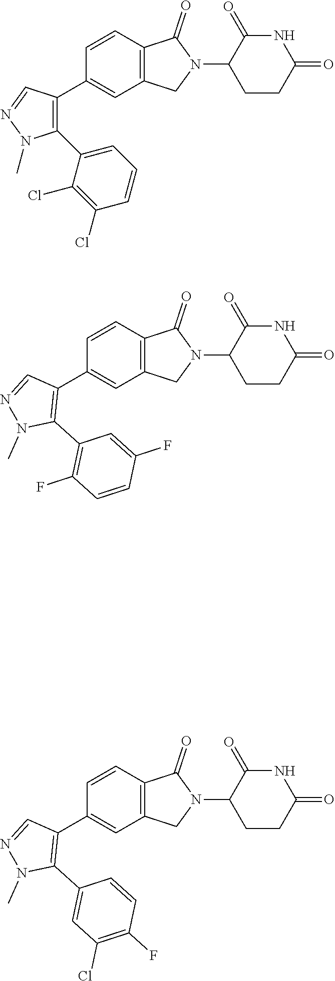 [SMILES: Cn1ncc(-c2ccc3c(c2)CN(C2CCC(=O)NC2=O)C3=O)c1-c1cccc(Cl)c1Cl ; Cn1ncc(-c2ccc3c(c2)CN(C2CCC(=O)NC2=O)C3=O)c1-c1cc(F)ccc1F ; Cn1ncc(-c2ccc3c(c2)CN(C2CCC(=O)NC2=O)C3=O)c1-c1ccc(F)c(Cl)c1]

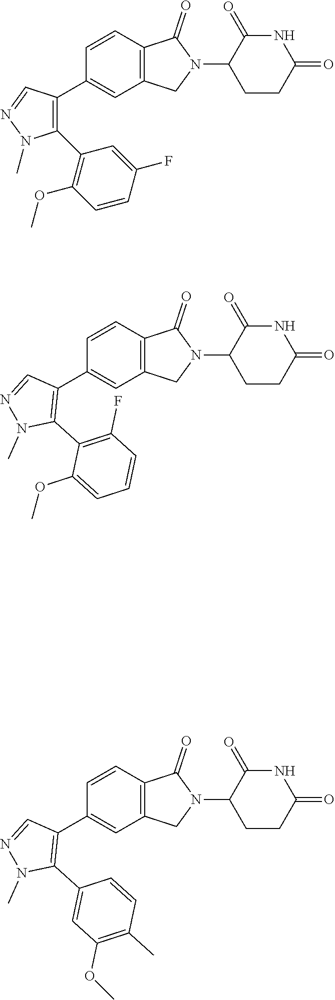 [SMILES: COc1ccc(F)cc1-c1c(-c2ccc3c(c2)CN(C2CCC(=O)NC2=O)C3=O)cnn1C ; COc1cccc(F)c1-c1c(-c2ccc3c(c2)CN(C2CCC(=O)NC2=O)C3=O)cnn1C ; COc1cc(-c2c(-c3ccc4c(c3)CN(C3CCC(=O)NC3=O)C4=O)cnn2C)ccc1C]

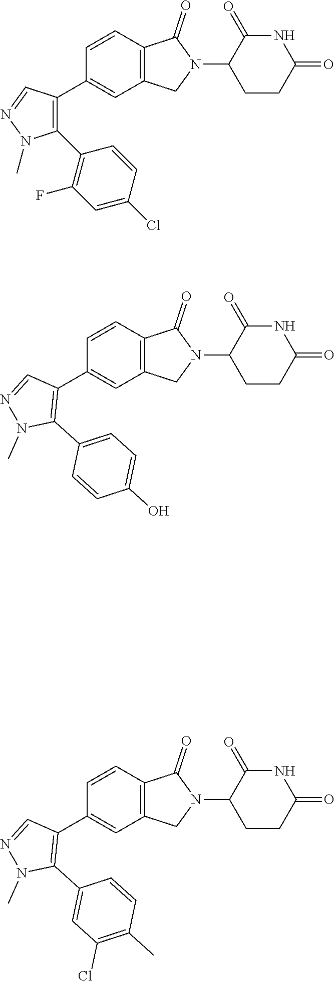 [SMILES: Cn1ncc(-c2ccc3c(c2)CN(C2CCC(=O)NC2=O)C3=O)c1-c1ccc(Cl)cc1F ; Cn1ncc(-c2ccc3c(c2)CN(C2CCC(=O)NC2=O)C3=O)c1-c1ccc(O)cc1 ; Cc1ccc(-c2c(-c3ccc4c(c3)CN(C3CCC(=O)NC3=O)C4=O)cnn2C)cc1Cl]

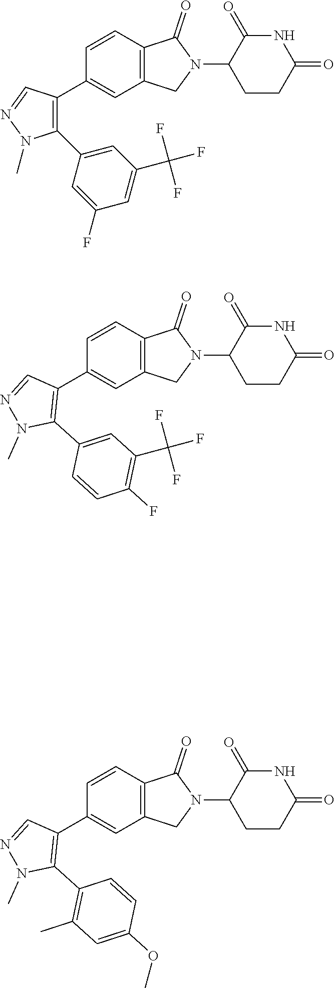 [SMILES: Cn1ncc(-c2ccc3c(c2)CN(C2CCC(=O)NC2=O)C3=O)c1-c1cc(F)cc(C(F)(F)F)c1 ; Cn1ncc(-c2ccc3c(c2)CN(C2CCC(=O)NC2=O)C3=O)c1-c1ccc(F)c(C(F)(F)F)c1 ; COc1ccc(-c2c(-c3ccc4c(c3)CN(C3CCC(=O)NC3=O)C4=O)cnn2C)c(C)c1]

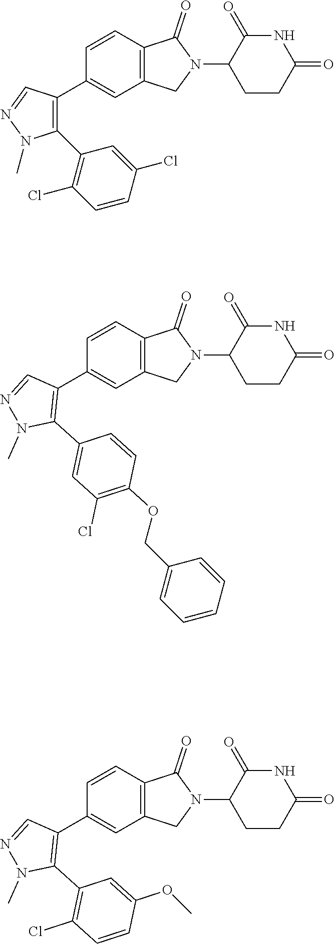 [SMILES: Cn1ncc(-c2ccc3c(c2)CN(C2CCC(=O)NC2=O)C3=O)c1-c1cc(Cl)ccc1Cl ; Cn1ncc(-c2ccc3c(c2)CN(C2CCC(=O)NC2=O)C3=O)c1-c1ccc(OCc2ccccc2)c(Cl)c1 ; COc1ccc(Cl)c(-c2c(-c3ccc4c(c3)CN(C3CCC(=O)NC3=O)C4=O)cnn2C)c1]

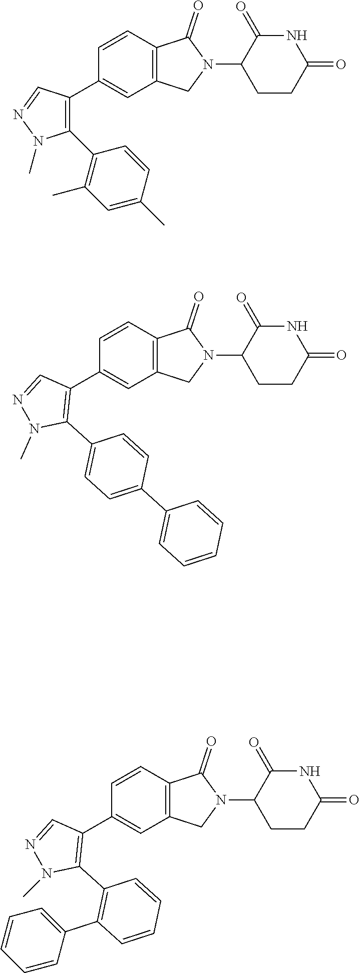 [SMILES: Cc1ccc(-c2c(-c3ccc4c(c3)CN(C3CCC(=O)NC3=O)C4=O)cnn2C)c(C)c1 ; Cn1ncc(-c2ccc3c(c2)CN(C2CCC(=O)NC2=O)C3=O)c1-c1ccc(-c2ccccc2)cc1 ; Cn1ncc(-c2ccc3c(c2)CN(C2CCC(=O)NC2=O)C3=O)c1-c1ccccc1-c1ccccc1]

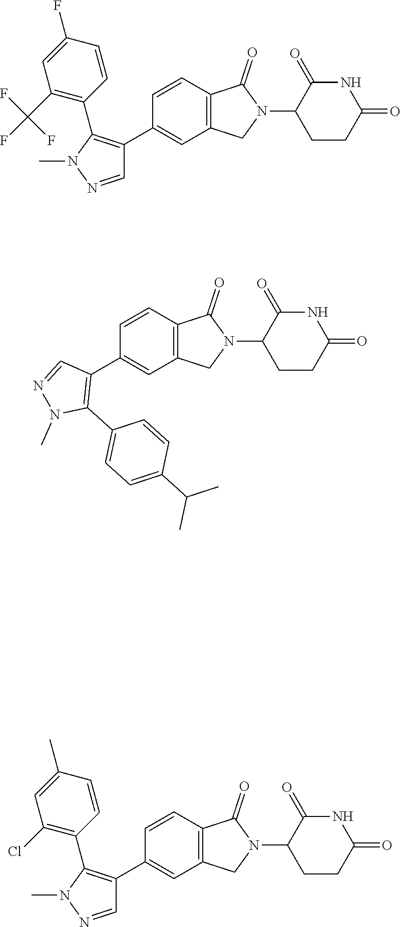 [SMILES: Cn1ncc(-c2ccc3c(c2)CN(C2CCC(=O)NC2=O)C3=O)c1-c1ccc(F)cc1C(F)(F)F ; CC(C)c1ccc(-c2c(-c3ccc4c(c3)CN(C3CCC(=O)NC3=O)C4=O)cnn2C)cc1 ; Cc1ccc(-c2c(-c3ccc4c(c3)CN(C3CCC(=O)NC3=O)C4=O)cnn2C)c(Cl)c1]

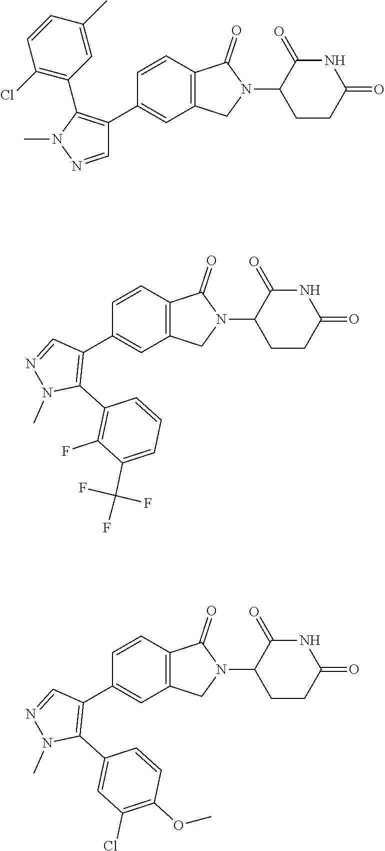 [SMILES: Cc1ccc(Cl)c(-c2c(-c3ccc4c(c3)CN(C3CCC(=O)NC3=O)C4=O)cnn2C)c1 ; Cn1ncc(-c2ccc3c(c2)CN(C2CCC(=O)NC2=O)C3=O)c1-c1cccc(C(F)(F)F)c1F ; COc1ccc(-c2c(-c3ccc4c(c3)CN(C3CCC(=O)NC3=O)C4=O)cnn2C)cc1Cl]

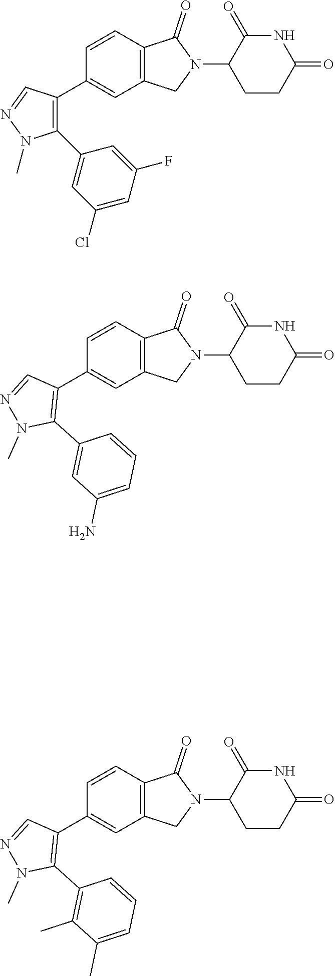 [SMILES: Cn1ncc(-c2ccc3c(c2)CN(C2CCC(=O)NC2=O)C3=O)c1-c1cc(F)cc(Cl)c1 ; Cn1ncc(-c2ccc3c(c2)CN(C2CCC(=O)NC2=O)C3=O)c1-c1cccc(N)c1 ; Cc1cccc(-c2c(-c3ccc4c(c3)CN(C3CCC(=O)NC3=O)C4=O)cnn2C)c1C]

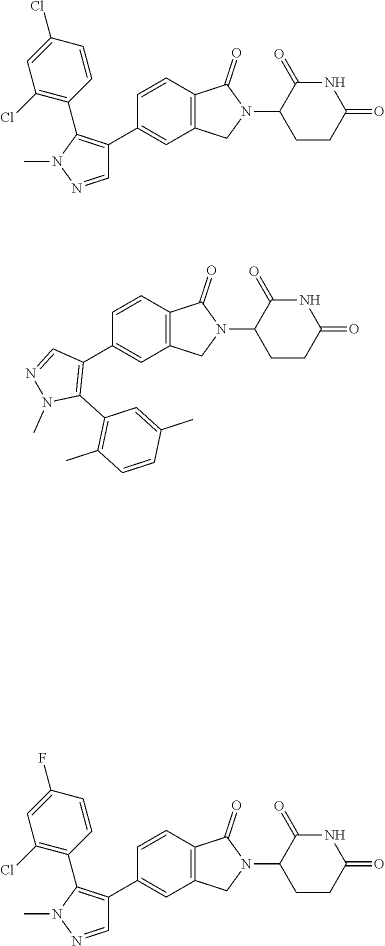 [SMILES: Cn1ncc(-c2ccc3c(c2)CN(C2CCC(=O)NC2=O)C3=O)c1-c1ccc(Cl)cc1Cl ; Cc1ccc(C)c(-c2c(-c3ccc4c(c3)CN(C3CCC(=O)NC3=O)C4=O)cnn2C)c1 ; Cn1ncc(-c2ccc3c(c2)CN(C2CCC(=O)NC2=O)C3=O)c1-c1ccc(F)cc1Cl]

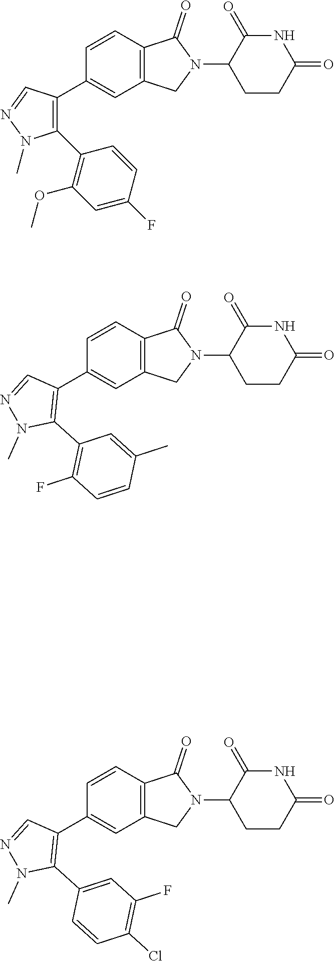 [SMILES: COc1cc(F)ccc1-c1c(-c2ccc3c(c2)CN(C2CCC(=O)NC2=O)C3=O)cnn1C ; Cc1ccc(F)c(-c2c(-c3ccc4c(c3)CN(C3CCC(=O)NC3=O)C4=O)cnn2C)c1 ; Cn1ncc(-c2ccc3c(c2)CN(C2CCC(=O)NC2=O)C3=O)c1-c1ccc(Cl)c(F)c1]

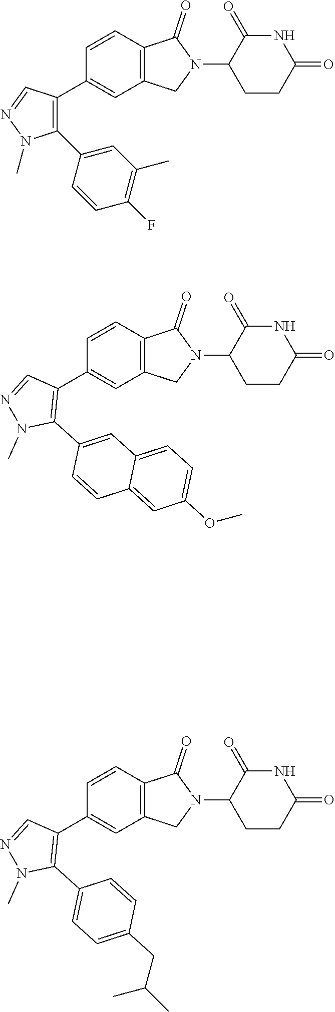 [SMILES: Cc1cc(-c2c(-c3ccc4c(c3)CN(C3CCC(=O)NC3=O)C4=O)cnn2C)ccc1F ; COc1ccc2cc(-c3c(-c4ccc5c(c4)CN(C4CCC(=O)NC4=O)C5=O)cnn3C)ccc2c1 ; CC(C)Cc1ccc(-c2c(-c3ccc4c(c3)CN(C3CCC(=O)NC3=O)C4=O)cnn2C)cc1]

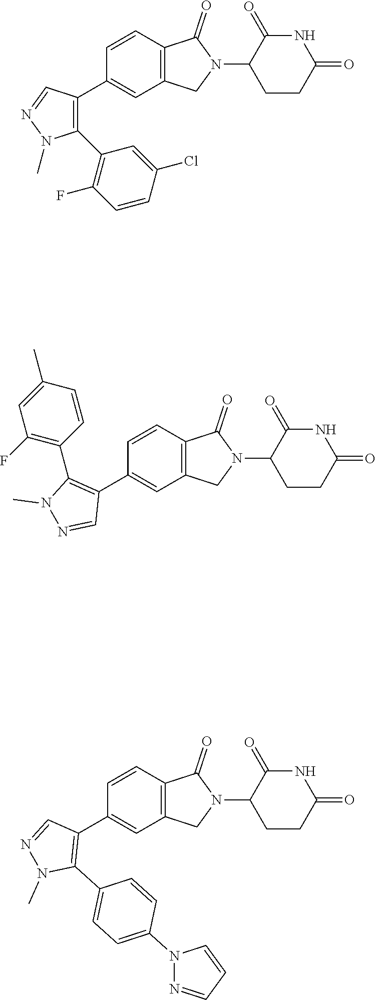 [SMILES: Cn1ncc(-c2ccc3c(c2)CN(C2CCC(=O)NC2=O)C3=O)c1-c1cc(Cl)ccc1F ; Cc1ccc(-c2c(-c3ccc4c(c3)CN(C3CCC(=O)NC3=O)C4=O)cnn2C)c(F)c1 ; Cn1ncc(-c2ccc3c(c2)CN(C2CCC(=O)NC2=O)C3=O)c1-c1ccc(-n2cccn2)cc1]

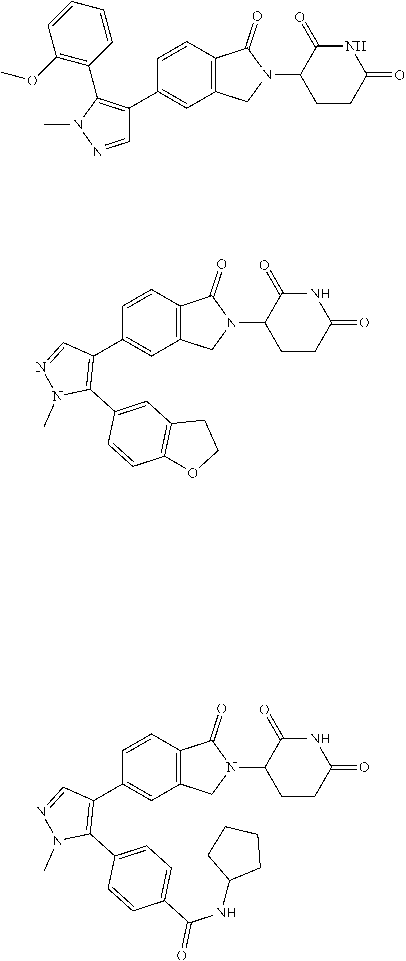 [SMILES: COc1ccccc1-c1c(-c2ccc3c(c2)CN(C2CCC(=O)NC2=O)C3=O)cnn1C ; Cn1ncc(-c2ccc3c(c2)CN(C2CCC(=O)NC2=O)C3=O)c1-c1ccc2c(c1)CCO2 ; Cn1ncc(-c2ccc3c(c2)CN(C2CCC(=O)NC2=O)C3=O)c1-c1ccc(C(=O)NC2CCCC2)cc1]

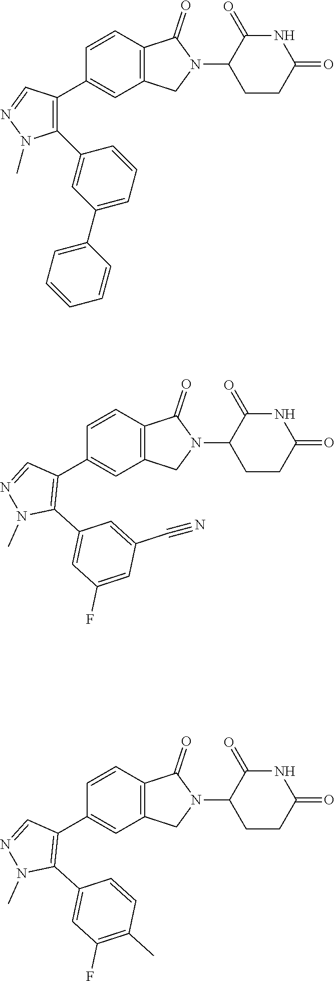 [SMILES: Cn1ncc(-c2ccc3c(c2)CN(C2CCC(=O)NC2=O)C3=O)c1-c1cccc(-c2ccccc2)c1 ; Cn1ncc(-c2ccc3c(c2)CN(C2CCC(=O)NC2=O)C3=O)c1-c1cc(F)cc(C#N)c1 ; Cc1ccc(-c2c(-c3ccc4c(c3)CN(C3CCC(=O)NC3=O)C4=O)cnn2C)cc1F]

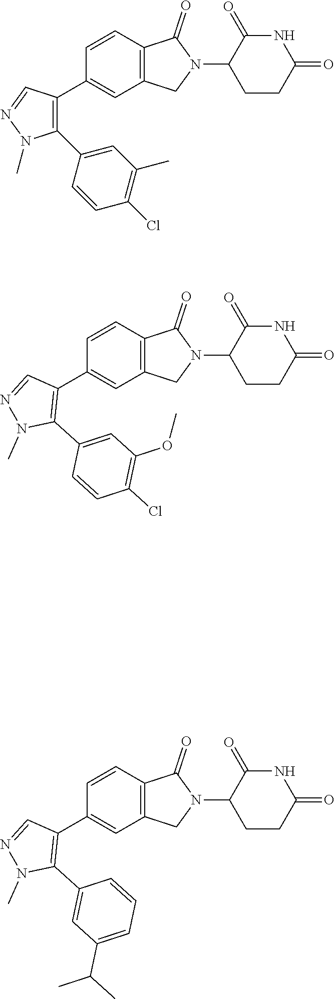 [SMILES: Cc1cc(-c2c(-c3ccc4c(c3)CN(C3CCC(=O)NC3=O)C4=O)cnn2C)ccc1Cl ; COc1cc(-c2c(-c3ccc4c(c3)CN(C3CCC(=O)NC3=O)C4=O)cnn2C)ccc1Cl ; CC(C)c1cccc(-c2c(-c3ccc4c(c3)CN(C3CCC(=O)NC3=O)C4=O)cnn2C)c1]

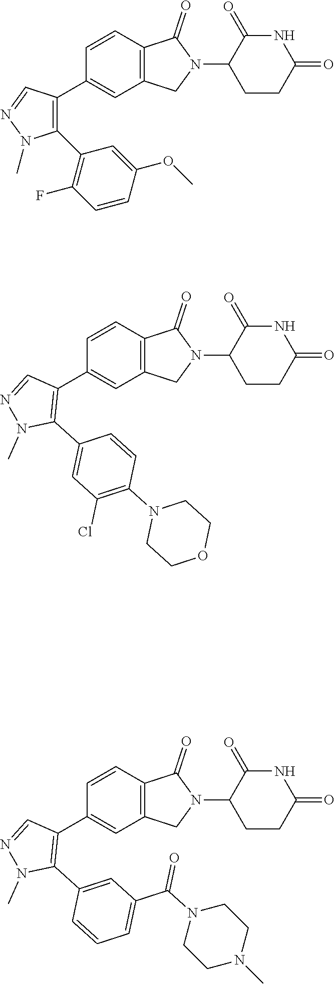 [SMILES: COc1ccc(F)c(-c2c(-c3ccc4c(c3)CN(C3CCC(=O)NC3=O)C4=O)cnn2C)c1 ; Cn1ncc(-c2ccc3c(c2)CN(C2CCC(=O)NC2=O)C3=O)c1-c1ccc(N2CCOCC2)c(Cl)c1 ; CN1CCN(C(=O)c2cccc(-c3c(-c4ccc5c(c4)CN(C4CCC(=O)NC4=O)C5=O)cnn3C)c2)CC1]

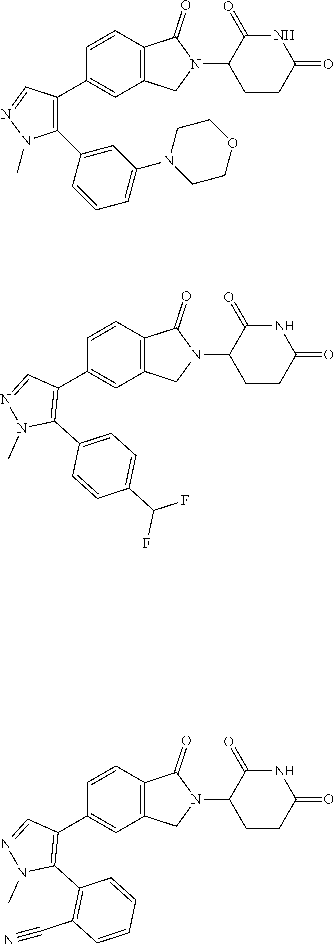 [SMILES: Cn1ncc(-c2ccc3c(c2)CN(C2CCC(=O)NC2=O)C3=O)c1-c1cccc(N2CCOCC2)c1 ; Cn1ncc(-c2ccc3c(c2)CN(C2CCC(=O)NC2=O)C3=O)c1-c1ccc(C(F)F)cc1 ; Cn1ncc(-c2ccc3c(c2)CN(C2CCC(=O)NC2=O)C3=O)c1-c1ccccc1C#N]

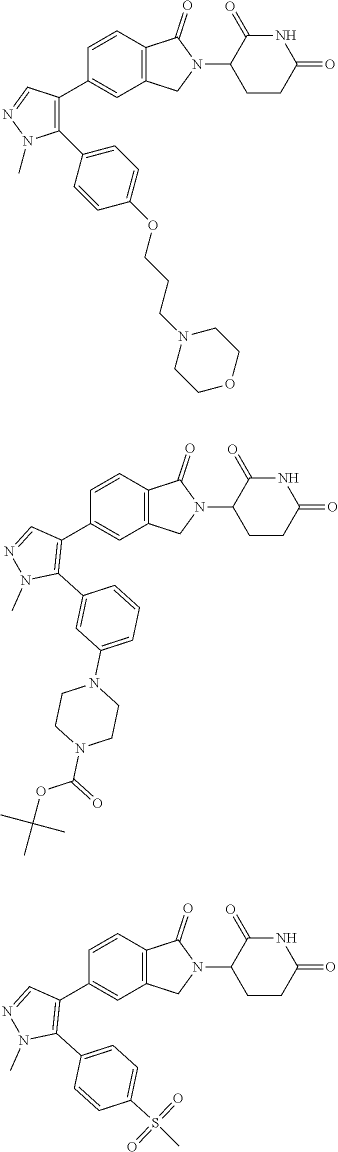 [SMILES: Cn1ncc(-c2ccc3c(c2)CN(C2CCC(=O)NC2=O)C3=O)c1-c1ccc(OCCCN2CCOCC2)cc1 ; Cn1ncc(-c2ccc3c(c2)CN(C2CCC(=O)NC2=O)C3=O)c1-c1cccc(N2CCN(C(=O)OC(C)(C)C)CC2)c1 ; Cn1ncc(-c2ccc3c(c2)CN(C2CCC(=O)NC2=O)C3=O)c1-c1ccc(S(C)(=O)=O)cc1]

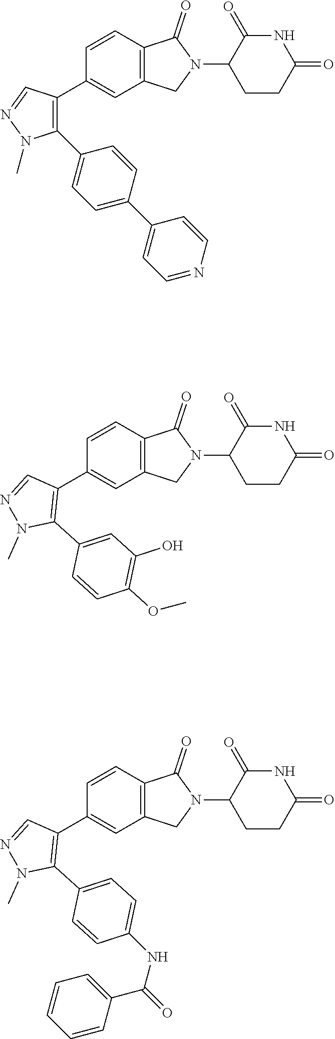 [SMILES: Cn1ncc(-c2ccc3c(c2)CN(C2CCC(=O)NC2=O)C3=O)c1-c1ccc(-c2ccncc2)cc1 ; COc1ccc(-c2c(-c3ccc4c(c3)CN(C3CCC(=O)NC3=O)C4=O)cnn2C)cc1O ; Cn1ncc(-c2ccc3c(c2)CN(C2CCC(=O)NC2=O)C3=O)c1-c1ccc(NC(=O)c2ccccc2)cc1]

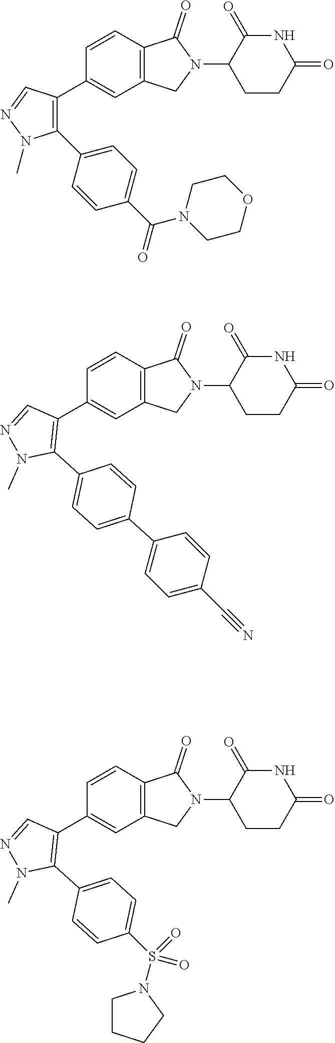 [SMILES: Cn1ncc(-c2ccc3c(c2)CN(C2CCC(=O)NC2=O)C3=O)c1-c1ccc(C(=O)N2CCOCC2)cc1 ; Cn1ncc(-c2ccc3c(c2)CN(C2CCC(=O)NC2=O)C3=O)c1-c1ccc(-c2ccc(C#N)cc2)cc1 ; Cn1ncc(-c2ccc3c(c2)CN(C2CCC(=O)NC2=O)C3=O)c1-c1ccc(S(=O)(=O)N2CCCC2)cc1]

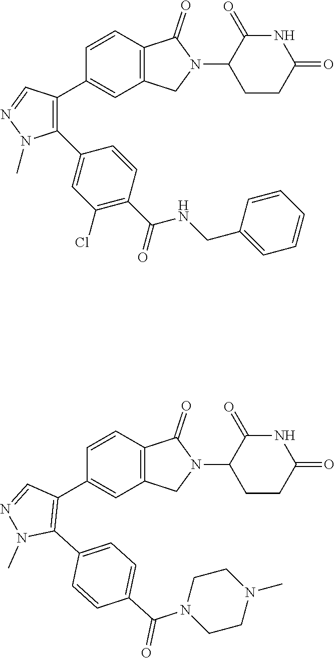 [SMILES: Cn1ncc(-c2ccc3c(c2)CN(C2CCC(=O)NC2=O)C3=O)c1-c1ccc(C(=O)NCc2ccccc2)c(Cl)c1 ; CN1CCN(C(=O)c2ccc(-c3c(-c4ccc5c(c4)CN(C4CCC(=O)NC4=O)C5=O)cnn3C)cc2)CC1]

In another embodiment, the compound provided herein for use in the compositions and methods provided herein is selected from:

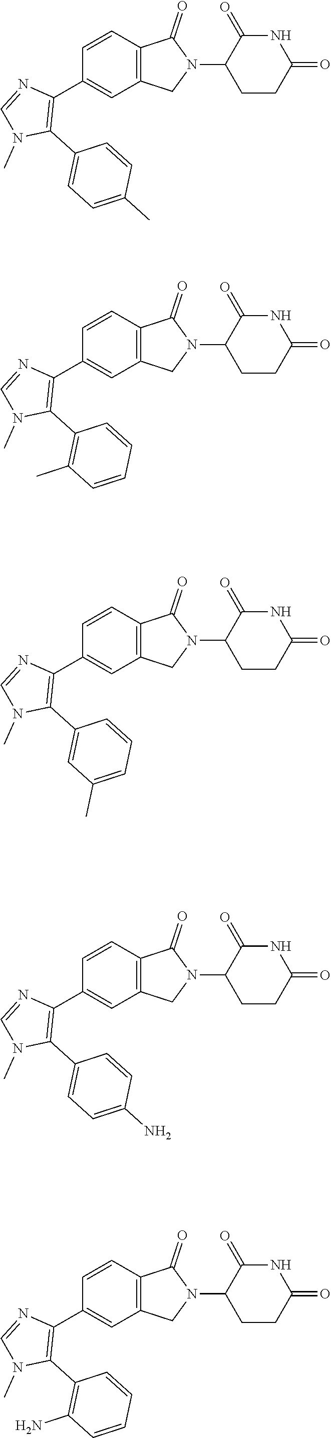 [SMILES: Cc1ccc(-c2c(-c3ccc4c(c3)CN(C3CCC(=O)NC3=O)C4=O)ncn2C)cc1 ; Cc1ccccc1-c1c(-c2ccc3c(c2)CN(C2CCC(=O)NC2=O)C3=O)ncn1C ; Cc1cccc(-c2c(-c3ccc4c(c3)CN(C3CCC(=O)NC3=O)C4=O)ncn2C)c1 ; Cn1cnc(-c2ccc3c(c2)CN(C2CCC(=O)NC2=O)C3=O)c1-c1ccc(N)cc1 ; Cn1cnc(-c2ccc3c(c2)CN(C2CCC(=O)NC2=O)C3=O)c1-c1ccccc1N]

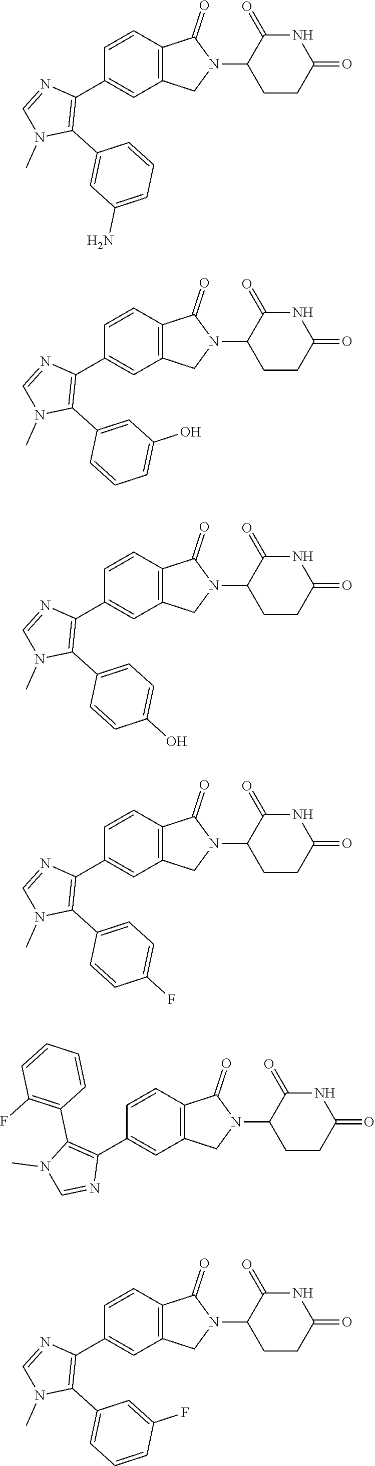 [SMILES: Cn1cnc(-c2ccc3c(c2)CN(C2CCC(=O)NC2=O)C3=O)c1-c1cccc(N)c1 ; Cn1cnc(-c2ccc3c(c2)CN(C2CCC(=O)NC2=O)C3=O)c1-c1cccc(O)c1 ; Cn1cnc(-c2ccc3c(c2)CN(C2CCC(=O)NC2=O)C3=O)c1-c1ccc(O)cc1 ; Cn1cnc(-c2ccc3c(c2)CN(C2CCC(=O)NC2=O)C3=O)c1-c1ccc(F)cc1 ; Cn1cnc(-c2ccc3c(c2)CN(C2CCC(=O)NC2=O)C3=O)c1-c1ccccc1F ; Cn1cnc(-c2ccc3c(c2)CN(C2CCC(=O)NC2=O)C3=O)c1-c1cccc(F)c1]

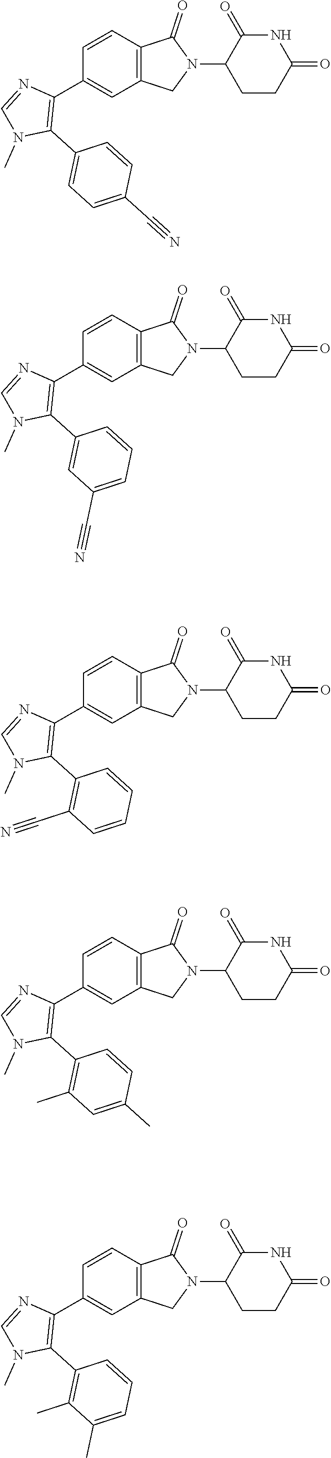 [SMILES: Cn1cnc(-c2ccc3c(c2)CN(C2CCC(=O)NC2=O)C3=O)c1-c1ccc(C#N)cc1 ; Cn1cnc(-c2ccc3c(c2)CN(C2CCC(=O)NC2=O)C3=O)c1-c1cccc(C#N)c1 ; Cn1cnc(-c2ccc3c(c2)CN(C2CCC(=O)NC2=O)C3=O)c1-c1ccccc1C#N ; Cc1ccc(-c2c(-c3ccc4c(c3)CN(C3CCC(=O)NC3=O)C4=O)ncn2C)c(C)c1 ; Cc1cccc(-c2c(-c3ccc4c(c3)CN(C3CCC(=O)NC3=O)C4=O)ncn2C)c1C]

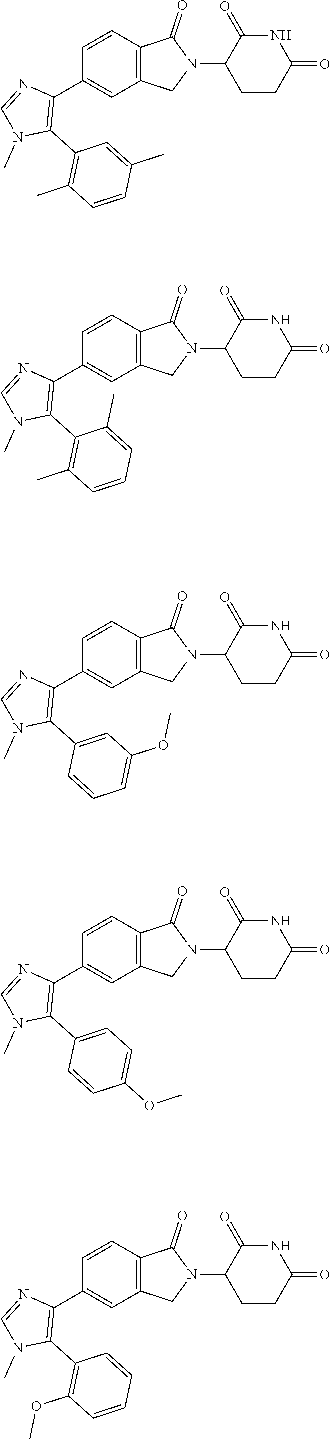 [SMILES: Cc1ccc(C)c(-c2c(-c3ccc4c(c3)CN(C3CCC(=O)NC3=O)C4=O)ncn2C)c1 ; Cc1cccc(C)c1-c1c(-c2ccc3c(c2)CN(C2CCC(=O)NC2=O)C3=O)ncn1C ; COc1cccc(-c2c(-c3ccc4c(c3)CN(C3CCC(=O)NC3=O)C4=O)ncn2C)c1 ; COc1ccc(-c2c(-c3ccc4c(c3)CN(C3CCC(=O)NC3=O)C4=O)ncn2C)cc1 ; COc1ccccc1-c1c(-c2ccc3c(c2)CN(C2CCC(=O)NC2=O)C3=O)ncn1C]

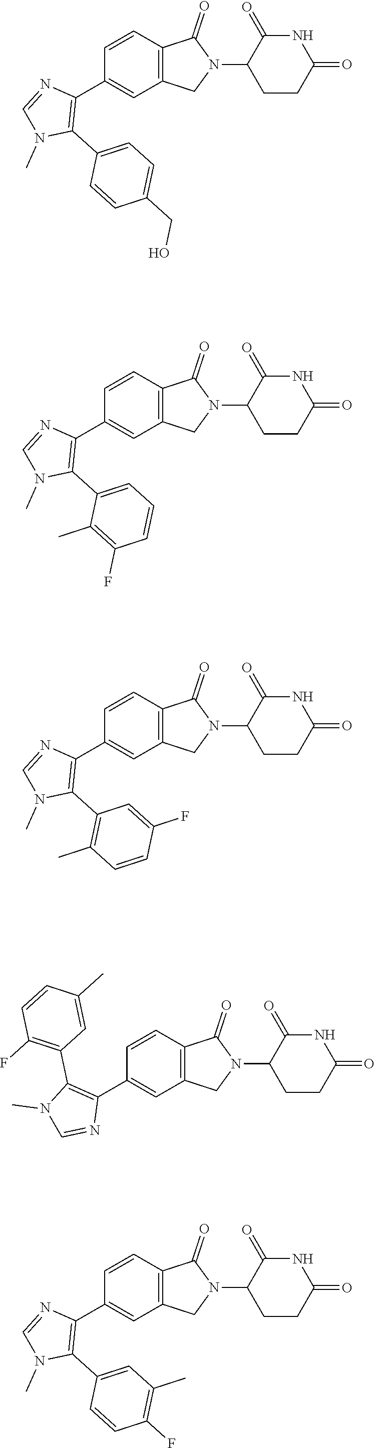 [SMILES: Cn1cnc(-c2ccc3c(c2)CN(C2CCC(=O)NC2=O)C3=O)c1-c1ccc(CO)cc1 ; Cc1c(F)cccc1-c1c(-c2ccc3c(c2)CN(C2CCC(=O)NC2=O)C3=O)ncn1C ; Cc1ccc(F)cc1-c1c(-c2ccc3c(c2)CN(C2CCC(=O)NC2=O)C3=O)ncn1C ; Cc1ccc(F)c(-c2c(-c3ccc4c(c3)CN(C3CCC(=O)NC3=O)C4=O)ncn2C)c1 ; Cc1cc(-c2c(-c3ccc4c(c3)CN(C3CCC(=O)NC3=O)C4=O)ncn2C)ccc1F]

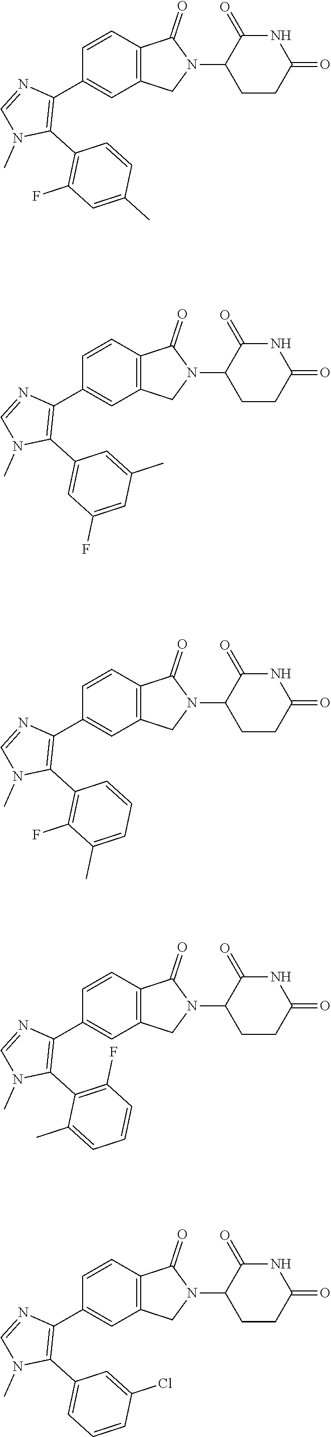 [SMILES: Cc1ccc(-c2c(-c3ccc4c(c3)CN(C3CCC(=O)NC3=O)C4=O)ncn2C)c(F)c1 ; Cc1cc(F)cc(-c2c(-c3ccc4c(c3)CN(C3CCC(=O)NC3=O)C4=O)ncn2C)c1 ; Cc1cccc(-c2c(-c3ccc4c(c3)CN(C3CCC(=O)NC3=O)C4=O)ncn2C)c1F ; Cc1cccc(F)c1-c1c(-c2ccc3c(c2)CN(C2CCC(=O)NC2=O)C3=O)ncn1C ; Cn1cnc(-c2ccc3c(c2)CN(C2CCC(=O)NC2=O)C3=O)c1-c1cccc(Cl)c1]

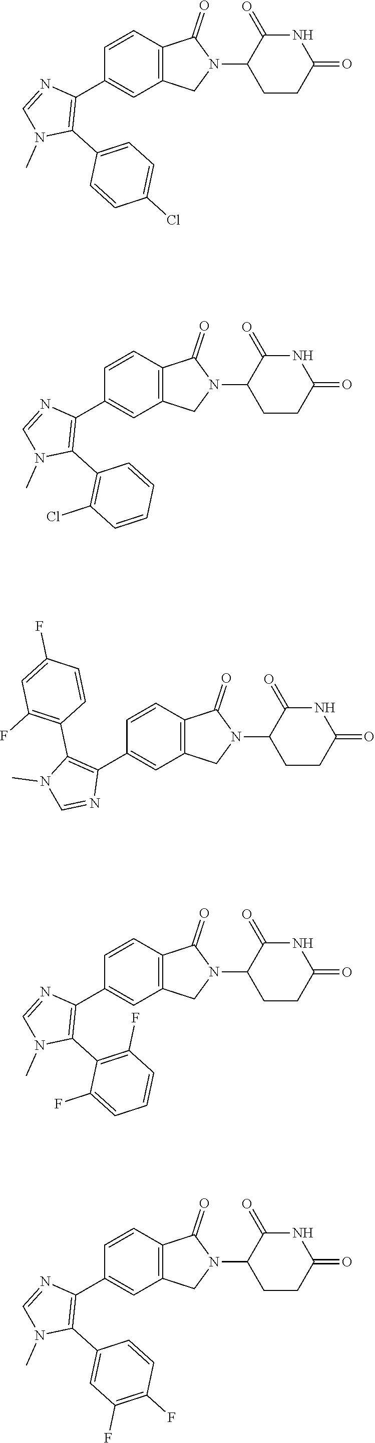 [SMILES: Cn1cnc(-c2ccc3c(c2)CN(C2CCC(=O)NC2=O)C3=O)c1-c1ccc(Cl)cc1 ; Cn1cnc(-c2ccc3c(c2)CN(C2CCC(=O)NC2=O)C3=O)c1-c1ccccc1Cl ; Cn1cnc(-c2ccc3c(c2)CN(C2CCC(=O)NC2=O)C3=O)c1-c1ccc(F)cc1F ; Cn1cnc(-c2ccc3c(c2)CN(C2CCC(=O)NC2=O)C3=O)c1-c1c(F)cccc1F ; Cn1cnc(-c2ccc3c(c2)CN(C2CCC(=O)NC2=O)C3=O)c1-c1ccc(F)c(F)c1]

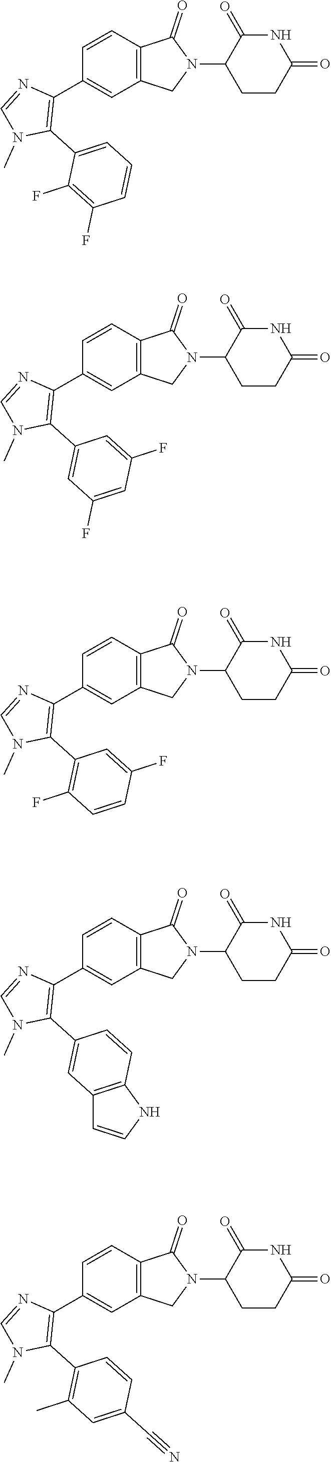 [SMILES: Cn1cnc(-c2ccc3c(c2)CN(C2CCC(=O)NC2=O)C3=O)c1-c1cccc(F)c1F ; Cn1cnc(-c2ccc3c(c2)CN(C2CCC(=O)NC2=O)C3=O)c1-c1cc(F)cc(F)c1 ; Cn1cnc(-c2ccc3c(c2)CN(C2CCC(=O)NC2=O)C3=O)c1-c1cc(F)ccc1F ; Cn1cnc(-c2ccc3c(c2)CN(C2CCC(=O)NC2=O)C3=O)c1-c1ccc2[nH]ccc2c1 ; Cc1cc(C#N)ccc1-c1c(-c2ccc3c(c2)CN(C2CCC(=O)NC2=O)C3=O)ncn1C]

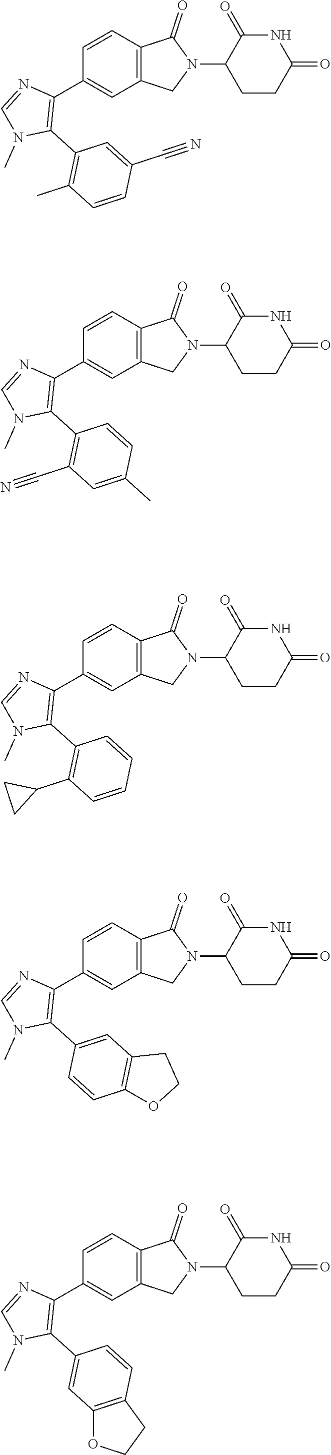 [SMILES: Cc1ccc(C#N)cc1-c1c(-c2ccc3c(c2)CN(C2CCC(=O)NC2=O)C3=O)ncn1C ; Cc1ccc(-c2c(-c3ccc4c(c3)CN(C3CCC(=O)NC3=O)C4=O)ncn2C)c(C#N)c1 ; Cn1cnc(-c2ccc3c(c2)CN(C2CCC(=O)NC2=O)C3=O)c1-c1ccccc1C1CC1 ; Cn1cnc(-c2ccc3c(c2)CN(C2CCC(=O)NC2=O)C3=O)c1-c1ccc2c(c1)CCO2 ; Cn1cnc(-c2ccc3c(c2)CN(C2CCC(=O)NC2=O)C3=O)c1-c1ccc2c(c1)OCC2]

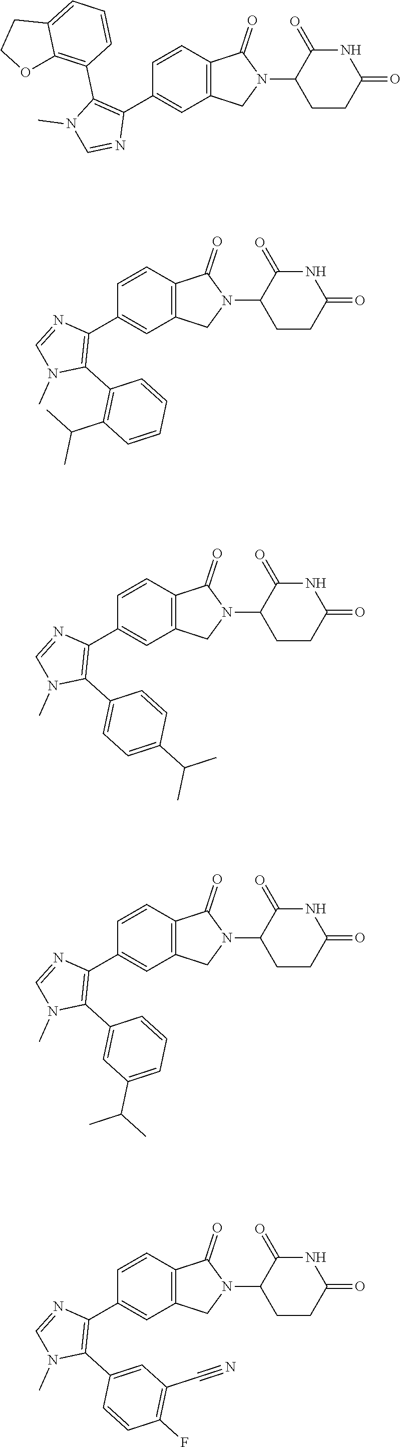 [SMILES: Cn1cnc(-c2ccc3c(c2)CN(C2CCC(=O)NC2=O)C3=O)c1-c1cccc2c1OCC2 ; CC(C)c1ccccc1-c1c(-c2ccc3c(c2)CN(C2CCC(=O)NC2=O)C3=O)ncn1C ; CC(C)c1ccc(-c2c(-c3ccc4c(c3)CN(C3CCC(=O)NC3=O)C4=O)ncn2C)cc1 ; CC(C)c1cccc(-c2c(-c3ccc4c(c3)CN(C3CCC(=O)NC3=O)C4=O)ncn2C)c1 ; Cn1cnc(-c2ccc3c(c2)CN(C2CCC(=O)NC2=O)C3=O)c1-c1ccc(F)c(C#N)c1]

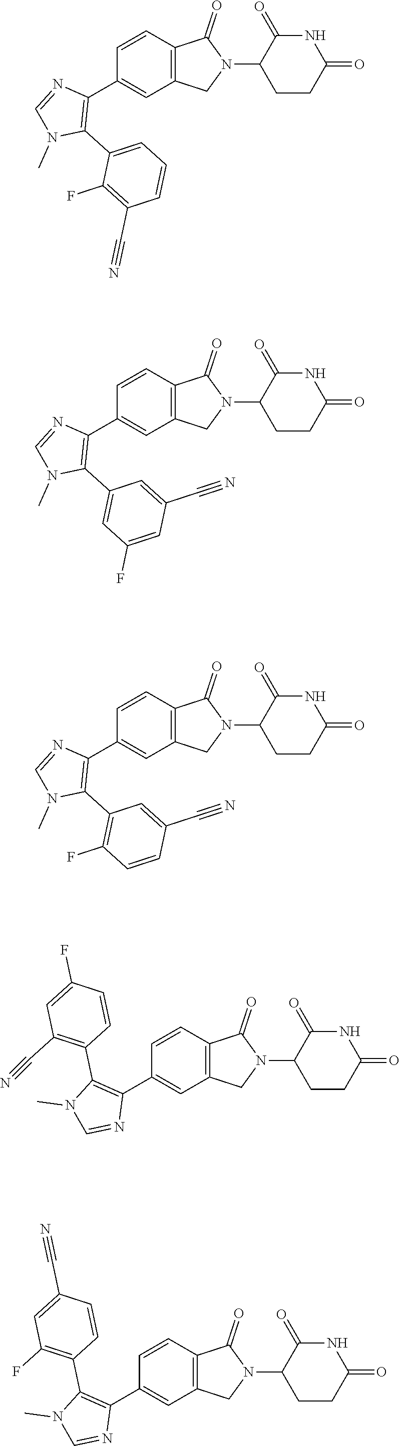 [SMILES: Cn1cnc(-c2ccc3c(c2)CN(C2CCC(=O)NC2=O)C3=O)c1-c1cccc(C#N)c1F ; Cn1cnc(-c2ccc3c(c2)CN(C2CCC(=O)NC2=O)C3=O)c1-c1cc(F)cc(C#N)c1 ; Cn1cnc(-c2ccc3c(c2)CN(C2CCC(=O)NC2=O)C3=O)c1-c1cc(C#N)ccc1F ; Cn1cnc(-c2ccc3c(c2)CN(C2CCC(=O)NC2=O)C3=O)c1-c1ccc(F)cc1C#N ; Cn1cnc(-c2ccc3c(c2)CN(C2CCC(=O)NC2=O)C3=O)c1-c1ccc(C#N)cc1F]

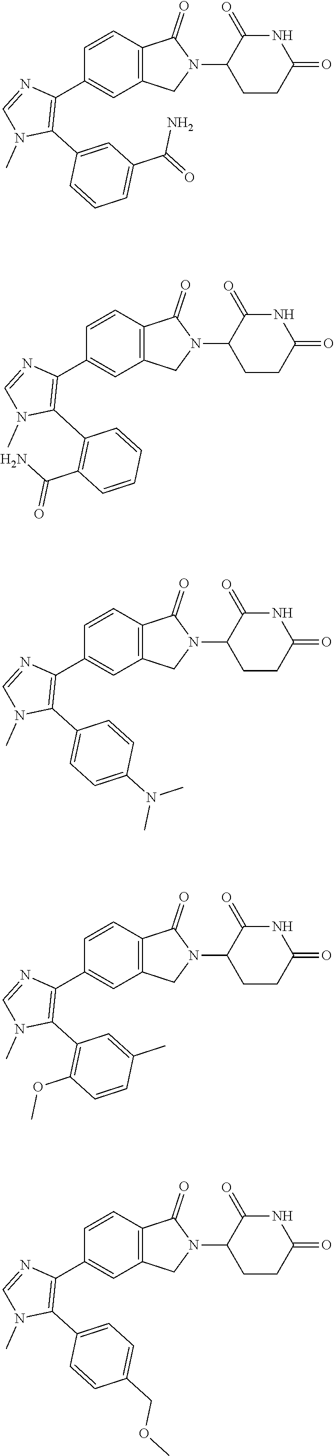 [SMILES: Cn1cnc(-c2ccc3c(c2)CN(C2CCC(=O)NC2=O)C3=O)c1-c1cccc(C(N)=O)c1 ; Cn1cnc(-c2ccc3c(c2)CN(C2CCC(=O)NC2=O)C3=O)c1-c1ccccc1C(N)=O ; CN(C)c1ccc(-c2c(-c3ccc4c(c3)CN(C3CCC(=O)NC3=O)C4=O)ncn2C)cc1 ; COc1ccc(C)cc1-c1c(-c2ccc3c(c2)CN(C2CCC(=O)NC2=O)C3=O)ncn1C ; COCc1ccc(-c2c(-c3ccc4c(c3)CN(C3CCC(=O)NC3=O)C4=O)ncn2C)cc1]

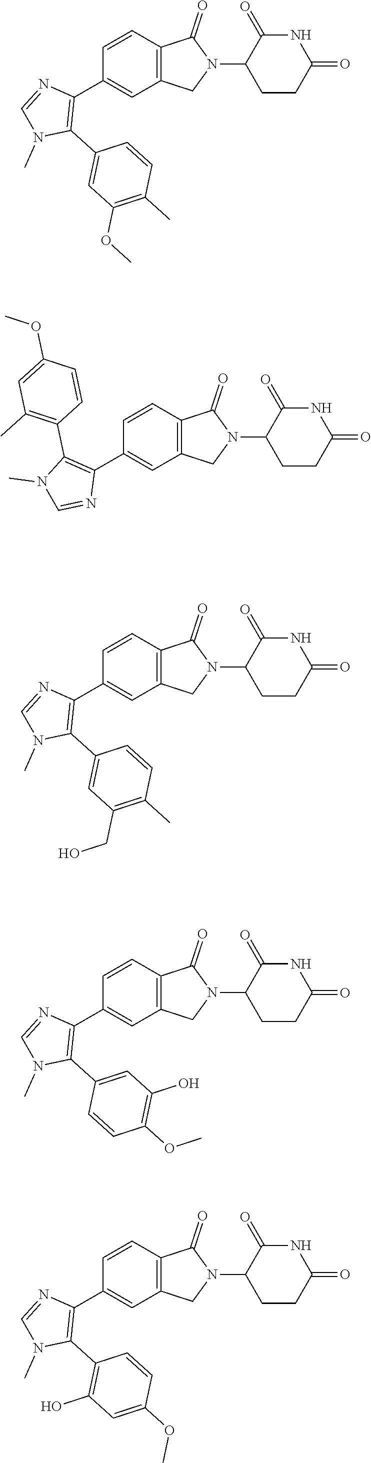 [SMILES: COc1cc(-c2c(-c3ccc4c(c3)CN(C3CCC(=O)NC3=O)C4=O)ncn2C)ccc1C ; COc1ccc(-c2c(-c3ccc4c(c3)CN(C3CCC(=O)NC3=O)C4=O)ncn2C)c(C)c1 ; Cc1ccc(-c2c(-c3ccc4c(c3)CN(C3CCC(=O)NC3=O)C4=O)ncn2C)cc1CO ; COc1ccc(-c2c(-c3ccc4c(c3)CN(C3CCC(=O)NC3=O)C4=O)ncn2C)cc1O ; COc1ccc(-c2c(-c3ccc4c(c3)CN(C3CCC(=O)NC3=O)C4=O)ncn2C)c(O)c1]

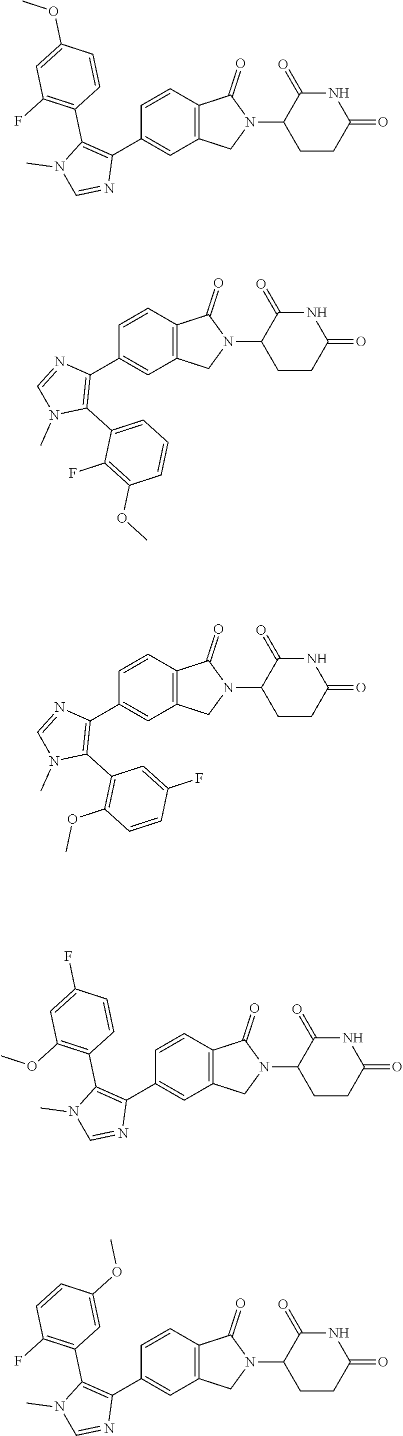 [SMILES: COc1ccc(-c2c(-c3ccc4c(c3)CN(C3CCC(=O)NC3=O)C4=O)ncn2C)c(F)c1 ; COc1cccc(-c2c(-c3ccc4c(c3)CN(C3CCC(=O)NC3=O)C4=O)ncn2C)c1F ; COc1ccc(F)cc1-c1c(-c2ccc3c(c2)CN(C2CCC(=O)NC2=O)C3=O)ncn1C ; COc1cc(F)ccc1-c1c(-c2ccc3c(c2)CN(C2CCC(=O)NC2=O)C3=O)ncn1C ; COc1ccc(F)c(-c2c(-c3ccc4c(c3)CN(C3CCC(=O)NC3=O)C4=O)ncn2C)c1]

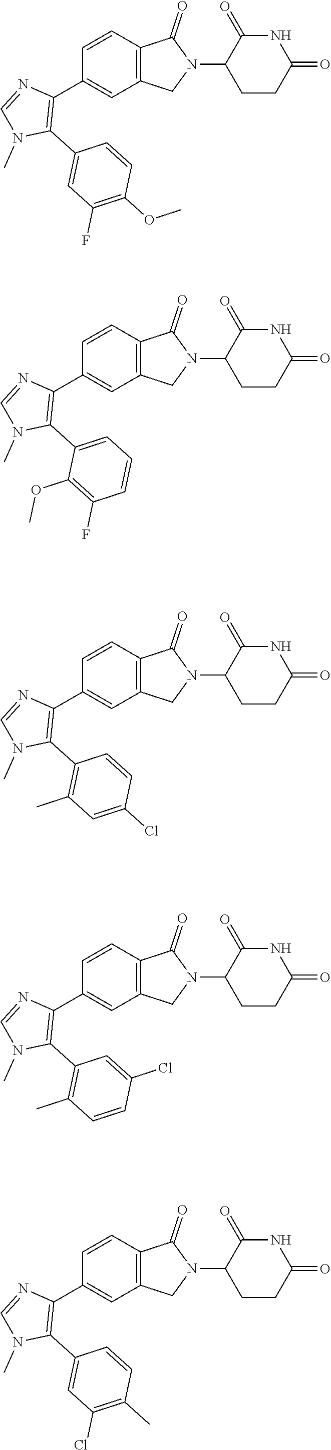 [SMILES: COc1ccc(-c2c(-c3ccc4c(c3)CN(C3CCC(=O)NC3=O)C4=O)ncn2C)cc1F ; COc1c(F)cccc1-c1c(-c2ccc3c(c2)CN(C2CCC(=O)NC2=O)C3=O)ncn1C ; Cc1cc(Cl)ccc1-c1c(-c2ccc3c(c2)CN(C2CCC(=O)NC2=O)C3=O)ncn1C ; Cc1ccc(Cl)cc1-c1c(-c2ccc3c(c2)CN(C2CCC(=O)NC2=O)C3=O)ncn1C ; Cc1ccc(-c2c(-c3ccc4c(c3)CN(C3CCC(=O)NC3=O)C4=O)ncn2C)cc1Cl]

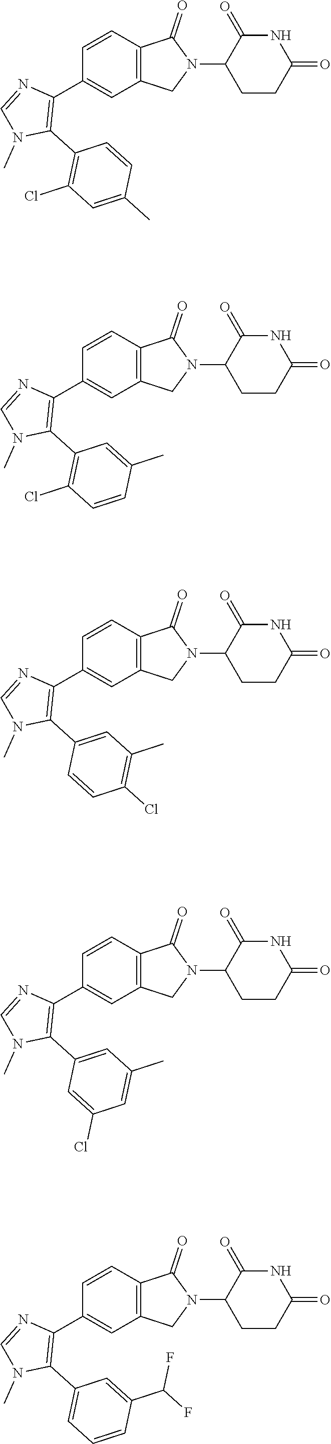 [SMILES: Cc1ccc(-c2c(-c3ccc4c(c3)CN(C3CCC(=O)NC3=O)C4=O)ncn2C)c(Cl)c1 ; Cc1ccc(Cl)c(-c2c(-c3ccc4c(c3)CN(C3CCC(=O)NC3=O)C4=O)ncn2C)c1 ; Cc1cc(-c2c(-c3ccc4c(c3)CN(C3CCC(=O)NC3=O)C4=O)ncn2C)ccc1Cl ; Cc1cc(Cl)cc(-c2c(-c3ccc4c(c3)CN(C3CCC(=O)NC3=O)C4=O)ncn2C)c1 ; Cn1cnc(-c2ccc3c(c2)CN(C2CCC(=O)NC2=O)C3=O)c1-c1cccc(C(F)F)c1]

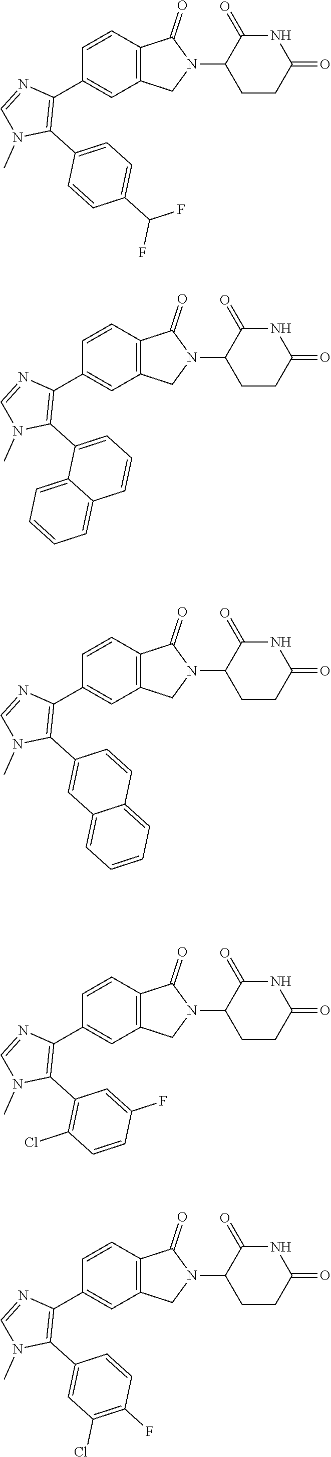 [SMILES: Cn1cnc(-c2ccc3c(c2)CN(C2CCC(=O)NC2=O)C3=O)c1-c1ccc(C(F)F)cc1 ; Cn1cnc(-c2ccc3c(c2)CN(C2CCC(=O)NC2=O)C3=O)c1-c1cccc2ccccc12 ; Cn1cnc(-c2ccc3c(c2)CN(C2CCC(=O)NC2=O)C3=O)c1-c1ccc2ccccc2c1 ; Cn1cnc(-c2ccc3c(c2)CN(C2CCC(=O)NC2=O)C3=O)c1-c1cc(F)ccc1Cl ; Cn1cnc(-c2ccc3c(c2)CN(C2CCC(=O)NC2=O)C3=O)c1-c1ccc(F)c(Cl)c1]

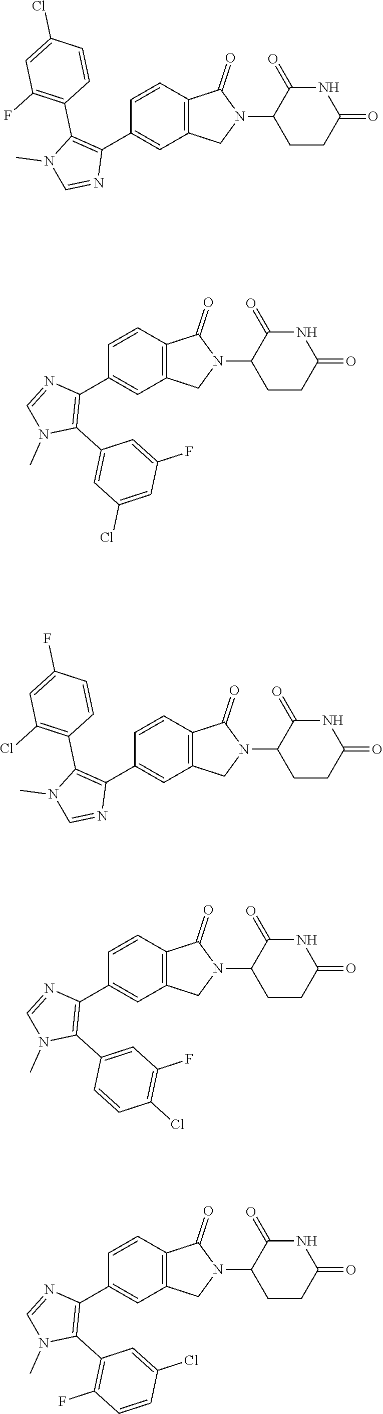 [SMILES: Cn1cnc(-c2ccc3c(c2)CN(C2CCC(=O)NC2=O)C3=O)c1-c1ccc(Cl)cc1F ; Cn1cnc(-c2ccc3c(c2)CN(C2CCC(=O)NC2=O)C3=O)c1-c1cc(F)cc(Cl)c1 ; Cn1cnc(-c2ccc3c(c2)CN(C2CCC(=O)NC2=O)C3=O)c1-c1ccc(F)cc1Cl ; Cn1cnc(-c2ccc3c(c2)CN(C2CCC(=O)NC2=O)C3=O)c1-c1ccc(Cl)c(F)c1 ; Cn1cnc(-c2ccc3c(c2)CN(C2CCC(=O)NC2=O)C3=O)c1-c1cc(Cl)ccc1F]

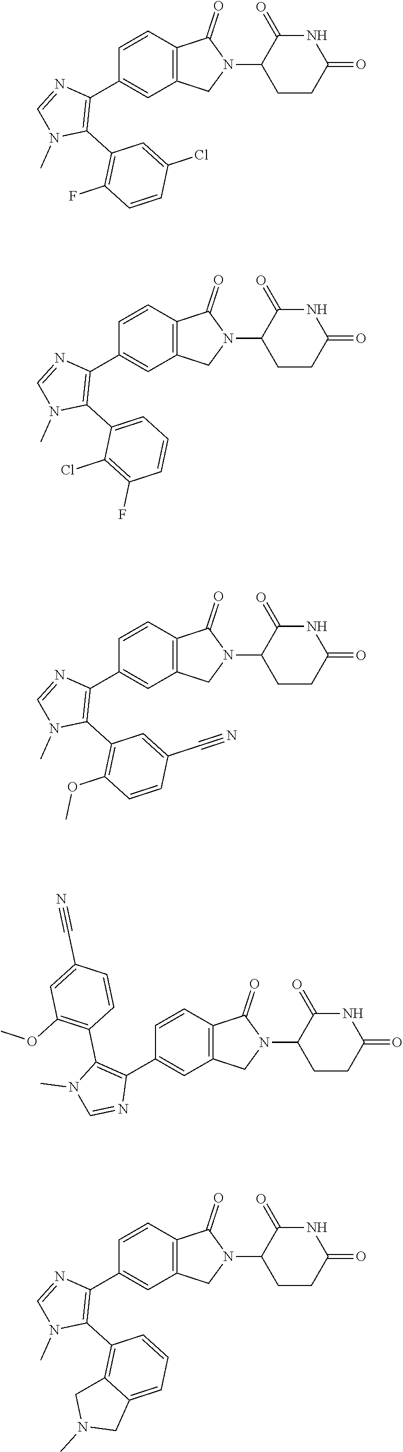 [SMILES: Cn1cnc(-c2ccc3c(c2)CN(C2CCC(=O)NC2=O)C3=O)c1-c1cc(Cl)ccc1F ; Cn1cnc(-c2ccc3c(c2)CN(C2CCC(=O)NC2=O)C3=O)c1-c1cccc(F)c1Cl ; COc1ccc(C#N)cc1-c1c(-c2ccc3c(c2)CN(C2CCC(=O)NC2=O)C3=O)ncn1C ; COc1cc(C#N)ccc1-c1c(-c2ccc3c(c2)CN(C2CCC(=O)NC2=O)C3=O)ncn1C ; CN1Cc2cccc(-c3c(-c4ccc5c(c4)CN(C4CCC(=O)NC4=O)C5=O)ncn3C)c2C1]

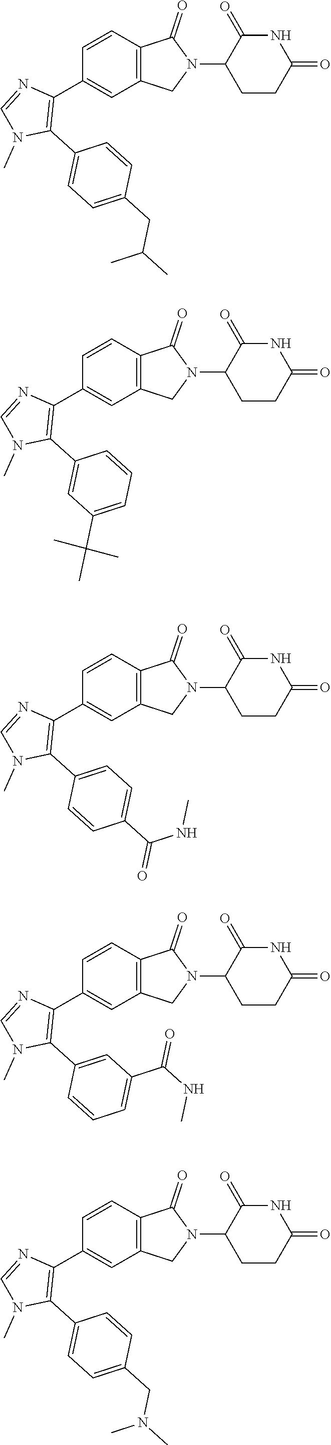

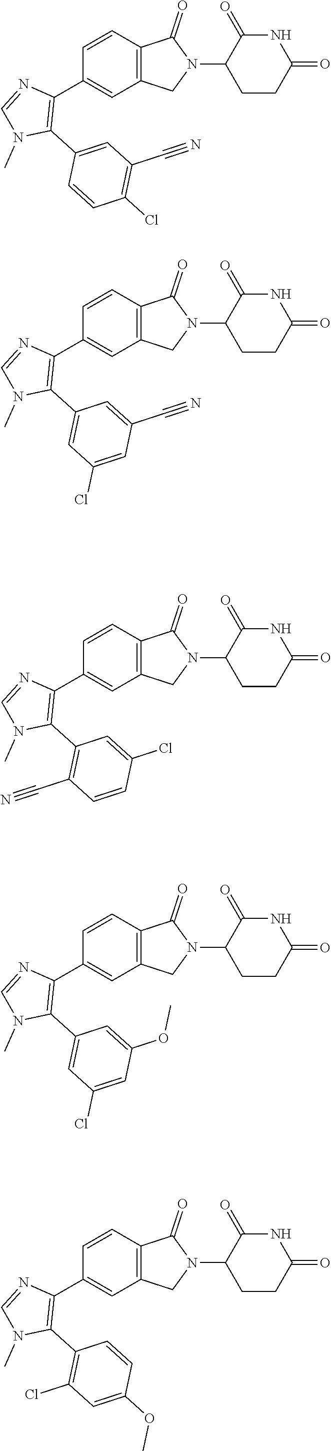 [SMILES: Cn1cnc(-c2ccc3c(c2)CN(C2CCC(=O)NC2=O)C3=O)c1-c1ccc(Cl)c(C#N)c1 ; Cn1cnc(-c2ccc3c(c2)CN(C2CCC(=O)NC2=O)C3=O)c1-c1cc(Cl)cc(C#N)c1 ; Cn1cnc(-c2ccc3c(c2)CN(C2CCC(=O)NC2=O)C3=O)c1-c1cc(Cl)ccc1C#N ; COc1cc(Cl)cc(-c2c(-c3ccc4c(c3)CN(C3CCC(=O)NC3=O)C4=O)ncn2C)c1 ; COc1ccc(-c2c(-c3ccc4c(c3)CN(C3CCC(=O)NC3=O)C4=O)ncn2C)c(Cl)c1]

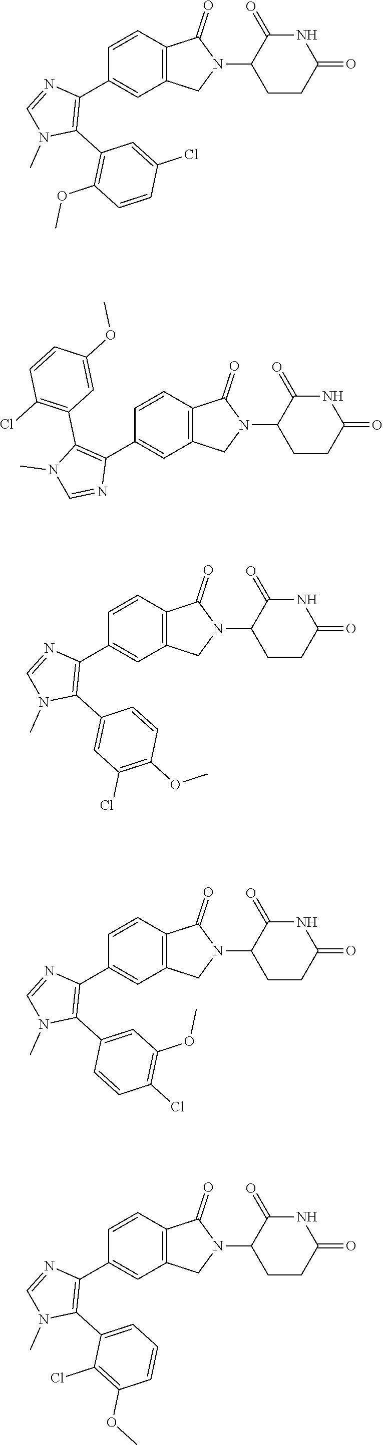 [SMILES: COc1ccc(Cl)cc1-c1c(-c2ccc3c(c2)CN(C2CCC(=O)NC2=O)C3=O)ncn1C ; COc1ccc(Cl)c(-c2c(-c3ccc4c(c3)CN(C3CCC(=O)NC3=O)C4=O)ncn2C)c1 ; COc1ccc(-c2c(-c3ccc4c(c3)CN(C3CCC(=O)NC3=O)C4=O)ncn2C)cc1Cl ; COc1cc(-c2c(-c3ccc4c(c3)CN(C3CCC(=O)NC3=O)C4=O)ncn2C)ccc1Cl ; COc1cccc(-c2c(-c3ccc4c(c3)CN(C3CCC(=O)NC3=O)C4=O)ncn2C)c1Cl]

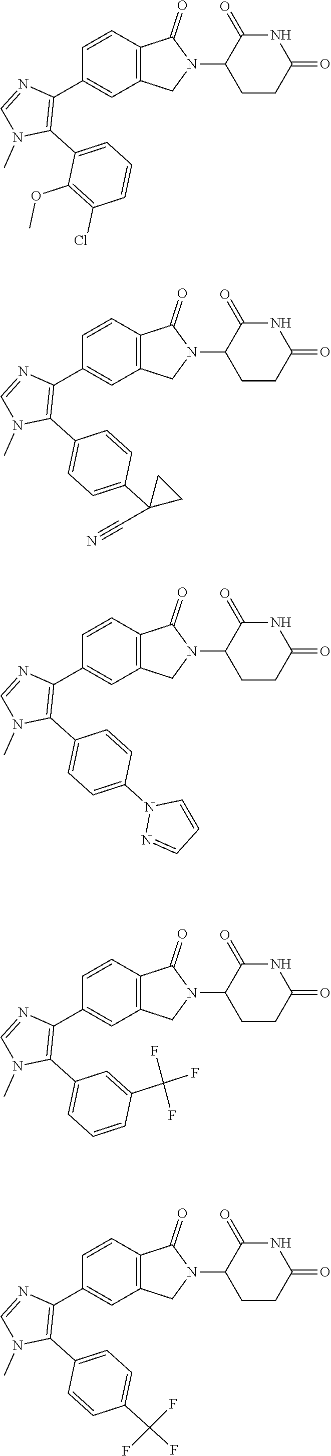 [SMILES: COc1c(Cl)cccc1-c1c(-c2ccc3c(c2)CN(C2CCC(=O)NC2=O)C3=O)ncn1C ; Cn1cnc(-c2ccc3c(c2)CN(C2CCC(=O)NC2=O)C3=O)c1-c1ccc(C2(C#N)CC2)cc1 ; Cn1cnc(-c2ccc3c(c2)CN(C2CCC(=O)NC2=O)C3=O)c1-c1ccc(-n2cccn2)cc1 ; Cn1cnc(-c2ccc3c(c2)CN(C2CCC(=O)NC2=O)C3=O)c1-c1cccc(C(F)(F)F)c1 ; Cn1cnc(-c2ccc3c(c2)CN(C2CCC(=O)NC2=O)C3=O)c1-c1ccc(C(F)(F)F)cc1]

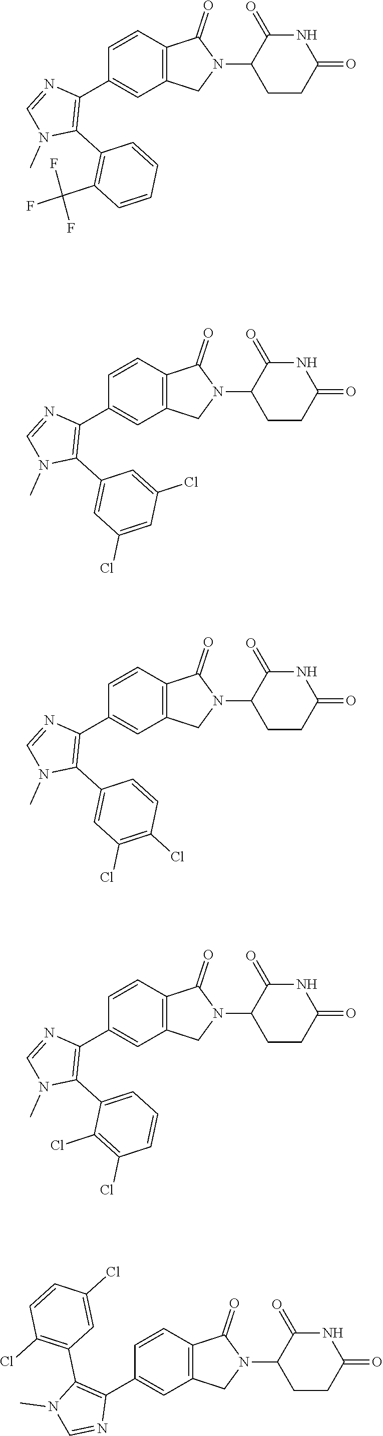 [SMILES: Cn1cnc(-c2ccc3c(c2)CN(C2CCC(=O)NC2=O)C3=O)c1-c1ccccc1C(F)(F)F ; Cn1cnc(-c2ccc3c(c2)CN(C2CCC(=O)NC2=O)C3=O)c1-c1cc(Cl)cc(Cl)c1 ; Cn1cnc(-c2ccc3c(c2)CN(C2CCC(=O)NC2=O)C3=O)c1-c1ccc(Cl)c(Cl)c1 ; Cn1cnc(-c2ccc3c(c2)CN(C2CCC(=O)NC2=O)C3=O)c1-c1cccc(Cl)c1Cl ; Cn1cnc(-c2ccc3c(c2)CN(C2CCC(=O)NC2=O)C3=O)c1-c1cc(Cl)ccc1Cl]

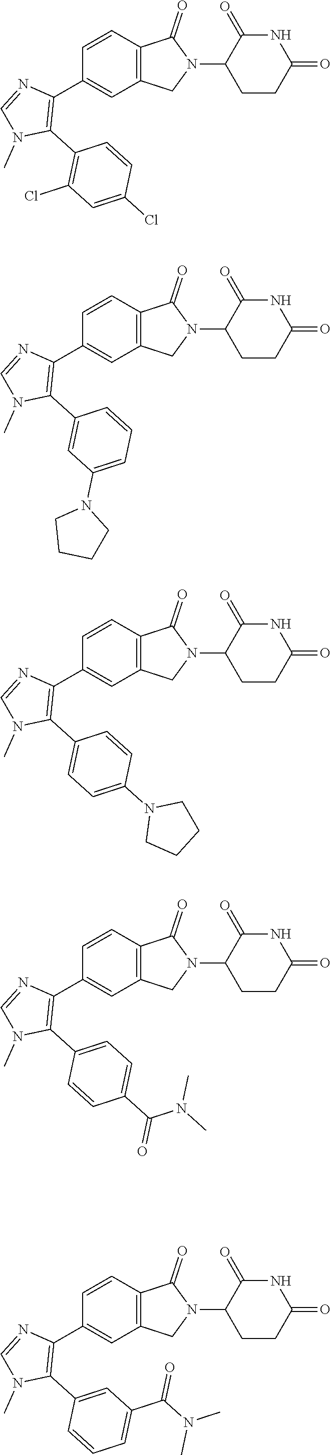 [SMILES: Cn1cnc(-c2ccc3c(c2)CN(C2CCC(=O)NC2=O)C3=O)c1-c1ccc(Cl)cc1Cl ; Cn1cnc(-c2ccc3c(c2)CN(C2CCC(=O)NC2=O)C3=O)c1-c1cccc(N2CCCC2)c1 ; Cn1cnc(-c2ccc3c(c2)CN(C2CCC(=O)NC2=O)C3=O)c1-c1ccc(N2CCCC2)cc1 ; CN(C)C(=O)c1ccc(-c2c(-c3ccc4c(c3)CN(C3CCC(=O)NC3=O)C4=O)ncn2C)cc1 ; CN(C)C(=O)c1cccc(-c2c(-c3ccc4c(c3)CN(C3CCC(=O)NC3=O)C4=O)ncn2C)c1]

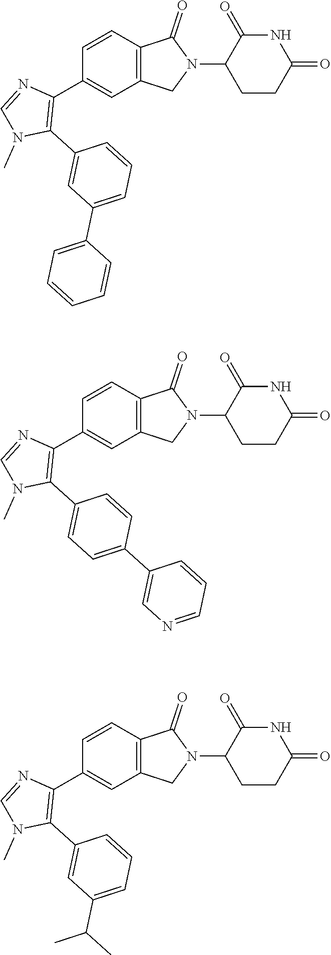 [SMILES: Cn1cnc(-c2ccc3c(c2)CN(C2CCC(=O)NC2=O)C3=O)c1-c1cccc(-c2ccccc2)c1 ; Cn1cnc(-c2ccc3c(c2)CN(C2CCC(=O)NC2=O)C3=O)c1-c1ccc(-c2cccnc2)cc1 ; CC(C)c1cccc(-c2c(-c3ccc4c(c3)CN(C3CCC(=O)NC3=O)C4=O)ncn2C)c1]

In another embodiment, the compound provided herein for use in the compositions and methods provided herein is selected from:

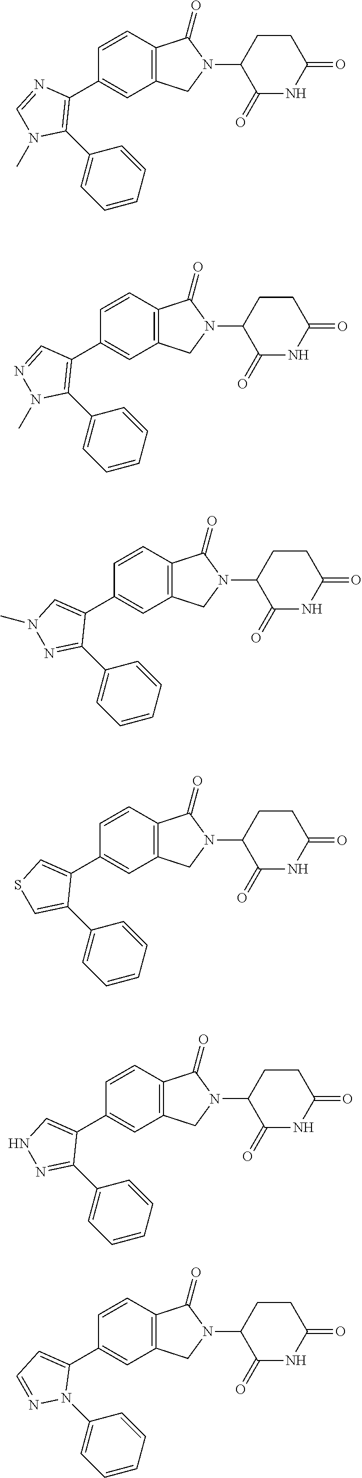 [SMILES: Cn1cnc(-c2ccc3c(c2)CN(C2CCC(=O)NC2=O)C3=O)c1-c1ccccc1 ; Cn1ncc(-c2ccc3c(c2)CN(C2CCC(=O)NC2=O)C3=O)c1-c1ccccc1 ; Cn1cc(-c2ccc3c(c2)CN(C2CCC(=O)NC2=O)C3=O)c(-c2ccccc2)n1 ; O=C1CCC(N2Cc3cc(-c4cscc4-c4ccccc4)ccc3C2=O)C(=O)N1 ; O=C1CCC(N2Cc3cc(-c4c[nH]nc4-c4ccccc4)ccc3C2=O)C(=O)N1 ; O=C1CCC(N2Cc3cc(-c4ccnn4-c4ccccc4)ccc3C2=O)C(=O)N1]

In another embodiment, the compound provided herein for use in the compositions and methods provided herein is selected from:

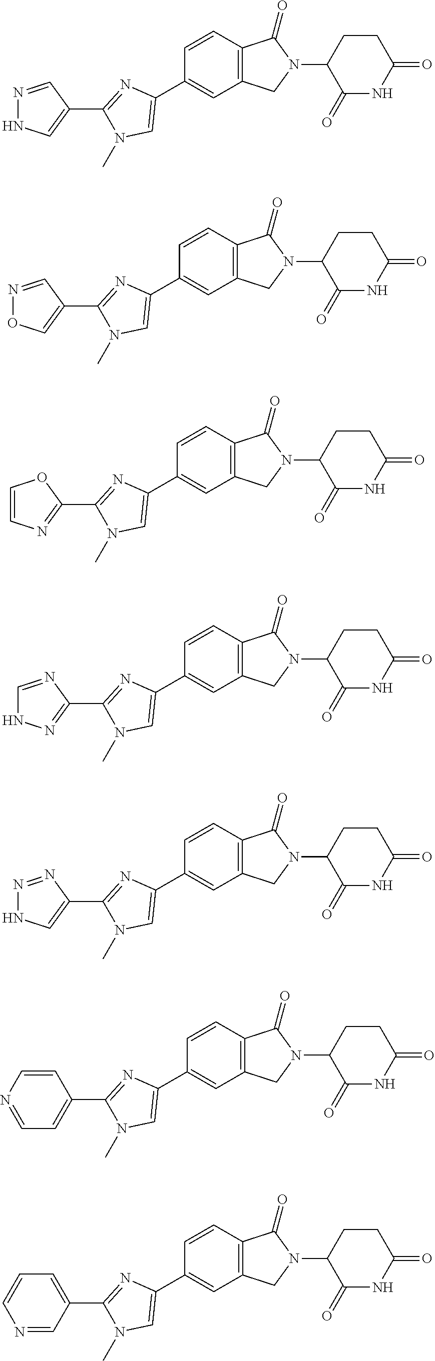

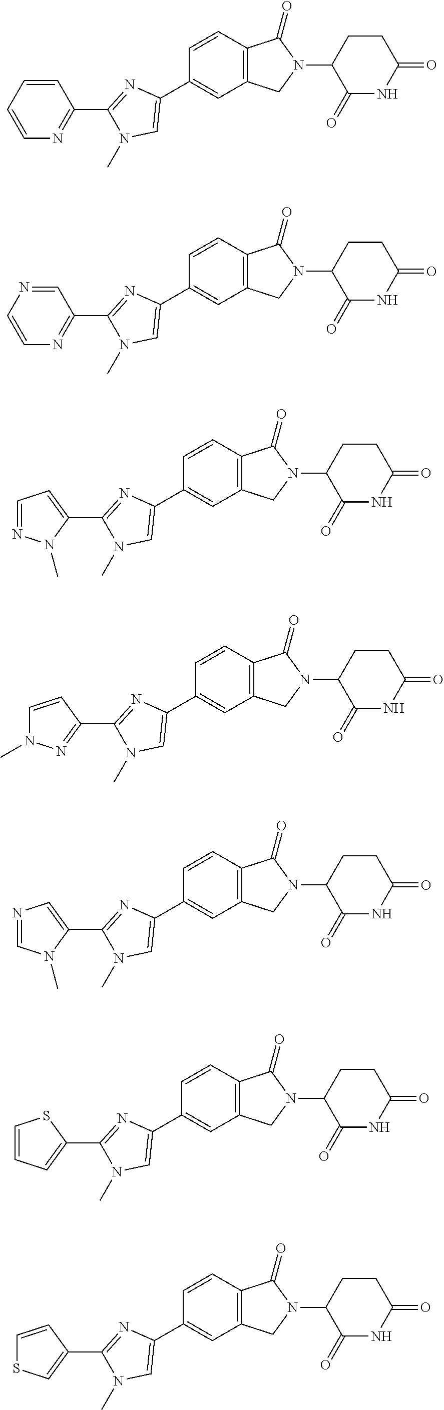

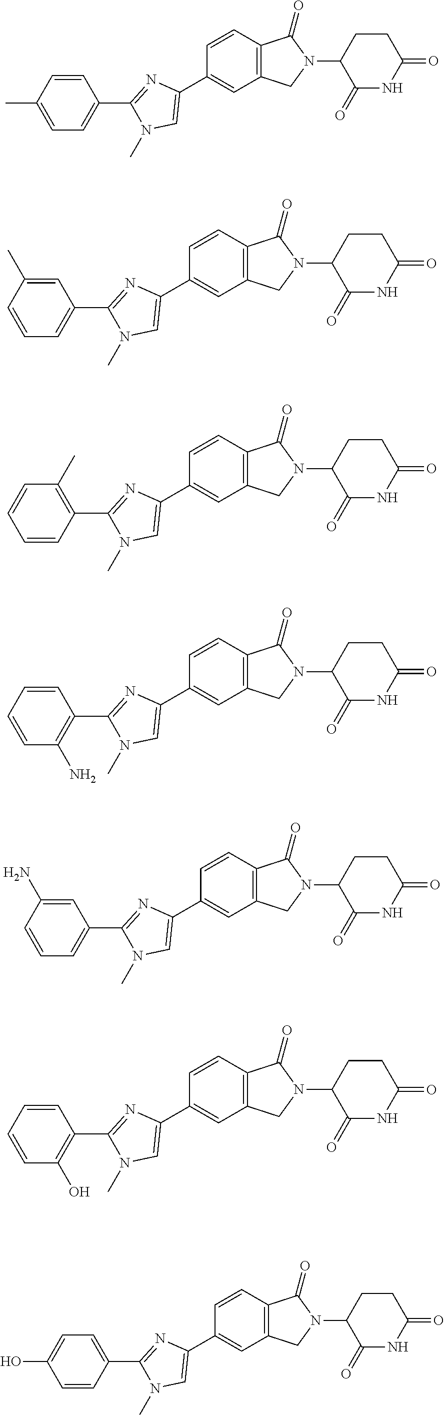

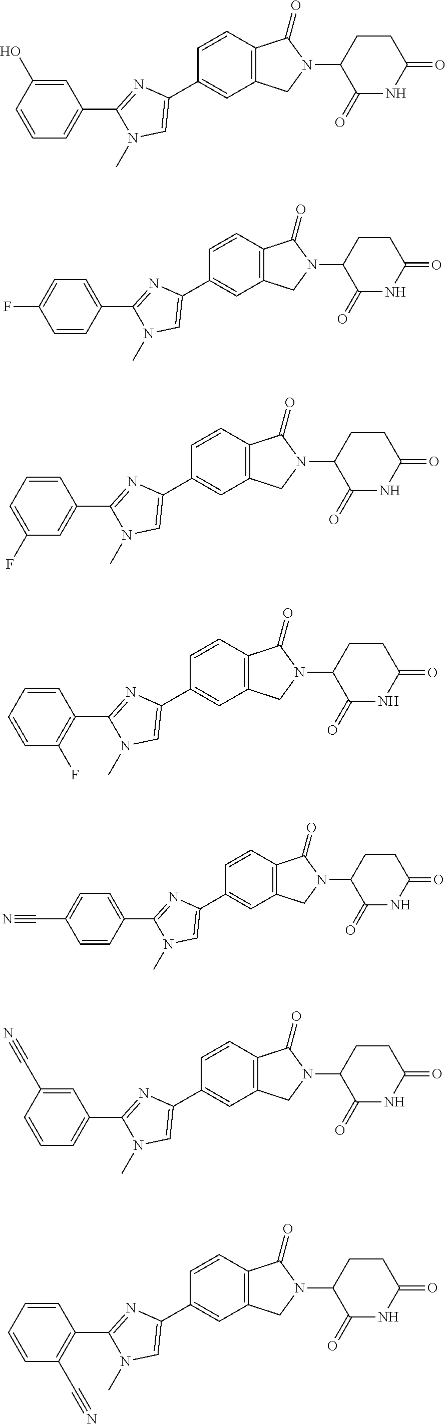

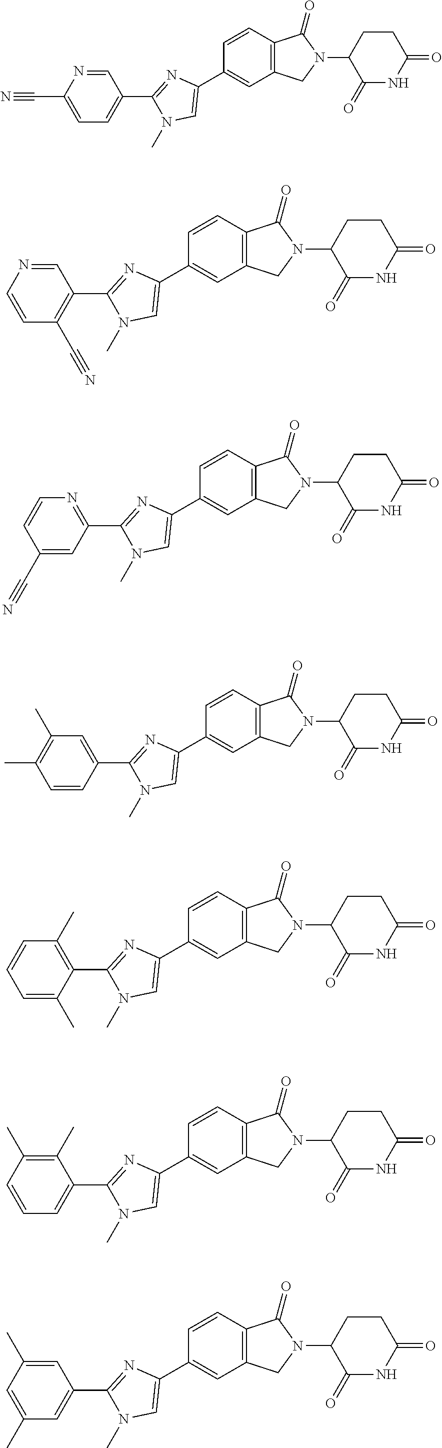

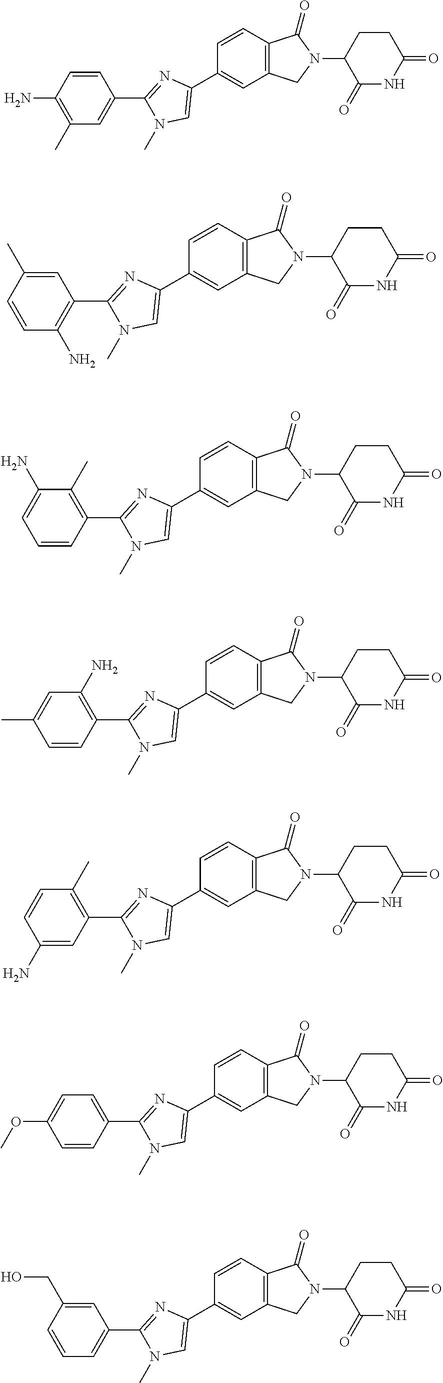

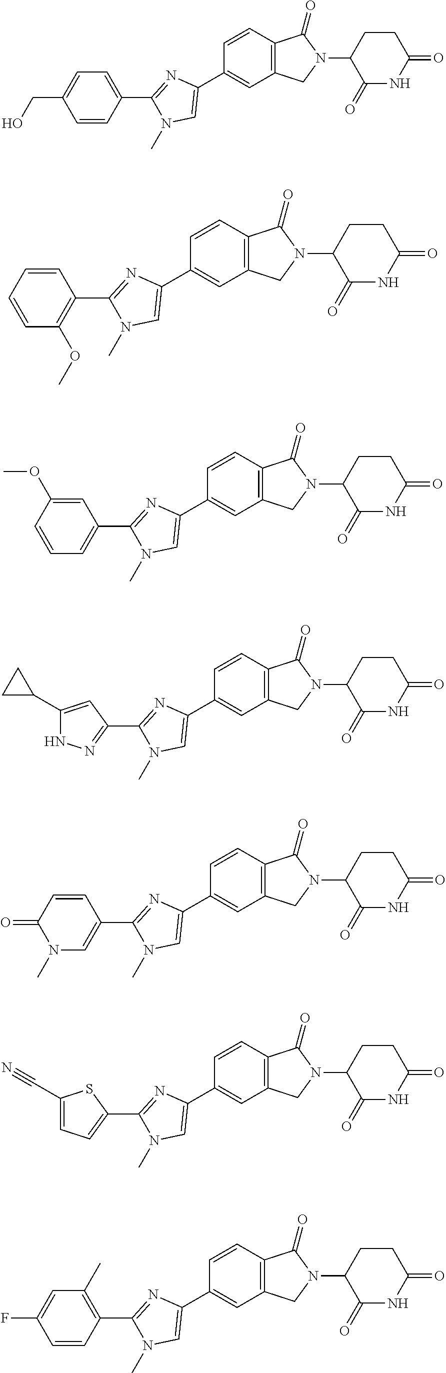

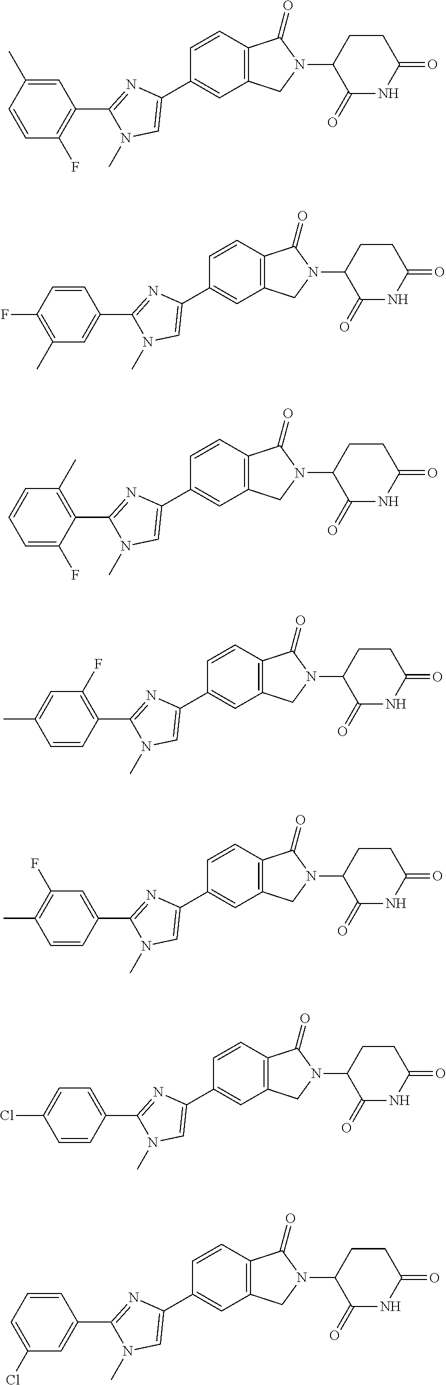

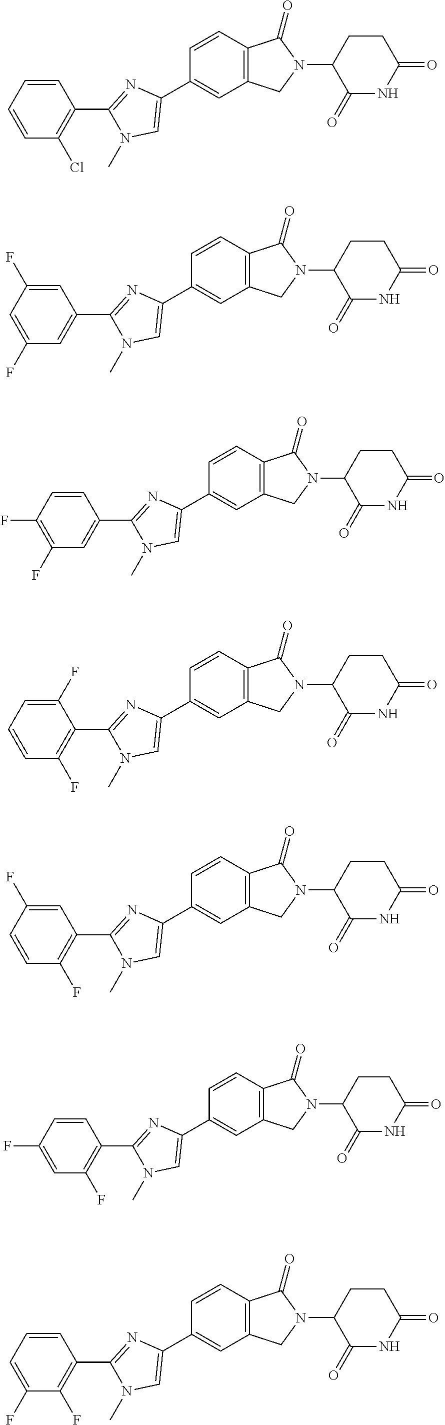

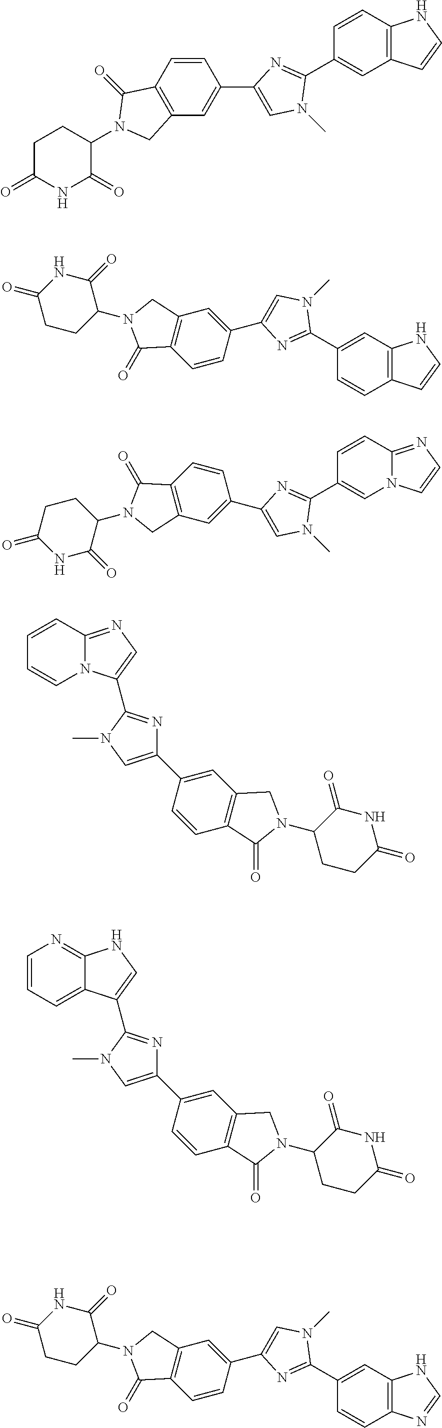

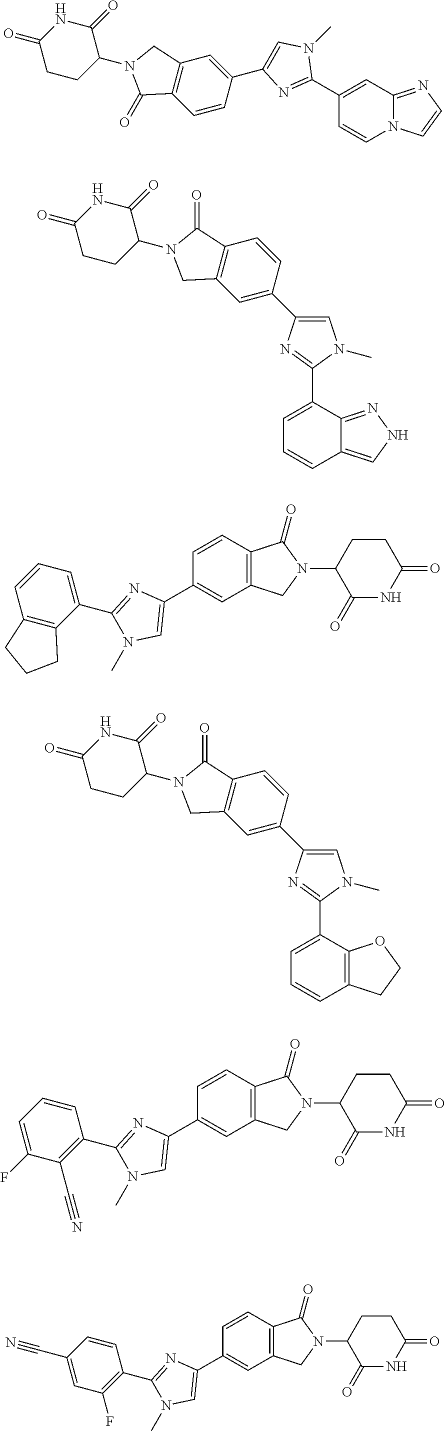

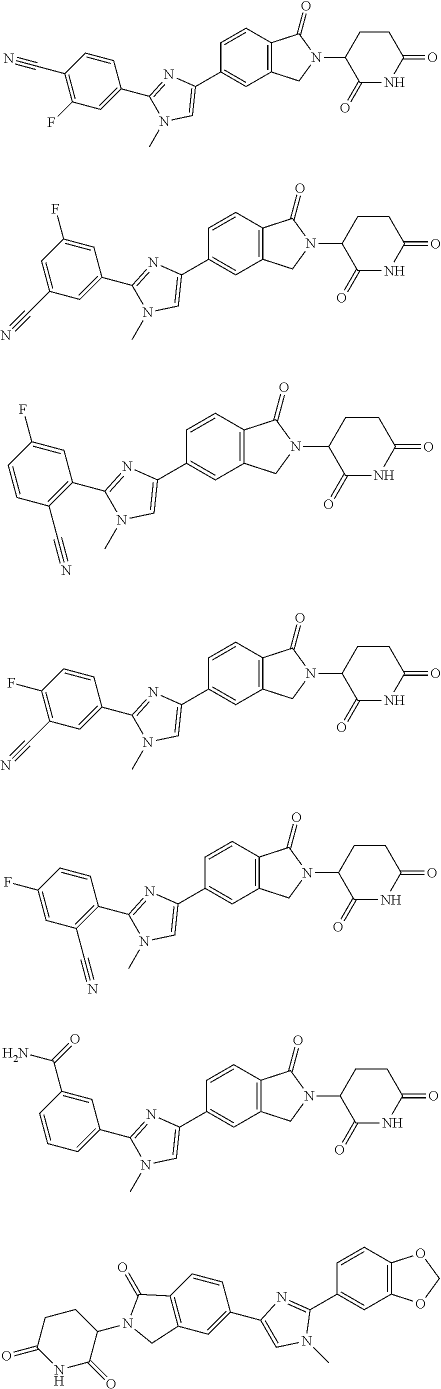

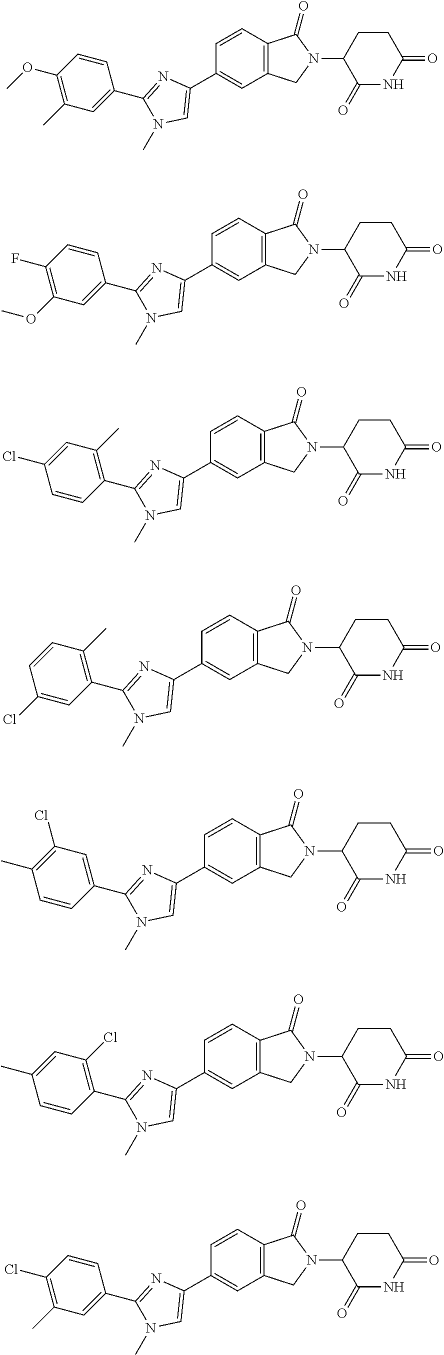

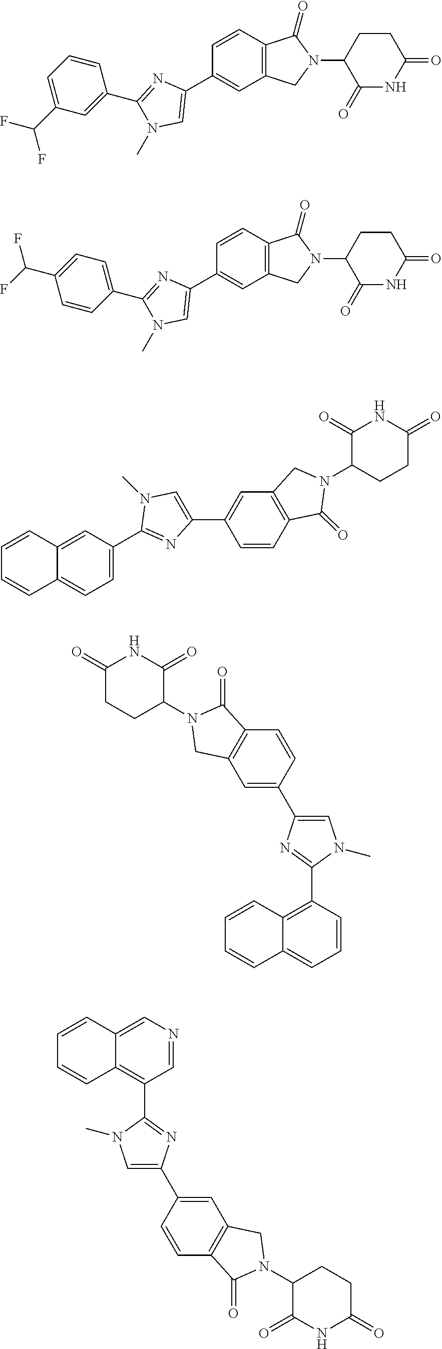 [SMILES: Cn1cc(-c2ccc3c(c2)CN(C2CCC(=O)NC2=O)C3=O)nc1-c1cccc(C(F)F)c1 ; Cn1cc(-c2ccc3c(c2)CN(C2CCC(=O)NC2=O)C3=O)nc1-c1ccc(C(F)F)cc1 ; Cn1cc(-c2ccc3c(c2)CN(C2CCC(=O)NC2=O)C3=O)nc1-c1ccc2ccccc2c1 ; Cn1cc(-c2ccc3c(c2)CN(C2CCC(=O)NC2=O)C3=O)nc1-c1cccc2ccccc12 ; Cn1cc(-c2ccc3c(c2)CN(C2CCC(=O)NC2=O)C3=O)nc1-c1cncc2ccccc12]

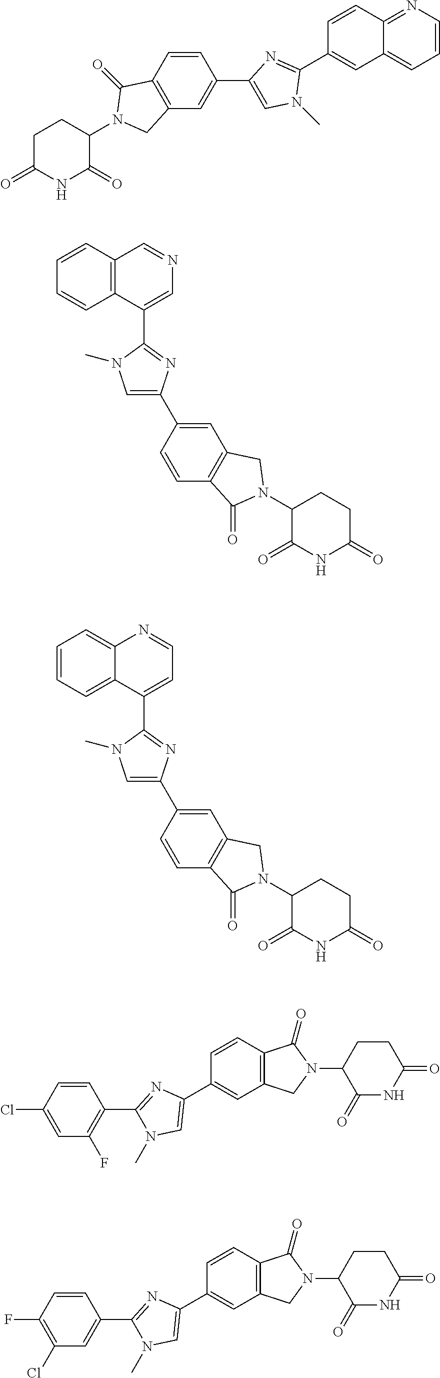

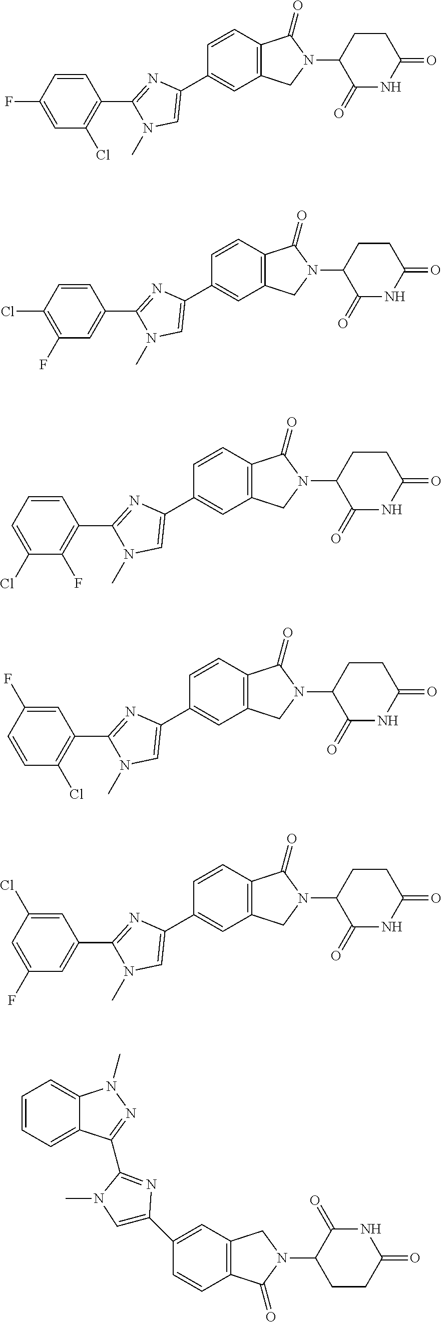 [SMILES: Cn1cc(-c2ccc3c(c2)CN(C2CCC(=O)NC2=O)C3=O)nc1-c1ccc(F)cc1Cl ; Cn1cc(-c2ccc3c(c2)CN(C2CCC(=O)NC2=O)C3=O)nc1-c1ccc(Cl)c(F)c1 ; Cn1cc(-c2ccc3c(c2)CN(C2CCC(=O)NC2=O)C3=O)nc1-c1cccc(Cl)c1F ; Cn1cc(-c2ccc3c(c2)CN(C2CCC(=O)NC2=O)C3=O)nc1-c1cc(F)ccc1Cl ; Cn1cc(-c2ccc3c(c2)CN(C2CCC(=O)NC2=O)C3=O)nc1-c1cc(F)cc(Cl)c1 ; Cn1cc(-c2ccc3c(c2)CN(C2CCC(=O)NC2=O)C3=O)nc1-c1nn(C)c2ccccc12]

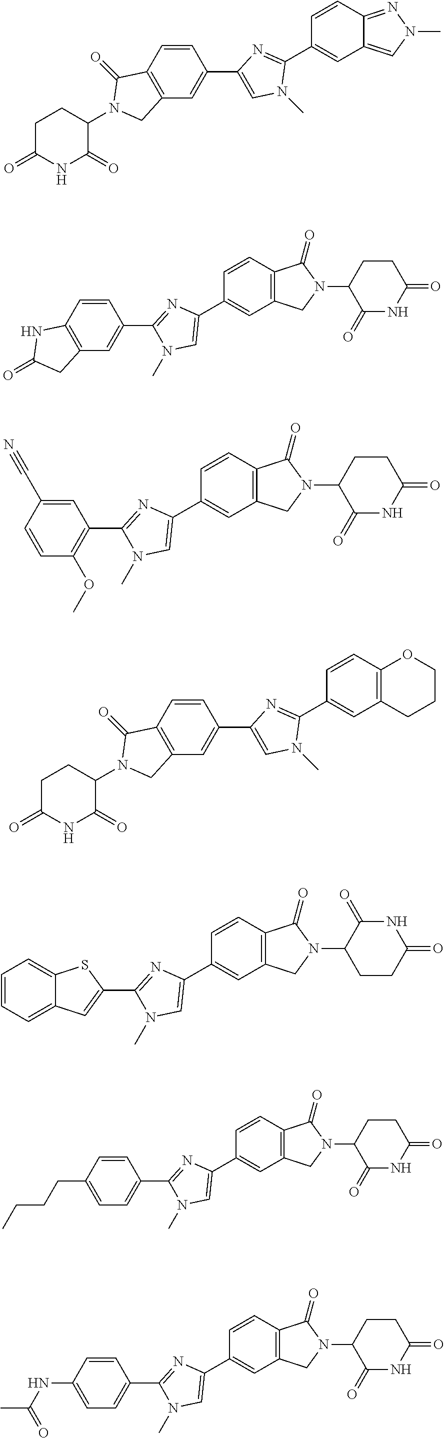

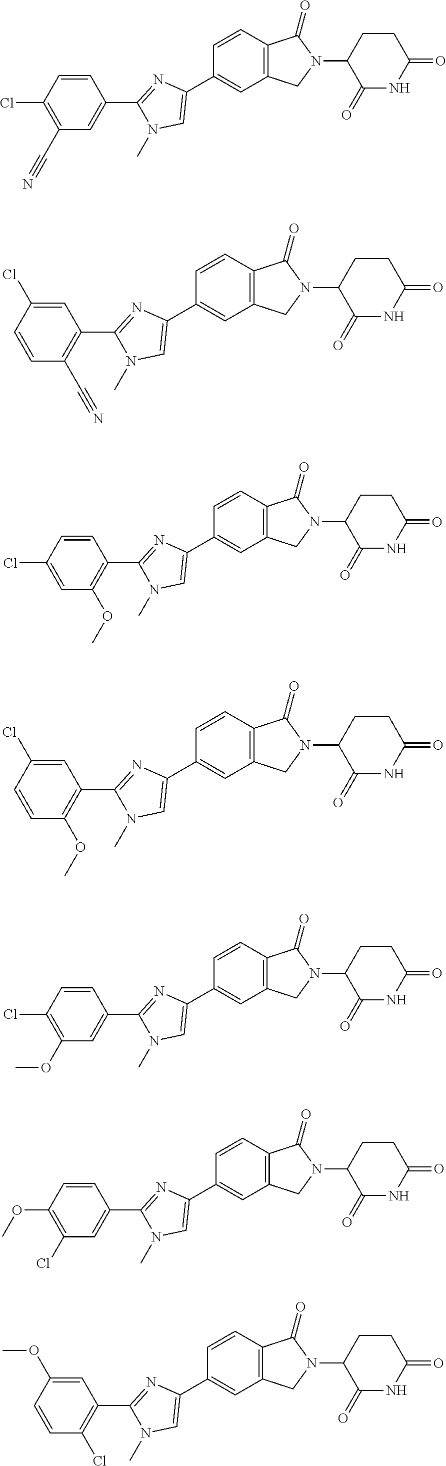

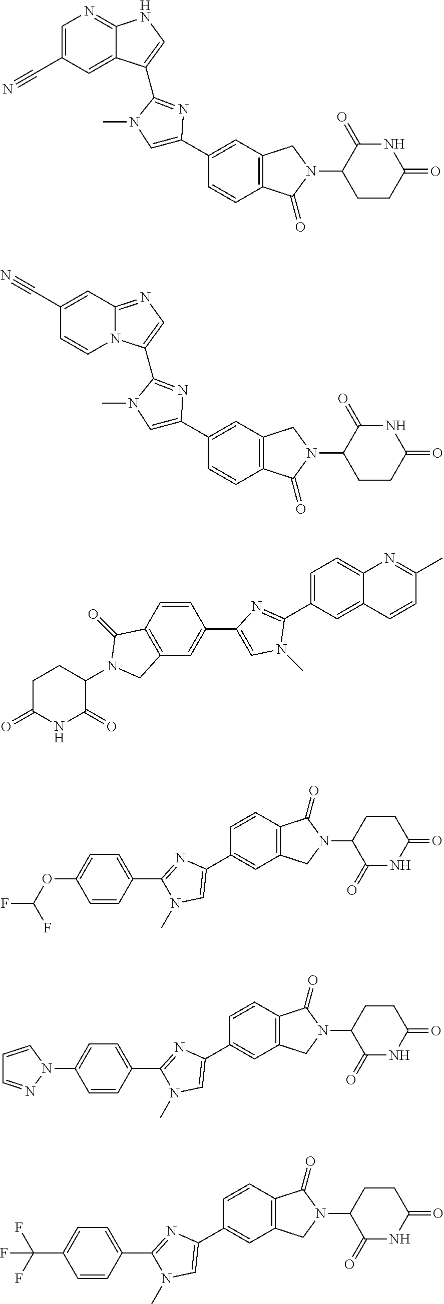 [SMILES: Cn1cc(-c2ccc3c(c2)CN(C2CCC(=O)NC2=O)C3=O)nc1-c1c[nH]c2ncc(C#N)cc12 ; Cn1cc(-c2ccc3c(c2)CN(C2CCC(=O)NC2=O)C3=O)nc1-c1cnc2cc(C#N)ccn12 ; Cc1ccc2cc(-c3nc(-c4ccc5c(c4)CN(C4CCC(=O)NC4=O)C5=O)cn3C)ccc2n1 ; Cn1cc(-c2ccc3c(c2)CN(C2CCC(=O)NC2=O)C3=O)nc1-c1ccc(OC(F)F)cc1 ; Cn1cc(-c2ccc3c(c2)CN(C2CCC(=O)NC2=O)C3=O)nc1-c1ccc(-n2cccn2)cc1 ; Cn1cc(-c2ccc3c(c2)CN(C2CCC(=O)NC2=O)C3=O)nc1-c1ccc(C(F)(F)F)cc1]

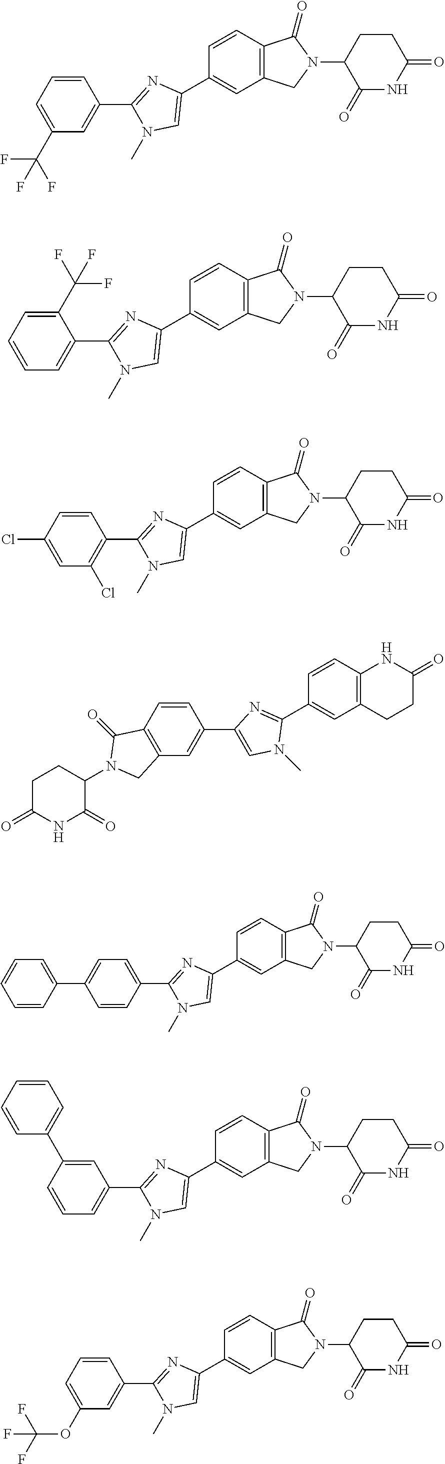

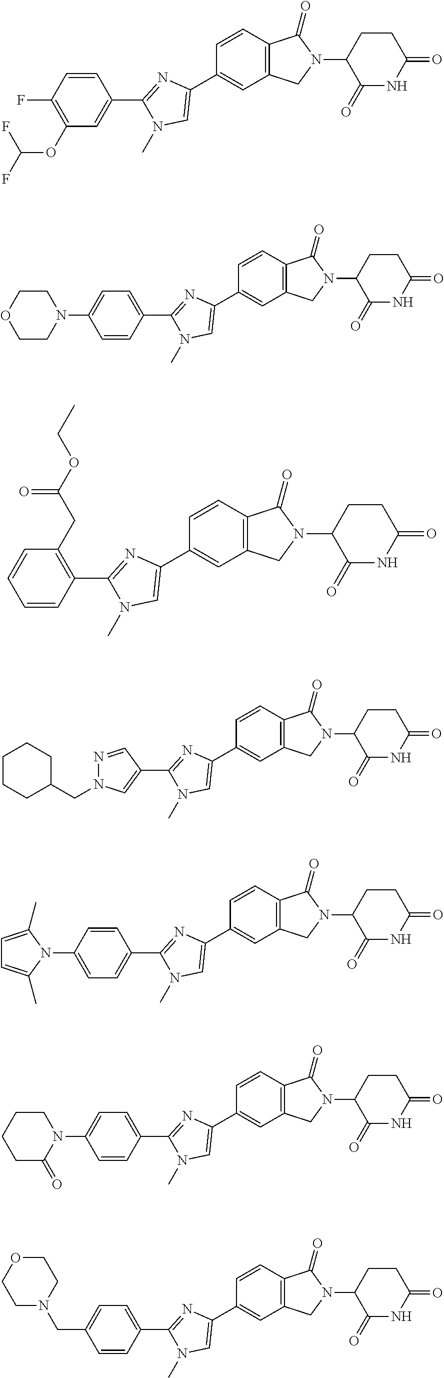

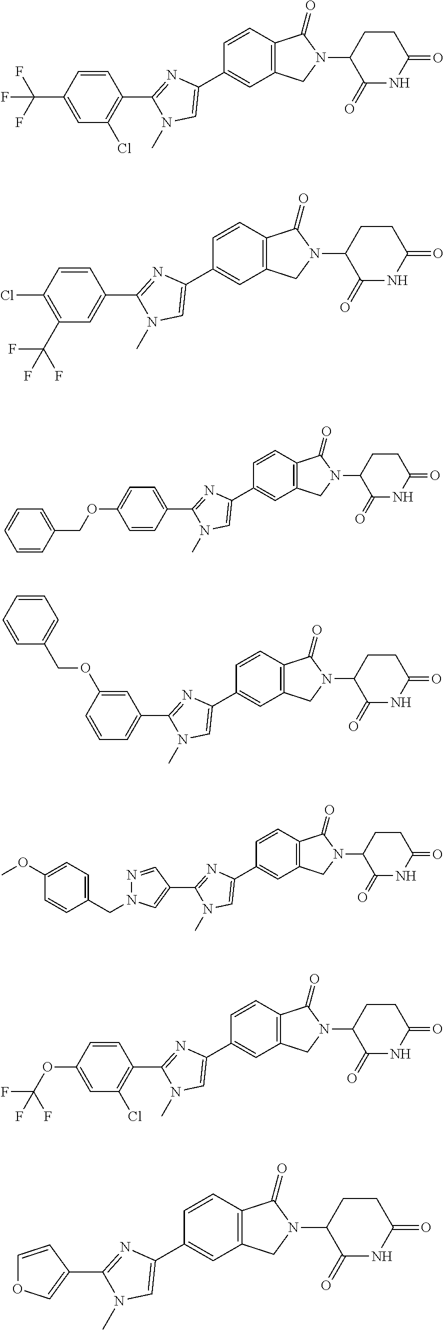

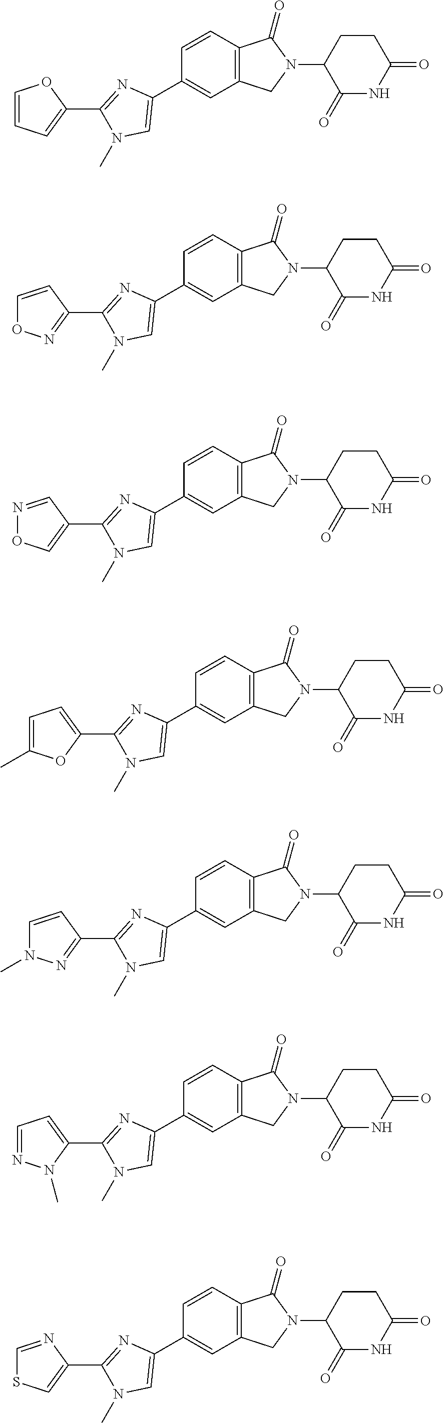

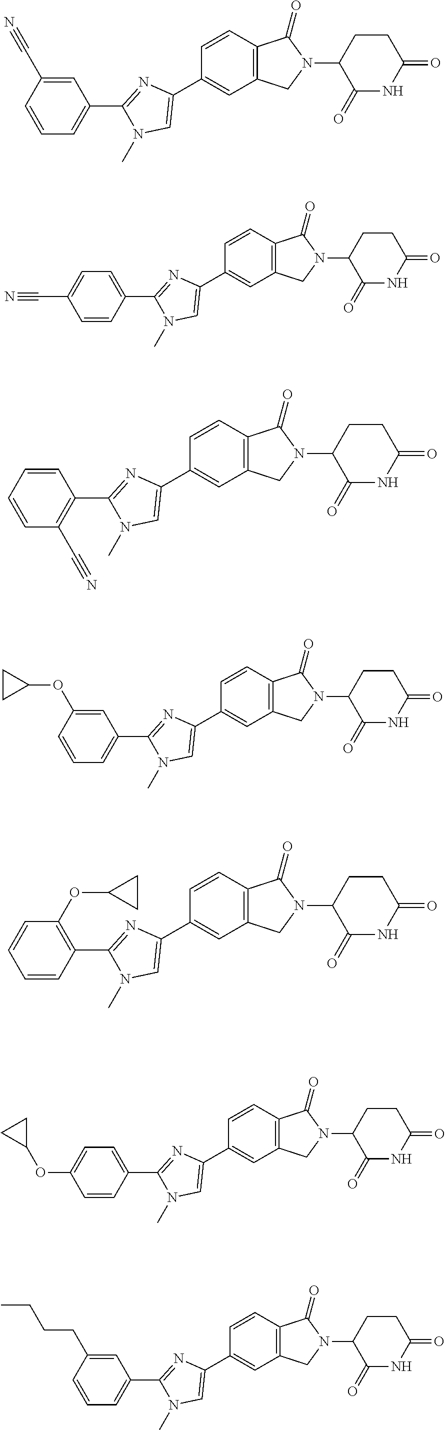

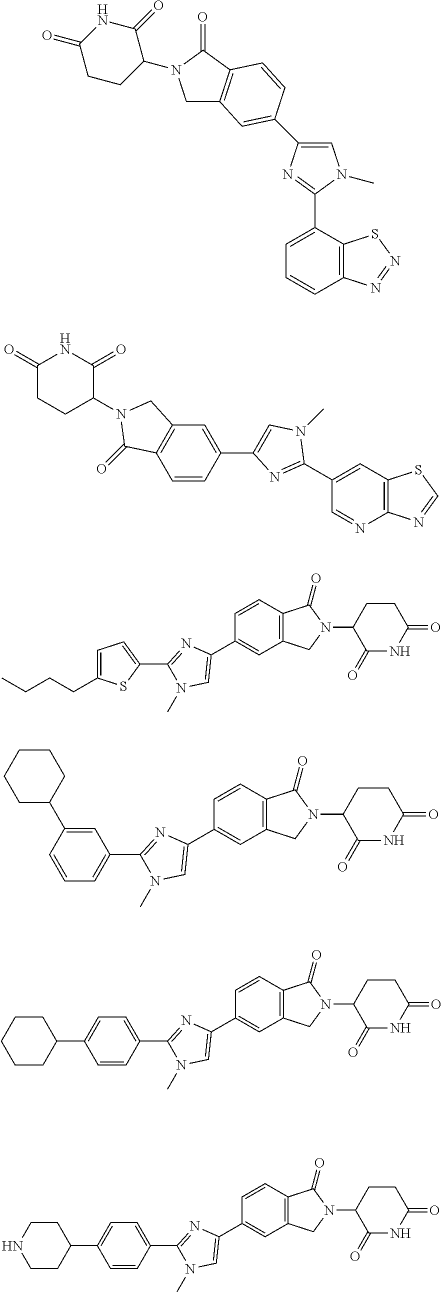 [SMILES: Cn1cc(-c2ccc3c(c2)CN(C2CCC(=O)NC2=O)C3=O)nc1-c1cccc2nnsc12 ; Cn1cc(-c2ccc3c(c2)CN(C2CCC(=O)NC2=O)C3=O)nc1-c1cnc2ncsc2c1 ; CCCCc1ccc(-c2nc(-c3ccc4c(c3)CN(C3CCC(=O)NC3=O)C4=O)cn2C)s1 ; Cn1cc(-c2ccc3c(c2)CN(C2CCC(=O)NC2=O)C3=O)nc1-c1cccc(C2CCCCC2)c1 ; Cn1cc(-c2ccc3c(c2)CN(C2CCC(=O)NC2=O)C3=O)nc1-c1ccc(C2CCCCC2)cc1 ; Cn1cc(-c2ccc3c(c2)CN(C2CCC(=O)NC2=O)C3=O)nc1-c1ccc(C2CCNCC2)cc1]

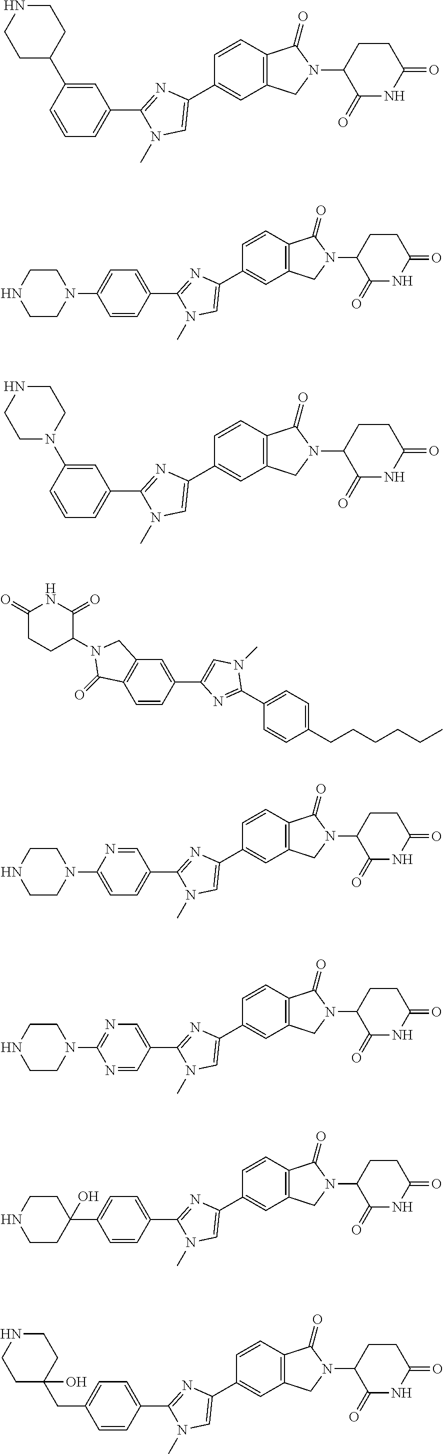

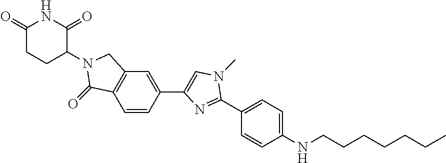 [SMILES: CCCCCCCNc1ccc(-c2nc(-c3ccc4c(c3)CN(C3CCC(=O)NC3=O)C4=O)cn2C)cc1]

In another embodiment, the compound provided herein for use in the compositions and methods provided herein is selected from:

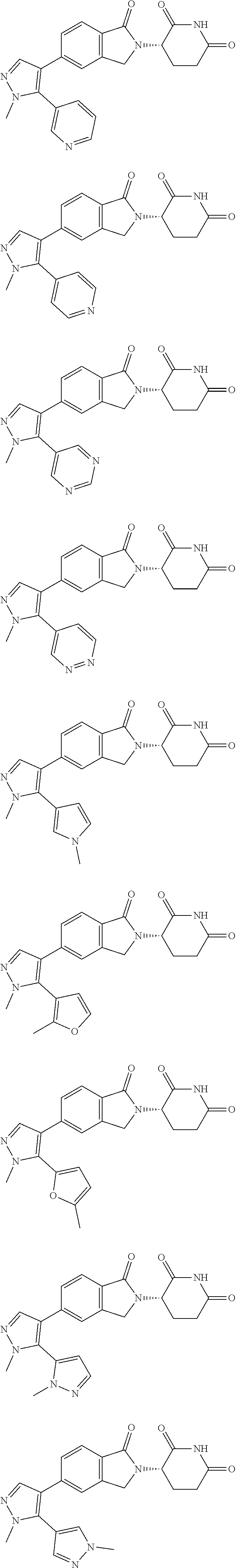

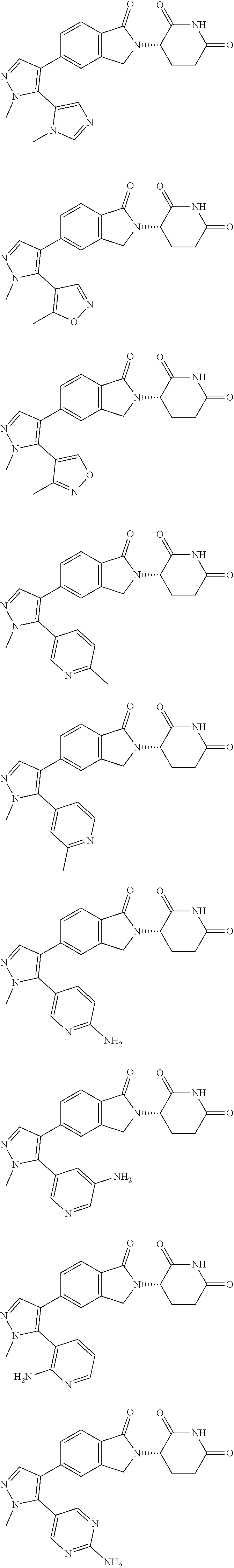

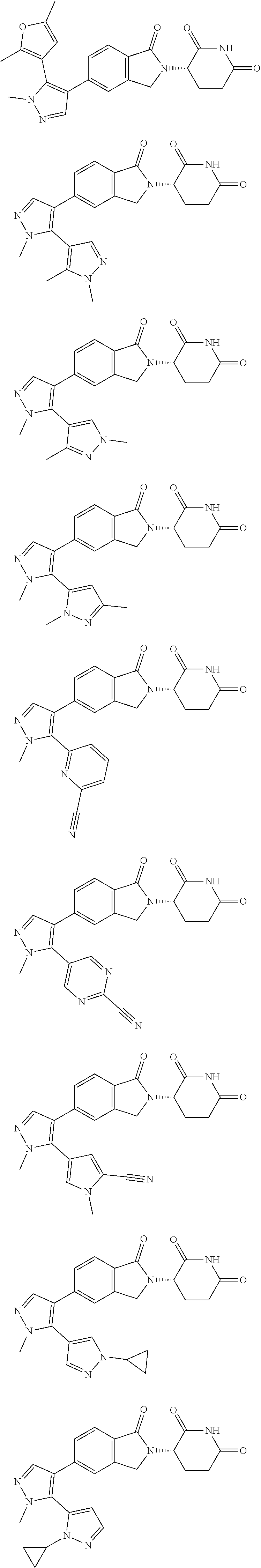

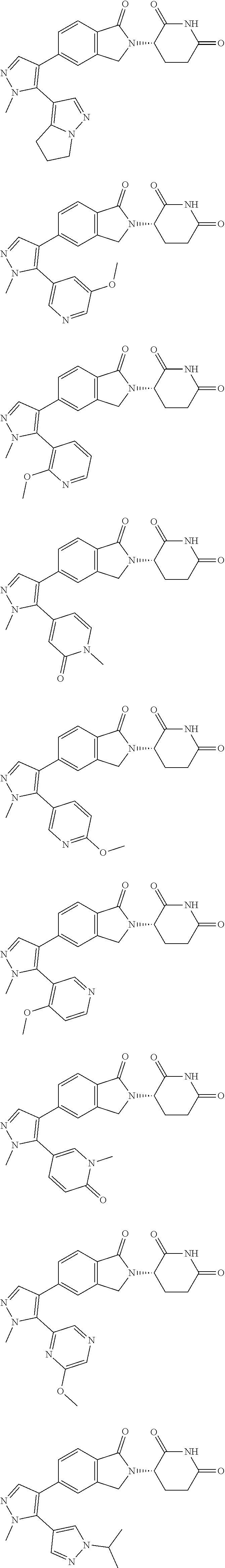

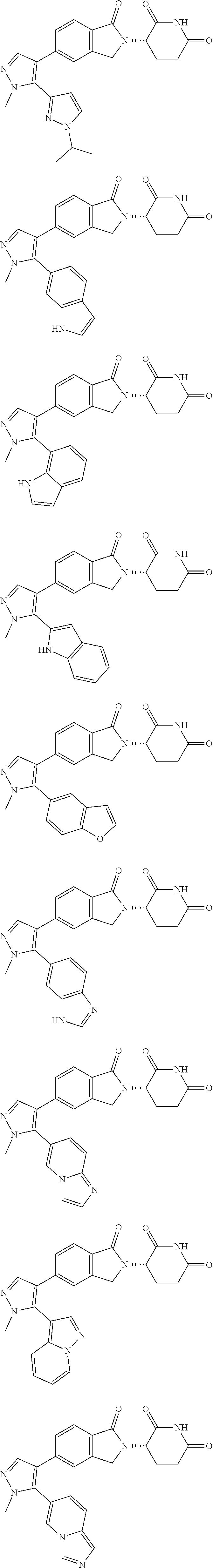

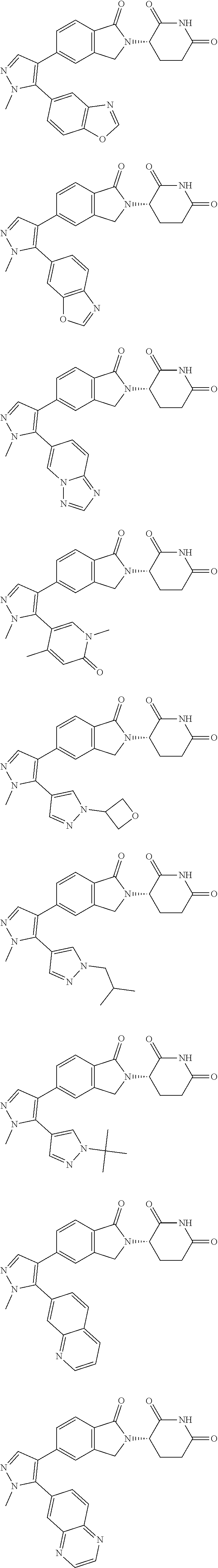

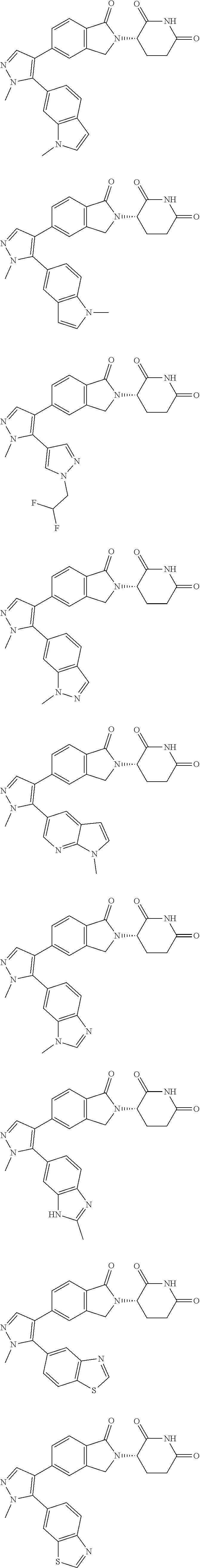

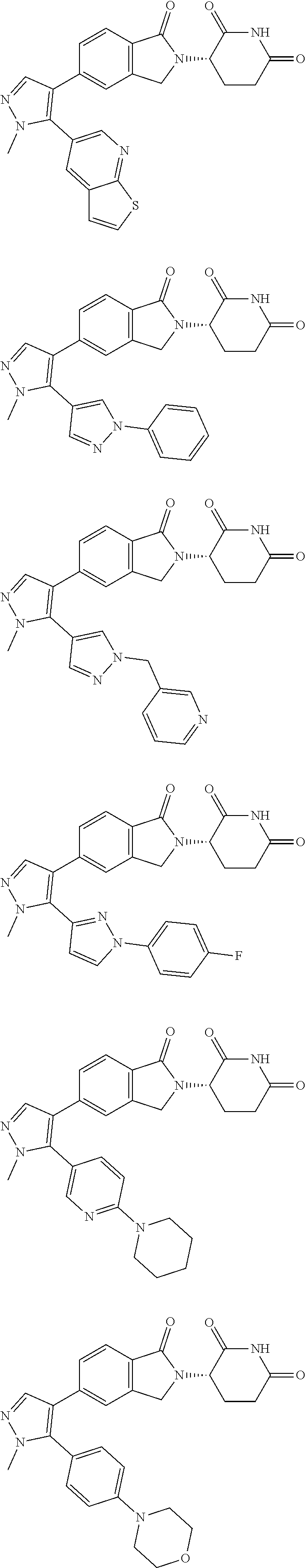 [SMILES: Cn1ncc(-c2ccc3c(c2)CN([C@H]2CCC(=O)NC2=O)C3=O)c1-c1cnc2sccc2c1 ; Cn1ncc(-c2ccc3c(c2)CN([C@H]2CCC(=O)NC2=O)C3=O)c1-c1cnn(-c2ccccc2)c1 ; Cn1ncc(-c2ccc3c(c2)CN([C@H]2CCC(=O)NC2=O)C3=O)c1-c1cnn(Cc2cccnc2)c1 ; Cn1ncc(-c2ccc3c(c2)CN([C@H]2CCC(=O)NC2=O)C3=O)c1-c1ccn(-c2ccc(F)cc2)n1 ; Cn1ncc(-c2ccc3c(c2)CN([C@H]2CCC(=O)NC2=O)C3=O)c1-c1ccc(N2CCCCC2)nc1 ; Cn1ncc(-c2ccc3c(c2)CN([C@H]2CCC(=O)NC2=O)C3=O)c1-c1ccc(N2CCOCC2)cc1]

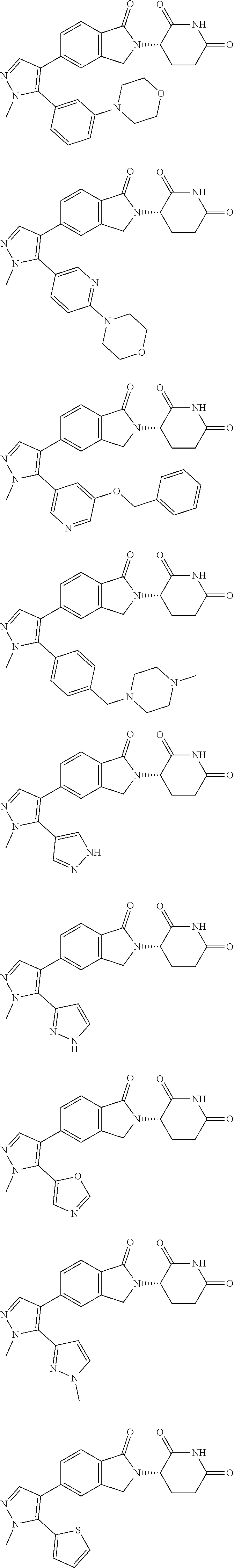

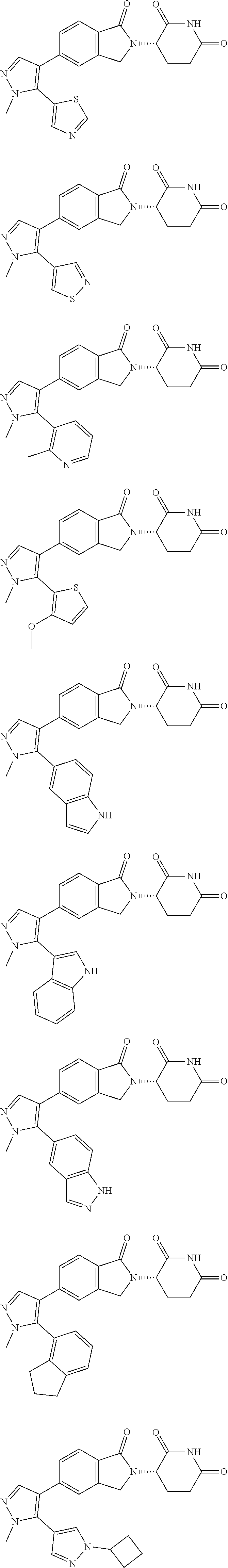

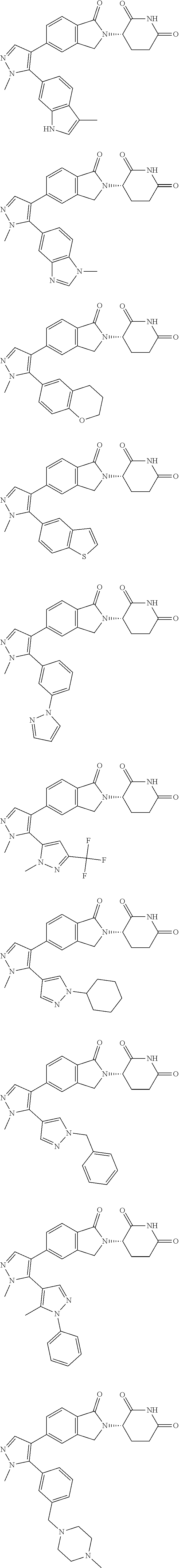

In another embodiment, the compound provided herein for use in the compositions and methods provided herein is selected from:

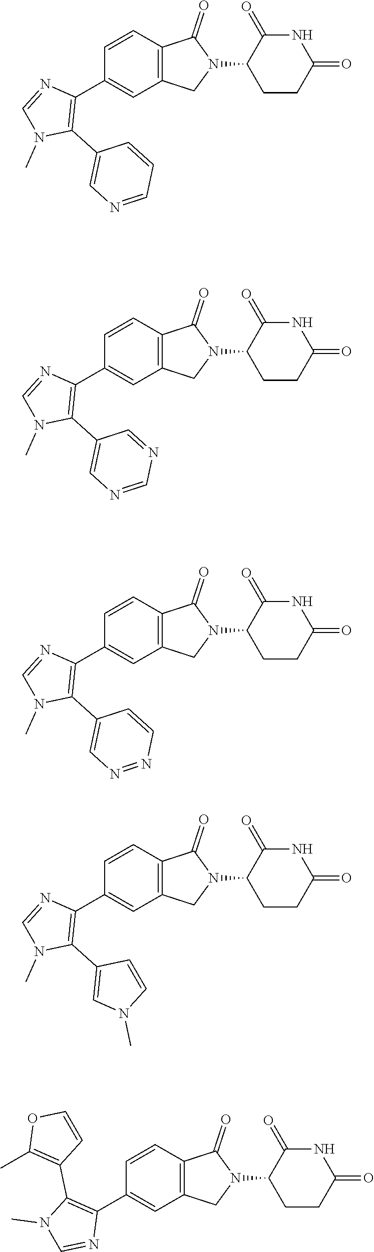 [SMILES: Cn1cnc(-c2ccc3c(c2)CN([C@H]2CCC(=O)NC2=O)C3=O)c1-c1cccnc1 ; Cn1cnc(-c2ccc3c(c2)CN([C@H]2CCC(=O)NC2=O)C3=O)c1-c1cncnc1 ; Cn1cnc(-c2ccc3c(c2)CN([C@H]2CCC(=O)NC2=O)C3=O)c1-c1ccnnc1 ; Cn1ccc(-c2c(-c3ccc4c(c3)CN([C@H]3CCC(=O)NC3=O)C4=O)ncn2C)c1 ; Cc1occc1-c1c(-c2ccc3c(c2)CN([C@H]2CCC(=O)NC2=O)C3=O)ncn1C]

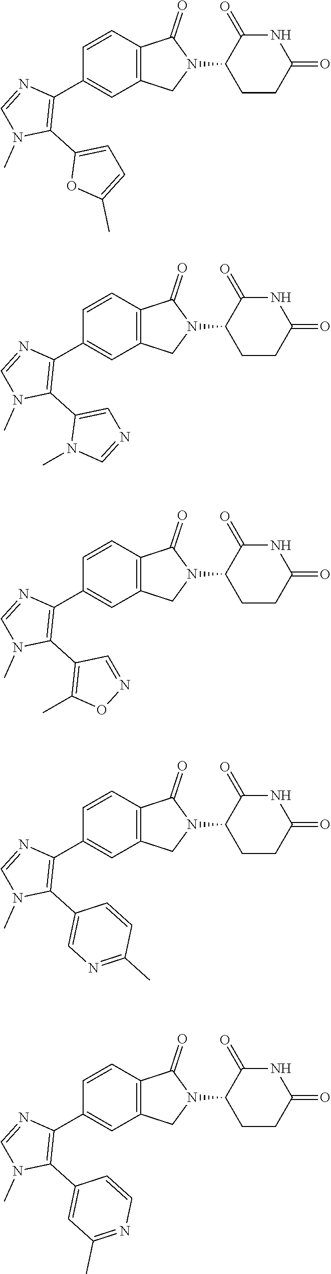 [SMILES: Cc1ccc(-c2c(-c3ccc4c(c3)CN([C@H]3CCC(=O)NC3=O)C4=O)ncn2C)o1 ; Cn1cncc1-c1c(-c2ccc3c(c2)CN([C@H]2CCC(=O)NC2=O)C3=O)ncn1C ; Cc1oncc1-c1c(-c2ccc3c(c2)CN([C@H]2CCC(=O)NC2=O)C3=O)ncn1C ; Cc1ccc(-c2c(-c3ccc4c(c3)CN([C@H]3CCC(=O)NC3=O)C4=O)ncn2C)cn1 ; Cc1cc(-c2c(-c3ccc4c(c3)CN([C@H]3CCC(=O)NC3=O)C4=O)ncn2C)ccn1]

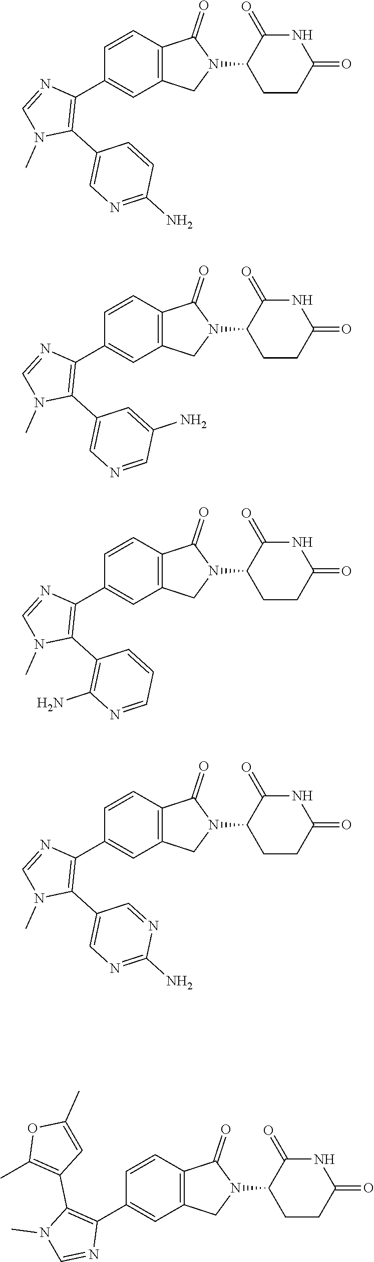 [SMILES: Cn1cnc(-c2ccc3c(c2)CN([C@H]2CCC(=O)NC2=O)C3=O)c1-c1ccc(N)nc1 ; Cn1cnc(-c2ccc3c(c2)CN([C@H]2CCC(=O)NC2=O)C3=O)c1-c1cncc(N)c1 ; Cn1cnc(-c2ccc3c(c2)CN([C@H]2CCC(=O)NC2=O)C3=O)c1-c1cccnc1N ; Cn1cnc(-c2ccc3c(c2)CN([C@H]2CCC(=O)NC2=O)C3=O)c1-c1cnc(N)nc1 ; Cc1cc(-c2c(-c3ccc4c(c3)CN([C@H]3CCC(=O)NC3=O)C4=O)ncn2C)c(C)o1]

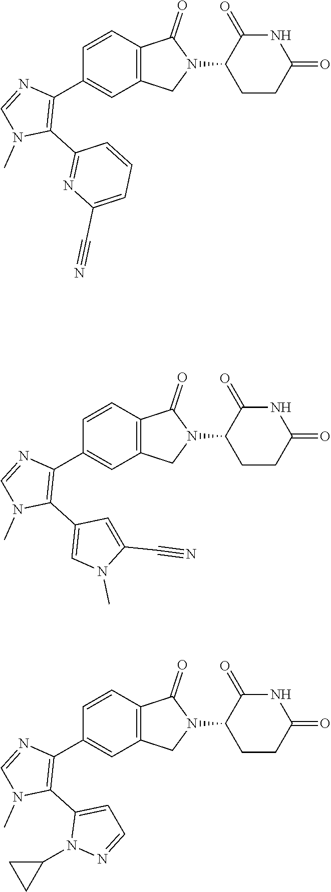 [SMILES: Cn1cnc(-c2ccc3c(c2)CN([C@H]2CCC(=O)NC2=O)C3=O)c1-c1cccc(C#N)n1 ; Cn1cc(-c2c(-c3ccc4c(c3)CN([C@H]3CCC(=O)NC3=O)C4=O)ncn2C)cc1C#N ; Cn1cnc(-c2ccc3c(c2)CN([C@H]2CCC(=O)NC2=O)C3=O)c1-c1ccnn1C1CC1]

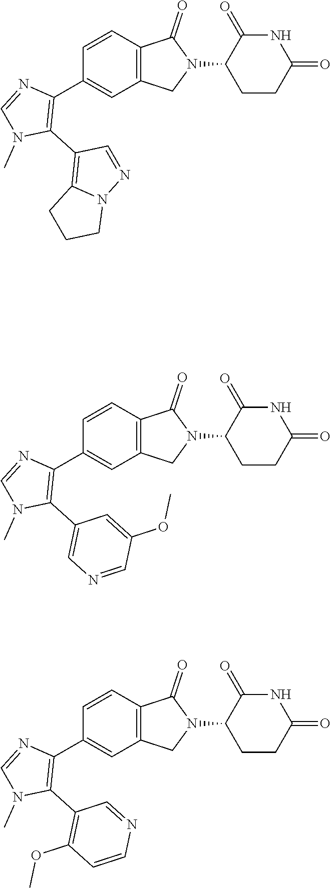 [SMILES: Cn1cnc(-c2ccc3c(c2)CN([C@H]2CCC(=O)NC2=O)C3=O)c1-c1cnn2c1CCC2 ; COc1cncc(-c2c(-c3ccc4c(c3)CN([C@H]3CCC(=O)NC3=O)C4=O)ncn2C)c1 ; COc1ccncc1-c1c(-c2ccc3c(c2)CN([C@H]2CCC(=O)NC2=O)C3=O)ncn1C]

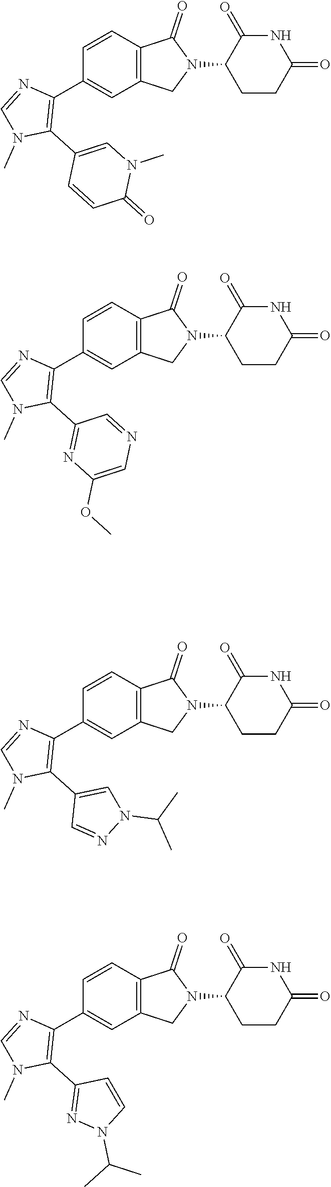 [SMILES: Cn1cnc(-c2ccc3c(c2)CN([C@H]2CCC(=O)NC2=O)C3=O)c1-c1ccc(=O)n(C)c1 ; COc1cncc(-c2c(-c3ccc4c(c3)CN([C@H]3CCC(=O)NC3=O)C4=O)ncn2C)n1 ; CC(C)n1cc(-c2c(-c3ccc4c(c3)CN([C@H]3CCC(=O)NC3=O)C4=O)ncn2C)cn1 ; CC(C)n1ccc(-c2c(-c3ccc4c(c3)CN([C@H]3CCC(=O)NC3=O)C4=O)ncn2C)n1]

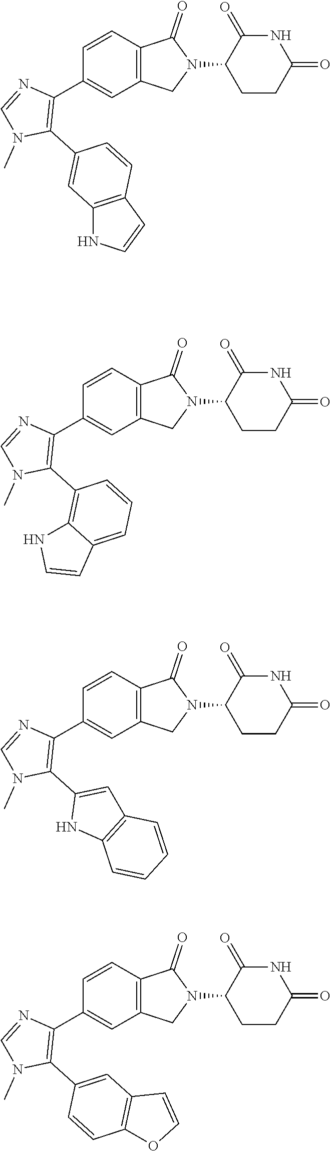 [SMILES: Cn1cnc(-c2ccc3c(c2)CN([C@H]2CCC(=O)NC2=O)C3=O)c1-c1ccc2cc[nH]c2c1 ; Cn1cnc(-c2ccc3c(c2)CN([C@H]2CCC(=O)NC2=O)C3=O)c1-c1cccc2cc[nH]c12 ; Cn1cnc(-c2ccc3c(c2)CN([C@H]2CCC(=O)NC2=O)C3=O)c1-c1cc2ccccc2[nH]1 ; Cn1cnc(-c2ccc3c(c2)CN([C@H]2CCC(=O)NC2=O)C3=O)c1-c1ccc2occc2c1]

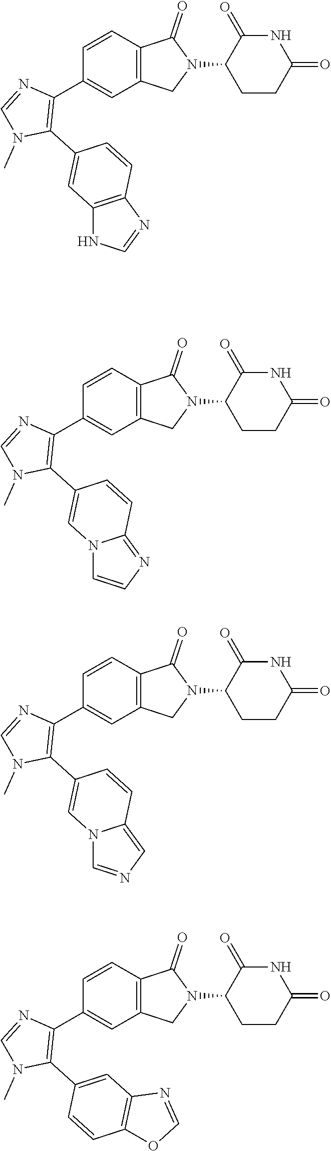 [SMILES: Cn1cnc(-c2ccc3c(c2)CN([C@H]2CCC(=O)NC2=O)C3=O)c1-c1ccc2nc[nH]c2c1 ; Cn1cnc(-c2ccc3c(c2)CN([C@H]2CCC(=O)NC2=O)C3=O)c1-c1ccc2nccn2c1 ; Cn1cnc(-c2ccc3c(c2)CN([C@H]2CCC(=O)NC2=O)C3=O)c1-c1ccc2cncn2c1 ; Cn1cnc(-c2ccc3c(c2)CN([C@H]2CCC(=O)NC2=O)C3=O)c1-c1ccc2ocnc2c1]

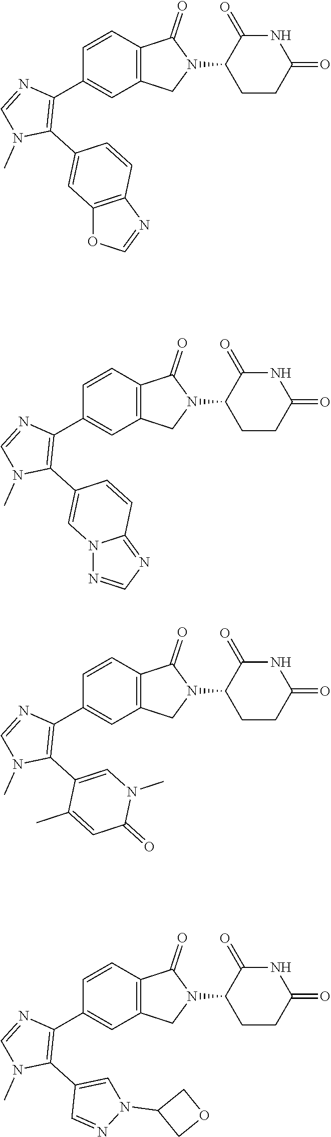 [SMILES: Cn1cnc(-c2ccc3c(c2)CN([C@H]2CCC(=O)NC2=O)C3=O)c1-c1ccc2ncoc2c1 ; Cn1cnc(-c2ccc3c(c2)CN([C@H]2CCC(=O)NC2=O)C3=O)c1-c1ccc2ncnn2c1 ; Cc1cc(=O)n(C)cc1-c1c(-c2ccc3c(c2)CN([C@H]2CCC(=O)NC2=O)C3=O)ncn1C ; Cn1cnc(-c2ccc3c(c2)CN([C@H]2CCC(=O)NC2=O)C3=O)c1-c1cnn(C2COC2)c1]

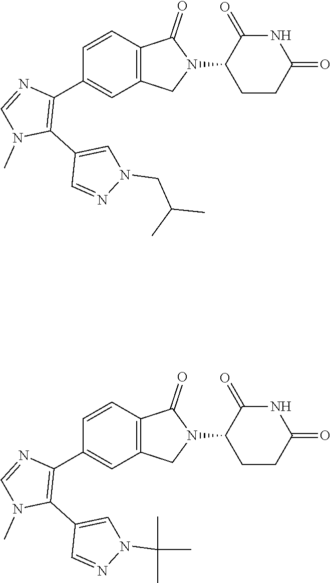 [SMILES: CC(C)Cn1cc(-c2c(-c3ccc4c(c3)CN([C@H]3CCC(=O)NC3=O)C4=O)ncn2C)cn1 ; Cn1cnc(-c2ccc3c(c2)CN([C@H]2CCC(=O)NC2=O)C3=O)c1-c1cnn(C(C)(C)C)c1]

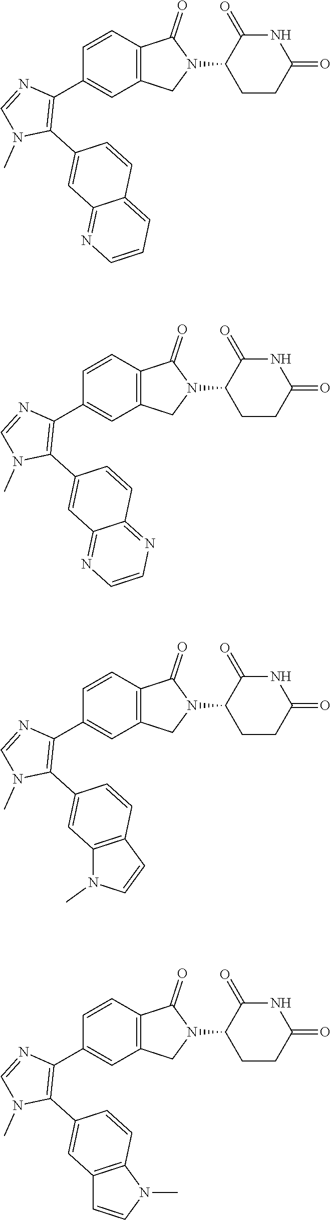 [SMILES: Cn1cnc(-c2ccc3c(c2)CN([C@H]2CCC(=O)NC2=O)C3=O)c1-c1ccc2cccnc2c1 ; Cn1cnc(-c2ccc3c(c2)CN([C@H]2CCC(=O)NC2=O)C3=O)c1-c1ccc2nccnc2c1 ; Cn1cnc(-c2ccc3c(c2)CN([C@H]2CCC(=O)NC2=O)C3=O)c1-c1ccc2ccn(C)c2c1 ; Cn1cnc(-c2ccc3c(c2)CN([C@H]2CCC(=O)NC2=O)C3=O)c1-c1ccc2c(ccn2C)c1]

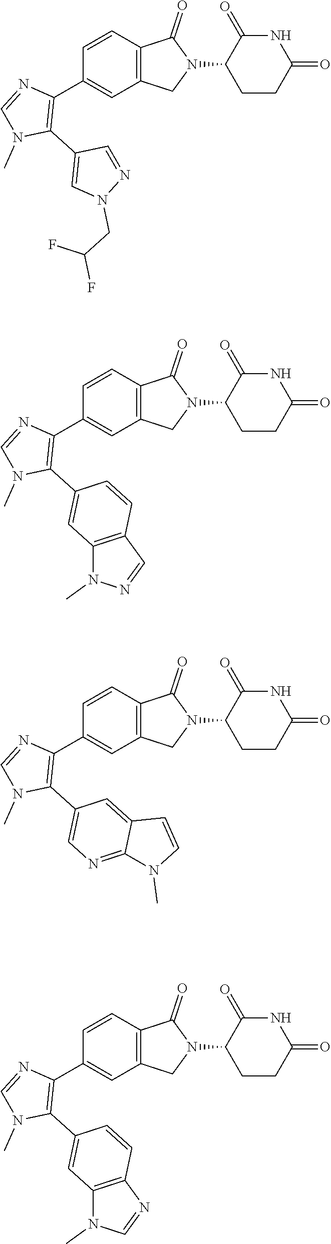 [SMILES: Cn1cnc(-c2ccc3c(c2)CN([C@H]2CCC(=O)NC2=O)C3=O)c1-c1cnn(CC(F)F)c1 ; Cn1cnc(-c2ccc3c(c2)CN([C@H]2CCC(=O)NC2=O)C3=O)c1-c1ccc2cnn(C)c2c1 ; Cn1cnc(-c2ccc3c(c2)CN([C@H]2CCC(=O)NC2=O)C3=O)c1-c1cnc2c(ccn2C)c1 ; Cn1cnc(-c2ccc3c(c2)CN([C@H]2CCC(=O)NC2=O)C3=O)c1-c1ccc2ncn(C)c2c1]

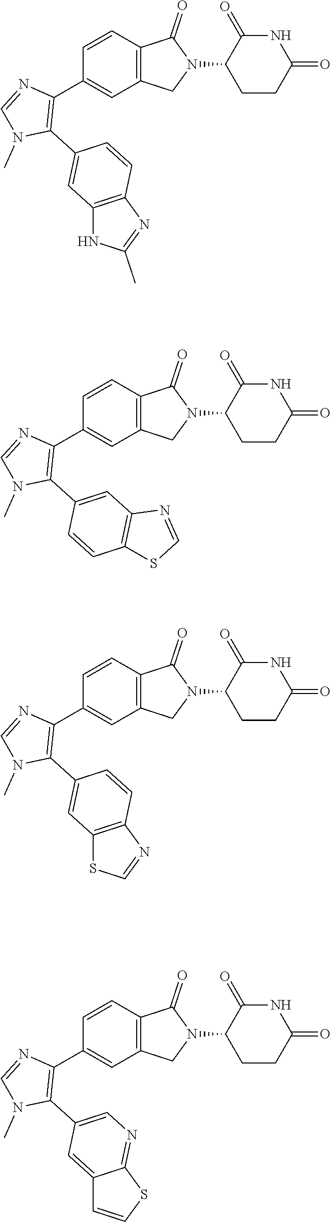 [SMILES: Cc1nc2ccc(-c3c(-c4ccc5c(c4)CN([C@H]4CCC(=O)NC4=O)C5=O)ncn3C)cc2[nH]1 ; Cn1cnc(-c2ccc3c(c2)CN([C@H]2CCC(=O)NC2=O)C3=O)c1-c1ccc2scnc2c1 ; Cn1cnc(-c2ccc3c(c2)CN([C@H]2CCC(=O)NC2=O)C3=O)c1-c1ccc2ncsc2c1 ; Cn1cnc(-c2ccc3c(c2)CN([C@H]2CCC(=O)NC2=O)C3=O)c1-c1cnc2sccc2c1]

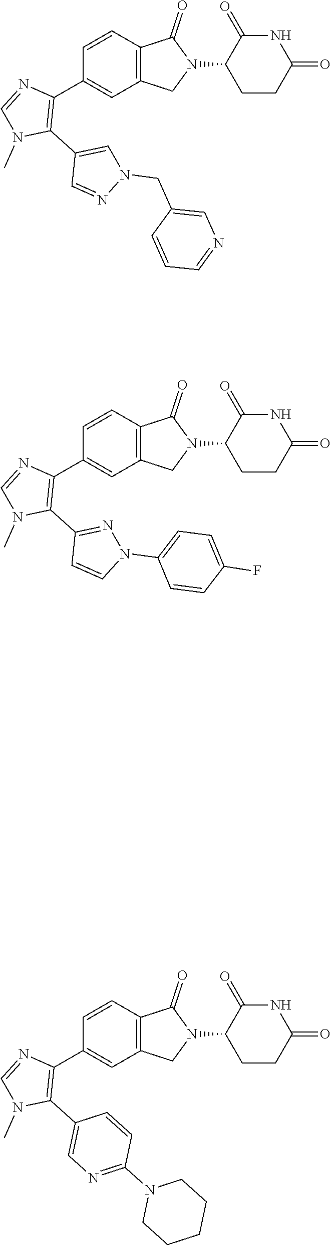 [SMILES: Cn1cnc(-c2ccc3c(c2)CN([C@H]2CCC(=O)NC2=O)C3=O)c1-c1cnn(Cc2cccnc2)c1 ; Cn1cnc(-c2ccc3c(c2)CN([C@H]2CCC(=O)NC2=O)C3=O)c1-c1ccn(-c2ccc(F)cc2)n1 ; Cn1cnc(-c2ccc3c(c2)CN([C@H]2CCC(=O)NC2=O)C3=O)c1-c1ccc(N2CCCCC2)nc1]

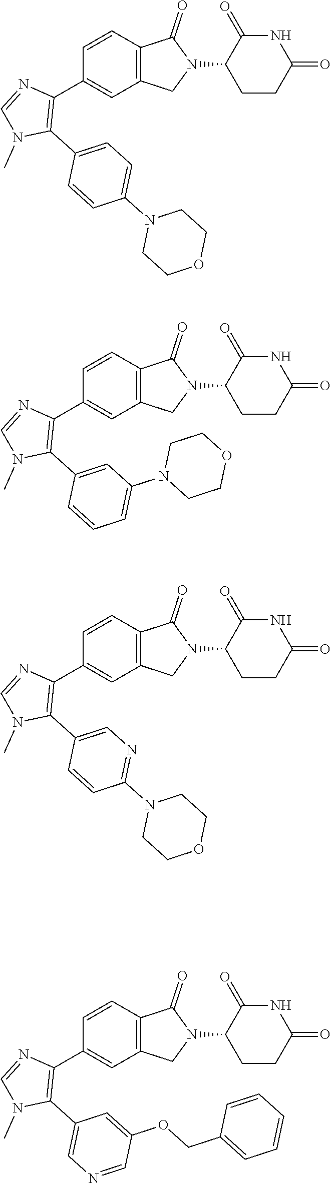 [SMILES: Cn1cnc(-c2ccc3c(c2)CN([C@H]2CCC(=O)NC2=O)C3=O)c1-c1ccc(N2CCOCC2)cc1 ; Cn1cnc(-c2ccc3c(c2)CN([C@H]2CCC(=O)NC2=O)C3=O)c1-c1cccc(N2CCOCC2)c1 ; Cn1cnc(-c2ccc3c(c2)CN([C@H]2CCC(=O)NC2=O)C3=O)c1-c1ccc(N2CCOCC2)nc1 ; Cn1cnc(-c2ccc3c(c2)CN([C@H]2CCC(=O)NC2=O)C3=O)c1-c1cncc(OCc2ccccc2)c1]

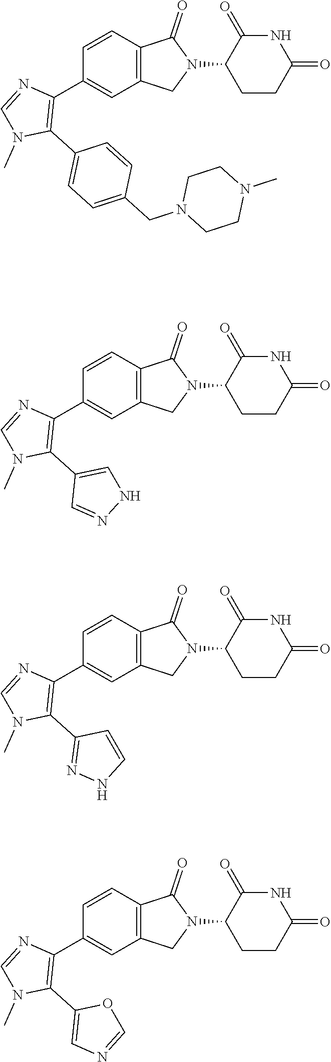 [SMILES: CN1CCN(Cc2ccc(-c3c(-c4ccc5c(c4)CN([C@H]4CCC(=O)NC4=O)C5=O)ncn3C)cc2)CC1 ; Cn1cnc(-c2ccc3c(c2)CN([C@H]2CCC(=O)NC2=O)C3=O)c1-c1cn[nH]c1 ; Cn1cnc(-c2ccc3c(c2)CN([C@H]2CCC(=O)NC2=O)C3=O)c1-c1cc[nH]n1 ; Cn1cnc(-c2ccc3c(c2)CN([C@H]2CCC(=O)NC2=O)C3=O)c1-c1cnco1]

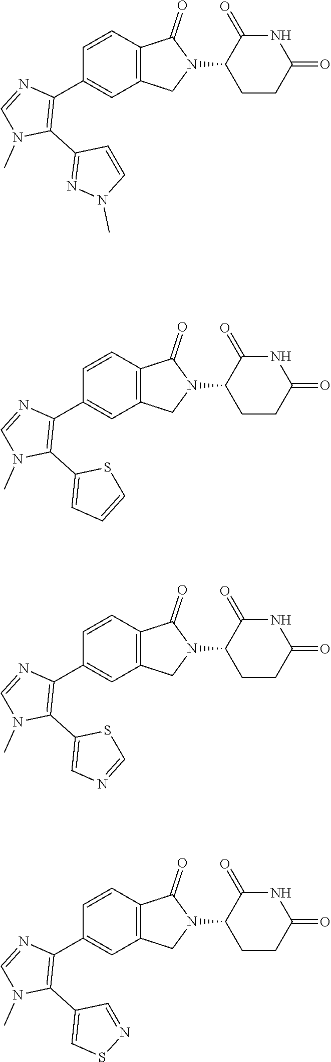 [SMILES: Cn1ccc(-c2c(-c3ccc4c(c3)CN([C@H]3CCC(=O)NC3=O)C4=O)ncn2C)n1 ; Cn1cnc(-c2ccc3c(c2)CN([C@H]2CCC(=O)NC2=O)C3=O)c1-c1cccs1 ; Cn1cnc(-c2ccc3c(c2)CN([C@H]2CCC(=O)NC2=O)C3=O)c1-c1cncs1 ; Cn1cnc(-c2ccc3c(c2)CN([C@H]2CCC(=O)NC2=O)C3=O)c1-c1cnsc1]

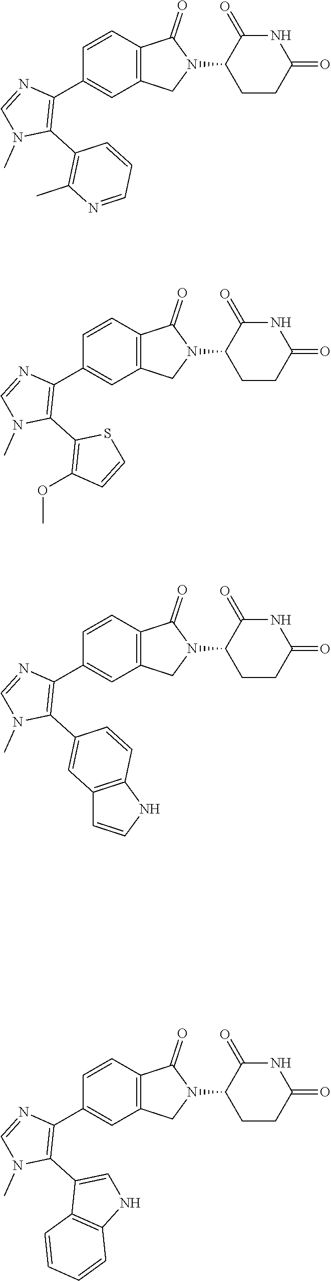 [SMILES: Cc1ncccc1-c1c(-c2ccc3c(c2)CN([C@H]2CCC(=O)NC2=O)C3=O)ncn1C ; COc1ccsc1-c1c(-c2ccc3c(c2)CN([C@H]2CCC(=O)NC2=O)C3=O)ncn1C ; Cn1cnc(-c2ccc3c(c2)CN([C@H]2CCC(=O)NC2=O)C3=O)c1-c1ccc2[nH]ccc2c1 ; Cn1cnc(-c2ccc3c(c2)CN([C@H]2CCC(=O)NC2=O)C3=O)c1-c1c[nH]c2ccccc12]

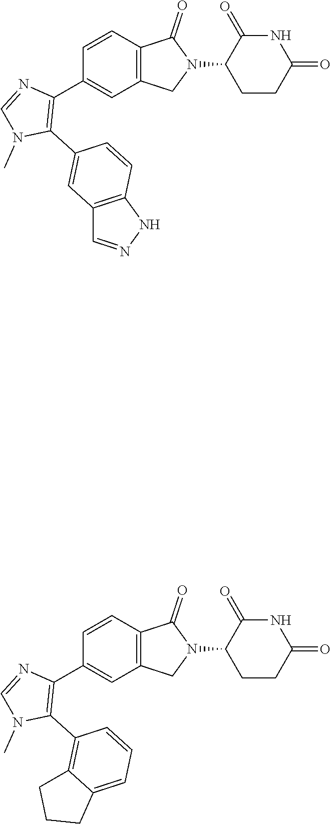 [SMILES: Cn1cnc(-c2ccc3c(c2)CN([C@H]2CCC(=O)NC2=O)C3=O)c1-c1ccc2[nH]ncc2c1 ; Cn1cnc(-c2ccc3c(c2)CN([C@H]2CCC(=O)NC2=O)C3=O)c1-c1cccc2c1CCC2]

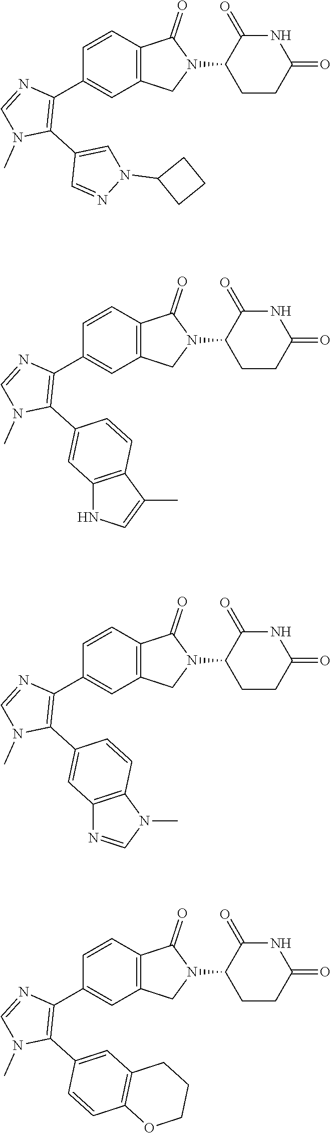 [SMILES: Cn1cnc(-c2ccc3c(c2)CN([C@H]2CCC(=O)NC2=O)C3=O)c1-c1cnn(C2CCC2)c1 ; Cc1c[nH]c2cc(-c3c(-c4ccc5c(c4)CN([C@H]4CCC(=O)NC4=O)C5=O)ncn3C)ccc12 ; Cn1cnc(-c2ccc3c(c2)CN([C@H]2CCC(=O)NC2=O)C3=O)c1-c1ccc2c(c1)ncn2C ; Cn1cnc(-c2ccc3c(c2)CN([C@H]2CCC(=O)NC2=O)C3=O)c1-c1ccc2c(c1)CCCO2]

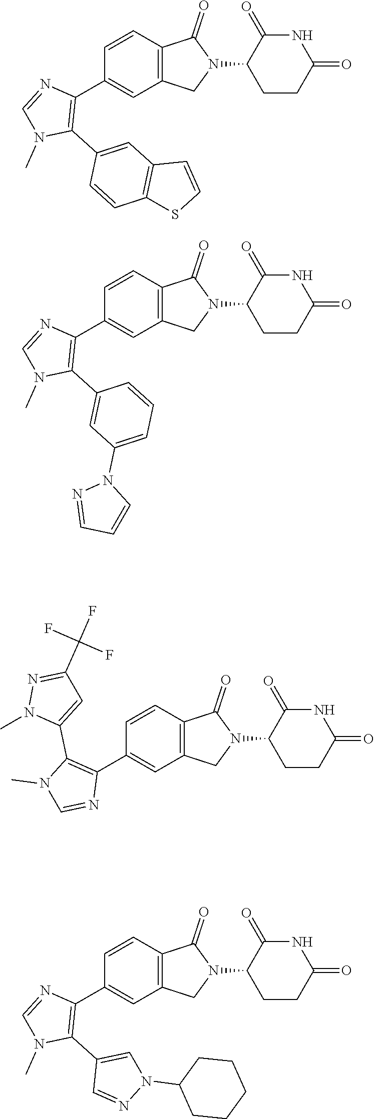

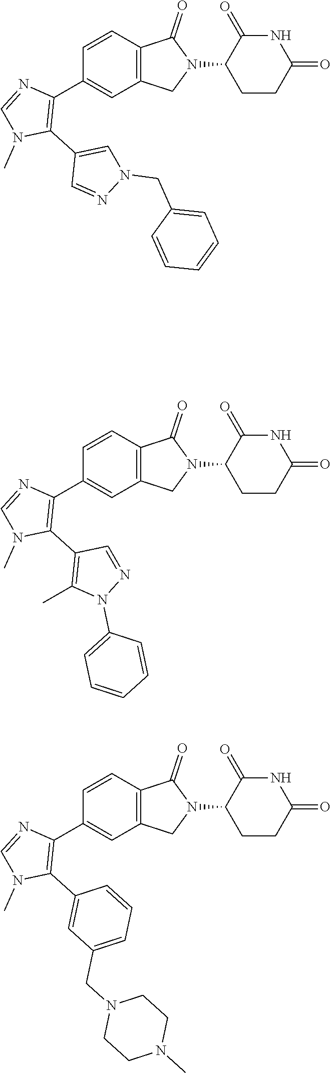 [SMILES: Cn1cnc(-c2ccc3c(c2)CN([C@H]2CCC(=O)NC2=O)C3=O)c1-c1cnn(Cc2ccccc2)c1 ; Cc1c(-c2c(-c3ccc4c(c3)CN([C@H]3CCC(=O)NC3=O)C4=O)ncn2C)cnn1-c1ccccc1 ; CN1CCN(Cc2cccc(-c3c(-c4ccc5c(c4)CN([C@H]4CCC(=O)NC4=O)C5=O)ncn3C)c2)CC1]

### III. Synthesis of the Compounds

The compounds provided herein may be synthesized using standard methods well known to those of skill in the art starting with commercially available starting materials. In one embodiment, the compound provided herein is synthesized according to one of the methods shown below.

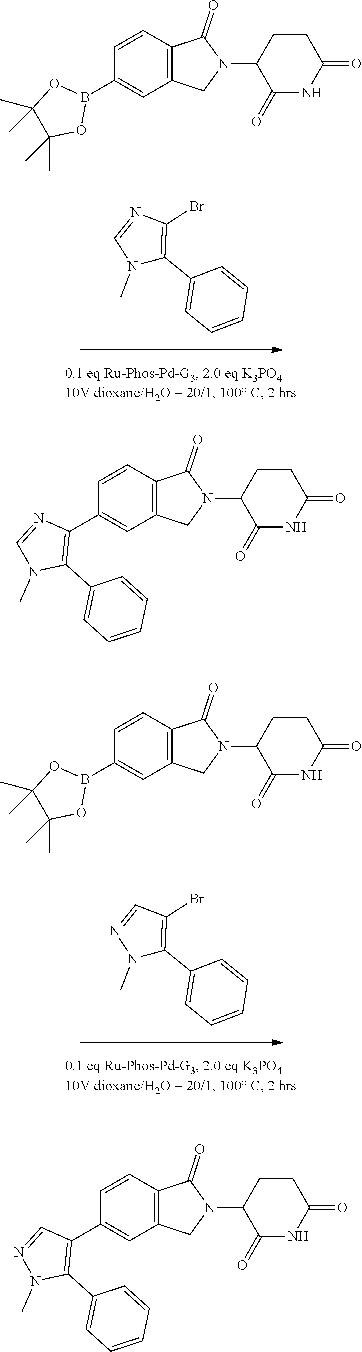 [SMILES: CC1(C)OB(c2ccc3c(c2)CN(C2CCC(=O)NC2=O)C3=O)OC1(C)C ; Cn1cnc(Br)c1-c1ccccc1 ; Cn1cnc(-c2ccc3c(c2)CN(C2CCC(=O)NC2=O)C3=O)c1-c1ccccc1 ; CC1(C)OB(c2ccc3c(c2)CN(C2CCC(=O)NC2=O)C3=O)OC1(C)C ; Cn1ncc(Br)c1-c1ccccc1 ; Cn1ncc(-c2ccc3c(c2)CN(C2CCC(=O)NC2=O)C3=O)c1-c1ccccc1]

In another embodiment, a library of compounds may be synthesized according to the method shown below (see, e.g., WO 2021/226269, WO 2020/127685, WO 2010/068242):

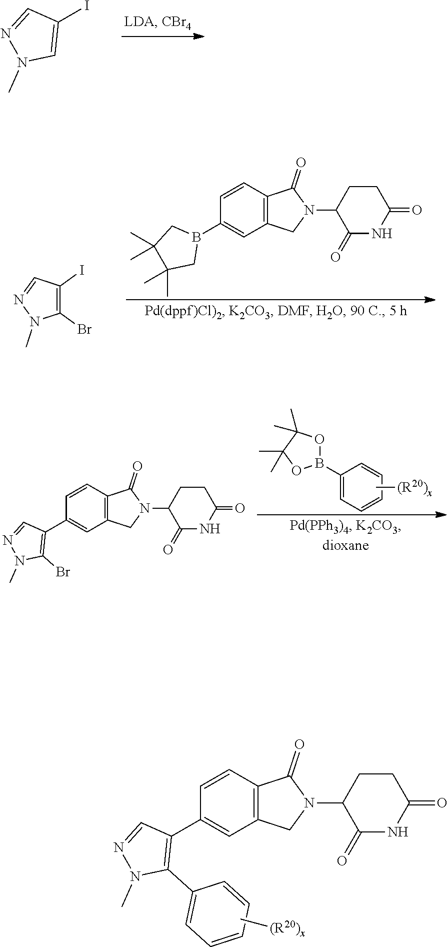

where R²⁰ is a substituent on Ar, as defined herein, and x is an integer from 1 to 5, or from 1 to 3, or 1 or 2.

In another embodiment, a library of compounds may be synthesized according to the method shown below (see, e.g., WO 2014/151945, WO 2010 068242):

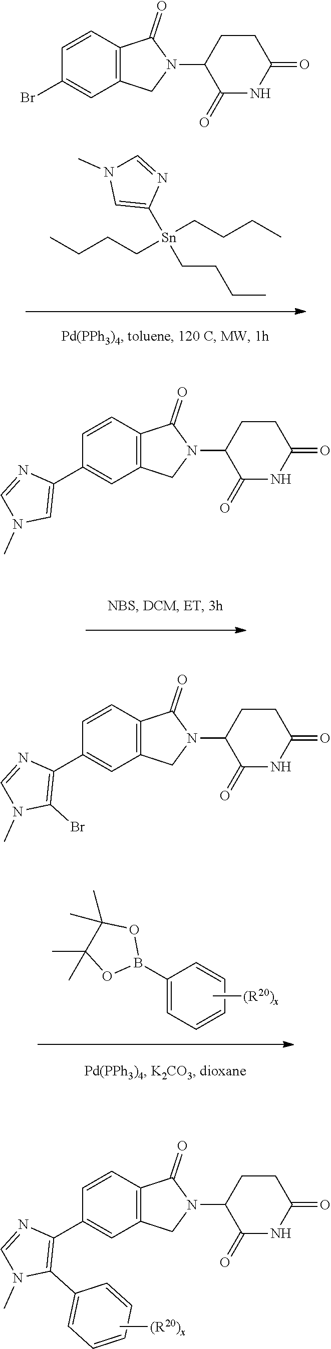

where R²⁰ is a substituent on Ar, as defined herein, and x is an integer from 1 to 5, or from 1 to 3, or 1 or 2.

In another embodiment, a library of compounds may be synthesized according to the method shown below (see, e.g., WO 2014/151945, WO 2010 068242):

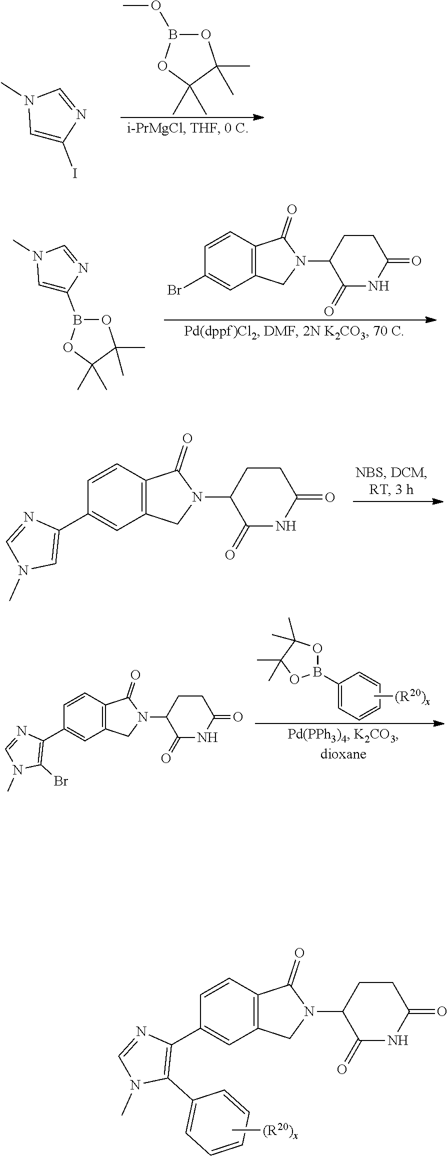

where R²⁰ is a substituent on Ar, as defined herein, and x is an integer from 1 to 5, or from 1 to 3, or 1 or 2.

In another embodiment, a library of compounds may be synthesized according to one of the methods shown below:

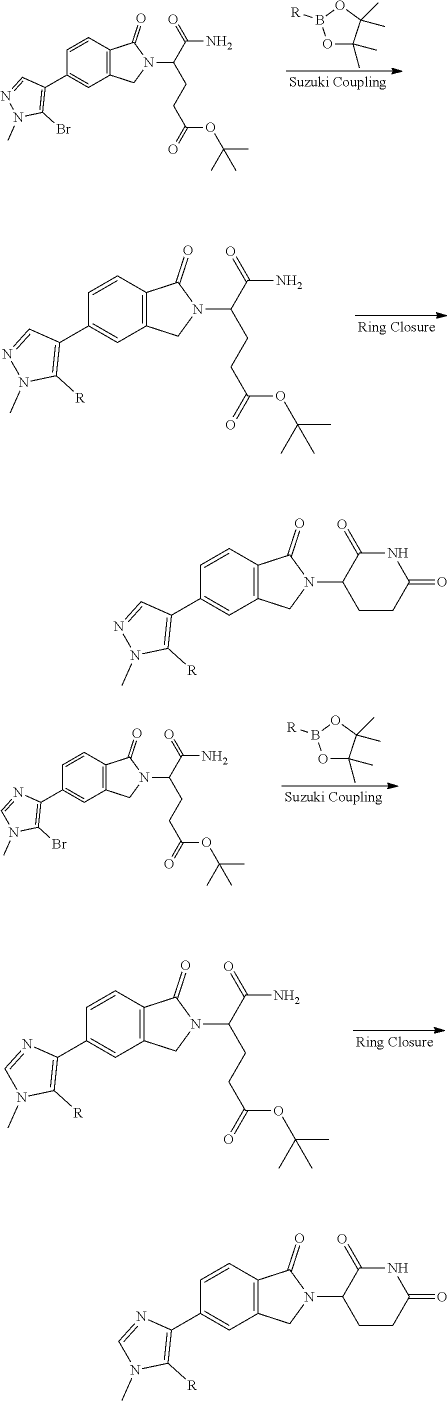

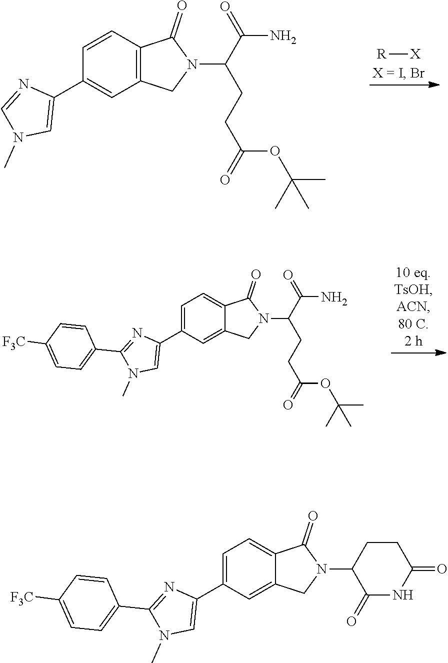

where R is Ar as defined herein.

### IV. Pharmaceutical Compositions

The pharmaceutical compositions provided herein contain therapeutically effective amounts of one or more of compounds provided herein and a pharmaceutically acceptable carrier, diluent or excipient.

The compounds can be formulated into suitable pharmaceutical preparations such as solutions, suspensions, tablets, dispersible tablets, pills, capsules, powders, sustained release formulations or elixirs, for oral administration or in sterile solutions or suspensions for ophthalmic or parenteral administration, as well as transdermal patch preparation and dry powder inhalers. Typically, the compounds described above are formulated into pharmaceutical compositions using techniques and procedures well known in the art (see, e.g., Ansel Introduction to Pharmaceutical Dosage Forms, Seventh Edition 1999).

In the compositions, effective concentrations of one or more compounds or pharmaceutically acceptable salts is (are) mixed with a suitable pharmaceutical carrier or vehicle. In certain embodiments, the concentrations of the compounds in the compositions are effective for delivery of an amount, upon administration, that treats, prevents, or ameliorates one or more of the symptoms and/or progression of a disease or disorder disclosed herein.

Typically, the compositions are formulated for single dosage administration. To formulate a composition, the weight fraction of compound is dissolved, suspended, dispersed or otherwise mixed in a selected vehicle at an effective concentration such that the treated condition is relieved or ameliorated. Pharmaceutical carriers or vehicles suitable for administration of the compounds provided herein include any such carriers known to those skilled in the art to be suitable for the particular mode of administration.

In addition, the compounds may be formulated as the sole pharmaceutically active ingredient in the composition or may be combined with other active ingredients. Liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as known in the art. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

The active compound is included in the pharmaceutically acceptable carrier in an amount sufficient to exert a therapeutically useful effect in the absence of undesirable side effects on the subject treated. The therapeutically effective concentration may be determined empirically by testing the compounds in in vitro and in vivo systems described herein and then extrapolated therefrom for dosages for humans. In some embodiments, the active compound is administered in a method to achieve a therapeutically effective concentration of the drug. In some embodiments, a companion diagnostic (see, e.g., Olsen D and Jorgensen J T, Front. Oncol., 2014 May 16, 4:105, doi: 10.3389/fonC.2014.00105) is used to determine the therapeutic concentration and safety profile of the active compound in specific subjects or subject populations.

The concentration of active compound in the pharmaceutical composition will depend on absorption, tissue distribution, inactivation and excretion rates of the active compound, the physicochemical characteristics of the compound, the dosage schedule, and amount administered as well as other factors known to those of skill in the art. For example, the amount that is delivered is sufficient to ameliorate one or more of the symptoms of a disease or disorder disclosed herein.

In certain embodiments, a therapeutically effective dosage should produce a serum concentration of active ingredient of from about 0.1 ng/mL to about 50-100 μg/mL. In one embodiment, the pharmaceutical compositions provide a dosage of from about 0.001 mg to about 2000 mg of compound per kilogram of body weight per day. Pharmaceutical dosage unit forms are prepared to provide from about 1 mg to about 1000 mg and in certain embodiments, from about 10 to about 500 mg of the essential active ingredient or a combination of essential ingredients per dosage unit form.

The active ingredient may be administered at once or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Thus, effective concentrations or amounts of one or more of the compounds described herein or pharmaceutically acceptable salts thereof are mixed with a suitable pharmaceutical carrier or vehicle for systemic, topical or local administration to form pharmaceutical compositions. Compounds are included in an amount effective for ameliorating one or more symptoms of, or for treating, retarding progression, or preventing. The concentration of active compound in the composition will depend on absorption, tissue distribution, inactivation, excretion rates of the active compound, the dosage schedule, amount administered, particular formulation as well as other factors known to those of skill in the art.

The compositions are intended to be administered by a suitable route, including but not limited to oral, parenteral, subcutaneous, intravenous, intramuscular, intraperitoneal, intrathecal, mucosal, dermal, transdermal, buccal, rectal, topical, local, nasal or inhalation. For oral administration, capsules and tablets can be formulated. The compositions are in liquid, semi-liquid or solid form and are formulated in a manner suitable for each route of administration.

Solutions or suspensions used for parenteral, intradermal, subcutaneous, or topical application can include any of the following components: a sterile diluent, such as water for injection, saline solution, fixed oil, polyethylene glycol, glycerin, propylene glycol, dimethyl acetamide or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampules, pens, disposable syringes or single or multiple dose vials made of glass, plastic or other suitable material.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolution in aqueous sodium bicarbonate.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil water emulsions containing suitable quantities of the compounds or pharmaceutically acceptable salts thereof. The pharmaceutically therapeutically active compounds and salts thereof are formulated and administered in unit dosage forms or multiple dosage forms. Unit dose forms as used herein refer to physically discrete units suitable for human and animal subjects and packaged individually as is known in the art. Each unit dose contains a predetermined quantity of the therapeutically active compound sufficient to produce the desired therapeutic effect, in association with the required pharmaceutical carrier, vehicle or diluent. Examples of unit dose forms include ampules and syringes and individually packaged tablets or capsules. Unit dose forms may be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, multiple dose form is a multiple of unit doses which are not segregated in packaging.

Sustained-release preparations can also be prepared. Suitable examples of sustained-release preparations include semipermeable matrices of solid hydrophobic polymers containing the compound provided herein, which matrices are in the form of shaped articles, e.g., films, or microcapsule. Examples of sustained-release matrices include iontophoresis patches, polyesters, hydrogels (for example, poly(2-hydroxyethyl-methacrylate), or poly(vinylalcohol)), polylactides, copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated compound remain in the body for a long time, they may denature or aggregate as a result of exposure to moisture at 37° C., resulting in a loss of biological activity and possible changes in their structure. Rational strategies can be devised for stabilization depending on the mechanism of action involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Dosage forms or compositions containing active ingredient in the range of 0.005% to 100% with the balance made up from non-toxic carrier may be prepared. For oral administration, a pharmaceutically acceptable non-toxic composition is formed by the incorporation of any of the normally employed excipients, such as, for example pharmaceutical grades of mannitol, lactose, starch, magnesium stearate, talcum, cellulose derivatives, sodium croscarmellose, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of these compositions are known to those skilled in the art. The contemplated compositions may contain about 0.001% 100% active ingredient, in certain embodiments, about 0.1 85% about 75-95%.

The active compounds or pharmaceutically acceptable salts may be prepared with carriers that protect the compound against rapid elimination from the body, such as time release formulations or coatings.

The compositions may include other active compounds to obtain desired combinations of properties. The compounds provided herein, or pharmaceutically acceptable salts thereof as described herein, may also be advantageously administered for therapeutic or prophylactic purposes together with another pharmacological agent known in the general art to be of value in treating one or more of the diseases or medical conditions referred to hereinabove, such as diseases related to oxidative stress. It is to be understood that such combination therapy constitutes a further aspect of the compositions and methods of treatment provided herein.

Lactose-free compositions provided herein can contain excipients that are well known in the art and are listed, for example, in the U.S. Pharmacopeia (USP) SP (XXI)/NF (XVI). In general, lactose-free compositions contain an active ingredient, a binder/filler, and a lubricant in pharmaceutically compatible and pharmaceutically acceptable amounts. Exemplary lactose-free dosage forms contain an active ingredient, microcrystalline cellulose, pre-gelatinized starch and magnesium stearate.

Further encompassed are anhydrous pharmaceutical compositions and dosage forms containing a compound provided herein. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment and use of formulations.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine are anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e.g., vials), blister packs and strip packs.

### A. Oral Dosage Forms

Oral pharmaceutical dosage forms are either solid, gel or liquid. The solid dosage forms are tablets, capsules, granules, and bulk powders. Types of oral tablets include compressed, chewable lozenges and tablets which may be enteric coated, sugar coated or film coated. Capsules may be hard or soft gelatin capsules, while granules and powders may be provided in non-effervescent or effervescent form with the combination of other ingredients known to those skilled in the art.

In certain embodiments, the formulations are solid dosage forms, such as capsules or tablets. The tablets, pills, capsules, troches and the like can contain any of the following ingredients, or compounds of a similar nature: a binder; a diluent; a disintegrating agent; a lubricant; a glidant; a sweetening agent; and a flavoring agent.

Examples of binders include microcrystalline cellulose, gum tragacanth, glucose solution, acacia mucilage, gelatin solution, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include croscarmellose sodium, sodium starch glycolate, crospovidone, alginic acid, corn starch, potato starch, bentonite, methylcellulose, agar and carboxymethylcellulose. Coloring agents include, for example, any of the approved certified water-soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene lauryl ether. Emetic coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

If oral administration is desired, the compound could be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a compound or pharmaceutically acceptable salt thereof as described herein. Higher concentrations, up to about 98% by weight of the active ingredient may be included.

Pharmaceutically acceptable carriers included in tablets are binders, lubricants, diluents, disintegrating agents, coloring agents, flavoring agents, and wetting agents. Enteric coated tablets, because of the enteric coating, resist the action of stomach acid and dissolve or disintegrate in the neutral or alkaline intestines. Sugar coated tablets are compressed tablets to which different layers of pharmaceutically acceptable substances are applied. Film coated tablets are compressed tablets which have been coated with a polymer or other suitable coating. Multiple compressed tablets are compressed tablets made by more than one compression cycle utilizing the pharmaceutically acceptable substances previously mentioned. Coloring agents may also be used in the above dosage forms. Flavoring and sweetening agents are used in compressed tablets, sugar coated, multiple compressed and chewable tablets. Flavoring and sweetening agents are especially useful in the formation of chewable tablets and lozenges.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil in-water or water in oil. In some embodiments, the suspension is a suspension of microparticles or nanoparticles. In some embodiments, the emulsion is an emulsion of microparticles or nanoparticles.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic add, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Diluents include lactose and sucrose. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic adds include citric and tartaric acid. Sources of carbon dioxide include sodium bicarbonate and sodium carbonate. Coloring agents include any of the approved certified water-soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include, but are not limited to, those containing a compound provided herein, a dialkylated mono- or poly-alkylene glycol, including, but not limited to, 1,2-dimethoxyethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

### B. Injectables, Solutions and Emulsions

Parenteral administration, generally characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. In some embodiments, the suspension is a suspension of microparticles or nanoparticles. In some embodiments, the emulsion is an emulsion of microparticles or nanoparticles. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow release or sustained release system, such that a constant level of dosage is maintained is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple dose containers which include phenols or cresols, mercurials, benzyl alcohol, chlorobutanol, methyl and propyl p hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the subject or animal as is known in the art.

The unit dose parenteral preparations are packaged in an ampule, a vial or a syringe with a needle. All preparations for parenteral administration must be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. Typically, a therapeutically effective dosage is formulated to contain a concentration of at least about 0.10% w/w up to about 90% w/w or more, such as more than 1% w/w of the active compound to the treated tissue(s). The active ingredient may be administered at once, or may be divided into a number of smaller doses to be administered at intervals of time. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

### C. Lyophilized Powders

Also provided herein are lyophilized powders, which can be reconstituted for administration as solutions, emulsions and other mixtures. They may also be reconstituted and formulated as solids or gels.

The sterile, lyophilized powder is prepared by dissolving a compound provided herein, or a pharmaceutically acceptable salt thereof, in a suitable solvent. The solvent may contain an excipient which improves the stability or other pharmacological component of the powder or reconstituted solution, prepared from the powder. Excipients that may be used include, but are not limited to, dextrose, sorbitol, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. Generally, the resulting solution will be apportioned into vials for lyophilization. Each vial will contain a single dosage (including but not limited to 10-1000 mg or 100-500 mg) or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, about 5-35 mg, or about 9-30 mg of lyophilized powder, is added per mL of sterile water or other suitable carrier. The precise amount depends upon the selected compound. Such amount can be empirically determined.

### D. Topical Administration

Topical mixtures are prepared as described for the local and systemic administration. The resulting mixture may be a solution, suspension, emulsion or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable salts thereof may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will have diameters of less than 50 microns or less than 10 microns.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

These solutions, particularly those intended for ophthalmic use, may be formulated as 0.01%-10% isotonic solutions, pH about 5-7, with appropriate salts.

### E. Compositions for Other Routes of Administration

Other routes of administration, such as topical application, transdermal patches, and rectal administration are also contemplated herein.

For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients.

Pharmaceutically acceptable substances utilized in rectal suppositories are bases or vehicles and agents to raise the melting point. Examples of bases include cocoa butter (theobroma oil), glycerin gelatin, carbowax (polyoxyethylene glycol) and appropriate mixtures of mono, di and triglycerides of fatty acids. Combinations of the various bases may be used. Agents to raise the melting point of suppositories include spermaceti and wax. Rectal suppositories may be prepared either by the compressed method or by molding. An exemplary weight of a rectal suppository is about 2 to 3 grams.

Tablets and capsules for rectal administration are manufactured using the same pharmaceutically acceptable substance and by the same methods as for formulations for oral administration.

### F. Sustained Release Compositions

Active ingredients provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, 5,639,480, 5,733,566, 5,739,108, 5,891,474, 5,922,356, 5,972,891, 5,980,945, 5,993,855, 6,045,830, 6,087,324, 6,113,943, 6,197,350, 6,248,363, 6,264,970, 6,267,981, 6,376,461, 6,419,961, 6,589,548, 6,613,358, 6,699,500 and 6,740,634, each of which is incorporated herein by reference. Such dosage forms can be used to provide slow or controlled-release of one or more active ingredients using, for example, hydroxypropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients provided herein.

All controlled-release pharmaceutical products have a common goal of improving drug therapy over that achieved by their non-controlled counterparts. In one embodiment, the use of an optimally designed controlled-release preparation in medical treatment is characterized by a minimum of drug substance being employed to cure or control the condition in a minimum amount of time. In certain embodiments, advantages of controlled-release formulations include extended activity of the drug, reduced dosage frequency, and increased subject compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled release of an active ingredient can be stimulated by various conditions including, but not limited to, pH, temperature, enzymes, water, or other physiological conditions or compounds.

In certain embodiments, the agent may be administered using intravenous infusion, an implantable osmotic pump, a transdermal patch, liposomes, or other modes of administration. In one embodiment, a pump may be used (see, Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In yet another embodiment, a controlled release system can be placed in proximity of the therapeutic target, i.e., thus requiring only a fraction of the systemic dose (see, e.g., Goodson, Medical Applications of Controlled Release, vol. 2, pp. 115-138 (1984).

In some embodiments, a controlled release device is introduced into a subject in proximity of the site of inappropriate immune activation or a tumor. Other controlled release systems are discussed in the review by Langer (Science 249:1527-1533 (1990). The active ingredient can be dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The active ingredient then diffuses through the outer polymeric membrane in a release rate controlling step. The percentage of active ingredient contained in such parenteral compositions is highly dependent on the specific nature thereof, as well as the needs of the subject.

### G. Targeted Formulations

The compounds provided herein, or pharmaceutically acceptable salts thereof, may also be formulated to be targeted to a particular tissue, receptor, or other area of the body of the subject to be treated, including liposome-, resealed erythrocyte-, and antibody-based delivery systems. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In one embodiment, the antibody-based delivery system is an antibody-drug conjugate (“ADC”), e.g., as described in Hamilton G S, Biologicals, 2015 September, 43(5):318-32; Kim E G and Kim K M, Biomol. Ther. (Seoul), 2015 November, 23(6):493-509; and Peters C and Brown S, Biosci. Rep., 2015 Jun. 12, 35(4) pii: e00225, each of which is incorporated herein by reference.

In one embodiment, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

### H. Articles of Manufacture

The compounds or pharmaceutically acceptable salts can be packaged as articles of manufacture containing packaging material, a compound or pharmaceutically acceptable salt thereof provided herein, which is used for treatment, prevention or amelioration of one or more symptoms or progression of a disease or disorder disclosed herein, and a label that indicates that the compound or pharmaceutically acceptable salt thereof is used for treatment, prevention or amelioration of one or more symptoms or progression of a disease or disorder disclosed herein.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, pens, bottles, and any packaging material suitable for a selected formulation and intended mode of administration and treatment. A wide array of formulations of the compounds and compositions provided herein are contemplated.

In certain embodiments, provided herein also are kits which, when used by the medical practitioner, can simplify the administration of appropriate amounts of active ingredients to a subject. In certain embodiments, the kit provided herein includes a container and a dosage form of a compound provided herein, including a single enantiomer or a mixture of diastereomers thereof; or a pharmaceutically acceptable salt, solvate, or prodrug thereof.

In certain embodiments, the kit includes a container comprising a dosage form of the compound provided herein, including a single enantiomer or a mixture of diastereomers thereof; or a pharmaceutically acceptable salt, solvate, or prodrug thereof, in a container comprising one or more other therapeutic agent(s) described herein.

Kits provided herein can further include devices that are used to administer the active ingredients. Examples of such devices include, but are not limited to, syringes, needle-less injectors drip bags, patches, and inhalers. The kits provided herein can also include condoms for administration of the active ingredients.

Kits provided herein can further include pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: aqueous vehicles, including, but not limited to, Water for Injection USP, Sodium Chloride Injection, Ringer's Injection, Dextrose Injection, Dextrose and Sodium Chloride Injection, and Lactated Ringer's Injection; water-miscible vehicles, including, but not limited to, ethyl alcohol, polyethylene glycol, and polypropylene glycol; and non-aqueous vehicles, including, but not limited to, corn oil, cottonseed oil, peanut oil, sesame oil, ethyl oleate, isopropyl myristate, and benzyl benzoate.

### V. Dosing

The compounds and pharmaceutical compositions provided herein may be dosed in certain therapeutically or prophylactically effective amounts, certain time intervals, certain dosage forms, and certain dosage administration methods as described below.

In certain embodiments, a therapeutically or prophylactically effective amount of the compound is from about 0.005 to about 1,000 mg per day, from about 0.01 to about 500 mg per day, from about 0.01 to about 250 mg per day, from about 0.01 to about 100 mg per day, from about 0.1 to about 100 mg per day, from about 0.5 to about 100 mg per day, from about 1 to about 100 mg per day, from about 0.01 to about 50 mg per day, from about 0.1 to about 50 mg per day, from about 0.5 to about 50 mg per day, from about 1 to about 50 mg per day, from about 0.02 to about 25 mg per day, from about 0.05 to about 10 mg per day, from about 0.05 to about 5 mg per day, from about 0.1 to about 5 mg per day, or from about 0.5 to about 5 mg per day.

In certain embodiments, the therapeutically or prophylactically effective amount is about 0.1, about 0.2, about 0.5, about 1, about 2, about 3, about 4, about 5, about 6, about 7, about 8, about 9, about 10, about 15, about 20, about 25, about 30, about 40, about 45, about 50, about 60, about 70, about 80, about 90, about 100, or about 150 mg per day.

In one embodiment, the recommended daily dose range of the compound provided herein, or a derivative thereof, for the conditions described herein lie within the range of from about 0.5 mg to about 50 mg per day, in one embodiment given as a single once-a-day dose, or in divided doses throughout a day. In some embodiments, the dosage ranges from about 1 mg to about 50 mg per day. In other embodiments, the dosage ranges from about 0.5 to about 5 mg per day. Specific doses per day include 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 mg per day.

In a specific embodiment, the recommended starting dosage may be 0.5, 1, 2, 3, 4, 5, 10, 15, 20, 25 or 50 mg per day. In another embodiment, the recommended starting dosage may be 0.5, 1, 2, 3, 4, or 5 mg per day. The dose may be escalated to 15, 20, 25, 30, 35, 40, 45 and 50 mg/day. In a specific embodiment, the compound can be administered in an amount of about 25 mg/day. In a particular embodiment, the compound can be administered in an amount of about 10 mg/day. In a particular embodiment, the compound can be administered in an amount of about 5 mg/day. In a particular embodiment, the compound can be administered in an amount of about 4 mg/day. In a particular embodiment, the compound can be administered in an amount of about 3 mg/day.

In certain embodiments, the therapeutically or prophylactically effective amount is from about 0.001 to about 100 mg/kg/day, from about 0.01 to about 50 mg/kg/day, from about 0.01 to about 25 mg/kg/day, from about 0.01 to about 10 mg/kg/day, from about 0.01 to about 9 mg/kg/day, 0.01 to about 8 mg/kg/day, from about 0.01 to about 7 mg/kg/day, from about 0.01 to about 6 mg/kg/day, from about 0.01 to about 5 mg/kg/day, from about 0.01 to about 4 mg/kg/day, from about 0.01 to about 3 mg/kg/day, from about 0.01 to about 2 mg/kg/day, from about 0.01 to about 1 mg/kg/day, or from about 0.01 to about 0.05 mg/kg/day.

The administered dose can also be expressed in units other than mg/kg/day. For example, doses for parenteral administration can be expressed as mg/m²/day. One of ordinary skill in the art would readily know how to convert doses from mg/kg/day to mg/m²/day to given either the height or weight of a subject or both (see, www.fda.gov/cder/cancer/animalframe.htm). For example, a dose of 1 mg/kg/day for a 65 kg human is approximately equal to 38 mg/m²/day.

In certain embodiments, the amount of the compound administered is sufficient to provide a plasma concentration of the compound at steady state, ranging from about 0.001 to about 500 μM, about 0.002 to about 200 μM, about 0.005 to about 100 μM, about 0.01 to about 50 μM, from about 1 to about 50 μM, about 0.02 to about 25 μM, from about 0.05 to about 20 μM, from about 0.1 to about 20 μM, from about 0.5 to about 20 μM, or from about 1 to about 20 μM.

In other embodiments, the amount of the compound administered is sufficient to provide a plasma concentration of the compound at steady state, ranging from about 5 to about 100 nM, about 5 to about 50 nM, about 10 to about 100 nM, about 10 to about 50 nM or from about 50 to about 100 nM.

As used herein, the term “plasma concentration at steady state” is the concentration reached after a period of administration of a compound provided herein, or a derivative thereof. Once steady state is reached, there are minor peaks and troughs on the time dependent curve of the plasma concentration of the compound.

In certain embodiments, the amount of the compound administered is sufficient to provide a maximum plasma concentration (peak concentration) of the compound, ranging from about 0.001 to about 50 μM, about 0.002 to about 200 μM, about 0.005 to about 100 μM, about 0.01 to about 50 μM, from about 1 to about 50 μM, about 0.02 to about 25 μM, from about 0.05 to about 20 μM, from about 0.1 to about 20 μM, from about 0.5 to about 20 μM, or from about 1 to about 20 μM.

In certain embodiments, the amount of the compound administered is sufficient to provide a minimum plasma concentration (trough concentration) of the compound, ranging from about 0.001 to about 500 μM, about 0.002 to about 200 μM, about 0.005 to about 100 μM, about 0.01 to about 50 μM, from about 1 to about 50 μM, about 0.01 to about 25 μM, from about 0.01 to about 20 μM, from about 0.02 to about 20 μM, from about 0.02 to about 20 μM, or from about 0.01 to about 20 μM.

In certain embodiments, the amount of the compound administered is sufficient to provide an area under the curve (AUC) of the compound, ranging from about 100 to about 100,000 ng*hr/mL, from about 1,000 to about 50,000 ng*hr/mL, from about 5,000 to about 25,000 ng*hr/mL, or from about 5,000 to about 10,000 ng*hr/mL.

The methods provided herein encompass treating a patient regardless of subject's age, although some diseases or disorders are more common in certain age groups.

Depending on the disease to be treated and the subject's condition, the compound provided herein, or a derivative thereof, may be administered by oral, parenteral (e.g., intramuscular, intraperitoneal, intravenous, CIV, intracisternal injection or infusion, subcutaneous injection, or implant), inhalation, nasal, vaginal, rectal, sublingual, or topical (e.g., transdermal or local) routes of administration. The compound provided herein, or a derivative thereof, may be formulated, alone or together, in suitable dosage unit with pharmaceutically acceptable excipients, carriers, adjuvants and vehicles, appropriate for each route of administration.

In one embodiment, the compound provided herein, or a derivative thereof, is administered orally. In another embodiment, the compound provided herein, or a derivative thereof, is administered parenterally. In yet another embodiment, the compound provided herein, or a derivative thereof, is administered intravenously.

The compound provided herein, or a derivative thereof, can be delivered as a single dose such as, e.g., a single bolus injection, or oral tablets or pills; or over time, such as, e.g., continuous infusion over time or divided bolus doses over time. The compound can be administered repeatedly if necessary, for example, until the subject experiences stable disease or regression, or until the subject experiences disease progression or unacceptable toxicity. For example, stable disease for solid tumors generally means that the perpendicular diameter of measurable lesions has not increased by 25% or more from the last measurement. Response Evaluation Criteria in Solid Tumors (RECIST) Guidelines, Journal of the National Cancer Institute 92(3): 205 216 (2000). Stable disease or lack thereof is determined by methods known in the art such as evaluation of patient symptoms, physical examination, visualization of the tumor that has been imaged using X-ray, CAT, PET, or MRI scan and other commonly accepted evaluation modalities.

The compound provided herein, or a derivative thereof, can be administered once daily (QD), or divided into multiple daily doses such as twice daily (BID), three times daily (TID), and four times daily (QID). In addition, the administration can be continuous (i.e., daily for consecutive days or every day), intermittent, e.g., in cycles (i.e., including days, weeks, or months of rest without drug). As used herein, the term “daily” is intended to mean that a therapeutic compound, such as the compound provided herein, or a derivative thereof, is administered once or more than once each day, for example, for a period of time. The term “continuous” is intended to mean that a therapeutic compound, such as the compound provided herein or a derivative thereof, is administered daily for an uninterrupted period of at least 10 days to 52 weeks. The term “intermittent” or “intermittently” as used herein is intended to mean stopping and starting at either regular or irregular intervals. For example, intermittent administration of the compound provided herein or a derivative thereof is administration for one to six days per week, administration in cycles (e.g., daily administration for two to eight consecutive weeks, then a rest period with no administration for up to one week), or administration on alternate days. The term “cycling” as used herein is intended to mean that a therapeutic compound, such as the compound provided herein or a derivative thereof, is administered daily or continuously but with a rest period. In some such embodiments, administration is once a day for two to six days, then a rest period with no administration for five to seven days.

In some embodiments, the frequency of administration is in the range of about a daily dose to about a monthly dose. In certain embodiments, administration is once a day, twice a day, three times a day, four times a day, once every other day, twice a week, once every week, once every two weeks, once every three weeks, or once every four weeks. In one embodiment, the compound provided herein, or a derivative thereof, is administered once a day. In another embodiment, the compound provided herein, or a derivative thereof, is administered twice a day. In yet another embodiment, the compound provided herein, or a derivative thereof, is administered three times a day. In still another embodiment, the compound provided herein, or a derivative thereof, is administered four times a day.

In certain embodiments, the compound provided herein, or a derivative thereof, is administered once per day from one day to six months, from one week to three months, from one week to four weeks, from one week to three weeks, or from one week to two weeks. In certain embodiments, the compound provided herein, or a derivative thereof, is administered once per day for one week, two weeks, three weeks, or four weeks. In one embodiment, the compound provided herein, or a derivative thereof, is administered once per day for 4 days. In one embodiment, the compound provided herein, or a derivative thereof, is administered once per day for 5 days. In one embodiment, the compound provided herein, or a derivative thereof, is administered once per day for 6 days. In one embodiment, the compound provided herein, or a derivative thereof, is administered once per day for one week. In another embodiment, the compound provided herein, or a derivative thereof, is administered once per day for two weeks. In yet another embodiment, the compound provided herein, or a derivative thereof, is administered once per day for three weeks. In still another embodiment, the compound provided herein, or a derivative thereof, is administered once per day for four weeks.

### VI. Methods of Treatment

Provided is a method of degrading CK1α in a cell by contacting the cell with a compound or composition provided herein. In another embodiment, provided is a method of degrading CK1a in a subject by administering to the subject a compound or composition provided herein.

In another embodiment, provided is a method of inhibiting Card11/BCL10/MALT1 (CBM) complex activation. It is known in the art that CK1a is required for TCR- and BCR-regulated activation of the CBM complex (see, e.g., Gehring et al., Cell Reports 2019, 29, 873-888; Bidere et al., Nature 2009, 458, 7234; Yin et al. Cell. Mol. Life Sci. 2022, 79, 112). Activation of the CBM complex leads to IL-2 induction, JNK signaling, and canonical NF-κB pathway signaling, and ultimately to cellular proliferation. Thus, degradation of CK1a leads to CBM complex inhibition and regulation of cellular proliferation.

Thus, in one embodiment, provided is a method of treating a subject having a proliferative disease by administering to the subject a compound or composition provided herein. In another embodiment, provided is a method of treating a subject having cancer by administering to the subject a compound or composition provided herein.

In one embodiment, the cancer is acute myeloid leukemia (AML), myelodysplastic syndrome, (MDS) (including 5q-MDS), colon cancer, acute lymphoblastic leukemia (ALL), chronic lymphocytic leukemia (CLL), chronic myeloid leukemia (CML), B-cell lymphoma or mantle cell lymphoma (MCL). See, e.g., Manni et al., Front. Oncol. 2021, 11, Article 733848; Tabe et al., Clin. Cancer Res. 2009, 15(3), 933-942; Liang et al., Mod. Pathol. 2010, 23(3), 389-91; Gehring et al., Cell Reports 2019, 29, 873-888; Bidere et al., Nature 2009, 458, 7234; Di Pilato et al., Nature 2019, 570(7759), 112-116; Rosenbaum et al., Nat. Commun. 2019, 10(1), 2352; Saba et al., Cancer Res. 2017, 77(24), 7038-7048.

In one embodiment, the cancer is a B-cell lymphoma. In another embodiment, the B-cell lymphoma is diffuse large B-cell lymphoma (DLBCL). In another embodiment, the DLBCL is ABC DLBCL.

In another embodiment, the cancer is a BTK inhibitor resistant cancer. In one embodiment, the BTK inhibitor resistant cancer is ibrutinib resistant cancer. In another embodiment, the ibrutinib resistant cancer is ABC DLBCL.

In another embodiment, the BTK inhibitor resistant cancer is acalabrutinib resistant cancer. In one embodiment, the BTK inhibitor resistant cancer is zanubrutinib resistant cancer. In one embodiment, the BTK inhibitor resistant cancer is resistant to one or more of pirtobrutinib, spebrutinib, evobrutinib, olmutinib, tirabrutinib, elsubrutinib (ABBV-105), tolebrutinib (SAR 442168), fenebrutinib, vacabrutinib, rilzabrutinib, M7583, BMS-986142, CT-1530, TG-1701, AC0058, SHR1459, RN-486, BIIB068 or DTRMWXHA-12.

In another embodiment, the BTK inhibitor resistant cancer is chronic lymphocytic leukemia (CLL), follicular lymphoma (FL), mantle cell lymphoma (MCL), marginal zone lymphoma (MZL), small lymphocytic lymphoma (SLL), Waldenstrom macroglobulinemia or chronic graft-versus-host disease.

In another embodiment, provided is a method of degrading CK1a and GSPT1 in a cell by contacting the cell with a compound or composition provided herein. In another embodiment, provided is a method of degrading CK1a and GSPT1 in a subject by administering to the subject a compound or composition provided herein. It is known in the art that loss of CK1a activity results in stabilization of p53 and inhibition of cell cycle progression. See, e.g., Huart et al., J. Biol. Chem. 2009, 284(47), 32384-32394. CK1a and MDM2 form a complex that regulates p53 and E2F-1 protein stability. The CK1α-MDM2 complex promotes degradation of p53, which in turn prevents expression of p53 targets, such as p21 (an inhibitor of cell cycle progression). GSPT1 degraders have shown efficacy in clinical trials against acute myeloid leukemia (AML). Degradation of CK1a (providing elevated p53), along with GSPT1 degradation leads to an improved therapeutic window and safety profile.

It is also known in the art that GSPT1 is implicated in a variety of cancers, including AIL, glioma, thyroid cancer, lung cancer, colorectal cancer, head and neck cancer, stomach cancer, liver cancer, pancreatic cancer, renal cancer, urothelial cancer, prostate cancer, testis cancer, breast cancer, cervical cancer, endometrial cancer, ovarian cancer, melanoma, multiple myeloma, hepatocellular carcinoma and gastric cancer. See, e.g., WO 2022/066835, WO 2022/029138, WO 2021/069705, WO 2018/169777, WO 2019/173224, WO 2019/241271, WO 2021/086830, WO 2022/007659, WO 2022/066835 and WO 2022/073469.

Thus, in another embodiment, provided is a method of treating AML in a subject by administering to the subject a compound or composition provided herein. In another embodiment, provided is a method of treating a solid tumor in a subject by administering to the subject a compound or composition provided herein. In another embodiment, the solid tumor is breast cancer. In another embodiment, provided is a method of treating glioma in a subject by administering to the subject a compound or composition provided herein. In another embodiment, provided is a method of treating thyroid cancer in a subject by administering to the subject a compound or composition provided herein. In another embodiment, provided is a method of treating lung cancer in a subject by administering to the subject a compound or composition provided herein. In another embodiment, provided is a method of treating colorectal cancer in a subject by administering to the subject a compound or composition provided herein. In another embodiment, provided is a method of treating head and neck cancer in a subject by administering to the subject a compound or composition provided herein. In another embodiment, provided is a method of treating stomach cancer in a subject by administering to the subject a compound or composition provided herein. In another embodiment, provided is a method of treating liver cancer in a subject by administering to the subject a compound or composition provided herein. In another embodiment, provided is a method of treating pancreatic cancer in a subject by administering to the subject a compound or composition provided herein. In another embodiment, provided is a method of treating renal cancer in a subject by administering to the subject a compound or composition provided herein. In another embodiment, provided is a method of treating urothelial cancer in a subject by administering to the subject a compound or composition provided herein. In another embodiment, provided is a method of treating prostate cancer in a subject by administering to the subject a compound or composition provided herein. In another embodiment, provided is a method of treating testis cancer in a subject by administering to the subject a compound or composition provided herein. In another embodiment, provided is a method of treating cervical cancer in a subject by administering to the subject a compound or composition provided herein. In another embodiment, provided is a method of treating endometrial cancer in a subject by administering to the subject a compound or composition provided herein. In another embodiment, provided is a method of treating ovarian cancer in a subject by administering to the subject a compound or composition provided herein. In another embodiment, provided is a method of treating melanoma in a subject by administering to the subject a compound or composition provided herein. In another embodiment, provided is a method of treating multiple myeloma in a subject by administering to the subject a compound or composition provided herein. In another embodiment, provided is a method of treating hepatocellular carcinoma in a subject by administering to the subject a compound or composition provided herein. In another embodiment, provided is a method of treating gastric cancer in a subject by administering to the subject a compound or composition provided herein.

As discussed above, CK1a is required for activation of Card11/BCL10/MALT1 (CBM) complex. It is known in the art that inhibition of MALT1 ameliorates autoimmune pathogenesis. See, e.g., Biswas et al., Frontiers in Immunology 2022, 13, 875320. Degradation of CK1α leads to inhibition of CBM activation and MALT1 signaling.

Thus, in another embodiment, provided is a method of treating a subject having an autoimmune disorder by administering to the subject a compound or composition provided herein. In one embodiment, the autoimmune disorder is Addison disease, Celiac disease—sprue (gluten-sensitive enteropathy), dermatomyositis, Graves' disease, Hashimoto thyroiditis, multiple sclerosis, myasthenia gravis, pernicious anemia, reactive arthritis, rheumatoid arthritis, Sjögren syndrome, systemic lupus erythematosus or type I diabetes.

It is known in the art that CK1α plays a role in promoting RAS-driven cancers, for example by destabilizing forkhead box O (FOXO) 3A/4 tumor suppressors, regulating oncogenic RAS-induced autophagy and phosphorylating Fas-associated death domain (FADD). See, e.g., Zhang et al. Oncogene, 2018, 37, 363-376; Cheong et al. J. Clin. Invest. 2015, 125(4), 1401-1418; Bowman et al. Sci. Signal. 2016, 8(361), ra9; Cheong et al. Mol. Cell. Onc. 2016, 3(3), e1045117. Thus, in one embodiment, provided herein is a method of treating a RAS-driven cancer in a subject by administering to the subject a compound or composition provided herein. In another embodiment, the RAS-driven cancer is a RAS-mutant cancer. In another embodiment, the RAS-driven cancer is a KRAS^{G12D}-driven cancer. In another embodiment, the RAS-driven cancer is lung cancer, head and neck cancer, pancreatic cancer, breast cancer, colorectal cancer, gastrointestinal cancer, melanoma, myeloid cancer, bladder cancer, cervical cancer, ovarian cancer or uterine cancer.

It is known in the art that inhibition of CK1α prevents acquired resistance to erlotinib in EGFR-mutant non-small cell lung cancer. See, e.g., Lantermann et al. Cancer Res. 2015, 75(22), 4937-4948. Thus, in one embodiment, provided herein is a method of preventing acquired resistance to erlotinib in EGFR-mutant non-small cell lung cancer in a subject by administering to the subject a compound or composition provided herein.

### VII. Combination Therapy with a Second Active Agent

The compound provided herein, or a derivative thereof, can also be combined or used in combination with other therapeutic agents useful in the treatment and/or prevention of proliferative diseases, including cancer and autoimmune disorders.

In one embodiment, provided herein is a method of treating, preventing, or managing a proliferative disease, comprising administering to a subject a compound provided herein, or a derivative thereof, in combination with one or more second active agents.

As used herein, the term “in combination” includes the use of more than one therapy (e.g., one or more prophylactic and/or therapeutic agents). However, the use of the term “in combination” does not restrict the order in which therapies (e.g., prophylactic and/or therapeutic agents) are administered to a subject with a disease or disorder. A first therapy (e.g., a prophylactic or therapeutic agent such as a compound provided herein, a compound provided herein, e.g., the compound provided herein, or a derivative thereof) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent) to the subject. Triple therapy is also contemplated herein.

Administration of the compound provided herein, or a derivative thereof and one or more second active agents to a subject can occur simultaneously or sequentially by the same or different routes of administration. The suitability of a particular route of administration employed for a particular active agent will depend on the active agent itself (e.g., whether it can be administered orally without decomposing prior to entering the blood stream) and the disease or disorder being treated.

The route of administration of the compound provided herein, or a derivative thereof, is independent of the route of administration of a second therapy. In one embodiment, the compound provided herein, or a derivative thereof, is administered orally. In another embodiment, the compound provided herein, or a derivative thereof, is administered intravenously. Thus, in accordance with these embodiments, the compound provided herein, or a derivative thereof, is administered orally or intravenously, and the second therapy can be administered orally, parenterally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, or in a slow release dosage form. In one embodiment, the compound provided herein, or a derivative thereof, and a second therapy are administered by the same mode of administration, orally or by IV. In another embodiment, the compound provided herein, or a derivative thereof, is administered by one mode of administration, e.g., by IV, whereas the second agent is administered by another mode of administration, e.g., orally.

In one embodiment, the second active agent is administered intravenously or subcutaneously and once or twice daily in an amount of from about 1 to about 1000 mg, from about 5 to about 500 mg, from about 10 to about 350 mg, or from about 50 to about 200 mg.

The specific amount of the second active agent will depend on the specific agent used, the type of disease being treated or managed, the severity and stage of disease, and the amount of the compound provided herein, or a derivative thereof, and any optional additional active agents concurrently administered to the subject.

One or more second active ingredients or agents can be used together with the compound provided herein, or a derivative thereof, in the methods and compositions provided herein. Second active agents can be large molecules (e.g., proteins) or small molecules (e.g., synthetic inorganic, organometallic, or organic molecules).

Examples of large molecule active agents include, but are not limited to, hematopoietic growth factors, cytokines, and monoclonal and polyclonal antibodies, particularly, therapeutic antibodies to cancer antigens. Typical large molecule active agents are biological molecules, such as naturally occurring or synthetic or recombinant proteins.

It is known in the art that targeting the CBM complex causes regulatory T-cells to prime tumors for immune checkpoint therapy. It is also known that MALT1 activity is key for regulatory T-cell immunosuppression. Thus, in one embodiment, the second active agent is a checkpoint inhibitor, such as an anti-CTLA-4, an anti-PD-1 or anti-PD-L1 antibody. In another embodiment, the second active agent is nivolumab, pembrolizumab, pidilizumab, atezolizumab, ipilimumab, tramelimumab, or a combination thereof. In these embodiments, the proliferative disease to be treated is cancer, including melanoma including unresectable or metastatic melanoma, BRAF 600 mutation positive, and melanoma with lymph node involvement; non-small cell lung cancer including metastatic non-small cell lung cancer; renal cell carcinoma; Hodgkin lymphoma including relapsed/refractory Hodgkin lymphoma; squamous cell carcinoma of the head and neck including metastatic disease; urothelial carcinoma including metastatic disease; colorectal cancer including metastatic disease; or hepatocellular carcinoma.

In one embodiment, the compound provided herein, or a derivative thereof, can be administered in an amount ranging from about 0.1 to about 150 mg, from about 1 to about 25 mg, or from about 2 to about 10 mg orally and daily alone, or in combination with a second active agent, prior to, during, or after the use of conventional therapy.

### VIII. Examples

The examples below are meant to illustrate certain embodiments provided herein, and not to limit the scope of this disclosure.

### Intermediate Example 1

### Synthesis of 3-(1-oxo-5-(4,4,5,5-tetramethyl-1,3,2-dioxaborolan-2-yl)isoindolin-2-yl)piperidine-2,6-dione

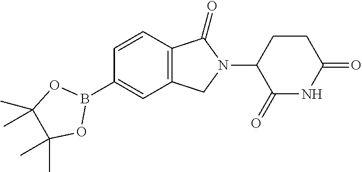 [SMILES: CC1(C)OB(c2ccc3c(c2)CN(C2CCC(=O)NC2=O)C3=O)OC1(C)C]

### Preparation of 3-(5-bromo-1-oxoisoindolin-2-yl)piperidine-2,6-dione

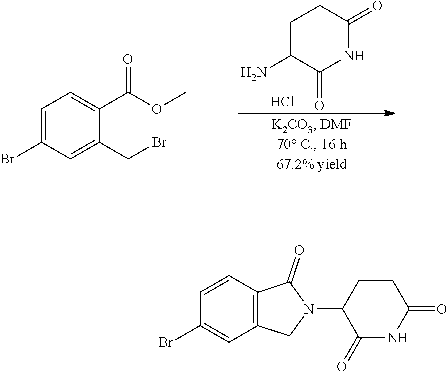 [SMILES: COC(=O)c1ccc(Br)cc1CBr ; NC1CCC(=O)NC1=O ; Cl ; O=C1CCC(N2Cc3cc(Br)ccc3C2=O)C(=O)N1]

To a solution of methyl 4-bromo-2-(bromomethyl)benzoate (20.0 g, 64.9 mmol, 1.00 eq) in DMF (170 mL) was added 3-aminopiperidine-2,6-dione (11.8 g, 71.4 mmol, 1.10 eq, HCl) and K₂CO₃ (26.9 g, 194 mmol, 3.00 eq). The resulting mixture was heated at 70° C. for 16 h. To the resulting residue, 500 mL water was added and the mixture stirred at 25° C. for 0.5 h. The resultant solid was filtered and washed with 200 ml ethyl acetate. The solid was dried under vacuum filtration to give the title compound (14.3 g, 43.6 mmol, 67.2% yield, 98.6% purity in HPLC at 220 nm) as a white solid. ¹H NMR: (400 MHz, DMSO-d₆) δ 10.49-11.15 (m, 8H), 7.89 (s, 1H), 7.66-7.73 (m, 2H), 5.10 (dd, J=18.4, 8.4 Hz, 1H), 4.32-4.49 (m, 2H), 2.61-2.89 (m, 1H), 2.56-2.60 (m, 1H), 2.38-2.55 (m, 1H), 2.00-2.36 (m, 1H). (ESI⁺) m/z: 322.7 (M+H)⁺, (C₁₃H₁₁BrN₂O₃).

### Preparation of 3-(1-oxo-5-(4,4,5,5-tetramethyl-1,3,2-dioxaborolan-2-yl)isoindolin-2-yl)piperidine-2,6-dione

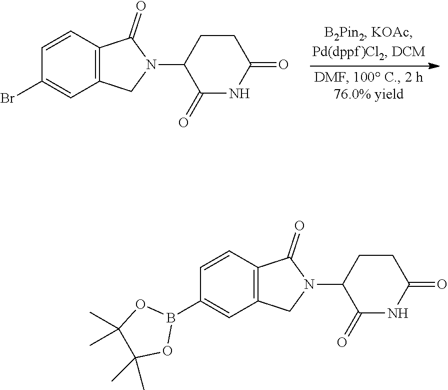 [SMILES: O=C1CCC(N2Cc3cc(Br)ccc3C2=O)C(=O)N1 ; CC1(C)OB(c2ccc3c(c2)CN(C2CCC(=O)NC2=O)C3=O)OC1(C)C]

To a solution of 3-(5-bromo-1-oxo-isoindolin-2-yl)piperidine-2,6-dione (10.0 g, 31.0 mmol, 1.00 eq) in DMF (60.0 mL) in a sealed tube was added bis(pinacolato)diboron (BPD) (8.64 g, 34.0 mmol, 1.10 eq), KOAc (9.11 g, 92.8 mmol, 3.00 eq), and Pd(dppf)Cl₂ (1.36 g, 1.86 mmol, 0.06 eq). The reaction mixture was heated at 100° C. for 2 h under N₂. 200 mL water was added to the reaction mixture and stirred at 25° C. for 0.25 h. The solid was precipitated, filtered, and dried under vacuum to give the title compound (8.90 g, 23.5 mmol, 76.0% yield, 97.8% purity in HPLC at 220 nm) as a white solid. ¹H NMR: (400 MHz, DMSO-d₆) δ 11.0 (s, 1H), 7.90 (s, 1H), 7.72-7.81 (m, 2H), 5.15 (dd, J=18.4, 8.4 Hz, 1H), 4.33-4.49 (m, 2H), 2.88-2.91 (m, 1H), 2.62-2.86 (m, 1H), 2.35-2.38 (m, 1H), 2.01-2.33 (m, 1H), 1.32 (s, 12H). (ESI⁺) m/z: 370.9 (M+H)⁺, (C₁₉H₂₃BN₂O₅).

### Example 1

### Synthesis of 3-(5-(1-Methyl-5-phenyl-1H-pyrazol-4-yl)-1-oxoisoindolin-2-yl)piperidine-2,6-dione

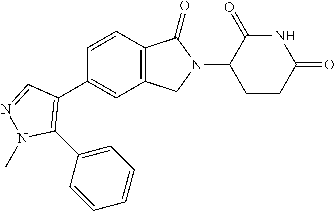 [SMILES: Cn1ncc(-c2ccc3c(c2)CN(C2CCC(=O)NC2=O)C3=O)c1-c1ccccc1]

A. 3-(5-(1-Methyl-5-phenyl-1H-pyrazol-4-yl)-1-oxoisoindolin-2-yl)piperidine-2,6-dione: To a solution of 3-(1-oxo-5-(4,4,5,5-tetramethyl-1,3,2-dioxaborolan-2-yl)isoindolin-2-yl)piperidine-2,6-dione (93.7 mg, 253 μmol, 1.20 eq), 4-bromo-1-methyl-5-phenyl-1H-pyrazole (50.0 mg, 211 μmol, 1.00 eq) and K₃PO₄ (89.5 mg, 422 μmol, 2.00 eq) in dioxane (1.25 mL) and H₂O (0.10 mL) was added (2-(2-aminophenyl)phenyl)-methylsulfonyloxy-palladium; dicyclohexyl-(2-(2,6-diisopropoxyphenyl)phenyl)phosphane (17.6 mg, 21.1 μmol, 0.10 eq) under at 25° C. The mixture was stirred at 100° C. for 1 h. The mixture was filtered through celite and the filter cake was washed with ethyl acetate (2×20.0 mL). The filtrate was concentrated under vacuum to give a residue at 40° C. The mixture was purified by preparative-HPLC (using a Welch Xtimate (150 mm×25 mm, 5 m) and gradient of 13-43% acetonitrile in water containing 0.05% HCl over 8 min at a flow rate of 25 mL/min to give the title compound (11.1 mg, 26.0 μmol, 12.3% yield) as a yellow solid. ¹H NMR: (400 MHz, DMSO-d₆) δ 10.96 (s, 1H), 7.90 (s, 1H), 7.55-7.52 (m, 4H), 7.52-7.51 (m, 3H), 7.39-7.22 (m, 1H), 5.06 (dd, J=5.2 Hz, J=13.6 Hz, 1H), 4.34 (d, J=17.6 Hz, 1H), 4.20 (d, J=17.2 Hz, 1H), 3.74 (s, 3H), 2.90-2.89 (m, 1H), 2.59-2.55 (m, 1H), 2.38-2.33 (m, 1H), 1.98-1.95 (m, 1H). (ESI⁺) m/z: 401.3 (M+H)⁺, (C₂₃H₂₀N₄O₃).

### Example 2

### Synthesis of 3-(5-(1-Methyl-5-phenyl-1H-imidazol-4-yl)-1-oxoisoindolin-2-yl)piperidine-2,6-dione

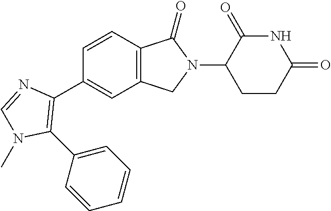 [SMILES: Cn1cnc(-c2ccc3c(c2)CN(C2CCC(=O)NC2=O)C3=O)c1-c1ccccc1]

A. 1-Methyl-5-phenyl-1H-imidazole: To a solution of methanamine hydrochloride (5.19 g, 76.8 mmol, 2.00 eq) in DMF (40.0 mL) was added benzaldehyde (4.08 g, 38.4 mmol, 3.89 mL, 1.00 eq) and DIEA (9.93 g, 76.8 mmol, 13.4 mL, 2.00 eq) at 25° C. The mixture was stirred at 25° C. for 2 h. Then K₂CO₃ (7.96 g, 57.63 mmol, 1.5 eq) and 1-((isocyanomethyl)-sulfonyl)-4-methylbenzene (9.00 g, 46.1 mmol, 1.20 eq) was added to the mixture at 25° C. The mixture was stirred at 50° C. for 32 h. The mixture was poured into water (50.0 mL) and brine (50.0 mL) extracted with ethyl acetate (3×100 mL) to collect the organic layer, the combined organic layers were dried over Na₂SO₄, filtered and the filtrate was concentrated under vacuum to get a residue. The residue was purified by column chromatography (SiO₂, Ethyl acetate:Petroleum ether=10/1 to Dichloromethane:Methanol=30/1) to give the title compound (2.20 g, 13.9 mmol, 36.1% yield, 99.7% purity in HPLC at 220 nm) as a yellow solid. ¹H NMR: (400 MHz, CDCl₃) δ 7.58 (s, 1H), 7.45-7.41 (m, 2H), 7.40-7.38 (m, 3H), 7.12 (s, 1H), 3.69 (s, 3H). (ESI⁺) m/z: 158.8 (M+H)⁺, (C₁₀H₁₀N₂).

B. 4-Bromo-1-methyl-5-phenyl-1H-imidazole: To a solution of 1-methyl-5-phenyl-1H-imidazole (530 mg, 3.34 mmol, 99.7% purity, 1.00 eq) in ACN (20.0 mL) was added NBS (624 mg, 3.51 mmol, 1.05 eq) at 20° C. The mixture was stirred at 20° C. for 1 h. The mixture was concentrated under reduce pressure to get a residue at 40° C. The residue was purified by column chromatography (SiO₂, Ethyl acetate:Petroleum ether=8/1 to 1/5). Then the residue was dissolved in DCM (20.0 mL) and washed with water (3×15.0 mL), the organic layer dried over Na₂SO₄, filtered and the filtrate was concentrated under vacuum to give the title compound (270 mg, 1.13 mmol, 33.8% yield, 99.5% purity in LCMS at 220 nm) as a yellow solid. ¹H NMR: (400 MHz, CDCl₃) δ 7.55 (s, 1H), 7.49-7.40 (m, 5H), 3.60 (s, 3H). (ESI⁺) m/z: 237.0 (M+H)⁺, (C₁₀H₉BrN₂).

C. 3-(5-(1-Methyl-5-phenyl-1H-imidazol-4-yl)-1-oxoisoindolin-2-yl)piperidine-2,6-dione: To a solution of 3-(1-oxo-5-(4,4,5,5-tetramethyl-1,3,2-dioxaborolan-2-yl)isoindolin-2-yl)piperidine-2,6-dione (224 mg, 604 μmol, 1.20 eq), 4-bromo-1-methyl-5-phenyl-1H-imidazole (120 mg, 504 μmol, 99.5% purity, 1.00 eq) and K₃PO₄ (214 mg, 1.01 mmol, 2.00 eq) in dioxane (3.00 mL) and H₂O (0.15 mL) was added [2-(2-aminophenyl)phenyl]-methylsulfonyloxy-palladium; dicyclohexyl-[2-(2,6-diisopropoxyphenyl)phenyl]phosphane (42.1 mg, 50.4 μmol, 0.10 eq) at 25° C. under N₂. The mixture was stirred at 100° C. for 1 h under N₂. The mixture was filtered through celite and the filter cake was washed with ethyl acetate (2×2.00 mL). The filtrate was concentrated under vacuum to give a residue at 40° C. The residue was purified by preparative-HPLC (using a Welch Ultimate C18 (150 mm×25 mm 5 μm) and gradient of 0-30% acetonitrile in water containing 0.1% FA over 10 min at a flow rate of 25 mL/min to give the title compound (40.4 mg, 99.3 μmol, 19.7% yield, 98.4% purity in HPLC at 220 nm) as an off-white solid. ¹H NMR: (400 MHz, DMSO-d₆) δ 10.96 (s, 1H), 7.85 (s, 1H), 7.61 (s, 1H), 7.52-7.44 (m, 4H), 7.42-7.40 (m, 3H), 5.06 (dd, J=4.8 Hz, J=13.2 Hz, 1H), 4.35 (d, J=17.2 Hz, 1H), 4.20 (d, J=17.6 Hz, 1H), 3.74 (s, 3H), 2.90-2.86 (m, 1H), 2.60-2.59 (m, 1H), 2.37-2.33 (m, 1H), 1.98-1.96 (m, 1H). (ESI⁺) m/z: 401.2 (M+H)⁺, (C₂₃H₂₀N₄O₃).

### Example 3

### Synthesis of 3-(5-(1-Methyl-3-phenyl-1H-pyrazol-4-yl)-1-oxoisoindolin-2-yl) piperidine-2, 6-dione

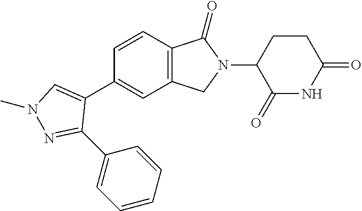 [SMILES: Cn1cc(-c2ccc3c(c2)CN(C2CCC(=O)NC2=O)C3=O)c(-c2ccccc2)n1]

A. 3-(5-(1-Methyl-3-phenyl-1H-pyrazol-4-yl)-1-oxoisoindolin-2-yl) piperidine-2, 6-dione: To a solution of 3-(5-bromo-1-oxoisoindolin-2-yl)piperidine-2,6-dione (100 mg, 309 μmol, 1.00 eq), 1-methyl-3-phenyl-4-(4,4,5,5-tetramethyl-1,3,2-dioxaborolan-2-yl)-1H-pyrazole (114 mg, 402 μmol, 1.30 eq) in DMF (1.00 mL) was added Pd(dppf)Cl₂ (50.5 mg, 61.9 μmol, 0.20 eq), K₃PO₄ (131 mg, 619 μmol, 2.00 eq) under N₂. The mixture was stirred at 90° C. for 12 h. The mixture was filtered to collect filtrate and was purified by prep-TLC (petroleum ether/ethyl acetate=0/1, R_{f}=0.3) to give the title compound (50.4 mg, 125 μmol, 20.0% yield, 98.9% purity in HPLC at 220 nm) as a yellow solid. ¹H NMR (400 MHz, DMSO-d₆): δ 10.98 (s, 1H), 8.05 (s, 1H), 7.65 (d, J=7.6 Hz, 1H), 7.46 (s, 1H), 7.40-7.32 (m, 6H), 5.13-5.08 (m, 1H), 4.43-4.38 (m, 2H), 3.93 (s, 1H), 2.94-2.87 (m, 2H), 2.60-2.54 (m, 1H), 2.45-2.38 (m, 1H). (ESI⁺) m/z: 401.0 (M+H)⁺, (C₂₃H₂₀N₄O₃).

### Example 4

### Synthesis of 3-(1-Oxo-5-(4-phenylthiophen-3-yl) isoindolin-2-yl)piperidine-2,6-dione

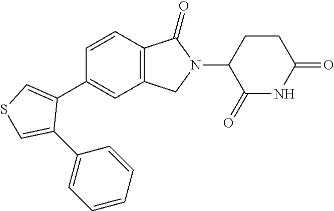 [SMILES: O=C1CCC(N2Cc3cc(-c4cscc4-c4ccccc4)ccc3C2=O)C(=O)N1]

Preparation of 3-(1-Oxo-5-(4-phenylthiophen-3-yl) isoindolin-2-yl) piperidine-2,6-dione: To a solution of 3-[1-oxo-5-(4,4,5,5-tetramethyl-1,3,2-dioxaborolan-2-yl)isoindolin-2-yl]piperidine-2,6-dione (158 mg, 418 μmol, 97.8% purity, 1.00 eq), 3-bromo-4-phenyl-thiophene (100 mg, 418.18 μmol, 1.00 eq), K₃PO₄ (176 mg, 836 μmol, 2.00 eq) in dioxane (3.00 mL) and H₂O (0.10 mL). Ru-Phos-Pd-G3 (70.0 mg, 83.6 μmol, 0.20 eq) was added to the reaction mixture under N₂. The mixture was stirred at 100° C. for 2 h under N₂. The mixture was filtered to collect filtrate and purified by preparative-HPLC (using a Phenomenex luna C18 (150 mm×25 mm 10 μm) and gradient of 38-68% acetonitrile in water containing 0.05% FA over 15 min at a flow rate of 25 mL/min to give the title compound (73.9 mg, 179 μmol, 42.8% yield, 97.3% purity in HPLC at 220 nm) as white solid. ¹H NMR (400 MHz, DMSO-d₆): δ 10.986 (s, 1H), 7.767-7.759 (d, J=3.2 Hz, 1H), 7.70-7.692 (d, J=3.2 Hz, 1H), 7.319-7.301 (m, 1H), 7.296 (s, 1H), 7.186-7.182 (m, 3H), 7.166-7.163 (m, 3H), 5.067-4.98 (m, 1H), 4.425-4.382 (m, 1H), 4.294-4.240 (m, 1H), 2.944-2.880 (m, 1H), 2.619-2.608 (m, 1H), 2.413-2.381 (m, 1H), 2.022-2.005 (m, 1H). (ESI⁺) m/z: 403.0 (M+H)⁺, (C₂₃H₁₈N₂O₃S).

### Example 5

### Synthesis of 3-(1-Oxo-5-(3-phenyl-1H-pyrazol-4-yl) isoindolin-2-yl) piperidine-2, 6-dione

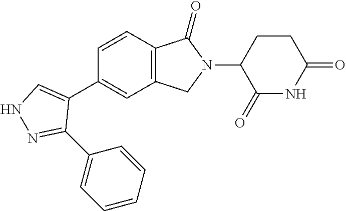 [SMILES: O=C1CCC(N2Cc3cc(-c4c[nH]nc4-c4ccccc4)ccc3C2=O)C(=O)N1]

A. 3-(1-Oxo-5-(3-phenyl-1H-pyrazol-4-yl) isoindolin-2-yl) piperidine-2,6-dione: To a solution of 4-bromo-3-phenyl-1H-pyrazole (100 mg, 448 μmol, 1.00 eq), 3-[1-oxo-5-(4,4,5,5-tetramethyl-1,3,2-dioxaborolan-2-yl)isoindolin-2-yl]piperidine-2,6-dione (174 mg, 471 μmol, 1.05 eq), (A-taphos)₂PdCl₂ (63.5 mg, 90.0 μmol, 63.5 μL, 0.20 eq) in dioxane (4.00 mL) and H₂O (0.40 mL) was added K₃PO₄ (190 mg, 897 μmol, 2.00 eq) under N₂. The mixture was stirred at 100° C. for 12 h under N₂. The mixture was filtered to collect filtrate and purified by preparative-HPLC (using a Phenomenex luna C18 (150 mm×25 mm 10 μm) and gradient of 15-45% acetonitrile in water containing 0.05% FA over 18 min at a flow rate of 25 mL/min to give the title compound (5.91 mg, 15.2 μmol, 3.39% yield, 99.4% purity in HPLC at 220 nm) as a yellow solid. ¹H NMR (400 MHz, DMSO-d₆): δ 11.0 (s, 1H), 7.503-7.484 (d, J=7.6 Hz, 1H), 7.50 (s, 1H), 7.412-7.278 (m, 7H), 5.12-5.080 (m, 1H), 4.427-4.248 (m, 2H), 2.943-2.878 (m, 1H), 2.617-2.612 (m, 1H), 2.410-2.381 (m, 1H), 2.010-1.984 (m, 2H). (ESI⁺) m/z: 387.0 (M+H)⁺, (C₂₂H₁₈N₄O₃).

### Example 6

### Synthesis of 3-(1-oxo-5-(1-phenyl-1H-pyrazol-5-yl)isoindolin-2-yl)piperidine-2,6-dione

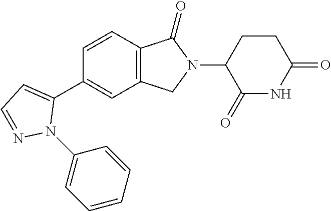 [SMILES: O=C1CCC(N2Cc3cc(-c4ccnn4-c4ccccc4)ccc3C2=O)C(=O)N1]

A. 3-(1-Oxo-5 (1-phenyl-1H-pyrazol-5-yl)isoindolin-2-yl)piperidine-2,6-dione: To a solution of 3-(5-bromo-1-oxo-isoindolin-2-yl)piperidine-2,6-dione (50.0 mg, 154 μmol, 1.00 eq), 1-phenyl-5-(4,4,5,5-tetramethyl-1,3,2-dioxaborolan-2-yl)-1H-pyrazole (54.3 mg, 201 μmol, 1.30 eq) in dioxane (1.00 mL) and H₂O (0.05 mL) under N₂, then added (A-taphos)₂PdCl₂ (25.8 mg, 30.9 μmol, 0.20 eq), K₃PO₄ (65.6 mg, 309 μmol, 2.00 eq) under N₂. The reaction mixture was stirred at 100° C. for 3 h under N₂. The reaction mixture was concentrated in vacuum and purified by preparative-HPLC (using a Phenomenex Luna C18 (150 mm×25 mm 10 μm) and gradient of 22-42% acetonitrile in water containing 0.05% FA over 63 mins at a flow rate of 25 mL/min to give the title compound (15.0 mg, 38.5 μmol, 24.8% yield, 99.2% purity in HPLC at 220 nm) as a white solid. ¹H NMR: (400 MHz, DMSO-d₆) δ 10.9 (s, 1H), 7.80 (d, J=1.6 Hz, 1H), 7.67 (d, J=7.6 Hz, 1H), 7.55 (s, 1H), 7.37-7.46 (m, 3H), 7.26-7.30 (m, 3H), 6.76 (d, J=2.0 Hz, 1H), 5.11 (dd, J=13.6 Hz, 5.2 Hz, 1H), 4.26-4.44 (m, 2H), 2.85-2.95 (m, 1H), 2.56-2.61 (m, 1H), 2.36-2.44 (m, 1H), 1.96-2.03 (m, 1H). (ESI⁺) m/z: 387.2 (M+H)⁺, (C₂₂H₁₈N₄O₃).

### Example 7

### Synthesis of 3-(1-Oxo-5-(1-phenyl-1H-imidazol-2-yl)isoindolin-2-yl)piperidine-2,6-dione

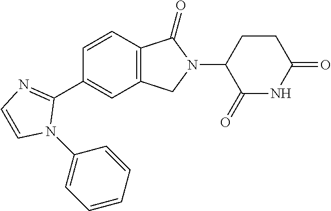 [SMILES: O=C1CCC(N2Cc3cc(-c4nccn4-c4ccccc4)ccc3C2=O)C(=O)N1]

A. 2-Iodo-1-phenyl-1H-imidazole: To a solution of 1-phenylimidazole (1.90 g, 13.1 mmol, 1.00 eq) in THF (95.0 mL) was cooled to −65˜−60° C. n-BuLi (2.50 M, 7.04 mL, 1.33 eq) was added dropwise under N₂ at −65˜−60° C. Then the mixture was stirred at −60° C. for 1.5 h. A solution of iodine (10.0 g, 39.5 mmol, 7.96 mL, 3.00 eq) in THF (100 mL) was added to the mixture at −65˜−60° C. The reaction mixture was stirred at 25° C. for 0.5 h. The reaction mixture was poured into saturated aqueous NH₄Cl (200 mL) and extracted with EtOAc (3×200 mL) to collected the organic layer. The organic layer were washed with saturated aqueous Na₂S₂O₃ solution (2×100 mL), dried over anhydrous Na₂SO₄, filter and the filtrate is concentrated under reduced pressure to get a residue. The residue was purified by column chromatography (SiO₂, Petroleum ether/Ethyl acetate=20/1 to 5/1, TLC: Petroleum ether/Ethyl acetate=5/1, R_{f}=0.50) to give the title compound (2.20 g, 8.15 mmol, 61.8% yield) was obtained as a yellow solid. ¹H NMR: (400 MHz, CDCl₃) δ 7.52-7.49 (m, 3H), 7.36-7.34 (m, 2H), 7.21 (s, 2H). (ESI⁺) m/z: 271.1 (M+H)⁺, (C₉H₇IN₂).

B. 3-(1-Oxo-5-(1-phenyl-1H-imidazol-2-yl)isoindolin-2-yl)piperidine-2,6-dione: To a solution of 2-iodo-1-phenyl-imidazole (100 mg, 370. μmol, 1.00 eq) and 3-[1-oxo-5-(4,4,5,5-tetramethyl-1,3,2-dioxaborolan-2-yl)isoindolin-2-yl]piperidine-2,6-dione (137 mg, 370 μmol, 1.00 eq) in dioxane (3.00 mL) and H₂O (0.15 mL) was added (A-taphos)₂PdCl₂ (61.9 mg, 74.0 μmol, 0.20 eq) and K₃PO₄ (157 mg, 740 μmol, 2.00 eq) under N₂. The mixture was stirred at 100° C. for 1 h. The mixture was poured into H₂O (20.0 mL) and extracted with EtOAc (3×20.0 mL) to collected the organic layers. The organic layers were dried over anhydrous Na₂SO₄, filtered and the filtrate was concentrated under reduced pressure to get a residue. The residue was purified by preparative-HPLC (using a Phenomenex luna C18 (150×25 mm×10 μm) and gradient of 42.0%-72.0% acetonitrile in water containing 0.05% FA over 13 min at a flow rate of 25 mL/min) to give the title compound (6.84 mg, 17.3 μmol, 4.68% yield, 97.8% purity in HPLC at 220 nm) was obtained as white solid. ¹H NMR: (400 MHz, DMSO-d₆) δ 10.9 (s, 1H), 7.60 (d, J=6.0 Hz, 2H), 7.57 (d, J=1.2 Hz, 1H), 7.52-7.44 (m, 3H), 7.35-7.31 (m, 3H), 7.25 (d, J=1.2 Hz, 1H), 5.09 (dd, J=13.2 Hz, J=5.2 Hz, 1H), 4.43-4.38 (m, 1H), 4.28-4.24 (m, 1H), 2.96-2.85 (m, 1H), 2.60 (br, 1H), 2.40-2.34 (m, 1H), 2.01-1.96 (m, 1H). (ESI⁺) m/z: 387.1 (M+H)⁺, (C₂₂H₁₈N₄O₃).

### Example 8

### Synthesis of 3-(5-(5-(4-fluorophenyl)-1-methyl-1H-pyrazol-4-yl)-1-oxoisoindolin-2-yl)piperidine-2,6-dione

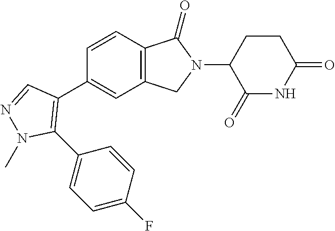 [SMILES: Cn1ncc(-c2ccc3c(c2)CN(C2CCC(=O)NC2=O)C3=O)c1-c1ccc(F)cc1]

A. 3-(5-(5-(4-Fluorophenyl)-1-methyl-1H-pyrazol-4-yl)-1-oxoisoindolin-2-yl)piperidine-2,6-dione: To a solution of 3-[5-(5-bromo-1-methyl-pyrazol-4-yl)-1-oxo-isoindolin-2-yl]piperidine-2,6-dione (39.8 mg, 90.8 μmol, 91.9% purity, 1.00 eq) in dioxane (1.00 mL) was added 2-(4-fluorophenyl)-4,4,5,5-tetramethyl-1,3,2-dioxaborolane (201 mg, 908 μmol, 10.0 eq), K₂CO₃ (50.2 mg, 363 μmol, 4.00 eq) and Pd(PPh₃)₄ (21.0 mg, 18.1 μmol, 0.200 eq). The reaction mixture was stirred at 100° C. for 4 h under N₂. After the reaction was completed, the reaction mixture was filtered through diatomite and the filtrate was concentrated under vacuum to give a residue. The residue was purified by preparative-HPLC (using a Welch Xtimate (C18 150×25 mm×5 μm) and gradient of 15-45% acetonitrile in water containing 0.05% HCl over 15 min at a flow rate of 25 mL/min to give the title compound (5.65 mg, 13.5 μmol, 14.8% yield, >99% purity in HPLC at 220 nm) as a white solid. ¹H NMR: (400 MHz, DMSO-d₆) δ 11.0 (s, 1H), 7.90 (s, 1H), 7.60-7.55 (m, 1H), 7.49-7.43 (m, 2H), 7.40-7.33 (m, 3H), 7.25-7.20 (m, 1H), 5.08 (dd, J=13.6, 5.2 Hz, 1H), 4.36 (d, J=16.8 Hz, 1H), 4.22 (d, J=17.2 Hz, 1H), 3.72 (s, 3H), 2.95-2.84 (m, 1H), 2.63-2.59 (m, 1H), 2.43-2.31 (m, 1H), 2.01-1.94 (m, 1H). (ESI⁺) m/z: 403.0 (M+H)⁺, (C₂₃H₁₉FN₄O₃).

### Example 9

### Synthesis of 3-(1-Oxo-5-(1-phenyl-1H-pyrazol-4-yl)isoindolin-2-yl)piperidine-2,6-dione

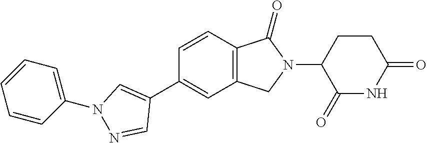 [SMILES: O=C1CCC(N2Cc3cc(-c4cnn(-c5ccccc5)c4)ccc3C2=O)C(=O)N1]

A. 3-(1-Oxo-5-(1-phenyl-1H-pyrazol-4-yl)isoindolin-2-yl)piperidine-2,6-dione: To a solution of 3-(1-oxo-5-(4,4,5,5-tetramethyl-1,3,2-dioxaborolan-2-yl)isoindolin-2-yl)piperidine-2,6-dione (100 mg, 270 μmol, 1.00 eq) and 4-bromo-1-phenyl-pyrazole (72.3 mg, 324 μmol, 1.20 eq) in dioxane (2.00 mL) and H₂O (0.10 mL) was added K₃PO₄ (114 mg, 540 μmol, 2.00 eq) and Ru-Phos-Pd-G₃ (22.6 mg, 27.0 μmol, 0.10 eq). The reaction mixture was stirred at 100° C. for 2 h under N₂. Then the reaction mixture was filtered through diatomite and the filtrate was concentrated under vacuum to give a crude product. The crude product was purified by preparative TLC (SiO₂, Dichloromethane:Methanol=10:1, R_{f}=0.40) and preparative HPLC (using a Welch Xtimate Cis 150 mm×25 mm×5 μm) and gradient of 21%-51% acetonitrile in water containing HCl (0.10 mol/L, 9 min) to give the title compound (15.2 mg, 39.1 μmol, 14.4% yield, 99.4% purity in HPLC at 220 nm) as a white solid. ¹H NMR: (400 MHz, DMSO-d₆) δ 11.00 (s, 1H), 9.17 (s, 1H), 8.35 (s, 1H), 7.96 (s, 1H), 7.92-7.89 (m, 3H), 7.74 (d, J=8.0 Hz, 1H), 7.58 (t, J=8.4 Hz, 2H), 7.36 (t, J=7.2 Hz, 1H), 5.15-5.10 (m, J=5.2 Hz, 1H), 4.53 (d, J=17.2 Hz, 1H), 4.39 (d, J=17.2 Hz, 1H), 2.97-2.88 (m, 1H), 2.63 (d, J=17.2 Hz, 1H), 2.46-2.42 (m, 1H), 2.04-2.00 (m, 1H). (ESI⁺) m/z: 387.1 (M+H)⁺, (C₂₂H₁₈N₄O₃).

### Example 10

### Synthesis of 3-(5-(5-(4-Fluorophenyl)-1-methyl-1H-imidazol-4-yl)-1-oxoisoindolin-2-yl)piperidine-2,6-dione

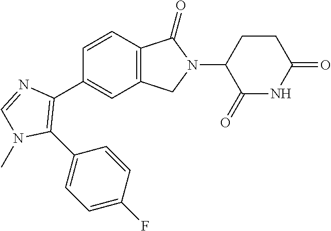 [SMILES: Cn1cnc(-c2ccc3c(c2)CN(C2CCC(=O)NC2=O)C3=O)c1-c1ccc(F)cc1]

A. 3-(5-(1-Methyl-1H-imidazol-4-yl)-1-oxoisoindolin-2-yl)piperidine-2,6-dione: A microwave vial was charged with 3-(5-bromo-1-oxoisoindolin-2-yl)piperidine-2,6-dione (400 mg, 1.24 mmol, 1.00 eq), 1-methyl-4-(tributylstannyl)-1H-imidazole (597 mg, 1.61 mmol, 1.30 eq) in DMF (4.00 mL) under N₂, and degassed with N₂ for 10 min, Pd(dppf)Cl₂·CH₂Cl₂ (202 mg, 247 μmol, 0.20 eq) was added to the mixture. The reaction mixture was stirred at 130° C. for 1.5 h in microwave under N₂. The mixture was filtered to collect liquid and the liquid was concentrated under reduced pressure to get residue. The residue was purified by preparative-TLC (Dichloromethane/Methanol=10/1, R_{f}=0.30) to get title compound (60.0 mg, 181 μmol, 7.30% yield, 98.0% purity in LCMS at 220 nm) as brown solid. (ESI⁺) m/z: 324.0 (M+H)⁺, (C₁₇H₁₆N₄O₃).

B. 3-(5-(5-Bromo-1-methyl-1H-imidazol-4-yl)-1-oxoisoindolin-2-yl)piperidine-2,6-dione: To a solution of 3-(5-(1-methyl-1H-imidazol-4-yl)-1-oxoisoindolin-2-yl)piperidine-2,6-dione (300 mg, 1.51 mmol, 1.00 eq) in MeOH (3.00 mL) was added NBS (296 mg, 1.66 mmol, 1.10 eq) at 0° C. The mixture was stirred at 25° C. for 3 h. Then the reaction mixture was concentrated under reduced pressure to get residue. The residue was purified by preparative-TLC (Dichloromethane:Methanol=10:1, R_{f}=0.32) to get title compound (40.0 mg, 98.9 μmol, 50.1% yield, 99.7% purity in LCMS at 220 nm) as brown solid. (ESI⁺) m/z: 403.2 (M+H)⁺, (C₁₇H₁₅BrN₄O₃).

C. 3-(5-(5-(4-Fluorophenyl)-1-methyl-1H-imidazol-4-yl)-1-oxoisoindolin-2-yl)piperidine-2,6-dione: To a solution of 3-(5-(5-bromo-1-methyl-1H-imidazol-4-yl)-1-oxoisoindolin-2-yl)piperidine-2,6-dione (39.2 mg, 96.9 μmol, 99.7% purity, 1.00 eq), 2-(4-fluorophenyl)-4,4,5,5-tetramethyl-1,3,2-dioxaborolane (107 mg, 484 μmol, 5.00 eq) in dioxane (3.00 mL) and H₂O (0.15 mL) was added K₂CO₃ (40.2 mg, 290 μmol, 3.00 eq) and Pd(dppf)Cl₂·CH₂Cl₂ (15.8 mg, 19.4 μmol, 0.20 eq) under N₂. The mixture was stirred at 100° C. for 2 h under N₂. The mixture was filtered to collect filtered liquid, and the liquid was concentrated under reduced pressure to get residue. The residue was purified by preparative-HPLC (using a Phenomenex luna C18 (150×25 mm×5 μm) and gradient of 3.00%-33.0% acetonitrile in water containing 0.05% FA over 15 min at a flow rate of 25 mL/min) to give the title compound (1.28 mg, 2.93 μmol, 3.00% yield, 95.7% purity in HPLC at 220 nm) as white solid. ¹H NMR: (400 MHz, DMSO-d₆) δ 10.9 (s, 1H), 7.59 (s, 1H), 7.55-7.53 (m, 1H), 7.48-4.47 (m, 1H), 7.46-7.44 (m, 3H), 7.38-7.35 (m, 2H), 5.15-5.01 (m, 1H), 4.38-4.34 (m, 1H), 4.32-4.16 (m, 1H), 3.47 (s, 3H), 2.90-2.85 (m, 1H), 2.62-2.57 (m, 1H), 2.37-2.36 (m, 1H), 2.34-2.32 (m, 1H). (ESI⁺) m/z: 419.3 (M+H)⁺, (C₂₃H₁₉FN₄O₃).

### Example 11

### Synthesis of (3-(5-(5-Methyl-4-phenyl-1H-pyrazol-3-yl)-1-oxoisoindolin-2-yl)piperidine-2,6-dione

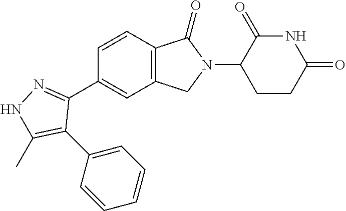 [SMILES: Cc1[nH]nc(-c2ccc3c(c2)CN(C2CCC(=O)NC2=O)C3=O)c1-c1ccccc1]

A. 3-Iodo-5-methyl-4-phenyl-1H-pyrazole: To a solution of 5-methyl-4-phenyl-1H-pyrazole (100 mg, 632 μmol, 1.00 eq) in THF (0.80 mL) and H₂O (0.80 mL) was added NaI (189 mg, 1.26 mmol, 2.00 eq), 12 (802 mg, 3.16 mmol, 637 μL, 5.00 eq) and K₂CO₃ (174 mg, 1.26 mmol, 2.00 eq). The mixture was stirred at 100° C. for 48 h. The combined mixture was quenched with quenched with Na₂SO₃ (10.0%, 35.0 mL). The mixture was extracted with ethyl acetate (3×15.0 mL), dried over Na₂SO₄, filtered to collect filtrated liquid and concentrated under reduced pressure to get residue. Then the residue was purified by preparative-TLC (Petroleum ether/Ethyl acetate=3/1, R_{f}=0.30) to give the title compound (114 mg, 399 μmol, 63.2% yield, 99.6% purity in LCMS at 220 nm) as white solid. ¹H NMR: (400 MHz, DMSO-d₆) δ 13.1 (s, 1H), 7.44-7.43 (m, 2H), 7.42-7.40 (m, 3H), 2.29-2.24 (m, 3H). (ESI⁺) m/z: 284.1 (M+H)⁺, (C₁₀H₉IN₂).

B. 3-(5-(5-Methyl-4-phenyl-1H-pyrazol-3-yl)-1-oxoisoindolin-2-yl)piperidine-2,6-dione: To a solution of 3-iodo-5-methyl-4-phenyl-1H-pyrazole (80.0 mg, 281 μmol, 1.00 eq), 3-(1-oxo-5-(4,4,5,5-tetramethyl-1,3,2-dioxaborolan-2-yl)isoindolin-2-yl)piperidine-2,6-dione (114 mg, 309. μmol, 1.10 eq) and K₃PO₄ (119 mg, 563 μmol, 2.00 eq) in dioxane (1.00 mL) and H₂O (0.05 mL) was added Ru-Phos-Pd-G₃ (47.1 mg, 56.3 μmol, 0.20 eq) under N₂. The mixture was stirred at 100° C. under N₂ for 60 h. Then the reaction mixture was filtered to get filtered liquid, and the liquid was concentrated under reduced pressure to get residue. The residue was purified by preparative-HPLC (using a Phenomenex luna C18 (150×25 mm×5 μm) and gradient of 29.0%-49.0% acetonitrile in water containing 0.05% FA over 58 mins at a flow rate of 25 mL/min to give the title compound (3.97 mg, 9.71 μmol, 3.45% yield, 97.9% purity in HPLC at 220 nm) was obtained as white solid. ¹H NMR: (400 MHz, DMSO-d₆) δ 11.0 (s, 1H), 8.36 (s, 1H), 7.70 (s, 2H), 7.44-7.39 (m, 3H), 7.37-7.35 (m, 1H), 7.31-7.27 (m, 2H), 5.13-5.06 (m, 1H), 4.28-4.24 (m, 1H), 4.23-4.20 (m, 1H), 2.93-2.63 (m, 1H), 2.61-2.60 (m, 1H), 2.39-2.32 (m, 1H), 2.22 (s, 3H), 2.00-1.97 (m, 1H), (ESI⁺) m/z: 400.0 (M+H)⁺, (C₂₃H₂₀N₄O₃).

### Example 12

### Synthesis of 3-(5-(2-cyclopropyl-1-methyl-5-phenyl-1H-imidazol-4-yl)-1-oxoisoindolin-2-yl)piperidine-2,6-dione

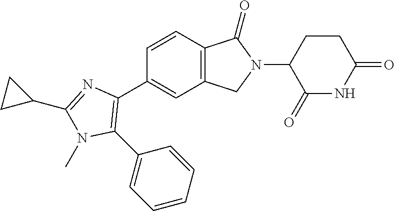 [SMILES: Cn1c(C2CC2)nc(-c2ccc3c(c2)CN(C2CCC(=O)NC2=O)C3=O)c1-c1ccccc1]

A. N-(2, 2-Dimethoxyethyl)-N-methylcyclopropanecarboximidamide: To a solution of 2,2-dimethoxy-N-methylethan-1-amine (3.00 g, 25.2 mmol, 3.24 mL, 1.00 eq) and cyclopropanecarbonitrile (2.11 g, 31.5 mmol, 2.32 mL, 1.25 eq) was added CuCl (3.12 g, 31.5 mmol, 752 μL, 1.25 eq). The mixture was stirred at 85° C. for 12 h. Concentrated reaction mixture under reduced pressure to give the title compound (5.00 g, crude) as yellow oil. (ESI⁺) m/z: 186 (C₉H₁₈N₂₀₂)

B. 2-Cyclopropyl-1-methyl-1H-imidazole: To a solution of N-(2, 2-dimethoxyethyl)-N-methylcyclopropanecarboximidamide (5.00 g, 26.9 mmol, 1.00 eq) in MeOH (15.0 mL) was added con HCl (12.0 M, 2.24 mL, 1.00 eq). The mixture was stirred at 80° C. for 4 h. Concentrated reaction mixture under reduced pressure to get residue. The residue was triturated with MTBE (20.0 mL) at 20° C. for 30 min to give the title compound (2.00 g, 16.4 μmol, 60.9% yield) as yellow oil. ¹H NMR: (400 MHz, CDCl₃) δ 7.27 (d, J=8.0 Hz, 1H), 7.01 (d, J=15.6 Hz, 1H), 3.88 (s, 3H), 1.99-1.96 (m, 1H), 1.20-1.14 (m, 4H). (ESI⁺) m/z: 123.1 (M+H)⁺, (C₇H₁₀N₂).

C. 2-Cyclopropyl-1-methyl-5-phenyl-1H-imidazole: To a solution of 2-cyclopropyl-1-methyl-1H-imidazole (1.00 g, 8.19 mmol, 1.00 eq), PCy₃ (229 mg, 819 μmol, 265 μL, 0.10 eq), NaOtBu (2.36 g, 24.5 mmol, 3.00 eq) in o-xylene (40.0 mL) was added chlorobenzene (2.76 g, 24.5 mmol, 2.49 mL, 3.00 eq), Pd(OAc)₂ (91.9 mg, 409 μmol, 0.05 eq) under N₂. The mixture was stirred at 130° C. for 17 h under N₂. Then the mixture was diluted with petroleum ether (100 mL), and the organic layer was filtered and concentrated under reduced pressure to get residue. The residue was purified by column chromatography (SiO₂, Petroleum ether/Ethyl acetate=10/1 to 2/1, R_{f}=0.20) to get the title compound (1.33 g, 6.35 mmol, 77.5% yield, 95.0% purity in LCMS at 220 nm) as yellow oil. (ESI⁺) m/z: 199.0 (M+H)⁺, (C₁₃H₁₄N₂).

D. 4-Bromo-2-cyclopropyl-1-methyl-5-phenyl-1H-imidazole: To a solution of 2-cyclopropyl-1-methyl-5-phenyl-1H-imidazole (300 mg, 1.51 mmol, 1.00 eq) in MeOH (3.00 mL) was added NBS (296 mg, 1.66 mmol, 1.10 eq) at 0° C. The mixture was stirred at 25° C. for 3 h. Then the reaction mixture was concentrated under reduced pressure to get residue. The residue was purified by preparative-TLC (Petroleum ether:Ethyl acetate=3:1, R_{f}=0.30) to get title compound (380 mg, 1.05 mmol, 69.7% yield, 76.9% purity in LCMS at 220 nm) as yellow oil. (ESI⁺) m/z: 277.0 (M+H)⁺, (C₁₃H₁₃BrN₂).

E. 3-(5-(2-Cyclopropyl-1-methyl-5-phenyl-1H-imidazol-4-yl)-1-oxoisoindolin-2-yl) piperidine-2, 6-dione: To a solution of 3-(1-oxo-5-(4,4,5,5-tetramethyl-1,3,2-dioxaborolan-2-yl)isoindolin-2-yl)piperidine-2,6-dione (701 mg, 1.89 mmol, 1.50 eq), 4-bromo-2-cyclopropyl-1-methyl-5-phenyl-1H-imidazole (350 mg, 1.26 mmol, 1.00 eq) and K₃PO₄ (536 mg, 2.53 mmol, 2.00 eq) in dioxane (3.00 mL) and H₂O (0.15 mL) was added Ru-Phos-Pd-G₃ (211 mg, 253 μmol, 0.20 eq) under N₂. The mixture was stirred at 100° C. for 2 h under N₂. The mixture was filtered to collect filtered liquid, and the liquid was concentrated under reduced pressure to get residue. The residue was purified by preparative-HPLC (using a Phenomenex luna C18 (150×25 mm×5 μm) and gradient of 2.00%-32.0% acetonitrile in water containing 0.05% FA over 15 min at a flow rate of 25 mL/min to give the title compound (33.1 mg, 72.2 μmol, 5.70% yield, 96.2% purity in HPLC at 220 nm) as white solid. ¹H NMR: (400 MHz, DMSO-d₆) δ 10.9 (s, 1H), 7.57-7.46 (m, 5H), 7.39-7.36 (m, 3H), 4.99-4.98 (m, 1H), 4.35-4.22 (m, 1H), 4.20-4.13 (m, 1H), 3.46 (s, 3H), 2.88-2.85 (m, 1H), 2.60-2.59 (m, 1H), 2.36-2.33 (m, 1H), 2.08-2.07 (m, 1H), 2.05-1.97 (m, 1H), 1.01-0.97 (m, 4H). (ESI⁺) m/z: 441.0 (M+H)⁺, (C₂₆H₂₄N₄O₃).

### Example 13

### Synthesis of 3-(1-Oxo-5-(5-phenyl-1H-imidazol-4-yl) isoindolin-2-yl) piperidine-2,6-dione

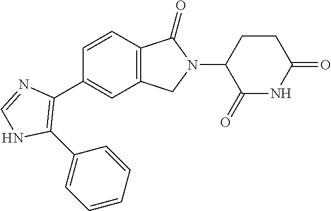 [SMILES: O=C1CCC(N2Cc3cc(-c4nc[nH]c4-c4ccccc4)ccc3C2=O)C(=O)N1]

A. 4-Bromo-5-phenyl-1H-imidazole: To a solution of 5-phenyl-1H-imidazole (200 mg, 1.39 mmol, 1.00 eq) in MeOH (3.00 mL) was added NBS (259 mg, 1.46 mmol, 1.05 eq). The mixture was stirred at 0° C. for 1 h. The reaction mixture was poured into H₂O (15.0 mL) and extracted with Ethyl acetate. The combined organic layer was washed with brine (3×10.0 mL), dried over Na₂SO₄, filtered and concentrated to give the title compound (98.0 mg, 431 μmol, 31.1% yield, 98.3% purity in LCMS at 220 nm) was obtained as light yellow solid. ¹H NMR: (400 MHz, CDCl₃) (12.9-12.8 (m, 1H), 7.77-7.75 (m, 1H), 7.72-7.70 (m, 2H), 7.49-7.45 (m, 2H), 7.63-7.33 (m, 1H). (ESI⁺) m/z: 224.7 (M+H)⁺, (C₉H₇BrN₂).

B. 3-(1-Oxo-5-(5-phenyl-1H-imidazol-4-yl) isoindolin-2-yl) piperidine-2,6-dione: To a solution of 3-(1-oxo-5-(4,4,5,5-tetramethyl-1,3,2-dioxaborolan-2-yl)isoindolin-2-yl)piperidine-2,6-dione (165 mg, 448 μmol, 2.00 eq) and 4-bromo-5-phenyl-1H-imidazole (50.0 mg, 224 μmol, 1.00 eq) in dioxane (3.00 mL) and H₂O (0.15 mL) were added K₃PO₄ (142 mg, 672 μmol, 3.00 eq) and Ru-Phos-Pd-G₃ (18.7 mg, 22.4 μmol, 0.10 eq) at 25° C. under N₂. Then the mixture was stirred at 100° C. for 2 h under N₂. The reaction mixture was poured into H₂O (15.0 mL) and extracted with ethyl acetate (3×10.0 mL). The combined organic layer was washed with brine (3×10.0 mL), dried over Na₂SO₄, filtered and concentrated to get residue. The residue was purified by preparative-HPLC (using a Phenomenex luna C18 (150×25 mm×10 μm) and gradient of 1.00%-26.0% acetonitrile in water containing 0.05% FA over 15 min at a flow rate of 25 mL/min to give the title compound (21.8 mg, 54.9 μmol, 24.5% yield, 97.0% purity in HPLC at 220 nm) was obtained as white solid. ¹H NMR: (400 MHz, MeOD) δ 8.05 (s, 1H), 7.91-7.85 (m, 1H), 7.72-7.70 (m, 1H), 7.66-7.59 (m, 1H), 7.45-7.43 (m, 2H), 7.38-7.32 (m, 3H), 5.17-5.12 (m, 1H), 4.51-4.40 (m, 2H), 2.91-2.86 (m, 1H), 2.80-2.79 (m, 1H), 2.51-2.46 (m, 1H), 2.19-2.18 (m, 1H). (ESI⁺) m/z: 387.1 (M+H)⁺, (C₂₂H₁₈N₄O₃).

### Example 14

### Synthesis of 3-(5-(1,2-Dimethyl-5-phenyl-1H-imidazol-4-yl)-1-oxoisoindolin-2-yl)piperidine-2,6-dione

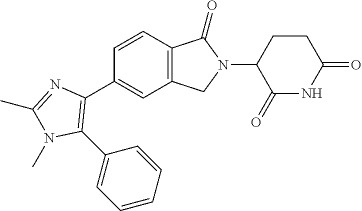 [SMILES: Cc1nc(-c2ccc3c(c2)CN(C2CCC(=O)NC2=O)C3=O)c(-c2ccccc2)n1C]

A. 1,2-Dimethyl-5-phenyl-1H-imidazole: A mixture of 1,2-dimethylimidazole (100 mg, 1.04 mmol, 1.00 eq), Pd(OAc)₂ (11.6 mg, 52.0 μmol, 0.05 eq), PCy₃ (29.1 mg, 104 μmol, 33.7 μL, 0.10 eq) and NaOBu-t (299 mg, 3.12 mmol, 3.00 eq) in o-xylene (5.00 mL) and chlorobenzene (351 mg, 3.12 mmol, 316 μL, 3.00 eq) was degassed and purged with N₂ for 3 times. Then the mixture was stirred at 130° C. for 12 h under N₂ atmosphere. After the reaction was completed, the reaction mixture was cooled to 25° C. and filtered. The filtrate was collected and dried under vacuum to give the title compound (170 mg, 977 μmol, 50.0% yield) as white solid. (ESI⁺) m/z: 172.1 (M+H)⁺, (C₁₁H₁₂N₂).

B. 4-Bromo-1,2-dimethyl-5-phenyl-1H-imidazole: To a solution of 1,2-dimethyl-5-phenyl-imidazole (140 mg, 812 μmol, 1.00 eq) in MeCN (8.00 mL) was added NBS (144 mg, 812 μmol, 1.00 eq) was stirred at 25° C. for 20 h. After the reaction was completed, the reaction mixture was filtered and the filtrate was concentrated in vacuum to give the title compound (150 mg, 597 μmol, 73.4% yield) as a yellow oil. ¹H NMR: (400 MHz, DMSO-d₆) δ 7.48 (t, J=7.6 Hz, 2H), 7.44-7.39 (m, 3H), 3.43 (s, 3H), 2.33 (s, 3H). (ESI⁺) m/z: 250.0 (M+H)⁺, (C₁₁H₁₁BrN₂).

C. 3-(5-(1,2-Dimethyl-5-phenyl-1H-imidazol-4-yl)-1-oxoisoindolin-2-yl)piperidine-2,6-dione: To a solution of 3-[1-oxo-5-(4,4,5,5-tetramethyl-1,3,2-dioxaborolan-2-yl)isoindolin-2-yl]piperidine-2,6-dione (0.20 g, 540 μmol, 1.00 eq) and 4-bromo-1,2-dimethyl-5-phenyl-imidazole (108 mg, 432 μmol, 0.80 eq) in dioxane (2.00 mL) and H₂O (0.10 mL) was added Ru-Phos-Pd-G₃ (45.1 mg, 54.0 μmol, 0.10 eq) and K₃PO₄ (229 mg, 1.08 mmol, 2.00 eq) under N₂. The reaction mixture was stirred at 100° C. for 1 h under N₂. The reaction mixture was concentrated in vacuum to get residue. The residue was purified by preparative-HPLC using a Welch Xtimate C18 (150 mm×25 mm 10 μm) and gradient of 3-33% acetonitrile in water containing 0.05% FA over 58 min at a flow rate of 20 mL/min to give the title compound (53.0 mg, 249 μmol, 25.0% yield, 97.8% purity in HPLC at 220 nm) as white solid. ¹H NMR: (400 MHz, DMSO-d₆) δ 10.9 (s, 1H), 7.59 (s, 1H), 7.53-7.47 (m, 4H), 7.39-7.36 (m, 3H), 5.08-5.03 (m, 1H), 4.35-4.16 (m, 2H), 3.39 (s, 3H), 2.92-2.85 (m, 1H), 2.58-2.50 (m, 1H), 2.41 (s, 3H), 2.37-2.36 (m, 1H), 1.97-1.94 (m, 1H). (ESI⁺) m/z: 415.2 (M+H)⁺, (C₂₄H₂₂N₄O₃).

### Example 15

### Synthesis of 3-(1-oxo-5-(5-phenyloxazol-4-yl)isoindolin-2-yl)piperidine-2,6-dione

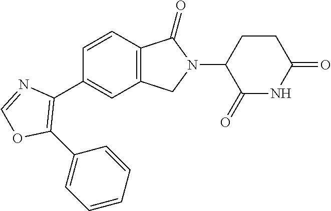 [SMILES: O=C1CCC(N2Cc3cc(-c4ncoc4-c4ccccc4)ccc3C2=O)C(=O)N1]

A. 3-(1-Oxo-5-(5-phenyloxazol-4-yl)isoindolin-2-yl)piperidine-2,6-dione: To a solution of 3-(1-oxo-5-(4,4,5,5-tetramethyl-1,3,2-dioxaborolan-2-yl)isoindolin-2-yl)piperidine-2,6-dione (248 mg, 670 μmol, 1.50 eq), 4-bromo-5-phenyloxazole (100 mg, 446 μmol, 1.00 eq) and K₃PO₄ (189 mg, 893 μmol, 2.00 eq) in dioxane (3.00 mL) and H₂O (0.15 mL) was added Ru-Phos-Pd-G₃ (74.7 mg, 893 μmol, 0.20 eq) under N₂. The mixture was stirred at 100° C. for 48 h under N₂. Then the reaction mixture was filtered to collect filtered liquid, and the filtered liquid was concentrated under reduced pressure to get residue. The residue was purified by preparative-HPLC (using a column: Welch Xtimate C18 (150×25 mm×5 μm) and gradient of 24.0%-54.0% acetonitrile in water containing 0.05% FA over 15 min at a flow rate of 25 mL/min to give the title compound (111 mg, 287 μmol, 64.0% yield, 99.7% purity in HPLC at 220 nm) as white solid. ¹H NMR: (400 MHz, DMSO-d₆) δ 11.0 (s, 1H), 8.60-8.58 (m, 2H), 7.95-7.83 (m, 1H), 7.60-7.58 (m, 1H), 7.58-7.54 (m, 1H), 7.51-7.50 (m, 2H), 7.55-7.45 (m, 2H), 5.15-5.10 (m, 1H), 4.37-4.35 (m, 1H), 4.34-4.33 (m, 1H), 2.95-2.87 (m, 1H), 2.62-2.58 (m, 1H), 2.41-2.37 (m, 1H), 2.03-2.00 (m, 1H). (ESI⁺) m/z: 387.1 (M+H)⁺, (C₂₂H₁₇N₃O₄).

### Example 16

### Synthesis of 3-(2-(1-methyl-5-phenyl-1H-imidazol-4-yl)-6-oxo-4,6-dihydro-5H-thieno[2,3-c]pyrrol-5-yl)piperidine-2,6-dione

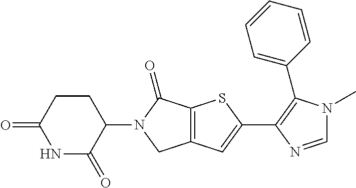 [SMILES: Cn1cnc(-c2cc3c(s2)C(=O)N(C2CCC(=O)NC2=O)C3)c1-c1ccccc1]

A. 5-Bromo-3-(((2,6-dioxopiperidin-3-yl)amino)methyl)thiophene-2-carboxylic acid: To a solution of tert-butyl 5-bromo-3-(((2,6-dioxo-3-piperidyl)amino)methyl)thiophene-2-carboxylate (2.00 g, 4.96 mmol, 1.00 eq) in DCM (80.0 mL) was added TFA (28.2 g, 247 mmol, 18.3 mL, 50.0 eq). The reaction mixture was stirred at 25° C. for 12 h. The reaction mixture was concentrated in vacuum to give the title compound (3.20 g, crude) as black oil. (ESI⁺) m/z: 348.9 (M+H)⁺, (C₁₁H₁₁BrN₂O₄S).

B. 3-(2-Bromo-6-oxo-4,6-dihydro-5H-thieno[2,3-c]pyrrol-5-yl)piperidine-2,6-dione: To a solution of 5-bromo-3-[[(2,6-dioxo-3-piperidyl)amino]methyl]thiophene-2-carboxylic acid (3.20 g, 9.22 mmol, 1.00 eq) in DMF (120 mL) was added HATU (4.38 g, 11.5 mmol, 1.25 eq) and DIEA (2.98 g, 23.0 mmol, 4.01 mL, 2.50 eq). The reaction mixture was stirred at 25° C. for 3 h. The reaction solvent was removed under vacuum to give a residue, which was diluted with H₂O (100 mL) and extracted with DCM (2×150 mL). The organic layers were dried over Na₂SO₄ and concentrated in vacuum to give residue. The residue was triturated with ethyl acetate (10.0 mL) at 0° C. for 2 h to give the title compound (1.10 g, 3.32 mmol, 36.4% yield, 99.4% purity in HPLC at 220 nm) as white solid. (ESI⁺) m/z: 328.9 (M+H)⁺, (C₁₁H₉BrN₂O₃S).

C. (5-(2,6-Dioxopiperidin-3-yl)-6-oxo-5,6-dihydro-4H-thieno[2,3-c]pyrrol-2-yl)boronic acid: To a solution of 3-(2-bromo-6-oxo-4H-thieno[2,3-c]pyrrol-5-yl)piperidine-2,6-dione (0.20 g, 607 μmol, 1.00 eq) in dioxane (4.00 mL) was added BPD (308 mg, 1.22 mmol, 2.00 eq), Pd(dppf)Cl₂ (111 mg, 151 μmol, 0.25 eq) and KOAc (119 mg, 1.22 mmol, 2.00 eq) under N₂. The reaction mixture was stirred at 90° C. for 16 h under N₂. The reaction mixture was concentrated in vacuum to give the title compound (0.50 g, crude) as white solid. (ESI⁺) m/z: 295.0 (M+H)⁺, (C₁₁H₁₁BN₂O₅S).

D. 1-Methyl-5-phenyl-1H-imidazole: To a solution of 5-phenyl-1H-imidazole (2.50 g, 17.3 mmol, 1.00 eq) in THF (50.0 mL) at 0° C., was added NaH (832 mg, 20.8 mmol, 60.0% purity, 1.20 eq) (5 batches) under N₂, The mixture was stirred at 0° C. for 30 min under N₂, then Mel (3.45 g, 24.3 mmol, 1.51 mL, 1.40 eq) was dropwise to the mixture. The reaction mixture was stirred at 25° C. for 2 h under N₂. The reaction mixture was poured into 100 mL H₂O under N₂, extracted with ethyl acetate (3×100 mL), washed with brine (50.0 mL), dried over Na₂SO₄ and concentrated to get a residue. The residue was purified by column chromatography (SiO₂, Petroleum ether:Ethyl acetate=10:1 to 0:1, Petroleum ether:Ethyl acetate=0:1, R_{f}=0.50) to give the title compound (0.78 g, 4.93 mmol, 32.0% yield) as yellow solid. (ESI⁺) m/z: 159.0 (M+H)⁺, (C₁₀H₁₀N₂).

E. 4-Bromo-1-methyl-5-phenyl-1H-imidazole: To a solution of 1-methyl-5-phenyl-imidazole (0.74 g, 4.68 mmol, 1.00 eq) in ACN (7.00 mL) was added NBS (874 mg, 4.91 mmol, 1.05 eq). The reaction mixture was stirred at 25° C. for 1 h. The reaction mixture was concentrated in vacuum to give residue. The residue was purified by preparative-TLC (SiO₂, Petroleum ether:Ethyl acetate=1:1; TLC, Petroleum ether:Ethyl acetate=1:1, R_{f}=0.25) to give the title compound (0.73 g, 3.08 mmol, 65.0% yield) as colorless oil. ¹H NMR: (400 MHz, CDCl₃) δ 7.50-7.21 (m, 6H), 3.58 (s, 3H). (ESI⁺) m/z: 236.6 (M+H)⁺, (C₁₀H₉BrN₂).

F. 3-(2-(1-Methyl-5-phenyl-1H-imidazol-4-yl)-6-oxo-4,6-dihydro-5H-thieno[2,3-c]pyrrol-5-yl)piperidine-2,6-dione: To a solution of 3-[6-oxo-2-(4,4,5,5-tetramethyl-1,3,2-dioxaborolan-2-yl)-4H-thieno[2,3-c]pyrrol-5-yl]piperidine-2,6-dione (0.20 g, 531 μmol, 1.00 eq) and 4-bromo-1-methyl-5-phenyl-imidazole (151 mg, 637 μmol, 1.20 eq) in dioxane (2.00 mL) and H₂O (0.10 mL) was added Ru-Phos-Pd-G₃ (88.9 mg, 106 μmol, 0.20 eq), K₃PO₄ (225 mg, 1.06 mmol, 2.00 eq) under N₂. The reaction mixture was stirred at 100° C. for 2 h under N₂. The reaction mixture was concentrated in vacuum to give residue. The residue was purified by preparative-HPLC (using a Welch Xtimate C18 (150 mm×25 mm 10 μm) and gradient of 40-70% acetonitrile in water containing 0.05% FA over 58 min at a flow rate of 25 mL/min to give the title compound (16.9 mg, 41.2 μmol, 14.0% yield, 99.1% purity in HPLC at 220 nm) as white solid. ¹H NMR: (400 MHz, DMSO-d₆) δ 10.9 (s, 1H), 7.83 (s, 1H), 7.59-7.55 (m, 3H), 7.51-7.49 (m, 2H), 6.74 (s, 1H), 4.96-4.91 (m, 1H), 4.23-4.10 (m, 2H), 3.46 (s, 3H), 2.89-2.53 (m, 1H), 2.33-2.26 (m, 2H), 1.97-1.95 (m, 1H). (ESI⁺) m/z: 407.1 (M+H)⁺, (C₂₁H₁IN₄O₃S).

### Example 17

### Synthesis of 3-(5-(1,5-Dimethyl-4-phenyl-1H-pyrazol-3-yl)-1-oxoisoindolin-2-yl)piperidine-2,6-dione

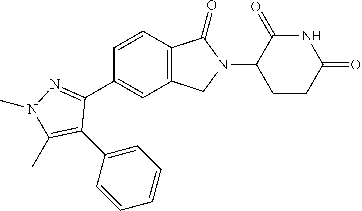 [SMILES: Cc1c(-c2ccccc2)c(-c2ccc3c(c2)CN(C2CCC(=O)NC2=O)C3=O)nn1C]

A. 3-Iodo-1,5-dimethyl-4-phenyl-1H-pyrazole: To a solution of 3-iodo-5-methyl-4-phenyl-1H-pyrazole (300 mg, 1.06 mmol, 1.00 eq) in DMF (3.00 mL) was added K₂CO₃ (292 mg, 2.11 mmol, 2.00 eq) and stirred for 30 min at 0° C. under N₂. Then CH₃I (180 mg, 1.27 mmol, 78.9 μL, 1.20 eq) was added to the reaction mixture. The reaction mixture was stirred for 3 h at 25° C. under N₂. The reaction mixture was poured into H₂O (20.0 mL) and extracted with EtOAc (3×15.0 mL). The combined organic were dried over Na₂SO₄, filtered and concentrated under reduced pressure to give a residue. The residue was purified by preparative-TLC (SiO₂, Petroleum ether:Ethyl acetate=5:1, R_{f}=0.30) to give the title compound (100 mg, 328 μmol, 31.1% yield, 97.8% purity in LCMS at 220 nm) as a white solid. (ESI⁺) m/z: 299.3 (M+H)⁺, (C₁₁H₁₁IN₂).

B. 3-(5-(1,5-Dimethyl-4-phenyl-1H-pyrazol-3-yl)-1-oxoisoindolin-2-yl)piperidine-2,6-dione: To a solution of 3-(1-oxo-5-(4,4,5,5-tetramethyl-1,3,2-dioxaborolan-2-yl)isoindolin-2-yl)piperidine-2,6-dione (102 mg, 276 μmol, 1.20 eq) and 3-iodo-1,5-dimethyl-4-phenyl-1H-pyrazole (70.0 mg, 229 μmol, 1.00 eq) in dioxane (1.00 mL) and H₂O (0.05 mL) was added K₃PO₄ (146 mg, 688 μmol, 3.00 eq) and Ru-Phos-Pd-G₃ (19.2 mg, 22.9 μmol, 0.10 eq) under N₂. The mixture was stirred at 100° C. under N₂ for 10 h.

Concentrated under reduced pressure to give a residue. The residue was purified by preparative-HPLC (using a Phenomenex luna C18 (150×25 mm×10 μm) and gradient of 25.0%-55.0% acetonitrile in water containing 0.05% FA over 13 min at a flow rate of 25 mL/min to give the title compound (9.40 mg, 22.7 μmol, 9.00% yield, 98.7% purity in HPLC at 220 nm) was obtained as white solid. ¹H NMR: (400 MHz, DMSO-d₆) δ 11.0-10.9 (m, 1H), 7.58-7.56 (m, 2H), 7.41-7.36 (m, 3H), 7.33-7.31 (m, 1H), 7.18-7.16 (m, 2H), 5.10-5.06 (m, 1H), 4.39-4.35 (m, 1H), 4.25-4.21 (m, 1H), 3.85 (s, 3H), 2.94-2.86 (m, 1H), 2.72-2.61 (m, 1H), 2.43-2.34 (m, 1H), 2.26-2.22 (m, 3H), 2.05-1.95 (m, 1H). (ESI⁺) m/z: 414.9 (M+H)⁺, (C₂₄H₂₂N₄O₃).

### Example 18

### Synthesis of 3-(5-(1, 3-Dimethyl-4-phenyl-1H-pyrazol-5-yl)-1-oxoisoindolin-2-yl)piperidine-2,6-dione

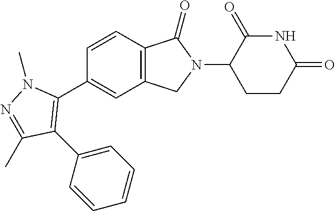 [SMILES: Cc1nn(C)c(-c2ccc3c(c2)CN(C2CCC(=O)NC2=O)C3=O)c1-c1ccccc1]

A. 5-Iodo-1,3-dimethyl-4-phenyl-1H-pyrazole: To a solution 3-iodo-5-methyl-4-phenyl-1H-pyrazole (300 mg, 1.06 mmol, 1.00 eq) in DMF (3.00 mL) was added K₂CO₃ (292 mg, 2.11 mmol, 2.00 eq) and stirred for 30 min at 0° C. under N₂. Then CH₃I (180 mg, 1.27 mmol, 78.9 μL, 1.20 eq) was added to the reaction mixture. The reaction mixture was stirred for 3 h at 25° C. under N₂. The reaction mixture was poured into H₂O (20.0 mL) and extracted with EtOAc (3×15.0 mL). The combined organic layers were dried over Na₂SO₄, filtered and concentrated under reduced pressure to give a residue. The residue was purified by preparative-TLC (SiO₂, Petroleum ether:Ethyl acetate=5:1, R_{f}=0.40) to give the title compound (80.0 mg, 258 μmol, 24.4% yield, 96.1% purity in LCMS at 220 nm) as a white solid. (ESI⁺) m/z: 299.3 (M+H)⁺, (C₁₁H₁₁IN₂).

B. 3-(5-(1,3-Dimethyl-4-phenyl-1H-pyrazol-5-yl)-1-oxoisoindolin-2-yl)piperidine-2,6-dione: To a solution of 3-(1-oxo-5-(4,4,5,5-tetramethyl-1,3,2-dioxaborolan-2-yl)isoindolin-2-yl)piperidine-2,6-dione (104 mg, 282 μmol, 1.20 eq) and 5-iodo-1,3-dimethyl-4-phenyl-1H-pyrazole (73.0 mg, 235 μmol, 1.00 eq) in dioxane (1.00 mL) and H₂O (0.05 mL) was added K₃PO₄ (150 mg, 704 μmol, 3.00 eq) and Ru-Phos-Pd-G₃ (19.6 mg, 23.5 μmol, 0.10 eq) under N₂. The mixture was stirred at 100° C. under N₂ for 2 h. Concentrated under reduced pressure to give a residue. The residue was purified by preparative-HPLC (using a Phenomenex luna C18 (150×25 mm×10 μm) and gradient of 24.0%-54.0% acetonitrile in water containing 0.05% FA over 13 min at a flow rate of 25 mL/min to give the title compound (1.14 mg, 2.75 μmol, 1.00% yield, 97.1% purity in HPLC at 220 nm) was obtained as white solid. ¹HNMR: (400 MHz, DMSO-d₆) δ 11.07-10.9 (m, 1H), 7.73-7.71 (m, 1H), 7.58 (s, 1H), 7.46-7.40 (m, 1H), 7.35-7.33 (m, 2H), 7.27-7.18 (m, 1H), 7.09-7.07 (m, 2H), 5.14-5.10 (m, 1H), 4.48-4.44 (m, 1H), 4.35-4.31 (m, 1H), 3.70 (s, 3H), 2.93-2.88 (m, 1H), 2.62-2.61 (m, 1H), 2.41-2.38 (m, 1H), 2.21 (s, 3H), 2.06-1.96 (m, 1H). (ESI⁺) m/z: 415.0 (M+H)⁺, (C₂₄H₂₂N₄O₃).

### Example 19

### Synthesis of 3-(5-(2-Methyl-5-phenyloxazol-4-yl)-1-oxoisoindolin-2-yl)piperidine-2,6-dione

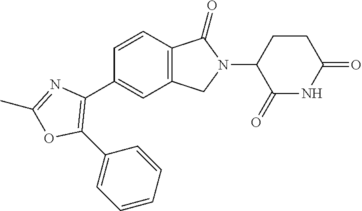 [SMILES: Cc1nc(-c2ccc3c(c2)CN(C2CCC(=O)NC2=O)C3=O)c(-c2ccccc2)o1]

A. N-(2-Oxo-2-phenylethyl)acetamide: To a solution of 2-amino-1-phenyl-ethanone (10.0 g, 58.2 mmol, 1.00 eq, HCl) in THF (300 mL) was added acetyl acetate (16.1 g, 158 mmol, 14.8 mL, 2.71 eq) and TEA (11.7 g, 116 mmol, 16.2 mL, 2.00 eq). The mixture was stirred at 25° C. for 2 h. After the reaction was completed, the reaction mixture was concentrated under reduced pressure to give the title compound (10.0 g, crude) as a brown oil. ¹H NMR: (400 MHz, DMSO-d₆) δ 8.27 (t, J=5.2 Hz, 1H), 7.98 (d, J=7.2 Hz, 2H), 7.65 (t, J=7.6 Hz, 1H), 7.53 (t, J=7.6 Hz, 2H), 4.59 (d, J=5.6 Hz, 2H), 1.91 (s, 3H). (ESI⁺) m/z: 177.0 (M+H)⁺, (C₁₀H₁₁NO₂).

B. 2-Methyl-5-phenyloxazole: Compound N-phenacylacetamide (10.0 g, 56.0 mmol, 1.00 eq) was added to H₂SO₄ (10.0 mL), the mixture was stirred at 80° C. for 2 h. The reaction mixture was cooled down to 25° C. and poured into ice cold water (50.0 mL). The solution was neutralized with 28% aqueous ammonia and extracted with ethyl acetate (3×20.0 mL). The organic layer was concentrated under reduced pressure to give the title compound (0.30 g, crude) as yellow solid. (ESI⁺) m/z: 160.0 (M+H)⁺, (C₁₀H₉NO).

C. 4-Bromo-2-methyl-5-phenyloxazole: To a solution of 2-methyl-5-phenyl-oxazole (0.30 g, 1.90 mmol, 1.00 eq) in ACN (10.0 mL) was added NBS (1.40 g, 8.17 mmol, 1.30 eq) at 0° C. The reaction mixture was stirred at 85° C. for 3 h. The reaction mixture was concentrated under reduced pressure to give a residue. The residue was purified by column chromatography (SiO₂, Petroleum ether:Ethyl acetate=10:1; TLC, Petroleum ether:Ethyl acetate=3:1, R_{f}=0.50) to give the title compound (0.30 g, 1.28 mmol, 66.0% yield) as yellow solid. ¹H NMR: (400 MHz, CDCl₃) δ 7.90 (d, J=8.0 Hz, 2H), 7.45 (t, J=3.6 Hz, 2H), 7.37-7.27 (m, 1H), 2.54 (s, 3H). (ESI⁺) m/z: 237.9 (M+H)⁺, (C₁₀H₈BrNO).

D. 3-(5-(2-Methyl-5-phenyloxazol-4-yl)-1-oxoisoindolin-2-yl)piperidine-2,6-dione: To a solution of 3-[1-oxo-5-(4,4,5,5-tetramethyl-1,3,2-dioxaborolan-2-yl)isoindolin-2-yl]piperidine-2,6-dione (0.20 g, 540 μmol, 1.00 eq) and 4-bromo-2-methyl-5-phenyl-oxazole (154 mg, 648 μmol, 1.20 eq) in dioxane (2.00 mL) and H₂O (0.10 mL) was added Ru-Phos-Pd-G₃ (90.3 mg, 108 μmol, 0.20 eq), K₃PO₄ (229 mg, 1.08 mmol, 2.00 eq) under N₂. The reaction mixture was stirred at 100° C. for 2 h under N₂. The reaction mixture was concentrated in vacuum to get residue. The residue was purified by preparative-HPLC using a Welch Xtimate C18 (150 mm×25 mm 10 μm) and gradient of 27-57% acetonitrile in water containing 0.05% FA over 58 min at a flow rate of 20 mL/min to give the title compound (75.3 mg, 170 μmol, 30.0% yield, 90.7% purity in HPLC at 220 nm) as white solid. ¹H NMR: (400 MHz, DMSO-d₆) δ 10.9 (s, 1H), 7.80 (s, 1H), 7.76-7.72 (m, 2H), 7.56-7.54 (m, 2H), 7.46-7.44 (m, 3H), 5.14-5.09 (m, 1H), 4.49-4.31 (m, 2H), 2.94-2.88 (m, 1H), 2.56-2.54 (m, 1H), 2.53 (s, 3H), 2.41-2.38 (m, 1H), 2.36-2.01 (m, 1H). (ESI⁺) m/z: 402.1 (M+H)⁺, (C₂₃H₁₉N₃O₄).

### Example 20

### Synthesis of 3-(7-(1-Methyl-5-phenyl-1H-imidazol-4-yl)-3-oxo-[1,2,4]triazolo[4,3-a]pyridin-2 (3H)-yl)piperidine-2,6-dione

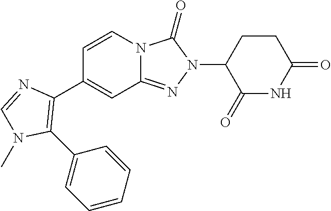 [SMILES: Cn1cnc(-c2ccn3c(=O)n(C4CCC(=O)NC4=O)nc3c2)c1-c1ccccc1]

A. 3-(7-(1-Methyl-5-phenyl-1H-imidazol-4-yl)-3-oxo-[1,2,4]triazolo[4,3-a]pyridin-2 (3H)-yl)piperidine-2,6-dione: To a solution of 3-(3-oxo-7-(4,4,5,5-tetramethyl-1,3,2-dioxaborolan-2-yl)-[1,2,4]triazolo[4,3-a]pyridin-2-yl)piperidine-2,6-dione (crude, 120 mg, 322 μmol, 1.00 eq), 4-bromo-1-methyl-5-phenyl-1H-imidazole (91.7 mg, 386 μmol, 1.20 eq) and K₃PO₄ (136 mg, 644 μmol, 2.00 eq) in dioxane (2.00 mL) and H₂O (0.10 mL) was added Ru-Phos-Pd-G₃ (26.9 mg, 32.2 μmol, 0.10 eq) under N₂. The reaction mixture was stirred at 100° C. for 2 h under N₂. Then the reaction mixture was filtered through diatomite and the filtrate was concentrated under vacuum to give a crude product. The crude product was purified by reversed-phase HPLC (0.1% HCl condition) to give the title compound (12.8 mg, 31.8 μmol, 7.69% yield, >99% purity in HPLC at 220 nm) as a yellow solid. ¹H NMR (400 MHz, DMSO-d₆): δ 11.1 (s, 1H), 8.98 (s, 1H), 7.88 (d, J=7.2 Hz, 1H), 7.64-7.59 (m, 3H), 7.57-7.52 (m, 2H), 7.21 (s, 1H), 6.47 (d, J=7.2 Hz, 1H), 5.35 (dd, J=12.8, 5.2 Hz, 1H), 3.59 (s, 3H), 2.94-2.84 (m, 1H), 2.65-2.58 (m, 1H), 2.49-2.43 (m, 1H), 2.18-2.10 (m, 1H). (ESI⁺) m/z: 403.2 (M+H)⁺, (C₂₁H₁₈N₆O₃).

### Example 21

### Synthesis of 3-(5-(1-Methyl-4-phenyl-1H-imidazol-5-yl)-1-oxoisoindolin-2-yl)piperidine-2,6-dione

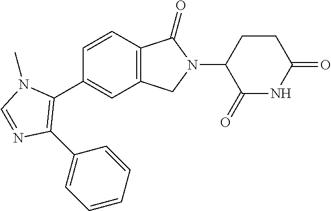 [SMILES: Cn1cnc(-c2ccccc2)c1-c1ccc2c(c1)CN(C1CCC(=O)NC1=O)C2=O]

A. 3-(5-(1-Methyl-4-phenyl-1H-imidazol-5-yl)-1-oxoisoindolin-2-yl)piperidine-2,6-dione: To a solution of 3-[1-oxo-5-(4,4,5,5-tetramethyl-1,3,2-dioxaborolan-2-yl)isoindolin-2-yl]piperidine-2,6-dione (0.10 g, 270 μmol, 1.00 eq) and 5-bromo-1-methyl-4-phenyl-imidazole (76.5 mg, 324 μmol, 1.20 eq) in dioxane (3.00 mL) and H₂O (0.15 mL) was added Ru-Phos-Pd-G₃ (22.5 mg, 17.0. μmol, 0.10 eq) and K₃PO₄ (114 mg, 540 μmol, 2.00 eq) under N₂. The reaction mixture was stirred at 100° C. for 2 h under N₂. The reaction mixture was concentrated in vacuum to give residue. The residue was purified by preparative-HPLC (using a Welch Xtimate C18 (150 mm×25 mm 10 μm) and gradient of 1-31% acetonitrile in water containing 0.05% FA over 58 min at a flow rate of 25 mL/min to give the title compound (65.0 mg, 81.0 μmol, 60.0% yield, 100% purity in HPLC at 220 nm) as white solid. ¹H NMR: (400 MHz, DMSO-d₆) δ 11.0 (s, 1H), 7.83-7.81 (m, 2H), 7.67 (s, 1H), 7.31-7.29 (m, 2H), 7.50-7.48 (m, 1H), 7.39-7.37 (m, 2H), 5.17-5.12 (m, 1H), 4.53-4.36 (m, 2H), 3.50 (s, 3H), 2.93-2.87 (m, 1H), 2.65-2.62 (m, 1H), 2.42-2.39 (m, 1H), 2.05-2.02 (m, 1H). (ESI⁺) m/z: 401.2 (M+H)⁺, (C₂₃H₂₀N₄O₃).

### Example 22

### Synthesis of 3-(5-(1-Isobutyl-5-phenyl-1H-imidazol-4-yl)-1-oxoisoindolin-2-yl)piperidine-2,6-dione

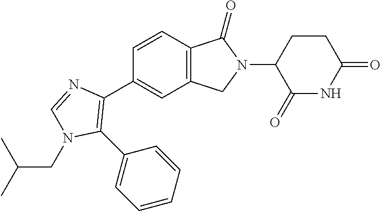 [SMILES: CC(C)Cn1cnc(-c2ccc3c(c2)CN(C2CCC(=O)NC2=O)C3=O)c1-c1ccccc1]

A. 1-Isobutyl-5-phenyl-1H-imidazole: To a solution of (E)-N-(2-phenyl-1-tosylvinyl)formamide (2.00 g, 6.63 mmol, 1.00 eq) in DME (20.0 mL) was added TEA (3.35 g, 33.1 mmol, 4.61 mL, 5.00 eq) at −5° C. Then POCl₃ (0.81 g, 5.30 mmol, 492 μL, 0.80 eq) was added dropwise at −5° C. The mixture was added 2-methylpropan-1-amine (1.03 g, 14.1 mmol, 1.40 mL, 2.00 eq). Then the mixture was stirred at 25° C. for 5 h. The mixture was poured into water (100 mL) at 0° C. and extracted with DCM (3×80.0 mL). Combined organic were dried over Na₂SO₄, filtered and concentrated under reduced pressure to give a residue. The residue was purified by column chromatography (SiO₂, Petroleum ether:Ethyl acetate=10:1, TLC:Dichloromethane:Methanol=10:1,R_{f}=0.2) to give the title compound (400 mg, 2.00 mmol, 30.1% yield) as brown oil. (ESI⁺) m/z: 201.0 (M+H)⁺, (C₁₃H₁₆N₂).

B. 4-Bromo-1-isobutyl-5-phenyl-1H-imidazole: To a solution of 1-isobutyl-5-phenyl-1H-imidazole (400 mg, 2.00 mmol, 1.00 eq) in ACN (20.0 mL) was added NBS (249 mg, 1.40 mmol, 0.70 eq) slowly at 0° C. Then the mixture was stirred at 25° C. for 2 h. Then the mixture was poured into H₂O (20.0 mL) and extracted with DCM (3×15.0 mL). The combined organic layers were washed with saturated NaCl aqueous (3×15.0 mL), dried over Na₂SO₄ and filtered to collect filtered liquid. The liquid was concentrated under reduced pressure to get residue. The residue was purified by preparative-TLC (DCM:MeOH=10:1, R_{f}=0.55) to get the title compound (500 mg, 1.69 mmol, 84.3% yield, 94.1% purity in LCMS at 220 nm) as white solid. ¹H NMR: (400 MHz, DMSO-d₆) δ 7.80 (s, 1H), 7.52-7.40 (m, 5H), 3.79-3.77 (m, 2H), 1.63-1.53 (m, 1H), 0.64-0.61 (m, 6H). (ESI⁺) m/z: 279.0 (M+H)⁺, (C₁₃H₁₅BrN₂).

C. 3-(5-(1-Isobutyl-5-phenyl-1H-imidazol-4-yl)-1-oxoisoindolin-2-yl)piperidine-2,6-dione: To a solution of 4-bromo-1-isobutyl-5-phenyl-1H-imidazole (300 mg, 1.07 mmol, 1.00 eq), 3-(1-oxo-5-(4,4,5,5-tetramethyl-1,3,2-dioxaborolan-2-yl)isoindolin-2-yl)piperidine-2,6-dione (517 mg, 1.40 mmol, 1.30 eq) and K₃PO₄ (456 mg, 2.15 mmol, 2.00 eq) in dioxane (6.00 mL) and H₂O (0.30 mL) was added Ru-Phos-Pd-G₃, (89.9 mg, 107 μmol, 0.10 eq) under N₂. The mixture was stirred at 100° C. for 4 h under N₂. The mixture was poured into H₂O (20.0 mL) and extracted with DCM (3×15.0 mL). The combined organic layers were washed with saturated NaCl aqueous (3×15.0 mL), dried over Na₂SO₄ and filtered to collect filtered liquid. The liquid was concentrated under reduced pressure to get residue. The residue was purified by preparative-HPLC using a column: Welch Xtimate C18 (150×25 mm×5 μm) and gradient of 11.0%-41.0% acetonitrile in water containing 0.05% FA over 15 min at a flow rate of 25 mL/min to give the title compound (125 mg, 279 μmol, 25.9% yield, 98.7% purity in HPLC at 220 nm) as white solid. ¹H NMR: (400 MHz, DMSO-d₆) δ 10.9 (s, 1H), 7.89 (s, 1H), 7.59-7.53 (m, 1H), 7.52-7.46 (m, 5H), 7.43-7.39 (m, 2H), 5.08-5.04 (m, 1H), 4.36-4.31 (m, 2H), 3.70-3.66 (m, 2H), 2.92-2.86 (m, 1H), 2.54-2.52 (m, 1H), 2.37-2.34 (m, 1H), 1.98-1.96 (m, 1H), 1.66-1.63 (m, 6H). (ESI⁺) m/z: 443.0 (M+H)⁺, (C₂₆H₂₆N₄

### Example 23

### Synthesis of 3-(1-Oxo-5-(5-phenylthiazol-4-yl) isoindolin-2-yl) piperidine-2, 6-dione

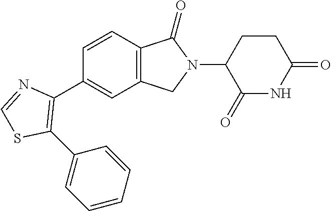 [SMILES: O=C1CCC(N2Cc3cc(-c4ncsc4-c4ccccc4)ccc3C2=O)C(=O)N1]

A. 4-Bromo-5-phenylthiazole: To a solution of 5-phenylthiazole (0.50 g, 3.10 mmol, 1.00 eq) in ACN (5.00 mL) was added NBS (607 mg, 3.41 mmol, 1.10 eq) under N₂ at 25° C. The mixture was stirred at 50° C. for 1 h. The reaction mixture was poured into H₂O (15.0 mL) and extracted with Ethyl acetate (3×10.0 mL). The combined organic layer was washed with brine (3×10.0 mL), dried over Na₂SO₄, filtered and concentrated to get residue. The residue was purified by column chromatography (SiO₂, Petroleum ether:Ethyl acetate=10:1, TLC:Petroleum ether:Ethyl acetate=3:1,R_{f}=0.30) to give the title compound (563 mg, 2.34 mmol, 75.6% yield, 100% purity in LCMS at 220 nm) as light yellow solid. ¹H NMR: (400 MHz, CDCl₃) δ 8.74 (s, 1H), 7.66-7.64 (m, 2H), 7.46-7.44 (m, 3H). (ESI⁺) m/z: 241.8 (M+H)⁺, (C₉H₆BrNS).

B. 3-(1-Oxo-5-(5-phenylthiazol-4-yl)isoindolin-2-yl)piperidine-2,6-dione: To a solution of 3-(1-oxo-5-(4,4,5,5-tetramethyl-1,3,2-dioxaborolan-2-yl)isoindolin-2-yl)piperidine-2,6-dione (185 mg, 499 μmol, 1.20 eq) and 4-bromo-5-phenylthiazole (100 mg, 416 μmol, 1.00 eq) in dioxane (2.00 mL) and H₂O (0.10 mL) was added Ru-Phos-Pd-G₃ (34.8 mg, 41.6 μmol, 0.10 eq) and K₃PO₄ (176.8 mg, 832 μmol, 2.00 eq) under N₂ at 25° C. The mixture was stirred at 100° C. for 2 h under N₂. The reaction mixture was concentrated under reduced pressure to get a residue at 45° C. The residue was purified by preparative-HPLC (using a Phenomenex luna C18 (150×25 mm×7 μm) and gradient of 26.0%-56.0% acetonitrile in water containing 0.05% FA over 20 mins at a flow rate of 25 mL/min to give the title compound (102.1 mg, 205 mmol, 60.1% yield, 98.9% purity in HPLC at 220 nm) was obtained as white solid. ¹H NMR: (400 MHz, DMSO-d₆) δ 10.9 (s, 1H), 9.24 (s, 1H), 7.73-7.72 (m, 1H), 7.65-7.63 (m, 1H), 7.52-7.46 (m, 1H), 7.43-7.37 (m, 5H), 5.12-5.08 (m, 1H), 4.44-4.26 (m, 2H), 2.93-2.87 (m, 1H), 2.61-2.56 (m, 1H), 2.40-2.36 (m, 1H), 2.02-2.00 (m, 1H). (ESI⁺) m/z: 404.0 (M+H)⁺, (C₂₂H₁₇N₃O₃S).

### Example 24

### Synthesis of (R)-3-(5-(5-(4-fluorophenyl)-1-methyl-1H-imidazol-4-yl)-1-oxoisoindolin-2-yl) piperidine-2, 6-dione

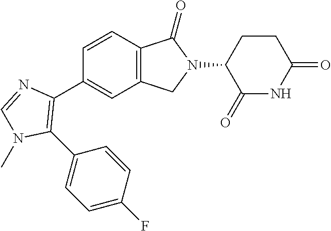 [SMILES: Cn1cnc(-c2ccc3c(c2)CN([C@@H]2CCC(=O)NC2=O)C3=O)c1-c1ccc(F)cc1]

A. (S)-tert-butyl 5-amino-4-(5-bromo-1-oxoisoindolin-2-yl)-5-oxopentanoate: To a suspension of tert-butyl (4S)-4,5-diamino-5-oxo-pentanoate (14.5 g, 60.6 mmol, 1.00 eq, HCl) in MeCN (231 mL) was added DIEA (28.0 g, 2171 mmol, 37.7 mL, 3.57 eq) at 0° C. After stirring for 15 min, the methyl 4-bromo-2-(bromomethyl)benzoate (22.0 g, 71.4 mmol, 1.18 eq) was added into above the mixture in several portions over 15 min. The reaction mixture was stirred at 0° C. for 30 min and warmed to 25° C. for 3 h. After the reaction mixture was warmed to 60° C. for 12 h. The mixture was cooled to 25° C. 50 mL water was added and the mixture was stirred at 25° C. for 30 min. The resultant solid was filtered and washed with 20 ml EtOAc. The solid was dried under vacuum filtration to give the title compound (18.0 g, 45.3 mmol, 74.8% yield) as yellow solid. ¹H NMR (400 MHz, DMSO-d₆): δ 7.88 (s, 1H), 7.69-7.67 (m, 2H), 7.57-7.67 (m, 1H), 7.19 (s, 1H), 4.74-4.71 (m, 1H), 4.71-4.44 (m, 2H), 2.19-2.14 (m, 3H), 2.00-1.97 (m, 1H), 1.33 (s, 9H). (ESI⁺) m/z: 396.1 (M+H)⁺, (C₁₇H₂₁BrNO₄).

B. (S)-tert-Butyl 5-amino-5-oxo-4-(1-oxo-5-(4,4,5,5-tetramethyl-1,3,2-dioxaborolan-2-yl)isoindolin-2-yl)pentanoate: To a solution of (S)-tert-butyl 5-amino-5-oxo-4-(1-oxo-5-(4,4,5,5-tetramethyl-1,3,2-dioxaborolan-2-yl)isoindolin-2-yl)pentanoate (10.0 g, 25.2 mmol, 1.00 eq), BPD (7.67 g, 30.2 mmol, 1.20 eq) in dioxane (100 mL) was added AcOK (7.41 g, 75.52 mmol, 3.00 eq), Pd(dppf)Cl₂ (1.01 g, 1.38 mmol, 0.05 eq). The mixture was stirred at 60° C. for 2 h under N₂. The reaction mixture was diluted with EtOAc (50.0 mL), filtered through a plug of celite, and washed with additional EtOAc (30.0 mL). The filtrate was dried under vacuum to give the title compound (8.74 g, 14.5 mmol, 57% yield, 73.8% purity in LCMS at 220 nm) as off-white solid. (ESI⁺) m/z: 445.1 (M+H)⁺, (C₂₂H₃₃BN₂O₆).

C. tert-Butyl (S)-5-amino-4-(5-(1-methyl-1H-imidazol-4-yl)-1-oxoisoindolin-2-yl)-5-oxopentanoate: To a solution of tert-butyl (S)-5-amino-5-oxo-4-(1-oxo-5-(4,4,5,5-tetramethyl-1,3,2-dioxaborolan-2-yl)isoindolin-2-yl)pentanoate (117 mg, 264 μmol, 1.10 eq) and 4-iodo-1-methyl-1H-imidazole (50.0 mg, 240 μmol, 1.00 eq) in dioxane (1.00 mL) and H₂O (0.10 mL) was added K₃PO₄ (153 mg, 721 μmol, 3.00 eq) and cataCXium A Pd G₃ (17.5 mg, 24.0 μmol, 0.1 eq) at 25° C. under N₂. The mixture was stirred at 80° C. for 10 h under N₂. The mixture was concentrated under reduced pressure to give a residue. The residue was poured into H₂O (5.0 mL) and extracted with DCM (3×10 mL) to collected the organic layer. The organic layer were dried over Na₂SO₄, filtered and concentrated under vacuum at 40° C. to get a residue. The residue was purified by Prep-TLC (Dichloromethane:Methanol=10:1, R_{f}=0.20) to give the title compound (40.0 mg, 72.8 μmol, 32.0% yield, 85.0% purity in LCMS at 220 nm) as brown solid. (ESI⁺) m/z: 399.1 (M+H)⁺, (C₂₁H₂₆N₄O₄).

D. tert-Butyl (S)-5-amino-4-(5-(5-bromo-1-methyl-1H-imidazol-4-yl)-1-oxoisoindolin-2-yl)-5-oxopentanoate: To a solution of tert-butyl (S)-5-amino-4-(5-(1-methyl-1H-imidazol-4-yl)-1-oxoisoindolin-2-yl)-5-oxopentanoate (40.0 mg, 72.1 μmol, 1.00 eq) in MeCN (1.00 mL) was added NBS (12.8 mg, 72.0 μmol, 1.00 eq) at 0° C. Then the mixture was stirred at 25° C. for 2 h. The mixture was concentrated under reduced pressure to give a residue. The residue was purified by Prep-TLC (Dichloromethane:Methanol=10:1, R_{f}=0.25) to give the title compound (15.0 mg, 30.9 μmol, 53.0% yield, 98.6% purity in LCMS at 220 nm) as brown solid. ¹H NMR (400 MHz, DMSO-d₆): δ 8.11-8.08 (m, 1H), 8.05-8.00 (m, 2H), 7.75-7.23 (m, 1H), 7.56 (s, 1H), 7.19 (s, 1H), 4.76-4.72 (m, 1H), 4.61-4.48 (m, 2H), 3.66 (s, 3H), 2.19-2.14 (m, 3H), 2.00-1.97 (m, 1H), 1.33 (s, 9H). (ESI⁺) m/z: 476.1 (M+H)⁺, (C₂₁H₂₅BrN₄O₄).

E. tert-Butyl (S)-5-amino-4-(5-(5-(4-fluorophenyl)-1-methyl-1H-imidazol-4-yl)-1-oxoisoindolin-2-yl)-5-oxopentanoate: To a solution of tert-butyl (S)-5-amino-4-(5-(5-bromo-1-methyl-1H-imidazol-4-yl)-1-oxoisoindolin-2-yl)-5-oxopentanoate (15.0 mg, 30.9 μmol, 1.00 eq), 2-(4-fluorophenyl)-4,4,5,5-tetramethyl-1,3,2-dioxaborolane (8.67 mg, 61.9 μmol, 2.00 eq) and K₂CO₃ (8.56 mg, 61.9 μmol, 2.00 eq) in dioxane (1.00 mL) was added Pd(dppf)Cl₂ (7.16 mg, 6.20 μmol, 0.20 eq) under N₂. Then the reaction mixture was stirred at 100° C. for 2 h under N₂. The mixture was poured into H₂O (6.0 mL) and extracted with DCM (3×10.0 mL) to collected the organic layer. The organic layer were dried over Na₂SO₄, filtered and the filtrated was concentrated under vacuum at 40° C. to get a residue. The residue was purified by Prep-TLC (Dichloromethane:Methanol=10:1, R_{f}=0.15) to give the title compound (10.0 mg, 19.8 μmol, 60.0% yield, 97.5% purity in LCMS at 220 nm) as white solid. (ESI⁺) m/z: 493.3 (M+H)⁺, (C₂₇H₂₉FN₄O₄).

F. (R)-3-(5-(5-(4-Fluorophenyl)-1-methyl-1H-imidazol-4-yl)-1-oxoisoindolin-2-yl) piperidine-2, 6-dione: To a solution of tert-butyl (S)-5-amino-4-(5-(5-(4-fluorophenyl)-1-methyl-1H-imidazol-4-yl)-1-oxoisoindolin-2-yl)-5-oxopentanoate (10.0 mg, 19.8 μmol, 1.00 eq) in MeCN (1.00 mL) was added TsOH (17.0 mg, 98.9 μmol, 5.00 eq). The mixture was stirred at 80° C. for 4 h. The mixture was poured into H₂O (10.0 mL) and extracted with DCM (3×10.0 mL). The combined organic layers were washed with brine (2×15.0 mL), dried over Na₂SO₄, filtered and concentrated under reduced pressure to get residue. The residue was purified by preparative-HPLC using a Phenomenex luna C18 (150×25 mm×5 um) and gradient of 4-34% acetonitrile in water containing 0.05% FA over 15 min at a flow rate of 25 mL/min to give the title compound (2.57 mg, 5.96 μmol, 29.4% yield, 97.1% purity in HPLC at 220 nm) as white solid. ¹H NMR (400 MHz, DMSO-d₆): δ 10.9-10.8 (m, 1H), 7.85 (s, 1H), 7.59-7.55 (m, 1H), 7.53-7.52 (m, 1H), 7.48-7.47 (m, 1H), 7.46-7.45 (m, 3H), 7.44-7.41 (m, 2H), 5.09-5.04 (m, 1H), 4.30-4.25 (m, 1H), 4.20-4.15 (m, 1H), 3.47 (s, 3H), 2.95-2.86 (m, 1H), 2.55-2.54 (m, 1H), 2.37-2.34 (m, 1H), 2.32-1.90 (m, 1H). (ESI⁺) m/z: 418.1 (M+H)⁺, (C₂₃H₁₉FN₄O₃).

### Example 25

### Synthesis of 3-(5-(1-Methyl-2-phenyl-1H-imidazol-4-yl)-1-oxoisoindolin-2-yl)piperidine-2,6-dione

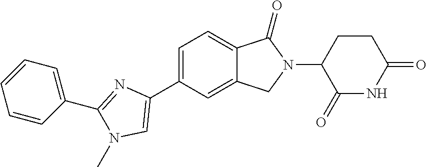 [SMILES: Cn1cc(-c2ccc3c(c2)CN(C2CCC(=O)NC2=O)C3=O)nc1-c1ccccc1]

A. 4,5-Dibromo-2-phenyl-1H-imidazole: To a solution of 2-phenyl-1H-imidazole (10.0 g, 69.3 mmol, 1.00 eq) in DMF (100 mL) was added NBS (24.6 g, 138 mmol, 2.00 eq) at 0° C. The reaction mixture was stirred at 25° C. for 1 h. The reaction mixture was concentrated under reduce pressure to give a residue. To the residue was added H₂O (100 mL) and extracted with ethyl acetate (3×100 mL), and the combined organic layer was washed with saturated aq. Na₂SO₃ (2×100 mL), dried over anhydrous Na₂SO₄. The organic layer was concentrated in vacuum to give the title compound (6.00 g, crude) as yellow solid. (ESI⁺) m/z: 300.8 (M+H)⁺, (C₉H₆Br₂N₂).

B. 4-Bromo-2-phenyl-1H-imidazole: To a solution of 4,5-dibromo-2-phenyl-1H-imidazole (5.00 g, 16.5 mmol, 1.00 eq) in EtOH (50.0 mL) and H₂O (50.0 mL) was added Na₂S₂O₃ (10.4 g, 66.2 mmol, 4.00 eq) under N₂. The reaction mixture was stirred at 110° C. for 96 h under N₂. The mixture was cooled at 25° C., and the solvent was removed under reduced pressure to give residue. The residue was purified by column chromatography (SiO₂, Petroleum ether:Ethyl acetate=100:1 to 5:1; TLC, Petroleum ether:Ethyl acetate=3:1, R_{f}=0.40) to give the title compound (1.10 g, 4.88 mmol, 25.0% yield, 98.9% purity in HPLC at 220 nm) as yellow solid. ¹H NMR: (400 MHz, DMSO-d₆) δ 12.8 (s, 1H), 7.88 (d, J=7.2 Hz, 2H), 7.45-7.41 (m, 2H), 7.40-7.38 (m, 1H), 4.37-4.35 (m, 1H). (ESI⁺) m/z: 223.0 (M+H)⁺, (C₉H₇BrN₂).

C. 4-Bromo-1-methyl-2-phenyl-1H-imidazole: To a solution of 4-bromo-2-phenyl-1H-imidazole (0.50 g, 2.24 mmol, 1.00 eq) in THF (10.0 mL) at 0° C., was added NaH (117 mg, 2.91 mmol, 60.0% purity, 1.30 eq) (5 batches) under N₂. The mixture was stirred at 0° C. for 30 min under N₂, then CH₃I (636 mg, 4.48 mmol, 279 μL, 2.00 eq) was dropwise to the mixture. The reaction mixture was stirred at 25° C. for 2 h under N₂. The reaction mixture was poured into 100 mL H₂O under N₂, extracted with ethyl acetate (3×100 mL), washed with brine (50.0 mL), dried over Na₂SO₄ and concentrated to get a residue. The crude product was purified by preparative-TLC (Dichloromethane:Methanol=100:1, R_{f}=0.60) to give the title compound (200 mg, 844 μmol, 37.6% yield). (ESI⁺) m/z: 236.9 (M+H)⁺, (C₁₀H₉BrN₂).

D. 3-(5-(1-Methyl-2-phenyl-1H-imidazol-4-yl)-1-oxoisoindolin-2-yl)piperidine-2,6-dione: To a solution of 3-[1-oxo-5-(4,4,5,5-tetramethyl-1,3,2-dioxaborolan-2-yl)isoindolin-2-yl]piperidine-2,6-dione (131 mg, 354 μmol, 1.20 eq) and 4-bromo-1-methyl-2-phenyl-imidazole (70.0 mg, 295 μmol, 1.00 eq) in dioxane (2.00 mL) and H₂O (0.10 mL) was added K₃PO₄ (125 mg, 590 μmol, 2.00 eq) and Ru-Phos-Pd-G₃ (49.3 mg, 59.0 μmol, 0.20 eq) under N₂. The reaction mixture was stirred at 100° C. for 2 h under N₂. The reaction mixture was concentrated in vacuum to give residue. The residue was purified by preparative-HPLC (using a Welch Xtimate C18 (150 mm×25 mm 10 Nm) and gradient of 3-33% acetonitrile in water containing 0.05% FA over 58 min at a flow rate of 25 mL/min to give the title compound (17.8 mg, 42.1 μmol, 14.2% yield, 94.7% purity in HPLC at 220 nm) as white solid. ¹H NMR: (400 MHz, DMSO-d₆) δ 10.9 (s, 1H), 8.02 (s, 1H), 7.79-7.77 (m, 1H), 7.72-7.69 (m, 3H), 7.54-7.48 (m, 4H), 5.13-5.09 (m, 1H), 4.51-4.33 (m, 2H), 3.80 (s, 3H), 2.91-2.88 (m, 1H), 2.63-2.62 (m, 1H), 2.43-2.40 (m, 1H), 2.03-2.00 (m, 1H). (ESI⁺) m/z: 401.1 (M+H)⁺, (C₂₃H₂₀N₄O₃).

### Example 26

### Synthesis of 3-(5-(1-Ethyl-5-phenyl-1H-imidazol-4-yl)-1-oxoisoindolin-2-yl)piperidine-2,6-dione

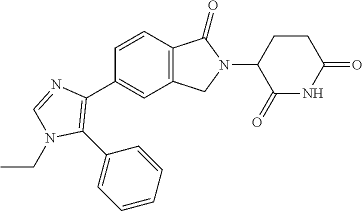 [SMILES: CCn1cnc(-c2ccc3c(c2)CN(C2CCC(=O)NC2=O)C3=O)c1-c1ccccc1]

A. (E)-N-(2-Phenyl-1-tosylvinyl) formamide: To a solution of t-BuOK (6.32 g, 56.3 mmol, 1.10 eq) in THF (80.0 mL) and cooled to −40° C. A solution of 2-tosylacetonitrile (10.0 g, 51.2 mmol, 1.00 eq) in THF (20.0 mL) was added dropwise at −40° C. The mixture was stirred at −40° C. for 0.5 h. A solution of benzaldehyde (5.71 g, 53.8 mmol, 5.44 mL, 1.05 eq) in THF (20.0 mL) was added dropwise at −40° C. Then the mixture was stirred at −40° C. for 0.5 h. The mixture was poured into ice water (100 mL), neutralized with 1 N HCl solution (pH=7) and extracted with DCM (3×100 mL). Then the organic layer was dried by Na₂SO₄, filtered and concentrated to get residue. The residue was triturated with MTBE (2×20.0 mL) at 25° C. to give the title compound (12.0 g, 39.7 mmol, 77.6% yield, 99.8% purity in LCMS at 220 nm) as yellow solid. (ESI⁺) m/z: 302.6 (M+H)⁺, (C₁₆H₁₅NO₃S).

B. 1-Ethyl-5-phenyl-1H-imidazole: To a solution of (E)-N-(2-phenyl-1-tosylvinyl) formamide (2.00 g, 6.63 mmol, 1.00 eq) in DME (20.0 mL) was added TEA (3.35 g, 33.1 mmol, 4.61 mL, 5.00 eq) at −5° C. Then POCl₃ (0.81 g, 5.30 mmol, 492 μL, 0.80 eq) was added dropwise at −5° C. Then the reaction mixture was stirred at 0° C. for 1 h. Then the mixture was added ethylamine (636 mg, 14.1 mmol, 923 μL, 2.00 eq). Then the mixture was stirred at 25° C. for 4 h. The mixture was poured into water (100 mL) at 0° C. and extracted with DCM (3×80.0 mL). Combined organic were dried over Na₂SO₄, filtered and concentrated under reduced pressure to give a residue. The residue was purified by column chromatography (SiO₂, Petroleum ether:Ethyl acetate=10:1, TLC:Dichloromethane:Methanol=10:1, R_{f}=0.2) to give the title compound (400 mg, 2.29 mmol, 35.0% yield, 98.5% purity in LCMS at 220 nm) was obtained as brown oil. (ESI⁺) m/z: 173.0 (M+H)⁺, (C₁₁H₁₂N₂).

C. 4-Bromo-1-ethyl-5-phenyl-1H-imidazole: To a solution of 1-ethyl-5-phenyl-1H-imidazole (386 mg, 2.24 mmol, 1.00 eq) in MeCN (15.0 mL) was added NBS (319 mg, 1.79 mmol, 0.80 eq) under N₂ at 25° C. The mixture was stirred at 25° C. for 1 h under N₂. The reaction mixture was poured into H₂O (10.0 mL) and extracted with dichloromethane (3×10.0 mL). The combined organic layer was washed with brine (3×10.0 mL), dried over Na₂SO₄, filtered and concentrated. The residue was purified by preparative-TLC (Dichloromethane:Methanol=10:1, R_{f}=0.30) to give the title compound (250 mg, 995 μmol, 44.4% yield) as brown oil. ¹H NMR: (400 MHz, DMSO-d₆) δ 7.84-7.82 (m, 1H), 7.51-7.42 (m, 5H), 3.97-3.91 (m, 2H), 1.14-1.10 (m, 3H). (ESI⁺) m/z: 251.1 (M+H)⁺, (C₁₁H₁₁BrN₂).

D. 3-(5-(1-Ethyl-5-phenyl-1H-imidazol-4-yl)-1-oxoisoindolin-2-yl)piperidine-2,6-dione: To a solution of 4-bromo-1-ethyl-5-phenyl-1H-imidazole (200 mg, 796 μmol, 1.00 eq) and 3-(1-oxo-5-(4,4,5,5-tetramethyl-1,3,2-dioxaborolan-2-yl)isoindolin-2-yl)piperidine-2,6-dione (383 mg, 1.04 mmol, 1.30 eq) in dioxane (5.00 mL) and H₂O (0.25 mL) was added Ru-Phos-Pd-G₃ (99.9 mg, 119 μmol, 0.15 eq) and K₃PO₄ (507 mg, 2.39 mmol, 3.00 eq) under N₂ at 25° C. The mixture was stirred at 100° C. for 2 h under N₂. The reaction mixture was poured into H₂O (15.0 mL) and extracted with Ethyl acetate (3×10.0 mL). The combined organic layer was washed with brine (3×10.0 mL), dried over Na₂SO₄, filtered and concentrated. The residue was purified by preparative-HPLC (using a Phenomenex luna C18 (150×25 mm×10 μm) and gradient of 5.00%-35.0% acetonitrile in water containing 0.05% FA over 13 min at a flow rate of 25 mL/min to give the title compound (10.5 mg, 24.9 μmol, 3.14% yield, 98.6% purity in HPLC at 220 nm) was obtained as white solid. ¹H NMR: (400 MHz, DMSO-d₆) δ 10.9 (s, 1H), 7.96-7.86 (m, 1H), 7.61-7.49 (m, 5H), 7.42-7.40 (m, 3H), 5.10-5.02 (m, 1H), 4.29-4.14 (m, 2H), 3.91-3.78 (m, 2H), 2.90-2.86 (m, 1H), 2.60-2.58 (m, 1H), 2.34-2.32 (m, 1H), 1.97-1.96 (m, 1H), 1.17-1.13 (m, 3H). (ESI⁺) m/z: 415.1 (M+H)⁺, (C₂₄H₂₂N₄O₃).

### Example 27

### Synthesis of 3-(5-(1-(difluoromethyl)-5-phenyl-1H-pyrazol-4-yl)-1-oxoisoindolin-2-yl)piperidine-2,6-dione

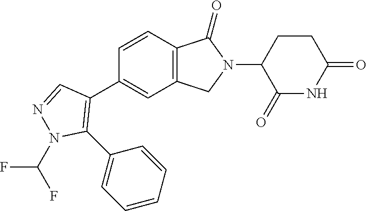 [SMILES: O=C1CCC(N2Cc3cc(-c4cnn(C(F)F)c4-c4ccccc4)ccc3C2=O)C(=O)N1]

A. 4-Bromo-1-(difluoromethyl)-5-phenyl-1H-pyrazole: To a solution of 4-bromo-5-phenyl-1H-pyrazole (1.00 g, 4.48 mmol, 1.00 eq) and 18-Crown-6 (646 mg, 1.79 mmol, 0.40 eq) in ACN (25.0 mL). The reagents were stirred until a colorless solution formed then (2-chloro-2,2-difluoro-acetyl)oxysodium (1.37 g, 8.97 mmol, 2.00 eq) was added to the reaction mixture heated to 80° C. for 48 h. The reaction mixture was filtered through celite, washed with EtOAc (3×10.0 mL). Combined organic layers were concentrated under reduce pressure to give the title compound (0.40 g, 1.26 mmol, 28.0% yield) as white solid. (ESI⁺) m/z: 272.9 (M+H)⁺, (C₁₀H₇BrF₂N₂).

B. 3-(5-(1-(Difluoromethyl)-5-phenyl-1H-pyrazol-4-yl)-1-oxoisoindolin-2-yl)piperidine-2,6-dione: To a solution of 3-[1-oxo-5-(4,4,5,5-tetramethyl-1,3,2-dioxaborolan-2-yl)isoindolin-2-yl]piperidine-2,6-dione (418 mg, 1.13 mmol, 1.20 eq) and 4-bromo-1-(difluoromethyl)-5-phenyl-pyrazole (300 mg, 941 μmol, 1.00 eq) in dioxane (3.00 mL) and H₂O (0.15 mL) was added Ru-Phos-Pd-G₃ (157 mg, 188 μmol, 0.20 eq) and K₃PO₄ (399 mg, 1.88 mmol, 2.00 eq) under N₂. The reaction mixture was stirred at 100° C. for 2 h under N₂. The reaction mixture was concentrated in vacuum to get residue. The residue was purified by preparative-HPLC using a Welch Xtimate C18 (150 mm×25 mm 10 μm) and gradient of 29-59% acetonitrile in water containing 0.05% FA over 58 min at a flow rate of 20 mL/min to give the title compound (122 mg, 278 μmol, 29.6% yield, 99.7% purity in HPLC at 220 nm) as yellow solid. ¹H NMR: (400 MHz, DMSO-d₆) δ 10.9 (s, 1H), 8.60 (s, 1H), 7.90-7.68 (m, 1H), 7.55 (s, 1H), 7.41 (s, 1H), 7.40-7.38 (m, 6H), 5.13-5.08 (m, 1H), 4.45-4.27 (m, 2H), 2.94-2.89 (m, 1H), 2.61-2.50 (m, 1H), 2.42-2.37 (m, 1H), 2.02-2.00 (m, 1H). (ESI⁺) m/z: 437.2 (M+H)⁺, (C₂₃H₁₈F₂N₄O₃).

### Example 28

### Synthesis of 3-(5-(1-methyl-2-phenyl-1H-imidazol-5-yl)-1-oxoisoindolin-2-yl)piperidine-2,6-dione

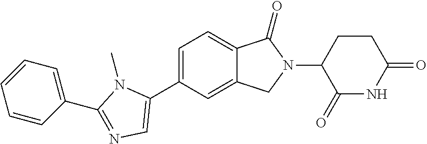 [SMILES: Cn1c(-c2ccc3c(c2)CN(C2CCC(=O)NC2=O)C3=O)cnc1-c1ccccc1]

A. 3-(5-(1-Methyl-2-phenyl-1H-imidazol-5-yl)-1-oxoisoindolin-2-yl)piperidine-2,6-dione: To a solution of 3-[1-oxo-5-(4,4,5,5-tetramethyl-1,3,2-dioxaborolan-2-yl)isoindolin-2-yl]piperidine-2,6-dione (168 mg, 455 μmol, 1.20 eq) and 5-bromo-1-methyl-2-phenyl-imidazole (90.0 mg, 379 μmol, 1.00 eq) in dioxane (1.00 mL) and H₂O (0.05 mL) was added Ru-Phos-Pd-G₃ (63.5 mg, 75.9 μmol, 0.20 eq) and K₃PO₄ (161 mg, 759 μmol, 2.00 eq) under N₂. The reaction mixture was stirred at 100° C. for 2 h under N₂. The reaction mixture was concentrated in vacuum to give residue. The residue was purified by preparative-HPLC (using a Welch Xtimate C18 (150 mm×25 mm 10 μm) and gradient of 1-31% acetonitrile in water containing 0.05% FA over 58 min at a flow rate of 25 mL/min to give the title compound (21.2 mg, 52.4 μmol, 13.8% yield, 99.0% purity in HPLC at 220 nm) as white solid. ¹H NMR: (400 MHz, DMSO-d₆) δ 11.0 (s, 1H), 7.98 (s, 1H), 7.94 (d, J=8.0 Hz, 1H), 7.89 (s, 1H), 7.83-7.82 (m, 2H), 7.78 (d, J=8.0 Hz, 1H), 7.71-7.69 (m, 3H), 5.19-5.14 (m, 1H), 4.60-4.43 (m, 2H), 3.77 (s, 3H), 2.98-2.90 (m, 1H), 2.64-2.50 (m, 1H), 2.47-2.43 (m, 1H), 2.05-2.04 (m, 1H). (ESI⁺) m/z: 401.2 (M+H)⁺, (C₂₃H₂₀N₄O₃).

### Example 29

### Synthesis of 3-(5-(3-Methyl-5-phenylisoxazol-4-yl)-1-oxoisoindolin-2-yl)piperidine-2,6-dione

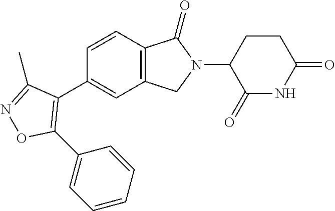 [SMILES: Cc1noc(-c2ccccc2)c1-c1ccc2c(c1)CN(C1CCC(=O)NC1=O)C2=O]

A. 3-(5-(3-Methyl-5-phenylisoxazol-4-yl)-1-oxoisoindolin-2-yl)piperidine-2,6-dione: To a solution of 4-iodo-3-methyl-5-phenylisoxazole (100 mg, 350 μmol, 1.00 eq) and 3-(1-oxo-5-(4,4,5,5-tetramethyl-1,3,2-dioxaborolan-2-yl)isoindolin-2-yl)piperidine-2,6-dione (168 mg, 456 μmol, 1.30 eq) in dioxane (2.00 mL) and H₂O (0.10 mL) was added K₃PO₄ (223 mg, 1.05 mmol, 3.00 eq) and Ru-Phos-Pd-G₃ (58.6 mg, 70.1 μmol, 0.20 eq) under N₂ at 25° C. The mixture was stirred at 100° C. for 4 h under N₂. The reaction mixture was concentrated under reduced pressure to get a residue at 45° C. The residue was purified by preparative-HPLC (using a Phenomenex luna C18 (150×25 mm×10 μm) and gradient of 26.0%-56.0% acetonitrile in water containing 0.05% FA over 13 min at a flow rate of 25 mL/min to give the title compound (17.7 mg, 43.1 μmol, 12.3% yield, 97.5% purity in HPLC at 220 nm) was obtained as white solid. ¹H NMR: (400 MHz, DMSO-d₆) δ 11.03 (s, 1H), 7.66-7.51 (m, 1H), 7.49-7.47 (m, 1H), 7.45-7.43 (m, 6H), 5.39-5.09 (m, 1H), 4.55-4.43 (m, 2H), 2.96-2.89 (m, 1H), 2.62-2.58 (m, 1H), 2.43-2.39 (m, 1H), 2.21 (s, 3H), 2.05-2.03 (m, 1H). (ESI⁺) m/z: 402.0 (M+H)⁺, (C₂₃H₁₉N₃O₄).

### Example 30

### Synthesis of 3-(5-(3-Methyl-5-phenyl-1H-pyrazol-4-yl)-1-oxoisoindolin-2-yl) piperidine-2, 6-dione

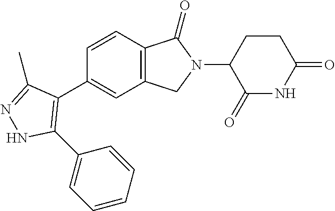 [SMILES: Cc1n[nH]c(-c2ccccc2)c1-c1ccc2c(c1)CN(C1CCC(=O)NC1=O)C2=O]

A. 4-Iodo-3-methyl-5-phenyl-1H-pyrazole: To a solution of 12 (1.28 g, 5.06 mmol, 1.02 mL, 1.00 eq) and (Diacetoxyiodo)benzene (1.63 g, 5.06 mmol, 1.00 eq) in DCM (15.0 mL) was added 3-methyl-5-phenyl-1H-pyrazole (800 mg, 5.06 mmol, 1.00 eq). Then the reaction mixture was stirred at 25° C. for 1 h. The reaction mixture was poured into water (50.0 mL) and extracted with DCM (3×40.0 mL). The combine organic layer was washed with saturated aqueous Na₂SO₃ and dried over by Na₂SO₄, filtered and concentrate filtrate under reduced pressure to get residue. The residue was purified by preparative-HPLC (using a Phenomenex luna C18 (150×25 mm×7 μm) and gradient of 34.0%-64.0% acetonitrile in water containing 0.05% FA over 15 mins at a flow rate of 25 mL/min to give the title compound (548 mg, 1.93 mmol, 38.2% yield, 100% purity in LCMS at 220 nm) was obtained as light yellow solid. ¹H NMR: (400 MHz, CDCl₃) δ 9.08-8.92 (m, 1H), 7.72-7.70 (m, 2H), 7.45-7.27 (m, 3H), 2.21 (s, 3H). (ESI⁺) m/z: 283.9 (M+H)⁺, (C₁₀H₉IN₂).

B. 3-(5-(3-Methyl-5-phenyl-1H-pyrazol-4-yl)-1-oxoisoindolin-2-yl) piperidine-2, 6-dione: To a solution of 4-iodo-3-methyl-5-phenyl-1H-pyrazole (100 mg, 351 μmol, 1.00 eq) and 3-(1-oxo-5-(4,4,5,5-tetramethyl-1,3,2-dioxaborolan-2-yl)isoindolin-2-yl)piperidine-2,6-dione (390 mg, 1.06 mmol, 3.00 eq) in dioxane (4.00 mL) and H₂O (0.20 mL) was added K₃PO₄ (224 mg, 1.06 mmol, 3.00 eq) and Ru-Phos-Pd-G₃ (29.4 mg, 35.2 μmol, 0.10 eq) at 25° C. under N₂. The mixture was stirred at 100° C. for 3 h under N₂. The reaction mixture was concentrated under reduced pressure to get a residue at 45° C. The residue was purified by preparative-HPLC (using a Phenomenex luna C18 (150×25 mm×10 μm) and gradient of 14.0%-44.0% acetonitrile in water containing 0.05% FA over 13 min at a flow rate of 25 mL/min to give the title compound (33.2 mg, 82.4 μmol, 23.4% yield, 99.5% purity in HPLC at 220 nm) was obtained as white solid. ¹H NMR: (400 MHz, DMSO-d₆) δ 13.0-12.9 (m, 1H), 10.9 (s, 1H), 7.68-7.66 (m, 1H), 7.43-7.24 (m, 7H), 5.13-5.09 (m, 1H), 4.46-4.28 (m, 2H), 2.92-2.90 (m, 1H), 2.62-2.61 (m, 1H), 2.40-2.36 (m, 1H), 2.25-2.19 (m, 3H), 2.09-2.02 (m, 1H). (ESI⁺) m/z: 400.1 (M+H)⁺, (C₂₃H₂₀N₄O₃).

### Example 31

### Synthesis of 3-(1-Oxo-5-(4-phenylisothiazol-5-yl) isoindolin-2-yl) piperidine-2, 6-dione

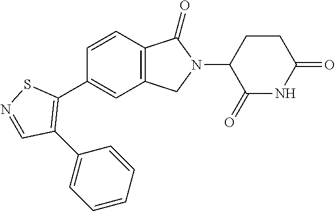 [SMILES: O=C1CCC(N2Cc3cc(-c4sncc4-c4ccccc4)ccc3C2=O)C(=O)N1]

A. 5-Bromo-4-iodoisothiazole: To a solution of 5-bromoisothiazole (1.00 g, 6.10 mmol, 1.00 eq) in TFA (10.0 mL) was added NIS (1.37 g, 6.10 mmol, 1.00 eq) under N₂ at 25° C. The mixture was stirred at 80° C. for 12 h under N₂. The reaction mixture was poured into H₂O (15.0 mL) and extracted with Ethyl acetate (3×10.0 mL). The combined organic layer was washed with brine (3×10.0 mL), dried over Na₂SO₄, filtered and concentrated. The residue was purified by preparative-TLC (Petroleum ether:Ethyl acetate=10:1, R_{f}=0.30) to give the title compound (1.42 g, 4.90 mmol, 80.3% yield, 97.9% purity in LCMS at 220 nm) as light yellow solid. ¹H NMR: (400 MHz, CDCl₃) δ 8.29 (s, 1H). (ESI⁺) m/z: 291.7 (M+H)⁺, (C₃HBrINS).

B. 5-Bromo-4-phenylisothiazole: To a solution of 5-bromo-4-iodoisothiazole (300 mg, 1.03 mmol, 1.00 eq) and phenylboronic acid (113 mg, 931 μmol, 0.90 eq) in dioxane (6.00 mL) and H₂O (0.30 mL) was added Pd(PPh₃)Cl₂ (72.6 mg, 103 μmol, 0.10 eq) and NaHCO₃ (330 mg, 3.93 mmol, 153 μL, 3.80 eq) under N₂ at 25° C. The mixture was stirred at 60° C. for 24 h under N₂. The reaction mixture was poured into H₂O (10.0 mL) and extracted with Dichloromethane (3×10.0 mL). The combined organic layer was washed with brine (3×10.0 mL), dried over Na₂SO₄, filtered and concentrated. The residue was purified by preparative-TLC (Dichloromethane:Methanol=10:1, R_{f}=0.70) to give the title compound (70.0 mg, 291 μmol, 28.1% yield) as light yellow oil. (ESI⁺) m/z: 238.9 (M+H)⁺, (C₉H₆BrNS).

C. 3-(1-Oxo-5-(4-phenylisothiazol-5-yl) isoindolin-2-yl) piperidine-2, 6-dione: To a solution of 3-(1-oxo-5-(4,4,5,5-tetramethyl-1,3,2-dioxaborolan-2-yl)isoindolin-2-yl)piperidine-2,6-dione (501 mg, 1.35 mmol, 2.50 eq) and 5-bromo-4-phenylisothiazole (130 mg, 541 μmol, 1.00 eq) in dioxane (5.00 mL) and H₂O (0.25 mL) was added K₃PO₄ (344 mg, 1.62 mmol, 3.00 eq) and Ru-Phos-Pd-G₃ (45.3 mg, 54.1 μmol, 0.10 eq) under N₂ at 25° C. The mixture was stirred at 100° C. for 4 h under N₂. The reaction mixture was concentrated under reduced pressure to get a residue at 45° C. The residue was purified by preparative-HPLC (using a Phenomenex luna C18 (150×25 mm×10 μm) and gradient of 28.0%-58.0% acetonitrile in water containing 0.05% FA over 13 min at a flow rate of 25 mL/min to give the title compound (30.0 mg, 73.4 μmol, 13.5% yield, 98.7% purity in HPLC at 220 nm) was obtained as white solid. ¹H NMR: (400 MHz, DMSO-d₆) δ 11.0-10.9 (m, 1H), 8.78 (s, 1H), 7.75-7.73 (m, 1H), 7.76 (s, 1H), 7.42-7.35 (m, 6H), 5.14-5.09 (m, 1H), 4.51-4.31 (m, 2H), 2.89-2.83 (m, 1H), 2.64-2.60 (m, 1H), 2.38-2.32 (m, 1H), 2.03-1.99 (m, 1H). (ESI⁺) m/z: 403.9 (M+H)⁺, (C₂₂H₁₇N₃O₃S).

### Example 32

### Synthesis of 3-(1-Oxo-5-(1-phenyl-1H-imidazol-4-yl)isoindolin-2-yl)piperidine-2,6-dione

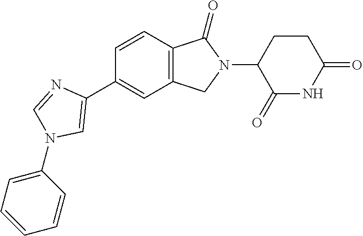 [SMILES: O=C1CCC(N2Cc3cc(-c4cn(-c5ccccc5)cn4)ccc3C2=O)C(=O)N1]

A. 4-Bromo-1-phenyl-1H-imidazole: To a solution of 1-phenyl-1H-imidazole (800 mg, 5.55 mmol, 1.00 eq) in ACN (8.00 mL) was added NBS (790 mg, 4.44 mmol, 0.80 eq) under N₂ at 0° C. The mixture was stirred at 25° C. for 2 h under N₂. The reaction mixture was poured into H₂O (20.0 mL) and extracted with ethyl acetate (3×15.0 mL). The combined organic layer was washed with brine (3×15.0 mL), dried over Na₂SO₄, filtered and concentrated. The mixture was purified by column chromatography (SiO₂, Petroleum ether:Ethyl acetate=10:1 to 0:1, TLC:Petroleum ether:Ethyl acetate=5:1, R_{f}=0.3) to give the title compound (330 mg, 1.48 mmol, 26.6% yield) as yellow solid. ¹H NMR: (400 MHz, CDCl₃) δ 7.73 (s, 1H), 7.52-7.49 (m, 3H), 7.43-7.36 (m, 3H). (ESI⁺) m/z: 224.9 (M+H)⁺, (C₉H₇BrN₂).

B. 3-(1-Oxo-5-(1-phenyl-1H-imidazol-4-yl)isoindolin-2-yl)piperidine-2,6-dione: To a solution of 3-[1-oxo-5-(4,4,5,5-tetramethyl-1,3,2-dioxaborolan-2-yl)isoindolin-2-yl]piperidine-2,6-dione (414 mg, 1.12 mmol, 2.50 eq), 4-bromo-1-phenyl-1H-imidazole (100 mg, 448 μmol, 1.00 eq) and K₃PO₄ (285 mg, 1.34 mmol, 3.00 eq) in dioxane (5.00 mL) and H₂O (0.25 mL) was added Ru-Phos-Pd-G₃ (37.5 mg, 44.8 μmol, 0.10 eq) under N₂ at 25° C. The mixture was stirred at 100° C. for 4 h under N₂. The reaction mixture was concentrated under reduced pressure to get a residue at 45° C. The residue was purified by preparative-HPLC (using a Phenomenex luna C18 (150×25 mm×10 μm) and gradient of 1.00%-31.0% acetonitrile in water containing 0.05% FA over 15 mins at a flow rate of 25 mL/min to give the title compound (51.0 mg, 127 μmol, 28.4% yield, 96.6% purity in HPLC at 220 nm) was obtained as white solid. ¹H NMR: (400 MHz, DMSO-d₆) δ 10.9 (s, 1H), 7.61-7.56 (m, 3H), 7.49-7.47 (m, 3H), 7.34-7.25 (m, 4H), 5.11-5.06 (m, 1H), 4.42-4.23 (m, 2H), 2.92-2.86 (m, 1H), 2.60 (s, 1H), 2.38-2.32 (m, 1H), 2.01-1.97 (m, 1H). (ESI⁺) m/z: 386.1 (M+H)⁺, (C₂₂H₁₈N₄O₃).

### Example 33

### Synthesis of 3-(1-Oxo-5-(5-phenyl-1H-pyrazol-1-yl)isoindolin-2-yl)piperidine-2,6-dione

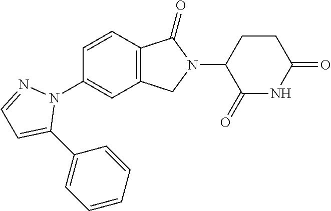 [SMILES: O=C1CCC(N2Cc3cc(-n4nccc4-c4ccccc4)ccc3C2=O)C(=O)N1]

A. 3-(5-Nitro-1-oxoisoindolin-2-yl)piperidine-2,6-dione: To a solution of methyl 2-(bromomethyl)-4-nitro-benzoate (1.00 g, 3.65 mmol, 1.00 eq) and 3-aminopiperidine-2,6-dione (660 mg, 4.01 mmol, 1.10 eq, HCl) in DMF (10.0 mL) was added K₂CO₃ (1.51 g, 10.9 mmol, 3.00 eq). The reaction mixture was stirred at 80° C. for 1 h. The reaction mixture was concentrated to give residue. To the resulting residue, water (10.0 mL) was added and the mixture stirred at 25° C. for 30 min. The resultant solid was filtered, washed with EtOAc (2×10.0 mL) and concentrated under reduced pressure to give the title compound (0.80 g, 2.77 mmol, 75.0% yield, 100% purity in HPLC at 220 nm) as white solid. ¹H NMR: (400 MHz, DMSO-d₆) δ 11.0 (s, 1H), 8.52 (s, 1H), 8.37-8.34 (m, 1H), 7.98 (d, J=8.0 Hz, 1H), 5.18-5.13 (m, 1H), 4.63-4.46 (m, 2H), 2.91-2.88 (m, 1H), 2.63-2.62 (m, 1H), 2.59-2.41 (m, 1H), 2.06-2.04 (m, 1H). (ESI⁺) m/z: 290.0 (M+H)⁺, (C₁₃H₁₁N₃₀₅).

B. 3-(5-Amino-1-oxoisoindolin-2-yl)piperidine-2,6-dione: To a solution of 3-(5-nitro-1-oxo-isoindolin-2-yl)piperidine-2,6-dione (0.80 g, 2.77 mmol, 1.00 eq) in THF (8.00 mL) and MeOH (8.00 mL) was added Pd/C (0.30 g, 10% purity) under N₂. The reaction mixture was stirred at 25° C. for 12 h under H₂ (15 psi. The reaction mixture was filtered by celite, and concentrated under reduced pressure to give the title compound (0.20 g, 771 μmol, 27.8% yield) as white solid. ¹H NMR: (400 MHz, DMSO-d₆) δ 10.9 (s, 1H), 7.34 (d, J=8.8 Hz, 1H), 6.62 (t, J=1.0 Hz, 2H), 5.79 (s, 2H), 5.02-4.97 (m, 1H), 4.26-4.08 (m, 2H), 2.90-2.59 (m, 1H), 2.58-2.54 (m, 1H), 2.50-2.31 (m, 1H), 1.95-1.93 (m, 1H). (ESI⁺) m/z: 260.1 (M+H)⁺, (C₁₃H₁₃N₃O₃).

C. 3-(5-Hydrazineyl-1-oxoisoindolin-2-yl)piperidine-2,6-dione: To a solution of 3-(5-amino-1-oxo-isoindolin-2-yl)piperidine-2,6-dione (50.0 mg, 192 μmol, 1.00 eq) in conc HCl (1.00 mL) was added a solution of NaNO₂ (13.3 mg, 192 μmol, 1.00 eq) in H₂O (0.50 mL) at 0° C. A solution of SnCl₂·2H₂O (87.0 mg, 385 μmol, 2.00 eq) in conc HCl (1.00 mL) was added dropwise after 0.5 h. The reaction mixture was stirred at 25° C. for 12 h. The reaction mixture was concentrated in vacuum to give the title compound (0.19 g, crude) as yellow solid. (ESI⁺) m/z: 275.1 (M+H)⁺, (C₁₃H₁₄N₄O₃).

D. 3-(1-Oxo-5-(5-phenyl-1H-pyrazol-1-yl)isoindolin-2-yl)piperidine-2,6-dione: A solution of 3-(5-hydrazino-1-oxo-isoindolin-2-yl)piperidine-2,6-dione (151 mg, 553 μmol, 1.20 eq) and 3-phenylprop-2-ynal (60.0 mg, 461 μmol, 56.2 μL, 1.00 eq) in ACN (1.00 mL) was stirred at 25° C. for 1 h. Then to the reaction mixture was added Cu(OAc)₂ (8.37 mg, 46.1 μmol, 0.10 eq). The reaction mixture was stirred at 80° C. for 8 h. The reaction mixture was concentrated in vacuum to get residue. The residue was purified by preparative-HPLC using a Welch Xtimate C18 (150 mm×25 mm 5 μm) and gradient of 20-50% acetonitrile in water containing 0.05% FA over 63 min at a flow rate of 20 mL/min to give the title compound (13.4 mg, 32.6 μmol, 7.00% yield, 94.0% purity in HPLC at 220 nm) as yellow solid. ¹H NMR: (400 MHz, DMSO-d₆) δ 10.9 (s, 1H), 7.83 (s, 1H), 7.70 (d, J=8.4 Hz, 1H), 7.62 (s, 1H), 7.39-7.37 (m, 3H), 7.29-7.26 (m, 3H), 6.72 (s, 1H), 5.13-5.08 (m, 1H), 4.48-4.30 (m, 2H), 2.94-2.84 (m, 1H), 2.65-2.60 (m, 1H), 2.43-2.36 (m, 1H), 2.04-1.99 (m, 1H). (ESI⁺) m/z: 387.2 (M+H)⁺, (C₂₂H₁₈N₄O₃).

### Example 34

### Synthesis of 3-(1-Oxo-5-(1-phenyl-1H-pyrazol-3-yl)isoindolin-2-yl)piperidine-2,6-dione

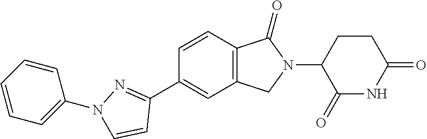 [SMILES: O=C1CCC(N2Cc3cc(-c4ccn(-c5ccccc5)n4)ccc3C2=O)C(=O)N1]

A. 3-(1-Oxo-5-(1-phenyl-1H-pyrazol-3-yl)isoindolin-2-yl)piperidine-2,6-dione: To a solution of 1-phenyl-3-(4,4,5,5-tetramethyl-1,3,2-dioxaborolan-2-yl)-1H-pyrazole (100 mg, 370 μmol, 1.00 eq), 3-(5-bromo-1-oxoisoindolin-2-yl)piperidine-2,6-dione (179 mg, 555 μmol, 1.50 eq) and K₃PO₄ (235 mg, 1.11 mmol, 3.00 eq) in dioxane (5.00 mL) and H₂O (0.25 mL) was added Ru-Phos-Pd-G₃ (30.9 mg, 37.0 μmol, 0.100 eq) under N₂. The mixture was stirred at 70° C. for 2 h under N₂. The mixture was filtered to collect liquid and the liquid was concentrated under reduced pressure to get residue. The residue was purified by preparative-HPLC (using a column: Welch Xtimate C18 (150×25 mm×5 μm) and gradient of 30.0%-60.0% acetonitrile in water containing 0.05% TFA over 15 min at a flow rate of 25 mL/min to give the title compound (15.7 mg, 39.5 μmol, 11.0% yield, 97.2% purity in HPLC at 220 nm) as white solid. ¹H NMR: (400 MHz, DMSO-d₆) δ 11.04 (s, 1H), 8.68-8.67 (m, 1H), 8.20-8.16 (m, 1H), 8.15-8.14 (m, 1H), 8.14-8.13 (m, 2H), 8.00-7.98 (m, 1H), 7.86-7.84 (m, 1H), 7.41-7.40 (m, 1H), 7.37-7.23 (m, 1H), 7.22 (s, 1H), 5.20-5.14 (m, 1H), 4.48-4.46 (m, 1H), 4.45-4.42 (m, 1H), 2.94-2.92 (m, 1H), 2.58-2.54 (m, 1H), 2.49-2.48 (m, 1H), 2.11-2.06 (m, 1H). (ESI⁺) m/z: 386.4 (M+H)⁺, (C₂₂H₁₈N₄O₃).

### Example 35

### Synthesis of 3-(5-(1-Methyl-3-phenyl-1H-pyrazol-5-yl)-1-oxoisoindolin-2-yl)piperidine-2,6-dione

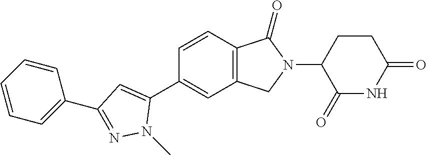 [SMILES: Cn1nc(-c2ccccc2)cc1-c1ccc2c(c1)CN(C1CCC(=O)NC1=O)C2=O]

A. 3-(5-(1-Methyl-3-phenyl-1H-pyrazol-5-yl)-1-oxoisoindolin-2-yl)piperidine-2,6-dione: To solution of 5-bromo-1-methyl-3-phenyl-1H-pyrazole (100 mg, 421 μmol, 1.00 eq), 3-(1-oxo-5-(4,4,5,5-tetramethyl-1,3,2-dioxaborolan-2-yl)isoindolin-2-yl)piperidine-2,6-dione (312 mg, 843 μmol, 2.00 eq) and K₃PO₄ (268 mg, 1.27 mmol, 3.00 eq) in dioxane (5.00 mL) and H₂O (0.25 mL) was added Ru-Phos-Pd-G₃ (35.3 mg, 42.2 μmol, 0.10 eq) under N₂.

The mixture was stirred at 100° C. for 2 h under N₂. The mixture was filtered to collect liquid and the liquid was concentrated under reduced pressure to get residue. The residue was purified by preparative-HPLC using a column: Welch Xtimate C18 (150×25 mm×5 μm) and gradient of 27%-57.0% acetonitrile in water containing 0.05% TFA over 15 min at a flow rate of 25 mL/min to give the title compound (14.4 mg, 34.4 μmol, 8.40% yield, 95.6% purity in HPLC at 220 nm) as an off-white solid. ¹H NMR: (400 MHz, DMSO-d₆) δ 11.01 (s, 1H), 7.88-7.84 (m, 4H), 7.76-7.74 (m, 1H), 7.44-7.40 (m, 2H), 7.33-7.30 (m, 1H), 7.00 (s, 1H), 5.18-5.13 (m, 1H), 4.57-4.53 (m, 1H), 4.45-4.40 (m, 1H), 3.99-3.95 (m, 3H), 2.98-2.92 (m, 1H), 2.56-2.53 (m, 1H), 2.46-2.42 (m, 1H), 2.07-2.04 (m, 1H). (ESI⁺) m/z: 400.4 (M+H)⁺, (C₂₃H₂₀N₄O₃).

### Example 36

### Synthesis of 3-(5-(1-Methyl-5-phenyl-1H-pyrazol-3-yl)-1-oxoisoindolin-2-yl) piperidine-2, 6-dione

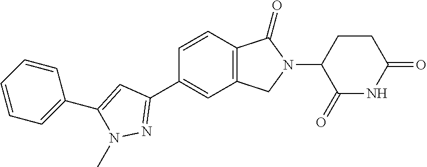 [SMILES: Cn1nc(-c2ccc3c(c2)CN(C2CCC(=O)NC2=O)C3=O)cc1-c1ccccc1]

A. 3-(5-(1-Methyl-5-phenyl-1H-pyrazol-3-yl)-1-oxoisoindolin-2-yl) piperidine-2, 6-dione: To a solution of 3-bromo-1-methyl-5-phenyl-1H-pyrazole (100 mg, 421 μmol, 1.00 eq), 3-(1-oxo-5-(4,4,5,5-tetramethyl-1,3,2-dioxaborolan-2-yl)isoindolin-2-yl)piperidine-2,6-dione (546 mg, 1.48 mmol, 3.50 eq) and K₃PO₄ (268 mg, 1.27 mmol, 3.00 eq) in dioxane (5.00 mL) and H₂O (0.25 mL) was added Ru-Phos-Pd-G₃ (52.9 mg, 63.2 μmol, 0.15 eq) at 25° C. under N₂. The mixture was stirred at 100° C. for 7 h under N₂. The reaction mixture was concentrated under reduced pressure to get a residue at 45° C. The residue was purified by preparative-HPLC using a Phenomenex luna C18 (150×25 mm×10 μm) and gradient of 26.0%-56.0% acetonitrile in water containing 0.05% FA over 13 mins at a flow rate of 25 mL/min to give the title compound (51.3 mg, 125 μmol, 29.6% yield, 97.6% purity in HPLC at 220 nm) was obtained as white solid. ¹H NMR: (400 MHz, DMSO-d₆) δ 11.0 (s, 1H), 8.77-8.00 (m, 2H), 7.77-7.75 (m, 1H), 7.60-7.48 (m, 5H), 7.02 (s, 1H), 5.15-5.10 (m, 1H), 4.52-4.36 (m, 2H), 3.93 (s, 3H), 2.95-2.89 (m, 1H), 2.63-2.62 (m, 1H), 2.44-2.39 (m, 1H), 2.06-1.96 (m, 1H). (ESI⁺) m/z: 400.9 (M+H)⁺, (C₂₃H₂₀N₄O₃).

### Example 37

### Synthesis of 3-(1-Oxo-5-(2-phenyloxazol-4-yl)isoindolin-2-yl)piperidine-2,6-dione

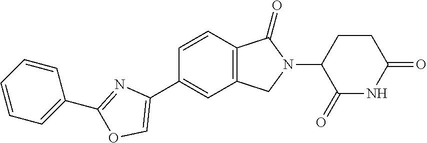 [SMILES: O=C1CCC(N2Cc3cc(-c4coc(-c5ccccc5)n4)ccc3C2=O)C(=O)N1]

A. 3-(1-Oxo-5-(2-phenyloxazol-4-yl)isoindolin-2-yl)piperidine-2,6-dione: To a solution of 4-bromo-2-phenyloxazole (100 mg, 446 μmol, 1.00 eq), 3-(1-oxo-5-(4,4,5,5-tetramethyl-1,3,2-dioxaborolan-2-yl)isoindolin-2-yl)piperidine-2,6-dione (495 mg, 1.34 mmol, 3.00 eq) and K₃PO₄ (284 mg, 1.34 mmol, 3.00 eq) in dioxane (5.00 mL) and H₂O (0.25 mL) was added Ru-Phos-Pd-G₃ (37.3 mg, 44.6 μmol, 0.10 eq) at 25° C. under N₂. The mixture was stirred at 100° C. for 5 h under N₂. The reaction mixture was concentrated under reduced pressure to get a residue at 45° C. The residue was purified by prep-HPLC (using a Phenomenex luna C18 (150×25 mm×10 μm) and gradient of 30.0%-60.0% acetonitrile in water containing 0.05% FA over 13 mins at a flow rate of 25 mL/min to give the title compound (50.4 mg, 133 μmol, 29.9% yield, 99.1% purity in HPLC at 220 nm) was obtained as white solid. ¹H NMR: (400 MHz, DMSO-d₆) δ 11.0 (s, 1H), 8.89-8.88 (m, 1H), 8.12-8.02 (m, 4H), 7.83-7.81 (m, 1H), 7.59-7.58 (m, 3H), 5.16-5.11 (m, 1H), 4.57-4.39 (m, 2H), 2.96-2.88 (m, 1H), 2.67-2.66 (m, 1H), 2.33-2.32 (m, 1H), 2.03-2.01 (m, 1H). (ESI⁺) m/z: 388.1 (M+H)⁺, (C₂₂H₁₇N₃O₄).

### Example 38

### Synthesis of 3-(1-Oxo-5-(3-(pyridin-4-yl)-1H-pyrazol-4-yl)isoindolin-2-yl)piperidine-2,6-dione

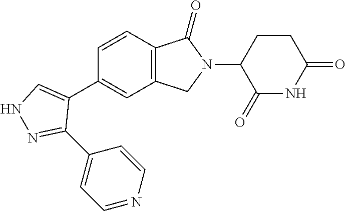 [SMILES: O=C1CCC(N2Cc3cc(-c4c[nH]nc4-c4ccncc4)ccc3C2=O)C(=O)N1]

A. 3-(1-Oxo-5-(3-(pyridin-4-yl)-1H-pyrazol-4-yl) isoindolin-2-yl) piperidine-2,6-dione: To a solution of 4-(4-bromo-1H-pyrazol-3-yl)pyridine (100 mg, 446 μmol, 1.00 eq), 3-(1-oxo-5-(4,4,5,5-tetramethyl-1,3,2-dioxaborolan-2-yl)isoindolin-2-yl)piperidine-2,6-dione (330 mg, 892 μmol, 2.00 eq) and K₃PO₄ (284 mg, 1.34 mmol, 3.00 eq) in dioxane (5.00 mL) and H₂O (0.25 mL) was added cataCXium A Pd G₃ (65.0 mg, 89.2 μmol, 0.20 eq) under N₂.

The mixture was stirred at 100° C. for 18 h under N₂. The reaction mixture was concentrated under reduced pressure to get a residue at 45° C. The residue was purified by preparative-HPLC (using a Phenomenex luna C18 (150×25 mm×10 Nm) and gradient of 0.00%-24.0% acetonitrile in water containing 0.05% FA over 15 min at a flow rate of 25 mL/min) to give the title compound (13.8 mg, 34.1 μmol, 7.63% yield, 95.7% purity in HPLC at 220 nm) was obtained as an off-white solid. ¹H NMR: (400 MHz, DMSO) δ 13.5-13.4 (m, 1H), 11.0 (s, 1H), 8.67-8.53 (m, 2H), 8.15-8.10 (m, 1H), 7.71-7.69 (m, 1H), 7.53 (s, 1H), 7.40-7.39 (m, 3H), 5.13-5.09 (m, 1H), 4.46-4.29 (m, 2H), 2.49-2.87 (m, 1H), 2.66-2.62 (m, 1H), 2.41-2.37 (m, 1H), 2.02-2.01 (m, 1H). (ESI⁺) m/z: 387.9 (M+H)⁺, (C₂₁H₁₇N₅O₃).

### Example 39

### Synthesis of 3-(1-Oxo-5-(5-phenylisoxazol-4-yl)isoindolin-2-yl)piperidine-2,6-dione

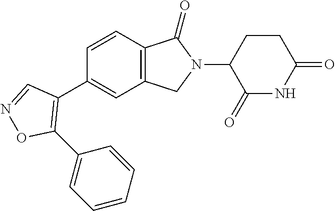 [SMILES: O=C1CCC(N2Cc3cc(-c4cnoc4-c4ccccc4)ccc3C2=O)C(=O)N1]

A. 4-Bromo-5-phenylisoxazole: To a solution of 5-phenylisoxazole (150 mg, 1.03 mmol, 1.00 eq) and NBS (183 mg, 1.03 mmol, 1.00 eq) in acetic acid (2.00 mL). The mixture was stirred at 50° C. for 16 h. The reaction mixture was poured into H₂O (10.0 mL) and extracted with Ethyl acetate (3×10.0 mL). The combined organic layer was washed with brine (3×10.0 mL), and dried over by Na₂SO₄, filtered and the filtrate was concentrated under reduced pressure to get residue. The residue was purified by preparative-TLC (Petroleum ether:Ethyl acetate=5:1, R_{f}=0.80) to give the title compound (130 mg, 393 μmol, 79.5% yield, 88.0% purity in LCMS at 220 nm) as light yellow oil. ¹H NMR: (400 MHz, CDCl₃) δ 8.30 (s, 1H), 8.06-8.03 (m, 2H), 7.53-7.27 (m, 3H). (ESI⁺) m/z: 223.7 (M+H)⁺, (C₉H₆BrNO).

B. 3-(1-Oxo-5-(5-phenylisoxazol-4-yl)isoindolin-2-yl)piperidine-2,6-dione: To a solution of 4-bromo-5-phenylisoxazole (175 mg, 781 μmol, 1.00 eq), 3-(1-oxo-5-(4,4,5,5-tetramethyl-1,3,2-dioxaborolan-2-yl)isoindolin-2-yl)piperidine-2,6-dione (722 mg, 1.95 mmol, 2.50 eq) and K₃PO₄ (497 mg, 2.34 mmol, 3.00 eq) in dioxane (6.00 mL) and H₂O (0.30 mL) was added Ru-Phos-Pd-G₃ (65.3 mg, 78.1 μmol, 0.10 eq) under N₂. The mixture was stirred at 100° C. for 3 h under N₂. The reaction mixture was concentrated under reduced pressure to get a residue at 45° C. The residue was purified by preparative-HPLC (using a Phenomenex luna C18 (150×25 mm×5 μm) and gradient of 28.0%-48.0% acetonitrile in water containing 0.05% FA over 10 min at a flow rate of 25 mL/min) to give the title compound (22.5 mg, 57.2 μmol, 7.34% yield, 98.5% purity in HPLC at 220 nm) as an off-white solid. ¹H NMR: (400 MHz, DMSO-d₆) δ 11.0-10.9 (m, 1H), 9.01-8.98 (m, 1H), 7.78-7.70 (m, 1H), 7.60-7.59 (m, 1H), 7.53-7.50 (m, 2H), 7.45-7.31 (m, 4H), 5.15-5.10 (m, 1H), 4.51-4.32 (m, 2H), 2.95-2.88 (m, 1H), 2.69-2.62 (m, 1H), 2.43-2.38 (m, 1H), 2.03-2.00 (m, 1H). (ESI⁺) m/z: 387.9 (M+H)⁺, (C₂₂H₁₇N₃O₄).

### Example 40

### Synthesis of 3-(5-(1-Methyl-5-phenyl-1H-imidazol-2-yl)-1-oxoisoindolin-2-yl)piperidine-2,6-dione

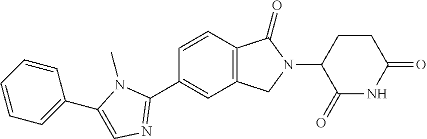 [SMILES: Cn1c(-c2ccccc2)cnc1-c1ccc2c(c1)CN(C1CCC(=O)NC1=O)C2=O]

A. 2-Bromo-1-methyl-5-phenyl-1H-imidazole: To a solution of phenylboronic acid (213 mg, 1.75 mmol, 1.40 eq), 2,5-dibromo-1-methyl-1H-imidazole (300 mg, 1.25 mmol, 1.00 eq) and K₃PO₄ (796 mg, 3.75 mmol, 3.00 eq) in THF (10.0 mL) was added Pd(OAc)₂ (28.0 mg, 125 μmol, 0.10 eq). The mixture was stirred at 70° C. for 16 h. The reaction mixture was poured into H₂O (15.0 mL) and extracted with Ethyl acetate (3×15.0 mL). The combined organic layer was washed with brine (3×15.0 mL), and dried over by Na₂SO₄, filtered and concentrate filtrate under reduced pressure to get residue. The residue was purified by preparative-TLC (Petroleum ether:Ethyl acetate=3:1, R_{f}=0.30) to give the title compound (75.0 mg, 309 μmol, 24.7% yield, 97.7% purity in LCMS at 220 nm) as light yellow solid. ¹H NMR: (400 MHz, CDCl₃) δ 7.46-7.27 (m, 5H), 7.05 (s, 1H), 3.63-3.56 (m, 3H). (ESI⁺) m/z: 236.8 (M+H)⁺, (C₁₀H₉BrN₂).

B. 3-(5-(1-Methyl-5-phenyl-1H-imidazol-2-yl)-1-oxoisoindolin-2-yl)piperidine-2,6-dione: To a solution of 2-bromo-1-methyl-5-phenyl-1H-imidazole (65.0 mg, 274 μmol, 1.00 eq), 3-(1-oxo-5-(4,4,5,5-tetramethyl-1,3,2-dioxaborolan-2-yl)isoindolin-2-yl)piperidine-2,6-dione (253 mg, 685 μmol, 2.50 eq) and K₃PO₄ (174 mg, 822 μmol, 3.00 eq) in dioxane (4.00 mL) and H₂O (0.20 mL) was added Ru-Phos-Pd-G₃ (22.9 mg, 27.4 μmol, 0.10 eq) under N₂. The mixture was stirred at 100° C. for 3 h under N₂. The reaction mixture was concentrated under reduced pressure to get a residue at 45° C. The residue was purified by preparative-HPLC (using a Phenomenex luna C18 (150×25 mm×5 μm) and gradient of 0.00%-30.0% acetonitrile in water containing 0.05% FA over 10 min at a flow rate of 25 mL/min) to give the title compound (7.85 mg, 18.6 μmol, 6.79% yield, 95.0% purity in HPLC at 220 nm) as an off-white solid. ¹H NMR: (400 MHz, DMSO-d₆) δ 11.0 (s, 1H), 8.13-8.12 (m, 1H), 7.97-7.84 (m, 2H), 7.57-7.56 (m, 2H), 7.53-7.50 (m, 2H), 7.44-7.42 (m, 1H), 7.23 (s, 1H), 5.18-5.13 (m, 1H), 4.58-4.41 (m, 2H), 3.73 (s, 3H), 2.96-2.93 (m, 1H), 2.64-2.59 (m, 1H), 2.44-2.41 (m, 1H), 2.05-2.02 (m, 1H). (ESI⁺) m/z: 401.1 (M+H)⁺, (C₂₃H₂₀N₄O₃).

### Example 41

### Synthesis of 3-(5-(1-Methyl-4-phenyl-1H-imidazol-2-yl)-1-oxoisoindolin-2-yl)piperidine-2,6-dione

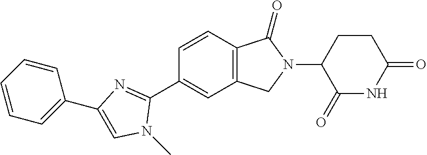 [SMILES: Cn1cc(-c2ccccc2)nc1-c1ccc2c(c1)CN(C1CCC(=O)NC1=O)C2=O]

A. 1-Methyl-4-phenyl-1H-imidazole: To a solution of 4-phenyl-1H-imidazole (2.00 g, 13.8 mmol, 1.00 eq), Cs₂CO₃ (6.78 g, 20.8 mmol, 1.50 eq) in DMF (30.0 mL) was added Mel (3.94 g, 27.7 mmol, 1.73 mL, 2.00 eq) at 20° C. The mixture was stirred at 25° C. for 8 h under N₂. The mixture was poured into H₂O (70.0 mL), extracted with Ethyl acetate (3×40.0 mL). The organic layer was washed with saturated NaCl aqueous (3×30.0 mL), dried over Na₂SO₄, and filtered to collect filtered liquid. The liquid was concentrated under reduced pressure to get residue. The residue was purified by preparative-TLC (Dichloromethane:Methanol=10:1 R_{f}=0.30) to get the title compound (270 mg, 1.69 mmol, 12.3% yield, 99.1% purity in LCMS at 220 nm) as yellow solid. ¹H NMR (400 MHz, DMSO-d₆): δ 7.73-7.71 (m, 2H), 7.61-7.59 (m, 1H), 7.58-7.52 (m, 1H), 7.35-7.31 (m, 2H), 7.19-7.15 (m, 1H), 3.67 (s, 3H). (ESI⁺) m/z: 159.0 (M+H)⁺, (C₁₀H₁₀N₂).

B. 2-Iodo-1-methyl-4-phenyl-1H-imidazole: To a solution of 1-methyl-4-phenyl-1H-imidazole (150 mg, 948 μmol, 1.00 eq) in THF (1.00 mL), n-BuLi (2.50 M, 758 μL, 2.00 eq) was added at −70° C. under N₂ atmosphere and stirred at −70° C. for 1 h. Then 12 (481 mg, 1.90 mmol, 382 μL, 2.00 eq) in THF (3.00 mL) was added from a syringe, keeping the reaction temperature under −50° C. Then the reaction was allowed to 0° C. and stirred for 1 h under N₂. The reaction mixture was poured into saturated NH₄Cl aqueous (30.0 mL) and extracted with ethyl acetate (3×20.0 mL). The combined organic layers were washed with Na₂SO₃ (10.0%, 3×30.0 mL), dried over Na₂SO₄, and filtered to collect liquid, the liquid was concentrated under reduced pressure to the title compound was given (188 mg, 633 μmol, 66.7% yield, 95.7% purity in LCMS at 220 nm) as yellow solid. ¹H NMR (400 MHz, DMSO-d₆): δ 7.81 (s, 1H), 7.68-7.66 (m, 2H), 7.36-7.32 (m, 2H), 7.21-7.17 (m, 1H), 3.60 (s, 3H). (ESI⁺) m/z: 284.8 (M+H)⁺, (C₁₀H₉IN₂).

C. 3-(5-(1-Methyl-4-phenyl-1H-imidazol-2-yl)-1-oxoisoindolin-2-yl)piperidine-2,6-dione: To a solution of 2-iodo-1-methyl-4-phenyl-1H-imidazole (150 mg, 528 μmol, 1.00 eq), 3-(1-oxo-5-(4,4,5,5-tetramethyl-1,3,2-dioxaborolan-2-yl)isoindolin-2-yl)piperidine-2,6-dione (489 mg, 1.32 mmol, 2.50 eq) and K₃PO₄ (336 mg, 1.58 mmol, 3.00 eq) in dioxane (2.50 mL) and H₂O (0.12 mL) was added Ru-Phos-Pd-G₃ (44.2 mg, 52.8 μmol, 0.10 eq) under N₂. The mixture was stirred at 100° C. for 3 h under N₂. Then the reaction mixture was filtered to collect filtered liquid and the liquid was concentrated under reduced pressure to get residue and the residue was purified by preparative-HPLC (using a Phenomenex luna C18 (150×2 5 mm×5 μm) and gradient of 4-34% acetonitrile in water containing 0.5% TFA over 15 min at a flow rate of 25 mL/min to give the title compound (37.4 mg, 91.9 μmol, 17.4% yield, 98.4% purity in HPLC at 220 nm) as white solid. ¹H NMR (400 MHz, DMSO-d₆): δ 11.0 (s, 1H), 8.08-7.99 (m, 2H), 7.98-7.94 (m, 2H), 7.84-7.82 (m, 2H), 7.50-7.46 (m, 2H), 7.38-7.36 (m, 1H), 5.20-5.15 (m, 1H), 4.61-4.55 (m, 1H), 4.49-4.44 (m, 1H), 3.88 (s, 3H), 2.97-2.89 (m, 1H), 2.61-2.60 (m, 1H), 2.44-2.43 (m, 1H), 2.07-2.04 (m, 1H). (ESI⁺) m/z: 401.0 (M+H)⁺, (C₂₃H₂₀N₄O₃).

### Example 42

### Synthesis of 3-(1-Oxo-5-(4-phenyloxazol-2-yl)isoindolin-2-yl)piperidine-2,6-dione

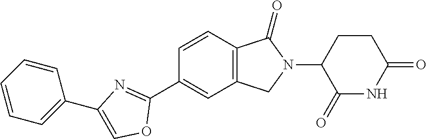 [SMILES: O=C1CCC(N2Cc3cc(-c4nc(-c5ccccc5)co4)ccc3C2=O)C(=O)N1]

A. 4-Phenyloxazole: A mixture of 2-bromo-1-phenyl-ethanone (10.0 g, 50.2 mmol, 1.00 eq) and HCOONH₄ (11.0 g, 175 mmol, 3.50 eq) in HCOOH (50.0 mL) was stirred at 100° C. for 5 h. After the reaction was completed, the reaction mixture was diluted with 100 mL of water and basified to pH=9 with saturated Na₂CO₃ solution. The mixture was extracted with EtOAc (3×100 mL). The combined organic layers were washed with brine (100 mL), dried over anhydrous Na₂SO₄, filtered and the filtrate was concentrated in vacuum to get a residue. The residue was purified by column chromatography (SiO₂, Petroleum ether:EtOAc=100:1 to 5:1, R_{f}=0.60 (Petroleum ether:EtOAc=5:1)) to give the title compound (2.00 g, 12.9 mmol, 25.7% yield, 93.7% purity in LCMS at 220 nm) as yellow oil. (ESI⁺) m/z: 146.3 (M+H)⁺, (C₉H₇NO).

B. 2-Bromo-4-phenyloxazole: To a solution of 4-phenyloxazole (200 mg, 1.29 mmol, 1.00 eq) in THF (2.00 mL) was added dropwise n-BuLi (2.50 M, 568 μL, 1.10 eq) at −78° C. under N₂. The reaction mixture was stirred for additional 0.5 h, then 1,2-dibromo-1,1,2,2-tetrafluoro-ethane (352 mg, 1.36 mmol, 1.05 eq) was added dropwise at −78° C. Then the reaction mixture was stirred at 25° C. for 12 h. After the reaction was completed, the reaction mixture was quenched with 10.0 mL of saturated NH₄Cl solution at 0° C. under N₂. Then the mixture was extracted with EtOAc (3×10.0 mL). The combined organic layers were washed with brine (10.0 mL), dried over Na₂SO₄, filtered and the filtrate was concentrated in vacuum to get a residue. The residue was purified by preparative-TLC (SiO₂, Petroleum ether:EtOAc=5:1, R_{f}=0.80) to give the title compound (150 mg, 669 μmol, 51.8% yield, 100% purity in LCMS at 220 nm) as a white solid. ¹H NMR: (400 MHz, DMSO-d₆) δ 8.78 (s, 1H), 7.76-7.72 (m, 2H), 7.47-7.42 (m, 2H), 7.39-7.34 (m, 1H). (ESI⁺) m/z: 223.8 (M+H)⁺, (C₉H₆BrNO).

C. 3-(1-Oxo-5-(4-phenyloxazol-2-yl)isoindolin-2-yl)piperidine-2,6-dione: To a solution of 2-bromo-4-phenyl-oxazole (120 mg, 535 μmol, 1.00 eq) and 3-[1-oxo-5-(4,4,5,5-tetramethyl-1,3,2-dioxaborolan-2-yl)isoindolin-2-yl]piperidine-2,6-dione (396 mg, 1.07 mmol, 2.00 eq) in dioxane (6.00 mL) and H₂O (0.30 mL) was added Ru-Phos-Pd-G₃ (44.7 mg, 53.5 μmol, 0.10 eq) and K₃PO₄ (341 mg, 1.61 mmol, 3.00 eq). The mixture was stirred at 100° C. for 2 h under N₂. After the reaction was completed, the reaction mixture was filtered and the filtrate was concentrated in vacuum to get a residue. The residue was purified by preparative-HPLC (using a Phenomenex luna C18 (150 mm×25 mm×10 μm) and gradient of 32-62% acetonitrile in water containing 0.5% FA over 8 min at a flow rate of 25 mL/min) to give the title compound (16.2 mg, 41.8 μmol, 7.81% yield, 100% purity in HPLC at 220 nm) as a gray solid. ¹H NMR: (400 MHz, DMSO-d₆) δ 11.0 (s, 1H), 8.82 (s, 1H), 8.31 (s, 1H), 8.21 (dd, J=7.6, 0.8 Hz, 1H), 7.93-7.88 (m, 3H), 7.51-7.46 (m, 2H), 7.41-7.35 (m, 1H), 5.16 (dd, J=13.2, 5.2 Hz, 1H), 4.52 (dd, J=48.8, 17.6 Hz, 2H), 2.97-2.88 (m, 1H), 2.64-2.60 (m, 1H), 2.45-2.38 (m, 1H), 2.09-2.01 (m, 1H). (ESI⁺) m/z: 388.0 (M+H)⁺, (C₂₂H₁₇N₃O₄).

### Example 43

### Synthesis of 3-(1-Oxo-5-(3-phenylisoxazol-5-yl)isoindolin-2-yl)piperidine-2,6-dione

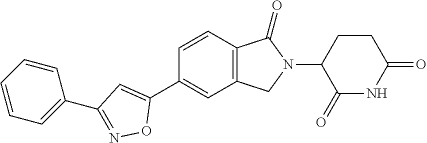 [SMILES: O=C1CCC(N2Cc3cc(-c4cc(-c5ccccc5)no4)ccc3C2=O)C(=O)N1]

A. (2,2-Dibromocyclopropyl)benzene: To a solution of styrene (5.00 g, 48.0 mmol, 1.00 eq) in DCM (25.0 mL) was added TEBA (349 mg, 1.54 mmol, 0.032 eq) and KOH (4.04 g, 72.0 mmol, 1.50 eq) at 40° C. under N₂. The mixture was added CHBr₃ (15.5 g, 61.4 mmol, 5.38 mL, 1.28 eq) at 40° C. under N₂ over 2 h. The mixture was stirred at 25° C. for 20 h under N₂. After the reaction was completed, the reaction mixture was filtered and the filtrate was concentrated under vacuum to give a residue. The residue was purified by column chromatography (SiO₂, Ethyl acetate:Petroleum ether=0:1, R_{f}=0.50 (Ethyl acetate:Petroleum ether=0:1)) to give the title compound (5.00 g, 18.1 mmol, 37.7% yield) as yellow oil. ¹H NMR: (400 MHz, DMSO-d₆) δ 7.38-7.32 (m, 3H), 7.26 (d, J=6.4 Hz, 2H), 2.96 (t, J=8.8 Hz, 1H), 2.13 (t, J=7.6 Hz, 1H), 2.01 (t, J=8.0 Hz, 1H). (ESI⁺) m/z: 274.9 (M+H)⁺, (C₉H₈Br₂).

B. 5-Bromo-3-phenylisoxazole: To a solution of nitridooxonium tetrafluoroborate (2.54 g, 21.7 mmol, 1.20 eq) in dry ACN (15.0 mL) was added dropwise a solution of (2,2-dibromocyclopropyl)benzene (5.00 g, 18.1 mmol, 1.00 eq) in dry ACN (10.0 mL) at 25° C. under N₂. Then the reaction mixture was stirred at 25° C. for 5 h. After the reaction was completed, the reaction mixture was quenched with 30.0 mL of water and extracted with ethyl acetate (3×20.0 mL). The combined organic layers were washed with brine (20.0 mL), dried over anhydrous Na₂SO₄, filtered and the filtrate concentrated in vacuum to get a crude product. The crude product was purified by column chromatography (SiO₂, Petroleum ether: Ethyl acetate=100:1 to 10:1, R_{f}=0.20 (Petroleum ether:Ethyl acetate=10:1)) to give the title compound (500 mg, 1.94 mmol, 12.3% yield, 86.9% purity in LCMS at 220 nm) as yellow oil. ¹H NMR: (400 MHz, DMSO-d₆) δ 7.88-7.84 (m, 2H), 7.56-7.51 (m, 3H), 7.39 (s, 1H). (ESI⁺) m/z: 223.9 (M+H)⁺, (C₉H₆BrNO).

C. 3-(1-Oxo-5-(3-phenylisoxazol-5-yl)isoindolin-2-yl)piperidine-2,6-dione: To a solution of 5-bromo-3-phenyl-isoxazole (100 mg, 446 μmol, 1.00 eq) in dioxane (1.00 mL) and H₂O (0.05 mL) was added 3-[1-oxo-5-(4,4,5,5-tetramethyl-1,3,2-dioxaborolan-2-yl)isoindolin-2-yl]piperidine-2,6-dione (330 mg, 892 μmol, 2.00 eq), K₃PO₄ (189 mg, 892 μmol, 2.00 eq) and Ru-Phos-Pd-G₃ (37.3 mg, 44.6 μmol, 0.10 eq) under N₂. The reaction mixture was stirred at 80° C. for 2 h under N₂. After the reaction was completed, the reaction mixture was filtered and the filtrate was concentrated under vacuum to give a residue. The residue was purified by preparative-HPLC (using a Phenomenex Luna C18 (150 mm×25 mm×10 μm) and gradient of 5-35% acetonitrile in water containing 0.5% TFA over 10 min at a flow rate of 25 mL/min) to give the title compound (19.8 mg, 50.5 μmol, 11.3% yield, 98.8% purity in HPLC at 220 nm) as a white solid. ¹H NMR: (400 MHz, DMSO-d₆) δ 11.03 (s, 1H), 8.18 (s, 1H), 8.08 (d, J=8.4 Hz, 1H), 7.96-7.91 (m, 3H), 7.80 (s, 1H), 7.61-7.54 (m, 3H), 5.16 (dd, J=13.2, 4.8 Hz, 1H), 4.53 (dd, J=52.4, 17.6 Hz, 2H), 2.98-2.88 (m, 1H), 2.67 (s, 1H), 2.33 (s, 1H), 2.07 (s, 1H). (ESI⁺) m/z: 388.1 (M+H)⁺, (C₂₂H₁₇N₃O₄).

### Example 44

### Synthesis of 3-(5-(2-Cyclopropyl-1-methyl-5-(pyridin-3-yl)-1H-imidazol-4-yl)-1-oxoisoindolin-2-yl)piperidine-2,6-dione

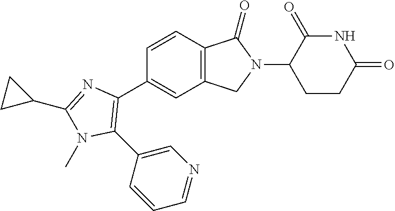 [SMILES: Cn1c(C2CC2)nc(-c2ccc3c(c2)CN(C2CCC(=O)NC2=O)C3=O)c1-c1cccnc1]

A. 3-(2-Cyclopropyl-1-methyl-1H-imidazol-5-yl)pyridine: To a solution of 2-cyclopropyl-1-methyl-imidazole (600 mg, 4.91 mmol, 1.00 eq), PCy₃ (138 mg, 491 μmol, 159 μL, 0.10 eq) and NaOtBu (1.42 g, 14.7 mmol, 3.00 eq) in o-xylene (30.0 mL) was added 3-bromopyridine (2.33 g, 14.7 mmol, 1.42 mL, 3.00 eq) and Pd(OAc)₂ (221 mg, 982 μmol, 0.20 eq) under N₂. The mixture was stirred at 130° C. under N₂ for 12 h. Then the mixture was filtered to collect liquid and concentrated under reduced pressure to get residue. The residue was purified by preparative-HPLC (using a Welch Ultimate XB-SiOH (250×50×10 μm) and gradient of 15-45% EtOH+MeOH in water over 25 min at a flow rate of 25 mL/min) to give the title compound (360 mg, 1.79 mmol, 39.6% yield, 99.1% purity in LCMS at 220 nm) as yellow oil. ¹H NMR (400 MHz, DMSO-d₆): δ 8.66 (s, 1H), 8.55-8.53 (m, 1H), 7.89-7.86 (m, 1H), 7.48-7.45 (m, 1H), 6.95 (s, 1H), 3.66 (s, 3H), 2.06-1.99 (m, 1H), 0.948-0.942 (m, 2H), 0.87-0.85 (m, 2H). (ESI⁺) m/z: 200.0 (M+H)⁺, (C₁₂H₁₃N₃).

B. 3-(4-Bromo-2-cyclopropyl-1-methyl-1H-imidazol-5-yl)pyridine: To a solution of 3-(2-cyclopropyl-3-methyl-imidazol-4-yl)pyridine (360 mg, 1.81 mmol, 1.00 eq) in ACN (4.50 mL) was added NBS (354 mg, 1.99 mmol, 1.10 eq) at 0° C. Then the mixture was stirred at 20° C. for 2 h. The reaction mixture was poured into H₂O (30.0 mL) and extracted with DCM (3×20.0 mL). The combined organic layer was washed with saturated NaCl aqueous (3×15.0 mL), dried over Na₂SO₄ and concentrated under reduced pressure to get residue. Then the residue was purified by preparative-TLC (Petroleum ether/Ethyl acetate=0/1, R_{f}=0.25) to get title compound (450 mg, 1.61 mmol, 89.0% yield, 99.4% purity in LCMS at 220 nm) as yellow oil. ¹H NMR (400 MHz, DMSO-d₆): δ 8.64-8.61 (m, 2H), 7.90-7.87 (m, 1H), 7.55-7.52 (m, 1H), 3.58 (s, 3H), 2.10-2.04 (m, 1H), 0.98-0.95 (m, 2H), 0.89-0.88 (m, 2H). (ESI⁺) m/z: 278.0 (M+H)⁺, (C₁₂H₁₂BrN₃).

C. 3-(5-(2-Cyclopropyl-1-methyl-5-(pyridin-3-yl)-1H-imidazol-4-yl)-1-oxoisoindolin-2-yl)piperidine-2,6-dione: To a solution of 3-(4-bromo-2-cyclopropyl-1-methyl-1H-imidazol-5-yl)pyridine (300 mg, 1.08 mmol, 1.00 eq), 3-(1-oxo-5-(4,4,5,5-tetramethyl-1,3,2-dioxaborolan-2-yl)isoindolin-2-yl)piperidine-2,6-dione (799 mg, 2.16 mmol, 2.00 eq) and K₃PO₄ (458 mg, 2.16 mmol, 2.00 eq) in dioxane (6.00 mL) and H₂O (0.30 mL) was added Ru-Phos-Pd-G₃, K₃PO₄ (180 mg, 216 μmol, 0.20 eq) under N₂. The mixture was stirred at 100° C. for 2.5 h under N₂. Then the mixture was filtered to collect filtered liquid, and the liquid was concentrated under reduced pressure to get residue. The residue was purified by preparative-HPLC (using a Phenomenex luna C18 (150×25 mm×5 μm) and gradient of 0-21% acetonitrile in water containing 0.05% FA over 15 min at a flow rate of 25 mL/min to give the title compound (113 mg, 255 μmol, 23.6% yield, 100% purity in HPLC at 220 nm) as white solid. ¹H NMR (400 MHz, DMSO-d₆): δ 11.0 (s, 1H), 8.68-8.67 (m, 1H), 8.57-8.56 (m, 1H), 7.87-7.85 (m, 1H), 7.56-7.53 (m, 3H), 7.35-7.33 (m, 1H), 5.09-5.04 (m, 1H), 4.37-4.32 (m, 1H), 4.25-4.18 (m, 1H), 3.49 (s, 3H), 2.90-2.89 (m, 1H), 2.86-2.56 (m, 1H), 2.36-2.32 (m, 1H), 2.11-2.08 (m, 1H), 1.97-1.96 (m, 1H), 1.02-0.98 (m, 4H). (ESI⁺) m/z: 442.0 (M+H)⁺, (C₂₅H₂₃N₅O₃).

### Example 45

### Synthesis of 3-(5-(2-Cyclopropyl-1-methyl-5-(pyridin-4-yl)-1H-imidazol-4-yl)-1-oxoisoindolin-2-yl)piperidine-2,6-dione

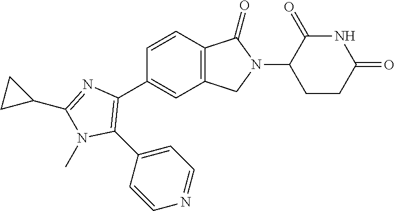 [SMILES: Cn1c(C2CC2)nc(-c2ccc3c(c2)CN(C2CCC(=O)NC2=O)C3=O)c1-c1ccncc1]

A. 4-(2-Cyclopropyl-1-methyl-1H-imidazol-5-yl)pyridine: To a solution of 2-cyclopropyl-1-methyl-imidazole (600 mg, 4.91 mmol, 1.00 eq), PCy₃ (138 mg, 491 μmol, 159 μL, 0.10 eq) and NaOtBu (1.42 g, 14.7 mmol, 3.00 eq) in o-xylene (30.0 mL) was added 4-bromopyridine (2.33 g, 14.7 mmol, 3.00 eq) and Pd(OAc)₂ (221 mg, 982 μmol, 0.20 eq) under N₂. The mixture was stirred at 130° C. for 12 h under N₂. Then the mixture was filtered to collect liquid and concentrated under reduced pressure to get residue. The residue was purified by column chromatography (SiO₂, DCM:MeOH=10:1, R_{f}=0.35) to give the title compound (856 mg, 3.83 mmol, 78.0% yield, 89.2% purity in LCMS at 220 nm) as yellow oil. ¹H NMR (400 MHz, DMSO-d₆): δ 8.58-8.56 (m, 2H), 7.47-7.46 (m, 2H), 7.11 (s, 1H), 3.74 (s, 3H), 2.07-2.02 (m, 1H), 0.95-0.93 (m, 2H), 0.86-0.84 (m, 2H). (ESI⁺) m/z: 200.0 (M+H)⁺, (C₁₂H₁₃N₃).

B. 4-(4-Bromo-2-cyclopropyl-1-methyl-1H-imidazol-5-yl)pyridine: To a solution of 4-(2-cyclopropyl-1-methyl-1H-imidazol-5-yl)pyridine (300 mg, 1.43 mmol, 1.00 eq) in MeOH (3.0 mL), then NBS (255 mg, 1.43 mmol, 1.00 eq) was added to the reaction mixture. The mixture was stirred at 25° C. for 8 h. The reaction mixture was poured into H₂O (30.0 mL) and extracted with DCM (3×20.0 mL). The combined organic layer was washed with saturated NaCl aqueous (3×15.0 mL), dried over Na₂SO₄ and concentrated under reduced pressure to get residue. Then the residue was purified by preparative-TLC (Petroleum ether/ethyl acetate=0/1, R_{f}=0.25) to get title compound (450 mg, 1.61 mmol, 72.8% yield, 99.4% purity in LCMS at 220 nm) as yellow oil. ¹H NMR (400 MHz, DMSO-d₆): δ 8.64-8.61 (m, 2H), 7.90-7.87 (m, 1H), 7.55-7.52 (m, 1H), 3.58 (s, 3H), 2.10-2.04 (m, 1H), 0.97-0.95 (m, 2H), 0.89-0.88 (m, 2H). (ESI⁺) m/z: 278.0 (M+H)⁺, (C₁₂H₁₂BrN₃).

C. 3-(5-(2-Cyclopropyl-1-methyl-5-(pyridin-4-yl)-1H-imidazol-4-yl)-1-oxoisoindolin-2-yl)piperidine-2,6-dione: To a solution of 4-(4-bromo-2-cyclopropyl-1-methyl-1H-imidazol-5-yl)pyridine (270 mg, 970 μmol, 1.00 eq), 3-(1-oxo-5-(4,4,5,5-tetramethyl-1,3,2-dioxaborolan-2-yl)isoindolin-2-yl)piperidine-2,6-dione (719 mg, 1.94 mmol, 2.00 eq) and K₃PO₄ (412 mg, 1.94 mmol, 2.00 eq) in dioxane (5.40 mL) and H₂O (0.27 mL) was added Ru-Phos-Pd-G₃ (162 mg, 194 μmol, 0.20 eq) under N₂. The mixture was stirred at 100° C. for 2 h under N₂. Then the mixture was filtered to collect filtered liquid, and the liquid was concentrated under reduced pressure to get residue. The residue was purified by preparative-HPLC (using a Phenomenex luna C18 (150×25 mm×5 μm) and gradient of 0-21% acetonitrile in water containing 0.05% FA over 15 min at a flow rate of 25 mL/min) to give the title compound (242 mg, 548 μmol, 56.5% yield, 100% purity in HPLC at 220 nm) as white solid. 1H NMR (400 MHz, DMSO-d₆): δ 11.0 (s, 1H), 8.68-8.64 (m, 2H), 7.57-7.44 (m, 2H), 7.41-7.35 (m, 3H), 5.09-5.04 (m, 1H), 4.38-4.17 (m, 2H), 3.54 (s, 3H), 2.90-2.60 (m, 1H), 2.37-2.36 (m, 1H), 2.12-2.10 (m, 1H), 2.33-2.32 (m, 1H), 2.08-1.97 (m, 1H), 1.06-0.98 (m, 4H). (ESI⁺) m/z: 442.0 (M+H)⁺, (C₂₅H₂₃N₅O₃).

### Example 46

### Synthesis of 3-(5-(2-Cyclopropyl-1-methyl-5-(1-methyl-1H-pyrazol-4-yl)-1H-imidazol-4-yl)-1-oxoisoindolin-2-yl)piperidine-2,6-dione

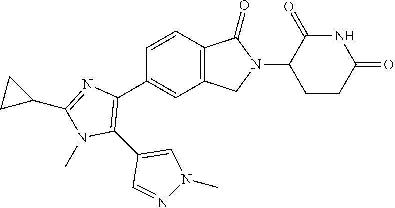 [SMILES: Cn1cc(-c2c(-c3ccc4c(c3)CN(C3CCC(=O)NC3=O)C4=O)nc(C3CC3)n2C)cn1]

A. 4-(2-Cyclopropyl-1-methyl-1H-imidazol-5-yl)-1-methyl-1H-pyrazole: To a solution of 2-cyclopropyl-1-methyl-imidazole (0.30 g, 2.46 mmol, 1.00 eq) and 4-bromo-1-methyl-pyrazole (1.19 g, 7.37 mmol, 3.00 eq) in o-xylene (15.0 mL) was added PCy₃ (68.8 mg, 245 μmol, 79.6 μL, 0.10 eq), Pd(OAc)₂ (27.5 mg, 122 μmol, 0.05 eq) and tBuONa (707 mg, 7.37 mmol, 3.00 eq) under N₂. The reaction mixture was stirred at 130° C. for 12 h under N₂. The reaction mixture was concentrated in vacuum to give residue. The residue was purified by column chromatography (SiO₂, Dichloromethane:Methanol=100:1 to 20:1; TLC, Dichloromethane:Methanol=20:1, R_{f}=0.60) to give the title compound (0.40 g, 1.98 mmol, 80.5% yield) as colorless oil. (ESI⁺) m/z: 203.1 (M+H)⁺, (C₁₁H₁₄N₄).

B. 4-(4-Bromo-2-cyclopropyl-1-methyl-1H-imidazol-5-yl)-1-methyl-1H-pyrazole: To a solution of 2-cyclopropyl-1-methyl-5-(1-methylpyrazol-4-yl)imidazole (0.30 g, 1.48 mmol, 1.00 eq) in ACN (2.00 mL) was added a solution of NBS (158 mg, 889 μmol, 0.60 eq) in ACN (1.00 mL) at 0° C. The reaction mixture was stirred at 25° C. for 2 h. The reaction mixture was concentrated in vacuum to give residue. The residue was purified by preparative-TLC (SiO₂, Dichloromethane:Methanol=20:1, R_{f}=0.40) to give the title compound (120 mg, 426 μmol, 28.7% yield) as yellow oil. (ESI⁺) m/z: 281.0 (M+H)⁺, (C₁₁H₁₃BrN₄).

C. 3-(5-(2-Cyclopropyl-1-methyl-5-(1-methyl-1H-pyrazol-4-yl)-1H-imidazol-4-yl)-1-oxoisoindolin-2-yl)piperidine-2,6-dione: To a solution of 4-bromo-2-cyclopropyl-1-methyl-5-(1-methylpyrazol-4-yl)imidazole (120 mg, 426 μmol, 1.00 eq) and 3-[1-oxo-5-(4,4,5,5-tetramethyl-1,3,2-dioxaborolan-2-yl)isoindolin-2-yl]piperidine-2,6-dione (189 mg, 512 μmol, 1.20 eq) in dioxane (1.00 mL) and H₂O (0.05 mL) was added K₃PO₄ (181 mg, 853 μmol, 2.00 eq) and Ru-Phos-Pd-G₃ (35.7 mg, 42.6 μmol, 0.10 eq) under N₂. The reaction mixture was stirred at 100° C. for 2 h under N₂. The reaction mixture was concentrated in vacuum to give residue. The residue was purified by preparative-HPLC (using a Phenomenex Luna (200 mm×40 mm×10 m) and gradient of 1-25% acetonitrile in water containing 0.05% TFA over 10 min at a flow rate of 25 mL/min to give the title compound (40.3 mg, 86.4 μmol, 20.2% yield, 95.3% purity in HPLC at 220 nm) as off-white solid. ¹H NMR: (400 MHz, DMSO-d₆) δ 11.0 (s, 1H), 8.03 (s, 1H), 7.76 (d, J=7.6 Hz, 1H), 7.69 (s, 1H), 7.61 (s, 1H), 7.55 (d, J=8.0 Hz, 1H), 5.14-5.09 (m, 1H), 4.47-4.30 (m, 2H), 3.90 (s, 3H), 3.66 (s, 3H), 2.96-2.88 (m, 1H), 2.62-2.50 (m, 1H), 2.41-2.32 (m, 2H), 2.05-2.02 (m, 1H), 1.32-1.22 (m, 4H). (ESI⁺) m/z: 445.0 (M+H)⁺, (C₂₄H₂₄N₆O₃).

### Example 47

### Synthesis of 3-(5-(3-Cyclopropyl-1H-pyrazol-4-yl)-1-oxoisoindolin-2-yl)piperidine-2,6-dione

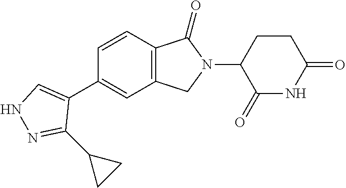 [SMILES: O=C1CCC(N2Cc3cc(-c4c[nH]nc4C4CC4)ccc3C2=O)C(=O)N1]

A. 3-(5-(3-Cyclopropyl-1H-pyrazol-4-yl)-1-oxoisoindolin-2-yl)piperidine-2,6-dione: To a solution of 4-bromo-3-cyclopropyl-1H-pyrazole (100 mg, 534 μmol, 1.00 eq) in dioxane (5.00 mL) and H₂O (0.20 mL) was added 3-[1-oxo-5-(4,4,5,5-tetramethyl-1,3,2-dioxaborolan-2-yl)isoindolin-2-yl]piperidine-2,6-dione (395 mg, 1.07 mmol, 2.00 eq), Ru-Phos-Pd-G₃ (151 mg, 213 μmol, 151 μL, 0.40 eq) and K₃PO₄ (340 mg, 1.60 mmol, 3.00 eq) under N₂. The mixture was stirred at 100° C. for 2 h under N₂. After the reaction was completed, the reaction mixture was filtered and the filtrate was concentrated under vacuum to give a residue. The residue was purified by preparative-TLC (SiO₂, Petroleum ether: ethyl acetate=0:1, R_{f}=0:1, R_{f}=0.40) and preparative-HPLC (using a Phenomenex Luna C18 (150 mm×25 mm×10 μm) and gradient of 10-40% acetonitrile in water containing 0.1% TFA over 15 min at a flow rate of 25 mL/min) to give the title compound (12.6 mg, 35.3 μmol, 3.36% yield, 98.3% purity in HPLC at 220 nm) as a white solid. ¹H NMR: (400 MHz, DMSO-d₆) δ 10.9 (s, 1H), 7.90 (d, J=2.4 Hz, 1H), 7.81 (s, 1H), 7.37 (t, J=8.4 Hz, 2H), 5.12 (dd, J=9.2, 4.8 Hz, 1H), 4.41 (dd, J=53.2, 16.8 Hz, 2H), 2.98-2.92 (m, 1H), 2.62 (s, 1H), 2.44-2.39 (m, 1H), 2.06-2.00 (m, 2H), 0.96 (d, J=6.8 Hz, 2H), 0.82 (s, 2H). (ESI⁺) m/z: 351.1 (M+H)⁺, (C₁₉H₁₈N₄O₃).

### Example 48

### Synthesis of 3-(5-(1-Cyclohexyl-1H-imidazol-2-yl)-1-oxoisoindolin-2-yl)piperidine-2,6-dione

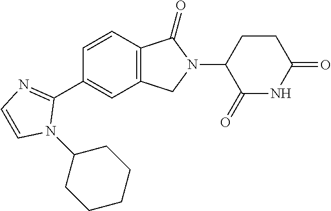 [SMILES: O=C1CCC(N2Cc3cc(-c4nccn4C4CCCCC4)ccc3C2=O)C(=O)N1]

A. tert-Butyl 5-amino-4-(5-(1-cyclohexyl-1H-imidazol-2-yl)-1-oxoisoindolin-2-yl)-5-oxopentanoate: To a solution of tert-butyl 5-amino-4-(5-bromo-1-oxoisoindolin-2-yl)-5-oxopentanoate (400 mg, 1.01 mmol, 1.00 eq) and 1-cyclohexyl-1H-imidazole (212 mg, 1.41 mmol, 1.40 eq) in dioxane (16.0 mL) was added CuI (384 mg, 2.01 mmol, 2.00 eq), PPh₃ (26.4 mg, 101 μmol, 0.10 eq) and DBU (307 mg, 2.01 mmol, 304 μL, 2.00 eq). Then the mixture was added Pd(OAc)₂ (56.5 mg, 252 μmol, 0.25 eq) under N₂. Then the mixture was stirred at 140° C. for 18 h. The reaction mixture was concentrated under reduced pressure to get residue. The residue was purified by column chromatography (SiO₂, Dichloromethane:Methanol=100/1 to 15/1, TLC:Dichloromethane:Methanol=10/1, R_{f}=0.40) to give the title compound (220 mg, 380 μmol, 37.7% yield, 80.6% purity in LCMS at 220 nm) as yellow solid. (ESI⁺) m/z: 467.1 (M+H)⁺, (C₂₆H₃₄N₄O₄).

B. 3-(5-(1-Cyclohexyl-1H-imidazol-2-yl)-1-oxoisoindolin-2-yl)piperidine-2,6-dione): To a solution of tert-butyl 5-amino-4-(5-(1-cyclohexyl-1H-imidazol-2-yl)-1-oxoisoindolin-2-yl)-5-oxopentanoate (220 mg, 380 μmol, 1.00 eq) and TsOH (131 mg, 760 μmol, 2.00 eq) in ACN (5.00 mL). Then the mixture was stirred at 80° C. for 4 h. The reaction mixture was concentrated under reduced pressure to get residue. The residue was purified by preparative-HPLC (using a Phenomenex luna C18 (150×25 mm×5 μm) and gradient of 10.0%-40.0% acetonitrile in water containing 0.50% TFA over 15 min at a flow rate of 25 mL/min) to give the title compound (47.6 mg, 121 μmol, 22.7% yield, 100% purity in HPLC at 220 nm) as white solid. ¹H NMR: (400 MHz, DMSO-d₆) δ 11.0 (s, 1H), 8.14 (s, 1H), 8.12-8.00 (m, 2H), 7.91-7.83 (m, 2H), 5.21-5.16 (m, 1H), 4.63-4.46 (m, 2H), 4.18-4.12 (m, 1H), 2.94-2.92 (m, 1H), 2.69-2.65 (m, 1H), 2.43-2.40 (m, 1H), 2.04-1.99 (m, 3H), 1.84-1.77 (m, 4H), 1.68-1.66 (m, 1H), 1.33-1.19 (m, 3H). (ESI⁺) m/z: 393.2 (M+H)⁺, (C₂₂H₂₄N₄O₃).

### Example 49

### Synthesis of 3-(5-(2-Cyclohexyl-1-methyl-5-phenyl-1H-imidazol-4-yl)-1-oxoisoindolin-2-yl)piperidine-2,6-dione

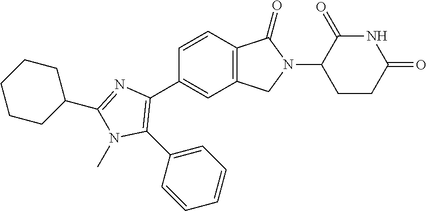 [SMILES: Cn1c(C2CCCCC2)nc(-c2ccc3c(c2)CN(C2CCC(=O)NC2=O)C3=O)c1-c1ccccc1]

A. N-(2,2-Dimethoxyethyl)-N-methylcyclohexanecarboximidamide: 2,2-dimethoxy-N-methyl-ethanamine (3.00 g, 25.1 mmol, 3.24 mL, 1.00 eq) was charged into a round-bottomed flask followed by cyclohexanecarbonitrile (3.44 g, 31.4 mmol, 3.74 mL, 1.25 eq) and CuCl (3.12 g, 31.4 mmol, 1.25 eq). The reaction mixture was stirred at 85° C. for 12 h to give the title compound (3.00 g, crude) as brown oil. (ESI⁺) m/z: 229.1 (M+H)⁺, (C₁₂H₂₄N₂O₂).

B. 2-Cyclohexyl-1-methyl-1H-imidazole: To a solution of N-(2,2-dimethoxyethyl)-N-methyl-cyclohexanecarboxamidine (3.00 g, 13.1 mmol, 1.00 eq) in MeOH (15.0 mL) was added conc HCl (3.00 mL, 2.74 eq). The reaction mixture was concentrated in vacuum to give residue. To the reaction mixture was added 50% aq NaOH (5.00 g) at 0° C., then added TMBE (30.0 mL) and stirred at 20° C. for 5 min. The reaction mixture was filtered to give a solid, which was washed with TMBE (2×15.0 mL) and dried in vacuum. The residue was purified by column chromatography (SiO₂, Dichloromethane:Methanol=100:1 to 20:1, R_{f}=0.20) to give the title compound ((120 mg, 730 μmol, 5.56% yield) as yellow oil. ¹H NMR: (400 MHz, CDCl₃) δ 6.92 (s, 1H), 6.74 (s, 1H), 3.58 (s, 3H), 2.65-2.60 (m, 1H), 1.89-1.85 (m, 3H), 1.74-1.70 (m, 1H), 1.66-1.63 (m, 2H), 1.37-1.32 (m, 4H). (ESI⁺) m/z: 165.1 (M+H)⁺, (C₁₀H₁₆N₂).

C. 2-Cyclohexyl-1-methyl-5-phenyl-1H-imidazole: To a solution of 2-cyclohexyl-1-methyl-imidazole (120 mg, 730 μmol, 1.00 eq) and bromobenzene (344 mg, 2.19 mmol, 230 μL, 3.00 eq) in DMF (1.00 mL) was added Pd(OAc)₂ (16.4 mg, 73.0 μmol, 0.10 eq), P(oxole)₃ (33.9 mg, 146 μmol, 0.20 eq) and K₂CO₃ (201 mg, 1.46 mmol, 2.00 eq) under N₂. The reaction mixture was stirred at 100° C. for 12 h under N₂. The reaction mixture was concentrated in vacuum to give residue. The residue was purified by preparative-TLC (SiO₂, Dichloromethane:Methanol=20:1, R_{f}=0.25) to give the title compound (55.0 mg, 228 μmol, 31.3% yield) as yellow solid. ¹H NMR: (400 MHz, DMSO-d₆) δ 7.47-7.42 (m, 4H), 7.38-7.34 (m, 1H), 6.89 (s, 1H), 3.55 (s, 3H), 2.80-2.74 (m, 1H), 1.88-1.81 (m, 2H), 1.80-1.78 (m, 2H), 1.65-1.60 (m, 1H), 1.55-1.51 (m, 2H), 1.50-1.41 (m, 2H), 1.38-1.26 (m, 1H). (ESI⁺) m/z: 201.1 (M+H)⁺, (C₁₆H₂₀N₂).

D. 4-bromo-2-cyclohexyl-1-methyl-5-phenyl-1H-imidazole: A solution of NBS (42.7 mg, 240 μmol, 1.05 eq) in ACN (1.00 mL) was added to the reaction mixture of 2-cyclohexyl-1-methyl-5-phenyl-imidazole (55.0 mg, 228 μmol, 1.00 eq) in ACN (1.00 mL) at 0° C. The reaction mixture was stirred at 25° C. for 2 h. The reaction mixture was poured into H₂O (5.00 mL), extracted with EtOAc (3×5.00 mL), dried over Na₂SO₄ and concentrated in vacuum to give a residue. The residue was purified by preparative-TLC (SiO₂, Dichloromethane:Methanol=20:1; TLC, Dichloromethane:Methanol=20:1, R_{f}=0.50) to give the title compound (52.0 mg, 162 μmol, 71.1% yield) as colorless oil. ¹H NMR: (400 MHz, DMSO-d₆) δ 7.51-7.47 (m, 2H), 7.44-7.41 (m, 3H), 3.46 (s, 3H), 2.82-2.75 (m, 1H), 1.87-1.67 (m, 5H), 1.51-1.47 (m, 2H), 1.38-1.36 (m, 2H), 1.35-1.24 (m, 1H). (ESI⁺) m/z: 319.0 (M+H)⁺, (C₁₆H₁₉BrN₂).

E. 3-(5-(2-cyclohexyl-1-methyl-5-phenyl-1H-imidazol-4-yl)-1-oxoisoindolin-2-yl)piperidine-2,6-dione: To a solution of 4-bromo-2-cyclohexyl-1-methyl-5-phenyl-imidazole (50.0 mg, 156 μmol, 1.00 eq), 3-[1-oxo-5-(4,4,5,5-tetramethyl-1,3,2-dioxaborolan-2-yl)isoindolin-2-yl]piperidine-2,6-dione (86.9 mg, 234 μmol, 1.50 eq) in dioxane (1.00 mL) and H₂O (0.05 mL) was added Ru-Phos-Pd-G₃ (13.1 mg, 15.6 μmol, 0.10 eq), K₃PO₄ (66.4 mg, 313 μmol, 2.00 eq) under N₂. The reaction mixture was stirred at 100° C. for 2 h under N₂. The reaction mixture was concentrated in vacuum to give a residue. The residue was purified by preparative-HPLC (using a Phenomenex luna C18 (150 mm×25 mm×5 μm) and gradient of 10-40% acetonitrile in water (TFA) over 10 min at a flow rate of 25 mL/min to give the title compound (30.3 mg, 62.7 μmol, 40.0% yield, 100% purity in HPLC at 220 nm) as white solid. ¹H NMR: (400 MHz, DMSO-d₆) δ 10.9 (s, 1H), 7.68 (s, 1H), 7.58-7.55 (m, 4H), 7.50-7.45 (m, 2H), 7.44-7.41 (m, 1H), 5.11-5.07 (m, 1H), 4.41-4.24 (m, 2H), 3.57 (s, 3H), 2.90-2.60 (m, 1H), 2.59-2.50 (m, 1H), 2.43-2.38 (m, 2H), 2.03-2.00 (m, 3H), 1.98-1.87 (m, 2H), 1.87-1.72 (m, 3H), 1.72-1.47 (m, 2H), 1.45-1.30 (m, 1H). (ESI⁺) m/z: 483.2 (M+H)⁺, (C₂₉H₃₀N₄O₃).

### Example 50

### Synthesis of 3-(5-(1-methyl-5-phenyl-2-(tetrahydro-2H-pyran-4-yl)-1H-imidazol-4-yl)-1-oxoisoindolin-2-yl)piperidine-2,6-dione

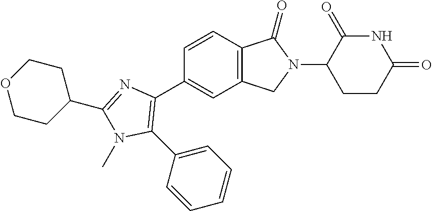 [SMILES: Cn1c(C2CCOCC2)nc(-c2ccc3c(c2)CN(C2CCC(=O)NC2=O)C3=O)c1-c1ccccc1]

A. N-(2,2-Dimethoxyethyl)-N-methylcyclohexanecarboximidamide: 2,2-dimethoxy-N-methylethan-1-amine (3.00 g, 25.1 mmol, 3.24 mL, 1.00 eq) was charged into a round-bottomed flask followed by tetrahydro-2H-pyran-4-carbonitrile (3.50 g, 31.4 mmol, 1.25 eq) and CuCl (3.12 g, 31.4 mmol, 1.25 eq). The reaction mixture was stirred at 85° C. for 12 h to give the title compound (3.00 g, crude) as brown oil. (ESI⁺) m/z: 229.1 (M+H)⁺, (C₁₁H₂₂N₂O₃).

B. 1-Methyl-2-(tetrahydro-2H-pyran-4-yl)-1H-imidazole: To a solution of N-(2,2-dimethoxyethyl)-N-methylcyclohexanecarboximidamide (3.00 g, 13.1 mmol, 1.00 eq) in MeOH (15.0 mL) was added conc HCl (3.00 mL, 2.74 eq). The reaction mixture was concentrated in vacuum to give residue. To the reaction mixture was added 50% aq NaOH (5.00 g) at 0° C., then added TMBE (30.0 mL) and stirred at 20° C. for 5 min. The reaction mixture was filtered to give a solid, which was washed with TMBE (2×15.0 mL) and dried in vacuum. The residue was purified by column chromatography (SiO₂, Dichloromethane:Methanol=100:1 to 20:1; TLC, Dichloromethane:Methanol=20:1, R_{f}=0.25) to give the title compound (400 mg, 2.41 mmol, 18.4% yield) as yellow oil. ¹H NMR: (400 MHz, CDCl₃) δ 6.95 (s, 1H), 6.78 (s, 1H), 4.10-4.07 (m, 2H), 3.62 (s, 3H), 3.56-2.50 (m, 2H), 2.90-2.86 (m, 1H), 2.09-2.02 (m, 2H), 1.80-1.77 (m, 2H). (ESI⁺) m/z: 167.1 (M+H)⁺, (C₉H₁₄N₂O).

C. 1-Methyl-5-phenyl-2-(tetrahydro-2H-pyran-4-yl)-1H-imidazole: To a solution of 1-methyl-2-(tetrahydro-2H-pyran-4-yl)-1H-imidazole (400 mg, 2.41 mmol, 1.00 eq) and bromobenzene (1.13 g, 7.22 mmol, 3.00 eq) in DMF (4.00 mL) was added Pd(OAc)₂ (54.3 mg, 240 μmol, 0.10 eq), P(oxole)₃ (111 mg, 481 μmol, 0.20 eq) and K₂CO₃ (665 mg, 4.81 mmol, 2.00 eq) under N₂. The reaction mixture was stirred at 100° C. for 12 h under N₂. The reaction mixture was concentrated in vacuum to give residue. The residue was purified by column chromatography (SiO₂, Dichloromethane:Methanol=100:1 to 80:1; TLC, Dichloromethane:Methanol=20:1, R_{f}=0.40) to give the title compound (200 mg, 825 μmol, 34.3% yield) as yellow solid. ¹H NMR: (400 MHz, DMSO-d₆) δ 7.48-7.42 (m, 4H), 6.91 (s, 1H), 3.96-3.92 (m, 2H), 3.58 (s, 3H), 3.51-3.45 (m, 2H), 3.07-3.04 (m, 1H), 1.81-1.76 (m, 4H). (ESI⁺) m/z: 243.6 (M+H)⁺, (C₁₅H₁₁N₂O).

D. 4-Bromo-1-methyl-5-phenyl-2-(tetrahydro-2H-pyran-4-yl)-1H-imidazole: A solution of NBS (115 mg, 649 μmol, 1.05 eq) in ACN (1.00 mL) was added to the reaction mixture of 1-methyl-5-phenyl-2-(tetrahydro-2H-pyran-4-yl)-1H-imidazole (150 mg, 619 μmol, 1.00 eq) in ACN (1.00 mL) at 0° C. The reaction mixture was stirred at 25° C. for 2 h. The reaction mixture was poured into H₂O (5.00 mL), extracted with EtOAc (3×5.00 mL), dried over Na₂SO₄ and concentrated in vacuum to give a residue. The residue was purified by preparative-TLC (SiO₂, Dichloromethane:Methanol=20:1; TLC, Dichloromethane:Methanol=20:1, R_{f}=0.50) to give the title compound (150 mg, 466 μmol, 75.4% yield) as colorless oil. ¹H NMR: (400 MHz, DMSO-d₆) δ 7.50-7.48 (m, 2H), 7.45-7.41 (m, 3H), 3.95-3.90 (m, 2H), 3.48 (s, 3H), 3.46-3.42 (m, 2H), 3.33-3.07 (m, 1H), 1.78-1.69 (m, 4H). (ESI⁺) m/z: 321.0 (M+H)⁺, (C₁₅H₁₇BrN₂O).

E. 3-(5-(1-Methyl-5-phenyl-2-(tetrahydro-2H-pyran-4-yl)-1H-imidazol-4-yl)-1-oxoisoindolin-2-yl)piperidine-2,6-dione: To a solution of 4-bromo-1-methyl-5-phenyl-2-(tetrahydro-2H-pyran-4-yl)-1H-imidazole (150 mg, 466 μmol, 1.00 eq), 3-[1-oxo-5-(4,4,5,5-tetramethyl-1,3,2-dioxaborolan-2-yl)isoindolin-2-yl]piperidine-2,6-dione (259 mg, 700 μmol, 1.50 eq) in dioxane (2.00 mL) and H₂O (0.10 mL) was added Ru-Phos-Pd-G₃ (39.1 mg, 46.6 μmol, 0.10 eq), K₃PO₄ (198 mg, 933 μmol, 2.00 eq) under N₂. The reaction mixture was stirred at 100° C. for 2 h under N₂. The reaction mixture was concentrated in vacuum to give a residue. The residue was purified by preparative-HPLC (using a Phenomenex luna C18 (150 mm×25 mm×5 μm) and gradient of 10-40% acetonitrile in water (TFA) over 10 min at a flow rate of 25 mL/min to give the title compound (88.2 mg, 182 μmol, 39.0% yield, 100% purity in HPLC at 220 nm) as white solid. ¹H NMR: (400 MHz, MeOD) δ 7.79 (d, J=8.0 Hz, 1H), 7.59-7.56 (m, 4H), 7.54-7.52 (m, 1H), 7.46-7.44 (m, 2H), 5.17-5.12 (m, 1H), 4.45-4.43 (m, 2H), 4.14-4.11 (m, 2H), 3.72 (s, 3H), 3.69-3.63 (m, 3H), 2.86-2.78 (m, 2H), 2.14-2.10 (m, 1H), 2.08-2.00 (m, 5H). (ESI⁺) m/z: 485.2 (M+H)⁺, (C₂₈H₂₈N₄O₄).

### Example 51

### Synthesis of 3-(5-(2-Ethyl-5-phenyl-2H-1,2,3-triazol-4-yl)-1-oxoisoindolin-2-yl)piperidine-2,6-dione

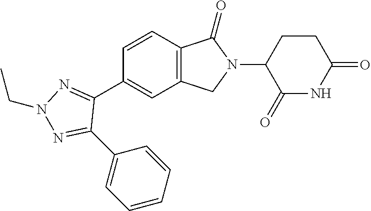 [SMILES: CCn1nc(-c2ccccc2)c(-c2ccc3c(c2)CN(C2CCC(=O)NC2=O)C3=O)n1]

A. 4-Iodo-5-phenyl-2H-1,2,3-triazole: To a solution of 4-phenyl-2H-1,2,3-triazole (600 mg, 4.13 mmol, 1.00 eq) in MeCN (20.0 mL) was added NIS (1.21 g, 5.37 mmol, 1.30 eq) at 0° C. The mixture was stirred at 50° C. for 72 h. The reaction mixture was poured into H₂O (25.0 mL) and extracted with Ethyl acetate (3×30.0 mL). The combined organic layer was washed with brine (3×20.0 mL), dried over Na₂SO₄, filtered and concentrated. The residue was purified by column chromatography (SiO₂, Dichloromethane:Methanol=15:1, TLC:Dichloromethane:Methanol=15:1, R_{f}=0.40) to give the title compound (500 mg, 1.82 mmol, 44.0% yield, 98.7% purity in LCMS at 220 nm) as light yellow solid. (ESI⁺) m/z: 271.7 (M+H)⁺, (C₈H₆IN₃).

B. 2-Ethyl-4-iodo-5-phenyl-2H-1,2,3-triazole: To a solution of 4-iodo-5-phenyl-2H-1,2,3-triazole (440 mg, 1.62 mmol, 1.00 eq), K₂CO₃ (112 mg, 811 μmol, 0.50 eq) and bromoethane (194 mg, 1.79 mmol, 133 μL, 1.10 eq) in DMF (10.0 mL). The mixture was stirred at 25° C. for 30 h. The reaction mixture was poured into H₂O (20.0 mL) and extracted with Ethyl acetate (3×20.0 mL). The combined organic layer was washed with brine (3×20.0 mL), dried over Na₂SO₄, filtered and concentrated. The crude product was purified by preparative-TLC (Petroleum ether:Ethyl acetate=3:1, R_{f}=0.70) to give the title compound (280 mg, 928 μmol, 57.2% yield, 99.2% purity in LCMS at 220 nm) as yellow solid. (ESI⁺) m/z: 299.8 (M+H)⁺, (C₁₀H₁₀IN₃).

C. 3-(5-(2-Ethyl-5-phenyl-2H-1,2,3-triazol-4-yl)-1-oxoisoindolin-2-yl)piperidine-2,6-dione: To a solution of 2-ethyl-4-iodo-5-phenyl-2H-1,2,3-triazole (200 mg, 660 μmol, 1.00 eq), 3-(1-oxo-5-(4,4,5,5-tetramethyl-1,3,2-dioxaborolan-2-yl)isoindolin-2-yl)piperidine-2,6-dione (495 mg, 1.34 mmol, 2.00 eq) and K₃PO₄ (425 mg, 2.01 mmol, 3.00 eq) in dioxane (5.00 mL) and H₂O (0.25 mL) was added Ru-Phos-Pd-G₃ (55.9 mg, 66.8 μmol, 0.10 eq) under N₂. The mixture was stirred at 100° C. for 2 h under N₂. The reaction mixture was concentrated under reduced pressure to get a residue at 45° C. The residue was purified by preparative-HPLC (using a Phenomenex luna C18 (150×25 mm×10 μm) and gradient of 30.0%-60.0% acetonitrile in water containing 0.50% TFA over 15 min at a flow rate of 25 mL/min) to give the title compound (106 mg, 255 μmol, 38.1% yield, 100% purity in HPLC at 220 nm) as white solid. ¹H NMR: (400 MHz, DMSO-d₆) δ 11.0-10.9 (m, 1H), 7.76-7.73 (m, 2H), 7.61-7.56 (m, 1H), 7.45-7.37 (m, 5H), 5.14-5.10 (m, 1H), 4.61-4.51 (m, 2H), 4.49-4.29 (m, 2H), 2.91-2.80 (m, 1H), 2.63-2.58 (m, 1H), 2.41-2.37 (m, 1H), 2.09-1.97 (m, 1H), 1.60-1.53 (m, 3H). (ESI⁺) m/z: 416.0 (M+H)⁺, (C₂₃H₂IN₅O₃).

### Example 52

### Synthesis of 3-(5-(1-Methyl-5-phenyl-2-(trifluoromethyl)-1H-imidazol-4-yl)-1-oxoisoindolin-2-yl)piperidine-2,6-dione

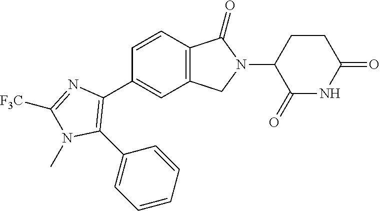 [SMILES: Cn1c(C(F)(F)F)nc(-c2ccc3c(c2)CN(C2CCC(=O)NC2=O)C3=O)c1-c1ccccc1]

A. 1-Methyl-5-phenyl-2-(trifluoromethyl)-1H-imidazole: To a solution of 4-phenyl-2H-1, 2, 3-triazole (50 mg, 344 μmol, 1.00 eq) in DCE (1.50 mL) was added TFAA (180 mg, 861 μmol, 119 μL, 2.50 eq). Then the mixture was stirred at 50° C. for 15 h. Then MeNH₂ (69.7 mg, 1.03 mmol, 3.00 eq) was added into the mixture. Then the mixture was stirred at 140° C. in MW for 3 h. The reaction mixture was concentrated under reduced pressure to get a residue at 45° C. The residue was purified by preparative-TLC (Petroleum ether:Ethyl acetate=5:1, R_{f}=0.50) to give the title compound (86.0 mg, 356 μmol, 25.8% yield, 93.8% purity in LCMS at 220 nm) as yellow solid. (ESI⁺) m/z: 227.0 (M+H)⁺, (C₁₁H₉F₃N₂).

B. 4-Bromo-1-methyl-5-phenyl-2-(trifluoromethyl)-1H-imidazole: To a solution of 1-methyl-5-phenyl-2-(trifluoromethyl)-1H-imidazole (110 mg, 486 μmol, 1.00 eq) in ACN (3.00 mL) was added NBS (95.2 mg, 534 μmol, 1.10 eq) at 0° C. The mixture was stirred at 25° C. for 3 h. The reaction mixture was concentrated under reduced pressure to get a residue at 45° C. The residue was purified by preparative-TLC (Petroleum ether:Ethyl acetate=5:1, R_{f}=0.60) to give the title compound (86.0 mg, 356 μmol, 70.1% yield, 93.8% purity in LCMS at 220 nm) as light yellow solid. ¹H NMR: (400 MHz, CDCl₃) δ 7.56-7.51 (m, 3H), 7.49-7.39 (m, 2H), 3.71-3.66 (m, 3H). (ESI⁺) m/z: 303.9 (M+H)⁺, (C₁₁H₈BrF₃N₂).

C. 3-(5-(1-Methyl-5-phenyl-2-(trifluoromethyl)-1H-imidazol-4-yl)-1-oxoisoindolin-2-yl)piperidine-2,6-dione: To a solution of 4-bromo-1-methyl-5-phenyl-2-(trifluoromethyl)-1H-imidazole (90.0 mg, 294 μmol, 1.00 eq), 3-(1-oxo-5-(4,4,5,5-tetramethyl-1,3,2-dioxaborolan-2-yl)isoindolin-2-yl)piperidine-2,6-dione (218 mg, 589 μmol, 2.00 eq) and K₃PO₄ (187 mg, 884 μmol, 3.00 eq) in dioxane (5.00 mL) and H₂O (0.25 mL) was added Ru-Phos-Pd-G₃ (24.6 mg, 29.5 μmol, 0.10 eq) under N₂. The mixture was stirred at 100° C. for 2 h under N₂. The reaction mixture was concentrated under reduced pressure to get a residue at 45° C. The residue was purified by preparative-HPLC (using a Phenomenex luna C18 (150×25 mm×5 μm) and gradient of 34.0%-64.0% acetonitrile in water containing 0.50% TFA over 10 min at a flow rate of 25 mL/min) to give the title compound (59.3 mg, 126 μmol, 42.7% yield, 98.4% purity in HPLC at 220 nm) as white solid. ¹H NMR: (400 MHz, DMSO-d₆) δ 11.0-10.9 (m, 1H), 7.63-7.50 (m, 7H), 7.48-7.40 (m, 1H), 5.11-5.05 (m, 1H), 4.43-4.21 (m, 2H), 3.56 (s 3H), 2.95-2.84 (m, 1H), 2.60-2.53 (m, 1H), 2.38-2.33 (m, 1H), 1.99-1.91 (m, 1H). (ESI⁺) m/z: 468.1 (M+H)⁺, (C₂₄H₁₉F₃N₄O₃).

### Example 53

### Synthesis of 3-(1-Oxo-5-(5-phenyl-2-(trifluoromethyl)-1H-imidazol-4-yl)isoindolin-2-yl)piperidine-2,6-dione

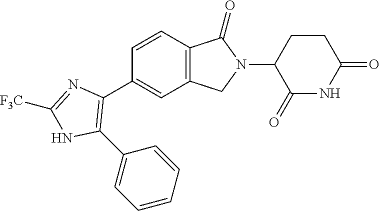 [SMILES: O=C1CCC(N2Cc3cc(-c4nc(C(F)(F)F)[nH]c4-c4ccccc4)ccc3C2=O)C(=O)N1]

A. 5-Phenyl-2-(trifluoromethyl)-1H-imidazole: To a solution of 4-phenyl-2H-1,2,3-triazole (250 mg, 1.72 mmol, 1.00 eq) in DCE (7.50 mL) was added TFAA (904 mg, 4.31 mmol, 598 μL, 2.50 eq). Then the mixture was stirred at 50° C. for 15 h. Then AcONH₄ (663 mg, 8.61 mmol, 5.00 eq) was added into the mixture. Then the mixture was stirred at 140° C. in MW for 2 h. The reaction mixture was concentrated under reduced pressure to get a residue at 45° C. The residue was purified by preparative-TLC (Petroleum ether:Ethyl acetate=5:1, R_{f}=0.50) to give the title compound (287 mg, 1.34 mmol, 38.8% yield, 99.0% purity in LCMS at 220 nm) as light yellow solid. (ESI⁺) m/z: 212.9 (M+H)⁺, (C₁₀H₇F₃N₂).

B. 4-Bromo-5-phenyl-2-(trifluoromethyl)-1H-imidazole: To a solution of 5-phenyl-2-(trifluoromethyl)-1H-imidazole (280 mg, 1.31 mmol, 1.00 eq) in ACN (5.00 mL) was added NBS (323 mg, 1.31 mmol, 1.00 eq) at 0° C. The mixture was stirred at 0° C. for 0.5 h. The reaction mixture was concentrated under reduced pressure to get a residue at 45° C. The residue was purified by preparative-TLC (Petroleum ether:Ethyl acetate=5:1,R_{f}=0.50) to give the title compound (314 mg, 1.07 mmol, 82.4% yield, 99.6% purity in LCMS at 220 nm) as light yellow solid. ¹H NMR: (400 MHz, CDCl₃) δ 9.98-9.89 (In, 1H), 7.68-7.64 (m, 2H), 7.52-7.43 (m, 3H). (ESI⁺) m/z: 292.7 (M+H)⁺, (C₁₀H₆BrF₃N₂).

C. 3-(1-Oxo-5-(5-phenyl-2-(trifluoromethyl)-1H-imidazol-4-yl)isoindolin-2-yl)piperidine-2,6-dione: To a solution of 4-bromo-5-phenyl-2-(trifluoromethyl)-1H-imidazole (150 mg, 513 μmol, 1.00 eq), 3-(1-oxo-5-(4,4,5,5-tetramethyl-1,3,2-dioxaborolan-2-yl)isoindolin-2-yl)piperidine-2,6-dione (380 mg, 1.03 mmol, 2.00 eq) and K₃PO₄ (326 mg, 1.54 mmol, 3.00 eq) in dioxane (5.00 mL) and H₂O (0.25 mL) was added Ru-Phos-Pd-G₃ (214 mg, 256 μmol, 0.50 eq) under N₂. The mixture was stirred at 100° C. for 2 h under N₂. The reaction mixture was concentrated under reduced pressure to get a residue at 45° C. The residue was purified by preparative-HPLC (using a Phenomenex luna C18 (150×25 mm×5 μm) and gradient of 24.0%-54.0% acetonitrile in water containing 0.50% TFA over 10 min at a flow rate of 25 mL/min) to give the title compound (111 mg, 244 μmol, 47.5% yield, 100% purity in HPLC at 220 nm) as white solid. ¹H NMR: (400 MHz, DMSO-d₆) δ 11.1-10.9 (m, 1H), 7.80-7.67 (m, 2H), 7.56-7.33 (m, 6H), 5.11-5.03 (m, 1H), 4.49-4.28 (m, 2H), 2.94-2.87 (m, 1H), 2.61-2.57 (m, 1H), 2.38-2.32 (m, 1H), 2.11-1.91 (m, 1H). (ESI⁺) m/z: 455.2 (M+H)⁺, (C₂₃H₁₇F₃N₄O₃).

### Example 54

### Synthesis of 3-(1-Oxo-5-(2-phenyl-1H-imidazol-4-yl)isoindolin-2-yl)piperidine-2,6-dione

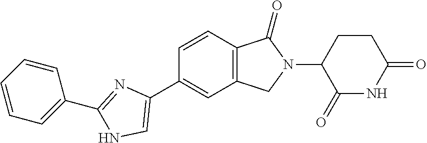 [SMILES: O=C1CCC(N2Cc3cc(-c4c[nH]c(-c5ccccc5)n4)ccc3C2=O)C(=O)N1]

A. 3-(1-Oxo-5-(2-phenyl-1H-imidazol-4-yl)isoindolin-2-yl)piperidine-2,6-dione: To a solution of 4-bromo-2-phenyl-1H-imidazole (150 mg, 672 μmol, 1.00 eq), 3-[1-oxo-5-(4,4,5,5-tetramethyl-1,3,2-dioxaborolan-2-yl)isoindolin-2-yl]piperidine-2,6-dione (497 mg, 1.34 mmol, 2.00 eq) in dioxane (2.00 mL) and H₂O (0.10 mL) was added Ru-Phos-Pd-G₃ (112 mg, 134 μmol, 0.20 eq), K₃PO₄ (285 mg, 1.34 mmol, 2.00 eq) under N₂. The reaction mixture was stirred at 100° C. for 2 h under N₂. The reaction mixture was concentrated in vacuum to give residue. The residue was purified by preparative-HPLC (using a Welch Xtimate C18 (150 mm×25 mm×10 μm) and gradient of 6-36% acetonitrile in water containing 0.05% TFA over 10 min at a flow rate of 25 mL/min to give the title compound (46.3 mg, 118 μmol, 17.5% yield, 98.5% purity in HPLC at 220 nm) as white solid. ¹H NMR: (400 MHz, DMSO-d₆) δ 11.0 (s, 1H), 8.17 (s, 1H), 8.12 (s, 1H), 8.06-8.03 (m, 3H), 7.83-7.81 (m, 1H), 7.60-7.54 (m, 3H), 5.16-5.12 (m, 1H), 4.56-4.38 (m, 2H), 2.96-2.90 (m, 1H), 2.64-2.59 (m, 1H), 2.46-2.43 (m, 1H), 2.04-2.02 (m, 1H). (ESI⁺) m/z: 386.9 (M+H)⁺, (C₂₂H₁₈N₄O₃).

### Examples 55-116

The compounds of Examples 55-116 were prepared according to the following

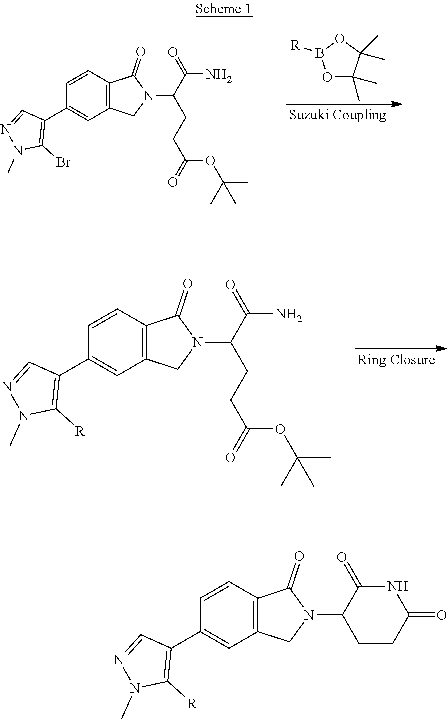

Step 1 Suzuki Coupling

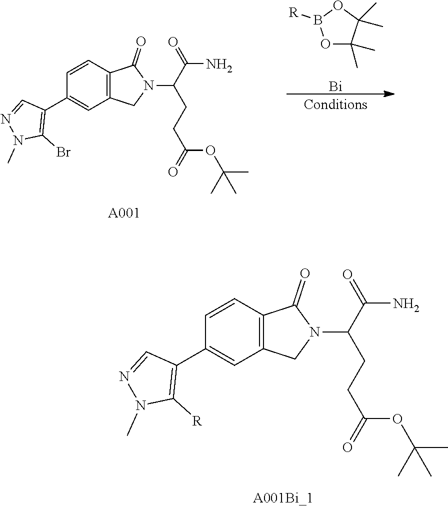

Condition 1: To a vial containing a solution of A001 (150 μmol, 1.00 eq) and Bi (180 μmol, 1.20 eq) in Dioxane (1.20 mL) was added K₃PO₄ (1.5 M in H₂O, 450 μmol, 3.00 eq), and Pd-118 (15.0 μmol, 0.10 eq) under protection of N₂. The mixture was stirred at 65° C. for 16 hrs. The reaction mixture was diluted with H₂O (2.00 mL) and extracted with ethyl acetate (2.00 mL×3). The combined organic layers were washed with brine (2.00 mL), dried over Na₂SO₄, filtered and concentrated under reduced pressure to give crude intermediates used for next step.

Condition 2: To a vial containing a solution of A001 (150 μmol, 1.00 eq) and Bi (180 μmol, 1.20 eq) in Dioxane (1.20 mL) was added K₃PO₄ (1.5 M in H₂O, 450 μmol, 3.00 eq), and Pd-118 (15.0 μmol, 0.10 eq) under protection of N₂. The mixture was stirred at 120° C. for 2 hours under microwave. The reaction mixture was diluted with H₂O (2.00 mL) and extracted with ethyl acetate (2.00 mL×3). The combined organic layers were washed with brine (2.00 mL), dried over Na₂SO₄, filtered and concentrated under reduced pressure to give crude intermediates used for next step.

Step 2 Ring Closure:

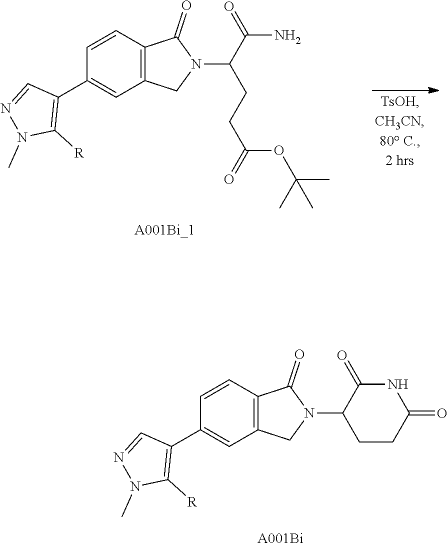

Conditions: To a vial containing a solution of A001Bi_1 (˜150 μmol, 1.00 eq) in CH₃CN (1.50 mL) was added TsOH (1.50 mmol, 10.0 eq). The mixture was stirred at 80° C. for 2 hrs. The residue was concentrated under reduced pressure and purified by prep-HPLC to give final product.

The following compounds were synthesized according to the above method:

| | | MW + 1 | Step 1 |
|---|---|---|---|
| Example | Structure | (Observed) | Conditions |
| 55 | 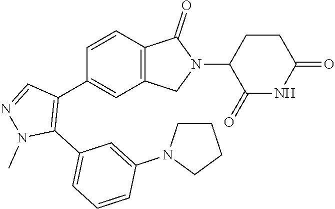 [SMILES: Cn1ncc(-c2ccc3c(c2)CN(C2CCC(=O)NC2=O)C3=O)c1-c1cccc(N2CCCC2)c1] | 470.2 | 1 |
| 56 | 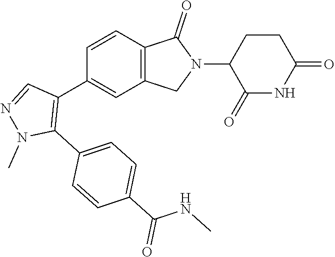 [SMILES: CNC(=O)c1ccc(-c2c(-c3ccc4c(c3)CN(C3CCC(=O)NC3=O)C4=O)cnn2C)cc1] | 458.3 | 1 |
| 57 | 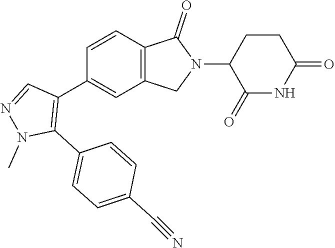 [SMILES: Cn1ncc(-c2ccc3c(c2)CN(C2CCC(=O)NC2=O)C3=O)c1-c1ccc(C#N)cc1] | 426.2 | 1 |
| 58 | 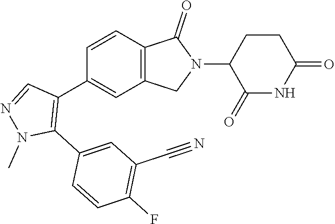 [SMILES: Cn1ncc(-c2ccc3c(c2)CN(C2CCC(=O)NC2=O)C3=O)c1-c1ccc(F)c(C#N)c1] | 444.2 | 1 |
| 59 | 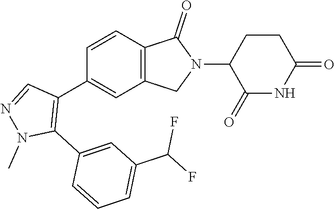 [SMILES: Cn1ncc(-c2ccc3c(c2)CN(C2CCC(=O)NC2=O)C3=O)c1-c1cccc(C(F)F)c1] | 451.2 | 1 |
| 60 | 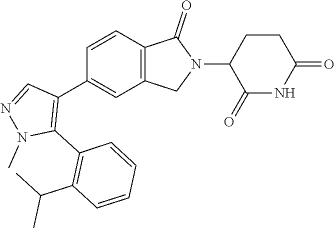 [SMILES: CC(C)c1ccccc1-c1c(-c2ccc3c(c2)CN(C2CCC(=O)NC2=O)C3=O)cnn1C] | 443.3 | 1 |
| 61 | 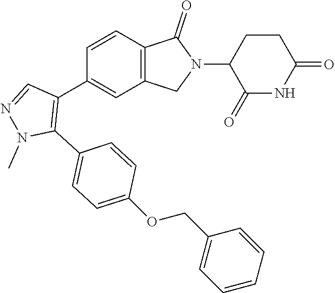 [SMILES: Cn1ncc(-c2ccc3c(c2)CN(C2CCC(=O)NC2=O)C3=O)c1-c1ccc(OCc2ccccc2)cc1] | 507.3 | 1 |
| 62 | 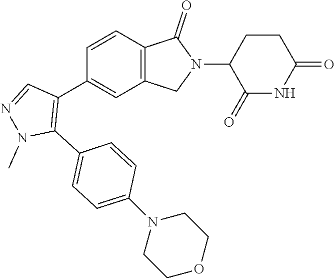 [SMILES: Cn1ncc(-c2ccc3c(c2)CN(C2CCC(=O)NC2=O)C3=O)c1-c1ccc(N2CCOCC2)cc1] | 486.3 | 1 |
| 63 | 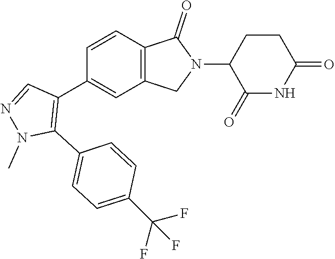 [SMILES: Cn1ncc(-c2ccc3c(c2)CN(C2CCC(=O)NC2=O)C3=O)c1-c1ccc(C(F)(F)F)cc1] | 469.2 | 1 |
| 64 | 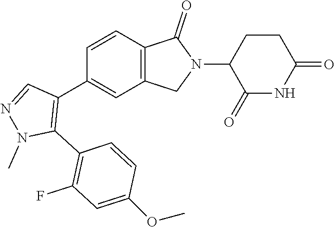 [SMILES: COc1ccc(-c2c(-c3ccc4c(c3)CN(C3CCC(=O)NC3=O)C4=O)cnn2C)c(F)c1] | 449.2 | 1 |
| 65 | 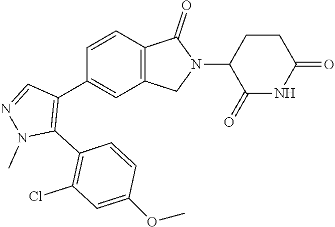 [SMILES: COc1ccc(-c2c(-c3ccc4c(c3)CN(C3CCC(=O)NC3=O)C4=O)cnn2C)c(Cl)c1] | 465.2 | 1 |
| 66 | 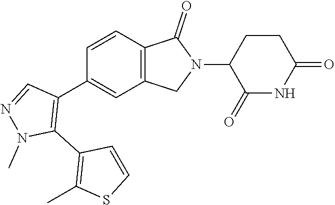 [SMILES: Cc1sccc1-c1c(-c2ccc3c(c2)CN(C2CCC(=O)NC2=O)C3=O)cnn1C] | 421.2 | 1 |
| 67 | 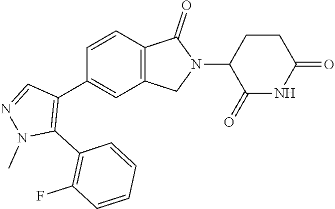 [SMILES: Cn1ncc(-c2ccc3c(c2)CN(C2CCC(=O)NC2=O)C3=O)c1-c1ccccc1F] | 419.2 | 1 |
| 68 | 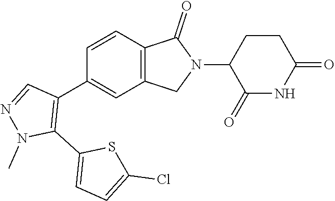 [SMILES: Cn1ncc(-c2ccc3c(c2)CN(C2CCC(=O)NC2=O)C3=O)c1-c1ccc(Cl)s1] | 441.1 | 1 |
| 69 | 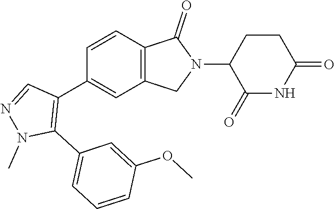 [SMILES: COc1cccc(-c2c(-c3ccc4c(c3)CN(C3CCC(=O)NC3=O)C4=O)cnn2C)c1] | 431.2 | 1 |
| 70 | 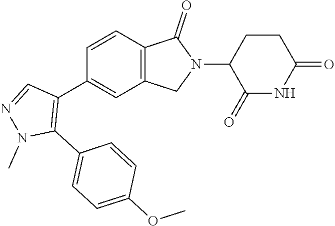 [SMILES: COc1ccc(-c2c(-c3ccc4c(c3)CN(C3CCC(=O)NC3=O)C4=O)cnn2C)cc1] | 431.2 | 1 |
| 71 | 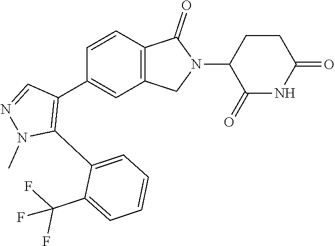 [SMILES: Cn1ncc(-c2ccc3c(c2)CN(C2CCC(=O)NC2=O)C3=O)c1-c1ccccc1C(F)(F)F] | 469.3 | 1 |
| 72 | 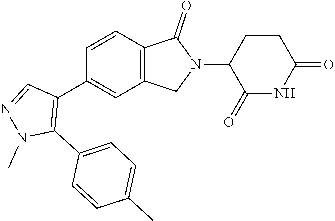 [SMILES: Cc1ccc(-c2c(-c3ccc4c(c3)CN(C3CCC(=O)NC3=O)C4=O)cnn2C)cc1] | 415.2 | 1 |
| 73 | 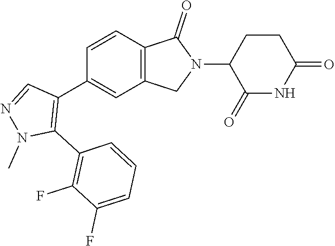 [SMILES: Cn1ncc(-c2ccc3c(c2)CN(C2CCC(=O)NC2=O)C3=O)c1-c1cccc(F)c1F] | 437.2 | 1 |
| 74 | 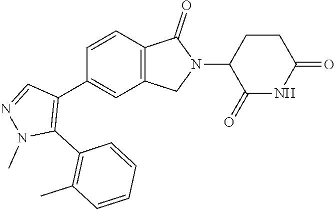 [SMILES: Cc1ccccc1-c1c(-c2ccc3c(c2)CN(C2CCC(=O)NC2=O)C3=O)cnn1C] | 415.3 | 1 |
| 75 | 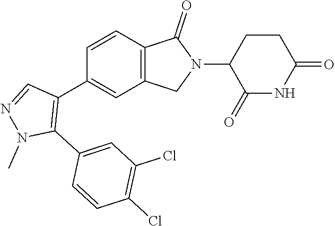 [SMILES: Cn1ncc(-c2ccc3c(c2)CN(C2CCC(=O)NC2=O)C3=O)c1-c1ccc(Cl)c(Cl)c1] | 469.1 | 1 |
| 76 | 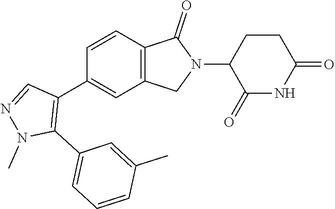 [SMILES: Cc1cccc(-c2c(-c3ccc4c(c3)CN(C3CCC(=O)NC3=O)C4=O)cnn2C)c1] | 415.3 | 1 |
| 77 | 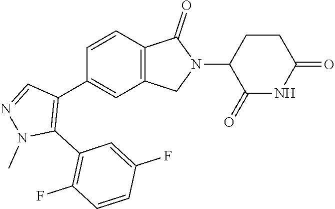 [SMILES: Cn1ncc(-c2ccc3c(c2)CN(C2CCC(=O)NC2=O)C3=O)c1-c1cc(F)ccc1F] | 437.3 | 1 |
| 78 | 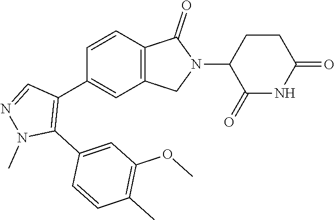 [SMILES: COc1cc(-c2c(-c3ccc4c(c3)CN(C3CCC(=O)NC3=O)C4=O)cnn2C)ccc1C] | 445.3 | 1 |
| 79 | 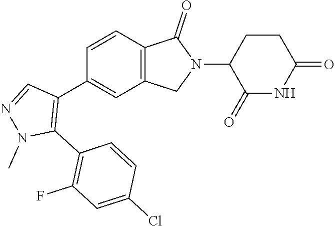 [SMILES: Cn1ncc(-c2ccc3c(c2)CN(C2CCC(=O)NC2=O)C3=O)c1-c1ccc(Cl)cc1F] | 453.2 | 1 |
| 80 | 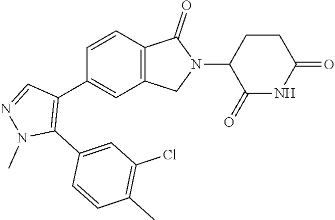 [SMILES: Cc1ccc(-c2c(-c3ccc4c(c3)CN(C3CCC(=O)NC3=O)C4=O)cnn2C)cc1Cl] | 449.2 | 1 |
| 81 | 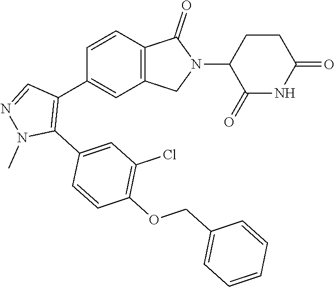 [SMILES: Cn1ncc(-c2ccc3c(c2)CN(C2CCC(=O)NC2=O)C3=O)c1-c1ccc(OCc2ccccc2)c(Cl)c1] | 541.3 | 1 |
| 82 | 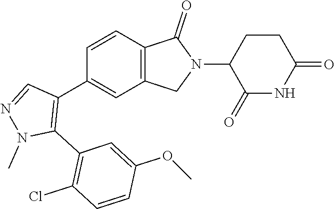 [SMILES: COc1ccc(Cl)c(-c2c(-c3ccc4c(c3)CN(C3CCC(=O)NC3=O)C4=O)cnn2C)c1] | 465.2 | 1 |
| 83 | 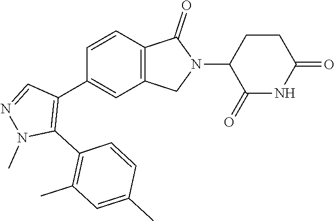 [SMILES: Cc1ccc(-c2c(-c3ccc4c(c3)CN(C3CCC(=O)NC3=O)C4=O)cnn2C)c(C)c1] | 429.3 | 1 |
| 84 | 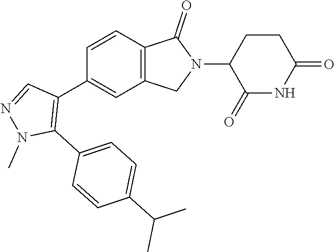 [SMILES: CC(C)c1ccc(-c2c(-c3ccc4c(c3)CN(C3CCC(=O)NC3=O)C4=O)cnn2C)cc1] | 443.3 | 1 |
| 85 | 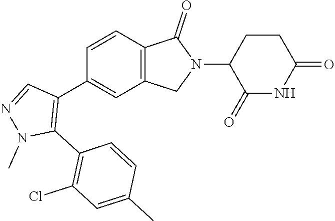 [SMILES: Cc1ccc(-c2c(-c3ccc4c(c3)CN(C3CCC(=O)NC3=O)C4=O)cnn2C)c(Cl)c1] | 449.2 | 1 |
| 86 | 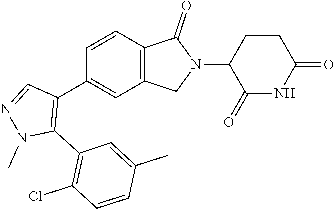 [SMILES: Cc1ccc(Cl)c(-c2c(-c3ccc4c(c3)CN(C3CCC(=O)NC3=O)C4=O)cnn2C)c1] | 449.2 | 1 |
| 87 | 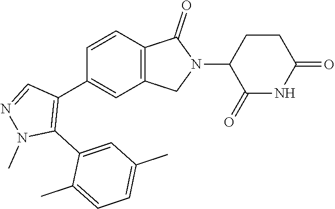 [SMILES: Cc1ccc(C)c(-c2c(-c3ccc4c(c3)CN(C3CCC(=O)NC3=O)C4=O)cnn2C)c1] | 429.3 | 1 |
| 88 | 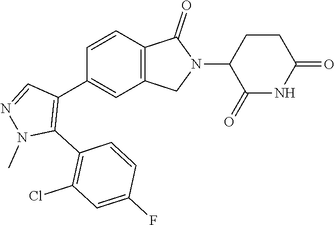 [SMILES: Cn1ncc(-c2ccc3c(c2)CN(C2CCC(=O)NC2=O)C3=O)c1-c1ccc(F)cc1Cl] | 453.2 | 1 |
| 89 | 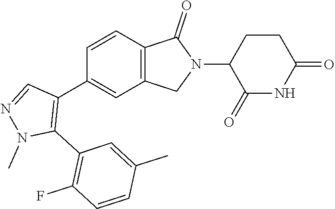 [SMILES: Cc1ccc(F)c(-c2c(-c3ccc4c(c3)CN(C3CCC(=O)NC3=O)C4=O)cnn2C)c1] | 433.2 | 1 |
| 90 | 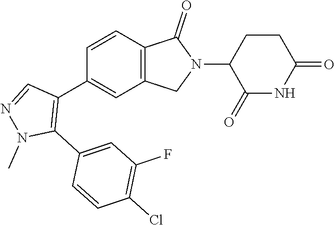 [SMILES: Cn1ncc(-c2ccc3c(c2)CN(C2CCC(=O)NC2=O)C3=O)c1-c1ccc(Cl)c(F)c1] | 453.2 | 1 |
| 91 | 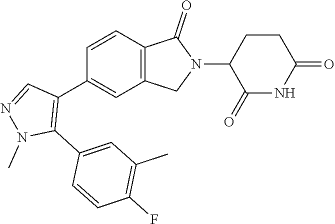 [SMILES: Cc1cc(-c2c(-c3ccc4c(c3)CN(C3CCC(=O)NC3=O)C4=O)cnn2C)ccc1F] | 433.2 | 1 |
| 92 | 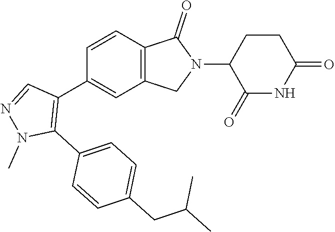 [SMILES: CC(C)Cc1ccc(-c2c(-c3ccc4c(c3)CN(C3CCC(=O)NC3=O)C4=O)cnn2C)cc1] | 457.3 | 1 |
| 93 | 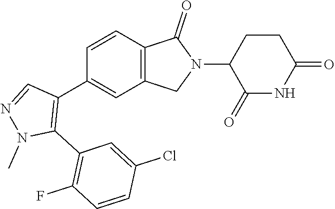 [SMILES: Cn1ncc(-c2ccc3c(c2)CN(C2CCC(=O)NC2=O)C3=O)c1-c1cc(Cl)ccc1F] | 453.2 | 1 |
| 94 | 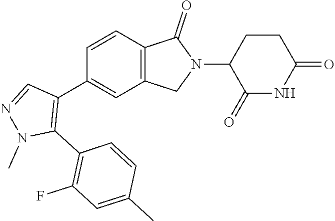 [SMILES: Cc1ccc(-c2c(-c3ccc4c(c3)CN(C3CCC(=O)NC3=O)C4=O)cnn2C)c(F)c1] | 433.2 | 1 |
| 95 | 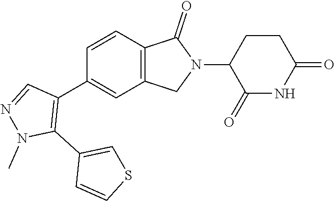 [SMILES: Cn1ncc(-c2ccc3c(c2)CN(C2CCC(=O)NC2=O)C3=O)c1-c1ccsc1] | 407.2 | 1 |
| 96 | 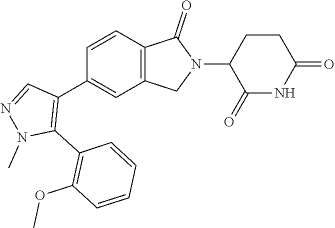 [SMILES: COc1ccccc1-c1c(-c2ccc3c(c2)CN(C2CCC(=O)NC2=O)C3=O)cnn1C] | 431.2 | 1 |
| 97 | 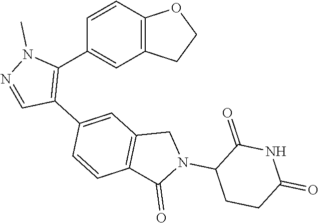 [SMILES: Cn1ncc(-c2ccc3c(c2)CN(C2CCC(=O)NC2=O)C3=O)c1-c1ccc2c(c1)CCO2] | 443.2 | 1 |
| 98 | 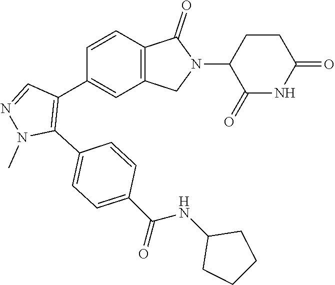 [SMILES: Cn1ncc(-c2ccc3c(c2)CN(C2CCC(=O)NC2=O)C3=O)c1-c1ccc(C(=O)NC2CCCC2)cc1] | 512.3 | 1 |
| 99 | 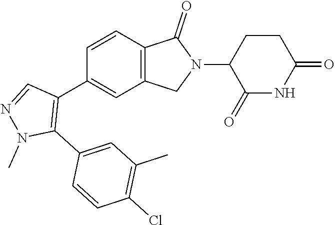 [SMILES: Cc1cc(-c2c(-c3ccc4c(c3)CN(C3CCC(=O)NC3=O)C4=O)cnn2C)ccc1Cl] | 449.2 | 1 |
| 100 | 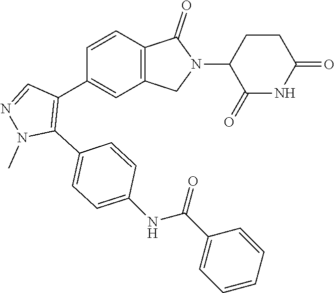 [SMILES: Cn1ncc(-c2ccc3c(c2)CN(C2CCC(=O)NC2=O)C3=O)c1-c1ccc(NC(=O)c2ccccc2)cc1] | 520.3 | 1 |
| 101 | 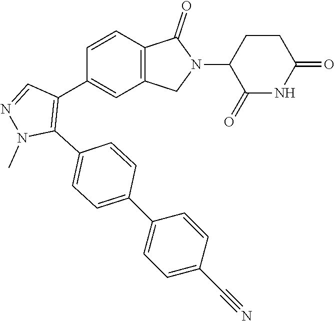 [SMILES: Cn1ncc(-c2ccc3c(c2)CN(C2CCC(=O)NC2=O)C3=O)c1-c1ccc(-c2ccc(C#N)cc2)cc1] | 502.3 | 1 |
| 102 | 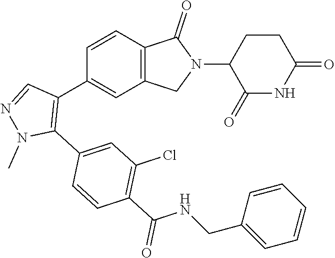 [SMILES: Cn1ncc(-c2ccc3c(c2)CN(C2CCC(=O)NC2=O)C3=O)c1-c1ccc(C(=O)NCc2ccccc2)c(Cl)c1] | 568.3 | 1 |
| 103 | 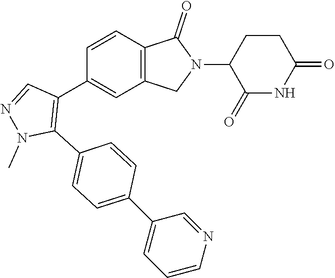 [SMILES: Cn1ncc(-c2ccc3c(c2)CN(C2CCC(=O)NC2=O)C3=O)c1-c1ccc(-c2cccnc2)cc1] | 478.3 | 1 |
| 104 | 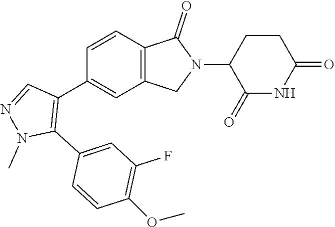 [SMILES: COc1ccc(-c2c(-c3ccc4c(c3)CN(C3CCC(=O)NC3=O)C4=O)cnn2C)cc1F] | 449.2 | 1 |
| 105 | 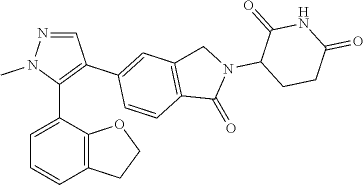 [SMILES: Cn1ncc(-c2ccc3c(c2)CN(C2CCC(=O)NC2=O)C3=O)c1-c1cccc2c1OCC2] | 443.3 | 1 |
| 106 | 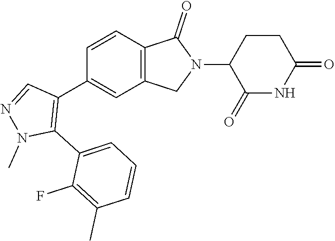 [SMILES: Cc1cccc(-c2c(-c3ccc4c(c3)CN(C3CCC(=O)NC3=O)C4=O)cnn2C)c1F] | 433.2 | 1 |
| 107 | 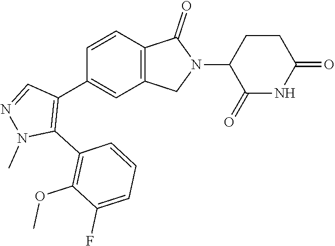 [SMILES: COc1c(F)cccc1-c1c(-c2ccc3c(c2)CN(C2CCC(=O)NC2=O)C3=O)cnn1C] | 449.2 | 1 |
| 108 | 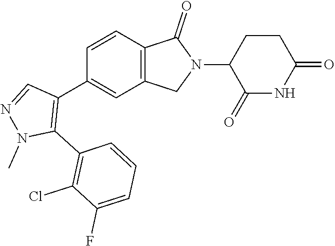 [SMILES: Cn1ncc(-c2ccc3c(c2)CN(C2CCC(=O)NC2=O)C3=O)c1-c1cccc(F)c1Cl] | 453.2 | 1 |
| 109 | 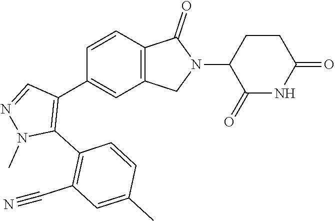 [SMILES: Cc1ccc(-c2c(-c3ccc4c(c3)CN(C3CCC(=O)NC3=O)C4=O)cnn2C)c(C#N)c1] | 440.3 | 1 |
| 110 | 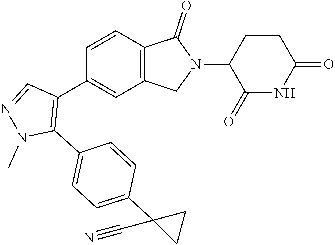 [SMILES: Cn1ncc(-c2ccc3c(c2)CN(C2CCC(=O)NC2=O)C3=O)c1-c1ccc(C2(C#N)CC2)cc1] | 466.3 | 1 |
| 111 | 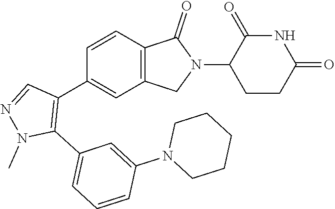 [SMILES: Cn1ncc(-c2ccc3c(c2)CN(C2CCC(=O)NC2=O)C3=O)c1-c1cccc(N2CCCCC2)c1] | 484.3 | 1 |
| 112 | 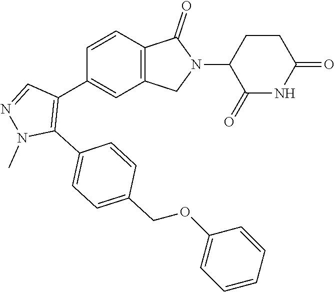 [SMILES: Cn1ncc(-c2ccc3c(c2)CN(C2CCC(=O)NC2=O)C3=O)c1-c1ccc(COc2ccccc2)cc1] | 507.3 | 1 |
| 113 | 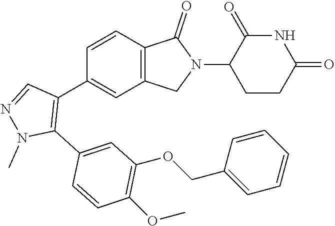 [SMILES: COc1ccc(-c2c(-c3ccc4c(c3)CN(C3CCC(=O)NC3=O)C4=O)cnn2C)cc1OCc1ccccc1] | 537.3 | 1 |
| 114 | 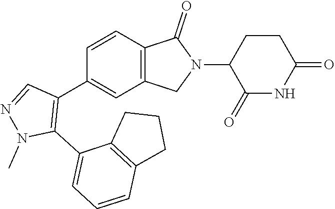 [SMILES: Cn1ncc(-c2ccc3c(c2)CN(C2CCC(=O)NC2=O)C3=O)c1-c1cccc2c1CCC2] | 441.3 | 1 |
| 115 | 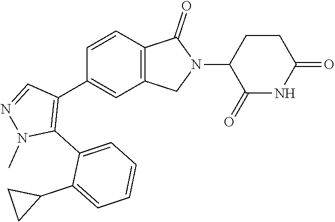 [SMILES: Cn1ncc(-c2ccc3c(c2)CN(C2CCC(=O)NC2=O)C3=O)c1-c1ccccc1C1CC1] | 441.3 | 1 |
| 116 | 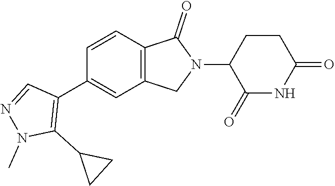 [SMILES: Cn1ncc(-c2ccc3c(c2)CN(C2CCC(=O)NC2=O)C3=O)c1C1CC1] | 365.2 | 1 |

### Example 117

### Synthesis of 3-(5-(2-cyclopropyl-1-methyl-5-(pyrimidin-5-yl)-1H-imidazol-4-yl)-1-oxoisoindolin-2-yl)piperidine-2,6-dione

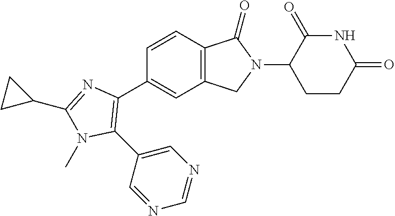 [SMILES: Cn1c(C2CC2)nc(-c2ccc3c(c2)CN(C2CCC(=O)NC2=O)C3=O)c1-c1cncnc1]

5-(2-Cyclopropyl-1-methyl-1H-imidazol-5-yl)pyrimidine: To a solution of 2-cyclopropyl-1-methyl-imidazole (0.30 g, 2.46 mmol, 1.00 eq) and 5-bromopyrimidine (1.17 g, 7.37 mmol, 3.00 eq) in DMF (2.00 mL) was added Pd(OAc)₂ (55.1 mg, 245 μmol, 0.10 eq), P(oxole)₃ (114 mg, 491 μmol, 0.20 eq) and K₂CO₃ (678 mg, 4.91 mmol, 2.00 eq) under N₂. The reaction mixture was stirred at 100° C. for 12 h under N₂. The reaction mixture was concentrated in vacuum to give residue. The residue was purified by preparative-TLC (SiO₂, Petroleum ether:Ethyl acetate=0:1, R_{f}=0.25) to give the title compound (0.40 g, 2.00 mmol, 81.3% yield) as yellow solid. ¹H NMR: (400 MHz, DMSO-d₆) δ 9.15 (s, 1H), 8.94 (s, 2H), 7.09 (s, 1H), 3.71 (s, 3H), 2.09-2.03 (m, 1H), 0.97-0.94 (m, 2H), 0.87-0.86 (m, 2H). (ESI⁺) m/z: 201.1 (M+H)⁺, (C₁₁H₁₂N₄).

B. 5-(4-Bromo-2-cyclopropyl-1-methyl-1H-imidazol-5-yl)pyrimidine: To a solution of 5-(2-cyclopropyl-3-methyl-imidazol-4-yl)pyrimidine (0.40 g, 2.00 mmol, 1.00 eq) in ACN (2.00 mL) was added a solution of NBS (426 mg, 2.40 mmol, 1.20 eq) in ACN (2.00 mL) at 0° C. The reaction mixture was stirred at 25° C. for 2 h. The reaction mixture was poured into water (10.0 mL), extracted with EtOAc (3×10.0 mL), dried over Na₂SO₄ and concentrated in vacuum to give a residue. The residue was purified by preparative-TLC (SiO₂, Methanol:Dichloromethane=20:1; TLC, Methanol:Dichloromethane=20:1, R_{f}=0.40) to give the title compound (245 mg, 877 μmol, 43.9% yield) as yellow solid. (ESI⁺) m/z: 279.0 (M+H)⁺, (C₁₁H₁₁BrN₄).

C. 3-(5-(2-Cyclopropyl-1-methyl-5-(pyrimidin-5-yl)-1H-imidazol-4-yl)-1-oxoisoindolin-2-yl)piperidine-2,6-dione: To a solution of 5-(5-bromo-2-cyclopropyl-3-methyl-imidazol-4-yl)pyrimidine (200 mg, 716 μmol, 1.00 eq), 3-[1-oxo-5-(4,4,5,5-tetramethyl-1,3,2-dioxaborolan-2-yl)isoindolin-2-yl]piperidine-2,6-dione (397 mg, 1.07 mmol, 1.50 eq) in dioxane (2.00 mL) and H₂O (0.10 mL) was added Ru-Phos-Pd-G₃ (119 mg, 143 μmol, 0.20 eq), K₃PO₄ (304 mg, 1.43 mmol, 2.00 eq) under N₂. The reaction mixture was stirred at 100° C. for 2 h under N₂. The reaction mixture was concentrated in vacuum to give residue. The residue was purified by preparative-HPLC (using a Phenomenex luna C18 (150 mm×25 mm×10 μm) and gradient of 4-34% acetonitrile in water (TFA) over 10 min at a flow rate of 25 mL/min to give the title compound (22.2 mg, 49.5 μmol, 6.92% yield, 98.6% purity in HPLC at 220 nm) as white solid. ¹H NMR: (400 MHz, DMSO-d₆) δ 10.9 (s, 1H), 9.32 (s, 1H), 8.89 (s, 2H), 7.65 (d, J=8.0 Hz, 1H), 7.57 (s, 1H), 7.40 (d, J=8.0 Hz, 1H), 5.10-5.06 (m, 1H), 4.41-4.23 (m, 2H), 3.63 (s, 3H), 2.93-2.86 (m, 1H), 2.60-2.50 (m, 1H), 2.38-2.32 (m, 2H), 2.05-1.98 (m, 1H), 1.40-1.00 (m, 4H). (ESI⁺) m/z: 443.2 (M+H)⁺, (C₂₄H₂₂N₆O₃).

### Example 118

### Synthesis of 3-(1-oxo-5-(3-(pyridin-2-yl)-1H-pyrazol-4-yl)isoindolin-2-yl)piperidine-2,6-dione

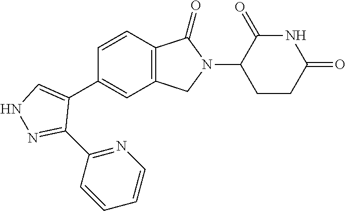 [SMILES: O=C1CCC(N2Cc3cc(-c4c[nH]nc4-c4ccccn4)ccc3C2=O)C(=O)N1]

A. 2-(4-Bromo-1-(tetrahydro-2H-pyran-2-yl)-1H-pyrazol-3-yl)pyridine: To a solution of 2-(4-bromo-1H-pyrazol-3-yl)pyridine (400 mg, 1.79 mmol, 1.00 eq), TFA (81.4 mg, 714 μmol, 53.1 μL, 0.40 eq) in Tol. (2.00 mL) and ACN (2.00 mL) was added DHP (1.40 g, 16.6 mmol, 1.52 mL, 9.30 eq) for several parts slowly at 25° C. The mixture was stirred at 100° C. for 8 h. The mixture was poured into H₂O (20.0 mL) and extracted with ethyl acetate (3×30.0 mL). The combined organic layer was washed with saturated NaCl aqueous (3×20.0 mL), dried over Na₂SO₄ and concentrated under reduced pressure to get residue. The residue was purified by column chromatography (SiO₂, Petroleum ether: ethyl acetated=15:1 to 8:1. Petroleum ether: ethyl acetated=1/1, R_{f}=0.35) to give the title compound (530 mg, 1.49 mmol, 83.2% yield, 86.4% purity in LCMS at 220 nm) was obtained as yellow oil. (ESI⁺) m/z: 309.9 (M+H)⁺, (C₁₃H₁₄BrN₃O).

B. 3-(1-Oxo-5-(3-(pyridin-2-yl)-1-(tetrahydro-2H-pyran-2-yl)-1H-pyrazol-4-yl) isoindolin-2-yl) piperidine-2, 6-dione: To a solution 2-(4-bromo-1-(tetrahydro-2H-pyran-2-yl)-1H-pyrazol-3-yl)pyridine (200 mg, 649 μmol, 1.00 eq), 3-(1-oxo-5-(4,4,5,5-tetramethyl-1,3,2-dioxaborolan-2-yl)isoindolin-2-yl)piperidine-2,6-dione (480 mg, 1.30 mmol, 2.00 eq) and K₃PO₄ (275 mg, 1.30 mmol, 2.00 eq) in dioxane (2.50 mL) and H₂O (0.13 mL) was added cataCXium A Pd G₃ (108.6 mg, 130 μmol, 0.20 eq) under N₂. The mixture was stirred at 100° C. for 2 h under N₂. The combined mixture was concentrated under reduced pressure to get residue. The residue was purified by preparative-TLC (Petroleum ether:Ethyl acetate=0:1, R_{f}=0.10) to give the title compound (25.0 mg, 41.9 μmol, 6.46% yield, 79.1% purity in LCMS at 220 nm) was obtained as brown solid. (ESI⁺) m/z: 472.2 (M+H)⁺, (C₂₆H₂₅N₅O₄).

C. 3-(1-Oxo-5-(3-(pyridin-2-yl)-1H-pyrazol-4-yl) isoindolin-2-yl) piperidine-2, 6-dione: To a solution of 3-(1-oxo-5-(3-(pyridin-2-yl)-1-(tetrahydro-2H-pyran-2-yl)-1H-pyrazol-4-yl) isoindolin-2-yl) piperidine-2, 6-dionee (21.0 mg, 44.5 μmol, 1.00 eq) in DCM (1.00 mL) was added HCl/dioxane (4.00 M, 100 μL, 9.00 eq) at 0° C. The mixture was stirred at 25° C. for 12 h. The mixture was concentrated under reduced pressure to get residue. The residue was purified by preparative-HPLC (using a Phenomenex luna C18 (150×25 mm×10 μm) and gradient of 0%-30.0% acetonitrile in water containing 0.50% TFA over 15 min at a flow rate of 25 mL/min) to give the title compound (6.00 mg, 14.9 μmol, 33.4% yield, 96.1% purity in HPLC at 220 nm) was obtained as white solid. ¹H NMR: (400 MHz, DMSO-d₆) δ 11.0 (s, 1H), 8.59-8.51 (m, 1H), 7.85-7.79 (m, 3H), 7.65-7.63 (m, 2H), 7.47-7.44 (m, 1H), 7.35 (s, 1H), 5.14-5.09 (m, 1H), 4.45-4.24 (m, 2H), 2.93-2.87 (m, 1H), 2.63-2.52 (m, 1H), 2.47-2.42 (m, 1H), 2.02-1.99 (m, 1H). (ESI⁺) m/z: 388.2 (M+H)⁺, (C₂₁H₁₇N₅O₃).

### Examples 119-160

The compounds of Examples 119-160 were prepared according to the method of Scheme 1 as shown for Examples 55-116.

| | | MW + 1 | Step 1 |
|---|---|---|---|
| Example | Structure | (Observed) | Conditions |
| 119 | 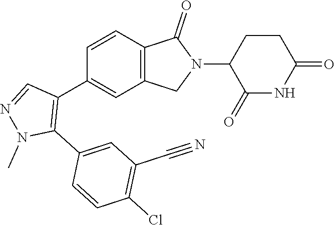 [SMILES: Cn1ncc(-c2ccc3c(c2)CN(C2CCC(=O)NC2=O)C3=O)c1-c1ccc(Cl)c(C#N)c1] | 460.2 | 1 |
| 120 | 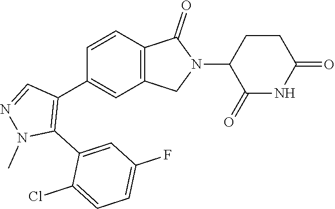 [SMILES: Cn1ncc(-c2ccc3c(c2)CN(C2CCC(=O)NC2=O)C3=O)c1-c1cc(F)ccc1Cl] | 453.2 | 1 |
| 121 | 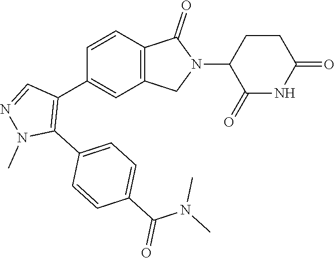 [SMILES: CN(C)C(=O)c1ccc(-c2c(-c3ccc4c(c3)CN(C3CCC(=O)NC3=O)C4=O)cnn2C)cc1] | 472.3 | 1 |
| 122 | 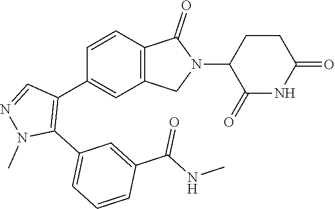 [SMILES: CNC(=O)c1cccc(-c2c(-c3ccc4c(c3)CN(C3CCC(=O)NC3=O)C4=O)cnn2C)c1] | 458.3 | 1 |
| 123 | 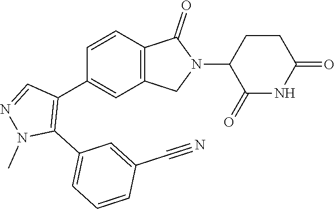 [SMILES: Cn1ncc(-c2ccc3c(c2)CN(C2CCC(=O)NC2=O)C3=O)c1-c1cccc(C#N)c1] | 426.2 | 1 |
| 124 | 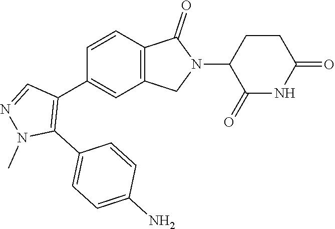 [SMILES: Cn1ncc(-c2ccc3c(c2)CN(C2CCC(=O)NC2=O)C3=O)c1-c1ccc(N)cc1] | 416.2 | 1 |
| 125 | 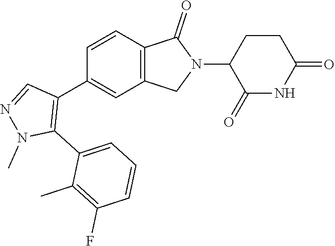 [SMILES: Cc1c(F)cccc1-c1c(-c2ccc3c(c2)CN(C2CCC(=O)NC2=O)C3=O)cnn1C] | 433.2 | 1 |
| 126 | 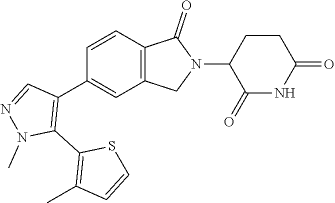 [SMILES: Cc1ccsc1-c1c(-c2ccc3c(c2)CN(C2CCC(=O)NC2=O)C3=O)cnn1C] | 421.2 | 1 |
| 127 | 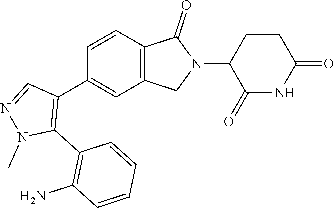 [SMILES: Cn1ncc(-c2ccc3c(c2)CN(C2CCC(=O)NC2=O)C3=O)c1-c1ccccc1N] | 416.2 | 1 |
| 128 | 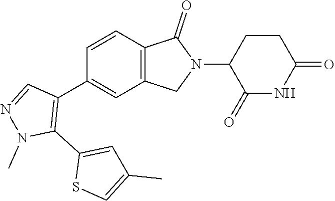 [SMILES: Cc1csc(-c2c(-c3ccc4c(c3)CN(C3CCC(=O)NC3=O)C4=O)cnn2C)c1] | 421.2 | 1 |
| 129 | 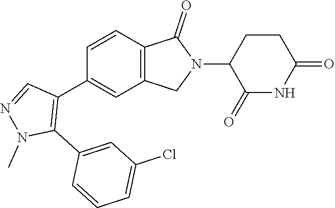 [SMILES: Cn1ncc(-c2ccc3c(c2)CN(C2CCC(=O)NC2=O)C3=O)c1-c1cccc(Cl)c1] | 435.2 | 1 |
| 130 | 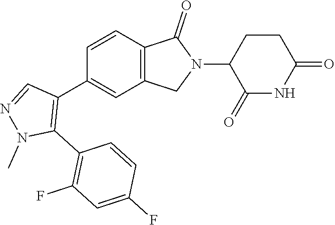 [SMILES: Cn1ncc(-c2ccc3c(c2)CN(C2CCC(=O)NC2=O)C3=O)c1-c1ccc(F)cc1F] | 437.2 | 1 |
| 131 | 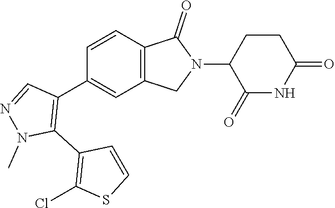 [SMILES: Cn1ncc(-c2ccc3c(c2)CN(C2CCC(=O)NC2=O)C3=O)c1-c1ccsc1Cl] | 441.1 | 1 |
| 132 | 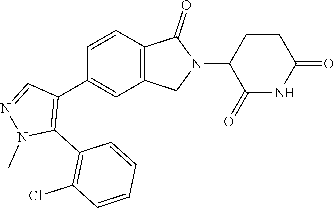 [SMILES: Cn1ncc(-c2ccc3c(c2)CN(C2CCC(=O)NC2=O)C3=O)c1-c1ccccc1Cl] | 435.2 | 1 |
| 133 | 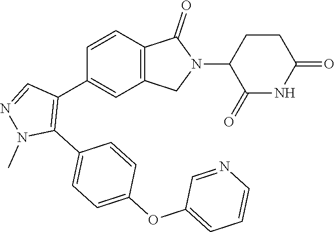 [SMILES: Cn1ncc(-c2ccc3c(c2)CN(C2CCC(=O)NC2=O)C3=O)c1-c1ccc(Oc2cccnc2)cc1] | 494.3 | 1 |
| 134 | 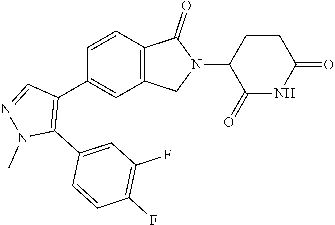 [SMILES: Cn1ncc(-c2ccc3c(c2)CN(C2CCC(=O)NC2=O)C3=O)c1-c1ccc(F)c(F)c1] | 437.2 | 1 |
| 135 | 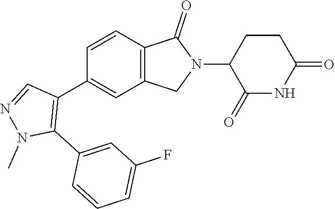 [SMILES: Cn1ncc(-c2ccc3c(c2)CN(C2CCC(=O)NC2=O)C3=O)c1-c1cccc(F)c1] | 419.2 | 1 |
| 136 | 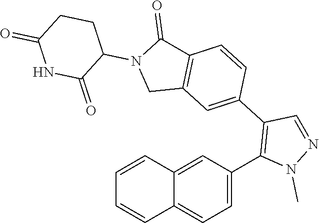 [SMILES: Cn1ncc(-c2ccc3c(c2)CN(C2CCC(=O)NC2=O)C3=O)c1-c1ccc2ccccc2c1] | 451.3 | 1 |
| 137 | 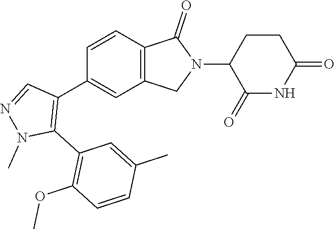 [SMILES: COc1ccc(C)cc1-c1c(-c2ccc3c(c2)CN(C2CCC(=O)NC2=O)C3=O)cnn1C] | 445.3 | 1 |
| 138 | 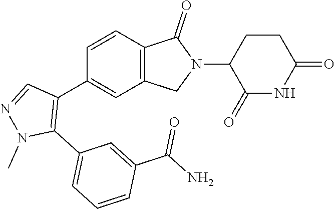 [SMILES: Cn1ncc(-c2ccc3c(c2)CN(C2CCC(=O)NC2=O)C3=O)c1-c1cccc(C(N)=O)c1] | 444.2 | 1 |
| 139 | 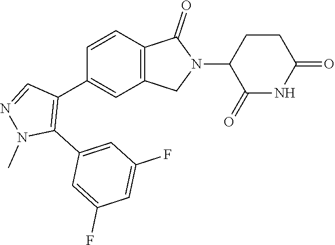 [SMILES: Cn1ncc(-c2ccc3c(c2)CN(C2CCC(=O)NC2=O)C3=O)c1-c1cc(F)cc(F)c1] | 437.2 | 1 |
| 140 | 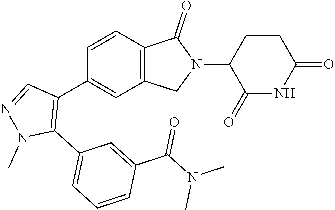 [SMILES: CN(C)C(=O)c1cccc(-c2c(-c3ccc4c(c3)CN(C3CCC(=O)NC3=O)C4=O)cnn2C)c1] | 472.3 | 1 |
| 141 | 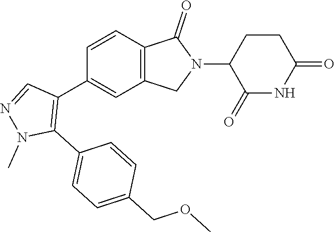 [SMILES: COCc1ccc(-c2c(-c3ccc4c(c3)CN(C3CCC(=O)NC3=O)C4=O)cnn2C)cc1] | 445.2 | 1 |
| 142 | 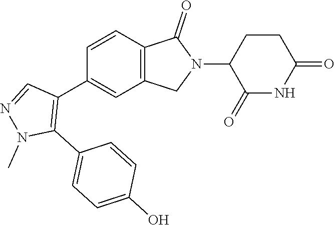 [SMILES: Cn1ncc(-c2ccc3c(c2)CN(C2CCC(=O)NC2=O)C3=O)c1-c1ccc(O)cc1] | 417.2 | 1 |
| 143 | 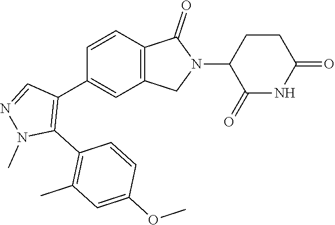 [SMILES: COc1ccc(-c2c(-c3ccc4c(c3)CN(C3CCC(=O)NC3=O)C4=O)cnn2C)c(C)c1] | 445.3 | 1 |
| 144 | 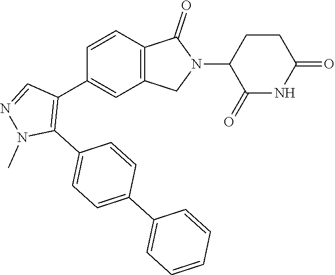 [SMILES: Cn1ncc(-c2ccc3c(c2)CN(C2CCC(=O)NC2=O)C3=O)c1-c1ccc(-c2ccccc2)cc1] | 477.3 | 1 |
| 145 | 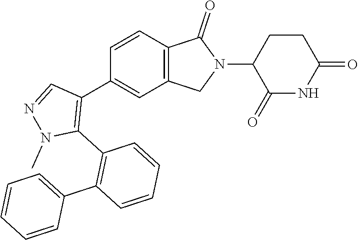 [SMILES: Cn1ncc(-c2ccc3c(c2)CN(C2CCC(=O)NC2=O)C3=O)c1-c1ccccc1-c1ccccc1] | 477.2 | 1 |
| 146 | 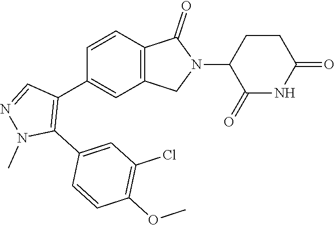 [SMILES: COc1ccc(-c2c(-c3ccc4c(c3)CN(C3CCC(=O)NC3=O)C4=O)cnn2C)cc1Cl] | 465.2 | 1 |
| 147 | 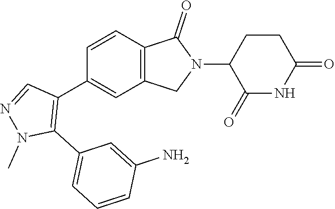 [SMILES: Cn1ncc(-c2ccc3c(c2)CN(C2CCC(=O)NC2=O)C3=O)c1-c1cccc(N)c1] | 416.2 | 1 |
| 148 | 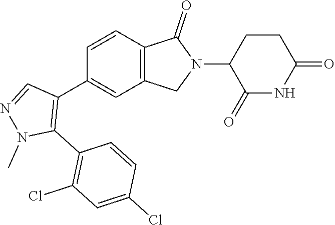 [SMILES: Cn1ncc(-c2ccc3c(c2)CN(C2CCC(=O)NC2=O)C3=O)c1-c1ccc(Cl)cc1Cl] | 469.2 | 1 |
| 149 | 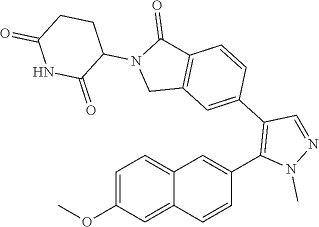 [SMILES: COc1ccc2cc(-c3c(-c4ccc5c(c4)CN(C4CCC(=O)NC4=O)C5=O)cnn3C)ccc2c1] | 481.3 | 1 |
| 150 | 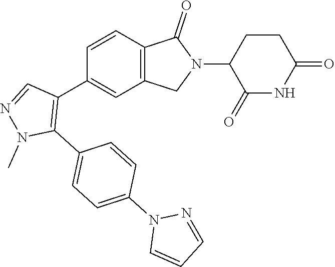 [SMILES: Cn1ncc(-c2ccc3c(c2)CN(C2CCC(=O)NC2=O)C3=O)c1-c1ccc(-n2cccn2)cc1] | 467.3 | 1 |
| 151 | 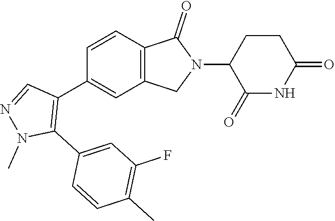 [SMILES: Cc1ccc(-c2c(-c3ccc4c(c3)CN(C3CCC(=O)NC3=O)C4=O)cnn2C)cc1F] | 433.2 | 1 |
| 152 | 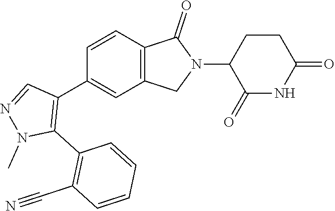 [SMILES: Cn1ncc(-c2ccc3c(c2)CN(C2CCC(=O)NC2=O)C3=O)c1-c1ccccc1C#N] | 426.3 | 1 |
| 153 | 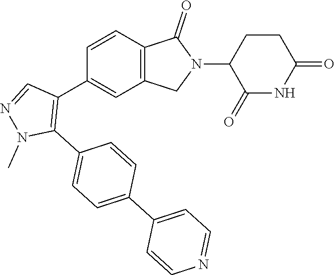 [SMILES: Cn1ncc(-c2ccc3c(c2)CN(C2CCC(=O)NC2=O)C3=O)c1-c1ccc(-c2ccncc2)cc1] | 478.3 | 1 |
| 154 | 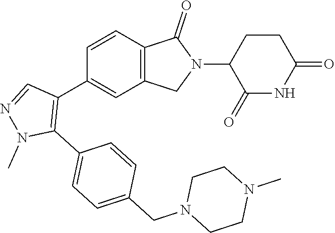 [SMILES: CN1CCN(Cc2ccc(-c3c(-c4ccc5c(c4)CN(C4CCC(=O)NC4=O)C5=O)cnn3C)cc2)CC1] | 513.4 | 1 |
| 155 | 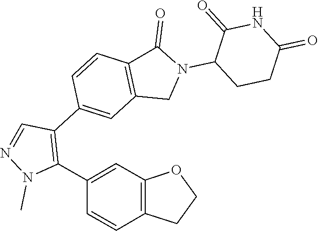 [SMILES: Cn1ncc(-c2ccc3c(c2)CN(C2CCC(=O)NC2=O)C3=O)c1-c1ccc2c(c1)OCC2] | 443.3 | 1 |
| 156 | 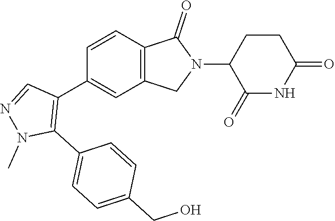 [SMILES: Cn1ncc(-c2ccc3c(c2)CN(C2CCC(=O)NC2=O)C3=O)c1-c1ccc(CO)cc1] | 431.3 | 1 |
| 157 | 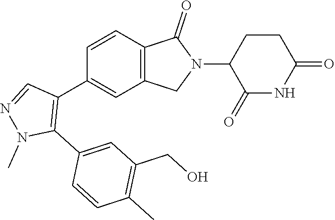 [SMILES: Cc1ccc(-c2c(-c3ccc4c(c3)CN(C3CCC(=O)NC3=O)C4=O)cnn2C)cc1CO] | 445.3 | 1 |
| 158 | 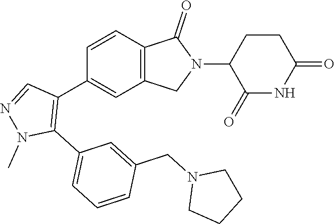 [SMILES: Cn1ncc(-c2ccc3c(c2)CN(C2CCC(=O)NC2=O)C3=O)c1-c1cccc(CN2CCCC2)c1] | 484.3 | 1 |
| 159 | 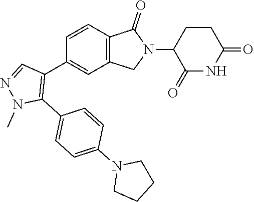 [SMILES: Cn1ncc(-c2ccc3c(c2)CN(C2CCC(=O)NC2=O)C3=O)c1-c1ccc(N2CCCC2)cc1] | 470.3 | 1 |
| 160 | 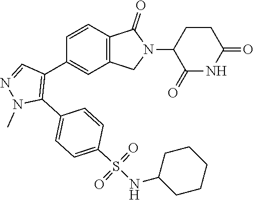 [SMILES: Cn1ncc(-c2ccc3c(c2)CN(C2CCC(=O)NC2=O)C3=O)c1-c1ccc(S(=O)(=O)NC2CCCCC2)cc1] | 562.3 | 1 |

### Examples 161-204

The compounds of Example 161-204 were prepared according to the following

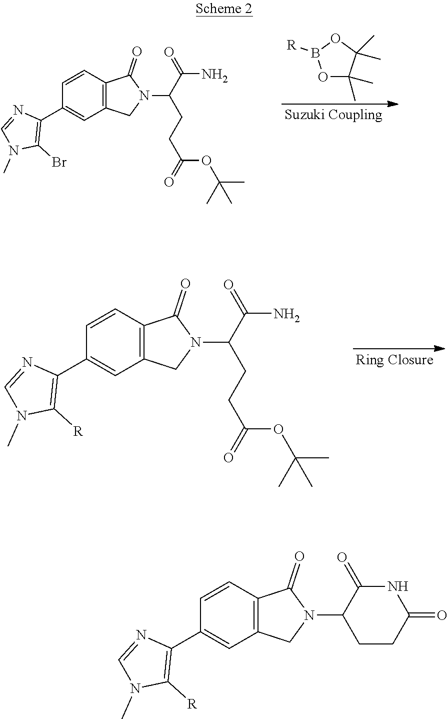

Step 1 Suzuki Coupling

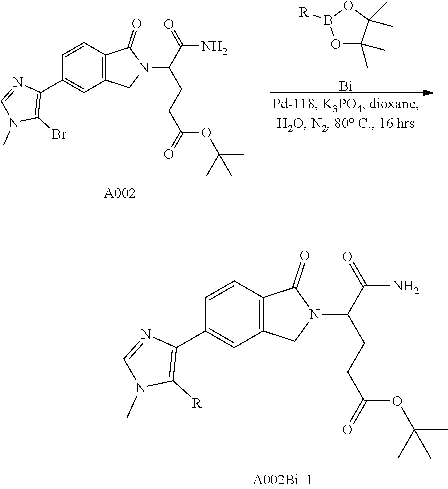

Conditions: To a vial containing a solution of A002 (100 μmol, 1.00 eq) and Bi (120 μmol, 1.20 eq) in Dioxane (0.80 mL) was added K₃PO₄ (1.5 M in H₂O, 300 μmol, 3.00 eq), and Pd-118 (10.0 μmol, 0.10 eq) under protection of N₂. The mixture was stirred at 80° C. for 16 hrs. The reaction mixture was diluted with H₂O (2.00 mL) and extracted with ethyl acetate (2.00 mL×3). The combined organic layers were washed with brine (2.00 mL), dried over Na₂SO₄, filtered and concentrated under reduced pressure to give crude intermediates used for next step.

Step 2 Ring Closure

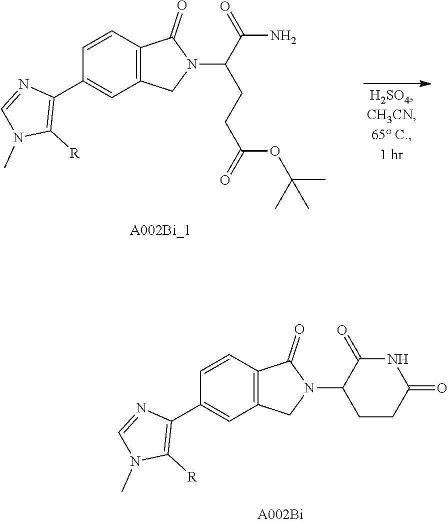

Conditions: To a vial containing a solution of A002Bi_1 (˜100 μmol, 1.00 eq) in CH₃CN (1.00 mL), was added H₂SO₄ (100 μL). The mixture was stirred at 65° C. for 1 hr. The residue was concentrated under reduced pressure and purified by prep-HPLC to give final product.

| | | MW + 1 |
|---|---|---|
| Example | Structure | (Observed) |
| 161 | 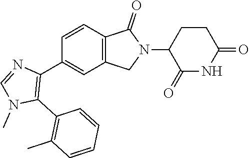 [SMILES: Cc1ccccc1-c1c(-c2ccc3c(c2)CN(C2CCC(=O)NC2=O)C3=O)ncn1C] | 415.4 |
| 162 | 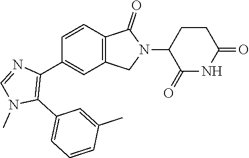 [SMILES: Cc1cccc(-c2c(-c3ccc4c(c3)CN(C3CCC(=O)NC3=O)C4=O)ncn2C)c1] | 415.3 |
| 163 | 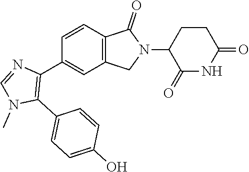 [SMILES: Cn1cnc(-c2ccc3c(c2)CN(C2CCC(=O)NC2=O)C3=O)c1-c1ccc(O)cc1] | 417.2 |
| 164 | 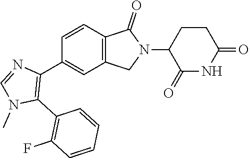 [SMILES: Cn1cnc(-c2ccc3c(c2)CN(C2CCC(=O)NC2=O)C3=O)c1-c1ccccc1F] | 419.3 |
| 165 | 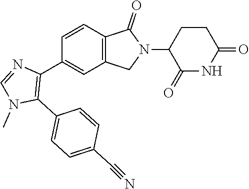 [SMILES: Cn1cnc(-c2ccc3c(c2)CN(C2CCC(=O)NC2=O)C3=O)c1-c1ccc(C#N)cc1] | 426.2 |
| 166 | 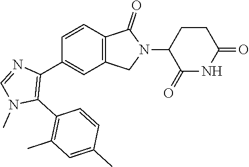 [SMILES: Cc1ccc(-c2c(-c3ccc4c(c3)CN(C3CCC(=O)NC3=O)C4=O)ncn2C)c(C)c1] | 429.3 |
| 167 | 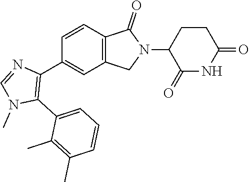 [SMILES: Cc1cccc(-c2c(-c3ccc4c(c3)CN(C3CCC(=O)NC3=O)C4=O)ncn2C)c1C] | 429.3 |
| 168 | 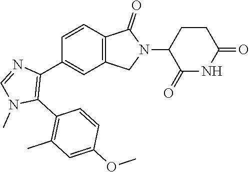 [SMILES: COc1ccc(-c2c(-c3ccc4c(c3)CN(C3CCC(=O)NC3=O)C4=O)ncn2C)c(C)c1] | 431.3 |
| 169 | 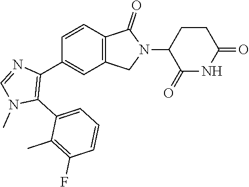 [SMILES: Cc1c(F)cccc1-c1c(-c2ccc3c(c2)CN(C2CCC(=O)NC2=O)C3=O)ncn1C] | 433.3 |
| 170 | 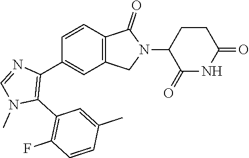 [SMILES: Cc1ccc(F)c(-c2c(-c3ccc4c(c3)CN(C3CCC(=O)NC3=O)C4=O)ncn2C)c1] | 433.3 |
| 171 | 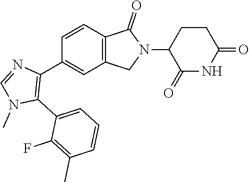 [SMILES: Cc1cccc(-c2c(-c3ccc4c(c3)CN(C3CCC(=O)NC3=O)C4=O)ncn2C)c1F] | 433.2 |
| 172 | 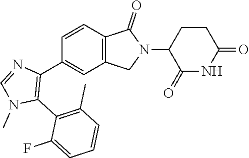 [SMILES: Cc1cccc(F)c1-c1c(-c2ccc3c(c2)CN(C2CCC(=O)NC2=O)C3=O)ncn1C] | 433.2 |
| 173 | 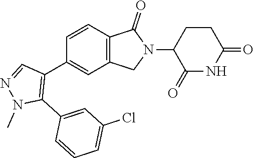 [SMILES: Cn1ncc(-c2ccc3c(c2)CN(C2CCC(=O)NC2=O)C3=O)c1-c1cccc(Cl)c1] | 435.2 |
| 174 | 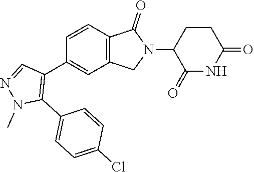 [SMILES: Cn1ncc(-c2ccc3c(c2)CN(C2CCC(=O)NC2=O)C3=O)c1-c1ccc(Cl)cc1] | 435.2 |
| 175 | 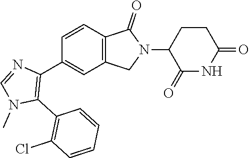 [SMILES: Cn1cnc(-c2ccc3c(c2)CN(C2CCC(=O)NC2=O)C3=O)c1-c1ccccc1Cl] | 435.2 |
| 176 | 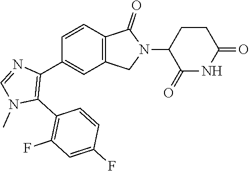 [SMILES: Cn1cnc(-c2ccc3c(c2)CN(C2CCC(=O)NC2=O)C3=O)c1-c1ccc(F)cc1F] | 437.2 |
| 177 | 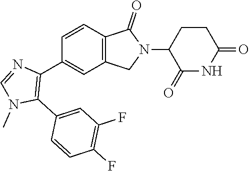 [SMILES: Cn1cnc(-c2ccc3c(c2)CN(C2CCC(=O)NC2=O)C3=O)c1-c1ccc(F)c(F)c1] | 437.2 |
| 178 | 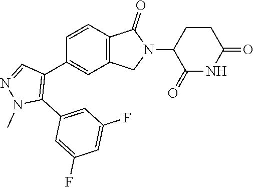 [SMILES: Cn1ncc(-c2ccc3c(c2)CN(C2CCC(=O)NC2=O)C3=O)c1-c1cc(F)cc(F)c1] | 437.2 |
| 179 | 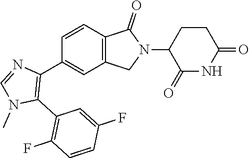 [SMILES: Cn1cnc(-c2ccc3c(c2)CN(C2CCC(=O)NC2=O)C3=O)c1-c1cc(F)ccc1F] | 437.2 |
| 180 | 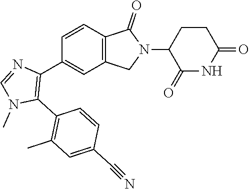 [SMILES: Cc1cc(C#N)ccc1-c1c(-c2ccc3c(c2)CN(C2CCC(=O)NC2=O)C3=O)ncn1C] | 440.3 |
| 181 | 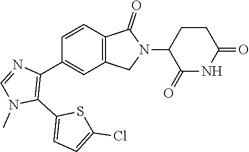 [SMILES: Cn1cnc(-c2ccc3c(c2)CN(C2CCC(=O)NC2=O)C3=O)c1-c1ccc(Cl)s1] | 441.2 |
| 182 | 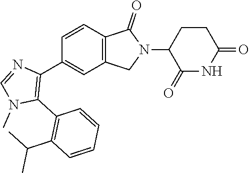 [SMILES: CC(C)c1ccccc1-c1c(-c2ccc3c(c2)CN(C2CCC(=O)NC2=O)C3=O)ncn1C] | 443.3 |
| 183 | 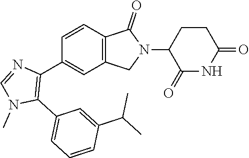 [SMILES: CC(C)c1cccc(-c2c(-c3ccc4c(c3)CN(C3CCC(=O)NC3=O)C4=O)ncn2C)c1] | 443.3 |
| 184 | 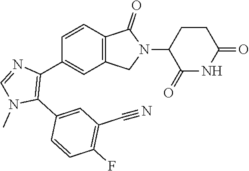 [SMILES: Cn1cnc(-c2ccc3c(c2)CN(C2CCC(=O)NC2=O)C3=O)c1-c1ccc(F)c(C#N)c1] | 444.3 |
| 185 | 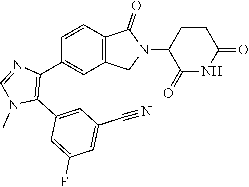 [SMILES: Cn1cnc(-c2ccc3c(c2)CN(C2CCC(=O)NC2=O)C3=O)c1-c1cc(F)cc(C#N)c1] | 444.3 |
| 186 | 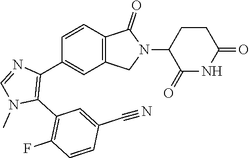 [SMILES: Cn1cnc(-c2ccc3c(c2)CN(C2CCC(=O)NC2=O)C3=O)c1-c1cc(C#N)ccc1F] | 444.2 |
| 187 | 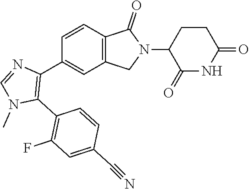 [SMILES: Cn1cnc(-c2ccc3c(c2)CN(C2CCC(=O)NC2=O)C3=O)c1-c1ccc(C#N)cc1F] | 444.1 |
| 188 | 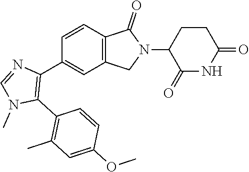 [SMILES: COc1ccc(-c2c(-c3ccc4c(c3)CN(C3CCC(=O)NC3=O)C4=O)ncn2C)c(C)c1] | 445.3 |
| 189 | 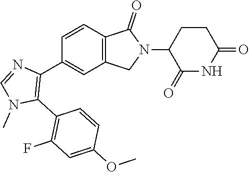 [SMILES: COc1ccc(-c2c(-c3ccc4c(c3)CN(C3CCC(=O)NC3=O)C4=O)ncn2C)c(F)c1] | 449.2 |
| 190 | 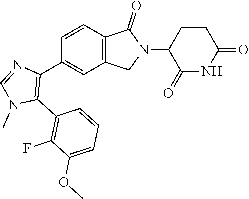 [SMILES: COc1cccc(-c2c(-c3ccc4c(c3)CN(C3CCC(=O)NC3=O)C4=O)ncn2C)c1F] | 449.2 |
| 191 | 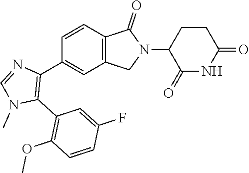 [SMILES: COc1ccc(F)cc1-c1c(-c2ccc3c(c2)CN(C2CCC(=O)NC2=O)C3=O)ncn1C] | 449.2 |
| 192 | 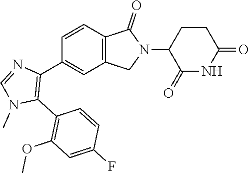 [SMILES: COc1cc(F)ccc1-c1c(-c2ccc3c(c2)CN(C2CCC(=O)NC2=O)C3=O)ncn1C] | 449.2 |
| 193 | 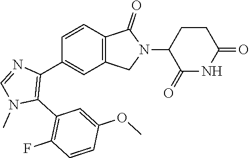 [SMILES: COc1ccc(F)c(-c2c(-c3ccc4c(c3)CN(C3CCC(=O)NC3=O)C4=O)ncn2C)c1] | 449.3 |
| 194 | 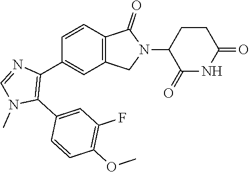 [SMILES: COc1ccc(-c2c(-c3ccc4c(c3)CN(C3CCC(=O)NC3=O)C4=O)ncn2C)cc1F] | 449.2 |
| 195 | 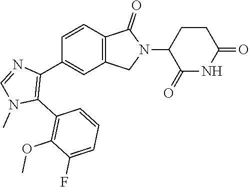 [SMILES: COc1c(F)cccc1-c1c(-c2ccc3c(c2)CN(C2CCC(=O)NC2=O)C3=O)ncn1C] | 449.2 |
| 196 | 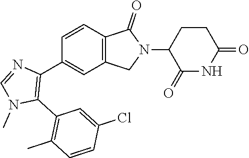 [SMILES: Cc1ccc(Cl)cc1-c1c(-c2ccc3c(c2)CN(C2CCC(=O)NC2=O)C3=O)ncn1C] | 449.2 |
| 197 | 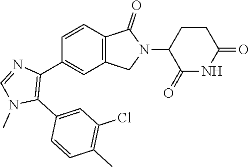 [SMILES: Cc1ccc(-c2c(-c3ccc4c(c3)CN(C3CCC(=O)NC3=O)C4=O)ncn2C)cc1Cl] | 449.2 |
| 198 | 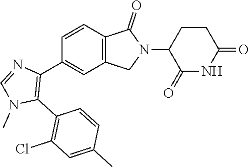 [SMILES: Cc1ccc(-c2c(-c3ccc4c(c3)CN(C3CCC(=O)NC3=O)C4=O)ncn2C)c(Cl)c1] | 449.2 |
| 199 | 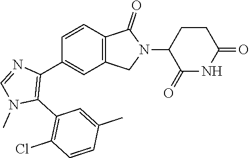 [SMILES: Cc1ccc(Cl)c(-c2c(-c3ccc4c(c3)CN(C3CCC(=O)NC3=O)C4=O)ncn2C)c1] | 449.2 |
| 200 | 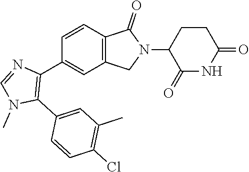 [SMILES: Cc1cc(-c2c(-c3ccc4c(c3)CN(C3CCC(=O)NC3=O)C4=O)ncn2C)ccc1Cl] | 449.2 |
| 201 | 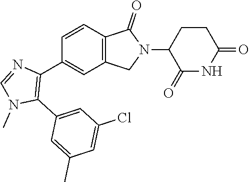 [SMILES: Cc1cc(Cl)cc(-c2c(-c3ccc4c(c3)CN(C3CCC(=O)NC3=O)C4=O)ncn2C)c1] | 449.2 |
| 202 | 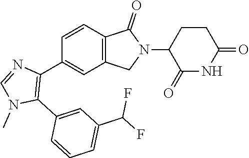 [SMILES: Cn1cnc(-c2ccc3c(c2)CN(C2CCC(=O)NC2=O)C3=O)c1-c1cccc(C(F)F)c1] | 451.2 |
| 203 | 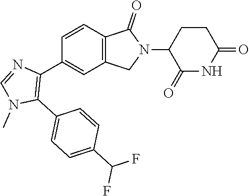 [SMILES: Cn1cnc(-c2ccc3c(c2)CN(C2CCC(=O)NC2=O)C3=O)c1-c1ccc(C(F)F)cc1] | 451.3 |
| 204 | 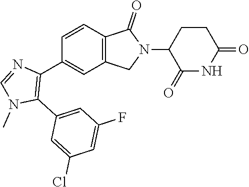 [SMILES: Cn1cnc(-c2ccc3c(c2)CN(C2CCC(=O)NC2=O)C3=O)c1-c1cc(F)cc(Cl)c1] | 453.2 |

### Example 205

### Synthesis of 3-(1-Oxo-5-(1-phenyl-1H-imidazol-5-yl)isoindolin-2-yl)piperidine-2,6-dione

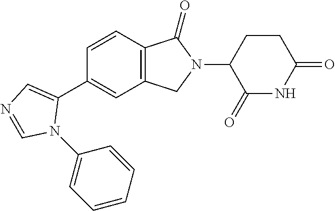 [SMILES: O=C1CCC(N2Cc3cc(-c4cncn4-c4ccccc4)ccc3C2=O)C(=O)N1]

A. (1H-Imidazol-4-yl)(phenyl)-l3-iodanyl acetate: To a solution of PhI(OAc)₂ (1.18 g, 3.67 mmol, 0.50 eq) in MeOH (5.00 mL). The mixture was stirred at 25° C. Then 1H-imidazole (500 mg, 7.34 mmol, 1.00 eq) was added into the mixture. The mixture was stirred at 25° C. for 16 h. The reaction mixture was concentrated under reduced pressure to get residue. The residue was triturated with DCM (5.00 mL) and MTBE (5.00 mL) at 25° C. for 20 min to give the title compound (1.00 g, 3.03 mmol, 41.2% yield, 96.5% purity in LCMS at 220 nm) as white solid. ¹H NMR: (400 MHz, MeOD) δ 8.07-8.05 (m, 2H), 8.05-8.04 (m, 1H), 7.84-7.84 (m, 1H), 7.66-7.63 (m, 1H), 7.52-7.48 (m, 2H), 1.89 (s, 3H). (ESI⁺) m/z: 330.1 (M+H)⁺, (C₁₁H₁₁IN₂O₂).

B. 5-Iodo-1-phenyl-1H-imidazole: To a solution of Cu(OTf)₂ (33.6 mg, 254 μmol, 0.20 eq) and Cs₂CO₃ (621 mg, 1.91 mmol, 1.50 eq) in HFIP (4.00 mL) were added N-methylbenzimidazole (23.0 mg, 63.6 μmol, 0.05 eq). The mixture was stirred at 25° C. for 30 min. Then (1H-imidazol-4-yl) (phenyl)-l3-iodaneyl acetate (420 mg, 1.27 mmol, 1.00 eq) was added into the mixture. Then the mixture was stirred at 50° C. for 16 h. The reaction mixture was concentrated under reduced pressure to get residue. The residue was purified by preparative-TLC (Petroleum ether:Ethyl acetate=3:1, R_{f}=0.30) to give the title compound (200 mg, 621 μmol, 48.8% yield, 89.3.0% purity in LCMS at 220 nm) as yellow solid. (ESI⁺) m/z: 270.9 (M+H)⁺, (C₉H₇IN₂).

C. 3-(1-Oxo-5-(1-phenyl-1H-imidazol-5-yl)isoindolin-2-yl)piperidine-2,6-dione: To a solution of 5-iodo-1-phenyl-1H-imidazole (200 mg, 740 μmol, 1.00 eq), 3-(1-oxo-5-(4,4,5,5-tetramethyl-1,3,2-dioxaborolan-2-yl)isoindolin-2-yl)piperidine-2,6-dione (822 mg, 2.21 mmol, 3.00 eq) and K₃PO₄ (471 mg, 2.21 mmol, 3.00 eq) in dioxane (5.00 mL) and H₂O (0.25 mL) was added Ru-Phos-Pd-G₃ (123 mg, 147 μmol, 0.20 eq) under N₂. The mixture was stirred at 100° C. for 6 h under N₂. The reaction mixture was concentrated under reduced pressure to get a residue at 43° C. The residue was purified by preparative-HPLC (using a Phenomenex luna C18 (150×25 mm×5 μm) and gradient of 5.00%-35.0% acetonitrile in water containing 0.50% FA over 10 min at a flow rate of 25 mL/min) to give the title compound (23.6 mg, 61.1 μmol, 7.19% yield, 100% purity in HPLC at 220 nm) as white solid. ¹H NMR: (400 MHz, DMSO-d₆) δ 11.0-10.9 (m, 1H), 8.99-8.96 (m, 1H), 7.92-7.89 (m, 1H), 7.68-7.66 (m, 1H), 7.53-7.42 (m, 6H), 7.28-7.23 (m, 1H), 5.11-5.07 (m, 1H), 4.45-4.29 (m, 2H), 2.96-2.85 (m, 1H), 2.60-2.53 (m, 1H), 2.40-2.39 (m, 1H), 2.00-1.97 (m, 1H). (ESI⁺) m/z: 387.2 (M+H)⁺, (C₂₂H₁IN₄O₃).

### Example 206

### Synthesis of 3-(1-Oxo-5-(3-phenylisoxazol-4-yl)isoindolin-2-yl)piperidine-2,6-dione

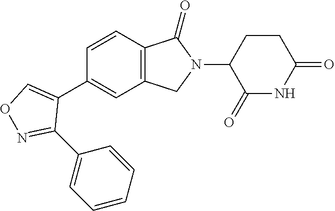 [SMILES: O=C1CCC(N2Cc3cc(-c4conc4-c4ccccc4)ccc3C2=O)C(=O)N1]

A. 3-Phenylisoxazole: To solution of ((E)-N-hydroxybenzimidoyl chloride (2.00 g, 12.8 mmol, 1.00 eq) and ethynyltrimethylsilane (1.39 g, 14.1 mmol, 1.96 mL, 1.10 eq) in DCM (24.0 mL) was added TEA (3.25 g, 32.1 mmol, 4.47 mL, 2.50 eq) at 0° C. under N₂. After being stirred for 1.5 h at 50° C. The reaction mixture was diluted with DCM (3×30.0 mL) and the organic phase was washed with water (30.0 mL) and brine (2×30.0 mL), dried over Na₂SO₄, and concentrated under reduced pressure to get residue. The residue was dissolved in EtOH (50.0 mL) and CsF (11.7 g, 77.1 mmol, 2.85 mL, 6.00 eq) was added under N₂. Then the mixture was stirred at 20° C. for 1.5 h under N₂. The reaction mixture was poured into H₂O (50.0 mL) and extracted with DCM (3×60.0 mL). The combined organic layer was washed with saturated NaCl aqueous (2×50.0 mL), dried over Na₂SO₄ and concentrated under reduced pressure to get residue. The residue was purified by column chromatography (SiO₂, Petroleum ether:Ethyl acetate=10:1, R_{f}=0.40) to get the title compound (1.20 g, 7.93 mmol, 61.7% yield, 95.9% purity in LCMS at 220 nm) as colorless oil. (ESI⁺) m/z: 146.0 (M+H)⁺, (C₉H₇NO).

B. 4-Iodo-3-phenylisoxazole: To a solution of 3-phenylisoxazole (350 mg, 2.41 mmol, 1.00 eq) in TFA (4.00 mL) was added NIS (488 mg, 2.17 mmol, 0.90 eq) under N₂. The reaction mixture was stirred at 50° C. for 8 h under N₂. The mixture was poured into saturated NaHCO₃ aqueous (40.0 mL), extracted with Ethyl acetate (3×30.0 mL). The combined organic layer was washed with 10% Na₂S₂O₃ solution (2×30.0 mL) and brine (2×20.0 mL), dried over Na₂SO₄ and concentrated under reduced pressure to get residue. The residue was purified by preparative-TLC (Petroleum ether/Ethyl acetate=10/1, R_{f}=0.35) to give the title compound (340 mg, 1.13 mmol, 46.7% yield, 89.8% purity in LCMS at 220 nm) as white solid. (ESI⁺) m/z: 272.0 (M+H)⁺, (C₉H₆INO).

C. 3-(1-Oxo-5-(3-phenylisoxazol-4-yl)isoindolin-2-yl)piperidine-2,6-dione: To a solution of 4-iodo-3-phenylisoxazole (270 mg, 996 μmol, 1.00 eq), 3-(1-oxo-5-(4,4,5,5-tetramethyl-1,3,2-dioxaborolan-2-yl)isoindolin-2-yl)piperidine-2,6-dione (553 mg, 1.49 mmol, 1.50 eq) and K₃PO₄ (422 mg, 1.99 mmol, 2.00 eq) in dioxane (6.00 mL) and H₂O (0.30 mL) was added Ru-Phos-Pd-G₃ (167 mg, 199 μmol, 0.20 eq) under N₂. The mixture was stirred at 100° C. for 3 h under N₂. The mixture was filtered to collect liquid and concentrated under reduced pressure to get residue. The crude product was purified by preparative-HPLC (using a Phenomenex luna C18 (150 mm×25 mm×10 μm) and gradient of 24-54% acetonitrile in water containing 0.05% FA over 15 min at a flow rate of 25 mL/min) to give the title compound (129 mg, 323 μmol, 32.4% yield, 97.0% purity in HPLC at 220 nm) as white solid. ¹H NMR: (400 MHz, DMSO-d₆) δ 10.9 (s, 1H), 9.36 (s, 1H), 7.73-7.71 (m, 1H), 7.54-7.51 (m, 1H), 7.49-7.45 (m, 5H), 7.38-7.36 (m, 1H), 5.14-5.09 (m, 1H), 4.45-4.28 (m, 2H), 2.94-2.87 (m, 1H), 2.60-2.57 (m, 1H), 2.41-2.38 (m, 1H), 2.02-1.99 (m, 1H). (ESI⁺) m/z: 388.0 (M+H)⁺, (C₂₂H₁₇N₃O₄).

### Example 207

### Synthesis of 3-(5-(1-(Ethyl-d₃)-5-phenyl-1H-pyrazol-4-yl)-1-oxoisoindolin-2-yl)piperidine-2,6-dione

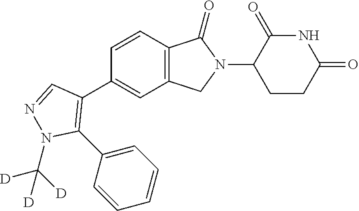 [SMILES: [2H]C([2H])([2H])n1ncc(-c2ccc3c(c2)CN(C2CCC(=O)NC2=O)C3=O)c1-c1ccccc1]

A. 4-Bromo-1-(methyl-d₃)-1H-pyrazole: A mixture of 4-bromo-1H-pyrazole (6.00 g, 40.8 mmol, 1.00 eq) and trideuterio(iodo)methane (9.27 g, 65.3 mmol, 3.98 mL, 1.60 eq), t-BuOK (9.16 g, 81.7 mmol, 2.00 eq) in THF (60.0 mL) was stirred at 25° C. for 4 h. The reaction mixture was poured into 50.0 mL H₂O, extracted with EtOAc (3×50.0 mL), then concentrated to give a residue to give the title compound (4.00 g, 24.4 mmol, 59.7% yield) as colorless oil. ¹H NMR: (400 MHz, DMSO-d₆) δ 7.92 (s, 1H), 7.51 (s, 1H). (ESI⁺) m/z: 162.9 (M+H)⁺ (C₄H₂D₃BrN₂).

B. 4-Bromo-1-(methyl-d₃)-5-phenyl-1H-pyrazole: To a solution of 4-bromo-1-(methyl-d₃)-1H-pyrazole (400 mg, 2.03 mmol, 1.00 eq) and bromobenzene (956 mg, 6.09 mmol, 642 μL, 3.00 eq) in DMF (4.00 mL) was added Pd(OAc)₂ (45.6 mg, 203 μmol, 0.10 eq), P(oxole)₃ (94.3 mg, 406 μmol, 0.20 eq) and K₂CO₃ (561 mg, 4.06 mmol, 2.00 eq) under N₂. The reaction mixture was stirred at 110° C. for 48 h under N₂. The reaction mixture was poured into H₂O (20.0 mL) and extracted with Ethyl acetate (3×20.0 mL). Then the organic layer was dried by Na₂SO₄, filtered and concentrated to get residue. The residue was purified by column chromatography (SiO₂, Petroleum ether:Ethyl acetate=50:1 to 10:1, TLC: Petroleum ether:Ethyl acetate=3:1,R_{f}=0.30) to give the title compound (250 mg, 1.04 mmol, 42.3% yield, 95.2% purity in LCMS at 220 nm) as yellow oil. ¹H NMR: (400 MHz, CD₃Cl) δ 7.55 (s, 1H), 7.52-7.49 (m, 3H), 7.43-7.41 (m, 2H), (ESI⁺) m/z: 241.7 (M+H)⁺, (C₁₀H₆D₃BrN₂).

C. 3-(5-(1-(Methyl-d₃)-5-phenyl-1H-pyrazol-4-yl)-1-oxoisoindolin-2-yl)piperidine-2,6-dione: To a solution of 4-bromo-1-(methyl-d₃)-5-phenyl-1H-pyrazole (140 mg, 583 μmol, 1.00 eq), 3-(1-oxo-5-(4,4,5,5-tetramethyl-1,3,2-dioxaborolan-2-yl)isoindolin-2-yl)piperidine-2,6-dione (647 mg, 1.75 mmol, 3.00 eq) and K₃PO₄ (371.0 mg, 1.75 mmol, 3.00 eq) in dioxane (7.00 mL) and H₂O (0.35 mL) was added Ru-Phos-Pd-G₃ (48.7 mg, 58.3 μmol, 0.10 eq) under N₂. The mixture was stirred at 100° C. for 2 h under N₂. The reaction mixture was concentrated under reduced pressure to get a residue at 45° C. The residue was purified by preparative-HPLC (using a Phenomenex luna C18 (150×25 mm×10 μm) and gradient of 22.0%-52.0% acetonitrile in water containing 0.50% TFA over 10 min at a flow rate of 25 mL/min) to give the title compound (133 mg, 339 μmol, 58.2% yield, 99.5% purity in HPLC at 220 nm) was obtained as white solid. ¹H NMR: (400 MHz, DMSO) δ 11.0-10.9 (m, 1H), 7.90 (s, 1H), 7.55-7.51 (m, 4H), 7.42-7.38 (m, 3H), 7.22-7.20 (m, 1H), 5.09-5.02 (m, 1H), 4.36-4.17 (m, 2H), 2.92-2.86 (m, 1H), 2.59-2.51 (m, 1H), 2.38-2.32 (m, 1H), 1.98-1.95 (m, 1H). Deuterated ratio: 96.8%. (ESI⁺) m/z: 404.1 (M+H)⁺, (C₂₃H₁₇D₃N₄O₃).

### Example 208

### Synthesis of 3-(1-Oxo-5-(5-phenyl-1-(2,2,2-trifluoroethyl)-1H-pyrazol-4-yl)isoindolin-2-yl)piperidine-2,6-dione

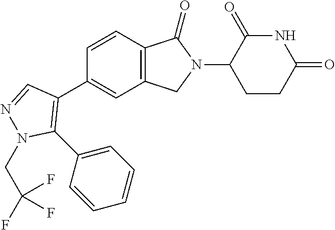 [SMILES: O=C1CCC(N2Cc3cc(-c4cnn(CC(F)(F)F)c4-c4ccccc4)ccc3C2=O)C(=O)N1]

A. 4-Bromo-1-(2,2,2-trifluoroethyl)-1H-pyrazole: To a solution of 4-bromo-1H-pyrazole (3.00 g, 20.4 mmol, 1.00 eq) and Cs₂CO₃ (19.9 g, 61.2 mmol, 3.00 eq) in DMF (10.0 mL) was added dropwise 2,2,2-trifluoroethyl trifluoromethanesulfonate (5.21 g, 22.4 mmol, 1.10 eq). The mixture was stirred at 25° C. for 2 h. Then the reaction mixture was quenched with 5.00 mL of water and filtered. The filtrate was concentrated in vacuum to get a residue. The residue was purified by column chromatography (SiO₂, Petroleum ether:EtOAc=100:1 to 10:1, R_{f}=0.40 (Petroleum ether:EtOAc=10:1)) to give the title compound (1.70 g, 7.42 mmol, 36.3% yield) as colorless oil. ¹H NMR: (400 MHz, CDCl₃) (7.56 (s, 1H), 7.55 (s, 1H), 4.68 (dd, J=16.4, 8.4 Hz, 2H). (ESI⁺) m/z: 228.9 (M+H)⁺, (C₅H₄BrF₃N₂).

B. 4-Bromo-5-phenyl-1-(2,2,2-trifluoroethyl)-1H-pyrazole: To a solution of 4-bromo-1-(2,2,2-trifluoroethyl)pyrazole (500 mg, 2.18 mmol, 1.00 eq) and bromobenzene (342 mg, 2.18 mmol, 229 μL, 1.00 eq) in NMP (7.50 mL) was added Pd(OAc)₂ (4.90 mg, 21.8 μmol, 0.01 eq), Davephos (17.1 mg, 43.6 μmol, 0.02 eq), Bu₄NOAc (1.32 g, 4.37 mmol, 1.33 mL, 2.00 eq) and isobutyric acid (57.7 mg, 655 μmol, 60.7 μL, 0.30 eq) under N₂. The mixture was stirred at 100° C. for 16 h under N₂. Then the reaction mixture was poured in to 10.0 mL of brine and extracted with EtOAc (3×10.0 mL). The combined organic layers were concentrated in vacuum to get a residue. The residue was purified by preparative-TLC (SiO₂, Petroleum ether:EtOAc=10:1, R_{f}=0.60) to give the title compound (360 mg, 821 μmol, 37.6% yield, 69.5% purity in LCMS at 220 nm) as colorless oil. ¹H NMR: (400 MHz, CDCl₃) (7.68 (s, 1H), 7.56-7.51 (m, 3H), 7.40-7.35 (m, 2H), 4.69 (dd, J=16.8, 8.4 Hz, 2H). (ESI⁺) m/z: 304.9 (M+H)⁺, (C₁₁H₈BrF₃N₂).

C. tert-Butyl 5-amino-5-oxo-4-(1-oxo-5-(5-phenyl-1-(2,2,2-trifluoroethyl)-1H-pyrazol-4-yl)isoindolin-2-yl)pentanoate: To a solution of 4-bromo-5-phenyl-1-(2,2,2-trifluoroethyl)pyrazole (350 mg, 798 μmol, 1.00 eq) and tert-butyl 5-amino-5-oxo-4-[1-oxo-5-(4,4,5,5-tetramethyl-1,3,2-dioxaborolan-2-yl)isoindolin-2-yl]pentanoate (532 mg, 1.20 mmol, 1.50 eq) in dioxane (4.00 mL) and H₂O (0.20 mL) was added K₃PO₄ (508 mg, 2.39 mmol, 3.00 eq) and Ru-Phos-Pd-G₃ (133 mg, 159 μmol, 0.20 eq). The mixture was stirred at 100° C. for 2 h under N₂. Then the reaction mixture was filtered and the filtrate was concentrated in vacuum to get a residue. The residue was purified by preparative-TLC (SiO₂, Petroleum ether:EtOAc=0:1, R_{f}=0.50) and then by reversed-phase HPLC (0.5% HCl condition) to give the title compound (300 mg, 528 μmol, 66.2% yield, 95.6% purity in LCMS at 220 nm) as a white solid. ¹H NMR: (400 MHz, CDCl₃) δ 7.99 (s, 1H), 7.79-7.72 (m, 1H), 7.64-7.53 (m, 4H), 7.44-7.36 (m, 3H), 6.47 (br, 1H), 4.94 (br, 1H), 4.72-4.63 (m, 2H), 4.53-4.32 (m, 2H), 2.51-2.19 (m, 5H), 1.48 (s, 9H). (ESI⁺) m/z: 543.1 (M+H)⁺, (C₂₈H₂₉F₃N₄O₄).

D. 3-(1-Oxo-5-(5-phenyl-1-(2,2,2-trifluoroethyl)-1H-pyrazol-4-yl)isoindolin-2-yl)piperidine-2,6-dione: A mixture of tert-butyl 5-amino-5-oxo-4-[1-oxo-5-[5-phenyl-1-(2,2,2-trifluoroethyl)pyrazol-4-yl]isoindolin-2-yl]pentanoate (150 mg, 264 μmol, 1.00 eq) and TsOH (455 mg, 2.64 mmol, 10.0 eq) in ACN (2.00 mL) was stirred at 80° C. for 3 h under N₂ atmosphere. Then the reaction mixture was poured into 10.0 mL of water and extracted with EtOAc (3×10.0 mL). The combined organic layer was washed with brine (10.0 mL), dried over Na₂SO₄ and filtered. The filtrate was concentrated in vacuum to get a residue. The residue was purified by column chromatography (SiO₂, Petroleum ether:EtOAc=100:1 to 0:1, R_{f}=0.45 (Petroleum ether:EtOAc=0:1)) and then by preparative-HPLC (using a Welch Ultimate C18 (150 mm×25 mm×5 μm) and gradient of 35-55% acetonitrile in water containing 0.5% HCl over 10 min at a flow rate of 25 mL/min) to give the title compound (28.0 mg, 59.9 μmol, 22.6% yield, 100% purity in HPLC at 220 nm) as a white solid. ¹H NMR: (400 MHz, DMSO-d₆) δ 10.9 (s, 1H), 8.11 (s, 1H), 7.59-7.51 (m, 4H), 7.42-7.35 (m, 3H), 7.25-7.20 (m, 1H), 5.07 (dd, J=13.2, 4.8 Hz, 1H), 4.90 (dd, J=17.6, 8.8 Hz, 2H), 4.27 (dd, J=57.6, 17.2 Hz, 2H), 2.95-2.84 (m, 1H), 2.60-2.54 (m, 1H), 2.43-2.35 (m, 1H), 2.00-1.93 (m, 1H). (ESI⁺) m/z: 469.1 (M+H)⁺, (C₂₄H₁₉F₃N₄O₃).

### Example 209

### Synthesis of 3-(5-(1-methyl-2-(4-(trifluoromethyl)phenyl)-1H-imidazol-4-yl)-1-oxoisoindolin-2-yl)piperidine-2,6-dione

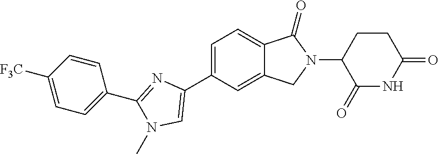 [SMILES: Cn1cc(-c2ccc3c(c2)CN(C2CCC(=O)NC2=O)C3=O)nc1-c1ccc(C(F)(F)F)cc1]

A. tert-Butyl 5-amino-4-(5-(1-methyl-2-(4-(trifluoromethyl)phenyl)-1H-imidazol-4-yl)-1-oxoisoindolin-2-yl)-5-oxopentanoate: To a solution of tert-butyl 5-amino-4-[5-(1-methylimidazol-4-yl)-1-oxo-isoindolin-2-yl]-5-oxo-pentanoate (50.0 mg, 125 μmol, 1.00 eq) and 1-iodo-4-(trifluoromethyl)benzene (40.9 mg, 150 μmol, 22.1 μL, 1.20 eq) in dioxane (2.00 mL) was added Pd(OAc)₂ (1.41 mg, 6.27 μmol, 0.05 eq), PPh₃ (3.29 mg, 12.5 μmol, 0.10 eq), DBU (38.2 mg, 250 μmol, 37.8 μL, 2.00 eq) and CuI (47.8 mg, 250 μmol, 2.00 eq). The mixture was stirred at 140° C. for 8 h under N₂. After the reaction was completed, the reaction mixture was filtered and the filtrate was concentrated in vacuum to get a residue. The residue was purified by preparative-TLC (SiO₂, Dichloromethane:Methanol=10:1, R_{f}=0.60) to give the title compound (15.0 mg, 21.1 μmol, 16.8% yield, 76.7% purity in LCMS at 220 nm) as an off-white solid. ¹H NMR: (400 MHz, CDCl₃) δ 7.98 (s, 1H), 7.90-7.81 (m, 4H), 7.76 (d, J=8.0 Hz, 2H), 7.41 (s, 1H), 6.43 (s, 1H), 5.44 (s, 1H), 4.91 (t, J=7.6 Hz, 1H), 4.54 (d, J=17.2 Hz, 1H), 4.46 (d, J=16.8 Hz, 1H), 3.83 (s, 3H), 2.39-2.30 (m, 2H), 2.26-2.16 (m, 2H), 1.42 (s, 9H). (ESI⁺) m/z: 543.1 (M+H)⁺, (C₂₈H₂₉F₃N₄O₄).

B. tert-Butyl 4-(5-(1-methyl-1H-imidazol-4-yl)-1-oxoisoindolin-2-yl)-5-oxo-5-((4-(trifluoromethyl)phenyl)amino)pentanoate: To a solution of tert-butyl 5-amino-4-[5-(1-methylimidazol-4-yl)-1-oxo-isoindolin-2-yl]-5-oxo-pentanoate (50.0 mg, 125 μmol, 1.00 eq) and 1-iodo-4-(trifluoromethyl)benzene (40.9 mg, 150 μmol, 22.1 μL, 1.20 eq) in toluene (2.00 mL) was added Pd(OAc)₂ (2.82 mg, 12.5 μmol, 0.10 eq), DPPF (3.48 mg, 6.27 μmol, 0.05 eq), CuIXantphos (28.9 mg, 37.6 μmol, 0.30 eq) and Cs₂CO₃ (122 mg, 376 μmol, 3.00 eq). The mixture was stirred at 125° C. for 8 h under N₂. After the reaction was completed, the reaction mixture was filtered and the filtrate was concentrated in vacuum to get a residue. The residue was purified by preparative-TLC (SiO₂, Dichloromethane:Methanol=10:1, R_{f}=0.65) to give the title compound (27.0 mg, 35.0 μmol, 27.9% yield, 70.5% purity in LCMS at 220 nm) as an off-white solid. ¹H NMR: (400 MHz, CDCl₃) δ 9.60 (s, 1H), 7.90 (s, 1H), 7.79-7.67 (m, 5H), 7.54-7.49 (m, 3H), 5.11 (t, J=7.6 Hz, 1H), 4.62 (d, J=17.2 Hz, 1H), 4.53 (d, J=16.8 Hz, 1H), 3.74 (s, 3H), 2.50-2.36 (m, 2H), 2.34-2.24 (m, 2H), 1.39 (s, 9H). (ESI⁺) m/z: 543.1 (M+H)⁺, (C₂₈H₂₉F₃N₄O₄).

C. tert-Butyl 5-amino-4-(5-(1-methyl-2-(4-(trifluoromethyl)phenyl)-1H-imidazol-4-yl)-1-oxoisoindolin-2-yl)-5-oxopentanoate: To a solution of tert-butyl 5-amino-4-[5-(1-methylimidazol-4-yl)-1-oxo-isoindolin-2-yl]-5-oxo-pentanoate (200 mg, 501 μmol, 1.00 eq) and 1-iodo-4-(trifluoromethyl)benzene (273 mg, 1.00 mmol, 147 μL, 2.00 eq) in toluene (8.00 mL) was added Pd(OAc)₂ (11.2 mg, 50.1 μmol, 0.10 eq), DPPF (55.6 mg, 100 μmol, 0.20 eq), CuTC (38.2 mg, 200 μmol, 0.40 eq), CsOPiv (234 mg, 1.00 mmol, 2.00 eq). The mixture was stirred at 100° C. for 16 h under N₂. After the reaction was completed, the reaction mixture was filtered and the filtrate was concentrated in vacuum to get a residue. The residue was purified by preparative-TLC (SiO₂, Dichloromethane:Methanol=10:1, R_{f}=0.60) to give the title compound (120 mg, 211 μmol, 42.0% yield, 95.5% purity in LCMS at 220 nm) as an off-white solid. (ESI⁺) m/z: 543.1 (M+H)⁺, (C₂₈H₂₉F₃N₄O₄).

D. 3-(5-(1-Methyl-2-(4-(trifluoromethyl)phenyl)-1H-imidazol-4-yl)-1-oxoisoindolin-2-yl)piperidine-2,6-dione: A mixture of tert-butyl 5-amino-4-[5-[1-methyl-2-[4-(trifluoromethyl)phenyl]imidazol-4-yl]-1-oxo-isoindolin-2-yl]-5-oxo-pentanoate (104 mg, 184 μmol, 1.00 eq) and TsOH (317 mg, 1.84 mmol, 10.0 eq) in ACN (2.00 mL) was stirred at 80° C. for 2 h under N₂. After the reaction was completed, the reaction mixture was filtered and the filtrate was concentrated in vacuum to get a residue. The residue was purified by preparative-HPLC (using a Welch Xtimate C18 (150 mm×25 mm×5 μm) and gradient of 14-44% acetonitrile in water containing 0.1% TFA over 10 min at a flow rate of 25 mL/min) to give the title compound (40.4 mg, 86.4 μmol, 46.8% yield, 100% purity in HPLC at 220 nm) as a white solid. ¹H NMR: (400 MHz, DMSO-d₆) δ 10.9 (s, 1H), 8.09-8.03 (m, 4H), 7.98-7.95 (m, 1H), 7.92 (d, J=8.4 Hz, 2H), 7.78-7.74 (m, 1H), 5.13 (dd, J=12.8, 5.2 Hz, 1H), 4.52 (d, J=17.2 Hz, 1H), 4.38 (d, J=17.6 Hz, 1H), 3.88 (s, 3H), 2.98-2.87 (m, 1H), 2.64-2.60 (m, 1H), 2.44-2.39 (m, 1H), 2.07-1.99 (m, 1H). (ESI⁺) m/z: 469.0 (M+H)⁺, (C₂₄H₁₉F₃N₄O₃).

### Example 210

### Synthesis of 3-(1-Oxo-5-(5-phenyl-1H-pyrazol-3-yl)isoindolin-2-yl)piperidine-2,6-dione

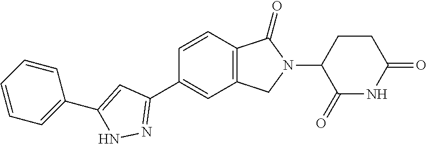 [SMILES: O=C1CCC(N2Cc3cc(-c4cc(-c5ccccc5)[nH]n4)ccc3C2=O)C(=O)N1]

A. 5-Phenyl-1-(tetrahydro-2H-pyran-2-yl)-1H-pyrazole: To a solution of 5-phenyl-1H-pyrazole (10.0 g, 69.4 mmol, 1.00 eq) in Tol. (70.0 mL) was added TFA (791 mg, 6.94 mmol, 515 μL, 0.10 eq). Then DHP (6.42 g, 76.3 mmol, 6.98 mL, 1.10 eq) was added to the mixture at 80° C. The reaction mixture was stirred at 80° C. for 12 h. The solvent was removed under reduced pressure to get residue. Then the residue was poured into H₂O (150 mL) and extracted with ethyl acetate (3×100 mL). The combined organic layer was washed with saturated NaCl aqueous (3×100 mL), dried over by Na₂SO₄, and concentrated under reduced pressure. The residue was purified by column chromatography (SiO₂, Petroleum ether:Ethyl acetate=2:1, R_{f}=0.50) to get title compound (14.8 g, 64.8 mmol, 93.4% yield) as light yellow oil. ¹H NMR: (400 MHz, DMSO-d₆) δ 7.92 (s, 1H), 7.91-7.79 (m, 2H), 7.41-7.37 (m, 2H), 7.31-7.29 (m, 1H), 6.76 (s, 1H), 5.44-5.41 (m, 1H), 3.95-3.92 (m, 1H), 3.67-3.60 (m, 1H), 2.16-2.11 (m, 1H), 1.96-1.92 (m, 2H), 1.56-1.52 (m, 3H). (ESI⁺) m/z: 229.29 (M+H)⁺, (C₁₄H₁₆N₂O).

B. 3-Bromo-5-phenyl-1-(tetrahydro-2H-pyran-2-yl)-1H-pyrazole: To a solution of 5-phenyl-1-(tetrahydro-2H-pyran-2-yl)-1H-pyrazole (7.00 g, 30.6 mmol, 1.00 eq) in THF (70.0 mL) was cooled to −70° C., then n-BuLi (2.5 M, 13.5 mL, 1.10 eq) was dropwise to the mixture at −70° C. under N₂. Then the reaction mixture was stirred at −70° C. for 1 h, after that a solution of (CF₂Br)₂ (8.76 g, 33.7 mmol, 1.10 eq) in THF (70.0 mL) was added to the reaction mixture at −70° C. under N₂. The mixture was stirred at 20° C. for 3 h under N₂. The mixture was poured into ice H₂O (300 mL) and extracted with Ethyl acetate (3×200 mL). The combined organic layer was washed with saturated NaCl aqueous (3×150 mL), dried over Na₂SO₄ and concentrated under reduced pressure to get title compound (3.50 g, 11.4 mmol, 37.1% yield) as yellow oil. ¹H NMR: (400 MHz, DMSO-d₆) δ 7.82-7.80 (m, 2H), 7.44-7.40 (m, 2H), 7.36-7.32 (m, 1H), 7.05 (s, 1H), 5.50-5.47 (m, 1H), 3.95-3.92 (m, 1H), 3.66-3.60 (m, 1H), 2.38-2.35 (m, 1H), 2.02-1.93 (m, 1H), 1.91-1.90 (m, 1H), 1.74-1.70 (m, 1H), 1.58-1.53 (m, 2H). (ESI⁺) m/z: 223.0 (M-84)⁺, (C₁₄H₁₅BrN₂O).

C. 3-(1-Oxo-5-(5-phenyl-1-(tetrahydro-2H-pyran-2-yl)-1H-pyrazol-3-yl)isoindolin-2-yl)piperidine-2,6-dione: To a solution of 3-bromo-5-phenyl-1-(tetrahydro-2H-pyran-2-yl)-1H-pyrazole (500 mg, 1.63 mmol, 1.00 eq), 3-(1-oxo-5-(4,4,5,5-tetramethyl-1,3,2-dioxaborolan-2-yl)isoindolin-2-yl)piperidine-2,6-dione (1.51 g, 4.07 mmol, 2.50 eq) and K₃PO₄ (691 mg, 3.26 mmol, 2.00 eq) in dioxane (10.0 mL) and H₂O (0.50 mL) was added Ru-Phos-Pd-G₃ (272 mg, 325 μmol, 0.20 eq) under N₂. The mixture was stirred at 100° C. for 2 h under N₂. The mixture was filtered to collect liquid and concentrated under reduced pressure to get residue. The residue was purified by column chromatography (SiO₂, DCM:MeOH=10:1, R_{f}=0.40) to give the title compound (450 mg, 666 μmol, 40.9% yield, 69.7% purity in LCMS at 220 nm) as yellow solid. (ESI⁺) m/z: 387.1 (M-84)⁺, (C₂₇H₂₆N₄O₄).

D. 3-(1-Oxo-5-(5-phenyl-1H-pyrazol-3-yl)isoindolin-2-yl)piperidine-2,6-dione: To a solution of 3-(1-oxo-5-(5-phenyl-1-(tetrahydro-2H-pyran-2-yl)-1H-pyrazol-3-yl)isoindolin-2-yl)piperidine-2,6-dione (450 mg, 956 μmol, 1.00 eq) in DCM (6.00 mL) was added HCl/dioxane (4.0 M, 239 μL, 1.00 eq). The mixture was stirred at 20° C. for 6 h. The mixture was concentrated under reduced pressure to get residue. The crude product was purified by preparative-HPLC (using a Phenomenex luna C18 (150 mm×25 mm 10 μm) and gradient of 20-50% acetonitrile in water containing 0.5% TFA over 15 min at a flow rate of 25 mL/min) to give the title compound (357 mg, 906 μmol, 94.7% yield, 98.1% purity in HPLC at 220 nm) as an off-white solid. ¹H NMR: (400 MHz, DMSO-d₆) δ 11.0 (s, 1H), 8.07-8.00 (m, 1H), 7.85-7.83 (m, 1H), 7.80-7.78 (m, 3H), 7.49-7.45 (m, 2H), 7.37-7.33 (m, 2H), 5.18-5.11 (m, 1H), 4.58-4.37 (m, 2H), 2.96-2.89 (m, 1H), 2.63-2.59 (m, 1H), 2.45-2.40 (m, 1H), 2.04-2.02 (m, 1H). (ESI⁺) m/z: 386.9 (M+H)⁺, (C₂₂H₁₈N₄O₃).

### Example 211

### Synthesis of 3-(5-(1,4-Dimethyl-5-phenyl-1H-pyrazol-3-yl)-1-oxoisoindolin-2-yl)piperidine-2,6-dione

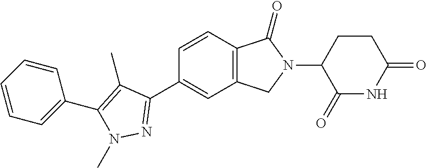 [SMILES: Cc1c(-c2ccc3c(c2)CN(C2CCC(=O)NC2=O)C3=O)nn(C)c1-c1ccccc1]

A. 5-Bromo-1,4-dimethyl-1H-pyrazole: To a solution of 1,4-dimethylpyrazole (5.00 g, 52.0 mmol, 1.00 eq) in CHCl₃ (150 mL) was added NBS (11.1 g, 62.4 mmol, 1.20 eq). The mixture was stirred at 25° C. for 12 h. Then the reaction mixture was filtered and the filtrate was concentrated in vacuum to get a residue. The residue was purified by column chromatography (SiO₂, Petroleum ether:Ethyl acetate=100:1 to 5:1, R_{f}=0.60 (Petroleum ether:Ethyl acetate=5:1)) to give the title compound (2.00 g, 11.4 mmol, 21.9% yield, 100% purity in LCMS at 220 nm) as yellow oil. ¹H NMR: (400 MHz, CDCl₃) δ 7.33 (s, 1H), 3.84 (s, 3H), 2.00 (s, 3H). (ESI⁺) m/z: 175.0 (M+H)⁺, (C₅H₇BrN₂).

B. 1,4-Dimethyl-5-phenyl-1H-pyrazole: To a solution of 5-bromo-1,4-dimethylpyrazole (1.00 g, 5.71 mmol, 1.00 eq) in dioxane (20.0 mL) and H₂O (2.00 mL) was added phenylboronic acid (835 mg, 6.86 mmol, 1.20 eq), K₃PO₄ (3.64 g, 17.1 mmol, 3.00 eq) and Pd(dppf)Cl₂ (418 mg, 571 μmol, 0.10 eq) under N₂. The mixture was stirred at 70° C. for 2 h under N₂. After the reaction was completed, the reaction mixture was filtered and the filtrate was concentrated in vacuum to give a residue. The residue was purified by column chromatography (SiO₂, Petroleum ether:Ethyl acetate=100:1 to 3:1, R_{f}=0.40 (Petroleum ether:Ethyl acetate=3:1)) to give the title compound (850 mg, 4.61 mmol, 80.7% yield, 93.5% purity in LCMS at 220 nm) as yellow oil. (ESI⁺) m/z: 173.1 (M+H)⁺, (C₁₁H₁₂N₂).

C. 3-Bromo-1,4-dimethyl-5-phenyl-1H-pyrazole: To a solution of 1,4-dimethyl-5-phenyl-pyrazole (850 mg, 4.61 mmol, 1.00 eq) in ACN (10.0 mL) was added NBS (862 mg, 4.85 mmol, 1.05 eq) at 0° C. under N₂. The reaction mixture was stirred at 25° C. for 2 h under N₂. After the reaction was completed, the reaction mixture was poured into H₂O (30.0 mL) and extracted with ethyl acetate (3×30.0 mL). The combined organic layer was washed with brine (3×30.0 mL), dried over Na₂SO₄, filtered and the filtrate was concentrated in vacuum to give a residue. The residue was purified by column chromatography (SiO₂, Petroleum ether:Ethyl acetate=10:1 to 3:1, R_{f}=0.60 (Petroleum ether:Ethyl acetate=3:1)) to give the title compound (940 mg, 3.74 mmol, 77.5% yield, 100% purity in LCMS at 220 nm) as a yellow solid. ¹H NMR: (400 MHz, CDCl₃) δ 7.50-7.45 (m, 3H), 7.32-7.29 (m, 2H), 3.74 (s, 3H), 1.96 (s, 3H). (ESI⁺) m/z: 251.0 (M+H)⁺, (C₁₁H₁₁BrN₂).

D. 3-(5-(1,4-Dimethyl-5-phenyl-1H-pyrazol-3-yl)-1-oxoisoindolin-2-yl)piperidine-2,6-dione: To a solution of 3-bromo-1,4-dimethyl-5-phenyl-pyrazole (200 mg, 796 μmol, 1.00 eq) in dioxane (8.00 mL) and H₂O (0.40 mL) was added 3-[1-oxo-5-(4,4,5,5-tetramethyl-1,3,2-dioxaborolan-2-yl)isoindolin-2-yl]piperidine-2,6-dione (737 mg, 1.99 mmol, 2.50 eq), K₃PO₄ (338 mg, 1.59 mmol, 2.00 eq) and Ru-Phos-Pd-G₃ (66.6 mg, 79.6 μmol, 0.10 eq). The mixture was stirred at 100° C. for 2 h under N₂. After the reaction was completed, the reaction mixture was filtered and the filtrate was concentrated in vacuum to give a residue. The residue was purified by preparative-HPLC (using a Phenomenex Luna C18 (150 mm×25 mm×10 μm) and gradient of 25-55% acetonitrile in water containing 0.1% TFA over 10 min at a flow rate of 25.0 mL/min) to give the title compound (77.7 mg, 186 μmol, 23.3% yield, 99.2% purity in HPLC at 220 nm) as a white solid. ¹H NMR: (400 MHz, DMSO-d₆) δ 11.0 (s, 1H), 7.92 (s, 1H), 7.85 (d, J=7.2 Hz, 1H), 7.79 (d, J=7.6 Hz, 1H), 7.57 (t, J=7.6 Hz, 2H), 7.53-7.47 (m, 3H), 5.14 (dd, J=13.2, 4.8 Hz, 1H), 4.46 (dd, J=52.8, 17.2 Hz, 2H), 3.78 (s, 3H), 2.97-2.89 (m, 1H), 2.68-2.66 (m, 1H), 2.33-2.32 (m, 1H), 2.15 (s, 3H), 2.06-2.00 (m, 1H). (ESI⁺) m/z: 415.1 (M+H)⁺, (C₂₄H₂₂N₄O₃).

### Example 212

### Synthesis of 3-(1-Oxo-5-(8H-pyrazolo[5,1-a]isoindol-3-yl)isoindolin-2-yl)piperidine-2,6-dione

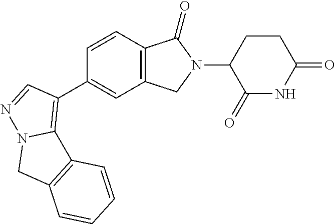 [SMILES: O=C1CCC(N2Cc3cc(-c4cnn5c4-c4ccccc4C5)ccc3C2=O)C(=O)N1]

A. 1-(2-Iodobenzyl)-1H-pyrazole: To a solution of 1H-pyrazole (1.38 g, 20.2 mmol, 1.20 eq) in THF (25.0 mL) was added NaH (1.01 g, 25.2 mmol, 60.0% purity, 1.50 eq) portion-wise at 0° C. The mixture was stirred at 20° C. for 1 h and was added 1-(bromomethyl)-2-iodobenzene (5.00 g, 16.8 mmol, 1.00 eq). Then the mixture was stirred at 20° C. for 12 h. The reaction mixture was poured into H₂O (100 mL) and extracted with Ethyl acetate (3×25.0 mL). The combined organic layer was washed with brine (3×25.0 mL), and dried over by Na₂SO₄, filtered and concentrate filtrate under reduced pressure to give the title compound (4.00 g, 13.7 mmol, 81.6% yield, 97.6% purity in LCMS at 220 nm) as light yellow oil. ¹H NMR: (400 MHz, CDCl₃) (8.31-8.29 (m, 1H), 7.98-7.94 (m, 1H), 7.87-7.85 (m, 1H), 7.47-7.30 (m, 1H), 7.27-7.02 (m, 1H), 6.98-6.85 (m, 1H), 6.32 (s, 1H), 5.93 (s, 2H). (ESI⁺) m/z: 284.9 (M+H)⁺, (C₁₀H₉IN₂).

B. 8H-Pyrazolo[5,1-a]isoindole: To a solution of 1-(2-iodobenzyl)-1H-pyrazole (3.00 g, 10.5 mmol, 1.00 eq), K₂CO₃ (2.92 g, 21.1 mmol, 2.00 eq), LiCl (1.34 g, 31.6 mmol, 649 μL, 3.00 eq) and PivOH (323 mg, 3.17 mmol, 363 μL, 0.30 eq) in DMA (20.0 mL) was added Pd(OAc)₂ (355 mg, 1.58 mmol, 0.15 eq) under N₂. The mixture was stirred at 145° C. for 14 h under N₂. The reaction mixture was poured into H₂O (50.0 mL) and extracted with Ethyl acetate (3×50.0 mL). The combined organic layer was washed with brine (3×50.0 mL), and dried over by Na₂SO₄, filtered and concentrate filtrate under reduced pressure to get residue. The residue was purified by column chromatography (SiO₂, Petroleum ether:Ethyl acetate=3:1, TLC:Petroleum ether:Ethyl acetate=3:1, R_{f}=0.40) to give the title compound (330 mg, 1.81 mmol, 17.1% yield, 94.0% purity in LCMS at 220 nm) as light yellow oil. ¹H NMR: (400 MHz, CDCl₃) (7.67-7.66 (m, 1H), 7.61-7.59 (m, 1H), 7.49-7.42 (m, 1H), 7.36-7.35 (m, 1H), 7.34-7.27 (m, 1H), 6.48-6.36 (m, 1H) 5.18-5.11 (m, 2H). (ESI⁺) m/z: 157.0 (M+H)⁺, (C₁₀H₈N₂).

C. 3-Bromo-8H-pyrazolo[5,1-a]isoindole: To a solution of 8H-pyrazolo[5,1-a]isoindole (150 mg, 902 μmol, 1.00 eq) in ACN (6.00 mL) was added NBS (176 mg, 993 μmol, 1.10 eq) at 0° C. The mixture was stirred at 20° C. for 1 h. The reaction mixture was poured into H₂O (30.0 mL) and extracted with Ethyl acetate (3×30.0 mL). The combined organic layer was washed with brine (3×30.0 mL), and dried over by Na₂SO₄, filtered and concentrate filtrate under reduced pressure to give the title compound (200 mg, 816 μmol, 90.4% yield, 96.0% purity in LCMS at 220 nm) as light yellow solid. ¹H NMR: (400 MHz, CDCl₃) δ 7.86-7.83 (m, 1H), 7.63-7.49 (m, 1H), 7.49-7.38 (m, 3H), 5.24 (s, 2H) (ESI⁺) m/z: 234.8 (M+H)⁺, (C₁₀H₇BrN₂).

D. 3-(1-Oxo-5-(8H-pyrazolo[5,1-a]isoindol-3-yl)isoindolin-2-yl)piperidine-2,6-dione: To a solution of 3-bromo-8H-pyrazolo[5,1-a]isoindole (150 mg, 627 μmol, 1.00 eq), 3-(1-oxo-5-(4,4,5,5-tetramethyl-1,3,2-dioxaborolan-2-yl)isoindolin-2-yl)piperidine-2,6-dione (812 mg, 2.20 mmol, 3.50 eq) and K₃PO₄ (399 mg, 1.88 mmol, 3.00 eq) in dioxane (5.00 mL) and H₂O (0.25 mL) was added Ru-Phos-Pd-G₃ (52.4 mg, 62.7 μmol, 0.10 eq) under N₂. The mixture was stirred at 100° C. for 2 h under N₂. The reaction mixture was concentrated under reduced pressure to get a residue at 45° C. The residue was purified by preparative-HPLC (using a Phenomenex luna C18 (150×25 mm×5 μm) and gradient of 18.00%-48.0% acetonitrile in water containing 0.5% TFA over 10 min at a flow rate of 25 mL/min) to give the title compound (115 mg, 284 μmol, 45.8% yield, 99.0% purity in HPLC at 220 nm) as white solid. ¹H NMR: (400 MHz, DMSO-d₆) δ 11.1-11.0 (m, 1H), 7.98-7.90 (m, 1H), 7.88-7.79 (m, 4H), 7.65-7.47 (m, 1H), 7.46-7.44 (m, 2H), 5.32-5.17 (m, 2H), 5.16-5.13 (m, 1H), 4.58-4.41 (m, 2H), 2.98-2.93 (m, 1H), 2.64-2.60 (m, 1H), 2.41-2.40 (m, 1H), 2.07-2.02 (m, 1H). (ESI⁺) m/z: 399.0 (M+H)⁺, (C₂₃H₁₈N₄O₃).

### Examples 213-225

The compounds of Examples 213-225 were prepared according to the procedure of Scheme 1 as shown in Examples 55-116.

| | | MW + 1 | Step 1 |
|---|---|---|---|
| Example | Structure | (Observed) | Conditions |
| 213 | 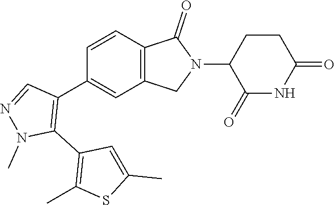 [SMILES: Cc1cc(-c2c(-c3ccc4c(c3)CN(C3CCC(=O)NC3=O)C4=O)cnn2C)c(C)s1] | 435.2 | 1 |
| 214 | 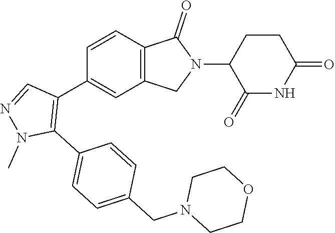 [SMILES: Cn1ncc(-c2ccc3c(c2)CN(C2CCC(=O)NC2=O)C3=O)c1-c1ccc(CN2CCOCC2)cc1] | 500.3 | 1 |
| 215 | 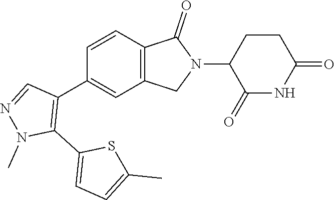 [SMILES: Cc1ccc(-c2c(-c3ccc4c(c3)CN(C3CCC(=O)NC3=O)C4=O)cnn2C)s1] | 421.2 | 1 |
| 216 | 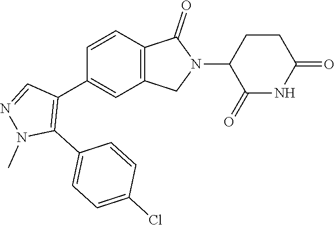 [SMILES: Cn1ncc(-c2ccc3c(c2)CN(C2CCC(=O)NC2=O)C3=O)c1-c1ccc(Cl)cc1] | 435.1 | 1 |
| 217 | 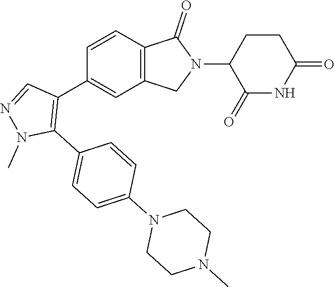 [SMILES: CN1CCN(c2ccc(-c3c(-c4ccc5c(c4)CN(C4CCC(=O)NC4=O)C5=O)cnn3C)cc2)CC1] | 499.3 | 1 |
| 218 | 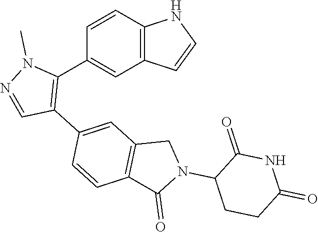 [SMILES: Cn1ncc(-c2ccc3c(c2)CN(C2CCC(=O)NC2=O)C3=O)c1-c1ccc2[nH]ccc2c1] | 440.3 | 1 |
| 219 | 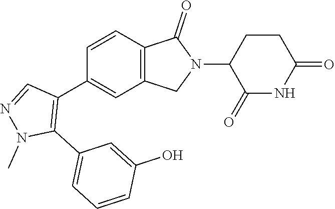 [SMILES: Cn1ncc(-c2ccc3c(c2)CN(C2CCC(=O)NC2=O)C3=O)c1-c1cccc(O)c1] | 417.3 | 1 |
| 220 | 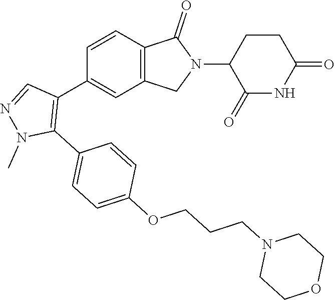 [SMILES: Cn1ncc(-c2ccc3c(c2)CN(C2CCC(=O)NC2=O)C3=O)c1-c1ccc(OCCCN2CCOCC2)cc1] | 544.4 | 1 |
| 221 | 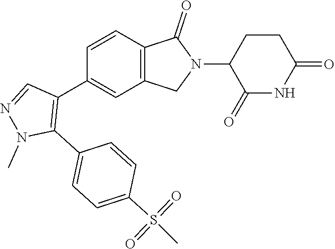 [SMILES: Cn1ncc(-c2ccc3c(c2)CN(C2CCC(=O)NC2=O)C3=O)c1-c1ccc(S(C)(=O)=O)cc1] | 479.2 | 1 |
| 222 | 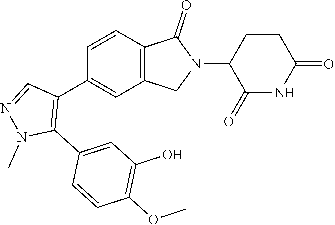 [SMILES: COc1ccc(-c2c(-c3ccc4c(c3)CN(C3CCC(=O)NC3=O)C4=O)cnn2C)cc1O] | 447.2 | 1 |
| 223 | 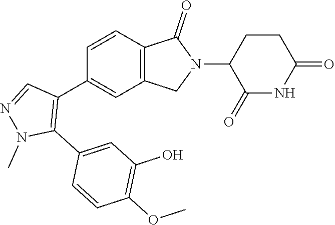 [SMILES: COc1ccc(-c2c(-c3ccc4c(c3)CN(C3CCC(=O)NC3=O)C4=O)cnn2C)cc1O] | 513.3 | 1 |
| 224 | 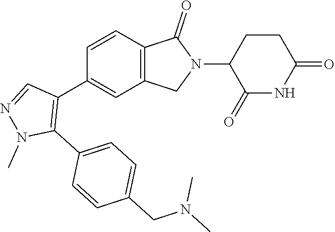 [SMILES: CN(C)Cc1ccc(-c2c(-c3ccc4c(c3)CN(C3CCC(=O)NC3=O)C4=O)cnn2C)cc1] | 458.3 | 1 |
| 225 | 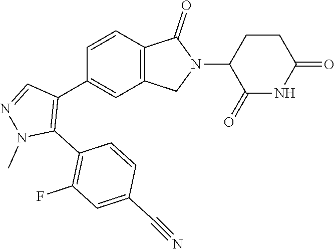 [SMILES: Cn1ncc(-c2ccc3c(c2)CN(C2CCC(=O)NC2=O)C3=O)c1-c1ccc(C#N)cc1F] | 444.2 | 1 |

### Examples 226-316

The compounds of Examples 226-316 were prepared according to the procedure of Scheme 2 as shown in Examples 161-204.

| | | MW + 1 |
|---|---|---|
| Example | Structure | (Observed) |
| 226 | 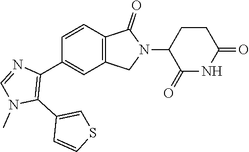 [SMILES: Cn1cnc(-c2ccc3c(c2)CN(C2CCC(=O)NC2=O)C3=O)c1-c1ccsc1] | 407.2 |
| 227 | 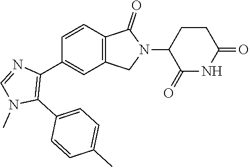 [SMILES: Cc1ccc(-c2c(-c3ccc4c(c3)CN(C3CCC(=O)NC3=O)C4=O)ncn2C)cc1] | 415.3 |
| 228 | 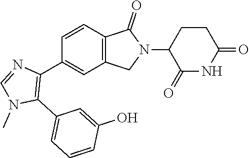 [SMILES: Cn1cnc(-c2ccc3c(c2)CN(C2CCC(=O)NC2=O)C3=O)c1-c1cccc(O)c1] | 417.2 |
| 229 | 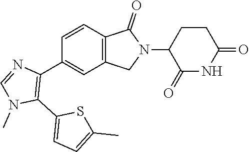 [SMILES: Cc1ccc(-c2c(-c3ccc4c(c3)CN(C3CCC(=O)NC3=O)C4=O)ncn2C)s1] | 421.2 |
| 230 | 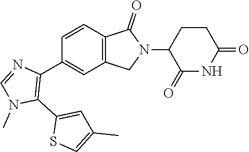 [SMILES: Cc1csc(-c2c(-c3ccc4c(c3)CN(C3CCC(=O)NC3=O)C4=O)ncn2C)c1] | 421.2 |
| 231 | 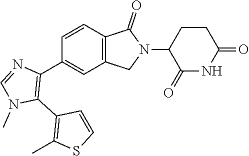 [SMILES: Cc1sccc1-c1c(-c2ccc3c(c2)CN(C2CCC(=O)NC2=O)C3=O)ncn1C] | 421.2 |
| 232 | 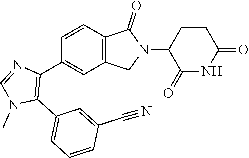 [SMILES: Cn1cnc(-c2ccc3c(c2)CN(C2CCC(=O)NC2=O)C3=O)c1-c1cccc(C#N)c1] | 426.3 |
| 233 | 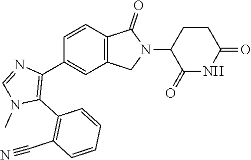 [SMILES: Cn1cnc(-c2ccc3c(c2)CN(C2CCC(=O)NC2=O)C3=O)c1-c1ccccc1C#N] | 426.2 |
| 234 | 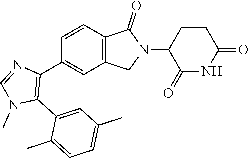 [SMILES: Cc1ccc(C)c(-c2c(-c3ccc4c(c3)CN(C3CCC(=O)NC3=O)C4=O)ncn2C)c1] | 429.3 |
| 235 | 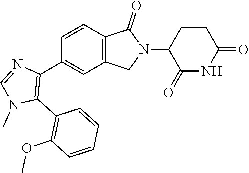 [SMILES: COc1ccccc1-c1c(-c2ccc3c(c2)CN(C2CCC(=O)NC2=O)C3=O)ncn1C] | 431.3 |
| 236 | 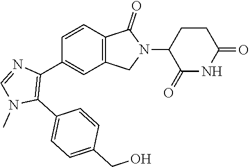 [SMILES: Cn1cnc(-c2ccc3c(c2)CN(C2CCC(=O)NC2=O)C3=O)c1-c1ccc(CO)cc1] | 431.2 |
| 237 | 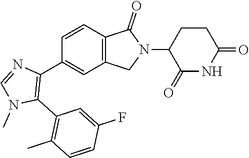 [SMILES: Cc1ccc(F)cc1-c1c(-c2ccc3c(c2)CN(C2CCC(=O)NC2=O)C3=O)ncn1C] | 433.2 |
| 238 | 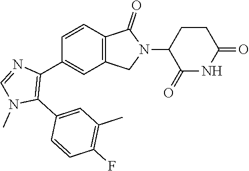 [SMILES: Cc1cc(-c2c(-c3ccc4c(c3)CN(C3CCC(=O)NC3=O)C4=O)ncn2C)ccc1F] | 433.3 |
| 239 | 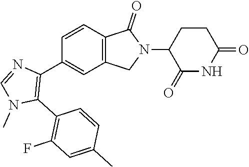 [SMILES: Cc1ccc(-c2c(-c3ccc4c(c3)CN(C3CCC(=O)NC3=O)C4=O)ncn2C)c(F)c1] | 433.2 |
| 240 | 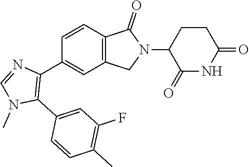 [SMILES: Cc1ccc(-c2c(-c3ccc4c(c3)CN(C3CCC(=O)NC3=O)C4=O)ncn2C)cc1F] | 433.3 |
| 241 | 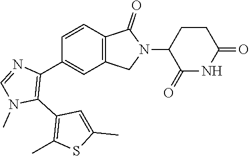 [SMILES: Cc1cc(-c2c(-c3ccc4c(c3)CN(C3CCC(=O)NC3=O)C4=O)ncn2C)c(C)s1] | 435.2 |
| 242 | 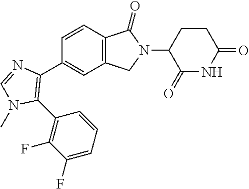 [SMILES: Cn1cnc(-c2ccc3c(c2)CN(C2CCC(=O)NC2=O)C3=O)c1-c1cccc(F)c1F] | 437.2 |
| 243 | 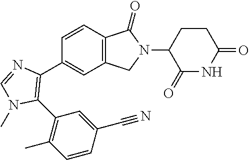 [SMILES: Cc1ccc(C#N)cc1-c1c(-c2ccc3c(c2)CN(C2CCC(=O)NC2=O)C3=O)ncn1C] | 440.3 |
| 244 | 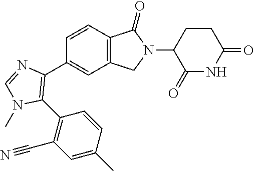 [SMILES: Cc1ccc(-c2c(-c3ccc4c(c3)CN(C3CCC(=O)NC3=O)C4=O)ncn2C)c(C#N)c1] | 440.3 |
| 245 | 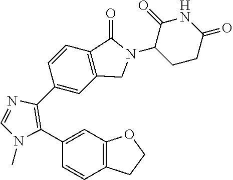 [SMILES: Cn1cnc(-c2ccc3c(c2)CN(C2CCC(=O)NC2=O)C3=O)c1-c1ccc2c(c1)OCC2] | 443.2 |
| 246 | 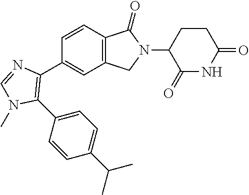 [SMILES: CC(C)c1ccc(-c2c(-c3ccc4c(c3)CN(C3CCC(=O)NC3=O)C4=O)ncn2C)cc1] | 443.3 |
| 247 | 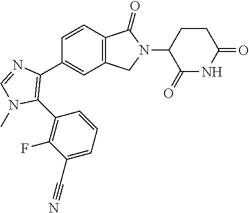 [SMILES: Cn1cnc(-c2ccc3c(c2)CN(C2CCC(=O)NC2=O)C3=O)c1-c1cccc(C#N)c1F] | 444.2 |
| 248 | 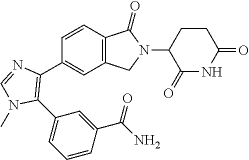 [SMILES: Cn1cnc(-c2ccc3c(c2)CN(C2CCC(=O)NC2=O)C3=O)c1-c1cccc(C(N)=O)c1] | 444.3 |
| 249 | 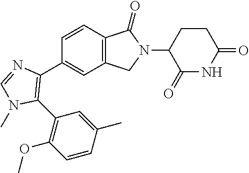 [SMILES: COc1ccc(C)cc1-c1c(-c2ccc3c(c2)CN(C2CCC(=O)NC2=O)C3=O)ncn1C] | 445.3 |
| 250 | 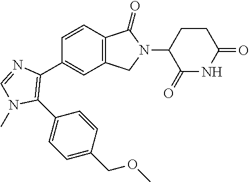 [SMILES: COCc1ccc(-c2c(-c3ccc4c(c3)CN(C3CCC(=O)NC3=O)C4=O)ncn2C)cc1] | 445.3 |
| 251 | 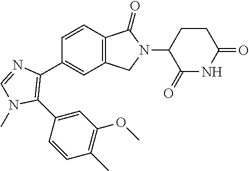 [SMILES: COc1cc(-c2c(-c3ccc4c(c3)CN(C3CCC(=O)NC3=O)C4=O)ncn2C)ccc1C] | 445.3 |
| 252 | 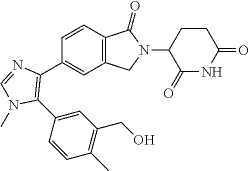 [SMILES: Cc1ccc(-c2c(-c3ccc4c(c3)CN(C3CCC(=O)NC3=O)C4=O)ncn2C)cc1CO] | 445.3 |
| 253 | 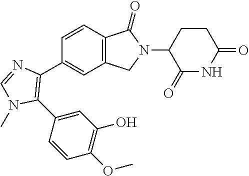 [SMILES: COc1ccc(-c2c(-c3ccc4c(c3)CN(C3CCC(=O)NC3=O)C4=O)ncn2C)cc1O] | 447.3 |
| 254 | 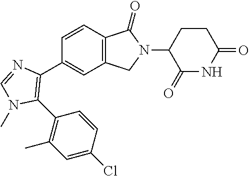 [SMILES: Cc1cc(Cl)ccc1-c1c(-c2ccc3c(c2)CN(C2CCC(=O)NC2=O)C3=O)ncn1C] | 449.2 |
| 255 | 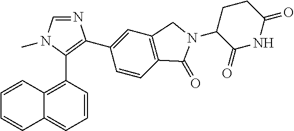 [SMILES: Cn1cnc(-c2ccc3c(c2)CN(C2CCC(=O)NC2=O)C3=O)c1-c1cccc2ccccc12] | 451.3 |
| 256 | 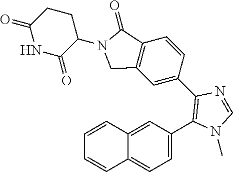 [SMILES: Cn1cnc(-c2ccc3c(c2)CN(C2CCC(=O)NC2=O)C3=O)c1-c1ccc2ccccc2c1] | 451.3 |
| 257 | 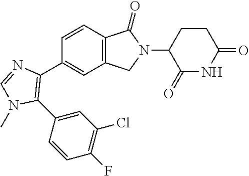 [SMILES: Cn1cnc(-c2ccc3c(c2)CN(C2CCC(=O)NC2=O)C3=O)c1-c1ccc(F)c(Cl)c1] | 453.2 |
| 258 | 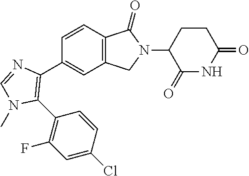 [SMILES: Cn1cnc(-c2ccc3c(c2)CN(C2CCC(=O)NC2=O)C3=O)c1-c1ccc(Cl)cc1F] | 453.2 |
| 259 | 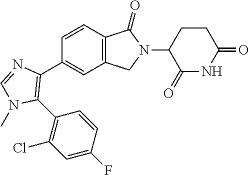 [SMILES: Cn1cnc(-c2ccc3c(c2)CN(C2CCC(=O)NC2=O)C3=O)c1-c1ccc(F)cc1Cl] | 453.2 |
| 260 | 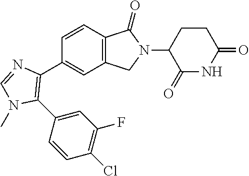 [SMILES: Cn1cnc(-c2ccc3c(c2)CN(C2CCC(=O)NC2=O)C3=O)c1-c1ccc(Cl)c(F)c1] | 453.2 |
| 261 | 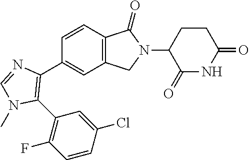 [SMILES: Cn1cnc(-c2ccc3c(c2)CN(C2CCC(=O)NC2=O)C3=O)c1-c1cc(Cl)ccc1F] | 453.2 |
| 262 | 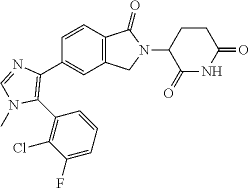 [SMILES: Cn1cnc(-c2ccc3c(c2)CN(C2CCC(=O)NC2=O)C3=O)c1-c1cccc(F)c1Cl] | 453.2 |
| 263 | 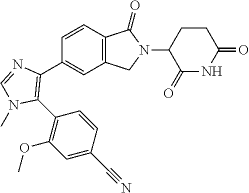 [SMILES: COc1cc(C#N)ccc1-c1c(-c2ccc3c(c2)CN(C2CCC(=O)NC2=O)C3=O)ncn1C] | 456.2 |
| 264 | 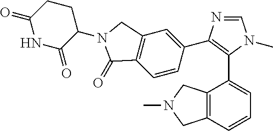 [SMILES: CN1Cc2cccc(-c3c(-c4ccc5c(c4)CN(C4CCC(=O)NC4=O)C5=O)ncn3C)c2C1] | 456.3 |
| 265 | 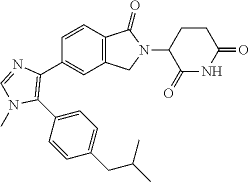 [SMILES: CC(C)Cc1ccc(-c2c(-c3ccc4c(c3)CN(C3CCC(=O)NC3=O)C4=O)ncn2C)cc1] | 457.3 |
| 266 | 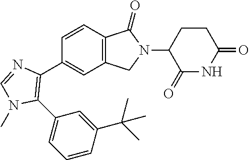 [SMILES: Cn1cnc(-c2ccc3c(c2)CN(C2CCC(=O)NC2=O)C3=O)c1-c1cccc(C(C)(C)C)c1] | 457.3 |
| 267 | 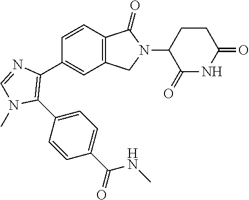 [SMILES: CNC(=O)c1ccc(-c2c(-c3ccc4c(c3)CN(C3CCC(=O)NC3=O)C4=O)ncn2C)cc1] | 458.3 |
| 268 | 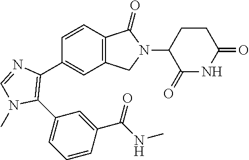 [SMILES: CNC(=O)c1cccc(-c2c(-c3ccc4c(c3)CN(C3CCC(=O)NC3=O)C4=O)ncn2C)c1] | 458.3 |
| 269 | 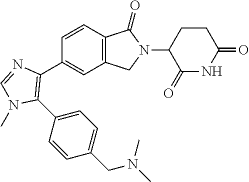 [SMILES: CN(C)Cc1ccc(-c2c(-c3ccc4c(c3)CN(C3CCC(=O)NC3=O)C4=O)ncn2C)cc1] | 458.3 |
| 270 | 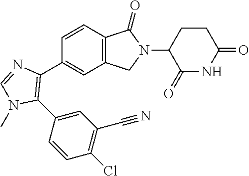 [SMILES: Cn1cnc(-c2ccc3c(c2)CN(C2CCC(=O)NC2=O)C3=O)c1-c1ccc(Cl)c(C#N)c1] | 460.2 |
| 271 | 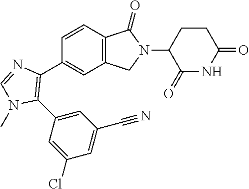 [SMILES: Cn1cnc(-c2ccc3c(c2)CN(C2CCC(=O)NC2=O)C3=O)c1-c1cc(Cl)cc(C#N)c1] | 460.2 |
| 272 | 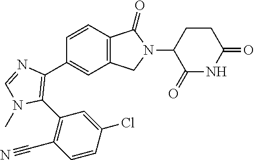 [SMILES: Cn1cnc(-c2ccc3c(c2)CN(C2CCC(=O)NC2=O)C3=O)c1-c1cc(Cl)ccc1C#N] | 460.2 |
| 273 | 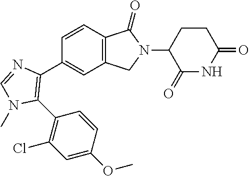 [SMILES: COc1ccc(-c2c(-c3ccc4c(c3)CN(C3CCC(=O)NC3=O)C4=O)ncn2C)c(Cl)c1] | 465.2 |
| 274 | 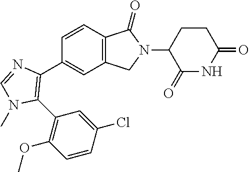 [SMILES: COc1ccc(Cl)cc1-c1c(-c2ccc3c(c2)CN(C2CCC(=O)NC2=O)C3=O)ncn1C] | 465.2 |
| 275 | 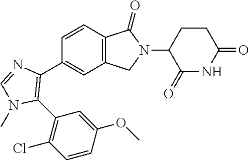 [SMILES: COc1ccc(Cl)c(-c2c(-c3ccc4c(c3)CN(C3CCC(=O)NC3=O)C4=O)ncn2C)c1] | 465.2 |
| 276 | 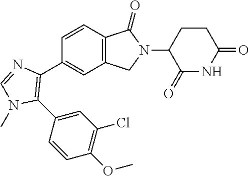 [SMILES: COc1ccc(-c2c(-c3ccc4c(c3)CN(C3CCC(=O)NC3=O)C4=O)ncn2C)cc1Cl] | 465.3 |
| 277 | 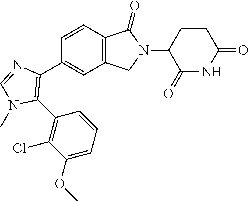 [SMILES: COc1cccc(-c2c(-c3ccc4c(c3)CN(C3CCC(=O)NC3=O)C4=O)ncn2C)c1Cl] | 465.2 |
| 278 | 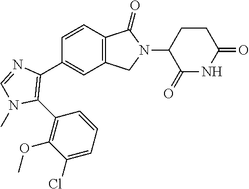 [SMILES: COc1c(Cl)cccc1-c1c(-c2ccc3c(c2)CN(C2CCC(=O)NC2=O)C3=O)ncn1C] | 465.2 |
| 279 | 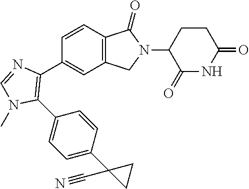 [SMILES: Cn1cnc(-c2ccc3c(c2)CN(C2CCC(=O)NC2=O)C3=O)c1-c1ccc(C2(C#N)CC2)cc1] | 466.3 |
| 280 | 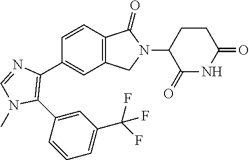 [SMILES: Cn1cnc(-c2ccc3c(c2)CN(C2CCC(=O)NC2=O)C3=O)c1-c1cccc(C(F)(F)F)c1] | 469.2 |
| 281 | 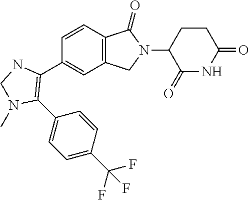 [SMILES: CN1CNC(c2ccc3c(c2)CN(C2CCC(=O)NC2=O)C3=O)=C1c1ccc(C(F)(F)F)cc1] | 469.2 |
| 282 | 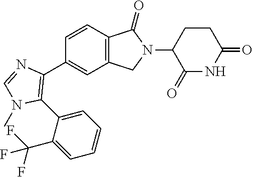 [SMILES: Cn1cnc(-c2ccc3c(c2)CN(C2CCC(=O)NC2=O)C3=O)c1-c1ccccc1C(F)(F)F] | 469.2 |
| 283 | 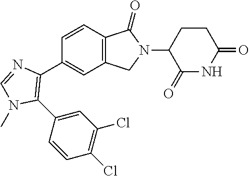 [SMILES: Cn1cnc(-c2ccc3c(c2)CN(C2CCC(=O)NC2=O)C3=O)c1-c1ccc(Cl)c(Cl)c1] | 469.2 |
| 284 | 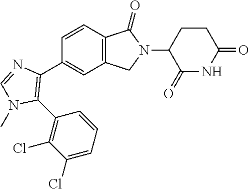 [SMILES: Cn1cnc(-c2ccc3c(c2)CN(C2CCC(=O)NC2=O)C3=O)c1-c1cccc(Cl)c1Cl] | 469.2 |
| 285 | 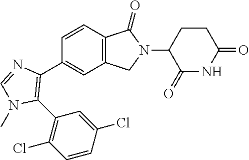 [SMILES: Cn1cnc(-c2ccc3c(c2)CN(C2CCC(=O)NC2=O)C3=O)c1-c1cc(Cl)ccc1Cl] | 469.2 |
| 286 | 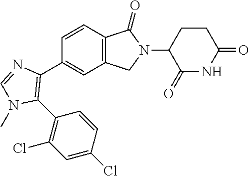 [SMILES: Cn1cnc(-c2ccc3c(c2)CN(C2CCC(=O)NC2=O)C3=O)c1-c1ccc(Cl)cc1Cl] | 469.2 |
| 287 | 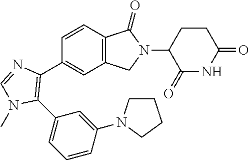 [SMILES: Cn1cnc(-c2ccc3c(c2)CN(C2CCC(=O)NC2=O)C3=O)c1-c1cccc(N2CCCC2)c1] | 470.3 |
| 288 | 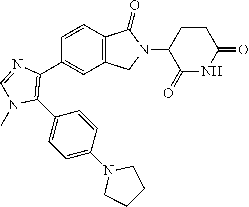 [SMILES: Cn1cnc(-c2ccc3c(c2)CN(C2CCC(=O)NC2=O)C3=O)c1-c1ccc(N2CCCC2)cc1] | 470.3 |
| 289 | 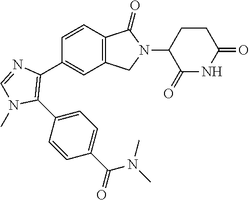 [SMILES: CN(C)C(=O)c1ccc(-c2c(-c3ccc4c(c3)CN(C3CCC(=O)NC3=O)C4=O)ncn2C)cc1] | 472.3 |
| 290 | 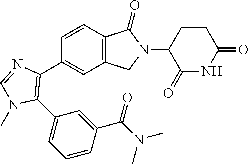 [SMILES: CN(C)C(=O)c1cccc(-c2c(-c3ccc4c(c3)CN(C3CCC(=O)NC3=O)C4=O)ncn2C)c1] | 472.3 |
| 291 | 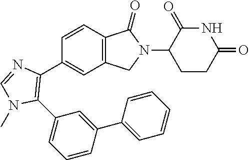 [SMILES: Cn1cnc(-c2ccc3c(c2)CN(C2CCC(=O)NC2=O)C3=O)c1-c1cccc(-c2ccccc2)c1] | 477.3 |
| 292 | 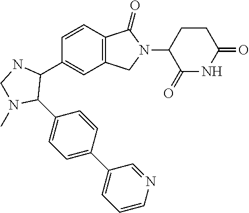 [SMILES: CN1CNC(c2ccc3c(c2)CN(C2CCC(=O)NC2=O)C3=O)C1c1ccc(-c2cccnc2)cc1] | 478.3 |
| 293 | 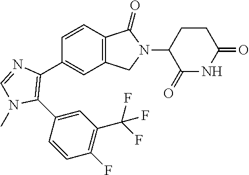 [SMILES: Cn1cnc(-c2ccc3c(c2)CN(C2CCC(=O)NC2=O)C3=O)c1-c1ccc(F)c(C(F)(F)F)c1] | 487.2 |
| 294 | 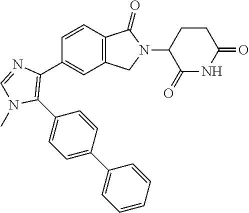 [SMILES: Cn1cnc(-c2ccc3c(c2)CN(C2CCC(=O)NC2=O)C3=O)c1-c1ccc(-c2ccccc2)cc1] | 477.3 |
| 295 | 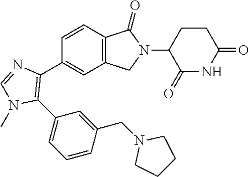 [SMILES: Cn1cnc(-c2ccc3c(c2)CN(C2CCC(=O)NC2=O)C3=O)c1-c1cccc(CN2CCCC2)c1] | 484.3 |
| 296 | 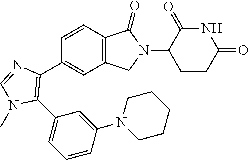 [SMILES: Cn1cnc(-c2ccc3c(c2)CN(C2CCC(=O)NC2=O)C3=O)c1-c1cccc(N2CCCCC2)c1] | 484.3 |
| 297 | 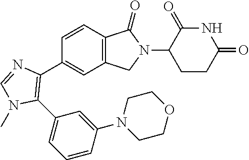 [SMILES: Cn1cnc(-c2ccc3c(c2)CN(C2CCC(=O)NC2=O)C3=O)c1-c1cccc(N2CCOCC2)c1] | 486.3 |
| 298 | 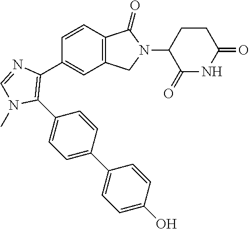 [SMILES: Cn1cnc(-c2ccc3c(c2)CN(C2CCC(=O)NC2=O)C3=O)c1-c1ccc(-c2ccc(O)cc2)cc1] | 493.3 |
| 299 | 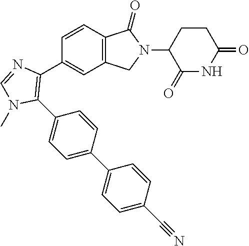 [SMILES: Cn1cnc(-c2ccc3c(c2)CN(C2CCC(=O)NC2=O)C3=O)c1-c1ccc(-c2ccc(C#N)cc2)cc1] | 502.3 |
| 300 | 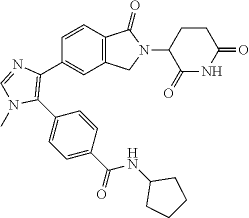 [SMILES: Cn1cnc(-c2ccc3c(c2)CN(C2CCC(=O)NC2=O)C3=O)c1-c1ccc(C(=O)NC2CCCC2)cc1] | 512.3 |
| 301 | 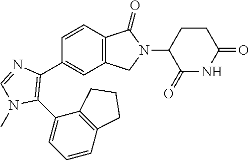 [SMILES: Cn1cnc(-c2ccc3c(c2)CN(C2CCC(=O)NC2=O)C3=O)c1-c1cccc2c1CCC2] | 441.3 |
| 302 | 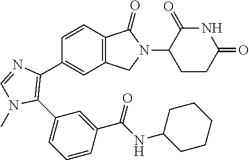 [SMILES: Cn1cnc(-c2ccc3c(c2)CN(C2CCC(=O)NC2=O)C3=O)c1-c1cccc(C(=O)NC2CCCCC2)c1] | 526.3 |
| 303 | 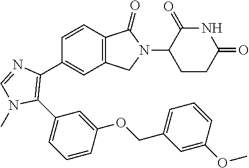 [SMILES: COc1cccc(COc2cccc(-c3c(-c4ccc5c(c4)CN(C4CCC(=O)NC4=O)C5=O)ncn3C)c2)c1] | 537.3 |
| 304 | 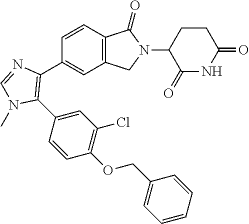 [SMILES: Cn1cnc(-c2ccc3c(c2)CN(C2CCC(=O)NC2=O)C3=O)c1-c1ccc(OCc2ccccc2)c(Cl)c1] | 541.3 |
| 305 | 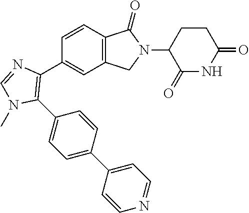 [SMILES: Cn1cnc(-c2ccc3c(c2)CN(C2CCC(=O)NC2=O)C3=O)c1-c1ccc(-c2ccncc2)cc1] | 478.3 |
| 306 | 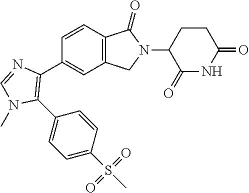 [SMILES: Cn1cnc(-c2ccc3c(c2)CN(C2CCC(=O)NC2=O)C3=O)c1-c1ccc(S(C)(=O)=O)cc1] | 479.2 |
| 307 | 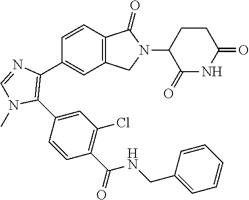 [SMILES: Cn1cnc(-c2ccc3c(c2)CN(C2CCC(=O)NC2=O)C3=O)c1-c1ccc(C(=O)NCc2ccccc2)c(Cl)c1] | 568.3 |
| 308 | 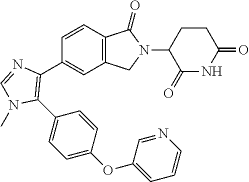 [SMILES: Cn1cnc(-c2ccc3c(c2)CN(C2CCC(=O)NC2=O)C3=O)c1-c1ccc(Oc2cccnc2)cc1] | 494.3 |
| 309 | 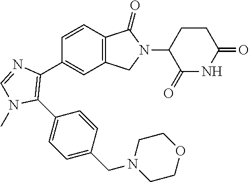 [SMILES: Cn1cnc(-c2ccc3c(c2)CN(C2CCC(=O)NC2=O)C3=O)c1-c1ccc(CN2CCOCC2)cc1] | 500.4 |
| 310 | 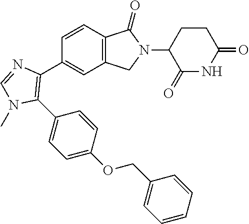 [SMILES: Cn1cnc(-c2ccc3c(c2)CN(C2CCC(=O)NC2=O)C3=O)c1-c1ccc(OCc2ccccc2)cc1] | 507.3 |
| 311 | 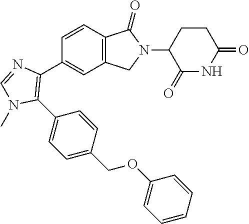 [SMILES: Cn1cnc(-c2ccc3c(c2)CN(C2CCC(=O)NC2=O)C3=O)c1-c1ccc(COc2ccccc2)cc1] | 507.3 |
| 312 | 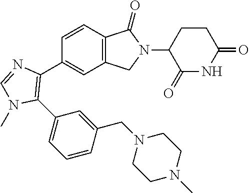 [SMILES: CN1CCN(Cc2cccc(-c3c(-c4ccc5c(c4)CN(C4CCC(=O)NC4=O)C5=O)ncn3C)c2)CC1] | 513.4 |
| 313 | 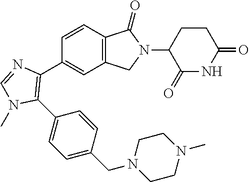 [SMILES: CN1CCN(Cc2ccc(-c3c(-c4ccc5c(c4)CN(C4CCC(=O)NC4=O)C5=O)ncn3C)cc2)CC1] | 513.4 |
| 314 | 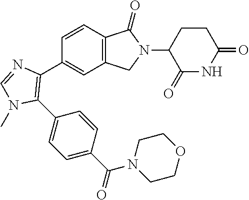 [SMILES: Cn1cnc(-c2ccc3c(c2)CN(C2CCC(=O)NC2=O)C3=O)c1-c1ccc(C(=O)N2CCOCC2)cc1] | 514.3 |
| 315 | 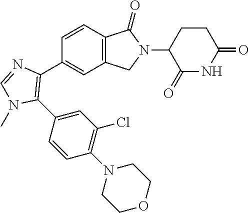 [SMILES: Cn1cnc(-c2ccc3c(c2)CN(C2CCC(=O)NC2=O)C3=O)c1-c1ccc(N2CCOCC2)c(Cl)c1] | 520.3 |
| 316 | 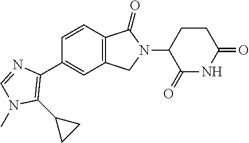 [SMILES: Cn1cnc(-c2ccc3c(c2)CN(C2CCC(=O)NC2=O)C3=O)c1C1CC1] | 365.2 |

### Examples 317-332

The compounds of Examples 317-332 were prepared according to the following Scheme 3:

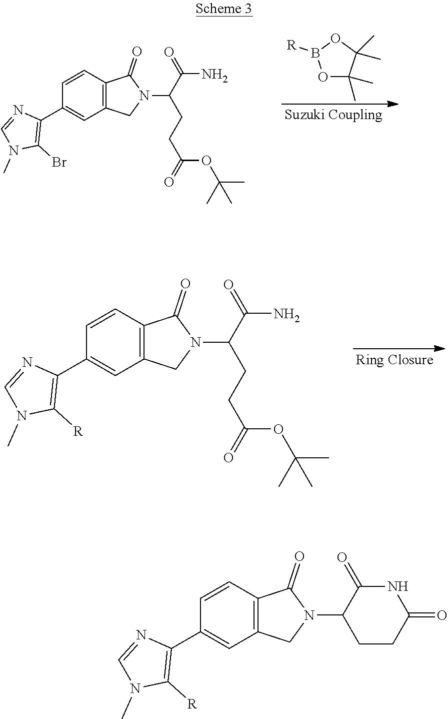

Step 1 Suzuki Coupling

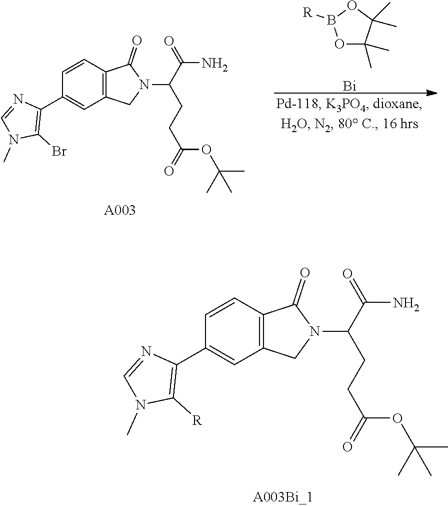

Conditions: To a vial containing a solution of A003 (150 μmol, 1.00 eq) and Bi (180 μmol, 1.20 eq) in Dioxane (1.50 mL) was added K₃PO₄ (1.2 M in H₂O, 450 μmol, 3.00 eq), and Pd-118 (15.0 μmol, 0.10 eq) under protection of N₂. The mixture was stirred at 65° C. for 16 hrs. The reaction mixture was diluted with H₂O (2.00 mL) and extracted with ethyl acetate (2.00 mL×3). The combined organic layers were washed with brine (2.00 mL), dried over Na₂SO₄, filtered and concentrated under reduced pressure to give crude intermediates used for next step.

Step 2 Ring Closure

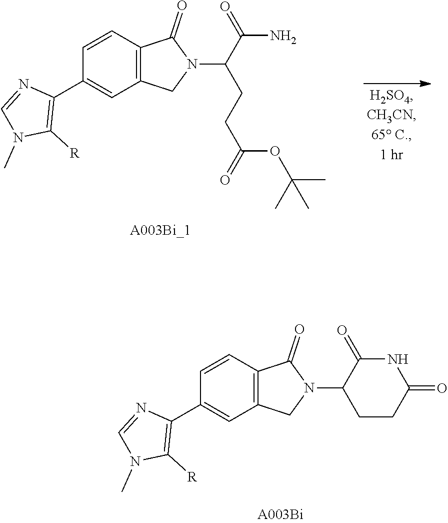

Conditions: To a vial containing a solution of A003Bi_1 (˜150 μmol, 1.00 eq) in ACN (1.50 mL), was added H₂SO₄ (150 μL). The mixture was stirred at 65° C. for 1 hr. The residue was concentrated under reduced pressure and purified by prep-HPLC to give desired product.

The following compounds were synthesized according to the above method.

| | | MW + 1 |
|---|---|---|
| Example | Structure | (Observed) |
| 317 | 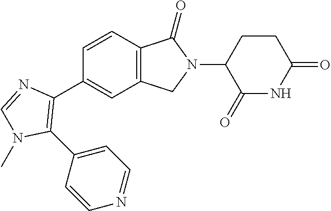 [SMILES: Cn1cnc(-c2ccc3c(c2)CN(C2CCC(=O)NC2=O)C3=O)c1-c1ccncc1] | 402.2 |
| 318 | 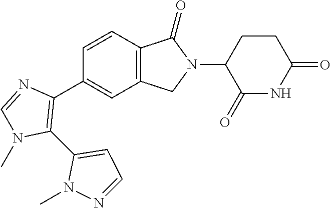 [SMILES: Cn1cnc(-c2ccc3c(c2)CN(C2CCC(=O)NC2=O)C3=O)c1-c1ccnn1C] | 405.2 |
| 319 | 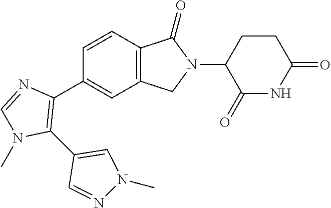 [SMILES: Cn1cc(-c2c(-c3ccc4c(c3)CN(C3CCC(=O)NC3=O)C4=O)ncn2C)cn1] | 405.3 |
| 320 | 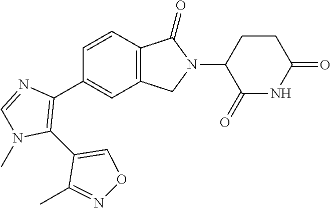 [SMILES: Cc1nocc1-c1c(-c2ccc3c(c2)CN(C2CCC(=O)NC2=O)C3=O)ncn1C] | 406.2 |
| 321 | 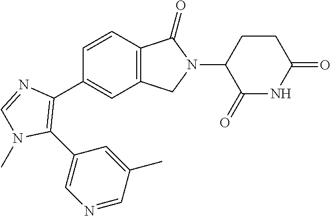 [SMILES: Cc1cncc(-c2c(-c3ccc4c(c3)CN(C3CCC(=O)NC3=O)C4=O)ncn2C)c1] | 416.2 |
| 322 | 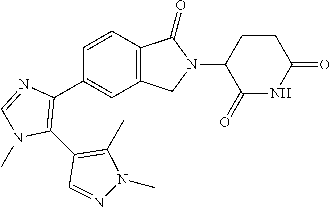 [SMILES: Cc1c(-c2c(-c3ccc4c(c3)CN(C3CCC(=O)NC3=O)C4=O)ncn2C)cnn1C] | 419.3 |
| 323 | 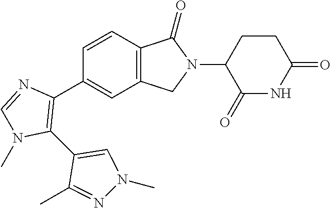 [SMILES: Cc1nn(C)cc1-c1c(-c2ccc3c(c2)CN(C2CCC(=O)NC2=O)C3=O)ncn1C] | 419.3 |
| 324 | 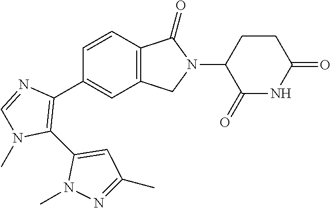 [SMILES: Cc1cc(-c2c(-c3ccc4c(c3)CN(C3CCC(=O)NC3=O)C4=O)ncn2C)n(C)n1] | 419.2 |
| 325 | 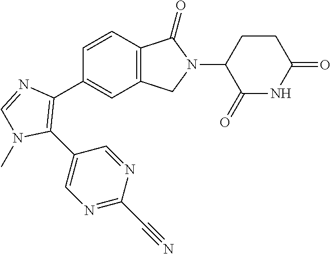 [SMILES: Cn1cnc(-c2ccc3c(c2)CN(C2CCC(=O)NC2=O)C3=O)c1-c1cnc(C#N)nc1] | 428.2 |
| 326 | 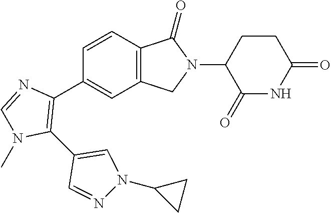 [SMILES: Cn1cnc(-c2ccc3c(c2)CN(C2CCC(=O)NC2=O)C3=O)c1-c1cnn(C2CC2)c1] | 431.3 |
| 327 | 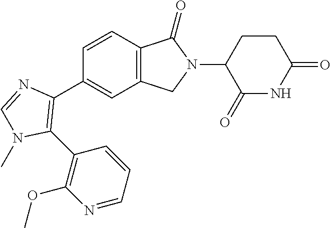 [SMILES: COc1ncccc1-c1c(-c2ccc3c(c2)CN(C2CCC(=O)NC2=O)C3=O)ncn1C] | 432.2 |
| 328 | 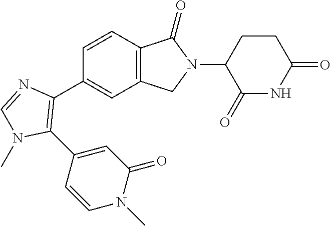 [SMILES: Cn1cnc(-c2ccc3c(c2)CN(C2CCC(=O)NC2=O)C3=O)c1-c1ccn(C)c(=O)c1] | 432.3 |
| 329 | 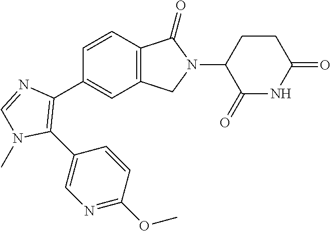 [SMILES: COc1ccc(-c2c(-c3ccc4c(c3)CN(C3CCC(=O)NC3=O)C4=O)ncn2C)cn1] | 432.3 |
| 330 | 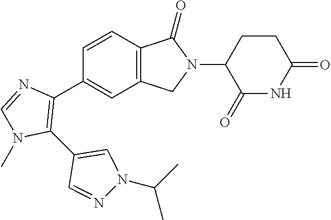 [SMILES: CC(C)n1cc(-c2c(-c3ccc4c(c3)CN(C3CCC(=O)NC3=O)C4=O)ncn2C)cn1] | 433.3 |
| 331 | 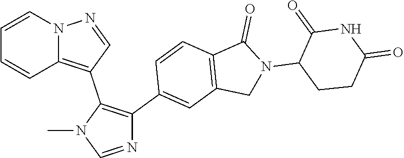 [SMILES: Cn1cnc(-c2ccc3c(c2)CN(C2CCC(=O)NC2=O)C3=O)c1-c1cnn2ccccc12] | 441.2 |
| 332 | 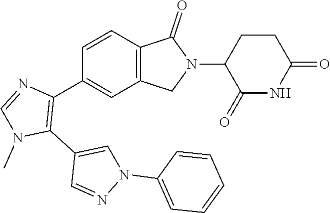 [SMILES: Cn1cnc(-c2ccc3c(c2)CN(C2CCC(=O)NC2=O)C3=O)c1-c1cnn(-c2ccccc2)c1] | 467.3 |

### Example 333

### Synthesis of 3-(1-oxo-5-(5-phenyl-1-(trifluoromethyl)-1H-pyrazol-4-yl)isoindolin-2-yl)piperidine-2,6-dione

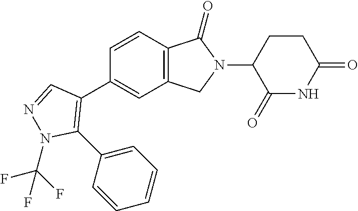 [SMILES: O=C1CCC(N2Cc3cc(-c4cnn(C(F)(F)F)c4-c4ccccc4)ccc3C2=O)C(=O)N1]

A. 4-bromo-5-phenyl-1-(trifluoromethyl)-1H-pyrazole: To a solution of 4-bromo-1-(trifluoromethyl)-1H-pyrazole (400 mg, 1.86 mmol, 1.00 eq) and bromobenzene (1.75 g, 11.1 mmol, 1.18 mL, 6.00 eq) in DMF (6.00 mL) was added K₂CO₃ (514 mg, 3.72 mmol, 2.00 eq), P(oxole)₃ (86.40 mg, 372 μmol, 0.20 eq) and Pd(OAc)₂ (125 mg, 558 μmol, 0.30 eq) under N₂. The mixture was stirred at 100° C. for 48 h under N₂. The reaction mixture was poured into H₂O (30.0 mL) and extracted with ethyl acetate (3×30.0 mL). The combined organic layer was washed with brine (3×30.0 mL), and dried over by Na₂SO₄, filtered and concentrate filtrate under reduced pressure to get residue. The residue was purified by preparative-TLC (Petroleum ether:Ethyl acetate=1:0, R_{f}=0.50) to give the title compound (130 mg, 393 μmol, 21.1% yield, 88.0% purity in LCMS at 220 nm) as a light yellow oil. (ESI⁺) m/z: 290.9 (M+H)⁺, (C₁₀H₆BrF₃N₂).

B. 3-(1-oxo-5-(5-phenyl-1-(trifluoromethyl)-1H-pyrazol-4-yl)isoindolin-2-yl)piperidine-2,6-dione: To a solution of 4-bromo-5-phenyl-1-(trifluoromethyl)-1H-pyrazole (100 mg, 343 μmol, 1.00 eq), 3-(1-oxo-5-(4,4,5,5-tetramethyl-1,3,2-dioxaborolan-2-yl)isoindolin-2-yl)piperidine-2,6-dione (317 mg, 858 μmol, 2.50 eq) and K₃PO₄ (218 mg, 1.03 mmol, 3.00 eq) in dioxane (5.00 mL) and H₂O (0.25 mL) was added Ru-Phos-Pd-G₃ (28.7 mg, 34.3 μmol, 0.10 eq) under N₂. The mixture was stirred at 100° C. for 3 h under N₂. The reaction mixture was concentrated under reduced pressure to get a residue at 45° C. The residue was purified by preparative-HPLC (using a Phenomenex luna C18 (150×25 mm×5 μm) and gradient of 37.0%-67.0% acetonitrile in water containing 0.05% FA over 10 min at a flow rate of 25 mL/min) to give the title compound (109 mg, 239 μmol, 69.5% yield, 99.8% purity in HPLC at 220 nm) as white solid. ¹H NMR: (400 MHz, DMSO-d₆) δ 10.9 (s, 1H), 8.45-8.41 (m, 1H), 7.60-7.51 (m, 4H), 7.46-7.45 (m, 3H), 7.28-7.26 (m, 1H), 5.09-5.05 (m, 1H), 4.38-4.19 (m, 2H), 2.92-2.84 (m, 1H), 2.54-2.51 (m, 1H), 2.39-2.35 (m, 1H), 1.98-1.95 (m, 1H). (ESI⁺) m/z: 455.1 (M+H)⁺, (C₂₃H₁₇F₃N₄O₃).

### Example 334

### Synthesis of 3-(5-(2-cyclohexyl-1-methyl-1H-imidazol-5-yl)-1-oxoisoindolin-2-yl)piperidine-2,6-dione

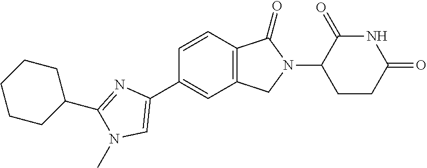 [SMILES: Cn1cc(-c2ccc3c(c2)CN(C2CCC(=O)NC2=O)C3=O)nc1C1CCCCC1]

A. 5-Bromo-2-cyclohexyl-1-methyl-1H-imidazole: To a solution of 2-cyclohexyl-1-methyl-1H-imidazole (0.10 g, 608 μmol, 1.00 eq) in ACN (1.00 mL) was added a solution of NBS (113 mg, 639 μmol, 1.05 eq) in ACN (1.00 mL) dropwise at 0° C. The reaction mixture was stirred at 25° C. for 2 h. The reaction mixture was added to H₂O (3.00 mL) and extracted with EtOAc (3×10.0 mL), dried over Na₂SO₄ and concentrated in vacuum to give a residue. The residue was purified by preparative-TLC (SiO₂, Dichloromethane:Methanol=20:1; TLC, Dichloromethane:Methanol=20:1, R_{f}=0.40) to give the title compound (120 mg, 493 μmol, 81.0% yield) as yellow solid. ¹H NMR: (400 MHz, CDCl₃) δ 6.92 (s, 1H), 3.53 (s, 3H), 2.66-2.60 (m, 1H), 1.88-1.85 (m, 2H), 1.74-1.72 (m, 1H), 1.67-1.61 (m, 3H), 1.40-1.34 (m, 4H). (ESI⁺) m/z: 243.0 (M+H)⁺, (C₁₀H₁₅BrN₂).

B. 3-(5-(2-cyclohexyl-1-methyl-1H-imidazol-5-yl)-1-oxoisoindolin-2-yl)piperidine-2,6-dione: To a solution of 5-bromo-2-cyclohexyl-1-methyl-1H-imidazole (100 mg, 411 μmol, 1.00 eq), 3-[1-oxo-5-(4,4,5,5-tetramethyl-1,3,2-dioxaborolan-2-yl)isoindolin-2-yl]piperidine-2,6-dione (228 mg, 616 μmol, 1.50 eq) in dioxane (1.00 mL) and H₂O (0.05 mL) was added Ru-Phos-Pd-G₃ (68.8 mg, 82.2 μmol, 0.20 eq), K₃PO₄ (174 mg, 822 μmol, 2.00 eq) under N₂. The reaction mixture was stirred at 100° C. for 2 h under N₂. The reaction mixture was concentrated in vacuum to give a residue. The residue was purified by preparative-HPLC (using a Phenomenex luna C18 (150 mm×25 mm×10 μm) and gradient of 7-37% acetonitrile in water containing 0.05% TFA over 10 min at a flow rate of 25 mL/min to give the title compound (96.6 mg, 241 μmol, 58.6% yield, 99.9% purity in HPLC at 220 nm) as white solid. ¹H NMR: (400 MHz, DMSO-d₆) δ 11.0 (s, 1H), 7.92-7.90 (m, 2H), 7.83 (s, 1H), 7.72-7.70 (m, 1H), 5.19-5.14 (m, 1H), 4.57-4.41 (m, 2H), 3.77 (s, 3H), 3.44-3.43 (m, 1H), 3.23-3.22 (m, 1H), 2.93-2.88 (m, 1H), 2.64-2.50 (m, 1H), 2.46-2.43 (m, 1H), 2.04-2.00 (m, 2H), 1.98-1.82 (m, 3H), 1.60-1.55 (m, 2H), 1.49-1.46 (m, 2H), 1.46-1.27 (m, 1H). (ESI⁺) m/z: 407.1 (M+H)⁺, (C₂₃H₂₆N₄O₃).

### Example 335

### Synthesis of 3-(5-(2-cyclohexyl-1-methyl-1H-imidazol-4-yl)-1-oxoisoindolin-2-yl)piperidine-2,6-dione

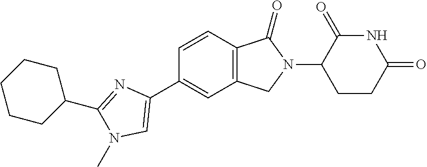 [SMILES: Cn1cc(-c2ccc3c(c2)CN(C2CCC(=O)NC2=O)C3=O)nc1C1CCCCC1]

A. 4,5-Dibromo-2-cyclohexyl-1-methyl-1H-imidazole: A solution of 2-cyclohexyl-1-methyl-1H-imidazole (0.25 g, 1.50 mmol, 1.00 eq) and NBS (0.55 g, 3.15 mmol, 2.10 eq) in DMF (3.00 mL) was stirred at 25° C. for 2 h. The reaction mixture was poured into H₂O (5.00 mL), then extracted with EtOAc (3×5.00 mL). The organic layers were combined, dried with Na₂SO₄ and concentrated in vacuum to give residue. The residue was purified by preparative-TLC (SiO₂, Dichloromethane:Methanol=20:1, R_{f}=0.30) to give the title compound (0.26 g, 0.80 mmol, 72.0% yield) as yellow solid. (ESI⁺) m/z: 320.2 (M+H)⁺, (C₁₀H₁₄Br₂N₂).

B. 4-Bromo-2-cyclohexyl-1-methyl-1H-imidazole: To a solution of 4,5-dibromo-2-cyclohexyl-1-methyl-1H-imidazole (0.26 g, 0.80 mmol, 1.00 eq) in THF (5.00 mL) was added EtMgBr (0.20 g, 1.60 mmol, 2.00 eq, 1.00 M in THF) at −20° C. under N₂. The reaction mixture was stirred at −20° C. for 4 h under N₂. The reaction mixture was quenched with saturated solution of NaHCO₃ (5.00 mL) and H₂O (3.00 mL). The aqueous phase was extracted with EtOAc (3×10.0 mL), dried over Na₂SO₄ and concentrated in vacuum to give a residue. The residue was purified by preparative-TLC (SiO₂, Dichloromethane:Methanol=20:1, R_{f}=0.20) to give the title compound (110 mg, 0.45 mmol, 57.7% yield) as yellow solid. ¹H NMR: (400 MHz, CDCl₃) δ 6.92 (s, 1H), 3.53 (s, 3H), 2.66-2.60 (m, 1H), 1.88-1.85 (m, 2H), 1.74-1.72 (m, 1H), 1.67-1.61 (m, 3H), 1.40-1.34 (m, 4H). (ESI⁺) m/z: 243.0 (M+H)⁺, (C₁₀H₁₅BrN₂).

C. 3-(5-(2-Cyclohexyl-1-methyl-1H-imidazol-4-yl)-1-oxoisoindolin-2-yl)piperidine-2,6-dione: To a solution of 4-bromo-2-cyclohexyl-1-methyl-imidazole (70.0 mg, 287 μmol, 1.00 eq), 3-[1-oxo-5-(4,4,5,5-tetramethyl-1,3,2-dioxaborolan-2-yl)isoindolin-2-yl]piperidine-2,6-dione (319 mg, 863 μmol, 3.00 eq) in dioxane (1.00 mL) and H₂O (0.05 mL) was added Ru-Phos-Pd-G₃ (48.1 mg, 57.5 μmol, 0.20 eq), K₃PO₄ (122 mg, 575 μmol, 2.00 eq) under N₂. The reaction mixture was stirred at 100° C. for 2 h under N₂. The reaction mixture was concentrated in vacuum to give a residue. The residue was purified by preparative-HPLC (using a Phenomenex luna C18 (150 mm×25 mm×5 μm) and gradient of 13-33% acetonitrile in water containing 0.05% FA over 10 min at a flow rate of 25 mL/min to give the title compound (26.8 mg, 64.4 μmol, 22.3% yield, 99.7% purity in HPLC at 220 nm) as white solid. ¹H NMR: (400 MHz, DMSO-d₆) δ 11.0 (s, 1H), 8.09 (s, 1H), 8.00 (s, 1H), 7.89-7.85 (m, 2H), 5.16-5.11 (m, 1H), 4.55-4.38 (m, 2H), 3.83 (s, 3H), 2.96-2.92 (m, 1H), 2.89-2.86 (m, 1H), 2.63-2.55 (m, 1H), 2.45-2.42 (m, 1H), 1.96-1.93 (m, 3H), 1.85-1.82 (m, 2H), 1.73-1.66 (m, 3H), 1.49-1.46 (m, 2H), 1.30-1.25 (m, 1H). (ESI⁺) m/z: 407.1 (M+H)⁺, (C₂₃H₂₆N₄O₃).

### Example 336

### Synthesis of 3-(5-(1-(2,2-difluoropropyl)-5-phenyl-1H-pyrazol-4-yl)-1-oxoisoindolin-2-yl)piperidine-2,6-dione

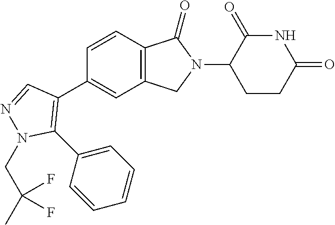 [SMILES: CC(F)(F)Cn1ncc(-c2ccc3c(c2)CN(C2CCC(=O)NC2=O)C3=O)c1-c1ccccc1]

A. 1-(4-Bromo-1H-pyrazol-1-yl)propan-2-one: To a solution of 4-bromo-1H-pyrazole (10.0 g, 68.0 mmol, 1.00 eq) in acetone (200 mL) was added 1-chloropropan-2-one (7.12 g, 76.9 mmol, 1.13 eq) and K₂CO₃ (28.2 g, 204 mmol, 3.00 eq) under N₂. The reaction mixture was stirred at 25° C. for 12 h under N₂. After the reaction was completed, the reaction mixture was quenched with H₂O (300 mL) and extracted with ethyl acetate (3×200 mL). The combined organic layer was washed with brine (3×200 mL), dried over Na₂SO₄, filtered and the filtrate was concentrated in vacuum to get a crude product. The crude product was purified by reversed-phase HPLC (0.1% TFA condition) to give the title compound (7.80 g, 38.4 mmol, 56.4% yield, 100% purity by LCMS at 220 nm) as a white solid. ¹H NMR: (400 MHz, DMSO-d₆) δ 7.87 (s, 1H), 7.57 (s, 1H), 5.12 (s, 2H), 2.10 (s, 3H). (ESI⁺) m/z: 202.9 (M+H)⁺, (C₆H₇BrN₂O).

B. 4-Bromo-1-(2,2-difluoropropyl)-1H-pyrazole: To a solution of 1-(4-bromopyrazol-1-yl)propan-2-one (7.80 g, 38.4 mmol, 1.00 eq) in DCM (80.0 mL) was added DAST (24.7 g, 153 mmol, 20.3 mL, 4.00 eq) at −78° C. under N₂. The reaction mixture was stirred at 20° C. for 5 h under N₂. After the reaction was completed, the reaction mixture was poured into H₂O (200 mL) and extracted with DCM (3×100 mL). The combined organic layers were washed with brine (100 mL), dried over Na₂SO₄, filtered and the filtrate was concentrated in vacuum to give the title compound (7.70 g, 32.3 mmol, 84.1% yield, 94.5% purity by LCMS at 220 nm) as yellow oil. ¹H NMR: (400 MHz, DMSO-d₆) δ 7.99 (s, 1H), 7.63 (s, 1H), 4.64 (t, J=13.2 Hz, 2H), 1.59 (t, J=19.2 Hz, 3H). (ESI⁺) m/z: 224.9 (M+H)⁺, (C₆H₇BrF₂N₂).

C. 4-Bromo-1-(2,2-difluoropropyl)-5-phenyl-1H-pyrazole: To a solution of 4-bromo-1-(2,2-difluoropropyl)pyrazole (2.00 g, 8.89 mmol, 1.00 eq) in DMF (40.0 mL) was added bromobenzene (8.37 g, 53.3 mmol, 5.62 mL, 6.00 eq), P(oxole)₃ (412 mg, 1.78 mmol, 0.20 eq), K₂CO₃ (2.46 g, 17.7 mmol, 2.00 eq) and Pd(OAc)₂ (199 mg, 888 μmol, 0.10 eq) under N₂. The mixture was stirred at 100° C. for 12 h under N₂. After the reaction was completed, the reaction mixture was cooled to 25° C. and filtered. The filtrate was poured into H₂O (50.0 mL) and extracted with ethyl acetate (3×50.0 mL). The organic layer was washed with brine (50.0 mL), dried over Na₂SO₄, filtered and the filtrate was concentrated in vacuum to give a residue. The residue was purified by column chromatography (SiO₂, Petroleum ether:Ethyl acetate=10:1 to 5:1, R_{f}=0.40 (Petroleum ether:Ethyl acetate=5:1)) to give the title compound (1.10 g, 2.22 mmol, 25.0% yield, 60.9% purity by LCMS at 220 nm) as yellow oil. (ESI⁺) m/z: 301.0 (M+H)⁺, (C₁₂H₁₁BrF₂N₂).

D. tert-Butyl 5-amino-4-(5-(1-(2,2-difluoropropyl)-5-phenyl-1H-pyrazol-4-yl)-1-oxoisoindolin-2-yl)-5-oxopentanoate: To a solution of 4-bromo-1-(2,2-difluoropropyl)-5-phenyl-pyrazole (300 mg, 996 μmol, 1.00 eq) in dioxane (5.00 mL) and H₂O (0.250 mL) was added tert-butyl 5-amino-5-oxo-4-[1-oxo-5-(4,4,5,5-tetramethyl-1,3,2-dioxaborolan-2-yl)isoindolin-2-yl]pentanoate (885 mg, 1.99 mmol, 2.00 eq), Ru-Phos-Pd-G₃ (83.3 mg, 99.6 μmol, 0.10 eq) and K₃PO₄ (422 mg, 1.99 mmol, 2.00 eq) under N₂. The reaction mixture was stirred at 100° C. for 2 h under N₂. After the reaction was completed, the reaction mixture was filtered and the filtrate was poured into H₂O (10.0 mL) and extracted with ethyl acetate (3×10.0 mL). The organic layer was washed with brine (30.0 mL), dried over Na₂SO₄, filtered and the filtrate was concentrated in vacuum to give a residue. The residue was purified by preparative-TLC (Dichloromethane:Methanol=10:1, R_{f}=0.40) to give the title compound (170 mg, 276 μmol, 27.7% yield, 87.5% purity by LCMS at 220 nm) as yellow oil. (ESI⁺) m/z: 539.2 (M+H)⁺, (C₂₉H₃₂F₂N₄O₄).

E. 3-(5-(1-(2,2-Difluoropropyl)-5-phenyl-1H-pyrazol-4-yl)-1-oxoisoindolin-2-yl)piperidine-2,6-dione: To a solution of tert-butyl 5-amino-4-[5-[1-(2,2-difluoropropyl)-5-phenyl-pyrazol-4-yl]-1-oxo-isoindolin-2-yl]-5-oxo-pentanoate (170 mg, 276 μmol, 1.00 eq) in ACN (3.00 mL) was added TsOH (475 mg, 2.76 mmol, 10.0 eq) under N₂. The reaction mixture was stirred at 80° C. for 4 h under N₂. After the reaction was completed, the reaction mixture was filtered and the filtrate was concentrated under vacuum to give a residue. The residue was purified by preparative-TLC (SiO₂, Dichloromethane:Methanol=10:1, R_{f}=0.39) and by prep-HPLC (using a Phenomenex Luna C18 (150 mm×25 mm 10 μm) and gradient of 26-56% acetonitrile in water containing 0.5% TFA over 10 min at a flow rate of 25.0 mL/min) to give the title compound (35.9 mg, 77.4 μmol, 28.0% yield, 100% purity by HPLC at 220 nm) as a white solid. ¹H NMR: (400 MHz, DMSO-d₆) δ 11.0 (s, 1H), 8.05 (s, 1H), 7.56-7.51 (m, 4H), 7.38-7.34 (m, 3H), 7.22 (dd, J=8.0, 1.2 Hz, 1H), 5.07 (dd, J=13.6, 5.2 Hz, 1H), 4.47 (t, J=12.8 Hz, 2H), 4.26 (dd, J=58.4, 17.2 Hz, 2H), 2.94-2.86 (m, 1H), 2.59 (s, 1H), 2.38 (d, J=4.4 Hz, 1H), 1.98-1.94 (m, 1H), 1.59 (t, J=19.2 Hz, 3H). (ESI⁺) m/z: 465.1 (M+H)⁺, (C₂₅H₂₂F₂N₄O₃).

### Example 337

### Synthesis of 3-(5-(1-(difluoromethyl)-5-phenyl-1H-pyrazol-3-yl)-1-oxoisoindolin-2-yl)piperidine-2,6-dione

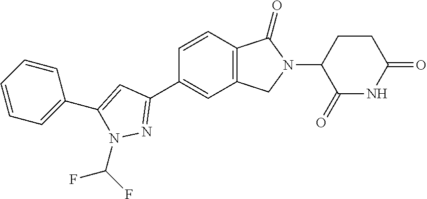 [SMILES: O=C1CCC(N2Cc3cc(-c4cc(-c5ccccc5)n(C(F)F)n4)ccc3C2=O)C(=O)N1]

A. 3-Bromo-5-phenyl-1H-pyrazole: To a solution of 3-bromo-5-phenyl-1-(tetrahydro-2H-pyran-2-yl)-1H-pyrazole (1.00 g, 3.26 mmol, 1.00 eq) in HCl/dioxane (4 M, 5.00 mL, 6.14 eq) at 25° C. The mixture was stirred at 25° C. for 24 h. The reaction mixture was concentrated under reduced pressure to get a residue at 40° C. The reaction mixture was dissolved in Dichloromethane (60.0 mL). The combined organic layer was washed with saturated NaHCO₃ solution (3×60.0 mL), dried over by Na₂SO₄, filtered and concentrated. The residue was purified by column chromatography (SiO₂, Petroleum ether:Ethyl acetate=40:1 to 10:1, TLC:Petroleum ether:Ethyl acetate=5:1, R=0.30) to give the title compound (700 mg, 3.06 mmol, 94.0% yield, 97.6% purity in LCMS at 220 nm) as a white solid. (ESI⁺) m/z: 222.9 (M+H)⁺, (C₉H₇BrN₂).

B. 3-Bromo-1-(difluoromethyl)-5-phenyl-1H-pyrazole): To a solution of 3-bromo-5-phenyl-1H-pyrazole (110 mg, 491 μmol, 1.00 eq) and KF (57.3 mg, 986 μmol, 23.1 μL, 2.00 eq) in ACN (4.00 mL). The mixture was stirred until a colorless oil was formed. Then diethyl (bromodifluoromethyl) phosphonate (131 mg, 493 μmol, 1.00 eq) was added to the mixture at 25° C. under N₂. The mixture was stirred at 25° C. for 12 h under N₂. The reaction mixture was concentrated under reduced pressure to get a residue at 43° C. The residue was purified by preparative-TLC (Petroleum ether:Ethyl acetate=20:1,R_{f}=0.50). to give the title compound (130 mg, 476 μmol, 19.3% yield, 100% purity in LCMS at 220 nm, Batche 5) as a light yellow oil. ¹HNMR: (400 MHz, DMSO-d₆) δ 7.84-7.69 (m, 1H), 7.56-7.50 (m, 5H), 6.88 (s, 1H). (ESI⁺) m/z: 272.8 (M+H)⁺, (C₁₀H₇BrF₂N₂)

C. 3-(5-(1-(Difluoromethyl)-5-phenyl-1H-pyrazol-3-yl)-1-oxoisoindolin-2-yl)piperidine-2,6-dione: To a solution of 3-bromo-1-(difluoromethyl)-5-phenyl-1H-pyrazole (130 mg, 476 μmol, 1.00 eq), 3-(1-oxo-5-(4,4,5,5-tetramethyl-1,3,2-dioxaborolan-2-yl)isoindolin-2-yl)piperidine-2,6-dione (352 mg, 952 μmol, 2.00 eq) and K₃PO₄ (303 mg, 1.43 mmol, 3.00 eq) in dioxane (5.00 mL) and H₂O (0.25 mL) was added Ru-Phos-Pd-G₃ (39.8 mg, 47.6 μmol, 0.10 eq) under N₂. The mixture was stirred at 100° C. for 2 h under N₂. The reaction mixture was concentrated under reduced pressure to get a residue at 45° C. The residue was purified by preparative-HPLC (using a Phenomenex luna C18 (150×25 mm×10 μm) and gradient of 34.0%-64.0% acetonitrile in water containing 0.05% FA over 10 min at a flow rate of 25 mL/min to give the title compound (89.1 mg, 204 μmol, 42.8% yield, 99.9% purity in HPLC at 220 nm) as an off-white solid. ¹HNMR: (400 MHz, DMSO-d₆) δ 11.0-10.9 (m, 1H), 8.19-8.08 (m, 2H), 7.84-7.79 (m, 2H), 7.64-7.54 (m, 5H), 7.31 (s, 1H), 5.19-5.12 (m, 1H), 4.57-4.39 (m, 2H), 2.96-2.89 (m, 1H), 2.63-2.60 (m, 1H), 2.45-2.41 (m, 1H), 2.09-2.01 (m, 1H). (ESI⁺) m/z: 437.0 (M+H)⁺, (C₂₃H₁₈F₂N₄O₃).

### Example 338

### Synthesis of 3-(5-(1,5-dimethyl-2-phenyl-1H-imidazol-4-yl)-1-oxoisoindolin-2-yl)piperidine-2,6-dione

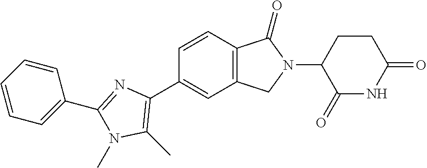 [SMILES: Cc1c(-c2ccc3c(c2)CN(C2CCC(=O)NC2=O)C3=O)nc(-c2ccccc2)n1C]

A. 1,5-dimethyl-2-phenyl-1H-imidazole: To a solution of 5-methyl-2-phenyl-1H-imidazole (5.00 g, 31.6 mmol, 1.00 eq) in THF (50.0 mL) was added 18-crown-6 (83.5 mg, 316 μmol, 0.01 eq) and t-BuOK (3.55 g, 31.6 mmol, 1.00 eq) at 25° C. A solution of CH₃I (4.49 g, 31.6 mmol, 1.97 mL, 1.00 eq) in THF (90.0 mL) was added dropwise at 25° C. The mixture was stirred at 25° C. for 12 h. The reaction mixture was cooled to 0° C., and extracted with DCM (3×50.0 mL). Then the organic layer was dried by Na₂SO₄, filtered and concentrated to get residue. The residue was purified by column chromatography (SiO₂, Dichloromethane:Ethyl acetate=100:1, TLC:Dichloromethane:Ethyl acetate=100:1, R_{f}=0.40) to give the title compound (360 mg, 2.09 mmol, 6.6% yield, in LCMS at 220 nm) as light yellow solid. ¹H NMR: (400 MHz, DMSO-d₆) δ 7.61-7.59 (m, 2H), 7.48-7.40 (m, 3H), 6.74 (s, 1H), 3.56 (s, 3H), 2.22 (s, 3H) (ESI⁺) m/z: 173 (M+H)⁺, (C₁₁H₁₂N₂).

B. 4-Iodo-1, 5-dimethyl-2-phenyl-1H-imidazole: To a solution of H₂SO₄ (8.00 mL) in H₂O (5.00 mL) was added 12 (760 mg, 2.99 mmol, 603 μL, 5.16 e-1.00 eq), acetic acid (8.40 g, 140 mmol, 8.01 mL, 24.1 eq), iodic acid (260 mg, 1.48 mmol, 2.55e-1 eq) and 1,5-dimethyl-2-phenyl-1H-imidazole (1.00 g, 5.81 mmol, 1.00 eq) at 25° C. Then the mixture was stirred at 80° C. for 4 h. The reaction mixture was neutralized with NaOH aq, and poured into saturated sodium thiosulfate solution (3×100 mL) and extracted with chloroform (100 mL), the organic layer was separated. Combined organic layers were dried over MgSO₄, filtered and concentrated under reduced pressure to give a residue. The residue was purified by column chromatography (SiO₂, Dichloromethane:Ethyl acetate=40:1, TLC:Dichloromethane:Ethyl acetate=40:1, R_{f}=0.70) to give the title compound (200 mg, 630 μmol, 10.8% yield, 94.0% purity in LCMS at 220 nm) as a white solid. ¹H NMR: (400 MHz, DMSO-d₆) δ 7.62-7.59 (m, 2H), 7.50-7.42 (m, 3H), 3.62-3.60 (m, 3H), 2.14 (s, 3H) (ESI⁺) m/z: 299.0 (M+H)⁺, (C₁₁H₁₁IN₂).

C. 3-(5-(1,5-Dimethyl-2-phenyl-1H-imidazol-4-yl)-1-oxoisoindolin-2-yl)piperidine-2,6-dione: To a solution of 4-iodo-1,5-dimethyl-2-phenyl-1H-imidazole (180 mg, 603 μmol, 1.00 eq), 3-(1-oxo-5-(4,4,5,5-tetramethyl-1,3,2-dioxaborolan-2-yl)isoindolin-2-yl)piperidine-2,6-dione (670 mg, 1.81 mmol, 3.00 eq) and K₃PO₄ (384 mg, 1.81 mmol, 3.00 eq) in dioxane (6.00 mL) and H₂O (0.30 mL) was added Ru-Phos-Pd-G₃ (50.5 mg, 60.3 μmol, 0.10 eq) under N₂. The mixture was stirred at 100° C. for 5 h under N₂. The reaction mixture was concentrated under reduced pressure to get a residue at 45° C. The residue was purified by preparative-HPLC (using a Phenomenex luna C18 (150×25 mm×5 μm) and gradient of 8.00%-38.0% acetonitrile in water containing 0.05% FA over 3 min at a flow rate of 25 mL/min to give the title compound (22.1 mg, 52.0 μmol, 8.64% yield, 97.7% purity in HPLC at 220 nm) as white solid. ¹H NMR: (400 MHz, DMSO-d₆) δ 11.0-10.9 (m, 1H), 7.91-7.81 (m, 5H), 7.72-7.62 (m, 3H), 5.18-5.13 (m, 1H), 4.57-4.40 (m, 2H), 3.76 (s, 3H), 2.97-2.90 (m, 1H), 2.64-2.60 (m, 1H), 2.54 (s, 3H), 2.46-2.41 (m, 1H), 2.05-2.02 (m, 1H). (ESI⁺) m/z: 415.2 (M+H)⁺, (C₂₄H₂₂N₄O₃).

### Example 339

### Synthesis of 3-(5-(5H-imidazo[5,1-a]isoindol-1-yl)-1-oxoisoindolin-2-yl)piperidine-2,6-dione

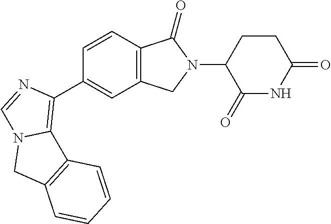 [SMILES: O=C1CCC(N2Cc3cc(-c4ncn5c4-c4ccccc4C5)ccc3C2=O)C(=O)N1]

A. 1-(2-Iodobenzyl)-1H-imidazole: To a solution of NaH (1.35 g, 33.6 mmol, 13.4 mL, 60.0% purity, 2.00 eq) in THF (50.0 mL) was added imidazole (1.15 g, 16.8 mmol, 1.00 eq) at 0° C. under N₂. The reaction mixture was stirred at 0° C. for 1 h under N₂. After the evolution of hydrogen stopped, 1-(bromomethyl)-2-iodo-benzene (5.00 g, 16.8 mmol, 1.00 eq) was added and stirred at 25° C. for 11 h under N₂. After the reaction was completed, the reaction mixture was poured into a mixture of 100 mL of saturated NH₄Cl solution. The aqueous layer was extracted with ethyl acetate (3×50.0 mL) and the combined organic layer was dried over Na₂SO₄, concentrated in vacuum to get a crude product. The crude product was purified by column chromatography (SiO₂, Dichloromethane:Methanol=10:1, R_{f}=0.50 (Dichloromethane:Methanol=10:1)) to give the title compound (2.00 g, 6.92 mmol, 41.7% yield, 98.6% purity in LCMS at 220 nm) as a white solid. ¹H NMR: (400 MHz, DMSO-d₆) δ 7.91 (d, J=7.6 Hz, 1H), 7.74 (s, 1H), 7.38 (t, J=7.6 Hz, 1H), 7.17 (s, 1H), 7.09 (t, J=6.8 Hz, 1H), 6.95 (s, 1H), 6.88 (d, J=8.0 Hz, 1H), 5.21 (s, 2H), (ESI⁺) m/z: 284.9 (M+H)⁺, (C₁₀H₉IN₂).

B. 5H-Imidazo[5,1-a]isoindole: To a solution of 1-[(2-iodophenyl)methyl]-imidazole (2.00 g, 7.04 mmol, 1.00 eq) in DMSO (50.0 mL) was added K₂CO₃ (1.95 g, 14.0 mmol, 2.00 eq), PPh₃ (184 mg, 703 μmol, 0.10 eq) and Pd(OAc)₂ (79.0 mg, 351 μmol, 0.05 eq) under N₂. The reaction mixture was stirred at 140° C. for 1 h under N₂. After the reaction was completed, the reaction mixture was filtered and the filter cake was washed with Methanol (3×30.0 mL), H₂O (2×30.0 mL) and Methanol (30.0 mL). Then the filtrate was concentrated under vacuum to give the title compound (500 mg, 3.20 mmol, 45.4% yield) as yellow solid. (ESI⁺) m/z: 157.0 (M+H)⁺, (C₁₀H₈N₂).

C. 1-Bromo-5H-imidazo[5,1-a]isoindole: To a solution of 5H-imidazo[5,1-a]isoindole (100 mg, 640 μmol, 1.00 eq) in ACN (2.00 mL) was added NBS (119 mg, 672 μmol, 1.05 eq) at 0° C. under N₂. The reaction mixture was stirred at 25° C. for 1 h under N₂.

After the reaction was completed, the reaction mixture was poured to H₂O (10.0 mL) and extracted with ethyl acetate (3×15.0 mL). The combined organic layer was washed with brine (3×15.0 mL), dried over Na₂SO₄, filtered and the filtrate was concentrated in vacuum to give the title compound (125 mg, 531 μmol, 83.0% yield) as a yellow solid. ¹H NMR: (400 MHz, DMSO-d₆) δ 7.90 (s, 1H), 7.61 (d, J=7.6 Hz, 1H), 7.54 (d, J=7.6 Hz, 1H), 7.42 (t, J=7.6 Hz, 1H), 7.34 (t, J=6.4 Hz, 1H), 5.18 (s, 2H). (ESI⁺) m/z: 234.9 (M+H)⁺, (C₁₀H₇BrN₂).

D. 3-(5-(5H-Imidazo[5,1-a]isoindol-1-yl)-1-oxoisoindolin-2-yl)piperidine-2,6-dione: To a solution of 1-bromo-5H-imidazo[5,1-a]isoindole (100 mg, 425 μmol, 1.00 eq) in dioxane (4.00 mL) and H₂O (0.20 mL) was added 3-[1-oxo-5-(4,4,5,5-tetramethyl-1,3,2-dioxaborolan-2-yl)isoindolin-2-yl]piperidine-2,6-dione (393 mg, 1.06 mmol, 2.50 eq), K₃PO₄ (180 mg, 850 μmol, 2.00 eq) and Ru-Phos-Pd-G₃ (35.5 mg, 42.5 μmol, 0.10 eq) under N₂. The reaction mixture was stirred at 100° C. for 2 h under N₂. After the reaction was completed, the reaction mixture was filtered and the filtrate was concentrated under vacuum to give a residue. The residue was purified by preparative-TLC (Dichloromethane:Methanol=10:1, R_{f}=0.40) and by preparative-HPLC (using a Phenomenex Luna C18 (150 mm×25 mm×10 μm) and gradient of 8-38% acetonitrile in water containing 0.5% TFA over 10 min at a flow rate of 25.0 mL/min) to give the title compound (16.3 mg, 41.0 μmol, 9.64% yield, 100% purity in HPLC at 220 nm) as yellow solid. ¹H NMR: (400 MHz, DMSO-d₆) δ 11.0 (s, 1H), 8.85-8.75 (m, 1H), 8.03 (s, 1H), 7.95 (t, J=5.6 Hz, 3H), 7.68 (d, J=7.2 Hz, 1H), 7.49 (t, J=7.2 Hz, 2H), 5.41 (s, 2H), 5.17 (dd, J=12.8, 4.8 Hz, 1H), 4.55 (dd, J=53.6, 17.2 Hz, 2H), 2.99-2.90 (m, 1H), 2.61 (d, J=0.8 Hz, 1H), 2.46-2.41 (m, 1H), 2.07-2.05 (m, 1H). (ESI⁺) m/z: 399.1 (M+H)⁺, (C₂₃H₁₈N₄O₃).

### Example 340

### Synthesis of 3-(5-(1-methyl-5-(4-(trifluoromethoxy)phenyl)-1H-pyrazol-4-yl)-1-oxoisoindolin-2-yl)piperidine-2,6-dione

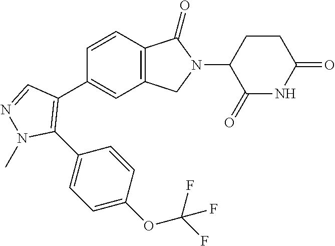 [SMILES: Cn1ncc(-c2ccc3c(c2)CN(C2CCC(=O)NC2=O)C3=O)c1-c1ccc(OC(F)(F)F)cc1]

A. 4-Iodo-1-methyl-5-(4-(trifluoromethoxy)phenyl)-1H-pyrazole: To a solution of 4-iodo-1-methyl-pyrazole (500 mg, 2.40 mmol, 1.00 eq) and 1-bromo-4-(trifluoromethoxy)benzene (1.74 g, 7.21 mmol, 1.07 mL, 3.00 eq) in DMF (10.0 mL) was added Pd(OAc)₂ (53.9 mg, 240 μmol, 0.10 eq), P(oxole)₃ (111 mg, 480 μmol, 0.20 eq) and K₂CO₃ (664 mg, 4.81 mmol, 2.00 eq). The mixture was stirred at 100° C. for 12 h under N₂. After the reaction was completed, the reaction mixture was filtered and the filtrate was poured into 30.0 mL of water and extracted with EtOAc (3×30.0 mL). The combined organic layers were washed with brine (3×10.0 mL), dried over Na₂SO₄, filtered and the filtrate was concentrated in vacuum to get a residue. The residue was purified by reversed-phase HPLC (0.1% HCl condition) to give the title compound (50.0 mg, 113 μmol, 4.74% yield, 83.9% purity by LCMS at 220 nm) as a brown solid. ¹H NMR: (400 MHz, DMSO-d₆) δ 7.59 (s, 1H), 7.43 (d, J=8.4 Hz, 2H), 7.36 (d, J=8.4 Hz, 2H), 3.85 (s, 3H). (ESI⁺) m/z: 368.9 (M+H)⁺, (C₁₁H₈F₃IN₂O).

B. 3-(5-(1-Methyl-5-(4-(trifluoromethoxy)phenyl)-1H-pyrazol-4-yl)-1-oxoisoindolin-2-yl)piperidine-2,6-dione: To a solution of 4-iodo-1-methyl-5-[4-(trifluoromethoxy)phenyl]pyrazole (50.0 mg, 135 μmol, 1.00 eq) in dioxane (2.00 mL) and H₂O (0.10 mL) was added 3-[1-oxo-5-(4,4,5,5-tetramethyl-1,3,2-dioxaborolan-2-yl)isoindolin-2-yl]piperidine-2,6-dione (125 mg, 339 μmol, 2.50 eq), Ru-Phos-Pd-G₃ (11.3 mg, 13.5 μmol, 0.100 eq) and K₃PO₄ (57.6 mg, 271 μmol, 2.00 eq). The reaction mixture was stirred at 100° C. for 2 h under N₂. After the reaction was completed, the reaction mixture was filtered and the filtrate was concentrated under vacuum to give a residue. The residue was purified by preparative-TLC (Dichloromethane:Methanol=10:1, R_{f}=0.50) and by preparative-HPLC (using a Welch Xtimate C18 (150 mm×25 mm 5 μm) and gradient of 32-62% acetonitrile in water containing 0.5% TFA over 10 min at a flow rate of 25.0 mL/min) to give the title compound (21.8 mg, 44.7 μmol, 32.9% yield, 99.3% purity by HPLC at 220 nm) as a white solid. ¹H NMR: (400 MHz, DMSO-d₆) δ 11.0 (s, 1H), 7.90 (s, 1H), 7.59-7.51 (m, 5H), 7.37 (s, 1H), 7.20 (d, J=8.8 Hz, 1H), 5.07 (dd, J=13.2, 5.2 Hz, 1H), 4.28 (dd, J=55.6, 17.6 Hz, 2H), 3.74 (s, 3H), 2.94-2.85 (m, 1H), 2.59 (s, 1H), 2.38 (d, J=4.4 Hz, 1H), 1.99-1.95 (m, 1H). (ESI⁺) m/z: 484.1 (M+H)⁺, (C₂₄H₁₉F₃N₄O₄).

### Example 341

### Synthesis of 3-(5-(1-(difluoromethyl)-3-(3-fluoro-4-methoxyphenyl)-1H-pyrazol-4-yl)-1-oxoisoindolin-2-yl)piperidine-2,6-dione

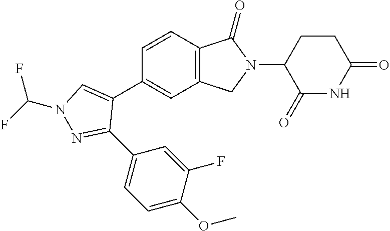 [SMILES: COc1ccc(-c2nn(C(F)F)cc2-c2ccc3c(c2)CN(C2CCC(=O)NC2=O)C3=O)cc1F]

A. 5-(3-Fluoro-4-methoxyphenyl)-1H-pyrazole: To a solution of 5-bromo-1H-pyrazole (5.00 g, 34.0 mmol, 1.00 eq), (3-fluoro-4-methoxyphenyl)boronic acid (6.36 g, 37.4 mmol, 1.10 eq) and K₃PO₄ (14.4 g, 68.0 mmol, 2.00 eq) in dioxane (50.0 mL) and H₂O (5.0 mL) was added PdCl₂(dtbpf) (2.22 g, 3.40 mmol, 0.10 eq) under N₂. The mixture was stirred at 90° C. for 12 h under N₂. Then the mixture was filtered to collect liquid and the liquid was washed with H₂O (100 mL) and extracted with Ethyl acetate (3×80.0 mL). The combined organic layer was washed with saturated NaCl aqueous (2×100 mL), dried over by Na₂SO₄ and concentrated under reduced pressure to get residue. The residue was purified by column chromatography (SiO₂, Petroleum ether:Ethyl acetate=1:2, R_{f}=0.30) to get title compound (4.60 g, 20.2 mmol, 59.5% yield, 84.6% purity in LCMS at 220 nm) as yellow solid. (ESI⁺) m/z: 193.1 (M+H)⁺, (C₁₀H₉FN₂O).

B. 4-Bromo-5-(3-fluoro-4-methoxyphenyl)-1H-pyrazole: To a solution of 5-(3-fluoro-4-methoxyphenyl)-1H-pyrazole (2.00 g, 10.4 mmol, 1.00 eq) in DMF (20.0 mL) was added NBS (1.85 g, 10.41 mmol, 1.00 eq). The mixture was stirred at 20° C. for 2 h. Then the mixture was poured into H₂O (100 mL) and extracted with Ethyl acetate (3×80.0 mL). The combined organic layer was washed with saturated NaCl aqueous (3×80.0 mL), dried over Na₂SO₄ and concentrated under reduced pressure to get residue. The residue was purified by column chromatography (SiO₂, Petroleum ether:Ethyl acetate=1:1, R_{f}=0.40) to get title compound (2.30 g, 8.48 mmol, 76.6% yield, 100% purity in LCMS at 220 nm) as yellow solid. (ESI⁺) m/z: 270.9 (M+H)⁺, (C₁₀H₈BrFN₂O).

C. 4-Bromo-1-(difluoromethyl)-3-(3-fluoro-4-methoxyphenyl)-1H-pyrazole: To a solution of 4-bromo-5-(3-fluoro-4-methoxyphenyl)-1H-pyrazole (500 mg, 1.84 mmol, 1.00 eq) and KF (214 mg, 3.69 mmol, 86.4 μL, 2.00 eq) in ACN (13.0 mL) was stirred until a colorless oil was formed, diethyl (bromodifluoromethyl)phosphonate (492 mg, 1.84 mmol, 1.00 eq) was added to the mixture subsequently. Then the mixture was stirred at 25° C. for 12 h. The residue was purified by column chromatography (SiO₂, Petroleum ether:Ethyl acetate=2:1, R_{f}=0.50) to get title compound (360 mg, 986 μmol, 53.4% yield, 88% purity in LCMS at 220 nm) as yellow solid. ¹H NMR: (400 MHz, DMSO-d₆) δ 8.67 (s, 1H), 7.68-7.65 (m, 1H), 7.63-7.60 (m, 2H), 7.36-7.26 (m, 1H), 3.89 (s, 3H). (ESI⁺) m/z: 320.7 (M+H)⁺, (C₁₁H₈BrF₃N₂O).

D. 3-(5-(1-(Difluoromethyl)-3-(3-fluoro-4-methoxyphenyl)-1H-pyrazol-4-yl)-1-oxoisoindolin-2-yl)piperidine-2,6-dione: To a solution of 3-(1-oxo-5-(4,4,5,5-tetramethyl-1,3,2-dioxaborolan-2-yl)isoindolin-2-yl)piperidine-2,6-dione (346 mg, 934 μmol, 2.00 eq), 4-bromo-1-(difluoromethyl)-3-(3-fluoro-4-methoxyphenyl)-1H-pyrazole (150 mg, 467 μmol, 1.00 eq) and K₃PO₄ (198 mg, 934 μmol, 2.00 eq) in dioxane (2.00 mL) and H₂O (0.10 mL) was added Ru-Phos-Pd-G₃ (78.1 mg, 93.4 μmol, 0.20 eq) under N₂. The mixture was stirred at 100° C. for 2 h under N₂. The mixture was filtered to collect liquid and concentrated under reduced pressure to get residue. The crude product was purified by preparative-HPLC (using a Phenomenex luna C18 (150 mm×25 mm 10 μm) and gradient of 35%-65% acetonitrile in water containing 0.05% FA over 15 min at a flow rate of 25 mL/min) to give the title compound (64.3 mg, 131 μmol, 28.1% yield, 98.9% purity in HPLC at 220 nm) as a yellow solid. ¹H NMR: (400 MHz, DMSO-d₆) δ 10.9 (s, 1H), 8.58 (s, 1H), 7.89-7.73 (m, 1H), 7.71-7.57 (m, 1H), 7.40-7.38 (m, 1H), 7.27-7.24 (m, 1H), 7.19-7.09 (m, 3H), 5.14-5.10 (m, 1H), 4.47-4.30 (m, 2H), 3.84 (s, 3H), 2.95-2.88 (m, 1H), 2.62-2.58 (m, 1H), 2.42-2.38 (m, 1H), 2.03-2.00 (m, 1H). (ESI⁺) m/z: 485.0 (M+H)⁺, (C₂₄H₁₉F₃N₄O₄).

### Example 342

The following compound was synthesized according to the procedure of Scheme 1 as shown in Examples 55-116.

| | | MW + 1 | Step 1 |
|---|---|---|---|
| Example | Structure | (Observed) | Conditions |
| 342 | 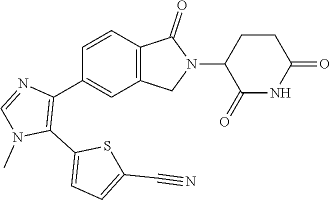 [SMILES: Cn1cnc(-c2ccc3c(c2)CN(C2CCC(=O)NC2=O)C3=O)c1-c1ccc(C#N)s1] | 432.2 | 2 |

### Examples 343-359

The following compounds were synthesized according to the procedure of Scheme 2 as shown in Examples 161-204.

| Ex- | | MW + 1 |
|---|---|---|
| am- | | (Ob- |
| ple | Structure | served) |
| 343 | 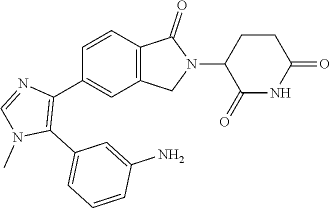 [SMILES: Cn1cnc(-c2ccc3c(c2)CN(C2CCC(=O)NC2=O)C3=O)c1-c1cccc(N)c1] | 416.2 |
| 344 | 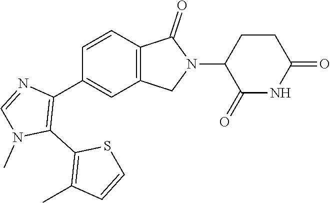 [SMILES: Cc1ccsc1-c1c(-c2ccc3c(c2)CN(C2CCC(=O)NC2=O)C3=O)ncn1C] | 421.2 |
| 345 | 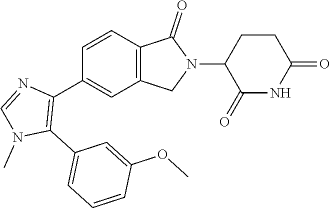 [SMILES: COc1cccc(-c2c(-c3ccc4c(c3)CN(C3CCC(=O)NC3=O)C4=O)ncn2C)c1] | 431.3 |
| 346 | 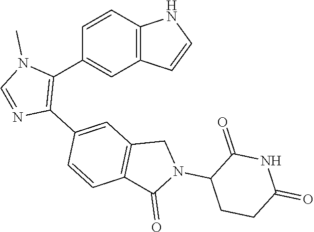 [SMILES: Cn1cnc(-c2ccc3c(c2)CN(C2CCC(=O)NC2=O)C3=O)c1-c1ccc2[nH]ccc2c1] | 440.2 |
| 347 | 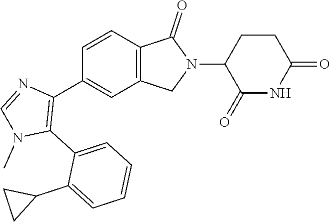 [SMILES: Cn1cnc(-c2ccc3c(c2)CN(C2CCC(=O)NC2=O)C3=O)c1-c1ccccc1C1CC1] | 441.3 |
| 348 | 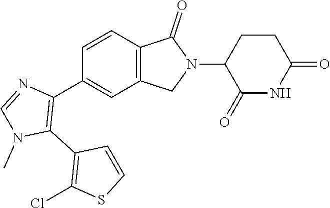 [SMILES: Cn1cnc(-c2ccc3c(c2)CN(C2CCC(=O)NC2=O)C3=O)c1-c1ccsc1Cl] | 441.2 |
| 349 | 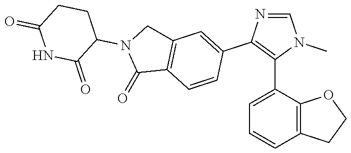 [SMILES: Cn1cnc(-c2ccc3c(c2)CN(C2CCC(=O)NC2=O)C3=O)c1-c1cccc2c1OCC2] | 443.3 |
| 350 | 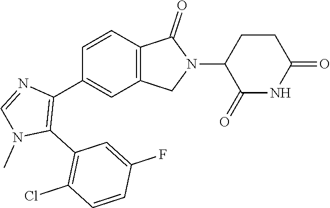 [SMILES: Cn1cnc(-c2ccc3c(c2)CN(C2CCC(=O)NC2=O)C3=O)c1-c1cc(F)ccc1Cl] | 453.2 |
| 351 | 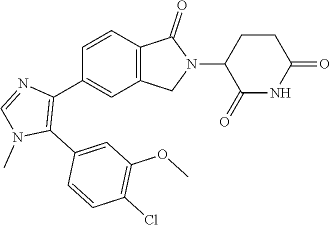 [SMILES: COc1cc(-c2c(-c3ccc4c(c3)CN(C3CCC(=O)NC3=O)C4=O)ncn2C)ccc1Cl] | 465.2 |
| 352 | 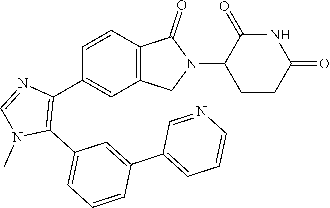 [SMILES: Cn1cnc(-c2ccc3c(c2)CN(C2CCC(=O)NC2=O)C3=O)c1-c1cccc(-c2cccnc2)c1] | 478.3 |
| 353 | 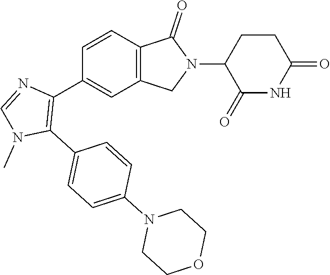 [SMILES: Cn1cnc(-c2ccc3c(c2)CN(C2CCC(=O)NC2=O)C3=O)c1-c1ccc(N2CCOCC2)cc1] | 486.3 |
| 354 | 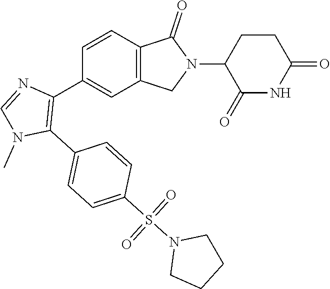 [SMILES: Cn1cnc(-c2ccc3c(c2)CN(C2CCC(=O)NC2=O)C3=O)c1-c1ccc(S(=O)(=O)N2CCCC2)cc1] | 534.3 |
| 355 | 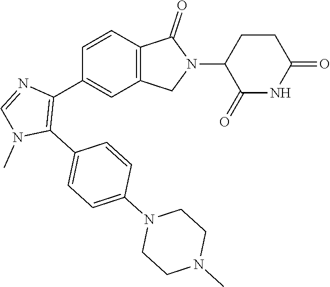 [SMILES: CN1CCN(c2ccc(-c3c(-c4ccc5c(c4)CN(C4CCC(=O)NC4=O)C5=O)ncn3C)cc2)CC1] | 499.4 |
| 356 | 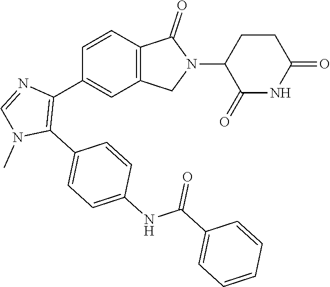 [SMILES: Cn1cnc(-c2ccc3c(c2)CN(C2CCC(=O)NC2=O)C3=O)c1-c1ccc(NC(=O)c2ccccc2)cc1] | 520.3 |
| 357 | 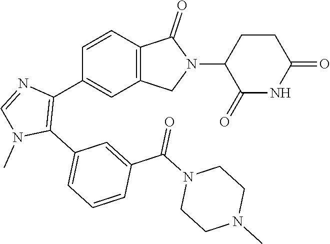 [SMILES: CN1CCN(C(=O)c2cccc(-c3c(-c4ccc5c(c4)CN(C4CCC(=O)NC4=O)C5=O)ncn3C)c2)CC1] | 527.3 |
| 358 | 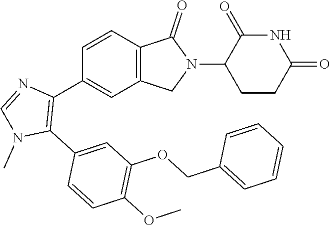 [SMILES: COc1ccc(-c2c(-c3ccc4c(c3)CN(C3CCC(=O)NC3=O)C4=O)ncn2C)cc1OCc1ccccc1] | 537.3 |
| 359 | 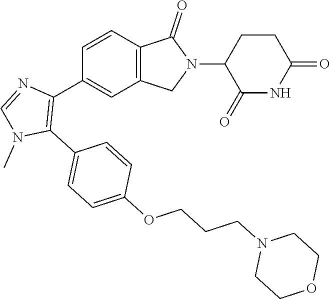 [SMILES: Cn1cnc(-c2ccc3c(c2)CN(C2CCC(=O)NC2=O)C3=O)c1-c1ccc(OCCCN2CCOCC2)cc1] | 544.4 |

### Examples 360-396

The compounds of Examples 360-396 were prepared according to the following

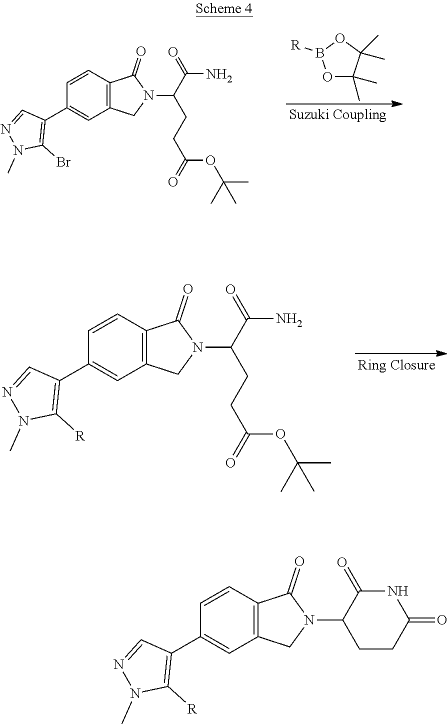

Step 1: Suzuki Coupling

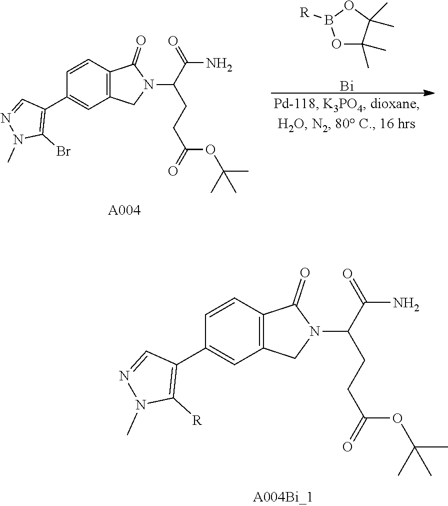

Conditions: To a vial containing a solution of A004 (100 μmol, 1.00 eq) and Bi (120 μmol, 1.20 eq) in Dioxane (1.00 mL) was added K₃PO₄ (1.2 M in H₂O, 300 μmol, 3.00 eq), and Pd-118 (10.0 μmol, 0.10 eq) under protection of N₂. The mixture was stirred at 65° C. for 16 hrs. The reaction mixture was diluted with H₂O (2.00 mL) and extracted with ethyl acetate (2.00 mL×3). The combined organic layers were washed with brine (2.00 mL), dried over Na₂SO₄, filtered and concentrated under reduced pressure to give crude intermediates used for next step.

Step 2: Ring Closure

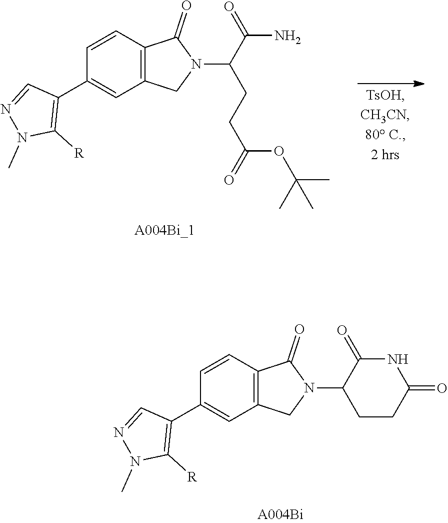

Conditions: To a vial containing a solution of A004Bi_1 (˜100 μmol, 1.00 eq) in ACN (1.00 mL), was added TsOH (1.00 mmol, 10.0 eq). The mixture was stirred at 80° C. for 2 hrs. The residue was concentrated under reduced pressure and purified by prep-HPLC to give final product.

| | | MW + |
|---|---|---|
| Ex- | | 1 |
| am- | | (Ob- |
| ple | Structure | served) |
| 360 | 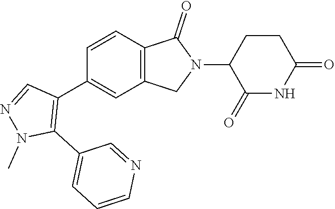 [SMILES: Cn1ncc(-c2ccc3c(c2)CN(C2CCC(=O)NC2=O)C3=O)c1-c1cccnc1] | 402.2 |
| 361 | 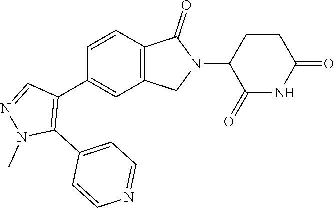 [SMILES: Cn1ncc(-c2ccc3c(c2)CN(C2CCC(=O)NC2=O)C3=O)c1-c1ccncc1] | 402.2 |
| 362 | 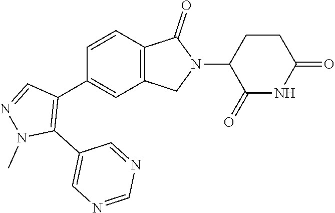 [SMILES: Cn1ncc(-c2ccc3c(c2)CN(C2CCC(=O)NC2=O)C3=O)c1-c1cncnc1] | 403.2 |
| 363 | 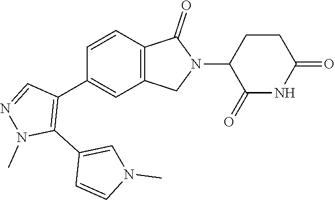 [SMILES: Cn1ccc(-c2c(-c3ccc4c(c3)CN(C3CCC(=O)NC3=O)C4=O)cnn2C)c1] | 404.2 |
| 364 | 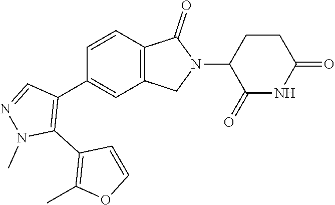 [SMILES: Cc1occc1-c1c(-c2ccc3c(c2)CN(C2CCC(=O)NC2=O)C3=O)cnn1C] | 405.2 |
| 365 | 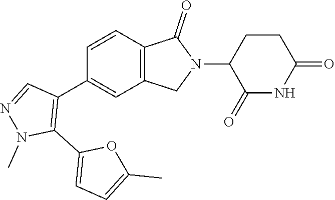 [SMILES: Cc1ccc(-c2c(-c3ccc4c(c3)CN(C3CCC(=O)NC3=O)C4=O)cnn2C)o1] | 405.2 |
| 366 | 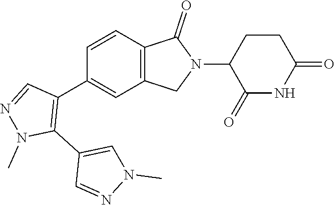 [SMILES: Cn1cc(-c2c(-c3ccc4c(c3)CN(C3CCC(=O)NC3=O)C4=O)cnn2C)cn1] | 405.2 |
| 367 | 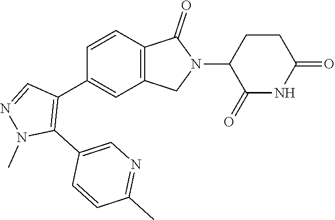 [SMILES: Cc1ccc(-c2c(-c3ccc4c(c3)CN(C3CCC(=O)NC3=O)C4=O)cnn2C)cn1] | 416.2 |
| 368 | 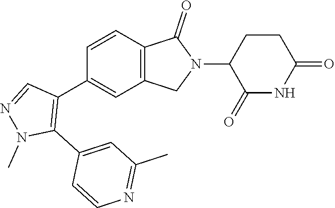 [SMILES: Cc1cc(-c2c(-c3ccc4c(c3)CN(C3CCC(=O)NC3=O)C4=O)cnn2C)ccn1] | 416.2 |
| 369 | 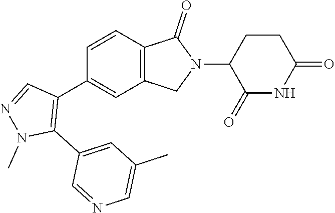 [SMILES: Cc1cncc(-c2c(-c3ccc4c(c3)CN(C3CCC(=O)NC3=O)C4=O)cnn2C)c1] | 416.2 |
| 370 | 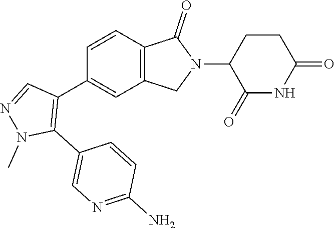 [SMILES: Cn1ncc(-c2ccc3c(c2)CN(C2CCC(=O)NC2=O)C3=O)c1-c1ccc(N)nc1] | 417.2 |
| 371 | 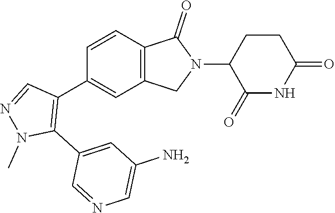 [SMILES: Cn1ncc(-c2ccc3c(c2)CN(C2CCC(=O)NC2=O)C3=O)c1-c1cncc(N)c1] | 417.2 |
| 372 | 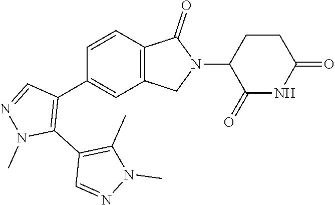 [SMILES: Cc1c(-c2c(-c3ccc4c(c3)CN(C3CCC(=O)NC3=O)C4=O)cnn2C)cnn1C] | 419.2 |
| 373 | 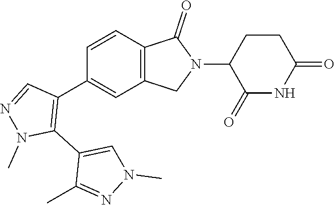 [SMILES: Cc1nn(C)cc1-c1c(-c2ccc3c(c2)CN(C2CCC(=O)NC2=O)C3=O)cnn1C] | 419.3 |
| 374 | 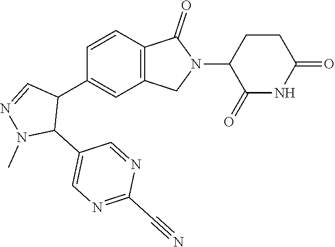 [SMILES: CN1N=CC(c2ccc3c(c2)CN(C2CCC(=O)NC2=O)C3=O)C1c1cnc(C#N)nc1] | 428.2 |
| 375 | 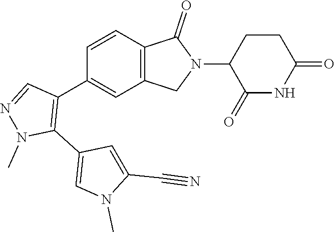 [SMILES: Cn1cc(-c2c(-c3ccc4c(c3)CN(C3CCC(=O)NC3=O)C4=O)cnn2C)cc1C#N] | 429.2 |
| 376 | 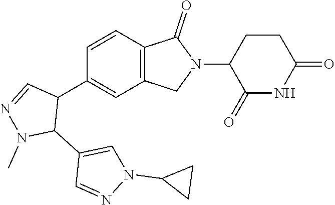 [SMILES: CN1N=CC(c2ccc3c(c2)CN(C2CCC(=O)NC2=O)C3=O)C1c1cnn(C2CC2)c1] | 431.2 |
| 377 | 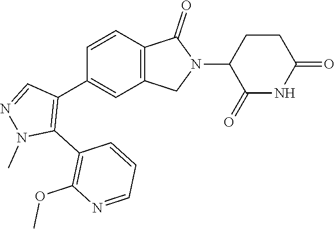 [SMILES: COc1ncccc1-c1c(-c2ccc3c(c2)CN(C2CCC(=O)NC2=O)C3=O)cnn1C] | 432.2 |
| 378 | 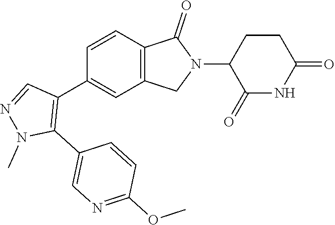 [SMILES: COc1ccc(-c2c(-c3ccc4c(c3)CN(C3CCC(=O)NC3=O)C4=O)cnn2C)cn1] | 432.2 |
| 379 | 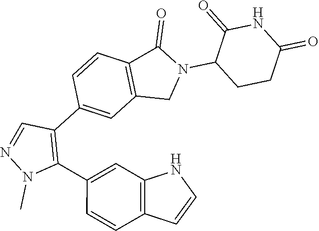 [SMILES: Cn1ncc(-c2ccc3c(c2)CN(C2CCC(=O)NC2=O)C3=O)c1-c1ccc2cc[nH]c2c1] | 440.2 |
| 380 | 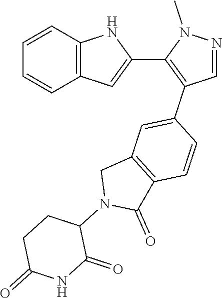 [SMILES: Cn1ncc(-c2ccc3c(c2)CN(C2CCC(=O)NC2=O)C3=O)c1-c1cc2ccccc2[nH]1] | 440.3 |
| 381 | 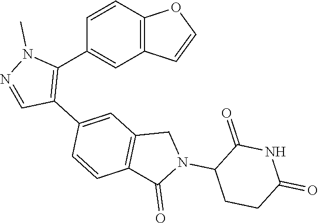 [SMILES: Cn1ncc(-c2ccc3c(c2)CN(C2CCC(=O)NC2=O)C3=O)c1-c1ccc2occc2c1] | 441.2 |
| 382 | 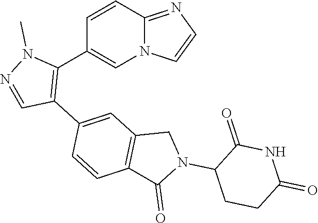 [SMILES: Cn1ncc(-c2ccc3c(c2)CN(C2CCC(=O)NC2=O)C3=O)c1-c1ccc2nccn2c1] | 441.2 |
| 383 | 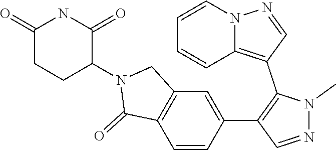 [SMILES: Cn1ncc(-c2ccc3c(c2)CN(C2CCC(=O)NC2=O)C3=O)c1-c1cnn2ccccc12] | 441.2 |
| 384 | 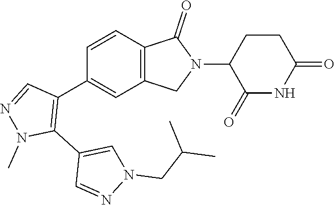 [SMILES: CC(C)Cn1cc(-c2c(-c3ccc4c(c3)CN(C3CCC(=O)NC3=O)C4=O)cnn2C)cn1] | 447.3 |
| 385 | 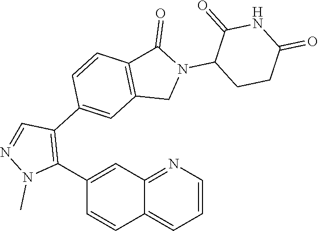 [SMILES: Cn1ncc(-c2ccc3c(c2)CN(C2CCC(=O)NC2=O)C3=O)c1-c1ccc2cccnc2c1] | 452.3 |
| 386 | 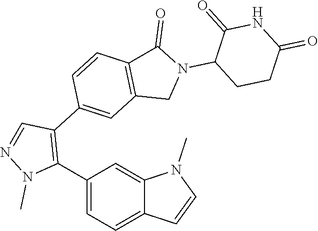 [SMILES: Cn1ncc(-c2ccc3c(c2)CN(C2CCC(=O)NC2=O)C3=O)c1-c1ccc2ccn(C)c2c1] | 454.2 |
| 387 | 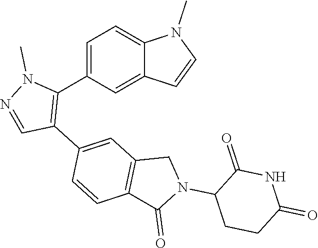 [SMILES: Cn1ncc(-c2ccc3c(c2)CN(C2CCC(=O)NC2=O)C3=O)c1-c1ccc2c(ccn2C)c1] | 454.3 |
| 388 | 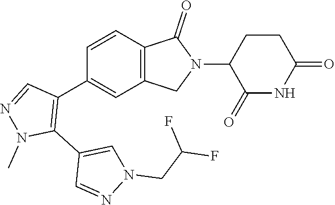 [SMILES: Cn1ncc(-c2ccc3c(c2)CN(C2CCC(=O)NC2=O)C3=O)c1-c1cnn(CC(F)F)c1] | 455.2 |
| 389 | 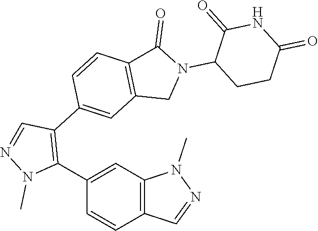 [SMILES: Cn1ncc(-c2ccc3c(c2)CN(C2CCC(=O)NC2=O)C3=O)c1-c1ccc2cnn(C)c2c1] | 455.3 |
| 390 | 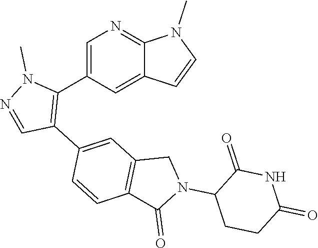 [SMILES: Cn1ncc(-c2ccc3c(c2)CN(C2CCC(=O)NC2=O)C3=O)c1-c1cnc2c(ccn2C)c1] | 455.3 |
| 391 | 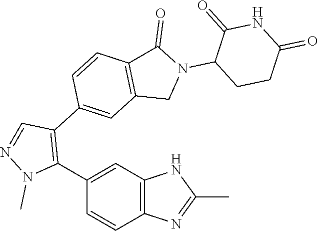 [SMILES: Cc1nc2ccc(-c3c(-c4ccc5c(c4)CN(C4CCC(=O)NC4=O)C5=O)cnn3C)cc2[nH]1] | 455.3 |
| 392 | 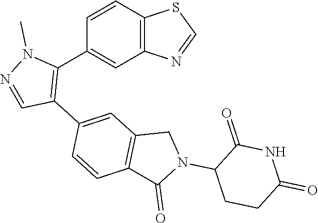 [SMILES: Cn1ncc(-c2ccc3c(c2)CN(C2CCC(=O)NC2=O)C3=O)c1-c1ccc2scnc2c1] | 458.2 |
| 393 | 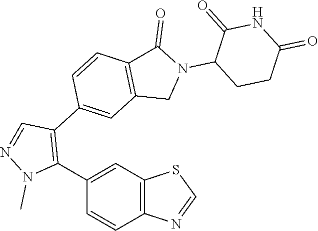 [SMILES: Cn1ncc(-c2ccc3c(c2)CN(C2CCC(=O)NC2=O)C3=O)c1-c1ccc2ncsc2c1] | 458.2 |
| 394 | 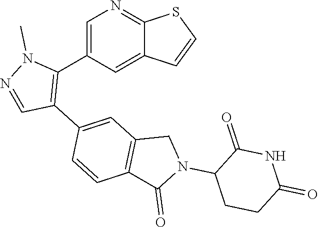 [SMILES: Cn1ncc(-c2ccc3c(c2)CN(C2CCC(=O)NC2=O)C3=O)c1-c1cnc2sccc2c1] | 458.2 |
| 395 | 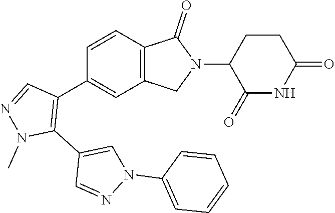 [SMILES: Cn1ncc(-c2ccc3c(c2)CN(C2CCC(=O)NC2=O)C3=O)c1-c1cnn(-c2ccccc2)c1] | 467.3 |
| 396 | 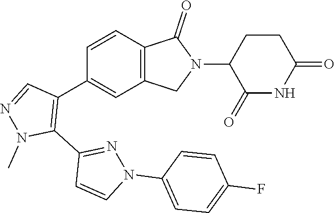 [SMILES: Cn1ncc(-c2ccc3c(c2)CN(C2CCC(=O)NC2=O)C3=O)c1-c1ccn(-c2ccc(F)cc2)n1] | 485.3 |

### Examples 397-407

The following compounds were synthesized according to the procedure of Scheme 3 as shown in Examples 317-332.

| | | MW + |
|---|---|---|
| Ex- | | 1 |
| am- | | (Ob- |
| ple | Structure | served) |
| 397 | 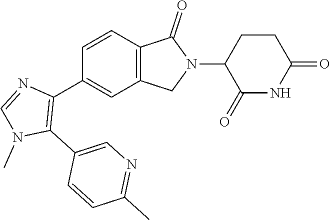 [SMILES: Cc1ccc(-c2c(-c3ccc4c(c3)CN(C3CCC(=O)NC3=O)C4=O)ncn2C)cn1] | 416.3 |
| 398 | 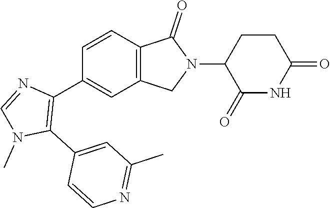 [SMILES: Cc1cc(-c2c(-c3ccc4c(c3)CN(C3CCC(=O)NC3=O)C4=O)ncn2C)ccn1] | 416.2 |
| 399 | 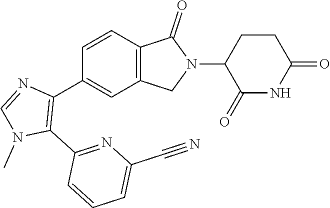 [SMILES: Cn1cnc(-c2ccc3c(c2)CN(C2CCC(=O)NC2=O)C3=O)c1-c1cccc(C#N)n1] | 427.2 |
| 400 | 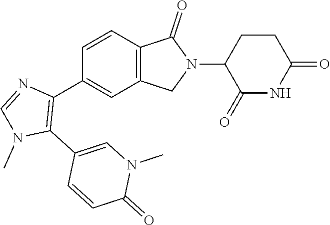 [SMILES: Cn1cnc(-c2ccc3c(c2)CN(C2CCC(=O)NC2=O)C3=O)c1-c1ccc(=O)n(C)c1] | 432.2 |
| 401 | 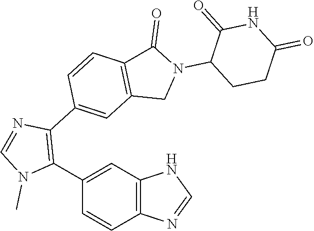 [SMILES: Cn1cnc(-c2ccc3c(c2)CN(C2CCC(=O)NC2=O)C3=O)c1-c1ccc2nc[nH]c2c1] | 441.2 |
| 402 | 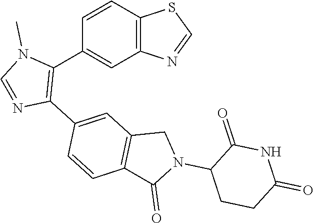 [SMILES: Cn1cnc(-c2ccc3c(c2)CN(C2CCC(=O)NC2=O)C3=O)c1-c1ccc2scnc2c1] | 458.2 |
| 403 | 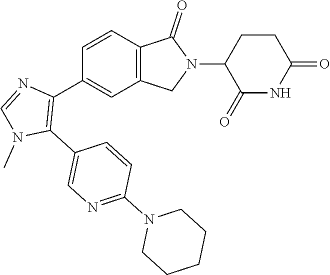 [SMILES: Cn1cnc(-c2ccc3c(c2)CN(C2CCC(=O)NC2=O)C3=O)c1-c1ccc(N2CCCCC2)nc1] | 485.3 |
| 404 | 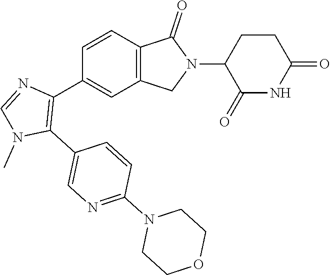 [SMILES: Cn1cnc(-c2ccc3c(c2)CN(C2CCC(=O)NC2=O)C3=O)c1-c1ccc(N2CCOCC2)nc1] | 487.3 |
| 405 | 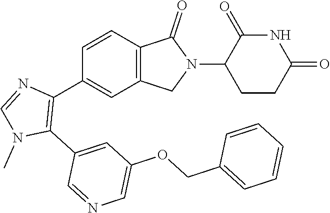 [SMILES: Cn1cnc(-c2ccc3c(c2)CN(C2CCC(=O)NC2=O)C3=O)c1-c1cncc(OCc2ccccc2)c1] | 508.3 |
| 406 | 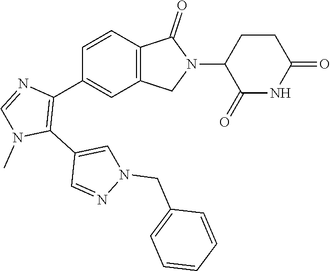 [SMILES: Cn1cnc(-c2ccc3c(c2)CN(C2CCC(=O)NC2=O)C3=O)c1-c1cnn(Cc2ccccc2)c1] | 481.3 |
| 407 | 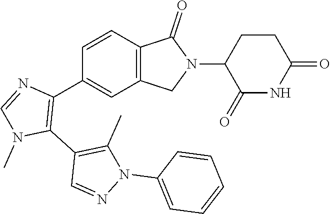 [SMILES: Cc1c(-c2c(-c3ccc4c(c3)CN(C3CCC(=O)NC3=O)C4=O)ncn2C)cnn1-c1ccccc1] | 481.3 |

### Examples 408-414

The following compounds were synthesized according to the procedure of Scheme 1 as shown in Examples 55-116.

| | | MW + 1 | Step 1 |
|---|---|---|---|
| Example | Structure | (Observed) | Conditions |
| 408 | 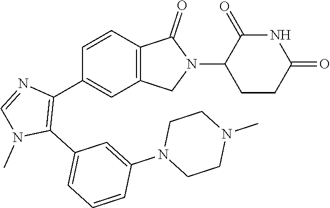 [SMILES: CN1CCN(c2cccc(-c3c(-c4ccc5c(c4)CN(C4CCC(=O)NC4=O)C5=O)ncn3C)c2)CC1] | 499.3 | 1 |
| 409 | 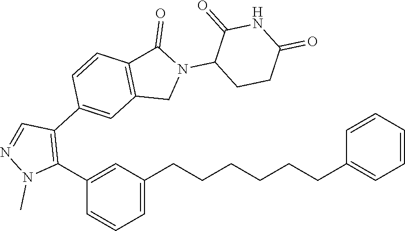 [SMILES: Cn1ncc(-c2ccc3c(c2)CN(C2CCC(=O)NC2=O)C3=O)c1-c1cccc(CCCCCCc2ccccc2)c1] | 561.4 | 1 |
| 410 | 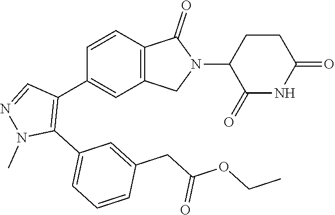 [SMILES: CCOC(=O)Cc1cccc(-c2c(-c3ccc4c(c3)CN(C3CCC(=O)NC3=O)C4=O)cnn2C)c1] | 487.3 | 1 |
| 411 | 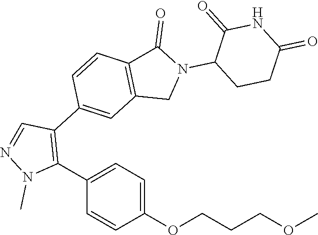 [SMILES: COCCCOc1ccc(-c2c(-c3ccc4c(c3)CN(C3CCC(=O)NC3=O)C4=O)cnn2C)cc1] | 489.3 | 1 |
| 412 | 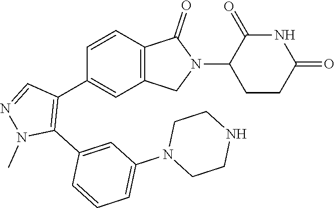 [SMILES: Cn1ncc(-c2ccc3c(c2)CN(C2CCC(=O)NC2=O)C3=O)c1-c1cccc(N2CCNCC2)c1] | 485.3 | 1 |
| 413 | 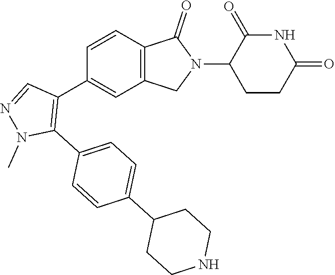 [SMILES: Cn1ncc(-c2ccc3c(c2)CN(C2CCC(=O)NC2=O)C3=O)c1-c1ccc(C2CCNCC2)cc1] | 484.3 | 1 |
| 414 | 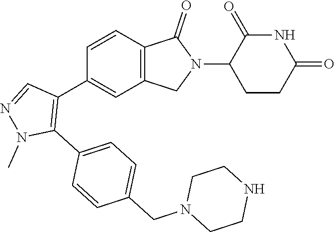 [SMILES: Cn1ncc(-c2ccc3c(c2)CN(C2CCC(=O)NC2=O)C3=O)c1-c1ccc(CN2CCNCC2)cc1] | 499.3 | 1 |

### Example 415

### Synthesis of 3-(5-(2-(1,3-dioxan-2-yl)-1-methyl-1H-imidazol-4-yl)-1-oxoisoindolin-2-yl) piperidine-2,6-dione

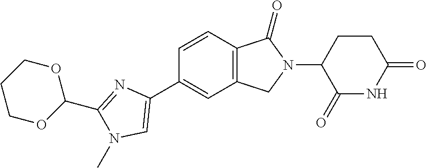 [SMILES: Cn1cc(-c2ccc3c(c2)CN(C2CCC(=O)NC2=O)C3=O)nc1C1OCCCO1]

A. 4-Bromo-2-(1,3-dioxan-2-yl)-1-methyl-1H-imidazole: To a solution of 4-bromo-1-methyl-imidazole-2-carbaldehyde (500 mg, 2.65 mmol, 1.00 eq) and propane-1,3-diol (402 mg, 5.29 mmol, 382 μL, 2.00 eq) in toluene (15.0 mL) was added TsOH (136 mg, 793 μmol, 0.30 eq). The mixture was stirred at 110° C. in a Dean-Stark trap apparatus for 12 h. Then the reaction mixture was concentrated in vacuum to get a residue. The residue was purified by reversed-phase HPLC (0.1% FA condition) to give the title compound (290 mg, 1.03 mmol, 39.0% yield, 88.0% purity in LCMS at 220 nm) as brown oil. (ESI⁺) m/z: 247.0 (M+H)⁺, (C₈H₁₁BrN₂₀₂).

B. 3-(5-(2-(1,3-Dioxan-2-yl)-1-methyl-1H-imidazol-4-yl)-1-oxoisoindolin-2-yl)piperidine-2,6-dione (3HCl): To a solution of 4-bromo-2-(1,3-dioxan-2-yl)-1-methyl-imidazole (140 mg, 498 μmol, 1.00 eq) and 3-[1-oxo-5-(4,4,5,5-tetramethyl-1,3,2-dioxaborolan-2-yl)isoindolin-2-yl]piperidine-2,6-dione (369 mg, 997 μmol, 2.00 eq) in dioxane (5.00 mL) and H₂O (0.25 mL) was added Ru-Phos-Pd-G₃ (41.7 mg, 49.8 μmol, 0.10 eq) and K₃PO₄ (211 mg, 997 μmol, 2.00 eq). The mixture was stirred at 100° C. for 4 h under N₂. Then the reaction mixture was filtered and the filtrate was concentrated in vacuum to get a residue. The residue was purified by preparative-TLC (SiO₂, DCM:Methanol=10:1, R_{f}=0.70) and then by preparative-HPLC (using a Welch Ultimate XB-SiOH (250 mm×50 mm 10 μm) and gradient of 25-55% EtOH+MeOH (4:1, neutral) in n-hexane over 16 min at a flow rate of 25.0 mL/min) to give the title compound (20.0 mg, 46.7 μmol, 33.9% yield, 95.9% purity in HPLC at 220 nm) as a yellow solid. ¹H NMR: (400 MHz, DMSO-d₆) δ 10.9 (br, 1H), 7.92 (s, 1H), 7.84 (d, J=8.0 Hz, 1H), 7.77 (s, 1H), 7.68 (d, J=8.0 Hz, 1H), 5.74 (s, 1H), 5.11 (dd, J=13.2, 5.2 Hz, 1H), 4.40 (dd, J=54.0, 17.6 Hz, 2H), 4.16 (dd, J=11.2, 4.8 Hz, 2H), 3.95 (t, J=10.8 Hz, 2H), 3.80 (s, 3H), 2.97-2.85 (m, 1H), 2.64-2.56 (m, 1H), 2.44-2.31 (m, 1H), 2.15-2.05 (m, 1H), 2.04-1.96 (m, 1H), 1.52-1.45 (m, 1H). (ESI⁺) m/z: 411.1 (M+H)⁺, (C₂₁H₂₂N₄O₅).

### Example 416

### Synthesis of 3-(5-(5-(4-(difluoromethoxy)phenyl)-1-methyl-1H-pyrazol-4-yl)-1-oxoisoindolin-2-yl)piperidine-2,6-dione

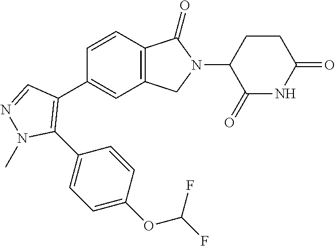 [SMILES: Cn1ncc(-c2ccc3c(c2)CN(C2CCC(=O)NC2=O)C3=O)c1-c1ccc(OC(F)F)cc1]

A. tert-Butyl 5-amino-4-(5-(5-(4-(difluoromethoxy)phenyl)-1-methyl-1H-pyrazol-4-yl)-1-oxoisoindolin-2-yl)-5-oxopentanoate: To a solution of tert-butyl 5-amino-4-[5-(5-bromo-1-methyl-pyrazol-4-yl)-1-oxo-isoindolin-2-yl]-5-oxo-pentanoate (200 mg, 418 μmol, 1.00 eq) and [4-(difluoromethoxy)phenyl]boronic acid (94.4 mg, 502 μmol, 1.20 eq) in dioxane (2.00 mL) and H₂O (0.50 mL) was added Pd(dppf)Cl₂ (34.2 mg, 41.9 μmol, 0.10 eq) and K₃PO₄ (266 mg, 1.26 mmol, 3.00 eq). The mixture was stirred at 60° C. for 16 h under N₂. Then the reaction mixture was filtered and the filtrate was concentrated in vacuum to get a residue. The residue was purified by preparative-TLC (SiO₂, DCM:Methanol=10:1, R_{f}=0.70) to give the title compound (160 mg, 224 μmol, 53.6% yield, 75.9% purity at 220 nm) as yellow oil. (ESI⁺) m/z: 541.1 (M+H)⁺, (C₂₈H₃₀F₂N₄O₅).

B. 3-(5-(5-(4-(Difluoromethoxy)phenyl)-1-methyl-1H-pyrazol-4-yl)-1-oxoisoindolin-2-yl)piperidine-2,6-dione: To a solution of tert-butyl 5-amino-4-[5-[5-[4-(difluoromethoxy)phenyl]-1-methyl-pyrazol-4-yl]-1-oxo-isoindolin-2-yl]-5-oxo-pentanoate (160 mg, 224 μmol, 1.00 eq) in ACN (2.00 mL) was added TsOH (386 mg, 2.25 mmol, 10.0 eq). The mixture was stirred at 80° C. for 2 h. Then the reaction mixture was filtered and the filtrate was concentrated in vacuum to get a residue. The residue was purified by preparative-TLC (SiO₂, DCM:Methanol=10:1, R_{f}=0.60) and then by preparative-HPLC (using a Welch Xtimate C18 (150 mm×25 mm 5 μm) and gradient of 26-56% acetonitrile in water containing 0.5% TFA over 3 min at a flow rate of 25.0 mL/min) to give the title compound (40.0 mg, 83.8 μmol, 32.0% yield, 97.8% purity in HPLC at 220 nm) as a yellow solid. ¹H NMR: (400 MHz, DMSO-d₆) δ 10.9 (s, 1H), 7.89 (s, 1H), 7.56 (d, J=8.0 Hz, 1H), 7.48-7.44 (m, 2H), 7.39 (s, 1H), 7.36 (t, J=74.0 Hz, 1H), 7.33-7.28 (m, 2H), 7.22 (dd, J=8.0, 1.2 Hz, 1H), 5.07 (dd, J=13.2, 5.2 Hz, 1H), 4.28 (dd, J=56.0, 17.2 Hz, 2H), 3.71 (s, 3H), 2.95-2.84 (m, 1H), 2.62-2.58 (m, 1H), 2.43-2.34 (m, 1H), 2.00-1.94 (m, 1H). (ESI⁺) m/z: 466.2 (M+H)⁺, (C₂₄H₂₀F₂N₄O₄).

### Example 417

### Synthesis of 3-(5-(1-(difluoromethyl)-5-(3-fluoro-4-methoxyphenyl)-1H-pyrazol-4-yl)-1-oxoisoindolin-2-yl)piperidine-2,6-dione

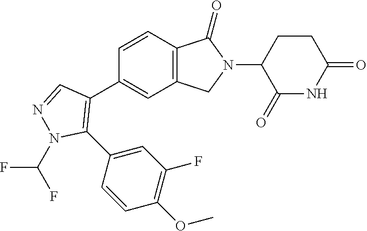 [SMILES: COc1ccc(-c2c(-c3ccc4c(c3)CN(C3CCC(=O)NC3=O)C4=O)cnn2C(F)F)cc1F]

A. 4-Bromo-1-(difluoromethyl)-5-(3-fluoro-4-methoxyphenyl)-1H-pyrazole: To a solution of 4-bromo-1-(difluoromethyl)-1H-pyrazole (200 mg, 1.02 mmol, 1.00 eq), 4-bromo-2-fluoro-1-methoxybenzene (1.25 g, 6.09 mmol, 6.00 eq) and K₂CO₃ (281 mg, 2.03 mmol, 2.00 eq) in DMF (3.00 mL) was added P(oxole)₃, (47.2 mg, 203 μmol, 0.20 eq) and Pd(OAc)₂ (68.4 mg, 304 μmol, 0.30 eq) under N₂. The mixture was stirred at 100° C. for 48 h under N₂. The mixture was poured into H₂O (40.0 mL) and extracted with Ethyl acetate (3×40.0 mL). The combined organic layer was washed with saturated NaCl aqueous (2×50.0 mL), dried over Na₂SO₄ and concentrated under reduced pressure to get residue. The residue was purified by column chromatography (SiO₂, Petroleum ether:Ethyl acetate=10:1, R_{f}=0.50) to get title compound (170 mg, 481 μmol, 47.4% yield, 91.0% purity in LCMS at 220 nm) as light yellow oil. ¹H NMR: (400 MHz, DMSO-d₆) δ 8.05 (s, 1H), 7.65-7.39 (s, 1H), 7.38-7.34 (m, 2H), 7.26-7.24 (m, 1H), 3.92 (s, 3H). ESI⁺ m/z: 320.7 (M+H)⁺, (C₁₁H₈BrF₃N₂O).

B. 3-(5-(1-(Difluoromethyl)-5-(3-fluoro-4-methoxyphenyl)-1H-pyrazol-4-yl)-1-oxoisoindolin-2-yl)piperidine-2,6-dione: To solution of 4-4-bromo-1-(difluoromethyl)-5-(3-fluoro-4-methoxyphenyl)-1H-pyrazole (150 mg, 467 μmol, 1.00 eq), 3-(1-oxo-5-(4,4,5,5-tetramethyl-1,3,2-dioxaborolan-2-yl)isoindolin-2-yl)piperidine-2,6-dione (346 mg, 934 μmol, 2.00 eq) and K₃PO₄ (198 mg, 934 μmol, 2.00 eq) in dioxane (5.00 mL) and H₂O (0.25 mL) was added Ru-Phos-Pd-G₃ (78.1 mg, 93.4 μmol, 0.20 eq) under N₂. The mixture was stirred at 100° C. for 2 h under N₂. The mixture was filtered to collect liquid and concentrated under reduced pressure to get residue. The crude product was purified by preparative-HPLC (using a Phenomenex luna C18 (150 mm×25 mm 10 μm) and gradient of 30-60% acetonitrile in water containing 0.5% TFA over 15 min at a flow rate of 25 mL/min) to give the title compound (168 mg, 344 μmol, 73.8% yield, 99.5% purity in HPLC at 220 nm) as white solid. ¹H NMR: (400 MHz, DMSO-d₆) δ 10.9 (s, 1H), 8.25 (s, 1H), 7.72-7.49 (m, 3H), 7.37-7.30 (m, 3H), 7.29-7.17 (m, 1H), 5.13-5.03 (m, 1H), 4.41-4.22 (m, 2H), 3.90 (s, 3H), 2.93-2.87 (m, 1H), 2.60-2.55 (m, 1H), 2.40-2.35 (m, 1H), 1.99-1.96 (m, 1H). (ESI⁺) m/z: 485.0 (M+H)⁺, (C₂₄H₁₉F₃N₄O₄).

### Example 418

### Synthesis of 3-(5-(1-(difluoromethyl)-5-(4-methoxyphenyl)-1H-pyrazol-4-yl)-1-oxoisoindolin-2-yl)piperidine-2,6-dione

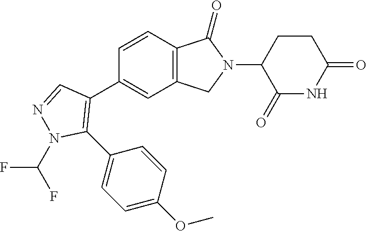 [SMILES: COc1ccc(-c2c(-c3ccc4c(c3)CN(C3CCC(=O)NC3=O)C4=O)cnn2C(F)F)cc1]

A. 4-Bromo-1-(difluoromethyl)-5-(4-methoxyphenyl)-1H-pyrazole: To a solution of 4-bromo-1-(difluoromethyl)-1H-pyrazole (300 mg, 1.52 mmol, 1.00 eq), 1-bromo-4-methoxybenzene (1.71 g, 9.14 mmol, 1.14 mL, 6.00 eq) and K₂CO₃ (421 mg, 3.05 mmol, 2.00 eq) in DMF (4.50 mL) was added P(oxole)₃ (70.7 mg, 304 μmol, 0.20 eq) and Pd(OAc)₂ (68.4 mg, 304 μmol, 0.20 eq) under N₂. The mixture was stirred at 100° C. for 48 h under N₂. The mixture was poured into H₂O (40.0 mL) and extracted with Ethyl acetate (3×40.0 mL). The combined organic layer was washed with saturated NaCl aqueous (2×50.0 mL), dried over Na₂SO₄ and concentrated under reduced pressure to get residue. The residue was purified by column chromatography (SiO₂, Petroleum ether:Ethyl acetate=10:1, R_{f}=0.50) to get the title compound (180 mg, 579. μmol, 38.0% yield, 97.5% purity) as colorless oil. ¹H NMR: (400 MHz, DMSO-d₆) δ 8.03 (s, 1H), 7.75-7.47 (m, 1H), 7.40-7.37 (m, 2H), 7.14-7.11 (m, 2H), 3.83 (s, 3H). (ESI⁺) m/z: 303.0 (M+H)⁺, (C₁₁H₉BrF₂N₂O).

B. 3-(5-(1-(Difluoromethyl)-5-(4-methoxyphenyl)-1H-pyrazol-4-yl)-1-oxoisoindolin-2-yl)piperidine-2,6-dione: To a solution of 4-bromo-1-(difluoromethyl)-5-(4-methoxyphenyl)-1H-pyrazole (180 mg, 594 μmol, 1.00 eq), 3-(1-oxo-5-(4,4,5,5-tetramethyl-1,3,2-dioxaborolan-2-yl)isoindolin-2-yl)piperidine-2,6-dione (439 mg, 1.19 mmol, 2.00 eq) and K₃PO₄ (252 mg, 1.19 mmol, 2.00 eq) in dioxane (5.00 mL) and H₂O (0.25 mL) was added Ru-Phos-Pd-G₃, (99.3 mg, 118 μmol, 0.20 eq) under N₂. The mixture was stirred at 100° C. for 2 h under N₂. The mixture was filtered to collect liquid and concentrated under reduced pressure to get residue. The crude product was purified by preparative-HPLC (using a Phenomenex luna C18 (150 mm×25 mm×10 μm) and gradient of 25-55% acetonitrile in water containing 0.5% TFA over 15 min at a flow rate of 25 mL/min) to give the title compound (206 mg, 440 μmol, 74.2% yield, 99.8% purity in HPLC at 220 nm) as white solid. ¹H NMR: (400 MHz, DMSO-d₆) δ 10.9 (s, 1H), 8.24 (s, 1H), 7.69-7.42 (m, 3H), 7.33-7.27 (m, 3H), 7.09-7.07 (m, 2H), 5.10-5.06 (m, 1H), 4.40-4.21 (m, 2H), 3.82 (s, 3H), 2.91-2.86 (m, 1H), 2.60-2.55 (m, 1H), 2.40-2.35 (m, 1H), 1.99-1.97 (m, 1H). (ESI⁺) m/z: 467.0 (M+H)⁺, (C₂₄H₂₀F₂N₄O₄).

### Example 419

### Synthesis of 3-(5-(2-(1,3-dioxan-2-yl)-1-methyl-5-phenyl-1H-imidazol-4-yl)-1-oxoisoindolin-2-yl)piperidine-2,6-dione

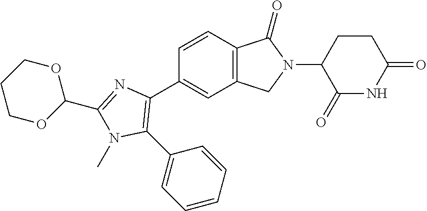 [SMILES: Cn1c(C2OCCCO2)nc(-c2ccc3c(c2)CN(C2CCC(=O)NC2=O)C3=O)c1-c1ccccc1]

A. 1-Methyl-5-phenyl-1H-imidazole: A mixture of benzaldehyde (4.50 g, 42.4 mmol, 4.29 mL, 1.00 eq), DIEA (10.9 g, 84.8 mmol, 14.7 mL, 2.00 eq) and MeNH₂·HCl (5.73 g, 84.8 mmol, 2.00 eq) in DMF (45.0 mL) was stirred at 25° C. for 2 h under N₂ atmosphere. Then K₂CO₃ (8.79 g, 63.6 mmol, 1.50 eq) and TosMIC (9.93 g, 50.8 mmol, 1.20 eq) was added and the reaction mixture was stirred at 50° C. for 32 h under N₂ atmosphere. Then the reaction mixture was filtered and the filtrate was concentrated in vacuum to get a residue. The residue was purified by column chromatography (SiO₂, DCM:Methanol=100:1 to 10:1, R_{f}=0.60 (DCM:Methanol=10:1)) to give the title compound (4.00 g, 23.6 mmol, 55.8% yield, 93.7% purity in LCMS at 220 nm) as a brown solid. ¹H NMR: (400 MHz, CDCl₃) δ 7.52 (s, 1H), 7.47-7.35 (m, 5H), 7.11 (s, 1H), 3.67 (s, 3H). (ESI⁺) m/z: 159.0 (M+H)⁺, (C₁₀H₁₀N₂).

B. 1-Methyl-5-phenyl-1H-imidazole-2-carbaldehyde: To a solution of 1-methyl-5-phenyl-imidazole (2.00 g, 11.8 mmol, 1.00 eq) in THF (25.0 mL) was added n-BuLi (2.50 M, 7.11 mL, 1.50 eq) at −78° C. under N₂. The reaction mixture was stirred at −78° C. for 2 h. Then DMF (1.73 g, 23.6 mmol, 1.82 mL, 2.00 eq) was added dropwise and the reaction mixture was warmed to 25° C. and stirred for 24 h under N₂. Then the reaction mixture was quenched with 20.0 mL of saturated NH₄Cl solution and extracted with EtOAc (3×20.0 mL). The combined organic layers were washed with brine (20.0 mL), dried over Na₂SO₄ and filtered. The filtrate was concentrated in vacuum to get a residue. The residue was purified by column chromatography (SiO₂, Petroleum ether:Ethyl acetate=100:1 to 10:1, R_{f}=0.50 (Petroleum ether:Ethyl acetate=1:1)) to give the title compound (1.20 g, 6.34 mmol, 53.5% yield, 98.4% purity in LCMS at 220 nm) as a yellow solid. (ESI⁺) m/z: 187.0 (M+H)⁺, (C₁₁H₁₀N₂O).

C. 2-(1,3-Dioxan-2-yl)-1-methyl-5-phenyl-1H-imidazole: To a solution of 1-methyl-5-phenyl-imidazole-2-carbaldehyde (600 mg, 3.17 mmol, 1.00 eq) and propane-1,3-diol (482 mg, 6.34 mmol, 458 μL, 2.00 eq) in toluene (18.0 mL) was added TsOH (163 mg, 951 μmol, 0.30 eq). The mixture was stirred at 110° C. in a Dean-Stark trap apparatus for 12 h. Then the reaction mixture was concentrated in vacuum to get a residue. The residue was diluted with 10.0 mL of EtOAc and washed with water (2×10.0 mL) and brine (10.0 mL). The organic layer was concentrated in vacuum to give the title compound (600 mg, 2.42 mmol, 76.3% yield, 98.6% purity in LCMS at 220 nm) as a brown solid. ¹H NMR: (400 MHz, DMSO-d₆) δ 7.49-7.44 (m, 4H), 7.43-7.38 (m, 1H), 6.94 (s, 1H), 5.71 (s, 1H), 4.19-4.13 (m, 2H), 4.00-3.92 (m, 2H), 3.74 (s, 3H), 2.14-2.01 (m, 1H), 1.52-1.44 (m, 1H). (ESI⁺) m/z: 245.1 (M+H)⁺, (C₁₄H₁₆N₂O₂).

D. 4-Bromo-2-(1,3-dioxan-2-yl)-1-methyl-5-phenyl-1H-imidazole: To a solution of 2-(1,3-dioxan-2-yl)-1-methyl-5-phenyl-imidazole (200 mg, 807 μmol, 1.00 eq) in ACN (2.00 mL) was added NBS (158 mg, 888 μmol, 1.10 eq). The mixture was stirred at 25° C. for 2 h. Then the reaction mixture was concentrated in vacuum to get a residue. The residue was diluted with 10.0 mL of EtOAc and washed with water (2×10.0 mL) and brine (10.0 mL).

The organic layer was concentrated in vacuum to give the title compound (250 mg, 736 μmol, 91.1% yield, 95.1% purity in LCMS at 220 nm) as a brown solid. ¹H NMR: (400 MHz, DMSO-d₆) δ 7.55-7.43 (m, 5H), 5.69 (s, 1H), 4.20-4.13 (m, 2H), 4.00-3.92 (m, 2H), 3.64 (s, 3H), 2.11-2.01 (m, 1H), 1.52-1.45 (m, 1H). (ESI⁺) m/z: 323.0 (M+H)⁺, (C₁₄H₁₅BrN₂O₂).

E. 3-(5-(2-(1,3-Dioxan-2-yl)-1-methyl-5-phenyl-1H-imidazol-4-yl)-1-oxoisoindolin-2-yl)piperidine-2,6-dione: To a solution of 4-bromo-2-(1,3-dioxan-2-yl)-1-methyl-5-phenyl-imidazole (231 mg, 680 μmol, 1.00 eq) and 3-[1-oxo-5-(4,4,5,5-tetramethyl-1,3,2-dioxaborolan-2-yl)isoindolin-2-yl]piperidine-2,6-dione (504 mg, 1.36 mmol, 2.00 eq) in dioxane (5.00 mL) and H₂O (0.25 mL) was added Ru-Phos-Pd-G₃ (56.9 mg, 68.0 μmol, 0.10 eq) and K₃PO₄ (289 mg, 1.36 mmol, 2.00 eq). The mixture was stirred at 100° C. for 2 h under N₂. Then the reaction mixture was filtered and the filtrate was concentrated in vacuum to get a residue. The residue was purified by preparative-HPLC (using a Welch Ultimate XB-SiOH (250 mm×50 mm 10 μm) and gradient of 20-50% EtOH+MeOH (4:1, neutral) in n-hexane over 16 min at a flow rate of 25.0 mL/min) to give the title compound (91.9 mg, 181 μmol, 26.7% yield, 96.3% purity in HPLC at 220 nm) as a white solid. ¹H NMR: (400 MHz, DMSO-d₆) δ 10.9 (br, 1H), 7.58 (s, 1H), 7.56-7.49 (m, 4H), 7.43-7.35 (m, 3H), 5.82 (s, 1H), 5.06 (dd, J=13.2, 5.2 Hz, 1H), 4.34 (d, J=17.2 Hz, 1H), 4.23-4.16 (m, 3H), 4.04-3.95 (m, 2H), 3.56 (s, 3H), 2.94-2.84 (m, 1H), 2.60-2.53 (m, 1H), 2.41-2.29 (m, 1H), 2.15-2.04 (m, 1H), 2.00-1.92 (m, 1H), 1.54-1.46 (m, 1H). (ESI⁺) m/z: 487.2 (M+H)⁺, (C₂₇H₂₆N₄O₅).

### Example 420

### Synthesis of 3-(5-(1-methyl-5-(pyridin-3-yl)-2-(tetrahydro-2H-pyran-4-yl)-1H-imidazol-4-yl)-1-oxoisoindolin-2-yl)piperidine-2,6-dione

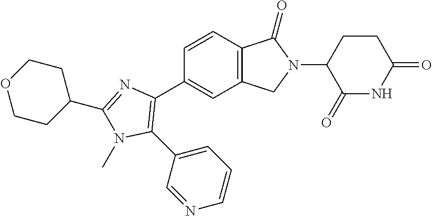 [SMILES: Cn1c(C2CCOCC2)nc(-c2ccc3c(c2)CN(C2CCC(=O)NC2=O)C3=O)c1-c1cccnc1]

A. 5-Bromo-1-methyl-2-(tetrahydro-2H-pyran-4-yl)-1H-imidazole: To a solution of 1-methyl-2-tetrahydropyran-4-yl-imidazole (1.00 g, 6.02 mmol, 1.00 eq) in ACN (5.00 mL) was added the solution of NBS (1.12 g, 6.32 mmol, 1.05 eq) in ACN (5.00 mL) at 0° C. The reaction mixture was stirred at 25° C. for 4 h. The reaction mixture was poured into H₂O (20.0 mL), extracted with EtOAc (3×15.0 mL), dried over Na₂SO₄ and concentrated in vacuum to get residue. The crude product was purified by column chromatography (SiO₂, Methanol:Dichloromethane=100:1 to 20:1; TLC, Methanol:Dichloromethane=20:1, R_{f}=0.50) to give the title compound (1.00 g, 4.08 mmol, 67.8% yield). ¹H NMR: (400 MHz, CDCl₃) δ 6.95 (s, 1H), 4.10-4.07 (m, 2H), 3.56 (s, 3H), 3.53-3.48 (m, 2H), 2.94-2.87 (m, 1H), 2.04-1.96 (m, 2H), 1.80-1.76 (m, 2H). (ESI⁺) m/z: 246.1 (M+H)⁺, (C₉H₁₃BrN₂O).

B. 3-(1-Methyl-2-(tetrahydro-2H-pyran-4-yl)-1H-imidazol-5-yl)pyridine: To a solution of 5-bromo-1-methyl-2-tetrahydropyran-4-yl-imidazole (0.10 g, 407 μmol, 1.00 eq) and 3-pyridylboronic acid (100 mg, 815 μmol, 2.00 eq) in dioxane (1.00 mL) and H₂O (0.10 mL) was added K₃PO₄ (173 mg, 815 μmol, 2.00 eq) and Pd(PPh₃)₄ (47.1 mg, 40.8 μmol, 0.10 eq) under N₂. The reaction mixture was stirred at 90° C. for 12 h under N₂. The residue was purified by preparative-TLC (SiO₂, Dichloromethane:Methanol=20:1; TLC, Dichloromethane:Methanol=20:1, R_{f}=0.20) to give the title compound (0.22 g, 904 μmol, 55.4% yield). (ESI⁺) m/z: 244.1 (M+H)⁺, (C₁₄H₁₇N₃O).

C. 3-(4-Bromo-1-methyl-2-(tetrahydro-2H-pyran-4-yl)-1H-imidazol-5-yl)pyridine: To a solution of 3-(3-methyl-2-tetrahydropyran-4-yl-imidazol-4-yl)pyridine (0.22 g, 745 μmol, 1.00 eq) in DMF (2.00 mL) was added a solution of NBS (159 mg, 894 μmol, 1.20 eq) in DMF (2.00 mL) at 0° C. The reaction mixture was stirred at 25° C. for 10 h. The residue was purified by preparative-TLC (SiO₂, Dichloromethane:Methanol=20:1; TLC, Dichloromethane:Methanol=20:1, R_{f}=0.30) to give the title compound (0.20 g, 611 μmol, 82.0% yield). ¹H NMR: (400 MHz, CDCl₃) δ 8.64-8.61 (m, 2H), 7.90-7.87 (m, 1H), 7.55-7.52 (m, 1H), 3.94-3.90 (m, 2H), 3.51 (s, 3H), 3.49-3.45 (m, 2H), 3.32-3.31 (m, 1H), 1.78-1.73 (m, 4H). (ESI⁺) m/z: 323.8 (M+H)⁺, (C₁₄H₁₆BrN₃O).

D. 3-(5-(1-Methyl-5-(pyridin-3-yl)-2-(tetrahydro-2H-pyran-4-yl)-1H-imidazol-4-yl)-1-oxoisoindolin-2-yl)piperidine-2,6-dione: To a solution of 3-(5-bromo-3-methyl-2-tetrahydropyran-4-yl-imidazol-4-yl)pyridine (150 mg, 465 μmol, 1.00 eq), 3-[1-oxo-5-(4,4,5,5-tetramethyl-1,3,2-dioxaborolan-2-yl)isoindolin-2-yl]piperidine-2,6-dione (344 mg, 931 μmol, 2.00 eq) in dioxane (3.00 mL) and H₂O (0.15 mL) was added Ru-Phos-Pd-G₃ (38.9 mg, 46.5 μmol, 0.10 eq), K₃PO₄ (197 mg, 931 μmol, 2.00 eq) under N₂. The reaction mixture was stirred at 100° C. for 2 h under N₂. The reaction mixture was concentrated in vacuum to give residue. The residue was purified by preparative-HPLC (using a Welch Xtimate C18 (150 mm×25 mm 5 μm) and gradient of 2-32% acetonitrile in water containing 0.05% TFA over 10 min at a flow rate of 25.0 mL/min) to give the title compound (82.9 mg, 169 μmol, 36.5% yield, 99.5% purity in HPLC at 220 nm) as white solid. ¹H NMR: (400 MHz, MeOD) δ 8.76-8.74 (m, 1H), 8.61 (s, 1H), 8.02-8.00 (m, 1H), 7.83-7.81 (m, 1H), 7.66-7.65 (m, 1H), 7.64 (s, 1H), 7.59-7.50 (m, 1H), 5.17-5.13 (m, 1H), 4.48-4.41 (m, 2H), 4.14-4.11 (m, 2H), 3.76 (s, 3H), 3.70-3.64 (m, 3H), 2.87-2.80 (m, 1H), 2.79-2.78 (m, 1H), 2.14-2.10 (m, 1H), 2.08-2.04 (m, 5H). (ESI⁺) m/z: 486.3 (M+H)⁺, (C₂₇H₂₇N₅O₄).

### Example 421

### Synthesis of 3-(5-(1-(difluoromethyl)-2-phenyl-1H-imidazol-4-yl)-1-oxoisoindolin-2-yl)piperidine-2,6-dione

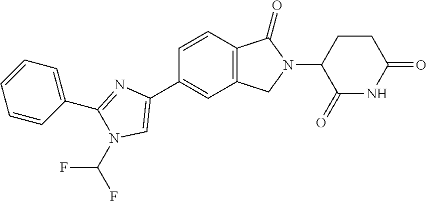 [SMILES: O=C1CCC(N2Cc3cc(-c4cn(C(F)F)c(-c5ccccc5)n4)ccc3C2=O)C(=O)N1]

A. 1-(Difluoromethyl)-2-phenyl-1H-imidazole: To a solution of 2-phenyl-1H-imidazole (5.00 g, 34.6 mmol, 1.00 eq) and 1-[[bromo(difluoro)methyl]-ethoxy-phosphoryl]oxyethane (9.26 g, 34.6 mmol, 1.00 eq) in ACN (100 mL) was added KF (4.03 g, 69.3 mmol, 1.62 mL, 2.00 eq) under N₂. The reaction mixture was stirred at 25° C. for 12 h under N₂. The reaction mixture was concentrated in vacuum to give residue. The residue was dissolved in ethyl acetate (100 mL), washed with H₂O (2×50.0 mL), dried over Na₂SO₄ and concentrated in vacuum to give residue. The residue was purified by column chromatography (SiO₂, Petroleum ether:Ethyl acetate=100:1 to 3:1; TLC, Petroleum ether:Ethyl acetate=3:1, R_{f}=0.30) to give the title compound (6.50 g, 24.1 mmol, 69.5% yield, 72% purity in HPLC at 220 nm) as colorless oil. (ESI⁺) m/z: 195.1 (M+H)⁺, (C₁₀H₈F₂N₂).

B. 5-Bromo-1-(difluoromethyl)-2-phenyl-1H-imidazole: A solution of 1-(difluoromethyl)-2-phenyl-imidazole (1.00 g, 3.71 mmol, 1.00 eq) and NBS (1.98 g, 11.1 mmol, 3.00 eq) in ACN (20.0 mL) was stirred at 50° C. for 12 h. The reaction mixture was poured into H₂O (15.0 mL), extracted with ethyl acetate (3×15.0 mL), dried over Na₂SO₄ and concentrated in vacuum to give residue. The residue was purified by column chromatography (SiO₂, Petroleum ether:Ethyl acetate=100:1 to 3:1; TLC, Petroleum ether: Ethyl acetate=3:1, R_{f}=0.40) to give the title compound (0.60 g, 2.20 mmol, 59.2% yield) as yellow solid. (ESI⁺) m/z: 274.0 (M+H)⁺, (C₁₀H₇BrF₂N₂).

C. 4,5-Dibromo-1-(difluoromethyl)-2-phenyl-1H-imidazole: A mixture of 5-bromo-1-(difluoromethyl)-2-phenyl-imidazole (0.50 g, 1.83 mmol, 1.00 eq) and NBS (651 mg, 3.66 mmol, 2.00 eq) in DMF (10.0 mL) was stirred at 25° C. for 2 h. The reaction mixture was poured into H₂O (10.0 mL), extracted with ethyl acetate (3×15.0 mL), dried over Na₂SO₄ and concentrated in vacuum to give residue. The residue was purified by column chromatography (SiO₂, Petroleum ether:Ethyl acetate=100:1 to 3:1; TLC, Petroleum ether: Ethyl acetate=3:1,R_{f}=0.60) to give the title compound (0.30 g, 842 μmol, 46.0% yield, 98.9% purity in HPLC at 220 nm) as colorless oil. (ESI⁺) m/z: 352.1 (M+H)⁺, (C₁₀H₆Br₂F₂N₂).

D. 4-Bromo-1-(difluoromethyl)-2-phenyl-1H-imidazole: To a solution of 4,5-dibromo-1-(difluoromethyl)-2-phenyl-imidazole (0.20 g, 568 μmol, 1.00 eq) in THF (4.00 mL) was added EtMgBr (3.00 M, 625 μL, 3.30 eq) dropwise at 0° C. under N₂. The reaction mixture was stirred at 0° C. for 2 h under N₂. The reaction mixture was quenched with saturated NaHCO₃ solution (3.00 mL) and H₂O (3.00 mL). The aqueous phase was extracted with ethyl acetate (3×10.0 mL), dried over Na₂SO₄ and concentrated in vacuum to give a residue. The residue was purified by preparative-TLC (SiO₂, Dichloromethane:Methanol=20:1; TLC, Dichloromethane:Methanol=20:1, R_{f}=0.40) to give the title compound (0.30 g, 842 μmol, 64.4% yield, 98.9% purity in HPLC at 220 nm) as colorless oil. (ESI⁺) m/z: 272.9 (M+H)⁺, (C₁₀H₇Br₂F₂N₂).

E. 3-(5-(1-(Difluoromethyl)-2-phenyl-1H-imidazol-4-yl)-1-oxoisoindolin-2-yl)piperidine-2,6-dione: To a solution of 4-bromo-1-(difluoromethyl)-2-phenyl-imidazole (0.10 g, 366 μmol, 1.00 eq), 3-[1-oxo-5-(4,4,5,5-tetramethyl-1,3,2-dioxaborolan-2-yl)isoindolin-2-yl]piperidine-2,6-dione (271 mg, 732 μmol, 2.00 eq) in dioxane (2.00 mL) and H₂O (0.10 mL) was added K₃PO₄ (155 mg, 732 μmol, 2.00 eq) and Ru-Phos-Pd-G₃ (30.6 mg, 36.6 μmol, 0.10 eq) under N₂. The reaction mixture was stirred at 100° C. for 2 h under N₂. The reaction mixture was concentrated in vacuum to give residue. The residue was purified by preparative-HPLC (using a Phenomenex Luna (200 mm×40 mm×10 m) and gradient of 27-57% acetonitrile in water containing 0.05% TFA over 10 min at a flow rate of 25 mL/min) to give the title compound (61.8 mg, 141 μmol, 38.4% yield, 99.8% purity in HPLC at 220 nm) as white solid. ¹H NMR: (400 MHz, DMSO-d₆) δ 11.0 (s, 1H), 8.46 (s, 1H), 8.16 (s, 1H), 8.08 (d, J=8.4 Hz, 1H), 7.93-7.64 (m, 4H), 7.61-7.58 (m, 3H), 5.15-5.11 (m, 1H), 4.53-4.36 (m, 2H), 2.97-2.88 (m, 1H), 2.63-2.58 (m, 1H), 2.44-2.37 (m, 1H), 2.04-1.99 (m, 1H). (ESI⁺) m/z: 437.2 (M+H)⁺, (C₂₃H₁₈F₂N₄O₃).

### Example 422

### Synthesis of 3-(5-(1-(difluoromethyl)-5-phenyl-1H-pyrazol-4-yl)-1-oxoisoindolin-2-yl)piperidine-2,6-dione

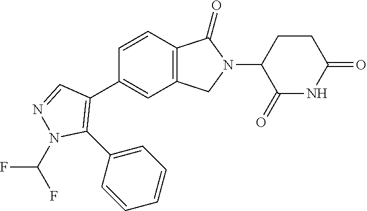 [SMILES: O=C1CCC(N2Cc3cc(-c4cnn(C(F)F)c4-c4ccccc4)ccc3C2=O)C(=O)N1]

A. 4-Bromo-1-(difluoromethyl)-5-phenyl-1H-pyrazole: To a solution of 4-bromo-1-(difluoromethyl)pyrazole (200 mg, 1.02 mmol, 1.00 eq) in DMF (4.00 mL) was added bromobenzene (956 mg, 6.09 mmol, 641 μL, 6.00 eq), P(oxole)₃ (47.1 mg, 203 μmol, 0.20 eq), K₂CO₃ (280 mg, 2.03 mmol, 2.00 eq) and Pd(OAc)₂ (68.3 mg, 304 μmol, 0.30 eq) under N₂. The mixture was stirred at 100° C. for 48 h under N₂. After the reaction was completed, the reaction mixture was cooled to 25° C. and filtered. The filtrate was poured into H₂O (20.0 mL) and extracted with ethyl acetate (3×20.0 mL). The organic layer was washed with brine (30.0 mL), dried over Na₂SO₄, filtered and the filtrate was concentrated in vacuum to get a residue. The residue was purified by column chromatography (SiO₂, Petroleum ether:Ethyl acetate=100:1 to 10:1, R_{f}=0.50 (Petroleum ether:Ethyl acetate=10:1)) and by preparative-TLC (Dichloromethane: Petroleum ether=2:1, R_{f}=0.60) to give the title compound (120 mg, 413 μmol, 40.6% yield, 94.0% purity in LCMS at 220 nm) as yellow oil. ¹H NMR: (400 MHz, DMSO-d₆) δ 8.06 (s, 1H), 7.65 (t, J=57.2 Hz, 1H), 7.57 (t, J=3.6 Hz, 3H), 7.46 (dd, J=6.8, 3.2 Hz, 2H). (ESI⁺) m/z: 272.9 (M+H)⁺, (C₁₀H₇BrF₂N₂).

B. 3-(5-(1-(Difluoromethyl)-5-phenyl-1H-pyrazol-4-yl)-1-oxoisoindolin-2-yl)piperidine-2,6-dione: To a solution of 4-bromo-1-(difluoromethyl)-5-phenyl-pyrazole (110 mg, 378 μmol, 1.00 eq) and 3-[1-oxo-5-(4,4,5,5-tetramethyl-1,3,2-dioxaborolan-2-yl)isoindolin-2-yl]piperidine-2,6-dione (350 mg, 946 μmol, 2.50 eq) in dioxane (2.00 mL) and H₂O (0.10 mL) was added Ru-Phos-Pd-G₃ (31.6 mg, 37.8 μmol, 0.10 eq) and K₃PO₄ (160 mg, 757 μmol, 2.00 eq) under N₂. The mixture was stirred at 100° C. for 2 h under N₂. After the reaction was completed, the reaction mixture was filtered and the filtrate was concentrated in vacuum to give a residue. The residue was purified by preparative-TLC (Dichloromethane:Methanol=10:1, R_{f}=0.50) and then by preparative-HPLC (using a Welch Xtimate C18 (150 mm×25 mm×5 μm) and gradient of 27-57% acetonitrile in water containing 0.5% TFA over 10 min at a flow rate of 25.0 mL/min) to give the title compound (122 mg, 272 μmol, 71.8% yield, 96.9% purity in HPLC at 220 nm) as a white solid. ¹H NMR: (400 MHz, DMSO-d₆) δ 10.9 (s, 1H), 8.2 (s, 1H), 7.60-7.53 (m, 5H), 7.46-7.39 (m, 3H), 7.26 (d, J=9.2 Hz, 1H), 5.07 (dd, J=13.2, 5.2 Hz, 1H), 4.29 (dd, J=58.0, 17.6 Hz, 2H), 2.91-2.85 (m, 1H), 2.60-2.55 (m, 1H), 2.40-2.32 (m, 1H), 1.99-1.96 (m, 1H). (ESI⁺) m/z: 437.1 (M+H)⁺, (C₂₃H₁₈F₂N₄O₃).

### Example 423

### Synthesis of 3-(5-(1-(difluoromethyl)-3-(2,3-dihydrobenzofuran-5-yl)-1H-pyrazole-4-yl)-1-oxoisoindolin-2-yl)piperidine-2,6-dione

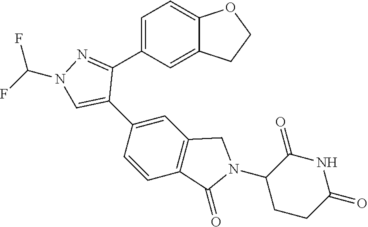 [SMILES: O=C1CCC(N2Cc3cc(-c4cn(C(F)F)nc4-c4ccc5c(c4)CCO5)ccc3C2=O)C(=O)N1]

A. 5-(2,3-Dihydrobenzofuran-5-yl)-1H-pyrazole: To a solution of 5-bromo-1H-pyrazole (1.00 g, 6.80 mmol, 1.00 eq), (2,3-dihydrobenzofuran-5-yl)boronic acid (1.34 g, 8.16 mmol, 1.20 eq) and K₃PO₄ (2.89 g, 13.6 mmol, 2.00 eq) in dioxane (10.0 mL) and H₂O (1.00 mL) was added PdCl₂(dtbpf) (443 mg, 680 μmol, 0.10 eq) under N₂. The mixture was stirred at 90° C. for 12 h under N₂. The reaction mixture was poured into H₂O (50 mL) and extracted with Ethyl acetate (3×80 mL). The combined organic layer was dried over Na₂SO₄, filtered and concentrated. The residue was purified by column chromatography (SiO₂, Petroleum ether:Ethyl acetate=40:1 to 1:1, TLC:Petroleum ether:Ethyl acetate=1: 1, R_{f}=0.20) to give the title compound (478 mg, 2.53 mmol, 37.2% yield, 98.7% purity in LCMS at 220 nm) as a white solid. (ESI⁺) m/z: 187.0 (M+H)⁺, (C₁₁H₁₀N₂O).

B. 4-Bromo-5-(2,3-dihydrobenzofuran-5-yl)-1H-pyrazole: To a solution of 5-(2,3-dihydrobenzofuran-5-yl)-1H-pyrazole (200 mg, 1.07 mmol, 1.00 eq) in DMF (3.00 mL) was added NBS (191 mg, 1.07 mmol, 1.00 eq) at 0° C. The mixture was stirred at 0° C. for 1 h. The reaction mixture was poured into H₂O (50 mL) and extracted with Ethyl acetate (3×50 mL). The combined organic layer was washed with brine (3×50 mL), dried over Na₂SO₄, filtered and concentrated. The residue was purified by preparative-TLC (TLC:Petroleum ether:Ethyl acetate=1:1, R_{f}=0.40) to give the title compound (170 mg, 641 μmol, 59.7% yield) as a yellow oil. ¹HNMR: (400 MHz, DMSO-d₆) δ 8.62 (s, 1H), 7.63-7.58 (m, 2H), 6.95-6.83 (m, 1H), 4.65-4.53 (t, 2H), 3.27-3.22 (t, 2H). (ESI⁺) m/z: 265.1 (M+H)⁺, (C₁₁H₉BrN₂O).

C. 4-Bromo-1-(difluoromethyl)-3-(2,3-dihydrobenzofuran-5-yl)-1H-pyrazole: To a solution of 4-bromo-5-(2,3-dihydrobenzofuran-5-yl)-1H-pyrazole (100 mg, 377 μmol, 1.00 eq) and KF (43.8 mg, 754 μmol, 17.8 μL, 2.00 eq) in ACN (3.00 mL). The mixture was stirred until a colorless oil was formed. Then diethyl (bromodifluoromethyl) phosphonate (110 mg, 415 μmol, 1.10 eq) was added to the mixture at 25° C. under N₂. The mixture was stirred at 25° C. for 12 h under N₂. The reaction mixture was concentrated under reduced pressure to get a residue at 40° C. The residue was purified by preparative-TLC (Petroleum ether:Ethyl acetate=5:1, R_{f}=0.5) to give the title compound (130 mg, 360 μmol, 95.4% yield, 87.3% purity in LCMS at 220 nm) as a light yellow solid. ¹HNMR: (400 MHz, DMSO-d₆) δ 8.62 (s, 1H), 8.02-7.68 (m, 1H), 7.63-7.58 (m, 2H), 6.95-6.83 (m, 1H), 4.65-4.53 (t, 2H), 3.27-3.22 (t, 2H). (ESI⁺) m/z: 316.9 (M+H)⁺, (C₁₂H₉BrF₂N₂O).

D. 3-(5-(1-(difluoromethyl)-3-(2,3-dihydrobenzofuran-5-yl)-1H-pyrazol-4-yl)-1-oxoisoindolin-2-yl)piperidine-2,6-dione: To a solution of 4-bromo-1-(difluoromethyl)-3-(2,3-dihydrobenzofuran-5-yl)-1H-pyrazole (110 mg, 349 μmol, 1.00 eq), 3-(1-oxo-5-(4,4,5,5-tetramethyl-1,3,2-dioxaborolan-2-yl)isoindolin-2-yl)piperidine-2,6-dione (258 mg, 698 μmol, 2.00 eq) and K₃PO₄ (222 mg, 1.05 mmol, 3.00 eq) in dioxane (3.00 mL) and H₂O (0.15 mL) were added Ru-Phos-Pd-G₃ (43.8 mg, 52.4 μmol, 0.15 eq) under N₂. Then the mixture was stirred at 100° C. for 2 h. The mixture was concentrated under vacuum to get residue. The residue was purified by preparative-HPLC (using a Phenomenex luna C18 (150×25 mm×5 μm) and gradient of 21.0%-35.0% acetonitrile in water containing 0.50% TFA over 18 min at a flow rate of 25 mL/min) to give the title compound (18.1 mg, 37.9 μmol, 11.8% yield, 99.0% purity in HPLC at 220 nm) as white solid. ¹H NMR: (400 MHz, DMSO-d₆) δ 11.01 (s, 1H), 8.56 (s, 1H), 8.04-7.88 (m, 2H), 7.58 (s, 1H), 7.40-7.36 (m, 1H), 7.09-7.06 (m, 1H), 7.07 (d, J=8.4 Hz, 1H), 6.75 (d, J=8.4 Hz, 1H), 5.15-5.10 (m, 1H), 4.58-4.54 (t, 2H), 4.43-4.29 (q, 2H), 3.20-3.15 (t, 2H), 2.92-2.89 (m, 1H), 2.63-2.60 (m, 1H), 2.43-2.39 (m, 1H), 2.03-2.00 (m, 1H). (ESI⁺) m/z: 479.2 (M+H)⁺, (C₂₅H₂₀F₂N₄O₄).

### Example 424

### Synthesis of 3-(5-(1-(difluoromethyl)-5-(2,3-dihydrobenzofuran-5-yl)-1H-pyrazol-4-yl)-1-oxoisoindolin-2-yl)piperidine-2,6-dione

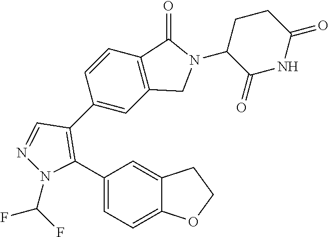 [SMILES: O=C1CCC(N2Cc3cc(-c4cnn(C(F)F)c4-c4ccc5c(c4)CCO5)ccc3C2=O)C(=O)N1]

A. 4-Bromo-1-(difluoromethyl)-5-(2,3-dihydrobenzofuran-5-yl)-1H-pyrazole: To a solution of 4-bromo-1-(difluoromethyl)-1H-pyrazole (300 mg, 1.52 mmol, 1.00 eq) and 5-bromo-2,3-dihydrobenzofuran (1.82 g, 9.14 mmol, 6.00 eq) in DMF (7.00 mL) was added K₂CO₃ (490 mg, 3.05 mmol, 2.00 eq), P(oxole)₃ (70.7 mg, 304 μmol, 0.20 eq) and Pd(OAc)₂ (102 mg, 456 μmol, 0.30 eq) under N₂. The mixture was stirred at 100° C. for 48 h under N₂. The reaction mixture was poured into H₂O (10.0 mL) and extracted with Ethyl acetate (3×30.0 mL). The combined organic layer was washed with brine (3×30.0 mL), and dried over by Na₂SO₄, filtered and concentrate filtrate under reduced pressure to get residue. The residue was purified by preparative-TLC (Petroleum ether:Ethyl acetate=20:1,R_{f}=0.40) to give the title compound (60.0 mg, 193 μmol, 12.5% yield, 98.4% purity in LCMS at 220 nm) as light yellow oil. ¹H NMR: (400 MHz, DMSO-d₆) δ 8.03-8.00 (m, 1H), 7.73-7.42 (m, 1H), 7.30-7.15 (m, 2H), 6.95-6.93 (m, 1H), 4.67-4.60 (m, 2H), 3.28-3.24 (m, 2H). (ESI⁺) m/z: 317.0 (M+H)⁺, (C₁₂H₉BrF₂N₂O).

B. 3-(5-(1-(difluoromethyl)-5-(2,3-dihydrobenzofuran-5-yl)-1H-pyrazol-4-yl)-1-oxoisoindolin-2-yl)piperidine-2,6-dione: To a solution of 4-bromo-1-(difluoromethyl)-5-(2,3-dihydrobenzofuran-5-yl)-1H-pyrazole (60.0 mg, 190 μmol, 1.00 eq), 3-(1-oxo-5-(4,4,5,5-tetramethyl-1,3,2-dioxaborolan-2-yl)isoindolin-2-yl)piperidine-2,6-dione (140 mg, 380 μmol, 2.00 eq) and K₃PO₄ (121 mg, 571 μmol, 3.00 eq) in dioxane (3.00 mL) and H₂O (0.15 mL) was added Ru-Phos-Pd-G₃ (15.9 mg, 19.0 μmol, 0.10 eq) under N₂. The mixture was stirred at 100° C. for 3 h under N₂. The reaction mixture was concentrated under reduced pressure to get a residue at 45° C. The residue was purified by preparative-HPLC (using a Phenomenex luna C18 (150×25 mm×5 μm) and gradient of 28.0%-58.0% acetonitrile in water containing 0.05% FA over 10 min at a flow rate of 25 mL/min) to give the title compound (49.1 mg, 102 μmol, 53.8% yield, 99.8% purity in HPLC at 220 nm) as white solid. ¹H NMR: (400 MHz, DMSO-d₆) δ 10.9 (s, 1H), 8.24 (s, 1H), 7.69-7.51 (m, 3H), 7.40-7.25 (m, 2H), 7.10-7.08 (m, 1H), 6.90-6.88 (m, 1H), 5.11-5.06 (m, 1H), 4.63-4.59 (m, 2H), 4.41-4.22 (m, 2H), 3.26-3.20 (m, 2H), 2.90-2.87 (m, 1H), 2.60-2.55 (m, 1H), 2.40-2.35 (m, 1H), 1.99-1.96 (m, 1H). (ESI⁺) m/z: 479.2 (M+H)⁺, (C₂₅H₂₀F₂N₄O₄).

### Example 425

### Synthesis of 3-(5-(3-(2-chloro-4-methoxyphenyl)-1-(difluoromethyl)-1H-pyrazol-4-yl)-1-oxoisoindolin-2-yl)piperidine-2,6-dione

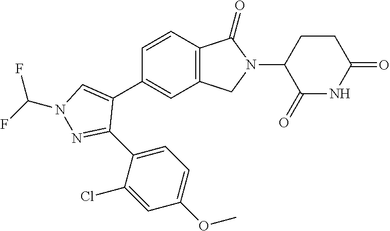 [SMILES: COc1ccc(-c2nn(C(F)F)cc2-c2ccc3c(c2)CN(C2CCC(=O)NC2=O)C3=O)c(Cl)c1]

A. 5-(2-Chloro-4-methoxyphenyl)-1H-pyrazole: To a solution of 5-bromo-1H-pyrazole (0.65 g, 4.42 mmol, 1.00 eq), 2-(2-chloro-4-methoxyphenyl)-4,4,5,5-tetramethyl-1,3,2-dioxaborolane (1.19 g, 4.42 mmol, 1.00 eq) and K₃PO₄ (1.88 g, 8.85 mmol, 2.00 eq) in dioxane (10.0 mL) and H₂O (1.00 mL) was added PdCl₂(dtbpf) (432 mg, 663 μmol, 0.15 eq) under N₂. The mixture was stirred at 90° C. for 12 h under N₂. The reaction mixture was poured into H₂O (50.0 mL) and extracted with Ethyl acetate (3×80.0 mL). Then the organic layer was dried over Na₂SO₄, filtered and the filtrate was concentrated to get residue. The residue was purified by column chromatography (SiO₂, Petroleum ether:Ethyl acetate=100:1 to 1:1, R_{f}=0.30 (Petroleum ether:Ethyl acetate=1:1) to give the title compound (550 mg, 2.94 mmol, 16.1% yield, 99.4% purity in LCMS at 220 nm) as black brown oil. (ESI⁺) m/z: 209.0 (M+H)⁺, (C₁₀H₉ClN₂O).

B. 4-Bromo-5-(2-chloro-4-methoxyphenyl)-1H-pyrazole: To a solution of 5-(2-chloro-4-methoxy-phenyl)-1H-pyrazole (300 mg, 1.44 mmol, 1.00 eq) in DMF (4.00 mL) was added NBS (255 mg, 1.44 mmol, 1.00 eq) at 0° C. under N₂. The mixture was stirred at 25° C. for 2 h under N₂. After the reaction was completed, the reaction mixture was filtered. The filtrate was poured into H₂O (20.0 mL) and extracted with ethyl acetate (3×20.0 mL). The organic layer was washed with brine (30.0 mL), dried over Na₂SO₄, filtered and the filtrate was concentrated in vacuum to get a residue. The residue was purified by column chromatography (SiO₂, Petroleum ether:Ethyl acetate=100:1 to 2:1, R_{f}=0.50 (Petroleum ether:Ethyl acetate=2:1)) to give the title compound (190 mg, 628 μmol, 43.7% yield, 95.1% purity in LCMS at 220 nm) as yellow oil. (ESI⁺) m/z: 286.9 (M+H)⁺, (C₁₀H₈BrClN₂O).

C. 4-Bromo-3-(2-chloro-4-methoxyphenyl)-1-(difluoromethyl)-1H-pyrazole: To a solution of 4-bromo-5-(2-chloro-4-methoxy-phenyl)-1H-pyrazole (190 mg, 628 μmol, 1.00 eq) and KF (73.0 mg, 1.26 mmol, 29.4 μL, 2.00 eq) in ACN (5.00 mL) was added 1-[[bromo(difluoro)methyl]-ethoxy-phosphoryl]oxyethane (184 mg, 691 μmol, 1.10 eq) at 25° C. under N₂. The mixture was stirred at 25° C. for 12 h under N₂. After the reaction was completed, the reaction mixture was filtered and the filtrate was the filtrate was concentrated under vacuum to give a residue. The residue was purified by preparative-TLC (Petroleum ether:Ethyl acetate=5:1, R_{f}=0.50) to give the title compound (80.0 mg, 236 μmol, 37.6% yield, 99.8% purity in HPLC at 220 nm) as white oil. ¹H NMR: (400 MHz, DMSO-d₆) δ 8.68 (s, 1H), 7.84 (t, J=58.8 Hz, 1H), 7.37 (d, J=8.8 Hz, 1H), 7.20 (d, J=2.4 Hz, 1H), 7.03 (dd, J=8.4, 2.4 Hz, 1H), 3.84 (s, 3H). (ESI⁺) m/z: 336.9 (M+H)⁺, (C₁₁H₈BrClF₂N₂O).

D. 3-(5-(3-(2-Chloro-4-methoxyphenyl)-1-(difluoromethyl)-1H-pyrazol-4-yl)-1-oxoisoindolin-2-yl)piperidine-2,6-dione: To a solution of 4-bromo-3-(2-chloro-4-methoxy-phenyl)-1-(difluoromethyl)pyrazole (70.0 mg, 207 μmol, 1.00 eq) in dioxane (2.00 mL) and H₂O (0.10 mL) was added 3-[1-oxo-5-(4,4,5,5-tetramethyl-1,3,2-dioxaborolan-2-yl)isoindolin-2-yl]piperidine-2,6-dione (84.4 mg, 228 μmol, 1.10 eq), Ru-Phos-Pd-G₃ (17.3 mg, 20.7 μmol, 0.10 eq) and K₃PO₄ (88.0 mg, 414 μmol, 2.00 eq) under N₂. The mixture was stirred at 100° C. for 2 h under N₂. After the reaction was completed, the reaction mixture was filtered and the filtrate was concentrated under vacuum to give a residue. The residue was purified by preparative-TLC (Dichloromethane:Methanol=10:1, R_{f}=0.50) and by preparative-HPLC (using a Welch Xtimate C18 (150 mm×25 mm×5 μm) and gradient of 30-60% acetonitrile in water containing 0.5% TFA over 10 min at a flow rate of 25.0 mL/min) to give the title compound (16.5 mg, 32.9 μmol, 99.7% purity in HPLC at 220 nm) as a white solid. ¹H NMR: (400 MHz, DMSO-d₆) δ 10.9 (s, 1H), 8.77 (s, 1H), 7.91 (t, J=59.2 Hz, 1H), 7.62 (d, J=8.0 Hz, 1H), 7.44 (t, J=3.2 Hz, 2H), 7.26 (dd, J=8.0, 1.2 Hz, 1H), 7.13 (d, J=2.4 Hz, 1H), 7.04 (dd, J=8.4, 2.8 Hz, 1H), 5.09 (dd, J=12.8, 4.8 Hz, 1H), 4.32 (dd, J=57.6, 17.6 Hz, 2H), 3.83 (s, 3H), 2.92-2.87 (m, 1H), 2.61-2.59 (m, 1H), 2.41-2.36 (m, 1H), 2.00-1.96 (m, 1H). (ESI⁺) m/z: 501.1 (M+H)⁺, (C₂₄H₁₉ClF₂N₄O₄).

### Example 426

### Synthesis of 3-(5-(1-isopropyl-5-phenyl-1H-pyrazol-4-yl)-1-oxoisoindolin-2-yl)piperidine-2,6-dione

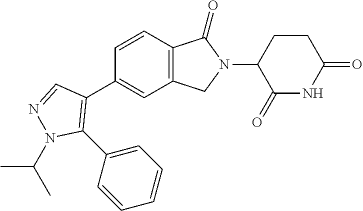 [SMILES: CC(C)n1ncc(-c2ccc3c(c2)CN(C2CCC(=O)NC2=O)C3=O)c1-c1ccccc1]

A. 4-bromo-1-isopropyl-5-phenyl-1H-pyrazole: To a solution of 4-bromo-1-isopropyl-pyrazole (200 mg, 1.06 mmol, 1.00 eq) in DMF (4.00 mL) was added bromobenzene (996 mg, 6.35 mmol, 668 μL, 6.00 eq), P(oxole)₃ (49.1 mg, 211 μmol, 0.20 eq), K₂CO₃ (292 mg, 2.12 mmol, 2.00 eq) and Pd(OAc)₂ (23.7 mg, 105 μmol, 0.10 eq) under N₂. The mixture was stirred at 100° C. for 12 h under N₂. After the reaction was completed, the reaction mixture was filtered. The filtrate was poured into H₂O (10.0 mL) and extracted with ethyl acetate (3×20.0 mL). The organic layer was washed with brine (30.0 mL), dried over Na₂SO₄, filtered and the filtrate was concentrated under vacuum to get a residue. The residue was purified by preparative-TLC (Petroleum ether: ethyl acetate=3:1, R_{f}=0.60) to give the title compound (70.0 mg, 193 μmol, 18.2% yield, 73.3% purity in LCMS at 220 nm) as yellow oil. ¹H NMR: (400 MHz, DMSO-d₆) δ 7.69 (s, 1H), 7.58-7.50 (m, 3H), 7.43-7.40 (m, 2H), 4.40-4.32 (m, 1H), 1.33 (d, J=6.8 Hz, 6H). (ESI⁺) m/z: 265.0 (M+H)⁺, (C₁₂H₁₃BrN₂).

B. 3-(5-(1-Isopropyl-5-phenyl-1H-pyrazol-4-yl)-1-oxoisoindolin-2-yl)piperidine-2,6-dione: To a solution of 4-bromo-1-isopropyl-5-phenyl-pyrazole (70.0 mg, 193 μmol, 1.00 eq) in dioxane (2.00 mL) and H₂O (0.10 mL) was added 3-[1-oxo-5-(4,4,5,5-tetramethyl-1,3,2-dioxaborolan-2-yl)isoindolin-2-yl]piperidine-2,6-dione (179 mg, 483 μmol, 2.50 eq), Ru-Phos-Pd-G₃ (16.1 mg, 19.3 μmol, 0.10 eq) and K₃PO₄ (82.1 mg, 387 μmol, 2.00 eq) under N₂. The mixture was stirred at 100° C. for 2 h under N₂. After the reaction was completed, the reaction mixture was filtered and the filtrate was concentrated in vacuum to get a residue. The residue was purified by preparative-TLC (SiO₂, DCM:Methanol=10:1, R_{f}=0.50) and then by preparative-HPLC (using a Welch Xtimate C18 (150 mm×25 mm×5 μm) and gradient of 30-60% acetonitrile in water containing 0.5% TFA over 10 min at a flow rate of 25.0 mL/min) to give the title compound (31.1 mg, 72.4 μmol, 37.4% yield, 99.7% purity in HPLC at 220 nm) as a white solid. ¹H NMR: (400 MHz, DMSO-d₆) δ 10.9 (s, 1H), 7.96 (s, 1H), 7.55-7.50 (m, 4H), 7.39-7.35 (m, 3H), 7.20 (d, J=8.0 Hz, 1H), 5.06 (dd, J=13.2, 5.2 Hz, 1H), 4.35-4.15 (m, 3H), 2.91-2.84 (m, 1H), 2.59 (s, 1H), 2.38 (d, J=3.6 Hz, 1H), 1.99-1.94 (m, 1H), 1.37 (d, J=6.4 Hz, 6H). (ESI⁺) m/z: 429.1 (M+H)⁺, (C₂₅H₂₄N₄O₃).

### Example 427

### Synthesis of 3-(1-oxo-5-(3-phenyl-1-(2,2,2-trifluoroethyl)-1H-pyrazol-4-yl)isoindolin-2-yl)piperidine-2,6-dione

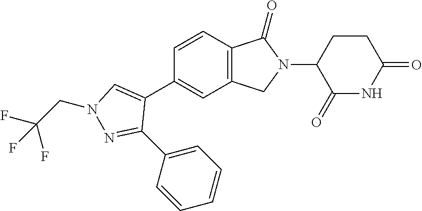 [SMILES: O=C1CCC(N2Cc3cc(-c4cn(CC(F)(F)F)nc4-c4ccccc4)ccc3C2=O)C(=O)N1]

A. 4-Bromo-3-phenyl-1-(2,2,2-trifluoroethyl)-1H-pyrazole: To a solution of 4-bromo-5-phenyl-1H-pyrazole (200 mg, 896 μmol, 1.00 eq) and Cs₂CO₃ (876 mg, 2.69 mmol, 3.00 eq) in DMF (4.00 mL) was added dropwise 2,2,2-trifluoroethyl trifluoromethanesulfonate (228 mg, 986 μmol, 1.10 eq). The mixture was stirred at 25° C. for 2 h. Then the reaction mixture was quenched with 5.00 mL of water and filtered. The filtrate was concentrated in vacuum to get a residue. The residue was purified by preparative-TLC (SiO₂, Petroleum ether:EtOAc=10:1, R_{f}=0.60) to give the title compound (230 mg, 753 μmol, 84.0% yield) as a white solid. ¹H NMR: (400 MHz, DMSO-d₆) δ 8.22 (s, 1H), 7.82-7.78 (m, 2H), 7.51-7.46 (m, 2H), 7.45-7.42 (m, 1H), 5.20 (dd, J=18.4, 9.2 Hz, 2H), (ESI⁺) m/z: 304.9 (M+H)⁺, (C₁₁H₈BrF₃N₂).

B. tert-butyl 5-amino-5-oxo-4-(1-oxo-5-(3-phenyl-1-(2,2,2-trifluoroethyl)-1H-pyrazol-4-yl)isoindolin-2-yl)pentanoate: To a solution of 4-bromo-3-phenyl-1-(2,2,2-trifluoroethyl)pyrazole (180 mg, 589 μmol, 1.00 eq) and tert-butyl 5-amino-5-oxo-4-[1-oxo-5-(4,4,5,5-tetramethyl-1,3,2-dioxaborolan-2-yl)isoindolin-2-yl]pentanoate (393 mg, 884 μmol, 1.50 eq) in dioxane (2.00 mL) and H₂O (0.10 mL) was added K₃PO₄ (375 mg, 1.77 mmol, 3.00 eq) and Ru-Phos-Pd-G₃ (98.6 mg, 118 μmol, 0.20 eq). The mixture was stirred at 100° C. for 2 h under N₂. Then the reaction mixture was filtered and the filtrate was concentrated in vacuum to get a residue. The residue was purified by preparative-TLC (SiO₂, DCM:Methanol=10:1, R_{f}=0.60) to give the title compound (300 mg, 531 μmol, 90.1% yield, 96.2% purity in LCMS at 220 nm) as a white solid. (ESI⁺) m/z: 543.3 (M+H)⁺, (C₂₈H₂₉F₃N₄O₄).

C. 3-(1-Oxo-5-(3-phenyl-1-(2,2,2-trifluoroethyl)-1H-pyrazol-4-yl)isoindolin-2-yl)piperidine-2,6-dione: To a solution of tert-butyl 5-amino-5-oxo-4-[1-oxo-5-[3-phenyl-1-(2,2,2-trifluoroethyl)pyrazol-4-yl]isoindolin-2-yl]pentanoate (300 mg, 531 μmol, 1.00 eq) in ACN (3.00 mL) was added TsOH (916 mg, 5.32 mmol, 10.0 eq). The mixture was stirred at 80° C. for 2 h. Then the reaction mixture was poured into 5.00 mL of water and extracted with EtOAc (3×5.00 mL). The combined organic layer was washed with brine (5.00 mL), dried over Na₂SO₄ and filtered. The filtrate was concentrated in vacuum to get a residue. The residue was purified by preparative-TLC (SiO₂, DCM:Methanol=10:1, R_{f}=0.60) and then by preparative-HPLC (using a Phenomenex Luna C18 (150 mm×30 mm 5 μm) and gradient of 30-60% acetonitrile in water containing 0.5% HCl over 10 min at a flow rate of 25.0 mL/min) to give the title compound (96.8 mg, 200 μmol, 37.7% yield, 97.2% purity in HPLC at 220 nm) as a white solid. ¹H NMR: (400 MHz, DMSO-d₆) δ 11.0 (br, 1H), 8.2 (s, 1H), 7.66 (d, J=8.0 Hz, 1H), 7.50 (s, 1H), 7.42-7.32 (m, 6H), 5.24 (dd, J=18.0, 9.2 Hz, 2H), 5.11 (dd, J=13.2, 5.2 Hz, 1H), 4.35 (dd, J=52.8, 17.2 Hz, 2H), 2.97-2.85 (m, 1H), 2.62-2.56 (m, 1H), 2.45-2.31 (m, 1H), 2.05-1.96 (m, 1H). (ESI⁺) m/z: 469.2 (M+H)⁺, (C₂₄H₁₉F₃N₄O₃).

### Example 428

### Synthesis of 3-(5-(5-(2,2-difluorobenzo[d][1,3]dioxol-5-yl)-1-methyl-1H-pyrazol-4-yl)-1-oxoisoindolin-2-yl)piperidine-2,6-dione

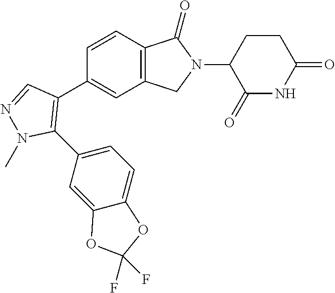 [SMILES: Cn1ncc(-c2ccc3c(c2)CN(C2CCC(=O)NC2=O)C3=O)c1-c1ccc2c(c1)OC(F)(F)O2]

A. tert-Butyl 5-amino-4-(5-(5-(2,2-difluorobenzo[d][1,3]dioxol-5-yl)-1-methyl-1H-pyrazol-4-yl)-1-oxoisoindolin-2-yl)-5-oxopentanoate: To a solution of (2,2-difluorobenzo[d][1,3]dioxol-5-yl)boronic acid (159 mg, 785 μmol, 1.50 eq), tert-butyl5-amino-4-(5-(5-bromo-1-methyl-1H-pyrazol-4-yl)-1-oxoisoindolin-2-yl)-5-oxopentanoate (250 mg, 523 μmol, 1.00 eq) and K₃PO₄ (333 mg, 1.57 mmol, 3.00 eq) in dioxane (10.0 mL) and H₂O (2.50 mL) was added Pd(dtbpf)Cl₂ (34.1 mg, 52.3 μmol, 0.10 eq) under N₂. The mixture was stirred at 65° C. for 16 h under N₂. The mixture was filtered to collect liquid and concentrated under reduced pressure to get residue. The residue was purified by column chromatography (SiO₂, Dichloromethane:Methanol=10:1, R_{f}=0.25) to get the title compound (450 mg, 765 μmol, 73.0% yield, 94.3% purity at 220 nm in LCMS) as yellow solid. ¹H NMR: (400 MHz, DMSO-d₆) δ 7.86 (s, 1H), 7.65-7.52 (m, 5H), 7.51-7.43 (m, 1H), 7.22-7.17 (m, 2H), 4.70-4.67 (m, 1H), 4.54-4.34 (m, 2H), 3.48 (s, 3H), 2.21-1.90 (m, 4H), 1.33 (m, 9H). (ESI⁺) m/z: 555.2 (M+H)⁺, (C₂₈H₂₈F₂N₄O₆).

B. 3-(5-(5-(2,2-Difluorobenzo[d][1,3]dioxol-5-yl)-1-methyl-1H-pyrazol-4-yl)-1-oxoisoindolin-2-yl)piperidine-2,6-dione: To a solution of tert-butyl 5-amino-4-(5-(5-(2,2-difluorobenzo[d][1,3]dioxol-5-yl)-1-methyl-1H-pyrazol-4-yl)-1-oxoisoindolin-2-yl)-5-oxopentanoate (200 mg, 360 μmol, 1.00 eq) in ACN (10.0 mL) was added TsOH (248 mg, 1.44 mmol, 4.00 eq). The mixture was stirred at 70° C. for 4 h. The mixture was filtered to collect liquid and concentrated under reduced pressure to get residue. The crude product was purified by preparative-HPLC (using a Phenomenex luna C18 (150 mm×25 mm 10 μm) and gradient of 12-42% acetonitrile in water containing 0.05% FA over 15 min at a flow rate of 25 mL/min) to give the title compound (82.2 mg, 163 μmol, 47.7% yield, 95.7% purity in HPLC at 220 nm) as white solid. ¹H NMR: (400 MHz, DMSO-d₆) δ 10.9 (s, 1H), 7.87 (s, 1H), 7.62-7.59 (m, 1H), 7.57-7.52 (m, 3H), 7.50-7.47 (m, 1H), 7.22-7.20 (m, 1H), 5.11-5.03 (m, 1H), 4.39-4.20 (m, 2H), 3.49 (s, 3H), 3.03-3.00 (m, 2H), 2.94-2.85 (m, 1H), 2.60-2.55 (m, 1H), 2.37-2.33 (m, 1H), 1.98-1.96 (m, 1H). (ESI⁺) m/z: 481.0 (M+H)⁺, (C₂₄H₁₈F₂N₄O₅).

### Example 429

### Synthesis 3-(5-(1-ethyl-5-phenyl-1H-pyrazol-4-yl)-1-oxoisoindolin-2-yl)piperidine-2,6-dione

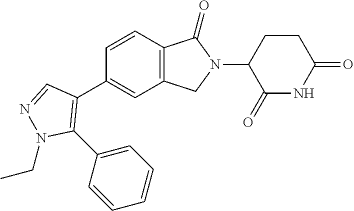 [SMILES: CCn1ncc(-c2ccc3c(c2)CN(C2CCC(=O)NC2=O)C3=O)c1-c1ccccc1]

A. 4-Bromo-1-ethyl-5-phenyl-1H-pyrazole: To a solution of 4-bromo-1-ethyl-1H-pyrazole (1.00 g, 5.71 mmol, 1.00 eq), bromobenzene (4.49 g, 28.5 mmol, 3.01 mL, 5.00 eq), K₂CO₃ (1.58 g, 11.43 mmol, 2.00 eq) in DMF (15.0 mL) was added P(oxole)₃ (265 mg, 1.14 mmol, 0.20 eq) and Pd(OAc)₂ (256 mg, 1.14 mmol, 0.20 eq) under N₂. The mixture was stirred at 100° C. for 48 h under N₂. The mixture was poured into H₂O (100 mL) and extracted with Ethyl acetate (3×40.0 mL). The combined organic layer was washed with saturated NaCl aqueous (4×50.0 mL), dried over Na₂SO₄ and concentrated under reduced pressure to get residue. The residue was purified by column chromatography (SiO₂, Petroleum ether: Ethyl acetate=10:1, R_{f}=0.45) and preparative-TLC (Dichloromethane: Petroleum ether=2:1, R_{f}=0.60) to get title compound (700 mg, 2.76 mmol, 23.1% yield, 99.1% purity at 220 nm in LCMS) as light yellow solid. ¹H NMR: (400 MHz, DMSO-d₆) δ 7.67 (s, 1H), 7.58-7.43 (m, 5H), 4.06-4.01 (m, 2H), 1.24-1.21 (m, 3H). (ESI⁺) m/z: 253.0 (M+H)⁺, (C₁₁H₁₁BrN₂).

B. 3-(5-(1-Ethyl-5-phenyl-1H-pyrazol-4-yl)-1-oxoisoindolin-2-yl)piperidine-2,6-dione: To a solution of 4-bromo-1-ethyl-5-phenyl-1H-pyrazole (200 mg, 796 μmol, 1.00 eq), 3-(1-oxo-5-(4,4,5,5-tetramethyl-1,3,2-dioxaborolan-2-yl)isoindolin-2-yl)piperidine-2,6-dione (589 mg, 1.59 mmol, 2.00 eq) and K₃PO₄ (338 mg, 1.59 mmol, 2.00 eq) in dioxane (5.00 mL) and H₂O (0.25 mL) was added Ru-Phos-Pd-G₃ (133 mg, 159 μmol, 0.20 eq) under N₂. The mixture was stirred at 100° C. for 2 h under N₂. The mixture was filtered to collect liquid and concentrated under reduced pressure to get residue. The crude product was purified by preparative-HPLC (using a Phenomenex luna C18 (150 mm×25 mm 10 μm) and gradient of 22-52% acetonitrile in water containing 0.5% TFA over 15 min at a flow rate of 25 mL/min) to give the title compound (295 mg, 708 μmol, 89.0% yield, 99.6% purity in HPLC at 220 nm) as white solid. ¹H NMR: (400 MHz, DMSO-d₆) δ 10.9 (s, 1H), 7.94 (s, 1H), 7.54-7.39 (m, 4H), 7.38-7.36 (m, 3H), 7.22-7.20 (m, 1H), 5.09-5.02 (m, 1H), 4.35-4.16 (m, 2H), 4.02-3.95 (m, 2H), 2.90-2.86 (m, 1H), 2.59-2.54 (m, 1H), 2.38-2.34 (m, 1H), 1.99-1.96 (m, 1H), 1.28-1.24 (m, 3H). (ESI⁺) m/z: 415.1 (M+H)⁺, (C₂₄H₂₂N₄O₃).

### Example 430

### Synthesis of 3-(5-(1-(2,2-difluoroethyl)-5-phenyl-1H-pyrazol-4-yl)-1-oxoisoindolin-2-yl)piperidine-2,6-dione

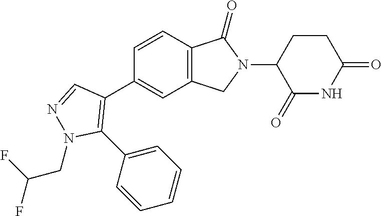 [SMILES: O=C1CCC(N2Cc3cc(-c4cnn(CC(F)F)c4-c4ccccc4)ccc3C2=O)C(=O)N1]

A. 4-Bromo-1-(2,2-difluoroethyl)-1H-pyrazole: To a solution of 4-bromo-1H-pyrazole (5.00 g, 34.0 mmol, 1.00 eq) in DMF (100 mL) was added 1,1-difluoro-2-iodo-ethane (7.84 g, 40.8 mmol, 1.20 eq) and K₂CO₃ (9.40 g, 68.0 mmol, 2.00 eq) under N₂. The mixture was stirred at 80° C. for 16 h under N₂. After the reaction was completed, the reaction mixture was cooled to 25° C. and filtered. The filtrate was poured into H₂O (100 mL) and extracted with ethyl acetate (3×100 mL). The organic layer was washed with brine (100 mL), dried over Na₂SO₄, filtered and the filtrate was concentrated in vacuum to give the title compound (6.00 g, 28.0 mmol, 82.5% yield, 98.8% purity in LCMS at 220 nm) as yellow oil. ¹H NMR: (400 MHz, DMSO-d₆) δ 8.02 (s, 1H), 7.64 (s, 1H), 6.49-6.20 (m, 1H), 4.67-4.58 (m, 2H). (ESI⁺) m/z: 210.9 (M+H)⁺, (C₅H₅BrF₂N₂).

B. 4-Bromo-1-(2,2-difluoroethyl)-5-phenyl-1H-pyrazole: To a solution of 4-bromo-1-(2,2-difluoroethyl)pyrazole (1.00 g, 4.68 mmol, 1.00 eq) and bromobenzene (735 mg, 4.68 mmol, 493 μL, 1.00 eq) in NMP (15.0 mL) was added Pd(OAc)₂ (10.5 mg, 46.8 μmol, 0.01 eq), DavePhos (36.8 mg, 93.6 μmol, 0.02 eq), Bu₄NOAc (2.82 g, 9.36 mmol, 2.85 mL, 2.00 eq) and isobutric acid (123 mg, 1.40 mmol, 130 μL, 0.30 eq) under N₂. The mixture was stirred at 100° C. for 16 h under N₂. After the reaction was completed, the reaction mixture was filtered and the filtrate was concentrated in vacuum to get a residue. The residue was purified by reversed-phase HPLC (0.1% NH₃H₂O) to give the title compound (220 mg, 736 μmol, 15.7% yield, 96.1% purity in LCMS at 220 nm) as yellow oil. ¹H NMR: (400 MHz, DMSO-d₆) δ 7.81 (s, 1H), 7.58-7.54 (m, 3H), 7.45-7.43 (m, 2H), 6.45-6.15 (m, 1H), 4.53-4.44 (m, 2H). (ESI⁺) m/z: 286.9 (M+H)⁺, (C₁₁H₉BrF₂N₂).

C. 3-(5-(1-Isopropyl-5-phenyl-1H-1,2,4-triazol-3-yl)-1-oxoisoindolin-2-yl)piperidine-2,6-dione: To a solution of 4-bromo-1-(2,2-difluoroethyl)-5-phenyl-pyrazole (200 mg, 669 μmol, 1.00 eq) and 3-[1-oxo-5-(4,4,5,5-tetramethyl-1,3,2-dioxaborolan-2-yl)isoindolin-2-yl]piperidine-2,6-dione (619 mg, 1.67 mmol, 2.50 eq) in dioxane (1.00 mL) and H₂O (0.05 mL) was added Ru-Phos-Pd-G₃ (55.9 mg, 66.9 μmol, 0.10 eq) and K₃PO₄ (284 mg, 1.34 mmol, 2.00 eq) under N₂. The mixture was stirred at 100° C. for 2 h under N₂.

After the reaction was completed, the reaction mixture was filtered and the filtrate was concentrated in vacuum to give a residue. The residue was purified by preparative-TLC (Dichloromethane:Methanol=10:1, R_{f}=0.50) and by preparative-HPLC (using a Phenomenex Luna C18 (250 mm×50 mm×10 μm) and gradient of 25-55% acetonitrile in water containing 0.5% TFA over 10 min at a flow rate of 25.0 mL/min) to give the title compound (193 mg, 426 μmol, 64.2% yield, 99.2% purity in HPLC at 220 nm) as a white solid. ¹H NMR: (400 MHz, DMSO-d₆) δ 10.96 (s, 1H), 8.06 (s, 1H), 7.56-7.53 (m, 4H), 7.39-7.36 (m, 3H), 7.23 (d, J=8.0 Hz, 1H), 6.50-6.20 (m, 1H), 5.07 (dd, J=13.2, 4.8 Hz, 1H), 4.46-4.37 (m, 2H), 4.37 (dd, J=23.2, 17.2 Hz, 2H), 2.91-2.88 (m, 1H), 2.59 (s, 1H), 2.39-2.32 (m, 1H), 1.98-1.94 (m, 1H). (ESI⁺) m/z: 451.1 (M+H)⁺, (C₂₄H₂₀F₂N₄O₃).

### Example 431

### Synthesis of 3-(5-(5-(3-fluoro-4-methoxyphenyl)-1-(methyl-d₃)-1H-pyrazol-4-yl)-1-oxoisoindolin-2-yl)piperidine-2,6-dione

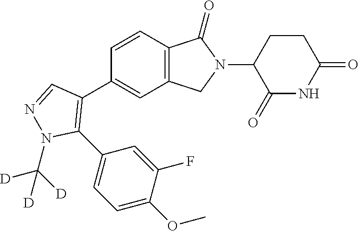 [SMILES: [2H]C([2H])([2H])n1ncc(-c2ccc3c(c2)CN(C2CCC(=O)NC2=O)C3=O)c1-c1ccc(OC)c(F)c1]

A. 4-Bromo-5-(3-fluoro-4-methoxyphenyl)-1-(methyl-d₃)-1H-pyrazole: To a solution of 4-bromo-1-(methyl-d₃)-1H-pyrazole (500 mg, 3.05 mmol, 1.00 eq), 4-bromo-2-fluoro-1-methoxybenzene (2.50 g, 12.2 mmol, 4.00 eq) and K₂CO₃ (843 mg, 6.10 mmol, 2.00 eq) in DMF (7.50 mL) was added P(oxole)₃ (142 mg, 610 μmol, 0.20 eq), Pd(OAc)₂ (205 mg, 914 μmol, 0.30 eq) under N₂. The mixture was stirred at 110° C. for 48 h under N₂. The mixture was poured into H₂O (70.0 mL) and extracted with Ethyl acetate (3×30.0 mL). The combined organic layer was washed with saturated NaCl aqueous (4×50.0 mL), dried over Na₂SO₄ and concentrated under reduced pressure to get residue. The residue was purified by preparative-TLC (Petroleum ether/Ethyl acetate=2/1, R_{f}=0.25) to get the title compound (300 mg, 1.04 mmol, 34.1% yield, 85.6% purity in LCMS at 220 nm) as black brown solid. ¹H NMR: (400 MHz, DMSO-d₆) δ 7.63 (s, 1H), 7.43-7.40 (m, 1H), 7.34-7.29 (m, 2H), 3.92 (s, 3H). (ESI⁺) m/z: 290.0 (M+H)⁺, (C₁₁H₇D₃BrFN₂O).

B. 3-(5-(5-(3-fluoro-4-methoxyphenyl)-1-(methyl-d₃)-1H-pyrazol-4-yl)-1-oxoisoindolin-2-yl)piperidine-2,6-dione: To a solution of 4-bromo-5-(3-fluoro-4-methoxyphenyl)-1-(methyl-d₃)-1H-pyrazole (120 mg, 416 μmol, 1.00 eq), 3-(1-oxo-5-(4,4,5,5-tetramethyl-1,3,2-dioxaborolan-2-yl)isoindolin-2-yl)piperidine-2,6-dione (308 mg, 832 μmol, 2.00 eq) and K₃PO₄ (177 mg, 833 μmol, 2.00 eq) in dioxane (5.00 mL) and H₂O (0.25 mL) was added Ru-Phos-Pd-G₃ (69.7 mg, 83.3 μmol, 0.20 eq) under N₂. The mixture was stirred at 100° C. for 2 h under N₂. The mixture was filtered to collect liquid and concentrated under reduced pressure to get residue. The crude product was purified by preparative-HPLC (using a Phenomenex luna C18 (150 mm×25 mm×10 μm) and gradient of 25-55% acetonitrile in water containing 0.05% FA over 15 min at a flow rate of 25 mL/min) to give the title compound (132 mg, 288 μmol, 69.2% yield, 98.6% purity in HPLC at 220 nm) as white solid. ¹H NMR: (400 MHz, DMSO-d₆) δ 10.9 (s, 1H), 7.88 (s, 1H), 7.59-7.40 (m, 1H), 7.37-7.25 (m, 4H), 7.23-7.12 (m, 1H), 5.10-5.05 (m, 1H), 4.37-4.20 (m, 2H), 3.89 (s, 3H), 2.91-2.60 (m, 1H), 2.59-2.55 (m, 1H), 2.39-2.35 (m, 1H), 1.99-1.96 (m, 1H). Deuterated ratio: 98.6%. (ESI⁺) m/z: 452.3 (M+H)⁺, (C₂₄H₁₈D₃FN₄O₄).

### Example 432

### Synthesis of 3-(5-(5-(2,3-dihydrobenzofuran-5-yl)-1-(methyl-d₃)-1H-pyrazol-4-yl)-1-oxoisoindolin-2-yl)piperidine-2,6-dione

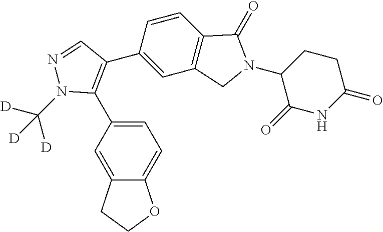 [SMILES: [2H]C([2H])([2H])n1ncc(-c2ccc3c(c2)CN(C2CCC(=O)NC2=O)C3=O)c1-c1ccc2c(c1)CCO2]

A. 4-Bromo-5-(2, 3-dihydrobenzofuran-5-yl)-1-(methyl-d₃)-1H-pyrazole: To a solution of 4-bromo-1-(methyl-d₃)-1H-pyrazole (500 mg, 3.05 mmol, 1.00 eq) and 5-bromo-2,3-dihydrobenzofuran (2.43 g, 12.2 mmol, 4.00 eq) in DMF (7.50 mL) was added K₂CO₃ (842 mg, 6.10 mmol, 2.00 eq), P(oxole)₃ (141 mg, 609 μmol, 0.20 eq) and Pd(OAc)₂ (205 mg, 914 μmol, 0.30 eq) under N₂. The mixture was stirred at 100° C. for 40 h under N₂. The reaction mixture was poured into H₂O (30.0 mL) and extracted with Ethyl acetate (3×50.0 mL). The combined organic layer was washed with brine (3×50.0 mL), and dried over by Na₂SO₄, filtered and concentrate filtrate under reduced pressure to get residue. The residue was purified by column chromatography (SiO₂, Petroleum ether:Ethyl acetate=50:1, TLC: Petroleum ether:Ethyl acetate=1:1, R_{f}=0.50) to give the title compound (280 mg, 823 μmol, 27.1% yield, 83.0% purity in LCMS at 220 nm) as black brown oil. ¹H NMR: (400 MHz, DMSO-d₆) δ 7.60-7.56 (m, 1H), 7.33-7.13 (m, 2H), 6.92-6.90 (m, 1H), 4.62-4.53 (m, 2H), 3.33-3.23 (m, 2H). (ESI⁺) m/z: 283.7 (M+H)⁺, (C₁₂H₈D₃BrN₂O).

B. 3-(5-(5-(2,3-Dihydrobenzofuran-5-yl)-1-(methyl-d₃)-1H-pyrazol-4-yl)-1-oxoisoindolin-2-yl)piperidine-2,6-dione: To a solution of 4-bromo-5-(2,3-dihydrobenzofuran-5-yl)-1-(methyl-d₃)-1H-pyrazole (150 mg, 531 μmol, 1.00 eq), 3-(1-oxo-5-(4,4,5,5-tetramethyl-1,3,2-dioxaborolan-2-yl)isoindolin-2-yl)piperidine-2,6-dione (590 mg, 1.59 mmol, 3.00 eq) and K₃PO₄ (338 mg, 1.59 mmol, 3.00 eq) in dioxane (5.00 mL) and H₂O (0.25 mL) was added Ru-Phos-Pd-G₃ (44.4 mg, 53.1 μmol, 0.10 eq) under N₂. The mixture was stirred at 100° C. for 4 h under N₂. The reaction mixture was concentrated under reduced pressure to get a residue at 45° C. The residue was purified by preparative-HPLC (using a Phenomenex luna C18 (150×25 mm×5 μm) and gradient of 21.0%-51.0% acetonitrile in water containing 0.05% FA over 10 min at a flow rate of 25 mL/min) to give the title compound (46.4 mg, 104 μmol, 19.5% yield, 99.8% purity in HPLC at 220 nm) as white solid. ¹H NMR: (400 MHz, DMSO-d₆) δ 10.9 (s, 1H), 7.87 (s, 1H), 7.58-7.54 (m, 1H), 7.43 (s, 1H), 7.25-7.23 (m, 2H), 7.08-7.05 (m, 1H), 6.88-6.86 (m, 1H), 5.09-5.05 (m, 1H), 4.62-4.58 (m, 2H), 4.38-4.52 (m, 2H), 3.24-3.20 (m, 2H), 2.90-2.86 (m, 1H), 2.60-2.55 (m, 1H), 2.39-2.35 (m, 1H), 1.98-1.95 (m, 1H). Deuterated ratio: 96.5%. (ESI⁺) m/z: 446.2 (M+H)⁺, (C₂₅H₁₉D₃N₄O₄).

### Example 433

### Synthesis of 3-(5-(5-(4-methoxyphenyl)-1-(methyl-d₃)-1H-pyrazol-4-yl)-1-oxoisoindolin-2-yl) piperidine-2,6-dione

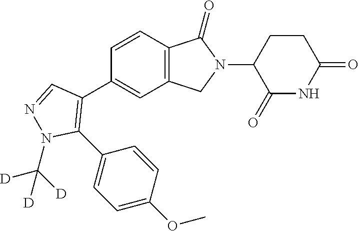 [SMILES: [2H]C([2H])([2H])n1ncc(-c2ccc3c(c2)CN(C2CCC(=O)NC2=O)C3=O)c1-c1ccc(OC)cc1]

A. 4-Bromo-5-(4-methoxyphenyl)-1-(methyl-d₃)-1H-pyrazole: To a solution of 4-bromo-1-(methyl-d₃)-1H-pyrazole (500 mg, 3.05 mmol, 1.00 eq), 4-bromo-2-fluoro-1-methoxybenzene (2.28 g, 12.2 mmol, 1.53 mL, 4.00 eq) and K₂CO₃ (843 mg, 6.10 mmol, 2.00 eq) in DMF (7.50 mL) was added P(oxole)₃ (142 mg, 610 μmol, 0.20 eq), Pd(OAc)₂ (205 mg, 914 μmol, 0.30 eq) under N₂. The mixture was stirred at 110° C. for 48 h under N₂. The mixture was poured into H₂O (70.0 mL) and extracted with Ethyl acetate (3×30.0 mL). The combined organic layer was washed with saturated NaCl aqueous (4×50.0 mL), dried over Na₂SO₄ and concentrated under reduced pressure to get residue. The residue was purified by preparative-TLC (Petroleum ether/Ethyl acetate=2/1, R_{f}=0.25) to get the title compound (300 mg, 1.11 mmol, 36.4% yield, 96.1% purity in LCMS at 220 nm) as black brown solid. ¹H NMR: (400 MHz, DMSO-d₆) δ 7.61 (s, 1H), 7.42-7.40 (m, 2H), 7.11-7.08 (m, 2H), 3.82 (s, 3H). Deuterated ratio: 97.2%. (ESI⁺) m/z: 270.0 (M+H)⁺, (C₁₁H₈D₃BrN₂O).

B. 3-(5-(5-(4-Methoxyphenyl)-1-(methyl-d₃)-1H-pyrazol-4-yl)-1-oxoisoindolin-2-yl)piperidine-2,6-dione: To a solution of 4-bromo-5-(4-methoxyphenyl)-1-(methyl-d₃)-1H-pyrazole (120 mg, 444 μmol, 1.00 eq), 3-(1-oxo-5-(4,4,5,5-tetramethyl-1,3,2-dioxaborolan-2-yl)isoindolin-2-yl)piperidine-2,6-dione (328 mg, 888 μmol, 2.00 eq) and K₃PO₄ (188 mg, 888 μmol, 2.00 eq) in dioxane (5.00 mL) and H₂O (0.25 mL) was added Ru-Phos-Pd-G₃ (74.3 mg, 88.8 μmol, 0.20 eq) under N₂. The mixture was stirred at 100° C. for 2 h under N₂. The mixture was filtered to collect liquid and concentrated under reduced pressure to get residue. The crude product was purified by preparative-HPLC (using a Phenomenex luna C18 (150 mm×25 mm×10 μm) and gradient of 54-84% acetonitrile in water containing 0.05% FA over 15 min at a flow rate of 25 mL/min) to give the title compound (79.0 mg, 181 μmol, 40.7% yield, 99.3% purity in HPLC at 220 nm) as white solid. ¹H NMR: (400 MHz, DMSO-d₆) δ 10.9 (s, 1H), 7.87 (s, 1H), 7.56-7.54 (m, 1H), 7.39-7.32 (m, 1H), 7.30-7.26 (m, 2H), 7.24-7.22 (m, 1H), 7.07-7.05 (m, 2H), 5.09-5.05 (m, 1H), 4.37-4.18 (m, 2H), 3.81 (s, 3H), 2.91-2.86 (m, 1H), 2.60-2.55 (m, 1H), 2.39-2.34 (m, 1H), 1.99-1.96 (m, 1H). Deuterated ratio (ESI⁺) m/z: 434.1 (M+H)⁺, (C₂₄H₁₉D₃N₄O₄).

### Example 434

### Synthesis of 3-(5-(5-(2-chloro-4-methoxyphenyl)-1-(methyl-d₃)-1H-pyrazol-4-yl)-1-oxoisoindolin-2-yl)piperidine-2,6-dione

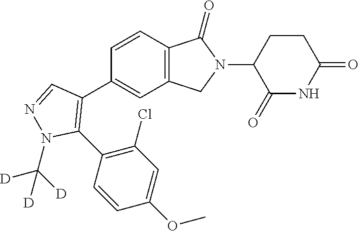 [SMILES: [2H]C([2H])([2H])n1ncc(-c2ccc3c(c2)CN(C2CCC(=O)NC2=O)C3=O)c1-c1ccc(OC)cc1Cl]

A. 4-Bromo-5-(2-chloro-4-methoxyphenyl)-1-(methyl-d₃)-1H-pyrazole: To a solution of 4-bromo-1-(methyl-d₃)-1H-pyrazole (500 mg, 3.05 mmol, 1.00 eq) and 1-bromo-2-chloro-4-methoxybenzene (2.70 g, 12.2 mmol, 4.00 eq) in DMF (7.50 mL) was added K₂CO₃ (842 mg, 6.10 mmol, 2.00 eq), P(oxole)₃ (141 mg, 609 μmol, 0.20 eq) and Pd(OAc)₂ (136 mg, 609 μmol, 0.20 eq) under N₂. The mixture was stirred at 100° C. for 72 h under N₂. The reaction mixture was poured into H₂O (10.0 mL) and extracted with Ethyl acetate (3×20.0 mL). The combined organic layer was washed with brine (3×20.0 mL), and dried over by Na₂SO₄, filtered and concentrate filtrate under reduced pressure to get residue. The residue was purified by preparative-TLC (Petroleum ether:Dichloromethane=1:1,R_{f}=0.40) to give the title compound (120 mg, 367 μmol, 12.0% yield, 93.3% purity in LCMS at 220 nm) as black brown oil. ¹H NMR: (400 MHz, DMSO-d₆) δ 7.65 (s, 1H), 7.38-7.36 (m, 1H), 7.26-7.25 (m, 1H), 7.09-7.06 (m, 1H), 3.85 (m, 3H). (ESI⁺) m/z: 305.7 (M+H)⁺, (C₁₁H₇D₃BrClN₂O).

B. 3-(5-(5-(2-Chloro-4-methoxyphenyl)-1-(methyl-d₃)-1H-pyrazol-4-yl)-1-oxoisoindolin-2-yl)piperidine-2,6-dione: To a solution of 4-bromo-5-(2-chloro-4-methoxyphenyl)-1-(methyl-d₃)-1H-pyrazole (100 mg, 328 μmol, 1.00 eq), 3-(1-oxo-5-(4,4,5,5-tetramethyl-1,3,2-dioxaborolan-2-yl)isoindolin-2-yl)piperidine-2,6-dione (425 mg, 1.15 mmol, 3.50 eq) and K₃PO₄ (209 mg, 984 μmol, 3.00 eq) in dioxane (5.00 mL) and H₂O (0.25 mL) was added Ru-Phos-Pd-G₃ (54.9 mg, 65.6 μmol, 0.20 eq) under N₂. The mixture was stirred at 100° C. for 6 h under N₂. The reaction mixture was concentrated under reduced pressure to get a residue at 45° C. The residue was purified by preparative-HPLC (using a Phenomenex luna C18 (150×25 mm×5 μm) and gradient of 29.0%-54.0% acetonitrile in water containing 0.05% FA over 3 min at a flow rate of 25 mL/min) to give the title compound (17.1 mg, 36.3 μmol, 11.0% yield, 99.2% purity in HPLC at 220 nm) as white solid. ¹H NMR: (400 MHz, DMSO-d₆) δ 10.9 (s, 1H), 7.98-7.97 (m, 1H), 7.56-7.55 (m, 1H), 7.39-7.36 (m, 2H), 7.28-7.28 (m, 1H), 7.15-7.09 (m, 1H), 7.08-7.06 (m, 1H), 5.09-5.04 (m, 1H), 4.38-4.18 (m, 2H), 3.86 (s, 3H), 2.90-2.86 (m, 1H), 2.59-2.55 (m, 1H), 2.38-2.32 (m, 1H), 1.98-1.96 (m, 1H). Deuterated ratio: 98.4%. (ESI⁺) m/z: 468.2 (M+H)⁺, (C₂₄H₁₈D₃ClN₄O₄).

### Example 435

### Synthesis of 3-(5-(1-(methyl-d₃)-5-(4-(trifluoromethoxy)phenyl)-1H-pyrazol-4-yl)-1-oxoisoindolin-2-yl) piperidine-2,6-dione

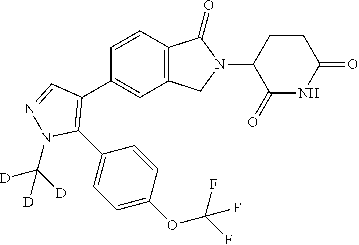 [SMILES: [2H]C([2H])([2H])n1ncc(-c2ccc3c(c2)CN(C2CCC(=O)NC2=O)C3=O)c1-c1ccc(OC(F)(F)F)cc1]

A. 4-Bromo-1-(methyl-d₃)-5-(4-(trifluoromethoxy)phenyl)-1H-pyrazole: To a solution of 4-bromo-1-(trideuteriomethyl)pyrazole (1.00 g, 6.10 mmol, 1.00 eq) and 1-bromo-4-(trifluoromethoxy)benzene (4.41 g, 18.2 mmol, 3.00 eq) in DMF (20 mL) was added Pd(OAc)₂ (136 mg, 0.61 mmol, 0.10 eq), K₂CO₃ (1.68 g, 12.2 mmol, 2.00 eq) and P(oxole)₃ (283 mg, 1.22 mmol, 0.20 eq) under N₂. The mixture was stirred at 100° C. for 12 h under N₂. After the reaction was completed, the reaction mixture was filtered and the filtrate was poured into 50 mL of water and extracted with EtOAc (3×50.0 mL). The combined organic layers were washed with brine (3×10.0 mL), dried over Na₂SO₄ and concentrated in vacuum to get a residue. The residue was purified by column chromatography (SiO₂, Petroleum ether:EtOAc=10:1 to 5:1, R_{f}=0.50 (Petroleum ether:EtOAc=5:1)) to give the title compound (800 mg, 1.99 mmol, 32.3% yield, 80.5% purity by LCMS at 220 nm) as yellow oil. (ESI⁺) m/z: 323.9 (M+H)⁺, (C₁₁H₅D₃F₃BrN₂O).

B. 3-(5-(1-(Methyl-d₃)-5-(4-(trifluoromethoxy)phenyl)-1H-pyrazol-4-yl)-1-oxoisoindolin-2-yl)piperidine-2,6-dione: To a solution of 4-bromo-1-(trideuteriomethyl)-5-[4-(trifluoromethoxy)phenyl]pyrazole (100 mg, 248 μmol, 1.00 eq) in dioxane (4.00 mL) and H₂O (0.20 mL) was added 3-[1-oxo-5-(4,4,5,5-tetramethyl-1,3,2-dioxaborolan-2-yl)isoindolin-2-yl]piperidine-2,6-dione (229 mg, 620 μmol, 2.50 eq), K₃PO₄ (105 mg, 496 μmol, 2.00 eq) and Ru-Phos-Pd-G₃ (20.7 mg, 24.8 μmol, 0.10 eq) under N₂. The mixture was stirred at 100° C. for 2 h under N₂. After the reaction was completed, the reaction mixture was filtered and the filtrate was concentrated under vacuum to give a residue. The residue was purified by preparative-TLC (Dichloromethane:Methanol=10:1, R_{f}=0.60) and by preparative-HPLC (using a Welch Xtimate C18 (150 mm×25 mm 5 μm) and gradient of 26-56% acetonitrile in water containing 0.5% TFA over 10 min at a flow rate of 25.0 mL/min) to give the title compound (20.0 mg, 40.7 μmol, 16.3% yield, 99.2% purity by HPLC at 220 nm) as a white solid. ¹H NMR: (400 MHz, DMSO-d₆) δ 10.9 (s, 1H), 7.90 (s, 1H), 7.60-7.49 (m, 5H), 7.37 (s, 1H), 7.20 (d, J=8.8 Hz, 1H), 5.07 (dd, J=13.2, 4.8 Hz, 1H), 4.28 (dd, J=55.6, 17.2 Hz, 2H), 2.95-2.85 (m, 1H), 2.60-2.59 (m, 1H), 2.40-2.35 (m, 1H), 2.00-1.94 (m, 1H). (ESI⁺) m/z: 488.0 (M+H)⁺, (C₂₄H₁₆D₃F₃N₄O₄).

### Example 436

### Synthesis of 3-(5-(5-(4-(difluoromethoxy)phenyl)-1-(methyl-d₃)-1H-pyrazol-4-yl)-1-oxoisoindolin-2-yl)piperidine-2,6-dione

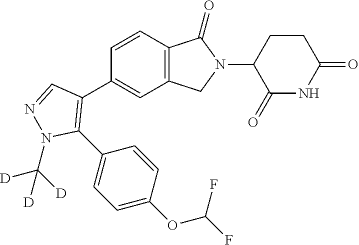 [SMILES: [2H]C([2H])([2H])n1ncc(-c2ccc3c(c2)CN(C2CCC(=O)NC2=O)C3=O)c1-c1ccc(OC(F)F)cc1]

A. 4-Iodo-1-(methyl-d₃)-5-(4-(trifluoromethoxy)phenyl)-1H-pyrazole: To a solution of 4-iodo-1-(trideuteriomethyl)pyrazole (1.00 g, 4.80 mmol, 1.00 eq) and 1-bromo-4-(difluoromethoxy)benzene (3.16 g, 14.1 mmol, 1.93 mL, 3.00 eq) in DMF (20.0 mL) was added Pd(OAc)₂ (105 mg, 472 μmol, 0.10 eq), P(oxole)₃ (218 mg, 944 μmol, 0.20 eq) and K₂CO₃ (1.30 g, 9.44 mmol, 2.00 eq). The mixture was stirred at 100° C. for 12 h under N₂. After the reaction was completed, the reaction mixture was filtered and the filtrate was poured into 50 mL of water and extracted with EtOAc (3×50.0 mL). The combined organic layers were washed with brine (3×10.0 mL), dried over Na₂SO₄ and concentrated in vacuum to get a residue. The residue was purified by column chromatography (SiO₂, Petroleum ether:EtOAc=10:1 to 3:1, R_{f}=0.40) to give the title compound (180 mg, 472 μmol, 10.0% yield, 92.7% purity by LCMS at 220 nm) as a brown solid. (ESI⁺) m/z: 353.9 (M+H)⁺, (C₁₁H₆D₃F₂IN₂O).

B. 3-(5-(5-(4-(difluoromethoxy)phenyl)-1-(methyl-d₃)-1H-pyrazol-4-yl)-1-oxoisoindolin-2-yl)piperidine-2,6-dione: To a solution of 5-[4-(difluoromethoxy)phenyl]-4-iodo-1-(trideuteriomethyl)pyrazole (180 mg, 472 μmol, 1.00 eq) in dioxane (2.00 mL) and H₂O (0.10 mL) was added 3-[1-oxo-5-(4,4,5,5-tetramethyl-1,3,2-dioxaborolan-2-yl)isoindolin-2-yl]piperidine-2,6-dione (437 mg, 1.18 mmol, 2.5 eq), RuPhos-Pd-G₃ (39.5 mg, 47.2 μmol, 0.10 eq) and K₃PO₄ (200 mg, 945 μmol, 2.00 eq). The mixture was stirred at 100° C. for 2 h under N₂. After the reaction was completed, the reaction mixture was filtered and the filtrate was concentrated under vacuum to give a residue. The residue was purified by Pre-TLC (Dichloromethane:Methanol=10:1, R_{f}=0.60) and by prep-HPLC (column: Welch Xtimate C18 150*25 mm*5 um; mobile phase: [water(TFA)-ACN]; gradient: 26%-56% B over min). to give the title compound (20.0 mg, 42.5 μmol, 30.1% yield, 99.8% purity by HPLC at 220 nm) as a white solid. ¹H NMR: (400 MHz, DMSO-d₆) δ 10.9 (s, 1H), 7.89 (s, 1H), 7.57 (d, J=8.0 Hz, 1H), 7.48-7.44 (m, 2H), 7.39 (s, 1H), 7.36 (t, J=74.0 Hz, 1H), 7.33-7.28 (m, 2H), 7.22 (d, J=8.0 Hz, 1H), 5.07 (dd, J=13.2, 5.2 Hz, 1H), 4.29 (dd, J=56.0, 17.6 Hz, 2H), 2.95-2.84 (m, 1H), 2.64-2.54 (m, 1H), 2.43-2.33 (m, 1H), 2.01-1.94 (m, 1H). (ESI⁺) m/z: 470.2 (M+H)⁺, (C₂₄H₁₇D₃F₂N₄O₄).

### Example 437

### Synthesis of 3-(5-(1-(methyl-d₃)-2-phenyl-1H-imidazol-4-yl)-1-oxoisoindolin-2-yl)piperidine-2,6-dione

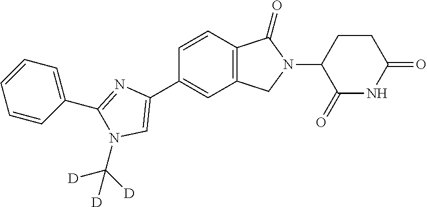 [SMILES: [2H]C([2H])([2H])n1cc(-c2ccc3c(c2)CN(C2CCC(=O)NC2=O)C3=O)nc1-c1ccccc1]

A. 4-Bromo-1-(methyl-d₃)-2-phenyl-1H-imidazole: To a solution of NaH (80.6 mg, 2.02 mmol, 60% purity, 1.50 eq) in THF (10.0 mL) was added 4-bromo-2-phenyl-1H-imidazole (0.30 g, 1.34 mmol, 1.00 eq) at 0° C. under N₂, the reaction mixture was stirred at 0° C. for 30 min under N₂. To the reaction mixture was added CD₃I (51.2 mg, 2.69 mmol, 2.00 eq) at 0° C. The reaction mixture was stirred at 25° C. for 2 h under N₂. The reaction mixture was poured into 50.0 mL H₂O under N₂, extracted with ethyl acetate (3×50.0 mL), washed with brine (50.0 mL), dried over Na₂SO₄ and concentrated to get a residue. The crude product was purified by preparative-TLC (Methanol:Dichloromethane=100:1, R_{f}=0.20) to give the title compound (0.14 g, 583 μmol, 43.3% yield). ¹H NMR: (400 MHz, CDCl₃) δ 7.62-7.60 (m, 2H), 7.47-7.42 (m, 3H), 6.94 (s, 1H). (ESI⁺) m/z: 240.1 (M+H)⁺, (C₁₀H₆D₃BrN₂).

B. 3-(5-(1-(methyl-d₃)-2-phenyl-1H-imidazol-4-yl)-1-oxoisoindolin-2-yl)piperidine-2,6-dione: To a solution of 4-bromo-2-phenyl-1-(trideuteriomethyl)imidazole (0.10 g, 416 μmol, 1.00 eq), 3-[1-oxo-5-(4,4,5,5-tetramethyl-1,3,2-dioxaborolan-2-yl)isoindolin-2-yl]piperidine-2,6-dione (308 mg, 832 μmol, 2.00 eq) in dioxane (3.00 mL) and H₂O (0.15 mL) was added K₃PO₄ (176 mg, 832 μmol, 2.00 eq) and Ru-Phos-Pd-G₃ (34.8 mg, 41.6 μmol, 0.10 eq) under N₂. The reaction mixture was stirred at 100° C. for 2 h under N₂. The reaction mixture was concentrated in vacuum to give residue. The residue was purified by preparative-HPLC (using a Welch Xtimate C18 (150 mm×25 mm 5 μm) and gradient of 1-31% acetonitrile in water containing 0.05% FA over 10 min at a flow rate of 25.0 mL/min) to give the title compound (43.6 mg, 107 μmol, 25.8% yield, 99.4% purity in HPLC at 220 nm) as white solid. ¹H NMR: (400 MHz, DMSO-d₆) δ 11.0 (s, 1H), 8.21 (s, 1H), 8.06 (s, 1H), 7.97 (d, J=7.6 Hz, 1H), 7.83-7.80 (m, 3H), 7.62 (s, 3H), 5.16-5.11 (m, 1H), 4.55-4.38 (m, 2H), 2.98-2.89 (m, 1H), 2.63-2.62 (m, 1H), 2.45-2.40 (m, 1H), 2.04-2.02 (m, 1H). (ESI⁺) m/z: 404.2 (M+H)⁺, (C₂₃H₁₇D₃N₄O₃).

### Example 438

The compound of Example 438 was prepared according to the procedure of Scheme 1 as shown in Examples 55-116.

| | | MW + 1 | Step 1 |
|---|---|---|---|
| Example | Structure | (Observed) | Conditions |
| 438 | 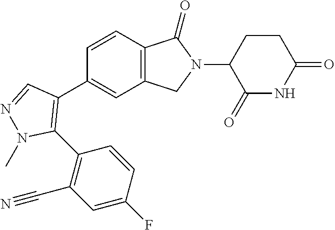 [SMILES: Cn1ncc(-c2ccc3c(c2)CN(C2CCC(=O)NC2=O)C3=O)c1-c1ccc(F)cc1C#N] | 444.2 | 2 |

### Examples 439-449

The compounds of Example 439-449 were prepared according to the procedure of Scheme 2 as shown in Examples 161-204.

| | | MW + |
|---|---|---|
| Exam- | | 1 (Ob- |
| ple | Structure | served) |
| 439 | 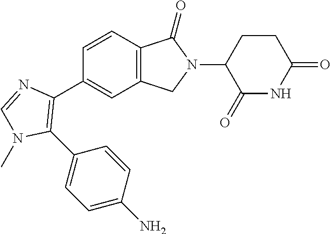 [SMILES: Cn1cnc(-c2ccc3c(c2)CN(C2CCC(=O)NC2=O)C3=O)c1-c1ccc(N)cc1] | 416.2 |
| 440 | 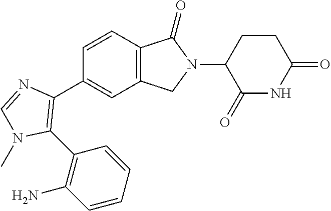 [SMILES: Cn1cnc(-c2ccc3c(c2)CN(C2CCC(=O)NC2=O)C3=O)c1-c1ccccc1N] | 416.2 |
| 441 | 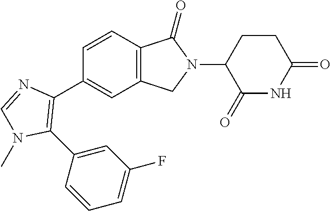 [SMILES: Cn1cnc(-c2ccc3c(c2)CN(C2CCC(=O)NC2=O)C3=O)c1-c1cccc(F)c1] | 419.2 |
| 442 | 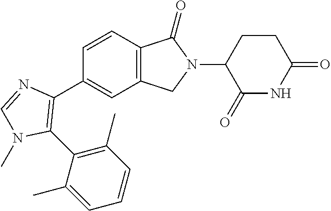 [SMILES: Cc1cccc(C)c1-c1c(-c2ccc3c(c2)CN(C2CCC(=O)NC2=O)C3=O)ncn1C] | 429.3 |
| 443 | 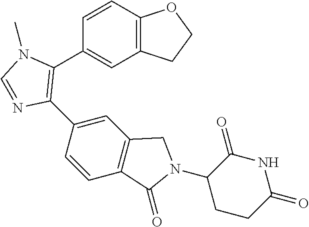 [SMILES: Cn1cnc(-c2ccc3c(c2)CN(C2CCC(=O)NC2=O)C3=O)c1-c1ccc2c(c1)CCO2] | 443.2 |
| 444 | 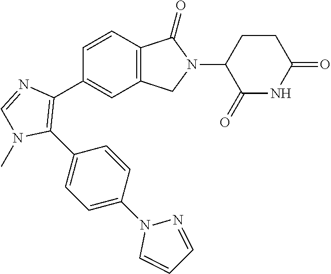 [SMILES: Cn1cnc(-c2ccc3c(c2)CN(C2CCC(=O)NC2=O)C3=O)c1-c1ccc(-n2cccn2)cc1] | 467.3 |
| 445 | 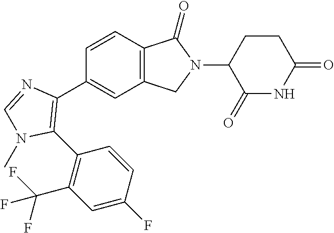 [SMILES: Cn1cnc(-c2ccc3c(c2)CN(C2CCC(=O)NC2=O)C3=O)c1-c1ccc(F)cc1C(F)(F)F] | 487.3 |
| 446 | 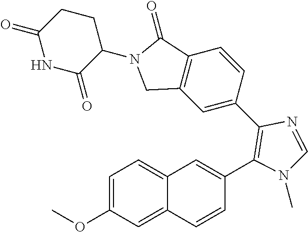 [SMILES: COc1ccc2cc(-c3c(-c4ccc5c(c4)CN(C4CCC(=O)NC4=O)C5=O)ncn3C)ccc2c1] | 481.3 |
| 447 | 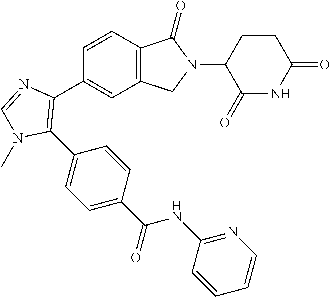 [SMILES: Cn1cnc(-c2ccc3c(c2)CN(C2CCC(=O)NC2=O)C3=O)c1-c1ccc(C(=O)Nc2ccccn2)cc1] | 521.3 |
| 448 | 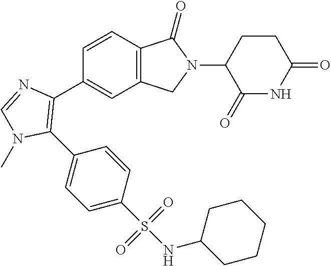 [SMILES: Cn1cnc(-c2ccc3c(c2)CN(C2CCC(=O)NC2=O)C3=O)c1-c1ccc(S(=O)(=O)NC2CCCCC2)cc1] | 562.3 |
| 449 | 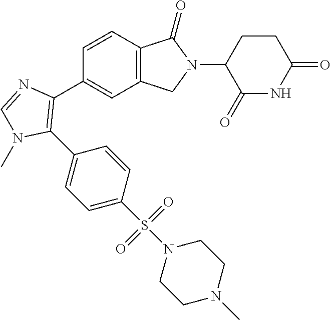 [SMILES: CN1CCN(S(=O)(=O)c2ccc(-c3c(-c4ccc5c(c4)CN(C4CCC(=O)NC4=O)C5=O)ncn3C)cc2)CC1] | 563.3 |

### Examples 450-481

| | | MW + |
|---|---|---|
| Exam- | | 1 (Ob- |
| ple | Structure | served) |
| 450 | 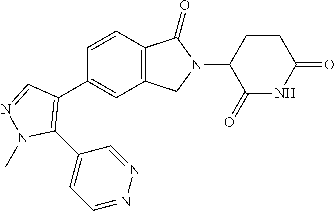 [SMILES: Cn1ncc(-c2ccc3c(c2)CN(C2CCC(=O)NC2=O)C3=O)c1-c1ccnnc1] | 403.3 |
| 451 | 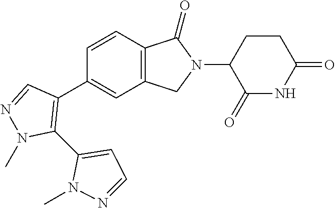 [SMILES: Cn1nccc1-c1c(-c2ccc3c(c2)CN(C2CCC(=O)NC2=O)C3=O)cnn1C] | 405.2 |
| 452 | 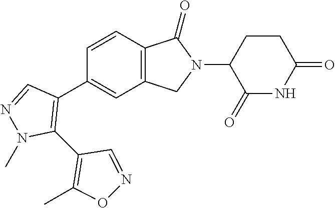 [SMILES: Cc1oncc1-c1c(-c2ccc3c(c2)CN(C2CCC(=O)NC2=O)C3=O)cnn1C] | 406.2 |
| 452 | 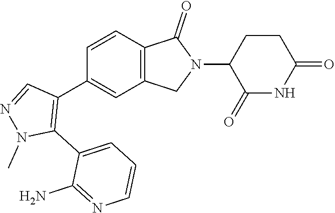 [SMILES: Cn1ncc(-c2ccc3c(c2)CN(C2CCC(=O)NC2=O)C3=O)c1-c1cccnc1N] | 417.2 |
| 454 | 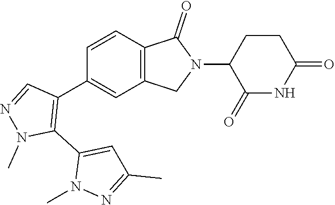 [SMILES: Cc1cc(-c2c(-c3ccc4c(c3)CN(C3CCC(=O)NC3=O)C4=O)cnn2C)n(C)n1] | 419.2 |
| 455 | 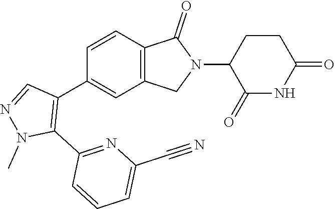 [SMILES: Cn1ncc(-c2ccc3c(c2)CN(C2CCC(=O)NC2=O)C3=O)c1-c1cccc(C#N)n1] | 427.2 |
| 456 | 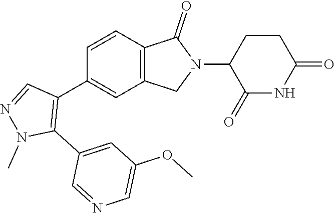 [SMILES: COc1cncc(-c2c(-c3ccc4c(c3)CN(C3CCC(=O)NC3=O)C4=O)cnn2C)c1] | 432.2 |
| 457 | 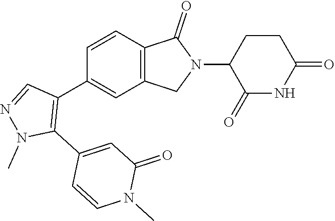 [SMILES: Cn1ncc(-c2ccc3c(c2)CN(C2CCC(=O)NC2=O)C3=O)c1-c1ccn(C)c(=O)c1] | 432.3 |
| 458 | 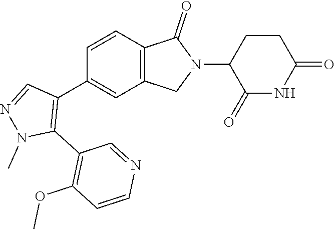 [SMILES: COc1ccncc1-c1c(-c2ccc3c(c2)CN(C2CCC(=O)NC2=O)C3=O)cnn1C] | 432.3 |
| 459 | 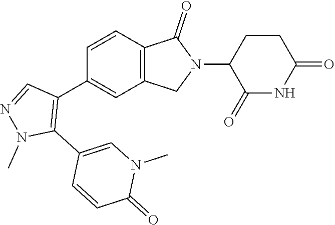 [SMILES: Cn1ncc(-c2ccc3c(c2)CN(C2CCC(=O)NC2=O)C3=O)c1-c1ccc(=O)n(C)c1] | 432.2 |
| 460 | 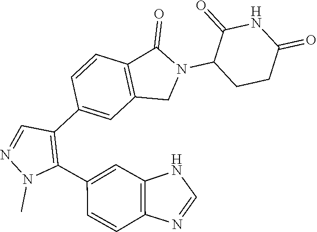 [SMILES: Cn1ncc(-c2ccc3c(c2)CN(C2CCC(=O)NC2=O)C3=O)c1-c1ccc2nc[nH]c2c1] | 441.3 |
| 461 | 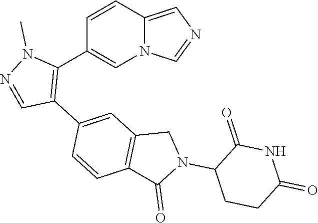 [SMILES: Cn1ncc(-c2ccc3c(c2)CN(C2CCC(=O)NC2=O)C3=O)c1-c1ccc2cncn2c1] | 441.2 |
| 462 | 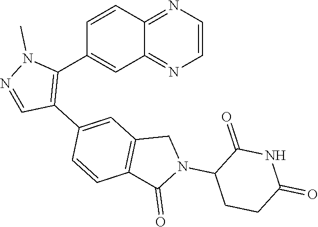 [SMILES: Cn1ncc(-c2ccc3c(c2)CN(C2CCC(=O)NC2=O)C3=O)c1-c1ccc2nccnc2c1] | 453.3 |
| 463 | 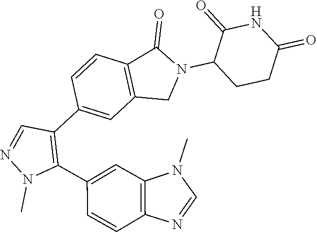 [SMILES: Cn1ncc(-c2ccc3c(c2)CN(C2CCC(=O)NC2=O)C3=O)c1-c1ccc2ncn(C)c2c1] | 455.3 |
| 464 | 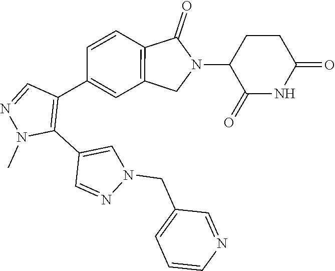 [SMILES: Cn1ncc(-c2ccc3c(c2)CN(C2CCC(=O)NC2=O)C3=O)c1-c1cnn(Cc2cccnc2)c1] | 482.3 |
| 465 | 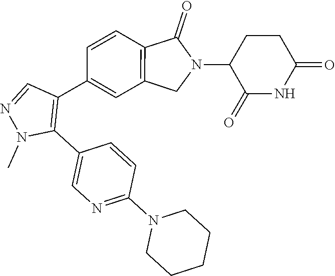 [SMILES: Cn1ncc(-c2ccc3c(c2)CN(C2CCC(=O)NC2=O)C3=O)c1-c1ccc(N2CCCCC2)nc1] | 485.3 |
| 466 | 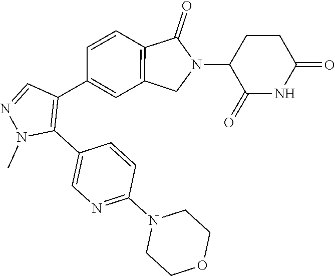 [SMILES: Cn1ncc(-c2ccc3c(c2)CN(C2CCC(=O)NC2=O)C3=O)c1-c1ccc(N2CCOCC2)nc1] | 487.3 |
| 467 | 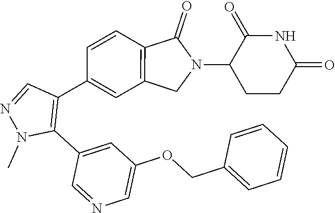 [SMILES: Cn1ncc(-c2ccc3c(c2)CN(C2CCC(=O)NC2=O)C3=O)c1-c1cncc(OCc2ccccc2)c1] | 508.3 |
| 468 | 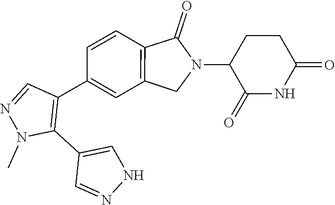 [SMILES: Cn1ncc(-c2ccc3c(c2)CN(C2CCC(=O)NC2=O)C3=O)c1-c1cn[nH]c1] | 391.2 |
| 469 | 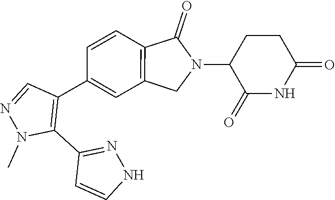 [SMILES: Cn1ncc(-c2ccc3c(c2)CN(C2CCC(=O)NC2=O)C3=O)c1-c1cc[nH]n1] | 391.2 |
| 470 | 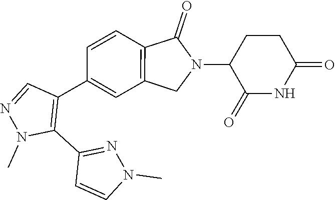 [SMILES: Cn1ccc(-c2c(-c3ccc4c(c3)CN(C3CCC(=O)NC3=O)C4=O)cnn2C)n1] | 405.2 |
| 471 | 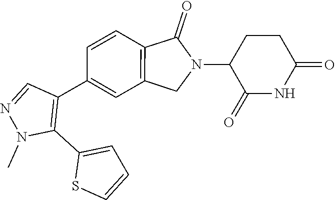 [SMILES: Cn1ncc(-c2ccc3c(c2)CN(C2CCC(=O)NC2=O)C3=O)c1-c1cccs1] | 407.2 |
| 472 | 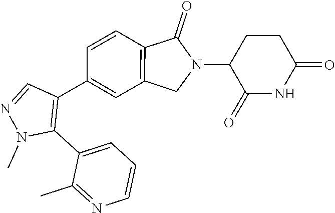 [SMILES: Cc1ncccc1-c1c(-c2ccc3c(c2)CN(C2CCC(=O)NC2=O)C3=O)cnn1C] | 416.2 |
| 473 | 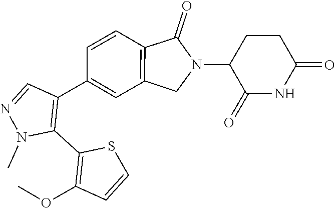 [SMILES: COc1ccsc1-c1c(-c2ccc3c(c2)CN(C2CCC(=O)NC2=O)C3=O)cnn1C] | 437.2 |
| 474 | 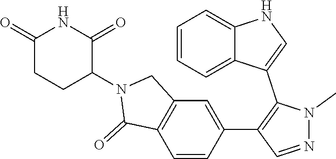 [SMILES: Cn1ncc(-c2ccc3c(c2)CN(C2CCC(=O)NC2=O)C3=O)c1-c1c[nH]c2ccccc12] | 440.3 |
| 475 | 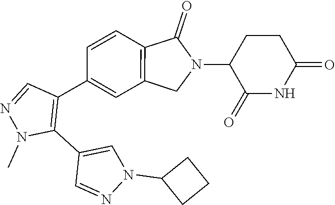 [SMILES: Cn1ncc(-c2ccc3c(c2)CN(C2CCC(=O)NC2=O)C3=O)c1-c1cnn(C2CCC2)c1] | 445.3 |
| 476 | 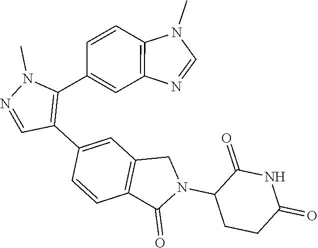 [SMILES: Cn1ncc(-c2ccc3c(c2)CN(C2CCC(=O)NC2=O)C3=O)c1-c1ccc2c(c1)ncn2C] | 455.3 |
| 477 | 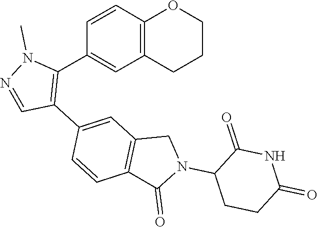 [SMILES: Cn1ncc(-c2ccc3c(c2)CN(C2CCC(=O)NC2=O)C3=O)c1-c1ccc2c(c1)CCCO2] | 457.3 |
| 478 | 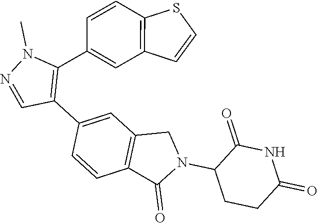 [SMILES: Cn1ncc(-c2ccc3c(c2)CN(C2CCC(=O)NC2=O)C3=O)c1-c1ccc2sccc2c1] | 457.2 |
| 479 | 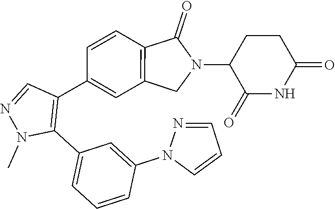 [SMILES: Cn1ncc(-c2ccc3c(c2)CN(C2CCC(=O)NC2=O)C3=O)c1-c1cccc(-n2cccn2)c1] | 467.2 |
| 480 | 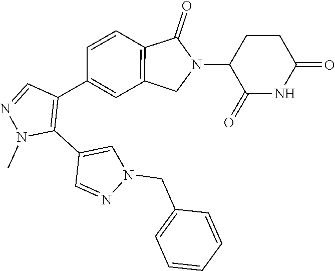 [SMILES: Cn1ncc(-c2ccc3c(c2)CN(C2CCC(=O)NC2=O)C3=O)c1-c1cnn(Cc2ccccc2)c1] | 481.3 |
| 481 | 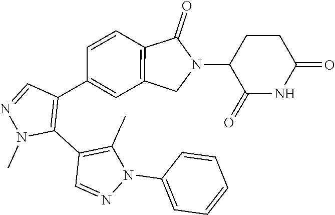 [SMILES: Cc1c(-c2c(-c3ccc4c(c3)CN(C3CCC(=O)NC3=O)C4=O)cnn2C)cnn1-c1ccccc1] | 481.3 |

### Examples 482-487

The compounds of Example 482-487 were prepared according to the procedure of Scheme 3 as shown in Examples 317-332.

| | | MW + |
|---|---|---|
| Exam- | | 1 (Ob- |
| ple | Structure | served) |
| 482 | 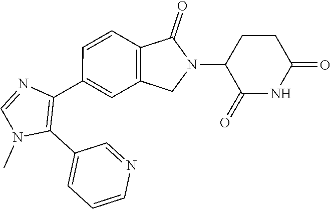 [SMILES: Cn1cnc(-c2ccc3c(c2)CN(C2CCC(=O)NC2=O)C3=O)c1-c1cccnc1] | 402.2 |
| 483 | 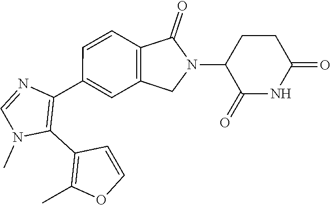 [SMILES: Cc1occc1-c1c(-c2ccc3c(c2)CN(C2CCC(=O)NC2=O)C3=O)ncn1C] | 405.2 |
| 484 | 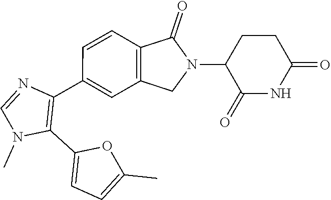 [SMILES: Cc1ccc(-c2c(-c3ccc4c(c3)CN(C3CCC(=O)NC3=O)C4=O)ncn2C)o1] | 405.2 |
| 485 | 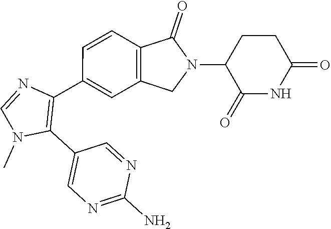 [SMILES: Cn1cnc(-c2ccc3c(c2)CN(C2CCC(=O)NC2=O)C3=O)c1-c1cnc(N)nc1] | 418.3 |
| 486 | 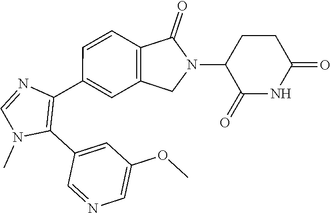 [SMILES: COc1cncc(-c2c(-c3ccc4c(c3)CN(C3CCC(=O)NC3=O)C4=O)ncn2C)c1] | 432.2 |
| 487 | 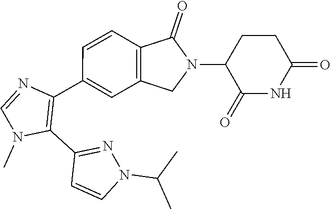 [SMILES: CC(C)n1ccc(-c2c(-c3ccc4c(c3)CN(C3CCC(=O)NC3=O)C4=O)ncn2C)n1] | 433.3 |

### Example 488

### Synthesis of 3-(5-(5-(4-fluorophenyl)-1-methyl-1H-pyrazol-3-yl)-1-oxoisoindolin-2-yl)piperidine-2,6-dione

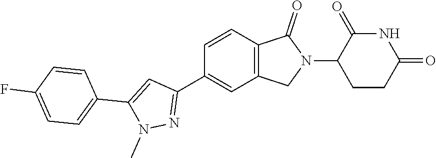 [SMILES: Cn1nc(-c2ccc3c(c2)CN(C2CCC(=O)NC2=O)C3=O)cc1-c1ccc(F)cc1]

A. 3-Bromo-5-(4-fluorophenyl)-1-methyl-1H-pyrazole: To a solution of 3,5-dibromo-1-methyl-1H-pyrazole (500 mg, 2.08 mmol, 1.00 eq), (4-fluorophenyl)boronic acid (376 mg, 2.69 mmol, 1.29 eq) and K₂CO₃ (864 mg, 6.25 mmol, 3.00 eq) in dioxane (8.00 mL) and H₂O (2.00 mL) was added Pd(dppf)Cl₂·CH₂Cl₂ (170 mg, 208 μmol, 0.10 eq) under N₂. Then the mixture was stirred at 80° C. for 10 h under N₂. The mixture was poured into water (30.0 mL) and extracted with ethyl acetate (3×30 mL) to collect the organic layer, the combined organic layers were dried over by Na₂SO₄, filtered and the filtrate was concentrated under vacuum to get a residue. The residue was purified by column chromatography (SiO₂, Ethyl acetate:Petroleum ether=50:1 to 3:1, TLC: Ethyl acetate:Petroleum ether=8:1 R_{f}=0.20) to give the title compound (150 mg, 582 μmol, 14.0% yield, 98.9% purity in LCMS at 220 nm) was obtained as white solid. ¹H NMR: (400 MHz, DMSO-d₆) δ 7.39-7.36 (m, 2H), 7.27 (s, 1H), 7.20-7.15 (m, 2H), 3.83 (s, 3H), (ESI⁺) m/z: 271.1 (M+H)⁺, (C₁₀H₈BrFN₂).

B. 3-(5-(5-(4-Fluorophenyl)-1-methyl-1H-pyrazol-3-yl)-1-oxoisoindolin-2-yl) piperidine-2, 6-dione: To a solution of 3-bromo-5-(4-fluorophenyl)-1-methyl-1H-pyrazole (115 mg, 451 μmol, 1.00 eq), 3-(1-oxo-5-(4,4,5,5-tetramethyl-1,3,2-dioxaborolan-2-yl)isoindolin-2-yl)piperidine-2,6-dione (500 mg, 1.35 mmol, 3.00 eq) and K₃PO₄ (287 mg, 1.35 mmol, 3.00 eq) in dioxane (5.00 mL) and H₂O (0.25 mL) was added Ru-Phos-Pd-G₃ (75.4 mg, 90.2 μmol, 0.20 eq) under N₂. The mixture was stirred at 100° C. for 2 h under N₂. The mixture was filtered to collect liquid and concentrated under reduced pressure to get residue. The residue was purified by preparative-HPLC (using a Phenomenex luna C18 (150×25 mm×10 μm) and gradient of 24%-54% acetonitrile in water containing 0.50% TFA over 15 min at a flow rate of 25 mL/min) to give the title compound (26.1 mg, 62.2 μmol, 13.8% yield, 99.7% purity in HPLC at 220 nm) was obtained as white solid. ¹H NMR: (400 MHz, DMSO-d₆) δ 11.0 (s, 1H), 8.07 (s, 1H), 8.00-7.98 (m, 1H), 7.77-7.75 (m, 1H), 7.69-7.65 (m, 2H), 7.41-7.36 (m, 2H), 7.03 (s, 1H), 5.16-5.11 (m, 1H), 4.53-4.36 (m, 2H), 3.91 (s, 3H), 2.93-2.87 (m, 1H), 2.63-2.52 (m, 1H), 2.47-2.42 (m, 1H), 2.02-1.99 (m, 1H). (ESI⁺) m/z: 419.1 (M+H)⁺, (C₂₃H₁₉FN₄O₃).

### Examples 489-494

The compounds of Example 489-494 were prepared according to the procedure of Scheme 3 as shown in Examples 317-332.

| | | MW + |
|---|---|---|
| Exam- | | 1 (Ob- |
| ple | Structure | served) |
| 489 | 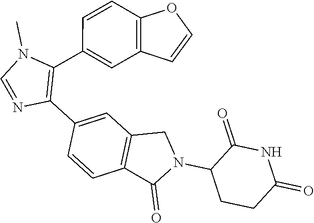 [SMILES: Cn1cnc(-c2ccc3c(c2)CN(C2CCC(=O)NC2=O)C3=O)c1-c1ccc2occc2c1] | 441.3 |
| 490 | 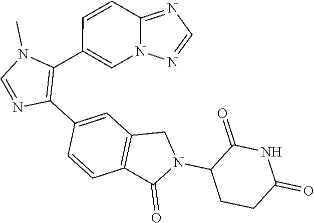 [SMILES: Cn1cnc(-c2ccc3c(c2)CN(C2CCC(=O)NC2=O)C3=O)c1-c1ccc2ncnn2c1] | 442.3 |
| 491 | 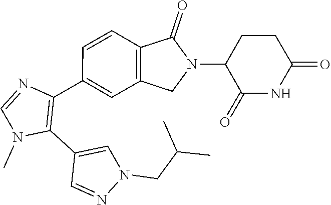 [SMILES: CC(C)Cn1cc(-c2c(-c3ccc4c(c3)CN(C3CCC(=O)NC3=O)C4=O)ncn2C)cn1] | 447.3 |
| 492 | 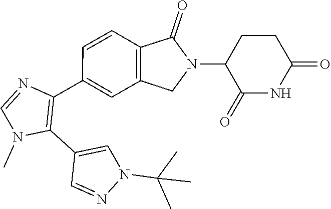 [SMILES: Cn1cnc(-c2ccc3c(c2)CN(C2CCC(=O)NC2=O)C3=O)c1-c1cnn(C(C)(C)C)c1] | 447.3 |
| 493 | 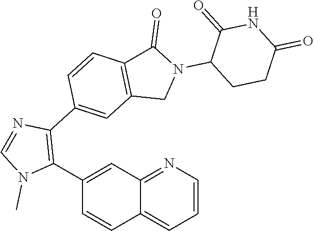 [SMILES: Cn1cnc(-c2ccc3c(c2)CN(C2CCC(=O)NC2=O)C3=O)c1-c1ccc2cccnc2c1] | 452.3 |
| 494 | 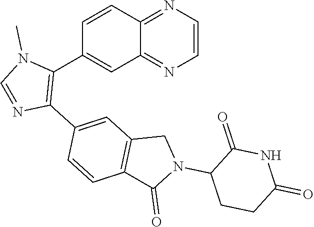 [SMILES: Cn1cnc(-c2ccc3c(c2)CN(C2CCC(=O)NC2=O)C3=O)c1-c1ccc2nccnc2c1] | 453.2 |

### Example 495

### Synthesis of 3-(5-(1-methyl-5-(4-(trifluoromethyl)phenyl)-1H-pyrazol-3-yl)-1-oxoisoindolin-2-yl)piperidine-2,6-dione

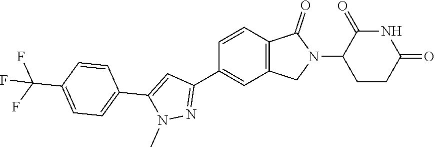 [SMILES: Cn1nc(-c2ccc3c(c2)CN(C2CCC(=O)NC2=O)C3=O)cc1-c1ccc(C(F)(F)F)cc1]

A. 3-Oxo-3-(4-(trifluoromethyl)phenyl)propanenitrile: To a solution of methyl 4-(trifluoromethyl)benzoate (50.0 g, 244 mmol, 39.4 mL, 1.00 eq) and acetonitrile (12.0 g, 293 mmol, 15.4 mL, 1.20 eq) in THF (750 mL) was added NaH (11.7 g, 293 mmol, 60% purity, 1.20 eq) in batches at 0° C. under N₂. Then the mixture was stirred at 70° C. for 2 h under N₂. Then the reaction mixture was cooled down to 0° C. and quenched with 2 M HCl (300 mL) until pH=7. Then the mixture was extracted with EtOAc (3×1000 mL). The combined organic layers were washed with brine (200 mL), dried over Na₂SO₄ and filtered. The filtrate was concentrated in vacuum to get a residue. The residue was purified by column chromatography (SiO₂, Petroleum ether:Ethyl acetate=100:0 to 5:1, R_{f}=0.40 (Petroleum ether:Ethyl acetate=1:1)) to give the title compound (21.0 g, 98.3 mmol, 40.1% yield, 99.8% purity in LCMS at 220 nm) as brown oil. ¹H NMR: (400 MHz, CDCl₃) δ 8.05 (d, J=8.0 Hz, 2H), 7.80 (d, J=8.4 Hz, 2H), 4.14 (s, 2H). (ESI⁺) m/z: 212.0 (M+H)⁺, (C₁₀H₆F₃NO).

B. 5-(4-(Trifluoromethyl)phenyl)-1H-pyrazol-3-amine: To a solution of 3-oxo-3-[4-(trifluoromethyl)phenyl]propanenitrile (21.0 g, 98.3 mmol, 1.00 eq) in EtOH (46.2 mL) was added N₂H₄·H₂O (6.69 g, 130 mmol, 6.48 mL, 98% purity, 1.33 eq) under N₂. The mixture was stirred at 25° C. for 1 h and then heated to 80° C. for additional 1 h. Then the reaction mixture was cooled down to 25° C. and cold water (44.0 ml) was added until the solid was precipitated. The solid was collected and washed with cold water (2×50.0 ml) and dried in vacuum to give the title compound (15.0 g, 63.7 mmol, 64.8% yield, 96.5% purity in LCMS at 220 nm) as a yellow solid without further purification. (ESI⁺) m/z: 227.9 (M+H)⁺, (C₁₀H₈F₃N₃).

C. 3-Iodo-5-(4-(trifluoromethyl)phenyl)-1H-pyrazole: To a solution of 5-[4-(trifluoromethyl)phenyl]-1H-pyrazol-3-amine (5.00 g, 21.2 mmol, 1.00 eq) and TsOH (9.14 g, 53.1 mmol, 2.50 eq) in ACN (250 mL) and H₂O (40.0 mL) was added a solution of NaNO₂ (3.66 g, 53.1 mmol, 2.50 eq) in H₂O (20.0 mL) at 0° C. under N₂. The mixture was stirred at 0° C. for 0.5 h. Then a solution of NaI (15.9 g, 106 mmol, 5.00 eq) in H₂O (20.0 mL) was added dropwise to the reaction mixture at 0° C. under N₂. Then the reaction mixture was stirred at 25° C. for 2 h. The reaction mixture was diluted with 200 mL of water and extracted with ethyl acetate (3×200 mL). The combined organic layers were washed with brine (100 mL), dried over Na₂SO₄ and filtered. The filtrate was concentrated in vacuum to get a residue. The residue was purified by column chromatography (SiO₂, Petroleum ether:Ethyl acetate=100:1 to 5:1, R_{f}=0.40 (Petroleum ether:Ethyl acetate=5:1)) to give the title compound (1.00 g, 2.85 mmol, 13.4% yield, 96.3% purity in LCMS at 220 nm) as a yellow solid. (ESI⁺) m/z: 338.8 (M+H)⁺, (C₁₀H₆F₃IN₂).

D. 3-Iodo-1-methyl-5-(4-(trifluoromethyl)phenyl)-1H-pyrazole: To a solution of 3-iodo-5-[4-(trifluoromethyl)phenyl]-1H-pyrazole (1.00 g, 2.85 mmol, 1.00 eq) in DMF (20.0 mL) was added K₂CO₃ (787 mg, 5.70 mmol, 2.00 eq) and CH₃I (808 mg, 5.70 mmol, 354 μL, 2.00 eq). The mixture was stirred at 25° C. for 16 h. Then the reaction mixture was filtered and the filtrate was diluted with ethyl acetate (20.0 mL). Then the mixture was washed with brine (3×10.0 mL), dried over Na₂SO₄ and filtered. The filtrate was concentrated in vacuum to get a residue. The residue was purified by preparative-TLC (SiO₂, Petroleum ether:Ethyl acetate=8:1, R_{f}=0.20) to give the title compound (150 mg, 412 μmol, 14.4% yield, 96.8% purity in LCMS at 220 nm) as a yellow solid. ¹H NMR: (400 MHz, DMSO-d₆) δ 7.86 (d, J=8.4 Hz, 2H), 7.77 (d, J=8.0 Hz, 2H), 6.75 (s, 1H), 3.87 (s, 3H). (ESI⁺) m/z: 338.8 (M+H)⁺, (C₁₁H₈F₃IN₂).

E. 3-(5-(1-Methyl-5-(4-(trifluoromethyl)phenyl)-1H-pyrazol-3-yl)-1-oxoisoindolin-2-yl)piperidine-2,6-dione: To a solution of 3-iodo-1-methyl-5-[4-(trifluoromethyl)phenyl]pyrazole (150 mg, 412 μmol, 1.00 eq) and 3-[1-oxo-5-(4,4,5,5-tetramethyl-1,3,2-dioxaborolan-2-yl)isoindolin-2-yl]piperidine-2,6-dione (381 mg, 1.03 mmol, 2.50 eq) in dioxane (4.00 mL) and H₂O (0.20 mL) was added Ru-Phos-Pd-G₃ (34.4 mg, 41.2 μmol, 0.10 eq) and K₃PO₄ (175 mg, 824 μmol, 2.00 eq). The mixture was stirred at 100° C. for 6 h under N₂. The reaction mixture was filtered and the filtrate was concentrated in vacuum to get a residue. The residue was purified by preparative-TLC (SiO₂, Dichloromethane:Methanol=10:1, R_{f}=0.50) and by preparative-HPLC (using a Phenomenex Luna C18 (100 mm×30 mm 5 μm) and gradient of 33-63% acetonitrile in water containing 0.5%0 HCl over 10 min at a flow rate of 25.0 mL/min) and then by preparative-HPLC (using a Phenomenex Luna C18 (150 mm×25 mm 10 μm) and gradient of 37-67% acetonitrile in water containing 0.5% FA over 10 min at a flow rate of 25.0 mL/min) to give the title compound (23.87 mg, 50.8 μmol, 12.3% yield, 99.7% purity in HPLC at 220 nm) as a white solid. ¹H NMR: (400 MHz, DMSO-d₆) δ 11.0 (s, 1H), 8.08 (s, 1H), 8.00 (d, J=7.6 Hz, 1H), 7.89 (dd, J=16.4, 8.4 Hz, 4H), 7.77 (d, J=8.0 Hz, 1H), 7.17 (s, 1H), 5.13 (dd, J=13.6, 5.2 Hz, 1H), 4.45 (dd, J=52.8, 17.6 Hz, 2H), 3.97 (s, 3H), 2.98-2.87 (m, 1H), 2.65-2.57 (m, 1H), 2.46-2.36 (m, 1H), 2.07-1.98 (m, 1H). (ESI⁺) m/z: 469.0 (M+H)⁺, (C₂₄H₁₉F₃N₄O₃).

### Examples 496-517

The compounds of Example 496-517 were prepared according to the procedure of Scheme 3 as shown in Examples 317-332.

| | | MW + 1 |
|---|---|---|
| Example | Structure | (Observed) |
| 496 | 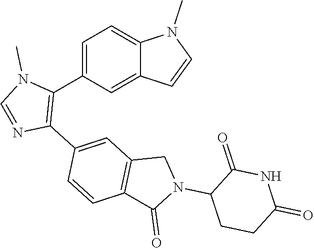 [SMILES: Cn1cnc(-c2ccc3c(c2)CN(C2CCC(=O)NC2=O)C3=O)c1-c1ccc2c(ccn2C)c1] | 454.3 |
| 497 | 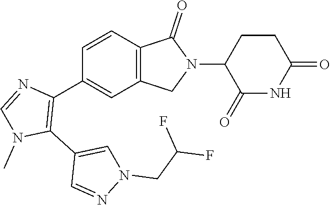 [SMILES: Cn1cnc(-c2ccc3c(c2)CN(C2CCC(=O)NC2=O)C3=O)c1-c1cnn(CC(F)F)c1] | 455.2 |
| 498 | 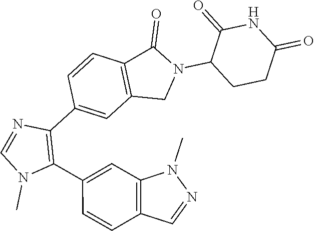 [SMILES: Cn1cnc(-c2ccc3c(c2)CN(C2CCC(=O)NC2=O)C3=O)c1-c1ccc2cnn(C)c2c1] | 455.3 |
| 499 | 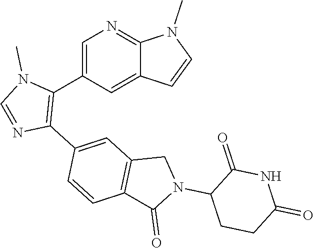 [SMILES: Cn1cnc(-c2ccc3c(c2)CN(C2CCC(=O)NC2=O)C3=O)c1-c1cnc2c(ccn2C)c1] | 455.3 |
| 500 | 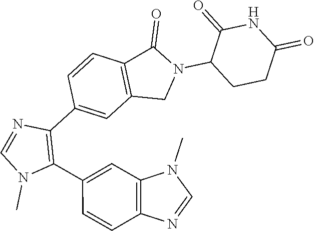 [SMILES: Cn1cnc(-c2ccc3c(c2)CN(C2CCC(=O)NC2=O)C3=O)c1-c1ccc2ncn(C)c2c1] | 455.3 |
| 501 | 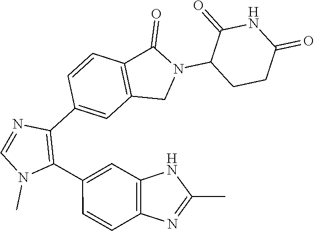 [SMILES: Cc1nc2ccc(-c3c(-c4ccc5c(c4)CN(C4CCC(=O)NC4=O)C5=O)ncn3C)cc2[nH]1] | 455.3 |
| 502 | 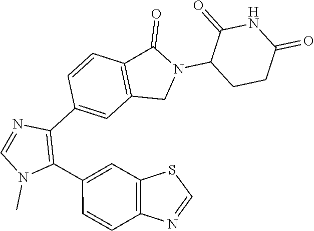 [SMILES: Cn1cnc(-c2ccc3c(c2)CN(C2CCC(=O)NC2=O)C3=O)c1-c1ccc2ncsc2c1] | 458.2 |
| 503 | 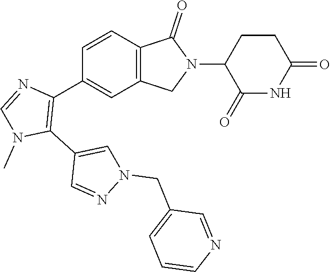 [SMILES: Cn1cnc(-c2ccc3c(c2)CN(C2CCC(=O)NC2=O)C3=O)c1-c1cnn(Cc2cccnc2)c1] | 482.3 |
| 504 | 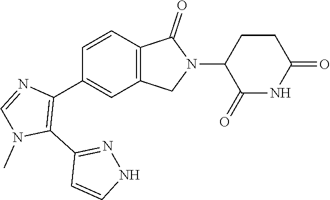 [SMILES: Cn1cnc(-c2ccc3c(c2)CN(C2CCC(=O)NC2=O)C3=O)c1-c1cc[nH]n1] | 391.2 |
| 505 | 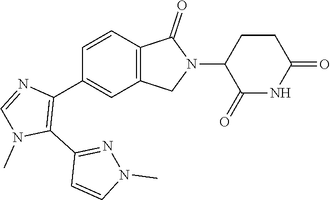 [SMILES: Cn1ccc(-c2c(-c3ccc4c(c3)CN(C3CCC(=O)NC3=O)C4=O)ncn2C)n1] | 405.2 |
| 506 | 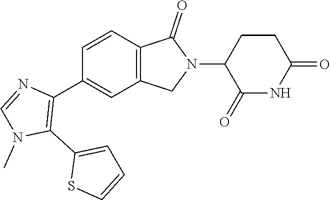 [SMILES: Cn1cnc(-c2ccc3c(c2)CN(C2CCC(=O)NC2=O)C3=O)c1-c1cccs1] | 407.2 |
| 507 | 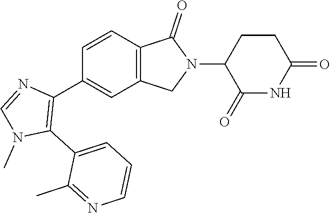 [SMILES: Cc1ncccc1-c1c(-c2ccc3c(c2)CN(C2CCC(=O)NC2=O)C3=O)ncn1C] | 416.2 |
| 508 | 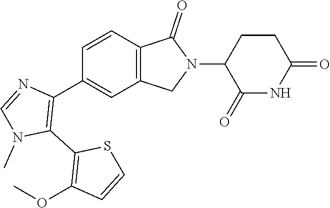 [SMILES: COc1ccsc1-c1c(-c2ccc3c(c2)CN(C2CCC(=O)NC2=O)C3=O)ncn1C] | 437.2 |
| 509 | 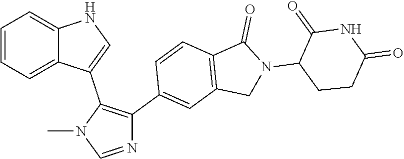 [SMILES: Cn1cnc(-c2ccc3c(c2)CN(C2CCC(=O)NC2=O)C3=O)c1-c1c[nH]c2ccccc12] | 440.2 |
| 510 | 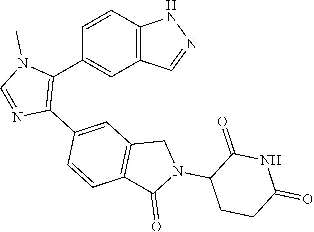 [SMILES: Cn1cnc(-c2ccc3c(c2)CN(C2CCC(=O)NC2=O)C3=O)c1-c1ccc2[nH]ncc2c1] | 441.3 |
| 511 | 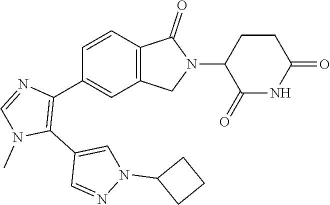 [SMILES: Cn1cnc(-c2ccc3c(c2)CN(C2CCC(=O)NC2=O)C3=O)c1-c1cnn(C2CCC2)c1] | 445.3 |
| 512 | 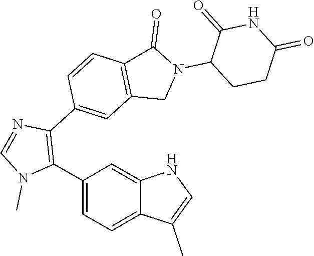 [SMILES: Cc1c[nH]c2cc(-c3c(-c4ccc5c(c4)CN(C4CCC(=O)NC4=O)C5=O)ncn3C)ccc12] | 454.3 |
| 513 | 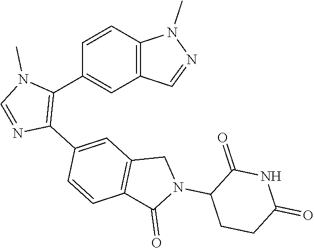 [SMILES: Cn1cnc(-c2ccc3c(c2)CN(C2CCC(=O)NC2=O)C3=O)c1-c1ccc2c(cnn2C)c1] | 455.3 |
| 514 | 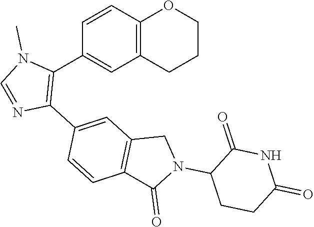 [SMILES: Cn1cnc(-c2ccc3c(c2)CN(C2CCC(=O)NC2=O)C3=O)c1-c1ccc2c(c1)CCCO2] | 457.3 |
| 515 | 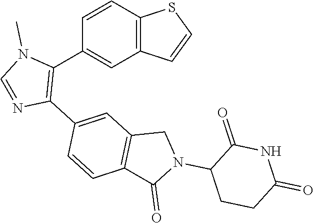 [SMILES: Cn1cnc(-c2ccc3c(c2)CN(C2CCC(=O)NC2=O)C3=O)c1-c1ccc2sccc2c1] | 457.3 |
| 516 | 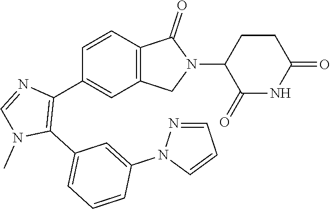 [SMILES: Cn1cnc(-c2ccc3c(c2)CN(C2CCC(=O)NC2=O)C3=O)c1-c1cccc(-n2cccn2)c1] | 467.3 |
| 517 | 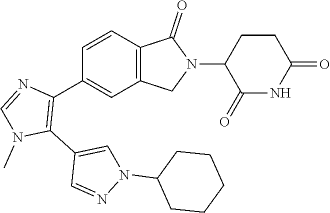 [SMILES: Cn1cnc(-c2ccc3c(c2)CN(C2CCC(=O)NC2=O)C3=O)c1-c1cnn(C2CCCCC2)c1] | 473.3 |

### Example 518

### Synthesis of 3-(5-(5-(2-chloro-4-methoxyphenyl)-1-(difluoromethyl)-1H-pyrazol-4-yl)-1-oxoisoindolin-2-yl)piperidine-2,6-dione

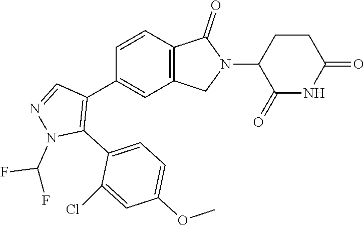 [SMILES: COc1ccc(-c2c(-c3ccc4c(c3)CN(C3CCC(=O)NC3=O)C4=O)cnn2C(F)F)c(Cl)c1]

A. 4-Bromo-5-(2-chloro-4-methoxyphenyl)-1-(difluoromethyl)-1H-pyrazole: To a solution of 4-bromo-1-(difluoromethyl)-1H-pyrazole (300 mg, 1.52 mmol, 1.00 eq) and 1-bromo-2-chloro-4-methoxybenzene (2.02 g, 9.14 mmol, 6.00 eq) in DMF (5.00 mL) was added K₂CO₃ (420 mg, 3.05 mmol, 2.00 eq), P(oxole)₃ (70.0 mg, 304 μmol 1, 0.20 eq) and Pd(OAc)₂ (102 mg, 456 μmol, 0.30 eq) under N₂. The mixture was stirred at 100° C. for 48 h under N₂. The reaction mixture was poured into H₂O (10.0 mL) and extracted with Ethyl acetate (3×30.0 mL). The combined organic layer was washed with brine (3×30.0 mL), and dried over by Na₂SO₄, filtered and concentrate filtrate under reduced pressure to get residue. The residue was purified by prep-TLC (Petroleum ether:Dichloromethane=3:1, R_{f}=0.50) to give the title compound (150 mg, 311 μmol, 20.4% yield, 81.9% purity in LCMS at 220 nm) as light yellow oil. ¹H NMR: (400 MHz, DMSO-d₆) δ 7.31 (s, 1H), 7.17-7.24 (m, 1H), 7.09-6.78 (m, 3H), 3.86 (s, 3H). (ESI⁺) m/z: 336.9 (M+H)⁺, (C₁₁H₈BrClF₂N₂O).

B. 3-(5-(5-(2-chloro-4-methoxyphenyl)-1-(difluoromethyl)-1H-pyrazol-4-yl)-1-oxoisoindolin-2-yl)piperidine-2,6-dione: To a solution of 4-bromo-5-(2-chloro-4-methoxyphenyl)-1-(difluoromethyl)-1H-pyrazole (100 mg, 296 μmol, 1.00 eq), 3-(1-oxo-5-(4,4,5,5-tetramethyl-1,3,2-dioxaborolan-2-yl)isoindolin-2-yl)piperidine-2,6-dione (389 mg, 1.04 mmol, 3.00 eq) and K₃PO₄ (188 mg, 388 μmol, 3.00 eq) in dioxane (5.00 mL) and H₂O (0.25 mL) was added Ru-Phos-Pd-G₃ (49.5 mg, 59.2 μmol, 0.10 eq) under N₂. The mixture was stirred at 100° C. for 5 h under N₂. The reaction mixture was concentrated under reduced pressure to get a residue at 45° C. The residue was purified by preparative-HPLC (using a Phenomenex luna C18 (150×25 mm×5 μm) and gradient of 35.0%-55.0% acetonitrile in water containing 0.05% FA over 10 min at a flow rate of 25 mL/min) to give the title compound (21.7 mg, 39.6 μmol, 18.0% yield, 99.3% purity in HPLC at 220 nm) as white solid. ¹H NMR: (400 MHz, DMSO-d₆) δ 10.9 (s, 1H), 8.35 (s, 1H), 7.62-7.54 (m, 2H), 7.46-7.44 (m, 2H), 7.30-7.27 (m, 2H), 7.10-7.07 (m, 1H), 5.10-5.05 (m, 1H), 4.41-4.22 (m, 2H), 3.88 (s, 3H), 2.89-2.85 (m, 1H), 2.59-2.51 (m, 1H), 2.39-2.35 (m, 1H), 1.98-1.96 (m, 1H). (ESI⁺) m/z: 501.1 (M+H)⁺, (C₂₄H₁₉ClF₂N₄O₄).

### Example 519

### Synthesis of 3-[5-(1-methyl-5-phenyl-1,2,4-triazol-3-yl)-1-oxo-isoindolin-2-yl]piperidine-2,6-dione

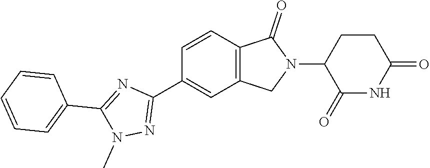 [SMILES: Cn1nc(-c2ccc3c(c2)CN(C2CCC(=O)NC2=O)C3=O)nc1-c1ccccc1]

A. 3-Bromo-1-methyl-5-phenyl-1,2,4-triazole: A solution of 3,5-dibromo-1-methyl-1,2,4-triazole (250 mg, 1.04 mmol, 1.00 eq), tributyl(phenyl)stannane (495 mg, 1.35 mmol, 440 μL, 1.30 eq), LiCl (132 mg, 3.11 mmol, 63.8 μL, 3.00 eq) and Pd(PPh₃)₂Cl₂ (14.5 mg, 20.7 μmol, 0.02 eq) were taken up into a microwave tube in dioxane (2.00 mL). The sealed tube was heated at 110° C. for 15 min under microwave. The residue was purified by column chromatography (SiO₂, Petroleum ether:Ethyl acetate=100:1 to 2:1, R_{f}=0.30) to give the title compound (250 mg, 1.01 mmol, 48.6% yield, 96.2% purity in LCMS at 220 nm) as a white solid. ¹H NMR: (400 MHz, CDCl₃-d₆) δ 7.69-7.66 (m, 2H), 7.54-7.52 (m, 3H), 3.99 (s, 3H). (ESI⁺) m/z: 240.0 (M+H)⁺, (C₉H₈N₃Br).

B. 3-[5-(1-Methyl-5-phenyl-1,2,4-triazol-3-yl)-1-oxo-isoindolin-2-yl]piperidine-2,6-dione: A solution of 3-bromo-1-methyl-5-phenyl-1,2,4-triazole (200 mg, 808 μmol, 1.00 eq), 3-[1-oxo-5-(4,4,5,5-tetramethyl-1,3,2-dioxaborolan-2-yl)isoindolin-2-yl]piperidine-2,6-dione (359 mg, 969 μmol, 1.20 eq), K₃PO₄ (343 mg, 1.62 mmol, 2.00 eq) and RuPhos Pd G₃ (67.5 mg, 80.8 μmol, 0.10 eq) in dioxane (2.00 mL) and H₂O (0.10 mL) was stirred at 100° C. for 2 h. The crude product was purified by preparative-HPLC (using a Welch Ultimate C18 (150 mm×25 mm, 5 m) and gradient of 20-50% acetonitrile in water containing 0.05% FA over 10 min at a flow rate of 25 mL/min) to give the title compound (29.1 mg, 72.1 μmol, 8.93% yield, 99.3% purity in HPLC at 220 nm) as a white solid. ¹H NMR: (400 MHz, DMSO-d₆) δ 11.0 (s, 1H), 8.23-8.19 (m, 2H), 7.88-7.82 (m, 3H), 7.60 (s, 3H), 5.17-5.12 (m, 1H), 4.57-4.40 (m, 2H), 4.05 (s, 3H), 2.95-2.89 (m, 1H), 2.61 (m, 1H), 2.43-2.40 (m, 1H), 2.39-2.02 (m, 1H). (ESI⁺) m/z: 402.1 (M+H)⁺, (C₂₂H₁₉N₅O₃).

### Examples 520-531

The compounds of Example 520-531 were prepared according to the procedure of Scheme 3 as shown in Examples 317-332M

| | | MW + 1 |
|---|---|---|
| Example | Structure | (Observed) |
| 520 | 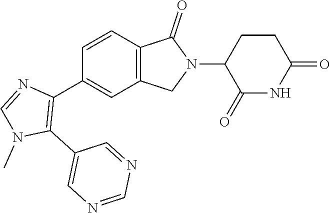 [SMILES: Cn1cnc(-c2ccc3c(c2)CN(C2CCC(=O)NC2=O)C3=O)c1-c1cncnc1] | 403.2 |
| 521 | 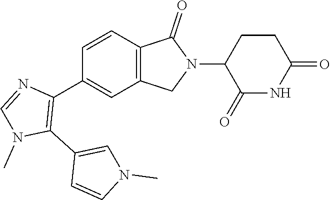 [SMILES: Cn1ccc(-c2c(-c3ccc4c(c3)CN(C3CCC(=O)NC3=O)C4=O)ncn2C)c1] | 404.2 |
| 522 | 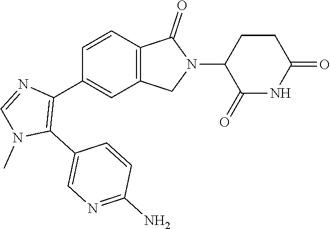 [SMILES: Cn1cnc(-c2ccc3c(c2)CN(C2CCC(=O)NC2=O)C3=O)c1-c1ccc(N)nc1] | 417.3 |
| 523 | 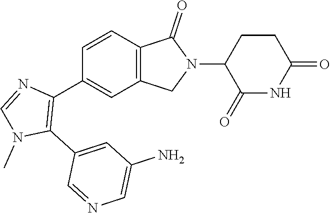 [SMILES: Cn1cnc(-c2ccc3c(c2)CN(C2CCC(=O)NC2=O)C3=O)c1-c1cncc(N)c1] | 417.3 |
| 524 | 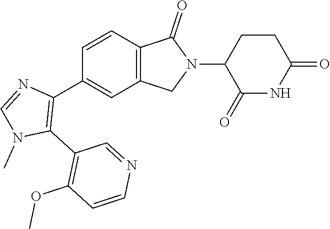 [SMILES: COc1ccncc1-c1c(-c2ccc3c(c2)CN(C2CCC(=O)NC2=O)C3=O)ncn1C] | 432.2 |
| 525 | 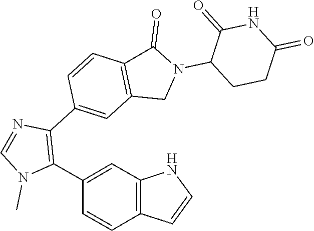 [SMILES: Cn1cnc(-c2ccc3c(c2)CN(C2CCC(=O)NC2=O)C3=O)c1-c1ccc2cc[nH]c2c1] | 440.2 |
| 526 | 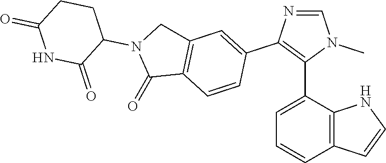 [SMILES: Cn1cnc(-c2ccc3c(c2)CN(C2CCC(=O)NC2=O)C3=O)c1-c1cccc2cc[nH]c12] | 440.3 |
| 527 | 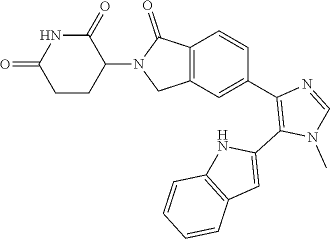 [SMILES: Cn1cnc(-c2ccc3c(c2)CN(C2CCC(=O)NC2=O)C3=O)c1-c1cc2ccccc2[nH]1] | 440.3 |
| 528 | 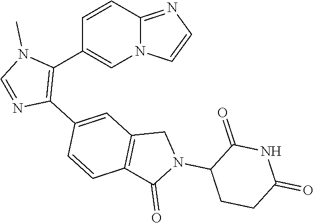 [SMILES: Cn1cnc(-c2ccc3c(c2)CN(C2CCC(=O)NC2=O)C3=O)c1-c1ccc2nccn2c1] | 441.2 |
| 529 | 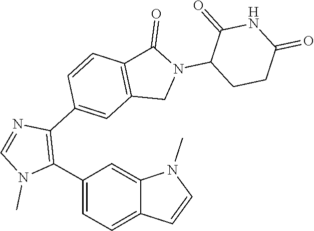 [SMILES: Cn1cnc(-c2ccc3c(c2)CN(C2CCC(=O)NC2=O)C3=O)c1-c1ccc2ccn(C)c2c1] | 454.3 |
| 530 | 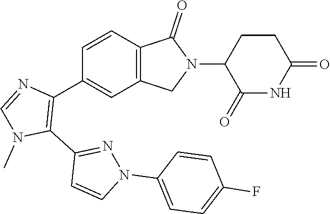 [SMILES: Cn1cnc(-c2ccc3c(c2)CN(C2CCC(=O)NC2=O)C3=O)c1-c1ccn(-c2ccc(F)cc2)n1] | 485.2 |
| 531 | 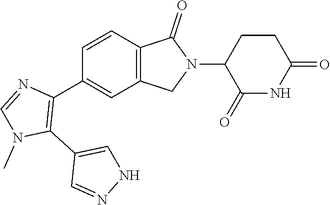 [SMILES: Cn1cnc(-c2ccc3c(c2)CN(C2CCC(=O)NC2=O)C3=O)c1-c1cn[nH]c1] | 391.2 |

### Example 532

### Synthesis of 3-(5-(1-methyl-2-(tetrahydro-2H-pyran-4-yl)-1H-imidazol-4-yl)-1-oxoisoindolin-2-yl)piperidine-2,6-dione

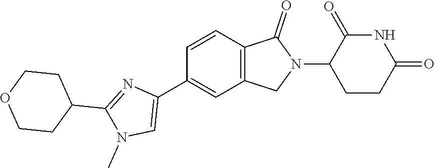 [SMILES: Cn1cc(-c2ccc3c(c2)CN(C2CCC(=O)NC2=O)C3=O)nc1C1CCOCC1]

A. 4,5-Dibromo-1-methyl-2-(tetrahydro-2H-pyran-4-yl)-1H-imidazole: A solution of 1-methyl-2-(tetrahydro-2H-pyran-4-yl)-1H-imidazole (1.00 g, 6.02 mmol, 1.00 eq) and NBS (2.25 g, 12.6 mmol, 2.00 eq) in DMF (10.0 mL) was stirred at 25° C. for 12 h. The reaction mixture was poured into H₂O (5.00 mL), extracted with EtOAc (3×10.0 mL), dried over Na₂SO₄ and concentrated in vacuum to give residue. The residue was purified by column chromatography (SiO₂, Dichloromethane:Methanol=100:1 to 80:1; TLC, Dichloromethane:Methanol=20:1, R_{f}=0.50) to give the title compound (0.90 g, 2.68 mmol, 44.6% yield) as yellow oil. (ESI⁺) m/z: 324.0 (M+H)⁺, (C₉H₁₂N₂Br₂).

B. 4-Bromo-1-methyl-2-(tetrahydro-2H-pyran-4-yl)-1H-imidazole: To a solution of 4,5-dibromo-1-methyl-2-(tetrahydro-2H-pyran-4-yl)-1H-imidazole (0.90 g, 2.78 mmol, 1.00 eq) in THF (10.0 mL) was added EtMgBr (740 mg, 5.56 mmol, 2.00 eq) dropwise at 0° C. under N₂. The reaction mixture was stirred at 0° C. for 2 h under N₂. The reaction mixture was quenched with NaHCO₃ (10.0 mL) and H₂O (10.0 mL). The aqueous phase was extracted with EtOAc (3×5.00 mL), dried over Na₂SO₄ and concentrated in vacuum to give a residue. The residue was purified by preparative-TLC (SiO₂, Dichloromethane:Methanol=20:1; TLC, Dichloromethane:Methanol=20:1, R_{f}=0.45) to give the title compound (150 mg, 611 μmol, 22.0% yield) as yellow oil. ¹H NMR: (400 MHz, DMSO-d₆) δ 6.76 (s, 1H), 4.09-4.06 (m, 2H), 3.60 (s, 3H), 3.54-3.58 (m, 2H), 2.90-2.84 (m, 1H), 2.09-2.01 (m, 2H), 1.77-1.72 (m, 2H). (ESI⁺) m/z: 245.0 (M+H)⁺, (C₉H₁₃BrN₂O).

C. 3-(5-(1-Methyl-2-(tetrahydro-2H-pyran-4-yl)-1H-imidazol-4-yl)-1-oxoisoindolin-2-yl)piperidine-2,6-dione: To a solution of 4-bromo-1-methyl-2-(tetrahydro-2H-pyran-4-yl)-1H-imidazole (150 mg, 611 μmol, 1.00 eq), 3-[1-oxo-5-(4,4,5,5-tetramethyl-1,3,2-dioxaborolan-2-yl)isoindolin-2-yl]piperidine-2,6-dione (679 mg, 1.84 mmol, 3.00 eq) in dioxane (3.00 mL) and H₂O (0.15 mL) was added Ru-Phos-Pd-G₃ (51.1 mg, 61.6 μmol, 0.10 eq), K₃PO₄ (259 mg, 1.22 mmol, 2.00 eq) under N₂. The reaction mixture was stirred at 100° C. for 2 h under N₂. The reaction mixture was concentrated in vacuum to give a residue. The residue was purified by preparative-HPLC (using a Phenomenex luna C18 (150 mm×25 mm×5 μm) and gradient of 0-30% acetonitrile in water (TFA) over 10 min at a flow rate of 25 mL/min to give the title compound (49.4 mg, 116 μmol, 19.1% yield, 96.6% purity in HPLC at 220 nm) as white solid. ¹H NMR: (400 MHz, DMSO-d₆) δ 11.0 (s, 1H), 8.13 (s, 1H), 8.12 (s, 1H), 8.11-7.85 (m, 2H), 5.16-5.11 (m, 1H), 4.45-4.38 (m, 2H), 4.01-3.98 (m, 2H), 3.85 (s, 3H), 3.65-3.63 (m, 2H), 2.94-2.89 (m, 1H), 2.59-2.50 (m, 1H), 2.45-2.42 (m, 1H), 2.01-2.84 (m, 6H). (ESI⁺) m/z: 409.1 (M+H)⁺, (C₂₂H₂₄N₄O₄).

### Example 533

### Synthesis of (3-[5-(1-methyl-5-pyrimidin-5-yl-pyrazol-3-yl)-1-oxo-isoindolin-2-yl]piperidine-2,6-dione

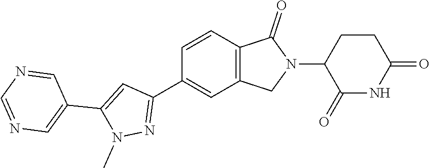 [SMILES: Cn1nc(-c2ccc3c(c2)CN(C2CCC(=O)NC2=O)C3=O)cc1-c1cncnc1]

A. 5-(5-Bromo-2-methyl-pyrazol-3-yl)pyrimidine: A solution of 3,5-dibromo-1-methyl-pyrazole (600 mg, 2.50 mmol, 1.00 eq), and pyrimidin-5-ylboronic acid (340 mg, 2.75 mmol, 1.10 eq), K₂CO₃ (1.04 g, 7.50 mmol, 3.00 eq) in dioxane (5.00 mL), H₂O (0.50 mL). degas and purge with N₂ 3 times, heat to 100° C. Then the reaction mixture was stirred at 100° C. for 0.25 h, degas and purge with N₂ 3 times. was added Pd(dppf)Cl₂·CH₂Cl₂ (204 mg, 250 μmol, 0.10 eq) at 100° C. Then the reaction mixture was stirred at 100° C. for 3 h. Cooled the reaction to 20° C., filtered and washed filter cake with ethyl acetate (20.0 mL). Diluted filtrate with H₂O (20.0 mL) and extracted with ethyl acetate (3×10.0 mL). The organic layer was washed with brine (3×10.0 mL) and dried the organic layer over Na₂SO₄ filtered and concentrated filtrate under vacuum at 45° C. to get residue. The residue was purified by prep-TLC (SiO₂, Petroleum ether:Ethyl acetate=2:1, R_{f}=0.30) to give title compound (5-(5-bromo-2-methyl-pyrazol-3-yl)pyrimidine (170 mg, 657 μmol, 26.3% yield, 92.5% purity in LCMS at 220 nm)¹H NMR: (400 MHz, DMSO-d₆) δ 9.28 (s, 1H), 9.05 (s, 2H), 6.83 (s, 1H), 3.90 (s, 3H), (ESI⁺) m/z: 241.0 (M+H)⁺, (C₈H₇BrN₄).

B. (3-[5-(1-Methyl-5-pyrimidin-5-yl-pyrazol-3-yl)-1-oxo-isoindolin-2-yl]piperidine-2,6-dione: A solution of 3-[5-[(4S)-4-hydroxy-2,2-dimethyl-4-piperidyl]-1-oxo-isoindolin-2-yl]piperidine-2,6-dione (130 mg, 330.05 μmol, 1.00 eq), DIEA (213 mg, 1.65 mmol, 287 μL, 5.00 eq), 6-acetyl-2-[[5-[4-[[4-(chloromethyl)phenyl]methyl]piperazin-1-yl]-2-pyridyl]amino]-8-cyclopentyl-5-methyl-pyrido[2,3-d]pyrimidin-7-one (263 mg, 396 μmol, 1.20 eq) in dioxane (5.00 mL) and H₂O (0.50 mL), degas and purge with N₂ 3 times. Then the reaction mixture was stirred at 80° C. for 2 h. Cooled the reaction to 20° C., filtered and washed filter cake with ethyl acetate (200 mL). Filter to collect filtrate and concentrate filtrate under vacuum at 45° C. to get residue. The crude product was purified by preparative-HPLC (using a Phenomenex luna C18 (150 mm×25 mm×5 μm and gradient of 15%-35% acetonitrile in water (TFA) over 10 min at a flow rate of 25.0 mL/min) to give title compound (3-[5-(1-methyl-5-pyrimidin-5-yl-pyrazol-3-yl)-1-oxo-isoindolin-2-yl]piperidine-2,6-dione (11.9 mg, 29.0 μmol, 4.08% yield, 97.5% purity in HPLC at 220 nm) as a white solid. ¹H NMR: (400 MHz, DMSO-d₆) δ 11.0 (s, 1H), 9.30 (s, 1H), 9.13 (s, 2H), 80.5 (s, 1H), 7.99 (s, 1H), 7.67 (s, 1H), 7.28 (s, 1H), 5.16-5.11 (m, 1H), 4.53-4.39 (m, 1H), 4.36-4.35 (m, 1H), 4.01 (s, 3H), 2.58-2.50 (m, 4H). (ESI⁺) m/z: 403.1 (M+H)⁺, (C₂₁H₁₈N₆O₃).

### Example 534

### Synthesis of 3-(5-(1-methyl-2-(spiro[2.5]octan-6-yl)-1H-imidazol-4-yl)-1-oxoisoindolin-2-yl)piperidine-2,6-dione

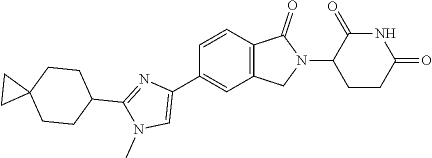 [SMILES: Cn1cc(-c2ccc3c(c2)CN(C2CCC(=O)NC2=O)C3=O)nc1C1CCC2(CC1)CC2]

A. tert-Butyl 5-amino-4-(5-(1-ethoxyvinyl)-1-oxoisoindolin-2-yl)-5-oxopentanoate: To a solution of tert-butyl 5-amino-4-(5-bromo-1-oxoisoindolin-2-yl)-5-oxopentanoate (4.00 g, 10.0 mmol, 1.00 eq) and tributyl(1-ethoxyvinyl)stannane (11.1 g, 30.7 mmol, 10.4 mL, 3.05 eq) in dioxane (80.0 mL) was added Pd(PPh₃)₄ (930 mg, 805 μmol, 0.08 eq) under N₂. The mixture was stirred at 90° C. for 12 h. The reaction mixture was added to saturated NH₄Cl solution (160 mL). The reaction mixture was partitioned between Ethyl acetate (200 mL) and H₂O (200 mL), the water phase was extracted with Ethyl acetate (3×200 mL). The combined organic layers were dried over Na₂SO₄, filter and concentrated. The residue was purified by column chromatography (SiO₂, Dichloromethane:Methanol=15:1, TLC:Dichloromethane:Methanol=15:1, R_{f}=0.50) to give the title compound (3.70 g, 5.16 mmol, 51.2% yield, 70.0% purity in LCMS at 220 nm) as yellow solid. ¹H NMR: (400 MHz, DMSO-d₆) δ 7.83-7.82 (m, 1H), 7.74-7.72 (m, 1H), 7.68-7.62 (m, 1H), 7.19-7.17 (m, 2H), 4.88-4.88 (m, 1H), 4.74-4.72 (m, 1H), 4.62-4.57 (m, 1H), 4.49-4.44 (m, 1H), 4.41-4.40 (m, 1H), 3.95-3.90 (m, 2H), 2.17-2.12 (m, 3H), 2.00-1.96 (m, 1H), 1.38-1.32 (m, 12H). (ESI⁺) m/z: 389.1 (M+H)⁺, (C₂₁H₂₈N₂O₅).

B. tert-butyl 5-amino-4-(5-(2-bromoacetyl)-1-oxoisoindolin-2-yl)-5-oxopentanoate: To a solution of tert-butyl 5-amino-4-(5-(1-ethoxyvinyl)-1-oxoisoindolin-2-yl)-5-oxopentanoate (1.00 g, 2.57 mmol, 1.00 eq) in THF (20.0 mL) and H₂O (4.00 mL) was added NBS (366 mg, 2.06 mmol, 0.8 eq) at 0° C. The mixture was stirred at 25° C. for 2 h. The reaction mixture was poured into H₂O (30.0 mL) and extracted with Ethyl acetate (3×50.0 mL). The combined organic layer was washed with brine (3×50.0 mL), dried over Na₂SO₄, filtered and concentrated to give the title compound (450 mg, 683 μmol, 26.6% yield, 60.9% purity in LCMS at 220 nm) as off-white solid. (ESI⁺) m/z: 440.3 (M+H)⁺, (C₁₉H₂₃BrN₂O₅).

C. tert-Butyl 5-amino-4-(5-(methylglycyl)-1-oxoisoindolin-2-yl)-5-oxopentanoate: To a solution of MeNH₂/THF (2 M, 32.2 mL, 70.7 eq) in DMF (2.00 mL) was added tert-butyl 5-amino-4-(5-(2-bromoacetyl)-1-oxoisoindolin-2-yl)-5-oxopentanoate (400 mg, 910 μmol, 1.00 eq) in DMF (3.00 mL) at −10° C. The mixture was stirred at −10° C. for 0.5 h. The mixture was added HCl/dioxane (1.00 mL). The mixture was poured into ice cooled H₂O (20.0 mL), and extracted with dichloromethane (3×25.0 mL). The combined organic layer was washed with saturated NaHCO₃ solution (25.0 mL) and NaCl (25.0 mL), dried over Na₂SO₄ and concentrated under reduced pressure to give the title compound (300 mg, 770 μmol, 84.6% yield, 80.0% purity in LCMS at 220 nm) as light yellow oil. (ESI⁺) m/z: 390.4 (M+H)⁺, (C₂₀H₂₇N₃₀₅).

D. tert-Butyl 5-amino-4-(5-(N-methyl-N-(spiro[2.5]octane-6-carbonyl)glycyl)-1-oxoisoindolin-2-yl)-5-oxopentanoate: To a solution of spiro[2.5]octane-6-carboxylic acid (356 mg, 2.31 mmol, 3.00 eq), CMPI (984 mg, 3.85 mmol, 5.0 eq) and DIEA (497 mg, 3.85 mmol, 670 μL, 5.00 eq) in DMF (3.00 mL) at 0.5 h. Then the mixture was added tert-butyl 5-amino-4-(5-(methylglycyl)-1-oxoisoindolin-2-yl)-5-oxopentanoate (300 mg, 770. μmol, 1.00 eq). Then the mixture was stirred at 25° C. for 1 h. The mixture was poured into H₂O (10.0 mL), and extracted with Ethyl acetate (3×30.0 mL). The combined organic layer was washed with saturated NaCl (3×30.0 mL), dried over Na₂SO₄ and concentrated under reduced pressure to get residue. The residue was purified by preparative-TLC (Dichloromethane:Methanol=10:1,R_{f}=0.50) to give the title compound (150 mg, 224 μmol, 29.0% yield, 78.5% purity in LCMS at 220 nm) as light yellow oil. (ESI⁺) m/z: 526.6 (M+H)⁺, (C₂₉H₃₉N₃O₆).

E. tert-Butyl 5-amino-4-(5-(1-methyl-2-(spiro[2.5]octan-6-yl)-1H-imidazol-4-yl)-1-oxoisoindolin-2-yl)-5-oxopentanoate: To a solution of tert-butyl 5-amino-4-(5-(N-methyl-N-(spiro[2.5]octane-6-carbonyl)glycyl)-1-oxoisoindolin-2-yl)-5-oxopentanoate (150 mg, 285 μmol 1.00 eq) and NH₄OAc (153 mg, 2.00 mmol, 7.00 eq) in HCONH₂ (15.0 mL) under N₂. The mixture was stirred at 100° C. for 40 h under N₂. The mixture was poured into H₂O (20.0 mL), and extracted with Ethyl acetate (3×30.0 mL). The combined organic layer was dried over Na₂SO₄ and concentrated under reduced pressure to get residue. The residue was purified by preparative-TLC (Dichloromethane:Methanol=15:1,R_{f}=0.40) to give the title compound (60.0 mg, 94.2 μmol, 33.1% yield, 79.9% purity in LCMS at 220 nm) as light yellow oil. (ESI⁺) m/z: 507.2 (M+H)⁺, (C₂₉H₃₈N₄O₄).

F. 3-(5-(1-Methyl-2-(spiro[2.5]octan-6-yl)-1H-imidazol-4-yl)-1-oxoisoindolin-2-yl)piperidine-2,6-dione: To a solution of tert-butyl 5-amino-4-(5-(1-methyl-2-(spiro[2.5]octan-6-yl)-1H-imidazol-4-yl)-1-oxoisoindolin-2-yl)-5-oxopentanoate (60.0 mg, 118 μmol, 1.00 eq) in ACN (3.00 mL) was added TsOH (61.1 mg, 355 μmol, 3.00 eq) under N₂. The mixture was stirred at 80° C. for 2 h under N₂. The reaction mixture was concentrated under reduced pressure to get a residue at 45° C. The residue was purified by preparative-HPLC (using a Phenomenex luna C18 (150×25 mm×10 μm) and gradient of 18.0%-38.0% acetonitrile in water containing 0.50% TFA over 2 min at a flow rate of 25 mL/min) to give the title compound (17.9 mg, 39.5 μmol, 33.3% yield, 95.4% purity in HPLC at 220 nm) as white solid. ¹H NMR: (400 MHz, DMSO-d₆) δ 11.01 (s, 1H), 8.20-8.08 (m, 1H), 8.03-8.01 (m, 1H), 7.92-7.85 (m, 2H), 5.16-5.11 (m, 1H), 4.55-4.38 (m, 2H), 3.87-3.78 (m, 3H), 3.23-3.14 (m, 1H), 2.94-2.89 (m, 1H), 2.63-2.59 (m, 1H), 2.45-2.35 (m, 1H), 2.10-2.04 (m, 1H), 1.95-1.82 (m, 6H), 1.01-0.99 (m, 2H), 0.38-0.26 (m, 4H). (ESI⁺) m/z: 433.2 (M+H)⁺, (C₂₅H₂₈N₄O₃).

### Example 535

### Synthesis of 3-(5-(1-methyl-5-(pyrimidin-5-yl)-2-(tetrahydro-2H-pyran-4-yl)-1H-imidazol-4-yl)-1-oxoisoindolin-2-yl)piperidine-2,6-dione

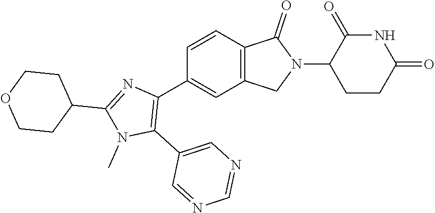 [SMILES: Cn1c(C2CCOCC2)nc(-c2ccc3c(c2)CN(C2CCC(=O)NC2=O)C3=O)c1-c1cncnc1]

A. 5-(1-Methyl-2-(tetrahydro-2H-pyran-4-yl)-1H-imidazol-5-yl)pyrimidine (Five reactions were carried out in parallel): To a solution of 5-bromo-1-methyl-2-(tetrahydro-2H-pyran-4-yl)-1H-imidazole (100 mg, 407 μmol, 1.00 eq) and pyrimidin-5-ylboronic acid (202 mg, 1.63 mmol, 4.00 eq) in dioxane (2.00 mL) and H₂O (0.20 mL) was added K₃PO₄ (173 mg, 815 μmol, 2.00 eq) and Pd(PPh₃)₄ (47.1 mg, 40.8 μmol, 0.10 eq) under N₂. The reaction mixture was stirred at 90° C. for 12 h under N₂. The residue was purified by preparative-TLC (SiO₂, Dichloromethane:Methanol=20:1; TLC, Dichloromethane:Methanol=20:1, R_{f}=0.30) to give the title compound (100 mg, 315 μmol, 15.4% yield). (ESI⁺) m/z: 245.0 (M+H)⁺, (C₁₃H₁₆N₄O).

B. 5-(4-Bromo-1-methyl-2-(tetrahydro-2H-pyran-4-yl)-1H-imidazol-5-yl)pyrimidine: To a solution of 5-(1-methyl-2-(tetrahydro-2H-pyran-4-yl)-1H-imidazol-5-yl)pyrimidine (100 mg, 409 μmol, 1.00 eq) in DMF (2.00 mL) was added a solution of NBS (58.2 mg, 327 μmol, 0.80 eq) in DMF (2.00 mL) at 0° C. The reaction mixture was stirred at 25° C. for 2 h. The residue was purified by preparative-TLC (SiO₂, Dichloromethane:Methanol=20:1; TLC, Dichloromethane:Methanol=20:1, R_{f}=0.40) to give the title compound (60.0 mg, 185 μmol, 45.3% yield). ¹H NMR: (400 MHz, CDCl₃) (9.24 (s, 1H), 8.94 (s, 2H), 3.94-3.91 (m, 2H), 3.57 (s, 3H), 3.50-3.45 (m, 2H), 3.32-3.13 (m, 1H), 1.77-1.73 (m, 4H). (ESI⁺) m/z: 324.8 (M+H)⁺, (C₁₃H₁₅BrN₄O).

C. 3-(5-(1-Methyl-5-(pyrimidin-5-yl)-2-(tetrahydro-2H-pyran-4-yl)-1H-imidazol-4-yl)-1-oxoisoindolin-2-yl)piperidine-2,6-dione: To a solution of 5-(4-bromo-1-methyl-2-(tetrahydro-2H-pyran-4-yl)-1H-imidazol-5-yl)pyrimidine (50.0 mg, 154 μmol, 1.00 eq), 3-[1-oxo-5-(4,4,5,5-tetramethyl-1,3,2-dioxaborolan-2-yl)isoindolin-2-yl]piperidine-2,6-dione (114 mg, 309 μmol, 2.00 eq) in dioxane (1.00 mL) and H₂O (0.05 mL) was added Ru-Phos-Pd-G₃ (12.9 mg, 15.4 μmol, 0.10 eq), K₃PO₄ (65.6 mg, 309 μmol, 2.00 eq) under N₂. The reaction mixture was stirred at 100° C. for 2 h under N₂. The reaction mixture was concentrated in vacuum to give residue. The residue was purified by preparative-HPLC (using a Welch Xtimate C18 (150 mm×25 mm×5 μm) and gradient of 5-25% acetonitrile in water containing 0.05% TFA over 10 min at a flow rate of 25.0 mL/min) to give the title compound (17.9 mg, 34.9 μmol, 32.6% yield, 95.0% purity in HPLC at 220 nm) as white solid. ¹H NMR: (400 MHz, MeOD) δ 9.31 (s, 1H), 8.87 (s, 2H), 7.84 (d, J=8.0 Hz, 1H), 7.61 (s, 1H), 7.53 (d, J=7.6 Hz, 1H), 5.18-5.13 (m, 1H), 4.53-4.47 (m, 2H), 4.14-4.11 (m, 2H), 3.78 (s, 3H), 3.69-3.60 (m, 3H), 2.90-2.80 (m, 1H), 2.79-2.78 (m, 1H), 2.50-2.35 (m, 1H), 2.18-2.04 (m, 6H). (ESI⁺) m/z: 487.2 (M+H)⁺, (C₂₆H₂₆N₆O₄).

### Example 536

### Synthesis of 3-(5-(1-(difluoromethyl)-3-(pyrimidin-5-yl)-1H-pyrazol-4-yl)-1-oxoisoindolin-2-yl)piperidine-2,6-dione

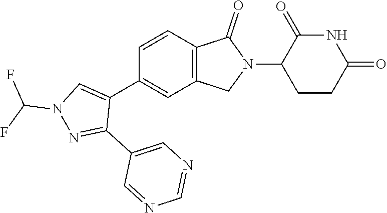 [SMILES: O=C1CCC(N2Cc3cc(-c4cn(C(F)F)nc4-c4cncnc4)ccc3C2=O)C(=O)N1]

A. (E)-3-(Dimethylamino)-1-(pyrimidin-5-yl)prop-2-en-1-one: To a solution of 1-(pyrimidin-5-yl)ethan-1-one (2.50 g, 20.4 mmol, 1.00 eq) in i-PrOH (100 mL) was added DMF-DMA (3.27 g, 27.4 mmol, 3.64 mL, 1.34 eq). The mixture was stirred at 100° C. for 24 h. The mixture was filtered to collect liquid and concentrated under reduced pressure to get residue. The residue was dissolved in Ethyl acetate (5.00 mL) and methyl ter-butyl ether (10.0 mL) was added dropwise to get white slurry, after filtering to get the title compound (3.50 g, 19.47 mmol, 47.5% yield, 98.6% purity at 220 nm in LCMS) as brown solid. ¹H NMR: (400 MHz, DMSO-d₆) δ 9.27 (s, 1H), 9.21 (s, 2H), 7.83-7.80 (d, J=12.0 Hz, 1H), 5.91-5.88 (d, J=12.0 Hz, 1H), 3.18 (s, 3H), 2.96 (s, 3H). (ESI⁺) m/z: 178.1 (M+H)⁺, (C₉H₁IN₃₀).

B. 5-(1H-Pyrazol-5-yl) pyrimidine: To a solution of (E)-3-(dimethylamino)-1-(pyrimidin-5-yl)prop-2-en-1-one (3.50 g, 19.7 mmol, 1.00 eq) in EtOH (70.0 mL) was added N₂H₄·H₂O (3.03 g, 59.2 mmol, 2.93 mL, 98.0% purity, 3.00 eq) dropwise. The mixture was stirred at 25° C. for 2 h. The reaction mixture was poured ice cooled H₂O (100 mL) and extracted with Ethyl acetate (2×50.0 mL). The combined organic layer was washed with saturated NaCl aqueous (2×40.0 mL), dried over Na₂SO₄ and concentrated under reduced pressure to get the title compound (2.85 g, 19.3 mmol, 97.8% yield, 99.1% purity at 220 nm in LCMS) was obtained as brown solid (ESI⁺) m/z: 147.3 (M+H)⁺, (C₇H₆N₄).

C. 5-(4-Bromo-1H-pyrazol-5-yl)pyrimidine: To a solution of 5-(4-bromo-1H-pyrazol-5-yl)pyrimidine (700 mg, 4.79 mmol, 1.00 eq) in DMF (7.00 mL) was added NBS (767 mg, 4.31 mmol, 0.90 eq) at 25° C. The mixture was stirred at 25° C. for 2 h. The mixture was poured into H₂O (80.0 mL) and extracted with Ethyl acetate (2×50.0 mL). The combined organic layer was washed with saturated NaCl aqueous (2×50.0 mL), dried over Na₂SO₄ and concentrated under reduced pressure to get residue. The crude product was purified by column chromatography (SiO₂, Dichloromethane:Methanol=10:1, R_{f}=0.40) to give the title compound (2.85 g, 19.3 mmol, 92.7% yield, 99.1% purity at 220 nm in LCMS) as brown solid. ¹H NMR: (400 MHz, DMSO-d₆) δ 11.1 (s, 1H), 9.26-9.16 (m, 3H), 8.15 (s, 1H). (ESI⁺) m/z: 227.0 (M+H)⁺, (C₇H₅BrN₄).

D. 5-(4-Bromo-1-(difluoromethyl)-1H-pyrazol-3-yl)pyrimidine: To a solution of 5-(4-bromo-1H-pyrazol-5-yl)pyrimidine (500 mg, 2.22 mmol, 1.00 eq) in ACN (20.0 mL) was added K₂CO₃ (614 mg, 4.44 mmol, 2.00 eq) and sodium; 2-chloro-2,2-difluoro-acetate (677.46 mg, 4.44 mmol, 2.00 eq), 18-Crown-6 (33.7 mg, 127 μmol, 2.00 eq). The mixture was stirred at 80° C. for 12 h. The mixture was filtered to collect liquid and concentrated under reduced pressure to get residue. The residue was purified by chromatography column (SiO₂, Petroleum ether:Ethyl acetate=1:1, R_{f}=0.20) to give the title compound (300 mg, 1.09 mmol, 24.5% yield, 100% purity at 220 nm in LCMS) as yellow solid. ¹H NMR: (400 MHz, DMSO-d₆) δ 9.30 (s, 1H), 9.21 (s, 1H), 8.81 (s, 1H), 8.06-7.77 (m, 1H), 8.15 (s, 1H). (ESI⁺) m/z: 276.9 (M+H)⁺, (C₈H₅BrF₂N₄).

E. 3-(5-(1-(Difluoromethyl)-3-(pyrimidin-5-yl)-1H-pyrazol-4-yl)-1-oxoisoindolin-2-yl)piperidine-2,6-dione: To a solution of 5-(4-bromo-1-(difluoromethyl)-1H-pyrazol-3-yl)pyrimidine (150 mg, 545 μmol, 1.00 eq), 3-(1-oxo-5-(4,4,5,5-tetramethyl-1,3,2-dioxaborolan-2-yl)isoindolin-2-yl)piperidine-2,6-dione (706 mg, 1.91 mmol, 3.50 eq) and K₃PO₄ (347 mg, 1.64 mmol, 3.00 eq) in dioxane (5.00 mL) and H₂O (0.25 mL) was added Ru-Phos-Pd-G₃ (45.6 mg, 54.5 μmol, 0.10 eq) under N₂. The mixture was stirred at 100° C. for 2 h under N₂. The mixture was filtered to collect liquid and concentrated under reduced pressure to get residue. The crude product was purified by preparative-HPLC (using a Phenomenex luna C18 (150 mm×25 mm 10 μm) and gradient of 16-46% acetonitrile in water containing 0.5% TFA over 15 min at a flow rate of 25 mL/min) to give the title compound (57.9 mg, 131 μmol, 11.5% yield, 99.8% purity in HPLC at 220 nm) as white solid. ¹H NMR: (400 MHz, DMSO-d₆) δ 11.0 (s, 1H), 9.23 (s, 1H), 8.82 (s, 2H), 8.75 (s, 1H), 8.13-8.01 (m, 1H), 7.99-7.84 (m, 1H), 7.75-7.73 (m, 1H), 7.45-7.43 (m, 1H), 5.15-5.10 (m, 1H), 4.46-4.29 (m, 2H), 2.95-2.88 (m, 1H), 2.61-2.57 (m, 1H), 2.42-2.37 (m, 1H), 2.03-2.01 (m, 1H). (ESI⁺) m/z: 438.9 (M+H)⁺, (C₂₁H₁₆F₂N₆O₃).

### Example 537

### Synthesis of 3-(5-(1-methyl-2-(piperidin-1-yl)-1H-imidazol-5-yl)-1-oxoisoindolin-2-yl)piperidine-2,6-dione

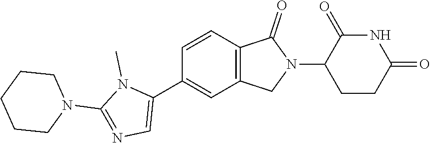 [SMILES: Cn1c(-c2ccc3c(c2)CN(C2CCC(=O)NC2=O)C3=O)cnc1N1CCCCC1]

A. N-(2,2-Dimethoxyethyl)-N-methylpiperidine-1-carboximidamide: To a mixture of piperidine-1-carbonitrile (500 mg, 4.54 mmol, 1.00 eq) and 2,2-dimethoxy-N-methyl-ethanamine (540 mg, 4.54 mmol, 583 μL, 1.00 eq) was added a mixture of 2,2-dimethoxy-N-methyl-ethanamine (540 mg, 4.54 mmol, 583 μL, 1.00 eq) and HCl (12 M, 378 μL, 1.00 eq). The mixture was stirred at 100° C. for 12 h under N₂. After the reaction was completed, the reaction mixture was filtered and the filtrate was concentrated in vacuum to give the title compound (1.00 g, crude) as yellow oil. (ESI⁺) m/z: 230.1 (M+H)⁺, (C₁₁H₂₃N₃O₂).

B. 1-(1-methyl-1H-imidazol-2-yl)piperidine: To a solution of N-(2,2-dimethoxyethyl)-N-methyl-piperidine-1-carboxamidine (1.00 g, 4.36 mmol, 1.00 eq) in MeOH (10.0 mL) was added HCl (12 M, 1.82 mL, 5.00 eq) under N₂. The mixture was stirred at 80° C. for 1 h under N₂. After the reaction was completed, the reaction mixture was adjusted to pH=7 with saturated NaHCO₃ aqueous solution (20.0 mL) and extracted with ethyl acetate (3×20.0 mL). The organic layer was washed with brine (30.0 mL), dried over Na₂SO₄, filtered and the filtrate was concentrated in vacuum to give the title compound (400 mg, 2.41 mmol, 55.2% yield, 99.6% purity in LCMS at 220 nm) as yellow oil. ¹H NMR: (400 MHz, CDCl₃) δ 6.75 (s, 1H), 6.63 (s, 1H), 3.46 (s, 3H), 2.98 (t, J=5.6 Hz, 4H), 1.71-1.64 (m, 4H), 1.59-1.54 (m, 2H). (ESI⁺) m/z: 166.1 (M+H)⁺, (C₉H₁₅N₃).

C. tert-Butyl 5-amino-4-(5-(1-methyl-2-(piperidin-1-yl)-1H-imidazol-5-yl)-1-oxoisoindolin-2-yl)-5-oxopentanoate: To a solution of 1-(1-methylimidazol-2-yl)piperidine (100 mg, 594 μmol, 1.00 eq) and tert-butyl 5-amino-4-(5-bromo-1-oxo-isoindolin-2-yl)-5-oxo-pentanoate (236 mg, 594 μmol, 1.00 eq) in NMP (1.00 mL) was added Pd(OAc)₂ (1.34 mg, 5.95 μmol, 0.01 eq), Davephos (4.68 mg, 11.9 μmol, 0.02 eq), Bu₄NOAc (358 mg, 1.19 mmol, 362 μL, 2.00 eq) and isobutric acid (15.7 mg, 178 μmol, 16.5 μL, 0.30 eq) under N₂. The mixture was stirred at 100° C. for 16 h under N₂. After the reaction was completed, the reaction mixture was poured into saturated NaCl solution (5.00 mL). The aqueous layer was extracted with ethyl acetate (3×10.0 mL) and the combined organic layer was washed with brine (3×10.0 mL), dried over Na₂SO₄, filtered and the filtrate was concentrated in vacuum to get a residue. The residue was purified by preparative-TLC (Dichloromethane:Methanol=10:1, R_{f}=0.50) to give the title compound (90.0 mg, 185 μmol, 31.1% yield, 99.3% purity in LCMS at 220 nm) as yellow oil. ¹H NMR: (400 MHz, DMSO-d₆) δ 7.71 (d, J=8.0 Hz, 1H), 7.67 (s, 1H), 7.57 (d, J=8.4 Hz, 2H), 7.20 (s, 1H), 6.92 (s, 1H), 4.73 (dd, J=10.4, 4.0 Hz, 1H), 4.55 (dd, J=58.0, 17.6 Hz, 2H), 3.48 (s, 3H), 3.02 (t, J=5.2 Hz, 4H), 2.20-2.14 (m, 2H), 1.66 (s, 4H), 1.56 (d, J=4.0 Hz, 2H), 1.32 (s, 9H), 1.17 (t, J=7.2 Hz, 2H). (ESI⁺) m/z: 482.2 (M+H)⁺, (C₂₆H₃₅N₅O₄).

D. 3-(5-(1-Methyl-2-(piperidin-1-yl)-1H-imidazol-5-yl)-1-oxoisoindolin-2-yl)piperidine-2,6-dione: To a solution of tert-butyl 5-amino-4-[5-[3-methyl-2-(1-piperidyl)imidazol-4-yl]-1-oxo-isoindolin-2-yl]-5-oxo-pentanoate (90.0 mg, 185 μmol, 1.00 eq) in ACN (1.00 mL) was added TsOH (319 mg, 1.86 mmol, 10.0 eq). The mixture was stirred at 80° C. for 2 h under N₂. After the reaction was completed, the reaction mixture was filtered and the filtrate was concentrated in vacuum to get a residue. The residue was purified by preparative-TLC (SiO₂, Dichloromethane:Methanol=10:1, R_{f}=0.50) and by preparative-HPLC (using a Welch Xtimate C18 (150 mm×25 mm×5 μm) and gradient of 3-33% acetonitrile in water containing 0.5% TFA over 2 min at a flow rate of 25.0 mL/min) to give the title compound (42.9 mg, 101 μmol, 54.7% yield, 96.3% purity in HPLC at 220 nm) as a white solid. ¹H NMR: (400 MHz, DMSO-d₆) δ 11.0 (s, 1H), 7.88 (d, J=8.0 Hz, 1H), 7.80 (s, 1H), 7.70 (d, J=8.0 Hz, 1H), 7.66 (d, J=6.0 Hz, 1H), 5.15 (dd, J=12.8, 4.8 Hz, 1H), 4.47 (dd, J=33.2, 17.2 Hz, 2H), 3.55 (s, 3H), 3.33-3.30 (m, 4H), 2.95-2.90 (m, 1H), 2.63 (s, 1H), 2.47-2.43 (m, 1H), 2.04 (s, 1H), 1.71-1.62 (m, 6H). (ESI⁺) m/z: 408.2 (M+H)⁺, (C₂₂H₂₅N₅O₃).

### Example 538

### Synthesis of 3-(5-(1-methyl-5-(tetrahydro-2H-pyran-4-yl)-1H-pyrazol-3-yl)-1-oxoisoindolin-2-yl)piperidine-2,6-dione

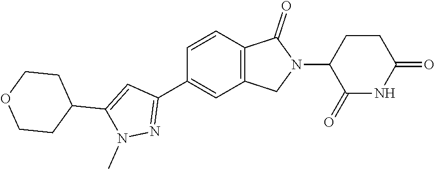 [SMILES: Cn1nc(-c2ccc3c(c2)CN(C2CCC(=O)NC2=O)C3=O)cc1C1CCOCC1]

A. 3-Oxo-3-(tetrahydro-2H-pyran-4-yl)propanenitrile: To a cooled solution of ethyl tetrahydro-2H-pyran-4-carboxylate (6.00 g, 37.9 mmol, 1.00 eq) in THF (90.0 mL) was added MeCN (3.11 g, 75.8 mmol, 3.99 mL, 2.00 eq). After 30 min, the reaction was added LHMDS (1 M, 75.8 mL, 2.00 eq). Then the reaction mixture was stirred at 0° C. for 3 h under N₂. The mixture was quenched with saturated citric acid solution until pH=5.00 and extracted with Ethyl acetate (3×200 mL). The combined organic layer was dried over Na₂SO₄, filtered and concentrated. The crude product was purified by column chromatography (SiO₂, Petroleum ether:Ethyl acetate=100:1 to 1:1, TLC:Petroleum ether: Ethyl acetate=1:1, R_{f}=0.20) to give the title compound (2.90 g, 18.9 mmol, 49.9% yield) as light yellow oil. ¹H NMR: (400 MHz, DMSO-d₆) δ 4.14-4.12 (m, 2H), 3.86-3.82 (m, 2H), 3.30-3.26 (m, 2H), 2.70-2.65 (m, 1H), 1.75-1.71 (m, 2H), 1.49-1.43 (m, 2H). (ESI⁺) m/z: 154.1 (M+H)⁺ (C₈H₁₁NO₂).

B. 5-(Tetrahydro-2H-pyran-4-yl)-1H-pyrazol-3-amine: To a solution of 3-oxo-3-(tetrahydro-2H-pyran-4-yl) propanenitrile (2.60 g, 16.9 mmol, 1.00 eq) in EtOH (25.0 mL) was added N₂H₄/H₂O (3.28 g, 64.2 mmol, 3.18 mL, 98.0% purity, 3.78 eq) under N₂. The mixture was stirred at 90° C. for 12 h under N₂. The mixture was poured into ice cooled H₂O (50.0 mL), and extracted with Ethyl acetate (2×50.0 mL). The combined organic layer was washed with saturated NaCl solution (2×50.0 mL), dried over Na₂SO₄ and concentrated under reduced pressure to give the title compound (1.70 g, 8.03 mmol, 47.3% yield, 79.0% purity in LCMS at 220 nm) as light yellow oil. ¹H NMR: (400 MHz, DMSO-d₆) δ 11.36-10.74 (m, 1H), 5.18 (m, 1H), 4.78-4.22 (m, 2H), 3.87-3.82 (m, 2H), 3.39-3.33 (m, 2H), 2.70-2.66 (m, 1H), 1.74-1.71 (m, 2H), 1.59-1.52 (m, 2H). (ESI⁺) m/z: 168.0 (M+H)⁺, (C₈H₁₃N₃₀).

C. 3-Iodo-5-(tetrahydro-2H-pyran-4-yl)-1H-pyrazole: To a solution of 5-(tetrahydro-2H-pyran-4-yl)-1H-pyrazol-3-amine (400 mg, 2.39 mmol, 1. eq) and TsOH (1.03 g, 5.98 mmol, 2.50 eq) in ACN (20.0 mL) and dropwise a solution of NaNO₂ (412 mg, 5.98 mmol, 2.5 eq) in H₂O (4.00 mL) slowly at 0° C. The mixture was stirred at 0° C. for 0.5 h. Then dropwise solution of NaI (1.79 g, 11.9 mmol, 5.00 eq) in H₂O (4.00 mL) at 0° C. Then the mixture was stirred at 25° C. for 3 h. The mixture was poured into ice cooled H₂O (50.0 mL), and extracted with Ethyl acetate (2×50.0 mL). The combined organic layer was washed with saturated NaCl solution (2×50.0 mL), dried over Na₂SO₄ and concentrated under reduced pressure to get residue. The crude product was purified by column chromatography (SiO₂, Dichloromethane:Methanol=500:1 to 20:1, TLC: Dichloromethane:Methanol=20:1,R_{f}=0.20) to give the title compound (210 mg, 634 μmol, 13.2% yield, 84.0% purity in LCMS at 220 nm) as red solid. (ESI⁺) m/z: 278.9 (M+H)+(C₈H₁₁IN₂O).

D. 3-Iodo-1-methyl-5-(tetrahydro-2H-pyran-4-yl)-1H-pyrazole: To a solution of 3-iodo-5-(tetrahydro-2H-pyran-4-yl)-1H-pyrazole (200 mg, 719.19 μmol, 1.00 eq) in DMF (3.00 mL) was added K₂CO₃ (109 mg, 791 μmol, 1.10 eq) was stirred at 25° C. for 30 min under N₂. Then the reaction mixture was added CH₃I (510 mg, 3.60 mmol, 223 μL, 5.00 eq) at 25° C. under N₂. The mixture was stirred at 25° C. for 12 h under N₂. The mixture was poured into H₂O (10.0 mL), and extracted with Ethyl acetate (3×20.0 mL). The combined organic layer was washed with saturated NaCl solution (3×20.0 mL), dried over Na₂SO₄ and concentrated under reduced pressure to get residue. The mixture was purified by preparative-TLC (Dichloromethane:Methanol=20:1, R_{f}=0.50) to give the title compound (100 mg, 263 μmol, 36.6% yield, 77.0% purity in LCMS at 220 nm) as light yellow oil. ¹H NMR: (400 MHz, DMSO-d₆) δ 6.28-6.25 (m, 1H), 3.92-3.87 (m, 4H), 3.78-3.76 (m, 3H), 3.01-2.92 (m, 1H), 1.74-1.60 (m, 2H), 1.57-1.51 (m, 2H). (ESI⁺) m/z: 293.0 (M+H)+(C₉H₁₃IN₂O).

E. 3-(5-(1-Methyl-5-(tetrahydro-2H-pyran-4-yl)-1H-pyrazol-3-yl)-1-oxoisoindolin-2-yl)piperidine-2,6-dione: To a solution of 3-iodo-1-methyl-5-(tetrahydro-2H-pyran-4-yl)-1H-pyrazole (40 mg, 136 μmol, 1.00 eq), 3-(1-oxo-5-(4,4,5,5-tetramethyl-1,3,2-dioxaborolan-2-yl)isoindolin-2-yl)piperidine-2,6-dione (202 mg, 547 μmol, 4.00 eq) and K₃PO₄ (87.2 mg, 410 μmol, 3.00 eq) in dioxane (2.00 mL) and H₂O (0.10 mL) was added Ru-Phos-Pd-G₃ (11.4 mg, 13.7 μmol, 0.10 eq) under N₂. The mixture was stirred at 100° C. for 4 h under N₂. The reaction mixture was concentrated under reduced pressure to get a residue at 45° C. The residue was purified by preparative-HPLC (using a Phenomenex luna C18 (150×25 mm×10 μm) and gradient of 12.0%-42.0% acetonitrile in water containing 0.50% TFA over 10 min at a flow rate of 25 mL/min) to give the title compound (8.59 mg, 20.4 μmol, 14.9% yield, 97.0% purity in HPLC at 220 nm) was obtained as white solid. ¹H NMR: (400 MHz, DMSO) δ 10.99 (s, 1H), 8.01-7.99 (m, 1H), 7.98-7.90 (m, 1H), 7.72-7.70 (m, 1H), 6.72-6.69 (m, 1H), 5.14-5.09 (m, 1H), 4.50-4.32 (m, 2H), 3.96-3.92 (m, 2H), 3.85 (s, 3H), 3.57-3.46 (m, 2H), 3.06-3.03 (m, 1H), 3.02-2.91 (m, 1H), 2.57-2.51 (m, 1H), 2.38-2.36 (m, 1H), 2.05-2.02 (m, 1H), 1.84-1.81 (m, 2H), 1.65-1.61 (m, 2H). (ESI⁺) m/z: 409.2 (M+H)⁺, (C₂₂H₂₄N₄O₄).

### Example 539

### Synthesis of 3-(5-(2-(4,4-dimethylcyclohexyl)-1-methyl-1H-imidazol-4-yl)-1-oxoisoindolin-2-yl)piperidine-2,6-dione

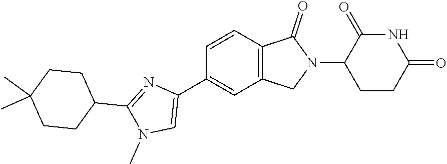 [SMILES: Cn1cc(-c2ccc3c(c2)CN(C2CCC(=O)NC2=O)C3=O)nc1C1CCC(C)(C)CC1]

A. tert-Butyl 5-amino-4-(5-(N-(4, 4-dimethylcyclohexane-1-carbonyl)-N-methylglycyl)-1-oxoisoindolin-2-yl)-5-oxopentanoate: To a solution of 4, 4-dimethylcyclohexane-1-carboxylic acid (401 mg, 2.57 mmol, 4.00 eq) and DIEA (332 mg, 2.57 mmol, 447 μL, 4 eq) in DMF (3.00 mL) was added CMPI (656 mg, 2.57 mmol, 4.00 eq). After 0.5 h tert-butyl 5-amino-4-(5-(methylglycyl)-1-oxoisoindolin-2-yl)-5-oxopentanoate (250 mg, 642 μmol, 1.00 eq) was added into the mixture. Then the mixture was stirred at 25° C. for 1 h. The mixture was poured into H₂O (10.0 mL), and extracted with Ethyl acetate (3×30.0 mL). The combined organic layer was washed with saturated NaCl (3×30.0 mL), dried over Na₂SO₄ and concentrated under reduced pressure to get residue. The residue was purified by preparative-TLC (Dichloromethane:Methanol=10:1,R_{f}=0.50) to give the title compound (300 mg, 379 μmol, 29.5% yield, 80.2% purity in LCMS at 220 nm) as yellow solid. (ESI⁺) m/z: 528.1 (M+H)⁺, (C₂₉H₄IN₃O₆).

B. tert-Butyl 5-amino-4-(5-(2-(4,4-dimethylcyclohexyl)-1-methyl-1H-imidazol-4-yl)-1-oxoisoindolin-2-yl)-5-oxopentanoate: To a solution of tert-butyl 5-amino-4-(5-(N-(4, 4-dimethylcyclohexane-1-carbonyl)-N-methylglycyl)-1-oxoisoindolin-2-yl)-5-oxopentanoate (300 mg, 568 μmol, 1.00 eq) in HCONH₂ (4.00 mL) was added NH₄OAc (307 mg, 3.98 mmol, 7.00 eq). Then the mixture was stirred at 100° C. for 12 h. The mixture was poured into H₂O (20.0 mL), and extracted with Ethyl acetate (3×30.0 mL). The combined organic layer was dried over Na₂SO₄ and concentrated under reduced pressure to get residue. The residue was purified by preparative-TLC (Dichloromethane:Methanol=10:1, R_{f}=0.30) to give the title compound (120 mg, 236 μmol, 41.4% yield) as yellow oil. (ESI⁺) m/z: 509.3 (M+H)⁺, (C₂₉H₄₀N₄O₄).

C. 3-(5-(2-(4,4-Dimethylcyclohexyl)-1-methyl-1H-imidazol-4-yl)-1-oxoisoindolin-2-yl)piperidine-2,6-dione: To a solution of tert-butyl 5-amino-4-(5-(2-(4,4-dimethylcyclohexyl)-1-methyl-1H-imidazol-4-yl)-1-oxoisoindolin-2-yl)-5-oxopentanoate (120 mg, 236 μmol, 1.00 eq) in ACN (3.00 mL) was added TsOH (163 mg, 944 μmol, 4.00 eq). Then the mixture was stirred at 80° C. for 3 h. The reaction mixture was concentrated under reduced pressure to get a residue at 45° C. The residue was purified by preparative-HPLC (using a Phenomenex luna C18 (150×25 mm×10 μm) and gradient of 25.0%-40.0% acetonitrile in water containing 0.50% TFA over 10 min at a flow rate of 25 mL/min) to give the title compound (70.2 mg, 156 μmol, 66.0% yield, 96.2% purity in HPLC at 220 nm) as white solid. ¹H NMR: (400 MHz, DMSO-d₆) δ 11.02 (s, 1H), 8.20-8.08 (m, 1H), 8.03-8.01 (m, 1H), 7.92-7.85 (m, 2H), 5.16-5.11 (m, 1H), 4.55-4.38 (m, 2H), 3.87-3.78 (m, 3H), 3.23-3.14 (m, 1H), 2.94-2.89 (m, 1H), 2.63-2.59 (m, 1H), 2.45-2.35 (m, 1H), 2.10-2.04 (m, 1H), 1.95-1.82 (m, 6H), 1.25-1.23 (m, 2H), 1.04 (s, 3H), 0.94 (s, 3H). (ESI⁺) m/z: 435.2 (M+H)⁺, (C₂₅H₃₀N₄O₃).

### Example 540

### Synthesis of 3-(5-(2-(difluoromethyl)-1-methyl-5-(pyrimidin-5-yl)-1H-imidazol-4-yl)-1-oxoisoindolin-2-yl)piperidine-2,6-dione

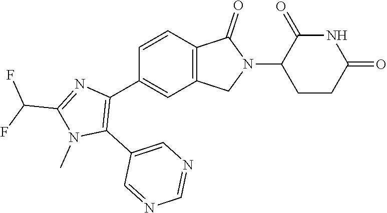 [SMILES: Cn1c(C(F)F)nc(-c2ccc3c(c2)CN(C2CCC(=O)NC2=O)C3=O)c1-c1cncnc1]

A. 2-(Difluoromethyl)-1-methyl-1H-imidazole: A mixture of 1-methylimidazole (8.00 g, 97.4 mmol, 7.77 mL, 1.00 eq), trimethyl(trifluoromethyl)silane (41.5 g, 292 mmol, 43.2 mL, 3.00 eq), TBAT (5.26 g, 9.74 mmol, 0.10 eq) in THF (800 mL) was degassed and purged with N₂ for 3 times at −50° C., and then the mixture was stirred at 25° C. for 16 h under N₂ atmosphere. The reaction mixture was concentrated under reduced pressure to remove THF. The residue was purified by column chromatography (SiO₂, PE/EA=50/1 to 1/1, TLC: PE/EA=1/1, R_{f}=0.50) to give the title compound (6.50 g, 49.2 mmol, 50.4% yield) as a black brown oil. ¹H NMR: (400 MHz, CDCl₃) δ 7.02 (S, 1H), 6.95 (s 1H), 6.86-6.60 (m, 1H), 3.82 (s, 3H).

B. 5-(2-(Difluoromethyl)-1-methyl-1H-imidazol-5-yl)pyrimidine: A mixture of 2-(difluoromethyl)-1-methyl-imidazole (3.00 g, 22.7 mmol, 1.00 eq), 5-bromopyrimidine (4.33 g, 27.2 mmol, 1.20 eq), Pd(OAc)₂ (509 mg, 2.27 mmol, 0.10 eq), P(oxole)₃ (527 mg, 2.27 mmol, 0.10 eq), K₂CO₃ (6.28 g, 45.4 mmol, 2.00 eq) in DMF (60.0 mL) was degassed and purged with N₂ for 3 times, and then the mixture was stirred at 100° C. for 16 h under N₂ atmosphere. The mixture was filtered, and concentrate under vacuum to get residue. The residue was purified by column chromatography (SiO₂, DCM/MeOH=50/1 to 20/1, TLC: DCM/MeOH=20/1, R_{f}=0.40) to give the title compound (1.60 g, 7.46 mmol, 32.8% yield, 98.1% purity in LCMS at 220 nm) as light yellow solid. ¹HNMR: (400 MHz, DMSO-d₆) δ 9.26 (s, 1H), 9.25 (s, 2H), 7.38-7.33 (m, 1H), 7.23-7.10 (m, 1H), 3.79 (s, 3H). (ESI⁺) m/z: 211.0 (M+H)⁺, (C₉H₈N₄F₂).

C. 5-(4-Bromo-2-(difluoromethyl)-1-methyl-1H-imidazol-5-yl)pyrimidine: A mixture of 5-[2-(difluoromethyl)-3-methyl-imidazol-4-yl]pyrimidine (600 mg, 2.85 mmol, 1.00 eq), NBS (533 mg, 3.00 mmol, 1.05 eq) in ACN (6.00 mL) was degassed and purged with N₂ for 3 times at 0° C., and then the mixture was stirred at 25° C. for 2 h under N₂ atmosphere. The reaction mixture was diluted with water 10.0 mL and extracted with EA 15.0 mL (3×5.00 mL). The combined organic layers were washed with brine 30.0 mL (2×15.0 mL), dried over Na₂SO₄, filtered and concentrated under reduced pressure to give a residue to give the title compound (780 mg, 2.70 mmol, 94.5% yield, 100% purity in LCMS at 220 nm) as light yellow solid. ¹H NMR: (400 MHz, DMSO-d₆) δ9.38 (s, 1H), 9.02 (s, 2H), 7.45-7.19 (m, 1H), 3.71 (s, 3H). (ESI⁺) m/z: 289.0 (M+H)⁺, (C₉H₇N₄F₂Br).

D. 3-(5-(2-(Difluoromethyl)-1-methyl-5-(pyrimidin-5-yl)-1H-imidazol-4-yl)-1-oxoisoindolin-2-yl)piperidine-2,6-dione: A mixture of 5-[5-bromo-2-(difluoromethyl)-3-methyl-imidazol-4-yl]pyrimidine (780 mg, 2.70 mmol, 1.00 eq), 3-[1-oxo-5-(4,4,5,5-tetramethyl-1,3,2-dioxaborolan-2-yl)isoindolin-2-yl]piperidine-2,6-dione (3.00 g, 8.09 mmol, 3.00 eq), K₃PO₄ (1.15 g, 5.40 mmol, 2.00 eq), Ru-Phos-Pd-G₃ (225 mg, 269 μmol, 0.10 eq) in dioxane (24.0 mL), H₂O (1.20 mL) was degassed and purged with N₂ for 3 times, and then the mixture was stirred at 100° C. for 1 h under N₂ atmosphere. The reaction mixture was filtered and concentrated under reduced pressure to give a residue. The filtrate was purified by preparative-HPLC (using a Phenomenex luna C18 (250 mm×70 mm×10 μm) and gradient of 5-40% acetonitrile in water containing 1% FA over 30 min at a flow rate of 140 mL/min) to give the title compound (664 mg, 1.46 mmol, 54.0% yield, 99.3% purity in HPLC at 220 nm) as light yellow solid. 1H NMR: (400 MHz, DMSO-d₆) δ 10.9 (s, 1H), 9.34 (s, 1H), 8.95 (s, 2H), 7.63-7.61 (m, 2H), 7.47-7.21 (m, 2H), 5.25-5.14 (m, 1H), 4.42-4.24 (m, 2H), 3.65 (s, 3H), 2.96-2.85 (m, 1H), 2.60-2.50 (m, 1H), 2.38-2.30 (m, 1H), 2.00-1.97 (m, 1H). (ESI⁺) m/z: 453.0 (M+H)⁺, (C₂₂H₁₈N₆F₂₀₃).

### Example 541

### Synthesis of 3-(5-(5-(4-(1,1-difluoroethyl)phenyl)-1-methyl-1H-pyrazol-4-yl)-1-oxoisoindolin-2-yl)piperidine-2,6-dione

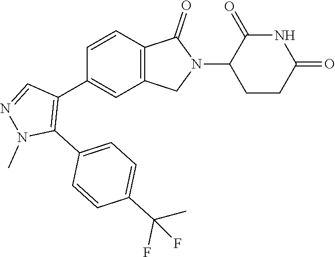 [SMILES: Cn1ncc(-c2ccc3c(c2)CN(C2CCC(=O)NC2=O)C3=O)c1-c1ccc(C(C)(F)F)cc1]

A. tert-Butyl 5-amino-4-(5-(5-(4-(1,1-difluoroethyl)phenyl)-1-methyl-1H-pyrazol-4-yl)-1-oxoisoindolin-2-yl)-5-oxopentanoate: To a solution of tert-butyl 5-amino-4-[5-(5-bromo-1-methyl-pyrazol-4-yl)-1-oxo-isoindolin-2-yl]-5-oxo-pentanoate (300 mg, 628 μmol, 1.00 eq) and 2-[4-(1,1-difluoroethyl)phenyl]-4,4,5,5-tetramethyl-1,3,2-dioxaborolane (252 mg, 942 μmol, 1.50 eq) in dioxane (6.00 mL) and H₂O (1.50 mL) was added ditert-butyl(cyclopentyl)phosphane; dichloropalladium; iron (40.9 mg, 62.8 μmol, 0.10 eq) and K₃PO₄ (400 mg, 1.89 mmol, 3.00 eq) at 20° C. The mixture was stirred at 65° C. for 16 h. The mixture was poured into H₂O (10.0 mL) extracted with ethyl acetate (2×30.0 mL) to collect the organic layers, dried over anhydrous Na₂SO₄, filtered and the filtrate was concentrated under reduced pressure to get a residue. The residue was purified by preparative-TLC (DCM/MeOH=20/1, R_{f}=0.30) to give the compound (280 mg, 436 μmol, 69.4% yield, 84% purity in LCMS at 220 nm) as a yellow solid. ¹H NMR: (400 MHz, CDCl₃) δ 8.02 (s, 1H), 7.67-7.63 (m, 4H), 7.49 (d, J=8.4 Hz, 1H), 7.41 (d, J=8.0 Hz, 2H), 6.35 (s, 1H), 5.32-5.31 (m, 1H), 4.89-4.85 (m, 1H), 4.44-4.33 (m, 2H), 3.54 (s, 3H), 2.41-2.15 (m, 4H), 2.01 (t, J=18.0 Hz, 3H), 1.43-1.42 (m, 9H). (ESI⁺) m/z: 539.1 (M+H)⁺, (C₂₉H₃₂N₄O₄F₂).

B. 3-(5-(5-(4-(1,1-difluoroethyl)phenyl)-1-methyl-1H-pyrazol-4-yl)-1-oxoisoindolin-2-yl)piperidine-2,6-dione: To a solution of tert-butyl 5-amino-4-[5-[5-[4-(1,1-difluoroethyl)phenyl]-1-methyl-pyrazol-4-yl]-1-oxo-isoindolin-2-yl]-5-oxo-pentanoate (150 mg, 233 μmol, 1.00 eq) in ACN (1.50 mL) was added TsOH (80.5 mg, 467 μmol, 2.00 eq) at 20° C. The mixture was stirred at 80° C. for 4 h. The mixture was poured in H₂O (20.0 mL) and extracted with ethyl acetate (3×20.0 mL) to collect the organic layer. The organic layers were dried over Na₂SO₄, filtered and the filtrate was concentrated under vacuum at 45° C. to get a residue. The residue was purified by preparative-TLC (DCM/MeOH=15/1, R_{f}=0.30) to give the residue. The residue was purified by preparative-HPLC (using a Phenomenex luna C18 (150×25 mm×10 μm) and gradient of 7%-37% acetonitrile in water containing 0.05% FA over 13 min at a flow rate of 25 mL/min) to give the title compound (15.0 mg, 31.5 μmol, 13.5% yield, 97.7% purity in HPLC at 220 nm) as a yellow solid. ¹H NMR: (400 MHz, DMSO-d₆) δ 11.0 (s, 1H), 7.89 (s, 1H), 7.00 (d, J=8.0 Hz, 2H), 7.62 (s, 1H), 7.56-7.53 (m, 3H), 7.41 (d, J₁=8.4 Hz, 1H), 5.07 (dd, J₁=4.4 Hz, J₂=13.2 Hz, 1H), 4.39-4.20 (m, 2H), 3.51 (s, 3H), 2.94-2.85 (m, 1H), 2.67-2.58 (m, 1H), 2.43-2.32 (m, 1H), 2.08-1.96 (m, 4H). (ESI⁺) m/z: 465.2 (M+H)⁺, (C₂₅H₂₂N₄O₃F₂).

### Example 542

### Synthesis of 3-(5-(5-(2,2-difluorobenzo[d][1,3]dioxol-5-yl)-1-methyl-1H-pyrazol-4-yl)-1-oxoisoindolin-2-yl)piperidine-2,6-dione

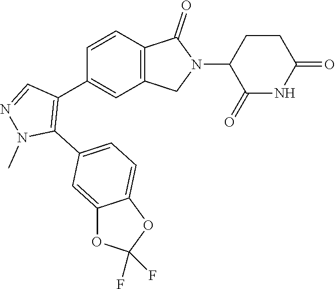 [SMILES: Cn1ncc(-c2ccc3c(c2)CN(C2CCC(=O)NC2=O)C3=O)c1-c1ccc2c(c1)OC(F)(F)O2]

A. tert-Butyl 5-amino-4-(5-(5-(2,2-difluorobenzo[d][1,3]dioxol-5-yl)-1-methyl-1H-pyrazol-4-yl)-1-oxoisoindolin-2-yl)-5-oxopentanoate: To a solution of (2,2-difluorobenzo[d][1,3]dioxol-5-yl)boronic acid (159 mg, 785 μmol, 1.50 eq), tert-butyl5-amino-4-(5-(5-bromo-1-methyl-1H-pyrazol-4-yl)-1-oxoisoindolin-2-yl)-5-oxopentanoate (250 mg, 523 μmol, 1.00 eq) and K₃PO₄ (333 mg, 1.57 mmol, 3.00 eq) in dioxane (10.0 mL) and H₂O (2.50 mL) was added Pd(dtbpf)Cl₂ (34.1 mg, 52.3 μmol, 0.10 eq) under N₂. The mixture was stirred at 65° C. for 16 h under N₂. The mixture was filtered to collect liquid and concentrated under reduced pressure to get residue. The residue was purified by column chromatography (SiO₂, Dichloromethane:Methanol=10:1, R_{f}=0.25) to get the title compound (450 mg, 765 μmol, 73.0% yield, 94.3% purity at 220 nm in LCMS) as yellow solid. ¹H NMR: (400 MHz, DMSO-d₆) δ 7.86 (s, 1H), 7.65-7.52 (m, 5H), 7.51-7.43 (m, 1H), 7.22-7.17 (m, 2H), 4.70-4.67 (m, 1H), 4.54-4.34 (m, 2H), 3.48 (s, 3H), 2.21-1.90 (m, 4H), 1.33 (m, 9H). (ESI⁺) m/z: 555.2 (M+H)⁺, (C₂₈H₂₈F₂N₄O₆).

B. 3-(5-(5-(2,2-Difluorobenzo[d][1,3]dioxol-5-yl)-1-methyl-1H-pyrazol-4-yl)-1-oxoisoindolin-2-yl)piperidine-2,6-dione: To a solution of tert-butyl 5-amino-4-(5-(5-(2,2-difluorobenzo[d][1,3]dioxol-5-yl)-1-methyl-1H-pyrazol-4-yl)-1-oxoisoindolin-2-yl)-5-oxopentanoate (200 mg, 360 μmol, 1.00 eq) in ACN (10.0 mL) was added TsOH (248 mg, 1.44 mmol, 4.00 eq). The mixture was stirred at 70° C. for 4 h. The mixture was filtered to collect liquid and concentrated under reduced pressure to get residue. The crude product was purified by preparative-HPLC (using a Phenomenex luna C18 (150 mm×25 mm 10 μm) and gradient of 12-42% acetonitrile in water containing 0.05% FA over 15 min at a flow rate of 25 mL/min) to give the title compound (82.2 mg, 163 μmol, 47.7% yield, 95.7% purity in HPLC at 220 nm) as white solid. ¹H NMR: (400 MHz, DMSO-d₆) δ 10.9 (s, 1H), 7.87 (s, 1H), 7.62-7.59 (m, 1H), 7.57-7.52 (m, 3H), 7.50-7.47 (m, 1H), 7.22-7.20 (m, 1H), 5.11-5.03 (m, 1H), 4.39-4.20 (m, 2H), 3.49 (s, 3H), 3.03-3.00 (m, 2H), 2.94-2.85 (m, 1H), 2.60-2.55 (m, 1H), 2.37-2.33 (m, 1H), 1.98-1.96 (m, 1H). (ESI⁺) m/z: 481.0 (M+H)⁺, (C₂₄H₁₈F₂N₄O₅).

### Example 543

### Synthesis of 3-[5-(2-isopropyl-1-methyl-5-pyrimidin-5-yl-imidazol-4-yl)-1-oxo-isoindolin-2-yl]piperidine-2,6-dione

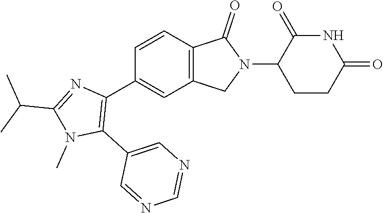 [SMILES: CC(C)c1nc(-c2ccc3c(c2)CN(C2CCC(=O)NC2=O)C3=O)c(-c2cncnc2)n1C]

A. 2-Isopropyl-1-methyl-imidazole: To a solution of 2-isopropyl-1H-imidazole (5.00 g, 45.3 mmol, 1.00 eq) in toluene (55.0 mL) was added NaH (2.18 g, 54.4 mmol, 60% purity, 1.20 eq) at 0-5° C. under N₂. After stirring at 20° C. for 1 h, the mixture was cooled to 0-5° C., CH₃I (9.66 g, 68.0 mmol, 4.24 mL, 1.50 eq) was added to the mixture, then the mixture was stirred at 20° C. for 3 h. The reaction mixture was poured into NH₄Cl (100 mL) and extracted with EA 240 mL (80 mL×3). The combined layer was dried over anhydrous Na₂SO₄, filtered and concentrated under reduced pressure to give a residue. The residue was purified by column chromatography (SiO₂, DCM:MeOH=100/1 to 2/1, DCM:MeOH=2/1, R_{f}=0.50) to give the title compound (4.50 g, 36.2 mmol, 79.8% yield) as a white solid. ¹H NMR: (400 MHz, CDCl₃-d₆) δ 6.87 (s, 1H), 6.65 (s, 1H), 3.48 (s, 3H), 2.95-2.84 (m, 3H), 1.22 (s, 6H). (ESI⁺) m/z: 124.1 (M+H)⁺, (C₇H₁₂N₂).

B. 5-(2-Isopropyl-3-methyl-imidazol-4-yl)pyrimidine: To a solution of 2-isopropyl-1-methyl-imidazole (4.50 g, 36.2 mmol, 1.00 eq), 5-bromopyrimidine (5.76 g, 36.2 mmol, 1.00 eq), K₂CO₃ (10.0 g, 72.4 mmol, 2.00 eq) in DMF (100 mL) was added Pd(OAc)₂ (813 mg, 3.62 mmol, 0.10 eq) and tris(2-furyl)phosphane (841 mg, 3.62 mmol, 0.10 eq), then the reaction mixture was stirred at 110° C. for 10 h under N₂. The reaction mixture was concentrated under vacuum. The residue was purified by column chromatography (SiO₂, PE: EA=100/1 to 2/1, PE: EA=10/1, R_{f}=0.25) to give the title compound (3.00 g, 12.9 mmol, 35.6% yield, 87.0% purity in LCMS at 220 nm) as a brown solid. ¹H NMR: (400 MHz, DMSO-d₆) δ 9.15 (s, 1H), 8.94 (s, 2H), 7.14 (s, 1H), 3.62 (s, 3H), 3.18-3.11 (m, 1H), 1.25 (s, 6H). (ESI⁺) m/z: 203.0 (M+H)⁺, (C₁₁H₁₄N₄).

C. 5-(5-Bromo-2-isopropyl-3-methyl-imidazol-4-yl)pyrimidine: To a solution of 5-(2-isopropyl-3-methyl-imidazol-4-yl)pyrimidine (3.00 g, 12.9 mmol, 1.00 eq) in ACN (30.0 mL) was added NBS (2.07 g, 11.6 mmol, 0.90 eq), the mixture was stirred at 25° C. for 2 h. The reaction mixture was poured into H₂O (50.0 mL) and extracted with EA (3×50.0 mL). The combined layer was dried over anhydrous Na₂SO₄, filtered and concentrated under reduced pressure to give a residue. The residue was purified by column chromatography (SiO₂, PE: EA=100/1 to 2/1, PE: EA=2/1, R_{f}=0.60) to give the title compound (2.20 g, 7.82 mmol, 60.6% yield, >99% purity in LCMS at 220 nm) as a white solid. ¹H NMR: (400 MHz, CDCl₃-d₆) δ 9.25 (s, 1H), 8.82 (s, 2H), 3.54 (s, 3H), 3.11-3.04 (m, 1H), 1.40 (s, 6H). (ESI⁺) m/z: 280.8 (M+H)⁺, (C₁₁H₁₃BrN₄).

D. 3-[5-(2-Isopropyl-1-methyl-5-pyrimidin-5-yl-imidazol-4-yl)-1-oxo-isoindolin-2-yl]piperidine-2,6-dione: To a solution of 5-(5-bromo-2-isopropyl-3-methyl-imidazol-4-yl)pyrimidine (500 mg, 1.78 mmol, 1.00 eq), 3-[1-oxo-5-(4,4,5,5-tetramethyl-1,3,2-dioxaborolan-2-yl)isoindolin-2-yl]piperidine-2,6-dione (1.98 g, 5.34 mmol, 3.00 eq), K₃PO₄ (754 mg, 3.56 mmol, 2.00 eq) in dioxane (20.0 mL) and H₂O (1.00 mL) was added RuPhos Pd G₃ (148 mg, 177 μmol, 0.10 eq). Then the mixture was stirred at 100° C. for 2 h. The reaction mixture was concentrated under reduced pressure. The crude product was purified by preparative-HPLC (using a Phenomenex luna C18 (250 mm×70 mm, 15 m) and gradient of 1%-25% acetonitrile in water containing 0.05% FA over 18 min at a flow rate of 25 mL/min) to give the title compound (419 mg, 940 μmol, 52.9% yield, 99.7% purity in HPLC at 220 nm) as a white solid. ¹H NMR: (400 MHz, DMSO-d₆) δ 10.98 (s, 1H), 9.28 (s, 1H), 8.87 (s, 2H), 7.58 (d, J=12 Hz, 2H), 7.39 (d, J=8 Hz, 2H), 5.10-5.05 (m, 1H), 4.40-4.22 (m, 2H), 3.47 (s, 3H), 3.25-3.18 (m, 1H), 2.94-2.85 (m, 1H), 2.58 (d, J=16 Hz, 1H), 2.42-2.29 (m, 1H), 2.00-1.95 (m, 1H), 1.32 (d, J=6.8 Hz, 6H). (ESI⁺) m/z: 445.1 (M+H)⁺, (C₂₄H₂₄N₆O₃).

### Example 544

### Synthesis of 3-(5-(1, 2-dimethyl-5-(pyrimidin-5-yl)-1H-imidazol-4-yl)-1-oxoisoindolin-2-yl)piperidine-2, 6-dione

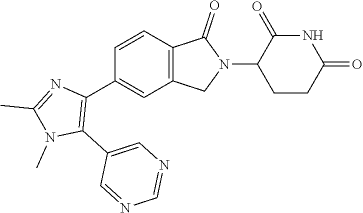 [SMILES: Cc1nc(-c2ccc3c(c2)CN(C2CCC(=O)NC2=O)C3=O)c(-c2cncnc2)n1C]

A. 5-(1, 2-Dimethyl-1H-imidazol-5-yl)pyrimidine: A mixture of 1, 2-dimethyl-1H-imidazole (1.00 g, 10.4 mmol, 1.00 eq), 5-bromopyrimidine (4.96 g, 31.2 mmol, 3.00 eq), K₂CO₃ (2.88 g, 20.8 mmol, 2.00 eq), Pd(OAc)₂ (233 mg, 1.04 mmol, 0.10 eq) and P(oxole)₃ (241 mg, 1.04 mmol, 0.10 eq) in DMF (40.0 mL) was degassed and purged with N₂ for 3 times, and then the mixture was stirred at 110° C. for 16 h under N₂ atmosphere. The mixture was concentrated under vacuum to get residue. The residue was purified by column chromatography (SiO₂, DCM/MeOH=30/1 to 10/1, TLC: DCM/MeOH=10:1, R_{f}=0.30) to give the title compound (1.30 g, 7.46 mmol, 71.7% yield) as a light yellow solid. ¹H NMR: (400 MHz, CDCl₃) δ 9.20 (s, 1H), 8.78 (s, 2H), 7.12 (s, 1H), 3.59 (s, 3H), 2.50 (s, 3H).

B. 5-(4-Bromo-1,2-dimethyl-1H-imidazol-5-yl)pyrimidine: A mixture of 5-(1, 2-dimethyl-1H-imidazol-5-yl)pyrimidine (1.00 g, 5.74 mmol, 1.00 eq), NBS (919 mg, 5.17 mmol, 0.90 eq) in ACN (10.0 mL) was degassed and purged with N₂ for 3 times at 0° C., and then the mixture was stirred at 25° C. for 2 h under N₂ atmosphere. The reaction mixture was filtered, and then diluted with water 20.0 mL and extracted with DCM 30.0 mL (3×10.0 mL). The combined organic layers were washed with brine 60.0 mL (2×30.0 mL), dried over Na₂SO₄, filtered and concentrated under reduced pressure to give a residue. The residue was purified by column chromatography (SiO₂, DCM/MeOH=50/1 to 20/1, TLC: DCM/MeOH=20:1, R_{f}=0.48) to give the title compound (920 mg, 3.63 mmol, 62.3% yield) as a light yellow solid. ¹H NMR: (400 MHz, CDCl₃) δ 9.24 (s, 1H), 8.80 (s, 2H), 3.51 (s, 3H), 2.47 (s, 3H).

C. 3-(5-(1,2-Dimethyl-5-(pyrimidin-5-yl)-1H-imidazol-4-yl)-1-oxoisoindolin-2-yl)piperidine-2,6-dione: A mixture of 5-(4-bromo-1, 2-dimethyl-1H-imidazol-5-yl)pyrimidine (300 mg, 1.19 mmol, 1.00 eq), 3-(1-oxo-5-(4, 4, 5, 5-tetramethyl-1, 3, 2-dioxaborolan-2-yl)isoindolin-2-yl)piperidine-2, 6-dione (1.32 g, 3.56 mmol, 3.00 eq), K₃PO₄ (503 mg, 2.37 mmol, 2.00 eq), Ru-Phos-Pd-G₃ (99.1 mg, 118 μmol, 0.10 eq) in dioxane (10.0 mL), H₂O (1.00 mL) was degassed and purged with N₂ for 3 times and then the mixture was stirred at 100° C. for 2 h under N₂ atmosphere. The reaction mixture was filtered and concentrated under reduced pressure to give a residue. The filtrate was purified by preparative-HPLC (using a Phenomenex luna C18 (200 mm×40 mm×10 μm) and gradient of 1-20% acetonitrile in water containing 1% TFA over 15 min at a flow rate of 75 mL/min) to give the title compound (32.0 mg, 73.8 μmol, 6.23% yield, 96.1% purity in HPLC at 220 nm) as a white solid. ¹H NMR: (400 MHz, MeOD) δ 9.34 (s, 1H), 8.91 (s, 2H), 7.85-7.83 (m, 1H), 7.58 (s, 1H), 7.52-7.50 (m, 1H), 5.17-5.13 (m, 1H), 4.53-4.42 (m, 2H), 3.71 (s, 3H), 2.94-2.85 (m, 1H), 2.81-2.74 (m, 4H), 2.52-2.41 (m, 1H), 2.19-2.13 (m, 1H). (ESI⁺) m/z: 417.1 (M+H)⁺ (C₂₂H₂N₆O₃).

### Example 545

### Synthesis of 3-(5-(2-(4,4-difluorocyclohexyl)-1-methyl-1H-imidazol-4-yl)-1-oxoisoindolin-2-yl)piperidine-2,6-dione

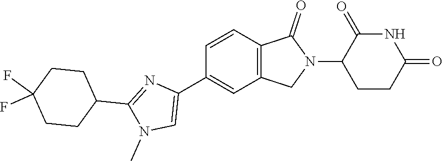 [SMILES: Cn1cc(-c2ccc3c(c2)CN(C2CCC(=O)NC2=O)C3=O)nc1C1CCC(F)(F)CC1]

A. N-(2,2-Dimethoxyethyl)-4,4-difluoro-N-methylcyclohexane-1-carboxamide: To a solution of 4,4-difluorocyclohexanecarboxylic acid (5.00 g, 30.4 mmol, 1.00 eq), 2,2-dimethoxy-N-methylethanamine (4.36 g, 36.5 mmol, 4.70 mL, 1.20 eq) and HOBt (4.94 g, 36.5 mmol, 1.20 eq) in DMF (50.0 mL) was added EDCI (7.01 g, 36.5 mmol, 1.20 eq) at 25° C. The reaction mixture was stirred at 25° C. for 12 h under N₂. After the reaction was completed, the reaction mixture was filtered and the filtrate was concentrated in vacuum to give a residue. The residue was dissolved in water (10.0 mL) and ethyl acetate (10.0 mL), then the residue was extracted with ethyl acetate (3×15.0 mL), dried over Na₂SO₄, filtered and the filtrate was concentrated in vacuum to give the title compound (10.6 g, crude) as colorless oil. (ESI⁺) m/z: 266.1 (M+H)⁺, (C₁₂H₂₁F₂NO₃).

B. 2-(4,4-Difluorocyclohexyl)-1-methyl-1H-imidazole: A solution of N-(2,2-dimethoxyethyl)-4,4-difluoro-N-methyl-cyclohexanecarboxamide (5.00 g, 18.8 mmol, 1.00 eq) and NH₄OAc (29.0 g, 376 mmol, 20.0 eq) in AcOH (100 mL) was stirred at 125° C. for 12 h under N₂. After the reaction was completed, the reaction mixture was poured into water (100 mL) and neutralized with 10 N NaOH (100 mL) to pH=7. Then the mixture was extracted with dichloromethane (3×100 mL). The combined organic layer was washed with brine (3×50.0 mL), dried over Na₂SO₄, filtered and the filtrate was concentrated in vacuum to get a residue. The residue was purified by column chromatography (SiO₂, Dichloromethane:Methanol=100:1 to 20:1, R_{f}=0.40 (Dichloromethane:Methanol=20:1)) to give the title compound (600 mg, 2.78 mmol, 14.7% yield, 92.8% purity in LCMS at 220 nm) as yellow oil. ¹H NMR: (400 MHz, CDCl₃) δ 7.00 (s, 1H), 6.79 (s, 1H), 3.70-3.53 (m, 1H), 3.63 (s, 3H), 2.81-2.77 (m, 1H), 2.28-2.24 (m, 2H), 2.03-1.98 (m, 3H), 1.88-1.84 (m, 2H). (ESI⁺) m/z: 201.1 (M+H)⁺, (C₁₀H₁₄F₂N₂).

C. 4,5-Dibromo-2-(4,4-difluorocyclohexyl)-1-methyl-1H-imidazole: To a solution of 2-(4,4-difluorocyclohexyl)-1-methyl-imidazole (100 mg, 489 μmol, 1.00 eq) in THF (2.00 mL) was added NBS (191 mg, 1.08 mmol, 2.20 eq) at 0° C. The mixture was stirred at 60° C. for 12 h under N₂. After the reaction was completed, the mixture was poured into 15 mL of saturated NaCl solution. The aqueous layer was extracted with ethyl acetate (3×20.0 mL) and the combined organic layer was washed with brine (3×20.0 mL), dried over Na₂SO₄, filtered and the filtrate was concentrated in vacuum to get a residue. The residue was purified by column chromatography (SiO₂, Petroleum ether:Ethyl acetate=100:1 to 3:1, R_{f}=0.50 (Petroleum ether:Ethyl acetate=3:1)) to give the title compound (300 mg, 806 μmol, 54.9% yield, 96.3% purity in LCMS at 220 nm) as a yellow solid. ¹H NMR: (400 MHz, DMSO-d₆) δ 3.58 (s, 3H), 2.98-2.92 (m, 1H), 2.11-2.05 (m, 2H), 1.94-1.87 (m, 4H), 1.72-1.62 (m, 2H). (ESI⁺) m/z: 356.9 (M+H)⁺, (C₁₀H₁₂Br₂F₂N₂).

D. 4-Bromo-2-(4,4-difluorocyclohexyl)-1-methyl-1H-imidazole: To a solution of 4,5-dibromo-2-(4,4-difluorocyclohexyl)-1-methyl-imidazole (250 mg, 672 μmol, 1.00 eq) in THF (5.00 mL) was added EtMgBr (3 M, 1.34 mL, 6.00 eq) at 0° C. under N₂. The mixture was stirred at 25° C. for 2 h under N₂. After the reaction was completed, the reaction mixture was quenched with saturated NH₄Cl solution (10.0 mL) and extracted with ethyl acetate (3×15.0 mL). The combined organic layer was washed with brine (3×15.0 mL), dried over Na₂SO₄, filtered and the filtrate was concentrated in vacuum to get a residue. The residue was purified by preparative-TLC ((Petroleum ether:Ethyl acetate=1:1,R_{f}=0.50) to give the title compound (110 mg, 373 μmol, 55.6% yield, 94.9% purity in LCMS at 220 nm) as yellow oil. ¹H NMR: (400 MHz, DMSO-d₆) δ 7.15 (s, 1H), 3.58 (s, 3H), 2.98-2.92 (m, 1H), 2.10-2.08 (m, 2H), 1.94-1.85 (m, 4H), 1.70-1.66 (m, 2H). (ESI⁺) m/z: 279.0 (M+H)⁺, (C₁₀H₁₃BrF₂N₂).

E. 3-(5-(2-(4,4-Difluorocyclohexyl)-1-methyl-1H-imidazol-4-yl)-1-oxoisoindolin-2-yl)piperidine-2,6-dione: To a solution of 4-bromo-2-(4,4-difluorocyclohexyl)-1-methyl-imidazole (100 mg, 339 μmol, 1.00 eq) in dioxane (3.00 mL) and H₂O (0.15 mL) was added 3-[1-oxo-5-(4,4,5,5-tetramethyl-1,3,2-dioxaborolan-2-yl)isoindolin-2-yl]piperidine-2,6-dione (314 mg, 849 μmol, 2.50 eq), Ru-Phos-Pd-G₃ (28.4 mg, 34.0 μmol, 0.10 eq) and K₃PO₄ (144 mg, 679 μmol, 2.00 eq) under N₂. The mixture was stirred at 100° C. for 2 h under N₂. After the reaction was completed, the reaction mixture was filtered and the filtrate was concentrated under vacuum to give a residue. The residue was purified by preparative-TLC (Dichloromethane:Methanol=10:1, R_{f}=0.50) and by preparative-HPLC (using a Phenomenex Luna C18 (150 mm×25 mm×5 μm) and gradient of 21-51% acetonitrile in water containing 0.5% NH₄HCO₃ over 10 min at a flow rate of 25.0 mL/min) to give the title compound (11.1 mg, 24.8 μmol, 7.38% yield, 99.0% purity in HPLC at 220 nm) as an off-white solid. ¹H NMR: (400 MHz, CD₃₀D) δ 7.93 (s, 1H), 7.85 (d, J=8.4 Hz, 1H), 7.76 (d, J=8.0 Hz, 1H), 7.48 (s, 1H), 5.15 (dd, J=13.2, 5.2 Hz, 1H), 4.51 (d, J=7.2 Hz, 2H), 3.73 (s, 3H), 3.04-3.02 (m, 2H), 3.01-3.00 (m, 1H), 2.92-2.81 (m, 1H), 2.23-2.17 (m, 3H), 2.03-1.95 (m, 6H). (ESI⁺) m/z: 443.1 (M+H)⁺, (C₂₃H₂₄F₂N₄O₃).

### Example 546

### Synthesis of 3-(5-(5-(4,4-difluorocyclohexyl)-1-methyl-1H-pyrazol-3-yl)-1-oxoisoindolin-2-yl)piperidine-2,6-dione

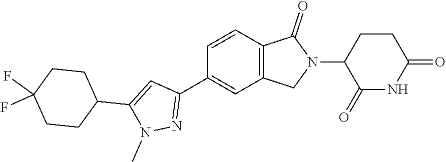 [SMILES: Cn1nc(-c2ccc3c(c2)CN(C2CCC(=O)NC2=O)C3=O)cc1C1CCC(F)(F)CC1]

A. 3-(4,4-Difluorocyclohexyl)-3-oxopropanenitrile: To a solution of ethyl 4,4-difluorocyclohexane-1-carboxylate (500 mg, 2.60 mmol, 1.00 eq), ACN (533 mg, 13.0 mmol, 684 μL, 5.00 eq) in THF (12.0 mL) was added t-BuOK (583 mg, 5.20 mmol, 2.00 eq) dropwise at 0° C. Then the mixture was stirred at 25° C. for 2 h under N₂. The mixture poured into H₂O (40.0 mL), the resulting solution was adjusted to pH=7 with HCl aqueous (0.50 M, 5.00 mL) and the resulting solution was extracted with Ethyl acetate (2×50.0 mL), the combined organic layer was washed with saturated NaCl aqueous (2×60.0 mL), dried over Na₂SO₄, and concentrated under reduced pressure to get residue to get the title compound (400 mg, crude) as brown solid. ¹H NMR: (400 MHz, DMSO-d₆) δ 4.21 (s, 2H), 2.64-2.61 (m, 1H), 2.05-1.86 (m, 6H), 1.51-1.48 (m, 2H), (ESI⁺) m/z: 187.01 (M+H)⁺, (C₉H₁₁F₂NO).

B. 5-(1H-Pyrazol-5-yl) pyrimidine: To a solution of (E)-3-(dimethylamino)-1-(pyrimidin-5-yl) prop-2-en-1-one (3.50 g, 19.7 mmol, 1.00 eq) in EtOH (70.0 mL) was added N₂H₄·H₂O (3.03 g, 59.2 mmol, 2.93 mL, 98.0% purity, 3.00 eq) dropwise. The mixture was stirred at 25° C. for 2 h. The reaction mixture was poured ice cooled H₂O (100 mL) and extracted with Ethyl acetate (2×50.0 mL). The combined organic layer was washed with saturated NaCl aqueous (2×40.0 mL), dried over Na₂SO₄ and concentrated under reduced pressure to get the title compound (2.85 g, 19.3 mmol, 97.8% yield, 99.1% purity at 220 nm in LCMS) as brown solid (ESI⁺) m/z: 147.3 (M+H)⁺, (C₇H₆N₄).

C. 5-(4, 4-Difluorocyclohexyl)-3-iodo-1H-pyrazole: To a solution of 5-(4,4-difluorocyclohexyl)-1H-pyrazol-3-amine (1.00 g, 4.97 mmol, 1.00 eq), TsOH (2.14 g, 12.4 mmol, 2.50 eq) in ACN (50.0 mL) and a dropwise solution of NaNO₂ (857 mg, 12.4 mmol, 2.50 eq) in H₂O (10.0 mL) at 0° C. After 1 h later a solution of NaI (3.72 g, 24.8 mmol, 5.00 eq) in H₂O (10.0 mL) was added to the reaction mixture dropwise. Then the mixture was stirred at 25° C. for 3 h. The mixture was poured into H₂O (50.0 mL) and extracted with Ethyl acetate (2×30.0 mL). The combined organic layer was washed with saturated NaCl aqueous (2×30.0 mL), dried over Na₂SO₄ and concentrated under reduced pressure to get residue to give the title compound (1.00 g, crude) as brown solid. (ESI⁺) m/z: 313.1 (M+H)⁺, (C₉H₁₁F₂IN₂).

D. 5-(4,4-Difluorocyclohexyl)-3-iodo-1-methyl-1H-pyrazole: To a solution of 5-(4,4-difluorocyclohexyl)-3-iodo-1H-pyrazole (350 mg, 1.12 mmol, 1.00 eq) in ACN (7.00 mL) was added K₂CO₃ (172 mg, 1.24 mmol, 1.11 eq) and Mel (1.11 g, 7.85 mmol, 488 μL, 7.00 eq). The mixture was stirred at 60° C. for 12 h under N₂. The mixture was poured into H₂O (50.0 mL) and extracted with Ethyl acetate (2×25.0 mL). The combined organic layer was dried over Na₂SO₄ and concentrated under reduced pressure to get residue. The residue was purified by preparative-TLC (Petroleum ether/Ethyl acetate=1/1, R_{f}=0.60) to give the title compound (100 mg, 286 μmol, 27.3% yield, 93.4% purity at 220 nm in LCMS) as yellow oil. ¹H NMR: (400 MHz, DMSO-d₆) δ 6.30 (s, 1H), 3.77 (s, 3H), 2.92-2.86 (m, 1H), 2.07-1.89 (m, 6H), 1.54-1.50 (m, 2H) (ESI⁺) m/z: 326.9 (M+H)⁺, (C₁₀H₁₃F₂IN₂).

E. 3-(5-(5-(4,4-difluorocyclohexyl)-1-methyl-1H-pyrazol-3-yl)-1-oxoisoindolin-2-yl)piperidine-2,6-dione: To a solution of 5-(4,4-difluorocyclohexyl)-3-iodo-1-methyl-1H-pyrazole (100 mg, 306 μmol, 1.00 eq), 3-(1-oxo-5-(4,4,5,5-tetramethyl-1,3,2-dioxaborolan-2-yl)isoindolin-2-yl)piperidine-2,6-dione (454 mg, 1.23 mmol, 4.00 eq) and K₃PO₄ (130 mg, 613 μmol, 2.00 eq) in dioxane (5.00 mL) and H₂O (0.25 mL) was added Ru-Phos-Pd-G₃ (64.1 mg, 76.6 μmol, 0.25 eq) under N₂. The mixture was stirred at 100° C. for 2 h under N₂. The mixture was filtered to collect liquid and concentrated under reduced pressure to get residue. The crude product was purified by preparative-HPLC (using a Phenomenex luna C18 (150 mm×25 mm 10 μm) and gradient of 30-50% acetonitrile in water containing 0.5% TFA over 15 min at a flow rate of 25 mL/min) to give the title compound (22.4 mg, 49.5 μmol, 16.4% yield, 97.9% purity in HPLC at 220 nm) as white solid. ¹H NMR: (400 MHz, DMSO-d₆) δ 10.9 (s, 1H), 7.99 (s, 1H), 7.93-7.91 (m, 1H), 7.72-7.70 (m, 1H), 6.73 (s, 1H), 5.14-5.09 (m, 1H), 4.52-4.30 (m, 2H), 3.85 (s, 3H), 2.94-2.90 (m, 2H), 2.62-2.59 (m, 1H), 2.13-2.10 (m, 1H), 2.09-2.02 (m, 7H), 1.96-1.62 (m, 2H). (ESI⁺) m/z: 443.0 (M+H)⁺, (C₂₃H₂₄F₂N₄O₃).

### Example 547

### Synthesis of 3-(5-(2-cyclopropyl-1-methyl-5-(2-methylpyrimidin-5-yl)-1H-imidazol-4-yl)-1-oxoisoindolin-2-yl)piperidine-2,6-dione

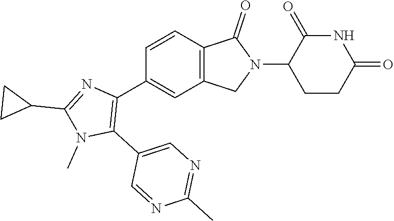 [SMILES: Cc1ncc(-c2c(-c3ccc4c(c3)CN(C3CCC(=O)NC3=O)C4=O)nc(C3CC3)n2C)cn1]

A. N-(2,2-Dimethoxyethyl)-N-methylcyclopropanecarboximidamide: To a solution of 2,2-dimethoxy-N-methylethan-1-amine (30.0 g, 251 mmol, 32.3 mL, 1.00 eq), cyclopropanecarbonitrile (21.1 g, 314 mmol, 23.1 mL, 1.25 eq) and CuCl (31.1 g, 314 mmol, 7.53 mL, 1.25 eq) at 85° C. for 16 h under N₂. The reaction mixture was concentrated under reduced pressure to give the title compound (40.0 g, crude) as brown oil. (ESI⁺) m/z: 186.2 (M+H)⁺, (C₉H₁₈N₂₀₂).

B. 2-Cyclopropyl-1-methyl-1H-imidazole: To a solution of N-(2,2-dimethoxyethyl)-N-methylcyclopropanecarboximidamide (40.0 g, 214 mmol, 1.00 eq) in MeOH (70.0 mL) and HCl (30.0 mL) under N₂. The mixture was stirred at 80° C. for 12 h under N₂. The mixture was poured into 50% aq NaOH and extracted with Ethyl acetate (2×300 mL). The combined organic layer was dried over Na₂SO₄ and concentrated under reduced pressure to get residue. The crude product was purified by column chromatography (SiO₂, Dichloromethane:Methanol=500:1 to 20:1, TLC:Dichloromethane:Methanol=20:1,R_{f}=0.30) to give the title compound (20.0 g, 163.mmol, 76.2% yield) as brown oil. ¹H NMR: (400 MHz, DMSO-d₆) δ 6.95-6.94 (m, 1H), 6.64 (s, 1H), 3.67-3.58 (m, 3H), 1.94-1.89 (m, 1H), 0.87-0.84 (m, 2H), 0.77-0.75 (m, 2H). (ESI⁺) m/z: 123.1 (M+H)⁺, (C₇H₁₀N₂).

C. 5-(2-Cyclopropyl-1-methyl-1H-imidazol-5-yl)-2-methylpyrimidine: To a solution of 2-cyclopropyl-1-methyl-1H-imidazole (1.00 g, 8.19 mmol, 1.00 eq) and 5-bromo-2-methylpyrimidine (4.25 g, 24.5 mmol, 3.00 eq) in DMF (10.0 mL) was added P(oxole)₃ (380 mg, 1.64 mmol, 0.20 eq), K₂CO₃ (2.26 g, 16.3 mmol, 2.00 eq) and Pd(OAc)₂ (367 mg, 1.64 mmol, 0.20 eq) under N₂. The mixture was stirred at 100° C. for 60 h under N₂. The reaction mixture was filtered through celite. The filtrate was concentrated to give residue. The residue was purified by preparative-HPLC (using a Phenomenex luna C18 (150×25 mm×10 μm) and gradient of 8.00%-38.0% acetonitrile in water containing 0.05% FA over 20 min at a flow rate of 25 mL/min) to give the title compound (550 mg, 2.36 mmol, 28.8% yield, 92.1% purity in LCMS at 220 nm) as light yellow solid. ¹H NMR: (400 MHz, DMSO-d₆) δ 8.80 (s, 2H), 7.01 (s, 1H), 3.67 (s, 3H), 2.65 (s, 3H), 2.07-1.98 (m, 1H), 0.95-0.92 (m, 2H), 0.85-0.84 (m, 2H). (ESI⁺) m/z: 215.0 (M+H)⁺, (C₁₂H₁₄N₄).

D. 5-(2-Cyclopropyl-1-methyl-1H-imidazol-5-yl)-2-methylpyrimidine: To a solution of 5-(2-cyclopropyl-3-methyl-imidazol-4-yl)-2-methyl-pyrimidine (300 mg, 1.40 mmol, 1.00 eq) in ACN (3.00 mL) was added NBS (249 mg, 1.40 mmol, 1.00 eq) at 0° C. The mixture was stirred at 0° C. for 1 h. The mixture was purified by preparative-TLC (Dichloromethane:Methanol=15:1,R_{f}=0.4) to give the title compound (340 mg, 1.16 mmol, 82.8% yield, 100% purity in LCMS at 220 nm) as light yellow solid. ¹H NMR: (400 MHz, DMSO-d₆) δ 8.79 (s, 2H), 3.59 (s, 3H), 2.68 (s, 3H), 2.09-2.05 (m, 1H), 0.99-0.95 (m, 2H), 0.87-0.85 (m, 2H). (ESI⁺) m/z: 293.0 (M+H)⁺, (C₁₂H₁₃BrN₄).

E. 3-(5-(2-cyclopropyl-1-methyl-5-(2-methylpyrimidin-5-yl)-1H-imidazol-4-yl)-1-oxoisoindolin-2-yl)piperidine-2,6-dione: To a solution of 5-(4-bromo-2-cyclopropyl-1-methyl-1H-imidazol-5-yl)-2-methylpyrimidine (150 mg, 511 μmol, 1.00 eq), 3-(1-oxo-5-(4,4,5,5-tetramethyl-1,3,2-dioxaborolan-2-yl)isoindolin-2-yl)piperidine-2,6-dione (757 mg, 2.05 mmol, 4.00 eq) and K₃PO₄ (325 mg, 1.53 mmol, 3.00 eq) in dioxane (5.00 mL) and H₂O (0.25 mL) was added Ru-Phos-Pd-G₃ (42.7 mg, 51.1 μmol, 0.10 eq) under N₂. The mixture was stirred at 100° C. for 4 h under N₂. The reaction mixture was concentrated under reduced pressure to get a residue at 45° C. The residue was purified by preparative-HPLC (using a Phenomenex luna C18 (150×25 mm×10 μm) and gradient of 0.00%-25.0% acetonitrile in water containing 0.05% TFA over 15 min at a flow rate of 25 mL/min) to give the title compound (210 mg, 438 μmol, 85.7% yield, 95.3% purity in HPLC at 220 nm) as white solid. ¹H NMR: (400 MHz, DMSO-d₆) δ 11.03-10.96 (m, 1H), 8.77-8.74 (m, 2H), 7.71-7.69 (m, 1H), 7.60 (s, 1H), 7.44-7.42 (m, 1H), 5.12-5.07 (m, 1H), 4.42-4.25 (m, 2H), 3.68-3.65 (m, 3H), 2.93-2.71 (m, 1H), 2.61-2.58 (m, 3H), 2.56-2.52 (m, 1H), 2.45-2.42 (m, 1H), 2.38-2.31 (m, 1H), 2.00-1.99 (m, 1H), 1.23-1.12 (m, 4H). (ESI⁺) m/z: 457.2 (M+H)⁺, (C₂₅H₂₄N₆O₃).

### Example 548

### Synthesis of 3-(5-(2-cyclopropyl-5-(2-cyclopropylpyrimidin-5-yl)-1-methyl-1H-imidazol-4-yl)-1-oxoisoindolin-2-yl)piperidine-2,6-dione

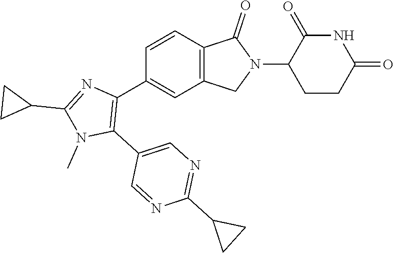 [SMILES: Cn1c(C2CC2)nc(-c2ccc3c(c2)CN(C2CCC(=O)NC2=O)C3=O)c1-c1cnc(C2CC2)nc1]

A. 2-Cyclopropyl-5-(2-cyclopropyl-1-methyl-1H-imidazol-5-yl)pyrimidine: To a solution of 2-cyclopropyl-1-methyl-1H-imidazole (600 mg, 4.92 mmol, 1.00 eq) and 5-bromo-2-cyclopropyl pyrimidine (1.47 g, 7.36 mmol, 1.50 eq) in DMF (6.00 mL) was added P(oxole)₃ (228 mg, 982 μmol, 0.20 eq), K₂CO₃ (1.36 g, 9.82 mmol, 2.00 eq) and Pd(OAc)₂ (220 mg, 9.82 μmol, 0.20 eq) under N₂. Then the mixture was stirred at 110° C. for 48 h. The mixture was concentrated under reduce pressure to get residue. The residue was purified by column chromatography (SiO₂, Dichloromethane:Methanol=500:1 to 50:1, TLC: Dichloromethane:Methanol=15:1, R_{f}=0.3) to give the title compound (400 mg, 1.53 mmol, 33.9% yield, 91.9% purity) in LCMS at 220 nm) as yellow solid. ¹H NMR: (400 MHz, DMSO-d₆) δ 8.73 (s, 2H), 6.98 (s, 1H), 3.66 (s, 3H), 2.25-2.22 (m, 1H), 2.05-2.02 (m, 1H), 1.08-1.04 (m, 4H), 0.94-0.85 (m, 4H). (ESI⁺) m/z: 241.0 (M+H)⁺. (C₁₄H₁₆N₄).

B. 5-(4-Bromo-2-cyclopropyl-1-methyl-1H-imidazol-5-yl)-2-cyclopropylpyrimidine: To a solution of 2-cyclopropyl-5-(2-cyclopropyl-1-methyl-1H-imidazol-5-yl) pyrimidine (400 mg, 1.66 mmol, 1.00 eq) in ACN (4.00 mL) was added NBS (354 mg, 1.99 mmol, 1.20 eq). Then the mixture was stirred at 25° C. for 2 h. Concentrated the reaction mixture under reduce pressure to get residue. The residue was purified by preparative-TLC (Dichloromethane:Methanol=15:1,R_{f}=0.4) to give the title compound (230 mg, 685 μmol, 43.4% yield, 95.1% purity in LCMS at 220 nm) as yellow solid. ¹H NMR: (400 MHz, DMSO-d₆) δ 8.72 (s, 2H), 3.58 (s, 3H), 2.29-2.25 (m, 1H), 2.09-2.05 (m, 1H), 1.12-1.07 (m, 2H), 1.06-1.04 (m, 2H), 0.96-0.85 (m, 2H), 0.83-0.77 (m, 2H). (ESI⁺) m/z: 320.9 (M+H)⁺. (C₁₄H₁₅BrN₄).

C. 3-(5-(2-cyclopropyl-5-(2-cyclopropylpyrimidin-5-yl)-1-methyl-1H-imidazol-4-yl)-1-oxoisoindolin-2-yl)piperidine-2,6-dione: To a solution of 5-(4-bromo-2-cyclopropyl-1-methyl-1H-imidazol-5-yl)-2-cyclopropylpyrimidine (150 mg, 469 μmol, 1.00 eq), 3-(1-oxo-5-(4,4,5,5-tetramethyl-1,3,2-dioxaborolan-2-yl)isoindolin-2-yl)piperidine-2,6-dione (695 mg, 1.88 mmol, 4.00 eq) and K₃PO₄ (299 mg, 1.41 mmol, 3.00 eq) in dioxane (6.00 mL) and H₂O (0.30 mL) was added Ru-Phos-Pd-G₃ (39.3 mg, 46.9 μmol, 0.10 eq) under N₂. The mixture was stirred at 100° C. for 4 h under N₂. The reaction mixture was concentrated under reduced pressure to get a residue at 45° C. The residue was purified by preparative-HPLC (using a Phenomenex luna C18 (150×25 mm×10 μm) and gradient of 4.00%-34.0% acetonitrile in water containing 0.50% TFA over 10 min at a flow rate of 25 mL/min) to give the title compound (76.6 mg, 156 μmol, 33.2% yield, 98.3% purity in HPLC at 220 nm) was obtained as white solid. ¹H NMR: (400 MHz, DMSO-d₆) δ 10.97-10.94 (m, 1H), 8.66-8.64 (m, 2H), 7.57-7.55 (m, 2H), 7.38-7.36 (m, 1H), 5.09-5.05 (m, 1H), 4.40-4.21 (m, 2H), 3.51 (s, 3H), 2.90-2.60 (m, 1H), 2.56-2.55 (m, 1H), 2.40-2.33 (m, 2H), 2.11-2.09 (m, 1H), 2.03-1.97 (m, 1H), 1.13-1.07 (m, 4H), 1.00-0.96 (m, 4H). (ESI⁺) m/z: 483.2 (M+H)⁺, (C₂₇H₂₆N₆O₃).

### Example 549

### Synthesis of 3-(5-(1-isopropyl-2-phenyl-1H-imidazol-4-yl)-1-oxoisoindolin-2-yl)piperidine-2,6-dione

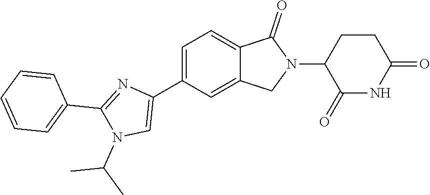 [SMILES: CC(C)n1cc(-c2ccc3c(c2)CN(C2CCC(=O)NC2=O)C3=O)nc1-c1ccccc1]

A. 2,4,5-Tribromo-1-isopropyl-1H-imidazole: To a solution of 2,4,5-tribromo-1H-imidazole (2.50 g, 8.20 mmol, 1.00 eq), K₂CO₃ (2.83 g, 20.5 mmol, 2.50 eq) in DMF (25.0 mL) was added 2-iodopropane (2.09 g, 12.3 mmol, 1.23 mL, 1.50 eq). The mixture was stirred at 60° C. for 12 h. The mixture was poured into H₂O (100 mL) and extracted with Ethyl acetate (3×70.0 mL). The combined organic layer was washed with saturated NaCl aqueous (3×60.0 mL), dried over Na₂SO₄, filtered to collect liquid and concentrated under reduced pressure to get the title compound (5.00 g, 14.3 mmol, 87.2% yield, 99.3% purity at 220 nm in LCMS) as yellow solid. ¹H NMR: (400 MHz, DMSO-d₆) δ 4.80-4.69 (m, 1H), 1.53-1.51 (d, J=8.0 Hz, 6H). (ESI⁺) m/z: 348.6 (M+H)⁺, (C₇H₁₀Br₂N₂).

B. 4,5-Dibromo-1-ethyl-2-phenyl-1H-imidazole: To a solution of 2,5-dibromo-1-isopropyl-4-methyl-1H-imidazole (1.00 g, 2.88 mmol, 1.00 eq), phenylboronic acid (369 mg, 3.03 mmol, 1.05 eq) and Pd(PPh₃)₄ (233 mg, 202 μmol, 0.07 eq) in Tol (10.0 mL) and MeOH (2.00 mL) was added Na₂CO₃ (2.00 M, 2.88 mL, 2.00 eq) under N₂. The mixture was stirred at 65° C. for 12 h under N₂. The mixture was poured into H₂O (80.0 mL) and extracted with Ethyl acetate (3×50.0 mL). The combined organic layer was washed with saturated NaCl aqueous (3×50.0 mL), dried over Na₂SO₄ and concentrated under reduced pressure to get residue. The residue was purified by column chromatography (SiO₂, Petroleum ether/Ethyl acetate=5/1, R_{f}=0.35) to give the title compound (1.00 g, 2.77 mmol, 33.5% yield, 95.2% purity in LCMS at 220 nm) as yellow solid. ¹H NMR: (400 MHz, DMSO-d₆) δ 7.54-7.45 (m, 5H), 4.64-4.53 (m, 1H), 1.51-1.49 (d, J=8.0 Hz, 6H). (ESI⁺) m/z: 343.0 (M+H)⁺, (C₁₂H₁₃BrN₂).

C. 3-(5-(1-Isopropyl-2-phenyl-1H-imidazol-4-yl)-1-oxoisoindolin-2-yl)piperidine-2,6-dione: To a solution of 4-bromo-1-isopropyl-2-phenyl-1H-imidazole (600 mg, 2.26 mmol, 1.00 eq), 3-(1-oxo-5-(4,4,5,5-tetramethyl-1,3,2-dioxaborolan-2-yl)isoindolin-2-yl)piperidine-2,6-dione (1.68 g, 4.53 mmol, 2.00 eq) and K₃PO₄ (960 mg, 4.53 mmol, 2.00 eq) in dioxane (10.0 mL) and H₂O (0.5 mL) was added Ru-Phos-Pd-G₃ (378 mg, 452 μmol, 0.20 eq) under N₂. The mixture was stirred at 100° C. for 2 h under N₂. The mixture was filtered to collect liquid and concentrated under reduced pressure to get residue. The crude product was purified by preparative-HPLC (using a Phenomenex luna C18 (150 mm×25 mm 10 μm) and gradient of 12-42% acetonitrile in water containing 0.05% FA over 15 min at a flow rate of 25 mL/min) to give the title compound (301 mg, 695 μmol, 31.0% yield, 98.9% purity in HPLC at 220 nm) as white solid. ¹H NMR: (400 MHz, DMSO-d₆) δ 11.0 (s, 1H), 8.54 (s, 1H), 8.09 (s, 1H), 8.03-8.01 (d, J=8.0 Hz, 1H), 7.85-7.80 (m, 1H), 7.66-7.65 (m, 5H), 5.16-5.12 (m, 1H), 4.56-4.51 (m, 2H), 4.43-4.38 (m, 1H), 2.96-2.89 (m, 1H), 2.64-2.59 (m, 1H), 2.46-2.41 (m, 1H), 2.05-2.01 (m, 1H), 1.51-1.49 (d, J=8.0 Hz, 6H). (ESI⁺) m/z: 428.9 (M+H)⁺, (C₂₅H₂₄N₄O₃).

### Example 550

### Synthesis of 3-(5-(1-isopropyl-5-phenyl-1H-pyrazol-3-yl)-1-oxoisoindolin-2-yl)piperidine-2,6-dione

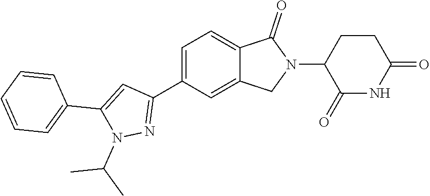 [SMILES: CC(C)n1nc(-c2ccc3c(c2)CN(C2CCC(=O)NC2=O)C3=O)cc1-c1ccccc1]

A. 3-Iodo-5-phenyl-1H-pyrazole: A mixture of 5-phenyl-1H-pyrazol-3-amine (10.0 g, 62.82-mmol, 1.00 eq), NaNO₂ (13.0 g, 188 mmol, 3.00 eq), TsOH (32.4 g, 188 mmol, 3.00 eq) in ACN (100 mL), H₂O (20.0 mL) was degassed and purged with N₂ for 3 times at 0° C. for 30 min, and then NaI (28.2 g, 188 mmol, 3.00 eq) was added and the mixture was stirred at 25° C. for 1 h under N₂ atmosphere. The reaction mixture was diluted with water 100 mL and extracted with EA 150 mL (3×50.0 mL). The combined organic layers were washed with brine 300 mL (2×150 mL), dried over Na₂SO₄, filtered and concentrated under reduced pressure to give a residue. The residue was purified by column chromatography (SiO₂, PE/EA=20/1 to 1/1, TLC: PE/EA=1/1, R_{f}=0.54). to give the title compound (4.74 g, 17.2 mmol, 27.4% yield, 98.1% purity in LCMS at 220 nm) as a black brown solid. ¹H NMR: (400 MHz, DMSO-d₆) δ 13.8-13.3 (m, 1H), 7.75-7.73 (m, 2H), 7.51-7.45 (m, 2H), 7.45-7.40 (m, 1H), 6.93 (s, 1H). (ESI⁺) m/z: 271.0 (M+H)⁺, (C₉H₇N₂I).

B. 3-Iodo-1-isopropyl-5-phenyl-1H-pyrazole: A mixture of 3-iodo-5-phenyl-1H-pyrazole (4.74 g, 17.2 mmol, 1.00 eq), 2-bromopropane (4.24 g, 34.4 mmol, 3.23 mL, 2.00 eq), Cs₂CO₃ (11.2 g, 34.4 mmol, 2.00 eq) in DMF (50.0 mL) was degassed and purged with N₂ for 3 times, and then the mixture was stirred at 25° C. for 10 h under N₂ atmosphere. The reaction mixture was extracted with EA 150 mL (3×50.0 mL). The combined organic layers were washed with brine 200 mL (2×100 mL), dried over Na₂SO₄, filtered and concentrated under reduced pressure to give a residue. The filtrate was purified by preparative-HPLC (using a Phenomenex luna C18 (250 mm×70 mm×15 μm) and gradient of 55%-80% acetonitrile in water containing 0.05% FA over 23 min at a flow rate of 140 mL/min) to get title compound (465 mg, 1.49 mmol, 8.65% yield, 100% purity in LCMS at 220 nm) was obtained as a yellow oil. ¹H NMR: (400 MHz, DMSO-d₆) δ 7.52-7.45 (m, 3H), 7.42-7.41 (m, 2H), 6.50 (s, 1H), 4.50-4.44 (m, 1H), 1.36-1.34 (m, 6H). (ESI⁺) m/z: 313.0 (M+H)⁺, (C₁₂H₁₃N₂I).

C. 3-(5-(1-Isopropyl-5-phenyl-1H-pyrazol-3-yl)-1-oxoisoindolin-2-yl)piperidine-2,6-dione: A mixture of 3-iodo-1-isopropyl-5-phenyl-pyrazole (465 mg, 1.49 mmol, 1.00 eq), 3-[1-oxo-5-(4,4,5,5-tetramethyl-1,3,2-dioxaborolan-2-yl)isoindolin-2-yl]piperidine-2,6-dione (1.65 g, 4.47 mmol, 3.00 eq), Ru-Phos-Pd-G₃ (124 mg, 148 μmol, 0.10 eq), K₃PO₄ (632 mg, 2.98 mmol, 2.00 eq) in dioxane (10.0 mL), H₂O (0.50 mL) was degassed and purged with N₂ for 3 times, and then the mixture was stirred at 100° C. for 1 h under N₂ atmosphere. The reaction mixture was filtered and concentrated under reduced pressure to give a residue. The filtrate was purified by preparative-HPLC (using a Phenomenex luna C18 (150 mm×25 mm×10 μm) and gradient of 41%-71% acetonitrile in water containing 0.05% FA over 15 min at a flow rate of 75 mL/min) to get title compound (130 mg, 297 μmol, 19.9% yield, 98.0% purity in HPLC at 220 nm) as a off-white solid. ¹H NMR: (400 MHz, DMSO-d₆) δ 11.0 (s, 1H), 8.09-8.08 (m, 1H), 8.02-8.00 (m, 1H), 7.77-7.75 (m, 1H), 7.57-7.50 (m, 5H), 6.90-6.98 (m, 1H), 5.15-5.20 (m, 1H), 4.59-4.37 (m, 3H), 2.97-2.86 (m, 1H), 2.63-2.57 (m, 1H), 2.44-2.36 (m, 1H), 2.04-2.01 (m, 1H), 1.46-1.44 (m, 6H). (ESI⁺) m/z: 429.0 (M+H)⁺, (C₂₅H₂₄O₃N₄).

### Example 551

### Synthesis of 3-(1-oxo-5-(3-phenyl-1H-pyrazol-1-yl)isoindolin-2-yl)piperidine-2,6-dione

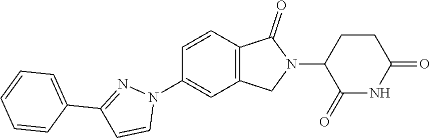 [SMILES: O=C1CCC(N2Cc3cc(-n4ccc(-c5ccccc5)n4)ccc3C2=O)C(=O)N1]

A. 5-Amino-5-oxo-4-(1-oxo-5-(3-phenyl-1H-pyrazol-1-yl)isoindolin-2-yl)pentanoic acid (Batches in parallel 6): To a solution of tert-butyl 5-amino-4-(5-bromo-1-oxo-isoindolin-2-yl)-5-oxo-pentanoate (0.10 g, 251 μmol, 1.00 eq), 3-phenyl-1H-pyrazole (29.0 mg, 201 μmol, 0.80 eq) in DMF (2.00 mL) and H₂O (0.20 mL) was added Cs₂CO₃ (328 mg, 1.01 mmol, 4.00 eq), CuI (1.44 mg, 7.55 μmol, 0.03 eq) and 8-hydroxyquinolin (1.10 mg, 7.55 μmol, 0.03 eq). The reaction mixture was stirred at 150° C. for 0.5 h in microwave. The reaction mixture was filtered and concentrated in vacuum to give residue. The residue was purified by preparative-HPLC (using a Welch Xtimate C18 (200 mm×40 mm 10 μm) and gradient of 14-44% acetonitrile in water containing 0.05% TFA over 12 min at a flow rate of 25.0 mL/min) to give the title compound (40.0 mg, 98.9 μmol, 6.55% yield) as white solid. ¹H NMR: (400 MHz, DMSO-d₆) δ 13.0 (s, 1H), 8.69 (d, J=2.8 Hz, 1H), 8.19 (s, 1H), 8.09-8.06 (m, 1H), 7.97-7.95 (m, 2H), 7.83 (d, J=8.4 Hz, 1H), 7.49-7.45 (m, 2H), 7.40-7.36 (m, 1H), 7.25 (s, 1H), 7.12 (d, J=2.8 Hz, 1H), 6.75 (s, 1H), 4.78-4.76 (m, 1H), 4.57 (s, 2H), 2.35-2.25 (m, 1H), 2.12-2.05 (m, 3H). (ESI⁺) m/z: 405 (M+H)⁺, (C₂₂H₂₀N₄O₄).

B. 3-(1-Oxo-5-(3-phenyl-1H-pyrazol-1-yl)isoindolin-2-yl)piperidine-2,6-dione: A solution of 5-amino-5-oxo-4-[1-oxo-5-(5-phenylpyrazol-1-yl)isoindolin-2-yl]pentanoic acid (30.0 mg, 74.1 μmol, 1.00 eq) and CDI (96.2 mg, 593 μmol, 8.00 eq) in ACN (1.00 mL) was stirred at 80° C. for 12 h. The reaction mixture was concentrated in vacuum to give residue. The residue was triturated with ACN (2.00 mL) at 0° C. for 30 min to give the title compound (24.1 mg, 62.2 μmol, 83.9% yield, 99.7% purity in HPLC at 220 nm) as white solid. ¹H NMR: (400 MHz, DMSO-d₆) δ 10.9 (s, 1H), 8.71 (d, J=2.8 Hz, 1H), 8.19 (s, 1H), 8.11-8.09 (s, 1H), 7.98-7.96 (m, 2H), 7.86 (d, J=8.0 Hz, 1H), 7.49-7.46 (m, 2H), 7.40-7.37 (m, 1H), 7.12 (d, J=2.8 Hz, 1H), 5.16-5.12 (m, 1H), 4.58-4.41 (m, 2H), 2.97-2.90 (m, 1H), 2.61-2.56 (m, 1H), 2.46-2.43 (m, 1H), 2.06-2.04 (m, 1H). (ESI⁺) m/z: 387.1 (M+H)⁺, (C₂₂H₁₈N₄O₃).

### Example 552

### Synthesis of 3-(1-oxo-5-(4-phenyl-1H-imidazol-1-yl)isoindolin-2-yl)piperidine-2,6-dione

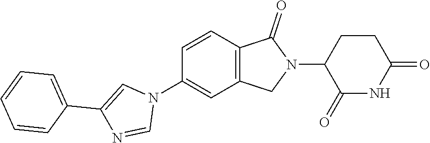 [SMILES: O=C1CCC(N2Cc3cc(-n4cnc(-c5ccccc5)c4)ccc3C2=O)C(=O)N1]

A. 5-Amino-5-oxo-4-(1-oxo-5-(4-phenyl-1H-imidazol-1-yl)isoindolin-2-yl)pentanoic acid: To a solution of tert-butyl 5-amino-4-(5-bromo-1-oxo-isoindolin-2-yl)-5-oxo-pentanoate (500 mg, 1.26 mmol, 1.00 eq) in DMF (10.0 mL) and H₂O (1.00 mL) was added 4-phenyl-1H-imidazole (181 mg, 1.26 mmol, 1.00 eq), Cs₂CO₃ (1.64 g, 5.03 mmol, 4.00 eq), CuI (7.19 mg, 37.7 μmol, 0.03 eq) and quinolin-8-ol (5.48 mg, 37.7 μmol, 6.53 μL, 0.03 eq) under N₂. The mixture was stirred in a microwave reactor at 145° C. for 0.5 h under N₂. After the reaction was completed, the reaction mixture was filtered and the filtrate was concentrated in vacuum to get a residue. The residue was purified by preparative-HPLC (using a Welch Xtimate C18 (200 mm×40 mm×10 μm) and gradient of 1-30% acetonitrile in water containing 0.5% TFA over 12 min at a flow rate of 25.0 mL/min) to give the title compound (200 mg, 425 μmol, 33.8% yield, 86.1% purity in HPLC at 220 nm) as a yellow solid. ¹H NMR: (400 MHz, DMSO-d₆) δ 8.86 (s, 1H), 8.54 (d, J=0.8 Hz, 1H), 8.05 (s, 1H), 7.94-7.87 (m, 4H), 7.46 (t, J=8.0 Hz, 2H), 7.33 (t, J=7.2 Hz, 1H), 7.26 (s, 1H), 6.76 (s, 1H), 4.80-4.76 (m, 1H), 4.59 (s, 2H), 2.36-2.29 (m, 1H), 2.34-2.29 (m, 1H), 2.14-2.07 (m, 3H). (ESI⁺) m/z: 405.1 (M+H)⁺, (C₂₂H₂₀N₄O₄).

B. 3-(1-Oxo-5-(4-phenyl-1H-imidazol-1-yl)isoindolin-2-yl)piperidine-2,6-dione: To a solution of 5-amino-5-oxo-4-[1-oxo-5-(4-phenylimidazol-1-yl)isoindolin-2-yl]pentanoic acid (120 mg, 255 μmol, 1.00 eq) in ACN (1.00 mL) was added CDI (124 mg, 766 μmol, 3.00 eq). The mixture was stirred at 100° C. for 2 h under N₂. After the reaction was completed, the reaction mixture was filtered and the filtrate was concentrated in vacuum to give a residue. The residue was purified by preparative-HPLC (using a Welch Xtimate C18 (150 mm×25 mm×5 μm) and gradient of 10-40% acetonitrile in water containing 0.5% TFA over 2 min at a flow rate of 25.0 mL/min) to give the title compound (82.2 mg, 211 μmol, 82.6% yield, 99.1% purity in HPLC at 220 nm) as an off-white solid. ¹H NMR: (400 MHz, DMSO-d₆) δ 11.0 (s, 1H), 8.89 (s, 1H), 8.56 (s, 1H), 8.06 (s, 1H), 7.94 (s, 2H), 7.88 (d, J=7.2 Hz, 2H), 7.46 (t, J=7.6 Hz, 2H), 7.33 (t, J=7.2 Hz, 1H), 5.15 (dd, J=13.2, 5.2 Hz, 1H), 4.50 (dd, J=49.6, 17.6 Hz, 2H), 2.99-2.92 (m, 1H), 2.65-2.59 (m, 1H), 2.48-2.44 (m, 1H), 2.08-2.04 (m, 1H). (ESI⁺) m/z: 387.1 (M+H)⁺, (C₂₂H₁₈N₄O₃).

### Example 553

### Synthesis of 3-(5-(1-isopropyl-5-phenyl-1H-1,2,4-triazol-3-yl)-1-oxoisoindolin-2-yl)piperidine-2,6-dione

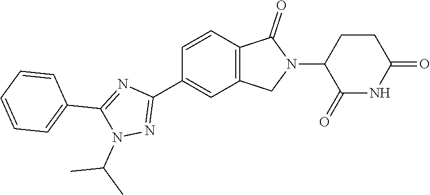 [SMILES: CC(C)n1nc(-c2ccc3c(c2)CN(C2CCC(=O)NC2=O)C3=O)nc1-c1ccccc1]

A. 4-Bromo-1-isopropyl-5-phenyl-1H-pyrazole: To a solution of 3,5-dibromo-1H-1,2,4-triazole (2.00 g, 8.82 mmol, 1.00 eq) in DMF (20.0 mL) was added NaH (528 mg, 13.2 mmol, 60.0% purity, 1.50 eq) at 0° C. under N₂. The mixture was stirred at 25° C. under N₂ for 0.5 h. Then the mixture was added 2-iodopropane (3.00 g, 17.6 mmol, 1.76 mL, 2.00 eq) at 25° C. under N₂. The mixture was stirred at 25° C. for 2.5 h under N₂. After the reaction was completed, the reaction mixture was poured into saturated NH₄Cl aqueous solution (30.0 mL) and extracted with ethyl acetate (3×30.0 mL). The combined organic layer was washed with brine (30 mL), dried over Na₂SO₄, filtered and the filtrate was concentrated in vacuum to get a residue. The residue was purified by column chromatography (SiO₂, Petroleum ether: Ethyl acetate=100:1 to 3:1,R_{f}=0.70 (Petroleum ether:Ethyl acetate=3:1)) to give the title compound (900 mg, 3.33 mmol, 37.8% yield, 99.6% purity in LCMS at 220 nm) as white oil. (ESI⁺) m/z: 267.9 (M+H)⁺, (C₅H₇Br₂N₃).

B. 3-Bromo-1-isopropyl-5-phenyl-1H-1,2,4-triazole: To a solution of 3,5-dibromo-1-isopropyl-1,2,4-triazole (800 mg, 2.96 mmol, 1.00 eq) in MeCN (12.0 mL) and H₂O (4.00 mL) was added phenylboronic acid (289 mg, 2.37 mmol, 0.80 eq), K₂CO₃ (1.02 g, 7.41 mmol, 2.50 eq) and Pd(dppf)Cl₂ (216 mg, 296 μmol, 0.10 eq) under N₂. The mixture was stirred at 25° C. for 2 h under N₂. After the reaction was completed, the reaction mixture was filtered and the filtrate was concentrated in vacuum to get a residue. The residue was purified by preparative-TLC (Petroleum ether:Ethyl acetate=5:1, R_{f}=0.50) to give the title compound (50.0 mg, 153 μmol, 5.17% yield, 81.5% purity in LCMS at 220 nm) as a white solid. ¹H NMR: (400 MHz, DMSO-d₆) δ 7.65-7.62 (m, 2H), 7.60-7.57 (m, 3H), 4.70-4.62 (m, 1H), 1.42 (d, J=6.4 Hz, 6H). (ESI⁺) m/z: 266.0 (M+H)⁺, (C₁₁H₁₂BrN₃).

C. 3-(5-(1-Isopropyl-5-phenyl-1H-1,2,4-triazol-3-yl)-1-oxoisoindolin-2-yl)piperidine-2,6-dione: To a solution of 3-bromo-1-isopropyl-5-phenyl-1,2,4-triazole (50.0 mg, 153 μmol, 1.00 eq) in dioxane (1.00 mL) and H₂O (0.05 mL) was added 3-[1-oxo-5-(4,4,5,5-tetramethyl-1,3,2-dioxaborolan-2-yl)isoindolin-2-yl]piperidine-2,6-dione (141 mg, 382 μmol, 2.50 eq), K₃PO₄ (65.0 mg, 306 μmol, 2.00 eq) and Ru-Phos-Pd-G₃ (12.8 mg, 15.3 μmol, 0.10 eq) under N₂. The mixture was stirred at 100° C. for 2 h under N₂. After the reaction was completed, the reaction mixture was filtered and the filtrate was concentrated in vacuum to give a residue. The residue was purified by preparative-TLC (Dichloromethane:Methanol=10:1, R_{f}=0.50) and by preparative-HPLC (using a Phenomenex Luna C18 (150 mm×25 mm×10 μm) and gradient of 29-59% acetonitrile in water containing 0.5% TFA over 14 min at a flow rate of 25.0 mL/min) to give the title compound (17.0 mg, 39.4 μmol, 25.9% yield, 99.6% purity in HPLC at 220 nm) as a white solid. ¹H NMR: (400 MHz, MeOD) δ 8.31 (t, J=8.0 Hz, 2H), 7.91 (d, J=8.0 Hz, 1H), 7.72-7.69 (m, 2H), 7.65-7.62 (m, 3H), 5.20 (dd, J=13.2, 4.8 Hz, 1H), 4.81-4.75 (m, 1H), 4.60 (dd, J=25.6, 17.2 Hz, 2H), 2.95-2.89 (m, 1H), 2.84-2.82 (m, 1H), 2.57-2.53 (m, 1H), 2.25-2.22 (m, 1H). 1.58 (d, J=6.4 Hz, 6H). (ESI⁺) m/z: 430.1 (M+H)⁺, (C₂₄H₂₃N₅O₃).

### Example 554

### Synthesis of 3-(5-(1-methyl-5-(piperidin-1-yl)-1H-1,2,4-triazol-3-yl)-1-oxoisoindolin-2-yl) piperidine-2,6-dione

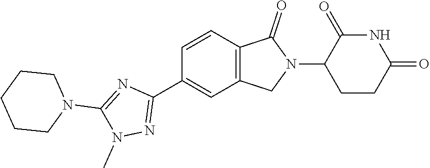 [SMILES: Cn1nc(-c2ccc3c(c2)CN(C2CCC(=O)NC2=O)C3=O)nc1N1CCCCC1]

A. 1-(3-Bromo-1-methyl-1H-1,2,4-triazol-5-yl)piperidine: To a solution of 3,5-dibromo-1-methyl-1,2,4-triazole (500 mg, 2.08 mmol, 1.00 eq) in NMP (10.0 mL) was added K₂CO₃ (573 mg, 4.15 mmol, 2.00 eq) and piperidine (176 mg, 2.08 mmol, 204 μL, 1.00 eq). The mixture was stirred in a microwave reactor at 140° C. for 0.75 h under N₂. After the reaction was completed, the reaction mixture was filtered and the filtrate was extracted with ethyl acetate (3×30.0 mL) and the combined organic layer was washed with brine (30 mL), dried over Na₂SO₄, concentrated in vacuum to get a residue. The residue was purified by preparative-TLC (Petroleum ether:Ethyl acetate=3:1, R_{f}=0.50) to give the title compound (370 mg, 1.44 mmol, 72.7% yield, 95.1% purity in LCMS at 220 nm) as yellow oil. ¹H NMR: (400 MHz, DMSO-d₆) δ 3.61 (s, 3H), 3.08 (t, J=5.6 Hz, 4H), 1.64-1.54 (m, 6H). (ESI⁺) m/z: 245.0 (M+H)⁺, (C₈H₁₃BrN₄).

B. 3-(5-(1-Methyl-5-(piperidin-1-yl)-1H-1,2,4-triazol-3-yl)-1-oxoisoindolin-2-yl)piperidine-2,6-dione: To a solution of 1-(5-bromo-2-methyl-1,2,4-triazol-3-yl)piperidine (350 mg, 1.36 mmol, 1.00 eq) in dioxane (4.00 mL) and H₂O (0.20 mL) was added 3-[1-oxo-5-(4,4,5,5-tetramethyl-1,3,2-dioxaborolan-2-yl)isoindolin-2-yl]piperidine-2,6-dione (1.26 g, 3.39 mmol, 2.50 eq), Ru-Phos-Pd-G₃ (113 mg, 135 μmol, 0.10 eq) and K₃PO₄ (576 mg, 2.72 mmol, 2.00 eq) under N₂. The mixture was stirred at 100° C. for 2 h under N₂. After the reaction was completed, the reaction mixture was filtered and the filtrate was concentrated in vacuum to give a residue. The residue was purified by preparative-TLC (Dichloromethane:Methanol=10:1, R_{f}=0.50) and by preparative-HPLC (using a Phenomenex Luna C18 (200 mm×40 mm×10 μm) and gradient of 10-40% acetonitrile in water containing 0.5% TFA over 14 min at a flow rate of 25.0 mL/min) to give the title compound (152 mg, 370 μmol, 27.5% yield, 99.2% purity in HPLC at 220 nm) as an off-white solid. ¹H NMR: (400 MHz, DMSO-d₆) δ 11.0 (s, 1H), 8.12 (s, 1H), 8.06 (d, J=8.0 Hz, 1H), 7.76 (d, J=8.0 Hz, 1H), 5.12 (dd, J=13.2, 5.2 Hz, 1H), 4.44 (dd, J=49.2, 17.2 Hz, 2H), 3.72 (s, 3H), 3.16 (t, J=5.6 Hz, 4H), 2.96-2.87 (m, 1H), 2.60 (d, J=17.2 Hz, 1H), 2.42-2.37 (m, 1H), 2.04-1.99 (m, 1H), 1.70-1.58 (m, 6H). (ESI⁺) m/z: 409.1 (M+H)⁺, (C₂₁H₂₄N₆O₃).

### Examples 555-587

The compounds of Example 555-587 were prepared according to the procedure shown in Scheme 5:

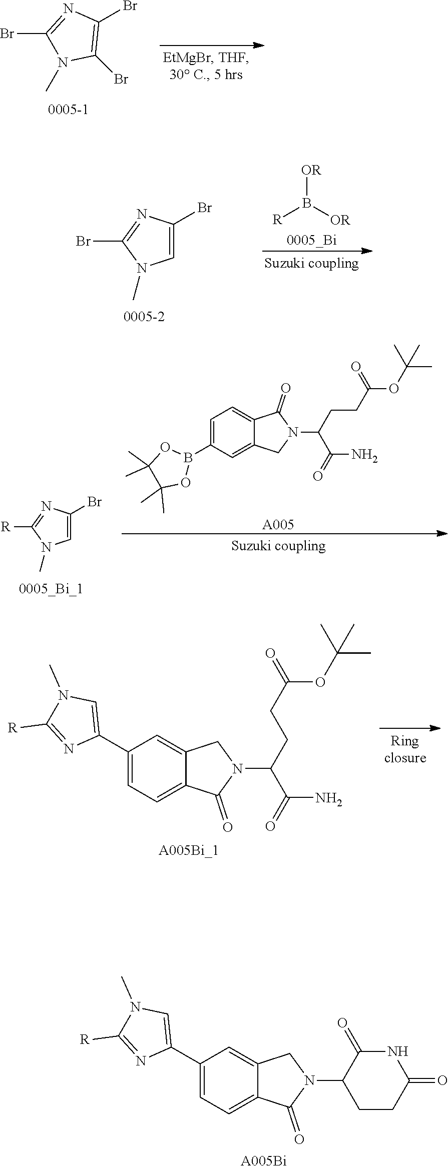

Experimental Details for Key Intermediate Synthesis

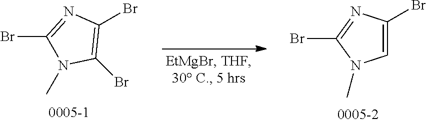

A mixture of 0005-1 (15.0 g, 47.0 mmol, 1.00 eq), EtMgBr (3.00 M, 17.3 mL, 1.10 eq) in THF (200 mL) was degassed and purged with N₂ for 3.00 times, and then the mixture was stirred at 30° C. for 5 hrs under N₂ atmosphere. LCMS (EC16219-6-P1F1) showed of desired 0005-2 was detected. The reaction mixture was quenched by addition NH₄Cl 200 mL at 0° C., and then extracted with EA 600 mL (200 mL×3). The combined organic layers were washed with H₂O 300 mL (100 mL×3), dried over Na₂SO₄, filtered and concentrated under reduced pressure to give a residue. The residue was purified by flash silica gel chromatography (ISCO®; 80 g SepaFlash® Silica Flash Column, Eluent of 0˜ 30% Ethyl acetate/Petroleum ether gradient @ 100 mL/min). 0005-2 (3.50 g, 14.6 mmol, 31.0% yield, 100% purity) was obtained as a white solid. Indicated by ¹H NMR. LCMS: m/z=240.8 [M+H]+; ¹H NMR: (chloroform-d, 400 MHz) δ=6.88 (s, 1H), 3.54 (s, 3H).

Step 1 Suzuki Coupling

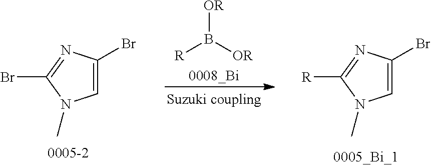

Condition 1

To a vial containing a solution of 0005-2 (0.20 mmol, 1.00 eq) and 0005_Bi (0.20 mmol, 1.00 eq) in Dioxane (1.50 mL) was added K₃PO₄ (1.50 M in H₂O, 0.60 mmol, 3.00 eq), Tri(4-fluorophenyl)phosphine (0.02 mmol, 0.10 eq) and Pd(OAc)₂ (0.02 μmol, 0.10 eq) under protection of N₂. The mixture was stirred at 80° C. for 16 hrs. Spot checked by LCMS. The reaction mixture was diluted with H₂O (2.00 mL) and extracted with ethyl acetate (2.00 mL×3). The combined organic layers were washed with brine (2.00 mL), dried over Na₂SO₄, filtered and concentrated under reduced pressure to give crude intermediates used for next step.

Condition 2

To a vial containing a solution of 0005-2 (0.20 mmol, 1.00 eq) and 0005_Bi (0.2 mmol, 1.00 eq) in Dioxane (1.50 mL) was added K₃PO₄ (1.50 M in H₂O, 0.60 mmol, 3.00 eq), and Pd(PPh₃)₄ (0.020 mmol, 0.10 eq) under protection of N₂. The mixture was stirred at 40° C. for 1 hr. The resulting mixture was stirred at 80° C. for 1 hr. Spot checked by LCMS. The reaction mixture was diluted with H₂O (2.00 mL) and extracted with ethyl acetate (2.00 mL×3). The combined organic layers were washed with brine (2.00 mL), dried over Na₂SO₄, filtered and concentrated under reduced pressure to give crude intermediates used for next step.

Condition 3

To a vial containing a solution of 0005-2 (0.20 mmol, 1.00 eq) and 0005_Bi (0.20 mmol, 1.00 eq) in Dioxane (1.50 mL) was added Na₂CO₃ (2.00 M in H₂O, 0.60 mmol, 5.00 eq), and Pd(PPh₃)₂Cl₂ (0.02 mmol, 0.10 eq) under protection of N₂. The mixture was stirred at 40° C. for 1 hr. The resulting mixture was stirred at 80° C. for 2 hrs. Spot checked by LCMS. The reaction mixture was diluted with H₂O (2.00 mL) and extracted with ethyl acetate (2.00 mL×3). The combined organic layers were washed with brine (2.00 mL), dried over Na₂SO₄, filtered and concentrated under reduced pressure to give crude intermediates used for next step.

Condition 4

To a vial containing a solution of 0005-2 (0.20 mmol, 1.00 eq) and 0005_Bi (0.20 mmol, 1.00 eq) in Dioxane (1.50 mL) was added Na₂CO₃ (1.50 M in H₂O, 0.60 mmol, 3.00 eq), and Pd(PPh₃)₂Cl₂ (0.02 mmol, 0.10 eq) under protection of N₂. The mixture was stirred at 80° C. for 16 hrs. Spot checked by LCMS. The reaction mixture was diluted with H₂O (2.00 mL) and extracted with ethyl acetate (2.00 mL×3). The combined organic layers were washed with brine (2.00 mL), dried over Na₂SO₄, filtered and concentrated under reduced pressure to give crude intermediates used for next step.

Step 2 Suzuki Coupling

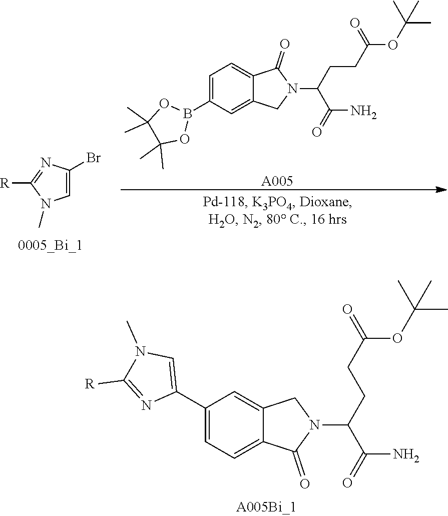

To a vial containing a solution of A005 (0.15 mmol, 1.00 eq) and 0005_Bi_1 (˜0.15 mmol, 1.00 eq) in Dioxane (1.00 mL) was added K₃PO₄ (1.50 M in H₂O, 0.45 mmol, 3.00 eq), and Pd-118 (0.015 mmol, 0.10 eq) under protection of N₂. The mixture was stirred at 80° C. for 16 hrs. Spot checked by LCMS. The reaction mixture was diluted with H₂O (2.00 mL) and extracted with ethyl acetate (2.00 mL×3). The combined organic layers were washed with brine (2.00 mL), dried over Na₂SO₄, filtered and concentrated under reduced pressure to give crude intermediates used for next step.

Step 3 Ring Closure

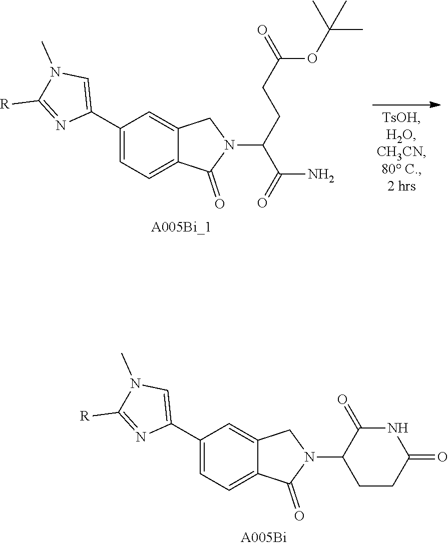

To a vial containing a solution of A005Bi_1 (0.10 mmol, 1.00 eq) in CH₃CN (1.00 mL) was added TsOH·H₂O (0.80 mmol, 8.00 eq). The mixture was stirred at 80° C. for 2 hrs.

Spot checked by LCMS. The residue was concentrated under reduced pressure and purified by prep-HPLC to give final product.

| | | MW + 1 | Step 1 |
|---|---|---|---|
| Example | Structure | (Observed) | Conditions |
| 555 | 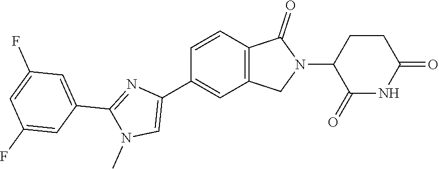 [SMILES: Cn1cc(-c2ccc3c(c2)CN(C2CCC(=O)NC2=O)C3=O)nc1-c1cc(F)cc(F)c1] | 437.2 | 2 |
| 556 | 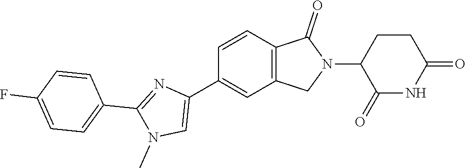 [SMILES: Cn1cc(-c2ccc3c(c2)CN(C2CCC(=O)NC2=O)C3=O)nc1-c1ccc(F)cc1] | 419.2 | 2 |
| 557 | 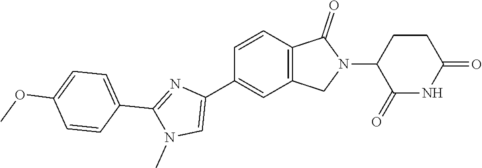 [SMILES: COc1ccc(-c2nc(-c3ccc4c(c3)CN(C3CCC(=O)NC3=O)C4=O)cn2C)cc1] | 431.3 | 1 |
| 558 | 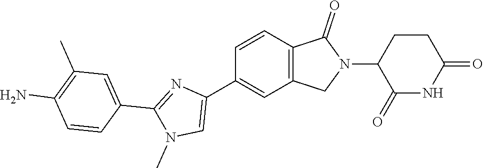 [SMILES: Cc1cc(-c2nc(-c3ccc4c(c3)CN(C3CCC(=O)NC3=O)C4=O)cn2C)ccc1N] | 430.3 | 2 |
| 559 | 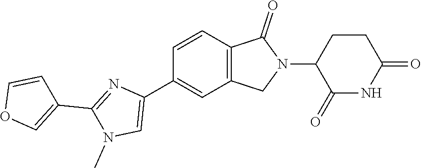 [SMILES: Cn1cc(-c2ccc3c(c2)CN(C2CCC(=O)NC2=O)C3=O)nc1-c1ccoc1] | 391.2 | 2 |
| 560 | 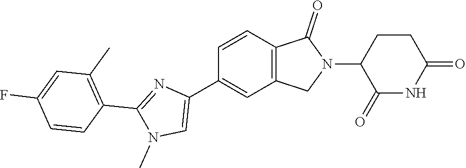 [SMILES: Cc1cc(F)ccc1-c1nc(-c2ccc3c(c2)CN(C2CCC(=O)NC2=O)C3=O)cn1C] | 433.3 | 2 |
| 561 | 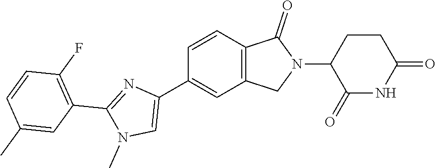 [SMILES: Cc1ccc(F)c(-c2nc(-c3ccc4c(c3)CN(C3CCC(=O)NC3=O)C4=O)cn2C)c1] | 433.3 | 2 |
| 562 | 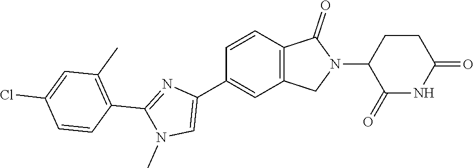 [SMILES: Cc1cc(Cl)ccc1-c1nc(-c2ccc3c(c2)CN(C2CCC(=O)NC2=O)C3=O)cn1C] | 449.3 | 2 |
| 563 | 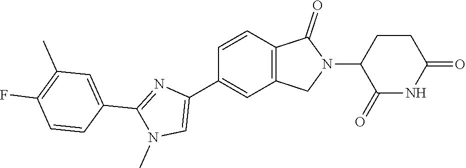 [SMILES: Cc1cc(-c2nc(-c3ccc4c(c3)CN(C3CCC(=O)NC3=O)C4=O)cn2C)ccc1F] | 433.3 | 2 |
| 564 | 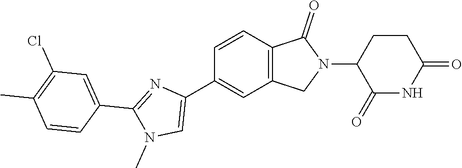 [SMILES: Cc1ccc(-c2nc(-c3ccc4c(c3)CN(C3CCC(=O)NC3=O)C4=O)cn2C)cc1Cl] | 449.2 | 2 |
| 565 | 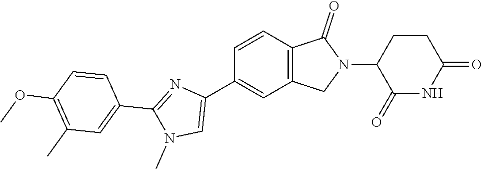 [SMILES: COc1ccc(-c2nc(-c3ccc4c(c3)CN(C3CCC(=O)NC3=O)C4=O)cn2C)cc1C] | 445.3 | 1 |
| 566 | 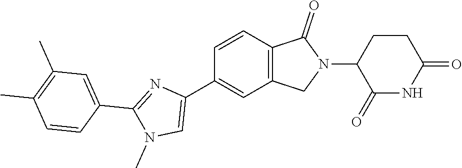 [SMILES: Cc1ccc(-c2nc(-c3ccc4c(c3)CN(C3CCC(=O)NC3=O)C4=O)cn2C)cc1C] | 429.3 | 1 |
| 567 | 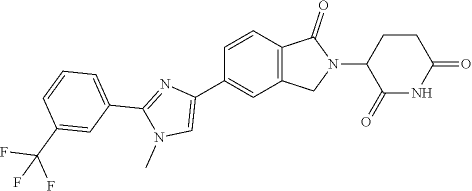 [SMILES: Cn1cc(-c2ccc3c(c2)CN(C2CCC(=O)NC2=O)C3=O)nc1-c1cccc(C(F)(F)F)c1] | 459.2 | 1 |
| 568 | 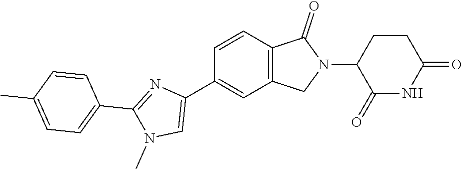 [SMILES: Cc1ccc(-c2nc(-c3ccc4c(c3)CN(C3CCC(=O)NC3=O)C4=O)cn2C)cc1] | 415.3 | 1 |
| 569 | 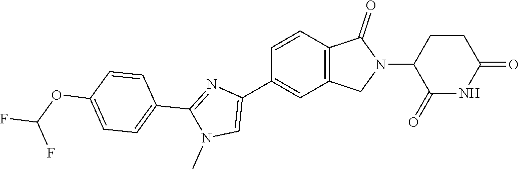 [SMILES: Cn1cc(-c2ccc3c(c2)CN(C2CCC(=O)NC2=O)C3=O)nc1-c1ccc(OC(F)F)cc1] | 467.2 | 1 |
| 570 | 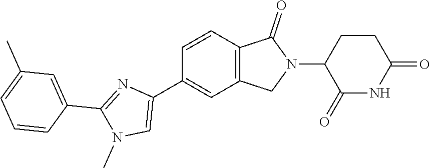 [SMILES: Cc1cccc(-c2nc(-c3ccc4c(c3)CN(C3CCC(=O)NC3=O)C4=O)cn2C)c1] | 415.3 | 1 |
| 571 | 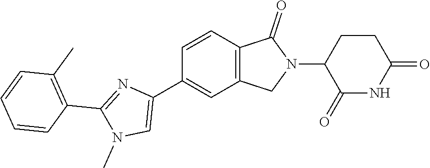 [SMILES: Cc1ccccc1-c1nc(-c2ccc3c(c2)CN(C2CCC(=O)NC2=O)C3=O)cn1C] | 415.3 | 2 |
| 572 | 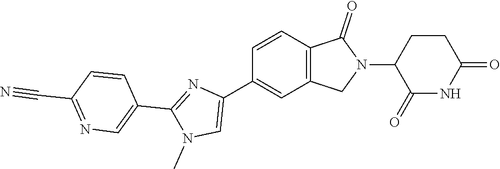 [SMILES: Cn1cc(-c2ccc3c(c2)CN(C2CCC(=O)NC2=O)C3=O)nc1-c1ccc(C#N)nc1] | 427.2 | 2 |
| 573 | 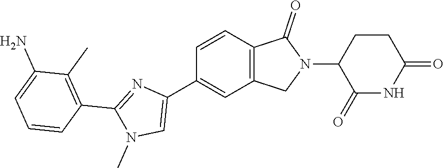 [SMILES: Cc1c(N)cccc1-c1nc(-c2ccc3c(c2)CN(C2CCC(=O)NC2=O)C3=O)cn1C] | 430.3 | 2 |
| 574 | 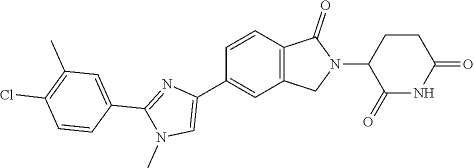 [SMILES: Cc1cc(-c2nc(-c3ccc4c(c3)CN(C3CCC(=O)NC3=O)C4=O)cn2C)ccc1Cl] | 449.2 | 1 |
| 575 | 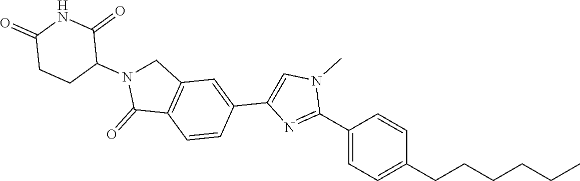 [SMILES: CCCCCCc1ccc(-c2nc(-c3ccc4c(c3)CN(C3CCC(=O)NC3=O)C4=O)cn2C)cc1] | 485.4 | 1 |
| 576 | 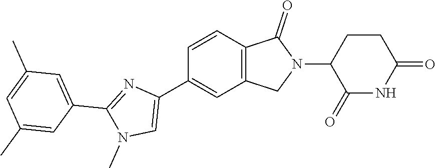 [SMILES: Cc1cc(C)cc(-c2nc(-c3ccc4c(c3)CN(C3CCC(=O)NC3=O)C4=O)cn2C)c1] | 429.3 | 1 |
| 577 | 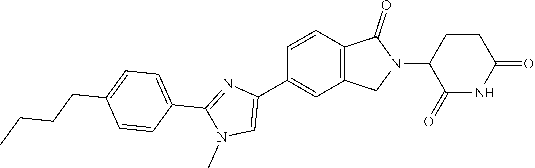 [SMILES: CCCCc1ccc(-c2nc(-c3ccc4c(c3)CN(C3CCC(=O)NC3=O)C4=O)cn2C)cc1] | 457.3 | 1 |
| 578 | 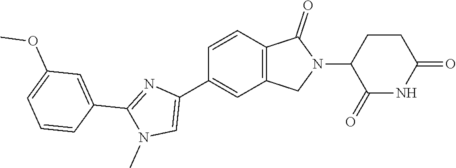 [SMILES: COc1cccc(-c2nc(-c3ccc4c(c3)CN(C3CCC(=O)NC3=O)C4=O)cn2C)c1] | 431.3 | 1 |
| 579 | 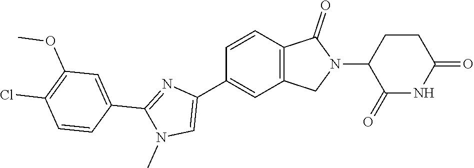 [SMILES: COc1cc(-c2nc(-c3ccc4c(c3)CN(C3CCC(=O)NC3=O)C4=O)cn2C)ccc1Cl] | 465.2 | 2 |
| 580 | 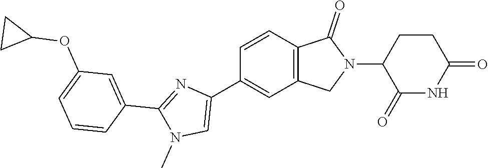 [SMILES: Cn1cc(-c2ccc3c(c2)CN(C2CCC(=O)NC2=O)C3=O)nc1-c1cccc(OC2CC2)c1] | 457.3 | 2 |
| 581 | 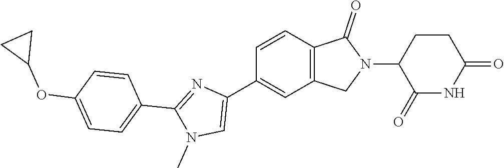 [SMILES: Cn1cc(-c2ccc3c(c2)CN(C2CCC(=O)NC2=O)C3=O)nc1-c1ccc(OC2CC2)cc1] | 457.3 | 2 |
| 582 | 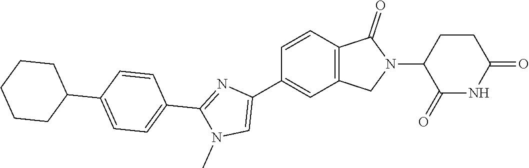 [SMILES: Cn1cc(-c2ccc3c(c2)CN(C2CCC(=O)NC2=O)C3=O)nc1-c1ccc(C2CCCCC2)cc1] | 483.4 | 1 |
| 583 | 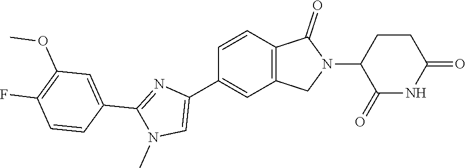 [SMILES: COc1cc(-c2nc(-c3ccc4c(c3)CN(C3CCC(=O)NC3=O)C4=O)cn2C)ccc1F] | 449.3 | 2 |
| 584 | 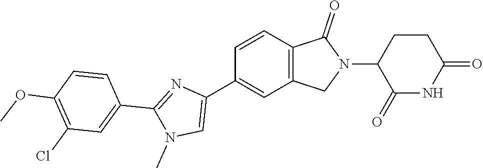 [SMILES: COc1ccc(-c2nc(-c3ccc4c(c3)CN(C3CCC(=O)NC3=O)C4=O)cn2C)cc1Cl] | 465.2 | 2 |
| 585 | 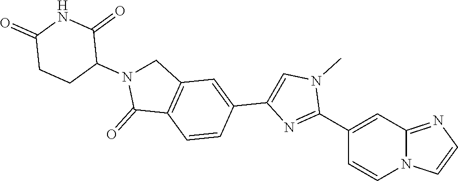 [SMILES: Cn1cc(-c2ccc3c(c2)CN(C2CCC(=O)NC2=O)C3=O)nc1-c1ccn2ccnc2c1] | 441.3 | 2 |
| 586 | 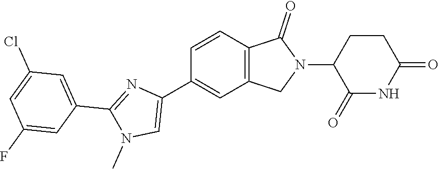 [SMILES: Cn1cc(-c2ccc3c(c2)CN(C2CCC(=O)NC2=O)C3=O)nc1-c1cc(F)cc(Cl)c1] | 453.2 | 2 |
| 587 | 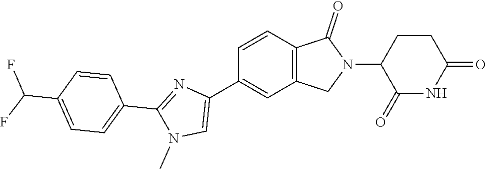 [SMILES: Cn1cc(-c2ccc3c(c2)CN(C2CCC(=O)NC2=O)C3=O)nc1-c1ccc(C(F)F)cc1] | 451.2 | 2 |

### Examples 588-593

The compounds of Examples 588-593 were prepared according to the procedure in Scheme 6:

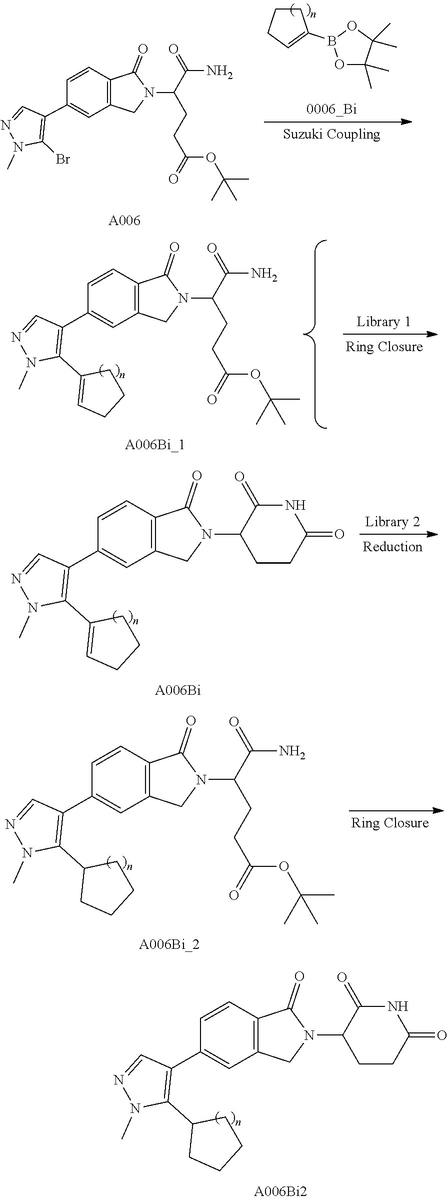

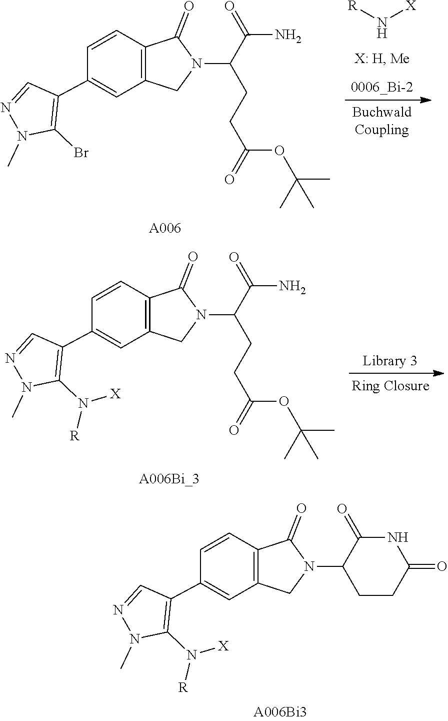

Step 1 Suzuki Coupling

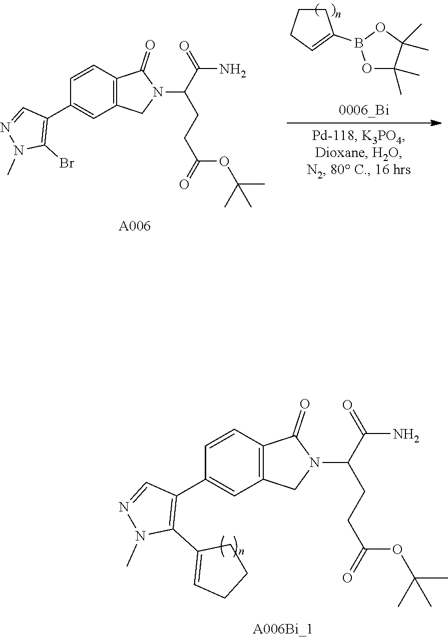

Under N₂, to a solution of A006 (143 mg, 0.30 mmol, 1.00 eq.) in Dioxane (3.00 mL) and K₃PO₄ (300 μL, 0.60 mmol, 2 M in H₂O, 2.00 eq.) was added 0006_Bi (0.45 mmol, 1.50 eq.), Pd-118 (9.75 mg, 0.015 mmol, 0.05 eq.). Then the mixture was stirred at 85° C. for 16 hrs. Spot checked by LCMS. The reaction mixture was concentrated under nitrogen flow gas, diluted with H₂O (1.00 mL) and extracted with EA (2.00 mL×3), dried over Na₂SO₄, filtered and concentrated to give A006Bi_1 used for next step.

Step 2 Ring Closure

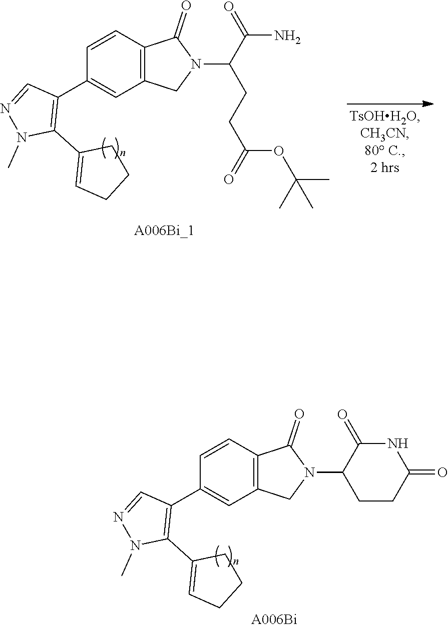

To a solution of A006Bi_1 (0.10 mmol, 1.00 eq.) in CH₃CN (1.00 mL) was added TsOH·H₂O (0.80 mmol, 8.00 eq.). Then the mixture was stirred at 80° C. for 2 hrs. Spot checked by LCMS. The reaction mixture was filtered and purified by prep-HPLC to give desired product.

Step 1 Suzuki Coupling

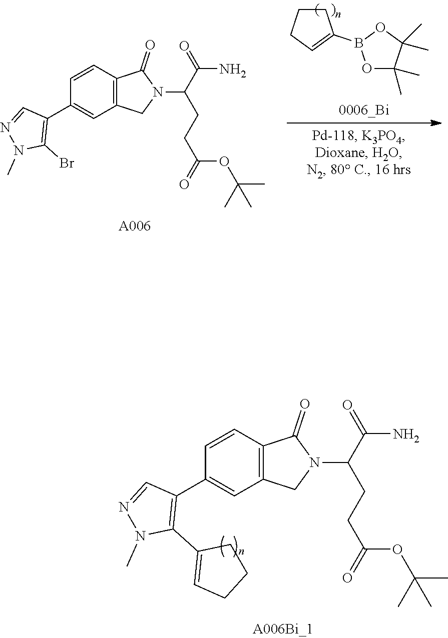

Under N₂, to a solution of A006 (143 mg, 0.30 mmol, 1.00 eq.) in Dioxane (3.00 mL) and K₃PO₄ (600 μL, 0.60 mmol, 1 M in H₂O, 2.00 eq.) was added 0006_Bi (0.45 mmol, 1.50 eq.), Pd-118 (9.75 mg, 0.015 mmol, 0.05 eq.). Then the mixture was stirred at 85° C. for 16 hrs. Spot checked by LCMS. The reaction mixture was concentrated under nitrogen flow gas, diluted with H₂O (1.00 mL) and extracted with EA (2.00 mL×3), dried over Na₂SO₄, filtered and concentrated to give A006Bi_1 used for next step.

Step 2 Hydrogenation

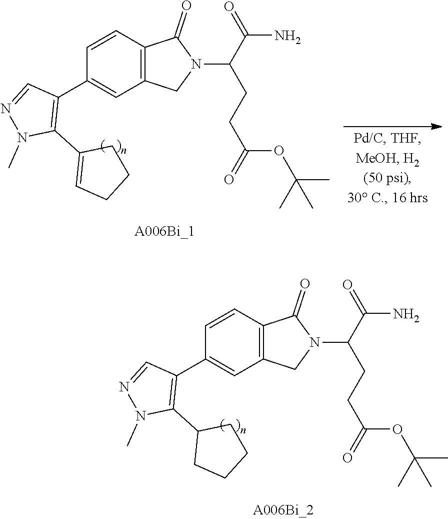

To a solution of A006Bi_1 (0.20 mmol, 1.00 eq.) in THF (1.50 mL) and MeOH (1.50 mL) was added Pd/C (10%, 120 mg, 0.60 eq.) under Ar₂ atmosphere. The suspension was degassed and purged with H₂ for 3 times. The mixture was stirred under H₂ (50 psi) at 30° C. for 16 hrs. Spot checked by LCMS. The mixture was filtered and concentrated to give A006Bi_2 used for next step.

Step 3 Ring Closure

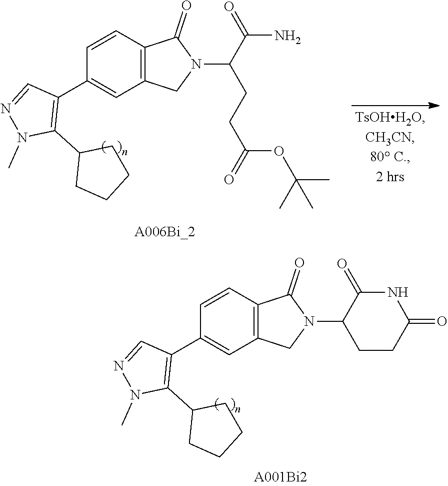

To a solution of A006Bi_2 (0.10 mmol, 1.00 eq.) in CH₃CN (1.00 mL) was added TsOH·H₂O (0.80 mmol, 8.00 eq.). Then the mixture was stirred at 80° C. for 2 hrs. Spot checked by LCMS. The reaction mixture was filtered and purified by prep-HPLC to give desired product.

Buchwald Coupling

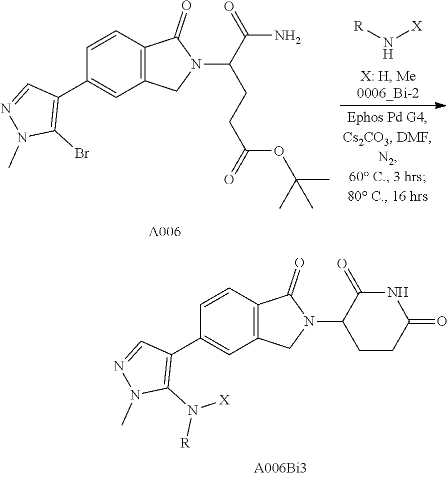

Under N₂, to a solution of A006 (143 mg, 0.30 mmol, 1.00 eq.) in DMF (3.00 mL) and Cs₂CO₃ (293 mg, 0.90 mmol, 3.00 eq.) was added 0006_Bi-2 (2.40 mmol, 8.00 eq.), EPhos Pd G₄ (27.6 mg, 0.0300 mmol, 0.100 eq.). Then the mixture was stirred at 60° C. for 3 hrs and 80° C. for 16 hrs. Spot checked by LCMS. The reaction mixture was concentrated under nitrogen flow gas, diluted with H₂O (1.00 mL) and extracted with EA (2.00 mL×3), dried over Na₂SO₄, filtered and concentrated to give a residue, which was purified by prep-HPLC to get desired product.

| Ex- | | MW + 1 |
|---|---|---|
| am- | Structure | (Ob- |
| ple | | served) |
| 588 | 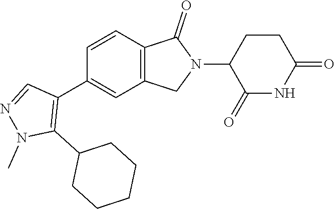 [SMILES: Cn1ncc(-c2ccc3c(c2)CN(C2CCC(=O)NC2=O)C3=O)c1C1CCCCC1] | 407.3 |
| 589 | 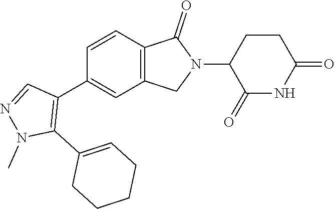 [SMILES: Cn1ncc(-c2ccc3c(c2)CN(C2CCC(=O)NC2=O)C3=O)c1C1=CCCCC1] | 405.3 |
| 590 | 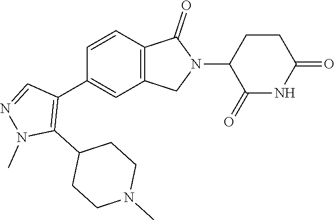 [SMILES: CN1CCC(c2c(-c3ccc4c(c3)CN(C3CCC(=O)NC3=O)C4=O)cnn2C)CC1] | 422.3 |
| 591 | 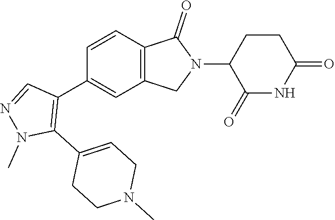 [SMILES: CN1CC=C(c2c(-c3ccc4c(c3)CN(C3CCC(=O)NC3=O)C4=O)cnn2C)CC1] | 420.3 |
| 592 | 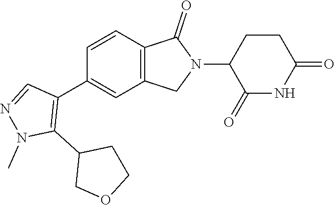 [SMILES: Cn1ncc(-c2ccc3c(c2)CN(C2CCC(=O)NC2=O)C3=O)c1C1CCOC1] | 395.2 |
| 593 | 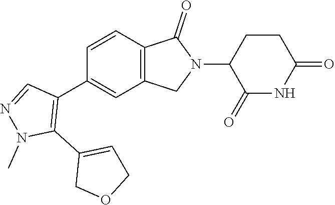 [SMILES: Cn1ncc(-c2ccc3c(c2)CN(C2CCC(=O)NC2=O)C3=O)c1C1=CCOC1] | 393.2 |

### Examples 594-598

The compounds of Examples 594-598 were prepared according to the procedure shown in Scheme 7:

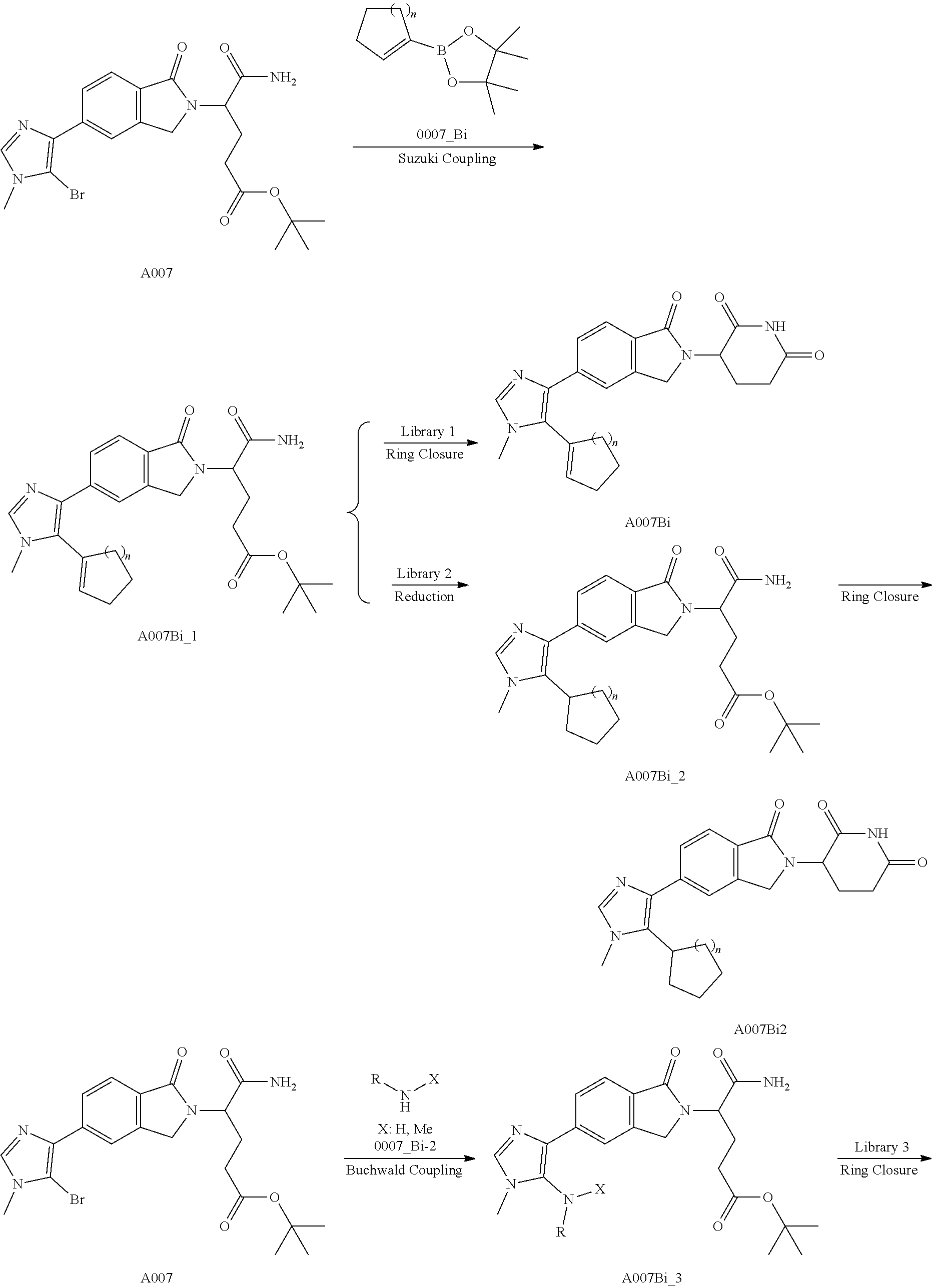

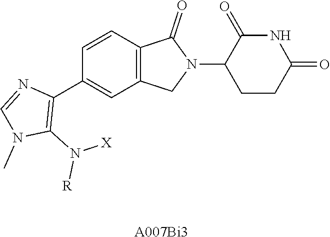

Step 1 Suzuki Coupling

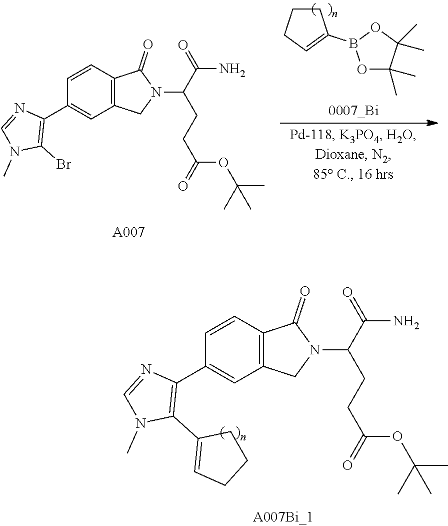

To a vial containing a solution of A007 (47.6 mg, 100 μmol, 1.00 eq) and 0007_Bi (120 μmol, 1.20 eq) in Dioxane (1.00 mL) was added K₃PO₄ (1.50 M in H₂O, 300 μmol, 3.00 eq), and Pd-118 (15.0 μmol, 0.10 eq) under protection of N₂. The mixture was stirred at 85° C. for 16 hrs. Spot checked by LCMS. The reaction mixture was diluted with H₂O (2.00 mL) and extracted with ethyl acetate (2.00 mL×3). The combined organic layers were washed with brine (2.00 mL), dried over Na₂SO₄, filtered and concentrated under reduced pressure to give crude intermediates used for next step.

Step 2 Ring Closure

To a vial containing a solution of A007Bi_1 (˜100 μmol, 1.00 eq) in CH₃CN (1.00 mL) was added TsOH·H₂O (800 μmol, 8.00 eq). The mixture was stirred at 80° C. for 2 hrs.

Spot checked by LCMS. The residue was concentrated under reduced pressure and purified by prep-HPLC to give final product.

Step 1 Suzuki Coupling

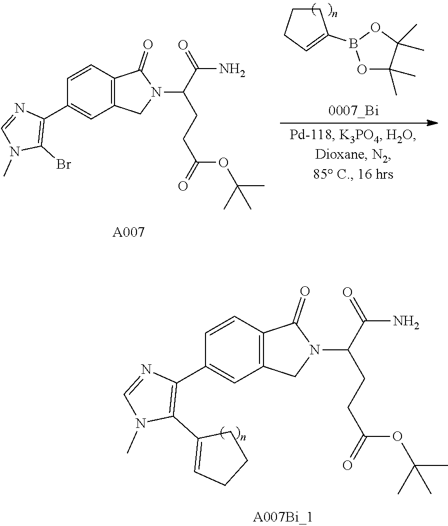

To a vial containing a solution of A007 (95.2 mg, 200 μmol, 1.00 eq) and 0007_Bi (240 μmol, 1.20 eq) in Dioxane (2.00 mL) was added K₃PO₄ (1.50 M in H₂O, 600 μmol, 3.00 eq), and Pd-118 (20.0 μmol, 0.10 eq) under protection of N₂. The mixture was stirred at 85° C. for 16 hrs. Spot checked by LCMS. The reaction mixture was diluted with H₂O (2.00 mL) and extracted with ethyl acetate (2.00 mL×3). The combined organic layers were washed with brine (2.00 mL), dried over Na₂SO₄, filtered and concentrated under reduced pressure to give crude intermediates used for next step.

Step 2 Ring Closure

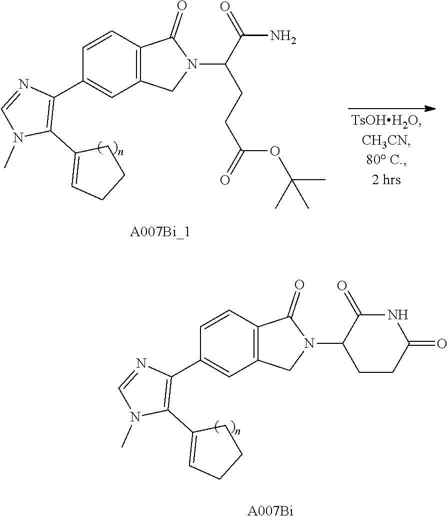

To a vial containing a solution of A007Bi_1 (˜100 μmol, 1.00 eq) in CH₃CN (1.00 mL) was added TsOH·H₂O (800 μmol, 8.00 eq). The mixture was stirred at 80° C. for 2 hrs.

Spot checked by LCMS. The residue was concentrated under reduced pressure and purified by prep-HPLC to give final product.

Step 1 Suzuki Coupling

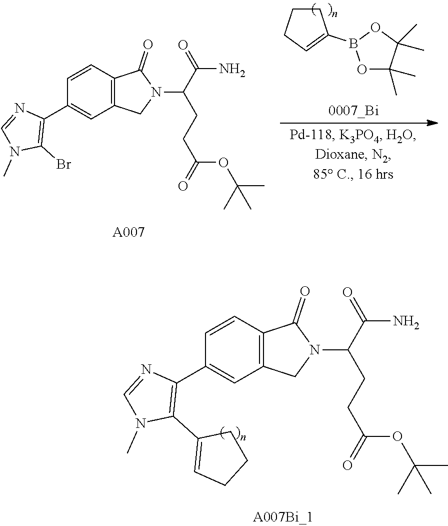

To a vial containing a solution of A007 (95.2 mg, 200 μmol, 1.00 eq) and 0007_Bi (240 μmol, 1.20 eq) in Dioxane (2.00 mL) was added K₃PO₄ (1.50 M in H₂O, 600 μmol, 3.00 eq), and Pd-118 (20.0 μmol, 0.10 eq) under protection of N₂. The mixture was stirred at 85° C. for 16 hrs. Spot checked by LCMS. The reaction mixture was diluted with H₂O (2.00 mL) and extracted with ethyl acetate (2.00 mL×3). The combined organic layers were washed with brine (2.00 mL), dried over Na₂SO₄, filtered and concentrated under reduced pressure to give crude intermediates used for next step.

Step 2 Hydrogenation

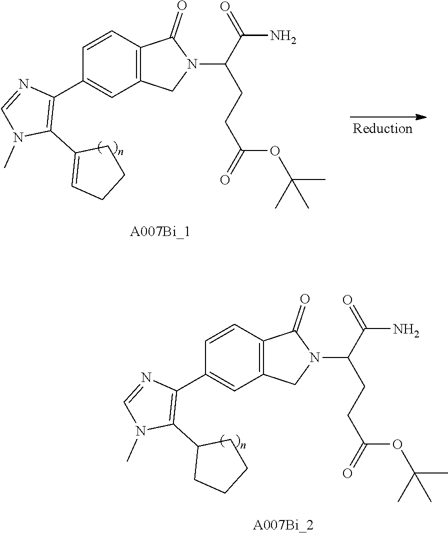

Condition 1

To a solution of A007Bi_1 (71.4 mg, 150 μmol, 1.00 eq.) in MeOH (1.50 mL) was added Pd/C (10%, 60.0 mg, 0.30 eq.) under Ar₂ atmosphere. The suspension was degassed and purged with H₂ for 3 times. The mixture was stirred under H₂ (50 psi) at 30° C. for 16 hrs. Spot checked by LCMS. The mixture was filtered and concentrated, the residue was purified by prep-HPLC.

Condition 2

To a solution of A007Bi_1 (71.4 mg, 150 μmol, 1.00 eq.) in MeOH (1.50 mL) was added Pd/C (10%, 60.0 mg, 0.30 eq.) under Ar₂ atmosphere. The suspension was degassed and purged with H₂ for 3 times. The mixture was stirred under H₂ (50 psi) at 50° C. for 16 hrs. Spot checked by LCMS. The mixture was filtered and concentrated, the residue was purified by prep-HPLC.

Step 3 Ring Closure

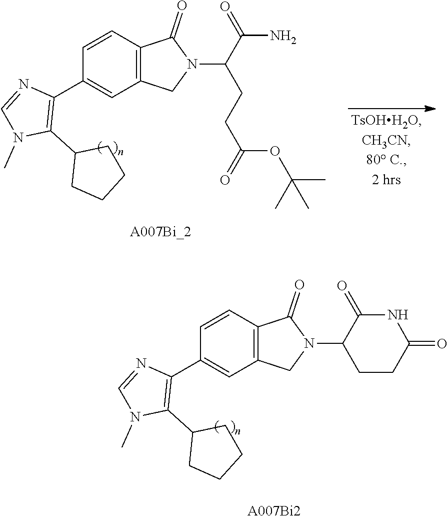

To a solution of A007Bi_2 (˜ 100 μmol, 1.00 eq) in CH₃CN (1.00 mL) was added TsOH·H₂O (800 μmol, 8.00 eq). The mixture was stirred at 80° C. for 2 hrs. Spot checked by LCMS. The residue was concentrated under reduced pressure and purified by prep-HPLC to give final product.

| Ex- | | MW + 1 |
|---|---|---|
| am- | | (Ob- |
| ple | Structure | served) |
| 594 | 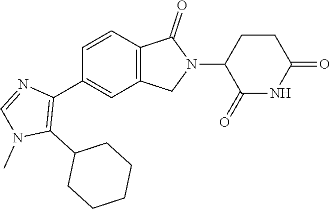 [SMILES: Cn1cnc(-c2ccc3c(c2)CN(C2CCC(=O)NC2=O)C3=O)c1C1CCCCC1] | 407.3 |
| 595 | 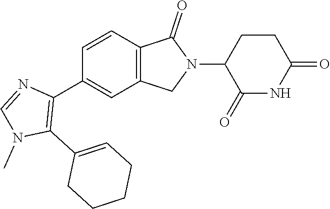 [SMILES: Cn1cnc(-c2ccc3c(c2)CN(C2CCC(=O)NC2=O)C3=O)c1C1=CCCCC1] | 405.3 |
| 596 | 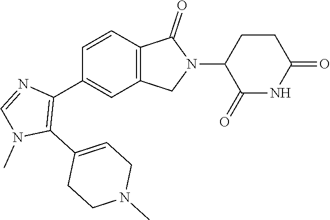 [SMILES: CN1CC=C(c2c(-c3ccc4c(c3)CN(C3CCC(=O)NC3=O)C4=O)ncn2C)CC1] | 420.3 |
| 597 | 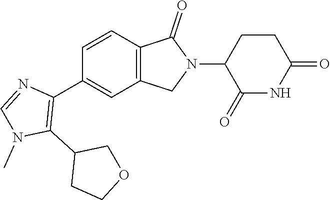 [SMILES: Cn1cnc(-c2ccc3c(c2)CN(C2CCC(=O)NC2=O)C3=O)c1C1CCOC1] | 395.3 |
| 598 | 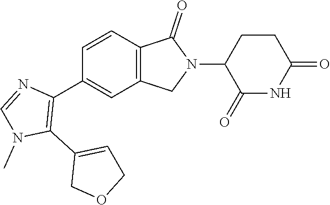 [SMILES: Cn1cnc(-c2ccc3c(c2)CN(C2CCC(=O)NC2=O)C3=O)c1C1=CCOC1] | 393.2 |

### Example 599

### Synthesis of 3-(5-(1-methyl-5-(tetrahydro-2H-pyran-4-yl)-1H-1,2,4-triazol-3-yl)-1-oxoisoindolin-2-yl)piperidine-2,6-dione

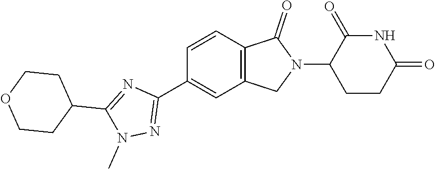 [SMILES: Cn1nc(-c2ccc3c(c2)CN(C2CCC(=O)NC2=O)C3=O)nc1C1CCOCC1]

A. 3-Bromo-5-(3,6-dihydro-2H-pyran-4-yl)-1-methyl-1H-1,2,4-triazole: To a solution of 3,5-dibromo-1-methyl-1H-1,2,4-triazole (300 mg, 1.25 mmol, 1.00 eq), 2-(3,6-dihydro-2H-pyran-4-yl)-4,4,5,5-tetramethyl-1,3,2-dioxaborolane (287 mg, 1.37 mmol, 1.10 eq) and CsF (378 mg, 2.49 mmol, 91.9 μL, 2.00 eq) in MeOH (3.00 mL) was added (PPh₃)₂PdCl₂ (87.4 mg, 124 μmol, 0.10 eq) under N₂. The mixture was stirred at 110° C. for 20 min in MW under N₂. The reaction mixture was concentrated under reduced pressure to get a residue at 45° C. The residue was purified by column chromatography (SiO₂, Petroleum ether:Ethyl acetate=100:1 to 1:1, TLC:Petroleum ether:Ethyl acetate=1:1,R_{f}=0.40) to give the title compound (140 mg, 552 μmol, 44.4% yield, 96.4% purity in LCMS at 220 nm) as off-white solid. ¹H NMR: (400 MHz, CDCl₃) δ 6.26-6.26 (m, 1H), 4.37-4.35 (m, 2H), 3.93 (s, 3H), 3.92-3.90 (m, 2H), 2.62-2.57 (m, 2H). (ESI⁺) m/z: 244.0 (M+H)⁺, (C₈H₁₀BrN₃O).

B. 3-Bromo-1-methyl-5-(tetrahydro-2H-pyran-4-yl)-1H-1,2,4-triazole: To a solution of PtO₂ (27.9 mg, 122 μmol, 1.19e-1 eq) and 3-bromo-5-(3,6-dihydro-2H-pyran-4-yl)-1-methyl-1H-1,2,4-triazole (300 mg, 1.23 mmol, 1.00 eq) in EtOH (10.0 mL) under N₂. Then the reaction mixture was degassed purged with H₂ for 3 times. Then the mixture was stirred at 25° C. for 16 h under H₂ (15 psi). The reaction mixture was filtered through diatomite and the filtrate was concentrated under vacuum to give a residue. The crude product was purified by preparative-TLC (Petroleum ether:Ethyl acetate=1:1, R_{f}=0.30) to give the title compound (105 mg, 426 μmol, 34.7% yield, 100% purity in LCMS at 220 nm) as light yellow solid. ¹H NMR: (400 MHz, DMSO-d₆) δ 3.92-3.89 (m, 2H), 3.81 (s, 3H), 3.46-3.44 (m, 2H), 3.41-3.19 (m, 1H), 1.17-1.16 (m, 4H). (ESI⁺) m/z: 245.8 (M+H)⁺, (C₈H₁₂BrN₃O).

C. 3-(5-(1-methyl-5-(tetrahydro-2H-pyran-4-yl)-1H-1,2,4-triazol-3-yl)-1-oxoisoindolin-2-yl)piperidine-2,6-dione: To a solution of 3-bromo-1-methyl-5-(tetrahydro-2H-pyran-4-yl)-1H-1,2,4-triazole (100 mg, 406 μmol, 1.00 eq), 3-(1-oxo-5-(4,4,5,5-tetramethyl-1,3,2-dioxaborolan-2-yl)isoindolin-2-yl)piperidine-2,6-dione (376 mg, 1.02 mmol, 2.00 eq) and K₃PO₄ (258 mg, 1.22 mmol, 3.00 eq) in dioxane (5.00 mL) and H₂O (0.25 mL) was added Ru-Phos-Pd-G₃ (33.9 mg, 40.6 μmol, 0.10 eq) under N₂. The mixture was stirred at 100° C. for 2 h under N₂. The reaction mixture was concentrated under reduced pressure to get a residue at 45° C. The residue was purified by preparative-HPLC (using a Phenomenex luna C18 (150×25 mm×5 μm) and gradient of 12.0%-32.0% acetonitrile in water containing 0.50% TFA over 16 min at a flow rate of 25 mL/min) to give the title compound (18.5 mg, 45.1 μmol, 11.2% yield, 99.8% purity in HPLC at 220 nm) as white solid. ¹H NMR: (400 MHz, DMSO-d₆) δ 11.0 (s, 1H), 8.29-8.25 (m, 1H), 8.18-8.11 (m, 1H), 7.79-7.77 (m, 1H), 5.15-5.12 (m, 1H), 4.54-4.42 (m, 2H), 3.97-3.93 (m, 2H), 3.91 (s, 3H), 3.50-3.47 (m, 2H), 3.27-3.24 (m, 1H), 2.91-2.89 (m, 1H), 2.63-2.58 (m, 1H), 2.39-2.30 (m, 1H), 2.07-1.99 (m, 1H), 1.83-1.79 (m, 4H). (ESI⁺) m/z: 410.0 (M+H)⁺, (C₂₁H₂₃N₅O₄).

### Example 600

### Synthesis of 3-(5-(1-methyl-5-(4-methylpiperidin-1-yl)-1H-1,2,4-triazol-3-yl)-1-oxoisoindolin-2-yl)piperidine-2,6-dione

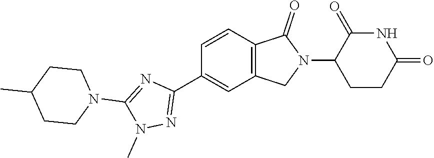 [SMILES: CC1CCN(c2nc(-c3ccc4c(c3)CN(C3CCC(=O)NC3=O)C4=O)nn2C)CC1]

A. 1-(3-Bromo-1-methyl-1H-1,2,4-triazol-5-yl)-4,4-dimethylpiperidine: A mixture of 3,5-dibromo-1-methyl-1,2,4-triazole (500 mg, 2.08 mmol, 1.00 eq), 4,4-dimethylpiperidine (234 mg, 2.08 mmol, 1.00 eq), K₂CO₃ (573 mg, 4.15 mmol, 2.00 eq) were taken up into a microwave tube in NMP (10.0 mL). The sealed tube was heated at 145° C. for 1 h under microwave. The reaction mixture was extracted with EA 60.0 mL (3×20.0 mL). The combined organic layers were washed with brine 150 mL (3×50.0 mL), dried over Na₂SO₄, filtered and concentrated under reduced pressure to give a residue. The filtrate was purified by preparative-HPLC (using a Phenomenex luna C18 (150 mm×25 mm×10 μm) and gradient of 43%-73% acetonitrile in water containing 0.05% FA over 15 min at a flow rate of 75 mL/min) to give the title compound (477 mg, 1.75 mmol, 84.1% yield, 100% purity in LCMS at 220 nm) as a white solid. ¹H NMR: (400 MHz, CDCl₃) δ 3.63 (s, 3H), 3.13-3.10 (m, 4H), 1.49-1.46 (m, 4H), 0.98 (s, 6H). (ESI⁺) m/z: 274.9 (M+3H)³⁺, (C₁₀H₁₇BrN₄).

B. 3-(5-(5-(4,4-Dimethylpiperidin-1-yl)-1-methyl-1H-1,2,4-triazol-3-yl)-1-oxoisoindolin-2-yl)piperidine-2,6-dione: A mixture of 1-(5-bromo-2-methyl-1,2,4-triazol-3-yl)-4,4-dimethyl-piperidine (477 mg, 1.75 mmol, 1.00 eq), 3-[1-oxo-5-(4,4,5,5-tetramethyl-1,3,2-dioxaborolan-2-yl)isoindolin-2-yl]piperidine-2,6-dione (1.62 g, 4.37 mmol, 2.50 eq), Ru-Phos-Pd-G₃ (146 mg, 174 μmol, 0.10 eq), K₃PO₄ (741 mg, 3.49 mmol, 2.00 eq) in dioxane (5.00 mL), H₂O (0.25 mL) was degassed and purged with N₂ for 3 times, and then the mixture was stirred at 100° C. for 0.5 h under N₂ atmosphere. The reaction mixture was extracted with EA 15.0 mL (3×5.00 mL). The combined organic layers were washed with brine 20.0 mL (2×10.0 mL), dried over Na₂SO₄, filtered and concentrated under reduced pressure to give a residue. The filtrate was purified by preparative-HPLC (using a Phenomenex luna C18 (150 mm×25 mm×10 μm) and gradient of 33%-63% acetonitrile in water containing 0.05% FA over 15 min at a flow rate of 75 mL/min) to get title compound (162 mg, 365 μmol, 20.9% yield, 98.4% purity in HPLC at 220 nm) as white solid. ¹H NMR: (400 MHz, DMSO-d₆) δ 11.0-10.9 (m, 1H), 8.12 (s, 1H), 8.07-8.05 (m, 1H), 7.77-7.75 (m, 1H), 5.16-5.09 (m, 1H), 4.52-4.35 (m, 2H), 3.71 (s, 3H), 3.18-3.16 (m, 4H), 2.92-2.91 (m, 1H), 2.62-2.58 (m, 1H), 2.45-2.35 (m, 1H), 2.04-2.00 (m, 1H), 1.49-1.47 (m, 4H), 0.99 (s, 6H). (ESI⁺) m/z: 437.0 (M+H)⁺, (C₂₃H₂₈N₆O₃).

### Example 601

### Synthesis of 3-(5-(1-methyl-2-((1r,4r)-4-(trifluoromethyl)cyclohexyl)-1H-imidazol-4-yl)-1-oxoisoindolin-2-yl)piperidine-2,6-dione

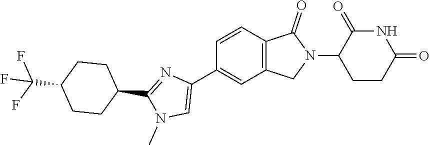 [SMILES: Cn1cc(-c2ccc3c(c2)CN(C2CCC(=O)NC2=O)C3=O)nc1[C@H]1CC[C@H](C(F)(F)F)CC1]

A. tert-Butyl 5-amino-4-(5-(N-methyl-N-((1r,4r)-4-(trifluoromethyl)cyclohexane-1-carbonyl)glycyl)-1-oxoisoindolin-2-yl)-5-oxopentanoate: To a solution of tert-butyl 5-amino-4-[5-[2-(methylamino)acetyl]-1-oxo-isoindolin-2-yl]-5-oxo-pentanoate (200 mg, 513 μmol, 1.00 eq) and 4-(trifluoromethyl)cyclohexanecarboxylic acid (120 mg, 616 μmol, 1.20 eq) in DMF (4.00 mL) was added TEA (104 mg, 1.02 mmol, 2.00 eq) at 25° C. Then the mixture was cooled down to 0° C. and T₃P (653 mg, 1.02 mmol, 50% purity, 2.00 eq) was added. Then the reaction mixture was stirred at 25° C. for 4 h. The mixture was poured into saturated NaHCO₃ (10.0 mL) and extracted with ethyl acetate (3×10.0 mL) to collect the organic layers. The combined organic layers were washed brine (3×10.0 mL), dried over Na₂SO₄, filtered and the filtrate was concentrated in vacuum to get a residue. The residue was purified by preparative-TLC (SiO₂, Dichloromethane:Methanol=10:1, R_{f}=0.50) to give the title compound (100 mg, 173 μmol, 33.7% yield, 98.3% purity in LCMS at 220 nm) as a yellow solid. (ESI⁺) m/z: 568.1 (M+H)⁺, (C₂₈H₃₆F₃N₃O₆).

B. tert-Butyl 5-amino-4-(5-(1-methyl-2-((1r,4r)-4-(trifluoromethyl)cyclohexyl)-1H-imidazol-4-yl)-1-oxoisoindolin-2-yl)-5-oxopentanoate: To a solution of tert-butyl 5-amino-4-[5-[2-[methyl-[4-(trifluoromethyl)cyclohexanecarbonyl]amino]acetyl]-1-oxo-isoindolin-2-yl]-5-oxo-pentanoate (100 mg, 173 μmol, 1.00 eq) in HCONH₂ (10.0 mL) was added NH₄OAc (66.7 mg, 865 μmol, 5.00 eq). The mixture was stirred at 100° C. for 16 h. The reaction mixture was diluted with 10.0 mL of water and extracted with EtOAc (3×10.0 mL). The combined organic layers were washed with brine (3×10.0 mL), dried over Na₂SO₄ and filtered. The filtrate was concentrated in vacuum to give the title compound (600 mg, 1.10 mmol, 60.7% yield, 98.9% purity in HPLC at 220 nm) as yellow oil without further purification. (ESI⁺) m/z: 549.1 (M+H)⁺, (C₂₈H₃₅F₃N₄O₄).

C. 3-(5-(1-Methyl-2-((1r,4r)-4-(trifluoromethyl)cyclohexyl)-1H-imidazol-4-yl)-1-oxoisoindolin-2-yl)piperidine-2,6-dione: To a solution of tert-butyl 5-amino-4-[5-[1-methyl-2-[4-(trifluoromethyl)cyclohexyl]imidazol-4-yl]-1-oxo-isoindolin-2-yl]-5-oxo-pentanoate (100 mg, 182 μmol, 1.00 eq) in ACN (3.00 mL) was added TsOH (125 mg, 729 μmol, 4.00 eq). The mixture was stirred at 80° C. for 6 h. The reaction mixture was concentrated in vacuum to get a residue. The residue was purified by preparative-HPLC (using a Phenomenex Luna C18 (100 mm×30 mm 5 μm) and gradient of 10-40% acetonitrile in water containing 0.5% FA over 8 min at a flow rate of 25.0 mL/min) to give the title compound (6.54 mg, 12.8 μmol, 7.06% yield, 93.3% purity in HPLC at 220 nm) as a white solid. ¹H NMR: (400 MHz, DMSO-d₆) δ 10.9 (s, 1H), 7.90 (s, 1H), 7.85-7.81 (m, 1H), 7.68-7.64 (m, 2H), 5.10 (dd, J=13.2, 5.2 Hz, 1H), 4.39 (dd, J=52.8, 17.2 Hz, 2H), 3.64 (s, 3H), 2.96-2.78 (m, 2H), 2.64-2.57 (m, 1H), 2.45-2.35 (m, 2H), 2.04-2.00 (m, 1H), 1.99-1.93 (m, 4H), 1.72-1.61 (m, 2H), 1.50-1.40 (m, 2H). (ESI⁺) m/z: 475.1 (M+H)⁺, (C₂₄H₂₅F₃N₄O₃).

### Example 602

### Synthesis of 3-(5-(1-methyl-5-morpholino-1H-1,2,4-triazol-3-yl)-1-oxoisoindolin-2-yl)piperidine-2,6-dione

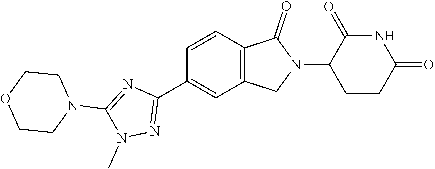 [SMILES: Cn1nc(-c2ccc3c(c2)CN(C2CCC(=O)NC2=O)C3=O)nc1N1CCOCC1]

A. 4-(3-Bromo-1-methyl-1H-1,2,4-triazol-5-yl)morpholine: To a solution of 3,5-dibromo-1-methyl-1,2,4-triazole (500 mg, 2.08 mmol, 1.00 eq) in NMP (10.0 mL) was added K₂CO₃ (573 mg, 4.15 mmol, 2.00 eq) and morpholine (180 mg, 2.08 mmol, 182 μL, 1.00 eq). The mixture was stirred in a microwave reactor at 160° C. for 45 mins. Then the reaction mixture was poured into 20.0 mL of brine and extracted with EtOAc (20.0 mL). The organic layer was washed with brine (5×20.0 mL), dried over Na₂SO₄ and filtered. The filtrate was concentrated in vacuum to get a residue. The residue was purified by preparative-TLC (SiO₂, Petroleum ether:EtOAc=1:1, R_{f}=0.25) to give the title compound (120 mg, 479 μmol, 23.0% yield, 98.7% purity in LCMS at 220 nm) as a white solid. ¹H NMR: (400 MHz, CDCl₃) δ 3.85-3.80 (m, 4H), 3.68 (s, 3H), 3.22-3.16 (m, 4H). (ESI⁺) m/z: 246.9 (M+H)⁺, (C₇H₁₁BrN₄O).

B. 3-(5-(1-Methyl-5-morpholino-1H-1,2,4-triazol-3-yl)-1-oxoisoindolin-2-yl)piperidine-2,6-dione: To a solution of 4-(5-bromo-2-methyl-1,2,4-triazol-3-yl)morpholine (120 mg, 479 μmol, 1.00 eq) and 3-[1-oxo-5-(4,4,5,5-tetramethyl-1,3,2-dioxaborolan-2-yl)isoindolin-2-yl]piperidine-2,6-dione (443 mg, 1.20 mmol, 2.50 eq) in dioxane (5.00 mL) and H₂O (0.25 mL) was added Ru-Phos-Pd-G₃ (40.0 mg, 47.9 μmol, 0.10 eq) and K₃PO₄ (203 mg, 958 μmol, 2.00 eq). The mixture was stirred at 100° C. for 2 h under N₂. Then the reaction mixture was filtered and the filtrate was concentrated in vacuum to get a residue. The residue was purified by preparative-HPLC (using a Welch Xtimate C18 (150 mm×25 mm 5 μm) and gradient of 10-40% acetonitrile in water containing 0.5% FA over 10 min at a flow rate of 25.0 mL/min) to give the title compound (38.6 mg, 93.4 μmol, 19.5% yield, 99.2% purity in HPLC at 220 nm) as a white solid. ¹H NMR: (400 MHz, DMSO-d₆) δ 11.0 (br, 1H), 8.13 (s, 1H), 8.07 (d, J=7.6 Hz, 1H), 7.77 (d, J=8.0 Hz, 1H), 5.12 (dd, J=13.2, 4.8 Hz, 1H), 4.45 (dd, J=50.0, 17.6 Hz, 2H), 3.79-3.74 (m, 7H), 3.23-3.18 (m, 4H), 2.97-2.86 (m, 1H), 2.64-2.57 (m, 1H), 2.46-2.34 (m, 1H), 2.06-1.99 (m, 1H). (ESI⁺) m/z: 411.0 (M+H)⁺, (C₂₀H₂₂N₆O₄).

### Example 603

### Synthesis of 3-(5-(5-(3,4-dihydroisoquinolin-2 (1H)-yl)-1-methyl-1H-1,2,4-triazol-3-yl)-1-oxoisoindolin-2-yl)piperidine-2,6-dione

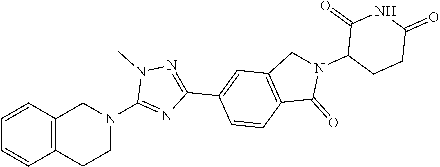 [SMILES: Cn1nc(-c2ccc3c(c2)CN(C2CCC(=O)NC2=O)C3=O)nc1N1CCc2ccccc2C1]

A. 2-(3-Bromo-1-methyl-1H-1,2,4-triazol-5-yl)-1,2,3,4-tetrahydroisoquinoline: To a solution of 3,5-dibromo-1-methyl-1H-1,2,4-triazole (400 mg, 1.66 mmol, 1.00 eq), 1,2,3,4-tetrahydroisoquinoline (221 mg, 1.66 mmol, 207 μL, 1.00 eq) in NMP (8.00 mL) was added K₂CO₃ (459 mg, 3.32 mmol, 2.00 eq). The mixture was stirred at 160° C. for 0.75 h. The mixture was poured into H₂O (30.0 mL) and extracted with Ethyl acetate (3×20.0 mL). The combined organic layer was washed with saturated NaCl aqueous (2×20.0 mL), dried over Na₂SO₄ and concentrated under reduced pressure to get residue. The residue was purified by column chromatography (SiO₂, Petroleum ether:Ethyl acetate=10:1, R_{f}=0.50) to get the title compound (272 mg, 918 μmol, 55.3% yield, 99.0% purity at 220 nm in LCMS) as yellow solid. ¹H NMR: (400 MHz, DMSO-d₆) δ 7.27-7.18 (m, 3H), 7.17-7.09 (m, 1H), 4.43 (s, 2H), 3.74 (s, 3H), 3.50-3.47 (m, 2H), 3.06-3.03 (m, 2H). (ESI⁺) m/z: 294.6 (M+H)⁺, (C₁₂H₁₃BrN₄).

B. 3-(5-(5-(3,4-Dihydroisoquinolin-2 (1H)-yl)-1-methyl-1H-1,2,4-triazol-3-yl)-1-oxoisoindolin-2-yl)piperidine-2,6-dione: To a solution of 2-(3-bromo-1-methyl-1H-1,2,4-triazol-5-yl)-1,2,3,4-tetrahydroisoquinoline (252 mg, 859 μmol, 1.00 eq), 3-(1-oxo-5-(4,4,5,5-tetramethyl-1,3,2-dioxaborolan-2-yl)isoindolin-2-yl)piperidine-2,6-dione 477 mg, 1.29 mmol, 1.50 eq) and K₃PO₄ (365 mg, 1.72 mmol, 2.00 eq) in dioxane (5.00 mL) and H₂O (0.25 mL) was added Ru-Phos-Pd-G₃ (144 mg, 171 μmol, 0.20 eq) under N₂. The mixture was stirred at 100° C. for 2 h under N₂. The mixture was filtered to collect liquid and concentrated under reduced pressure to get residue. The crude product was purified by preparative-HPLC (using a Phenomenex luna C18 (150 mm×25 mm 10 μm) and gradient of 27-57% acetonitrile in water containing 0.5% TFA over 11 min at a flow rate of 25 mL/min) to give the title compound (64.1 mg, 139 μmol, 16.2% yield, 99.2% purity in HPLC at 220 nm) as white solid. ¹H NMR: (400 MHz, DMSO-d₆) δ 10.2 (s, 1H), 8.14-8.11 (m, 1H), 8.10-8.08 (m, 1H), 7.79-7.77 (m, 1H), 7.22-7.17 (m, 4H), 5.15-5.10 (d, J=20 Hz, 1H), 4.54-4.37 (m, 4H), 3.82 (s, 3H), 3.54-3.52 (m, 2H), 3.03-3.00 (m, 2H), 2.91-2.73 (m, 1H), 2.63-2.58 (m, 1H), 2.42-2.37 (m, 1H), 2.03-2.01 (m, 1H) (ESI⁺) m/z: 456.9 (M+H)⁺, (C₂₅H₂₄N₆O₃).

### Example 604

### Synthesis of 3-(5-(5-cyclopropyl-1-methyl-2-(tetrahydro-2H-pyran-4-yl)-1H-imidazol-4-yl)-1-oxoisoindolin-2-yl)piperidine-2,6-dione

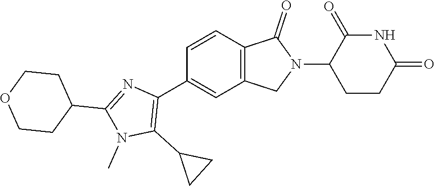 [SMILES: Cn1c(C2CCOCC2)nc(-c2ccc3c(c2)CN(C2CCC(=O)NC2=O)C3=O)c1C1CC1]

A. N-(2,2-Dimethoxyethyl)-N-methyltetrahydro-2H-pyran-4-carboximidamide: A mixture of tetrahydro-2H-pyran-4-carbonitrile (25.0 g, 225 mmol, 1.25 eq), 2,2-dimethoxy-N-methylethan-1-amine (21.4 g, 179 mmol, 23.1 mL, 1.00 eq) and CuCl (22.2 g, 224 mmol, 5.38 mL, 1.25 eq) was stirred at 85° C. for 12 h to get the title compound (40.0 g, crude) as brown oil. (ESI⁺) m/z: 230.31 (M+H)⁺, (C₁₁H₂₂N₂O₃).

B. 1-Methyl-2-(tetrahydro-2H-pyran-4-yl)-1H-imidazole: To a solution of N-(2,2-dimethoxyethyl)-N-methyl-tetrahydropyran-4-carboxamidine (20.0 g, 86.8 mmol, 1.00 eq) in MeOH (100 mL) was added HCl (12 M, 19.8 mL, 2.74 eq). The mixture was stirred at 80° C. for 24 h. The reaction mixture was concentrated under reduced pressure to give a residue. Then the residue was added 50.0% NaOH (10.0 g, aq) at 0-20° C., then added TMBE (60.0 mL) and stirred at 20° C. for 5 min. The reaction mixture was filtered to give a solid, which was washed with DCM/MeOH (10/1) (2×30.0 mL) and concentrated under reduced pressure to get residue. The residue was purified by column chromatography (SiO₂, Dichloromethane:Methanol=10:1, R_{f}=0.30) to get the title compound (4.00 g, 24.0 mmol, 13.3% yield) was obtained as brown solid. ¹H NMR: (400 MHz, DMSO-d₆) δ 6.94-6.93 (d, J=4 Hz, 1H), 6.78-6.77 (d, J=4 Hz, 1H), 4.09-4.05 (m, 2H), 3.60 (s, 3H), 3.60-3.49 (m, 2H), 2.89-2.85 (m, 1H), 2.05-2.00 (m, 2H), 1.85-1.75 (m, 2H). (ESI⁺) m/z: 166.22 (M+H)⁺, (C₉H₁₄N₂O).

C. 5-Bromo-1-methyl-2-(tetrahydro-2H-pyran-4-yl)-1H-imidazole: To a solution of 5-bromo-1-methyl-2-(tetrahydro-2H-pyran-4-yl)-1H-imidazole (1.50 g, 9.02 mmol, 1.00 eq) in DMF (15.0 mL) was added NBS (1.45 g, 8.12 mmol, 0.90 eq) dropwise at 0° C. The mixture was stirred at 0° C. for 2 h. The mixture was poured into H₂O (30.0 mL) and extracted with Ethyl acetate (3×20.0 mL). The combined organic layer was washed with saturated NaCl aqueous (2×20.0 mL), dried over Na₂SO₄ and concentrated under reduced pressure to get residue. The residue was purified by column chromatography (SiO₂, Dichloromethane:Methanol=10:1, R_{f}=0.50) to give the title compound (1.80 g, 7.34 mmol, 81.3% yield) as yellow solid. ¹H NMR: (400 MHz, DMSO-d₆) δ 6.88 (s, 1H), 3.91-3.88 (m, 2H), 3.53 (s, 3H), 3.53-3.43 (m, 2H), 3.31-3.30 (m, 1H), 1.72-1.67 (m, 4H). (ESI⁺) m/z: 245.12 (M+H)⁺, (C₉H₁₃BrN₂O).

D. 5-Cyclopropyl-1-methyl-2-(tetrahydro-2H-pyran-4-yl)-1H-imidazole: To a solution of 5-bromo-1-methyl-2-(tetrahydro-2H-pyran-4-yl)-1H-imidazole (900 mg, 3.67 mmol, 1.00 eq), cyclopropylboronic acid (946 mg, 11.0 mmol, 3.00 eq) and K₃PO₄ (1.56 g, 7.34 mmol, 2.00 eq) in dioxane (9.00 mL) and H₂O (0.90 mL) was added Pd(PPh₃)₄ (424 mg, 367 μmol, 0.10 eq) under N₂. The mixture was stirred at 90° C. for 36 h under N₂. The mixture was filtered to collect liquid and concentrated under reduced pressure to get residue. The crude product was purified by preparative-HPLC (using a Phenomenex luna C18 (150 mm×25 mm 10 μm) and gradient of 5-40% acetonitrile in water containing 0.05% NH₃H₂O over 15 min at a flow rate of 25 mL/min) to give the title compound (800 mg, 3.88 mmol, 52.8% yield) as white solid. ¹H NMR: (400 MHz, DMSO-d₆) δ 6.59 (s, 1H), 4.09-4.05 (m, 2H), 3.55 (s, 3H), 3.54-3.49 (m, 2H), 2.88-2.83 (m, 1H), 2.06-2.00 (m, 2H), 1.79-1.76 (m, 2H), 1.58-1.54 (m, 1H), 0.88-0.85 (m, 2H), 0.60-0.58 (m, 2H). (ESI⁺) m/z: 207.14 (M+H)⁺, (C₁₂H₁₈N₂O).

E. 4-Bromo-5-cyclopropyl-1-methyl-2-(tetrahydro-2H-pyran-4-yl)-1H-imidazole: To a solution of 5-cyclopropyl-1-methyl-2-(tetrahydro-2H-pyran-4-yl)-1H-imidazole (600 mg, 2.91 mmol, 1.00 eq) in DMF (6.00 mL) was added NBS (543 mg, 3.05 mmol, 1.05 eq) dropwise at 0° C. The mixture was stirred at 25° C. for 2 h. The mixture was poured into H₂O (30.0 mL) and extracted with Ethyl acetate (3×20.0 mL). The combined organic layer was washed with saturated NaCl aqueous (2×20.0 mL), dried over Na₂SO₄ and concentrated under reduced pressure to get residue. The residue was purified by preparative-TLC (Dichloromethane:Methanol=10:1, R_{f}=0.50) to give the title compound (263 mg, 890 μmol, 30.6% yield, 96.6% purity in LCMS at 220 nm) as yellow solid. ¹H NMR: (400 MHz, DMSO-d₆) δ 4.08-4.04 (m, 2H), 3.60 (s, 3H), 3.53-3.47 (m, 2H), 2.87-2.81 (m, 1H), 2.05-2.02 (m, 2H), 1.76-1.72 (m, 2H), 1.50-1.47 (m, 1H), 0.99-0.95 (m, 2H), 0.80-0.78 (m, 2H). (ESI⁺) m/z: 286.9 (M+H)⁺, (C₁₂H₁₇BrN₂O).

F. 3-(5-(5-Cyclopropyl-1-methyl-2-(tetrahydro-2H-pyran-4-yl)-1H-imidazol-4-yl)-1-oxoisoindolin-2-yl)piperidine-2,6-dione: To a solution of 4-bromo-5-cyclopropyl-1-methyl-2-(tetrahydro-2H-pyran-4-yl)-1H-imidazole (263 mg, 922 μmol, 1.00 eq), 3-(1-oxo-5-(4,4,5,5-tetramethyl-1,3,2-dioxaborolan-2-yl)isoindolin-2-yl)piperidine-2,6-dione (853 mg, 2.31 mmol, 2.50 eq) and K₃PO₄ (391 mg, 1.84 mmol, 2.00 eq) in dioxane (5.00 mL) and H₂O (0.25 mL) was added Ru-Phos-Pd-G₃ (154 mg, 184 μmol, 0.20 eq) under N₂. The mixture was stirred at 100° C. for 2 h under N₂. The mixture was filtered to collect liquid and concentrated under reduced pressure to get residue. The crude product was purified by preparative-HPLC (using a Phenomenex luna C18 (150 mm×25 mm 10 μm) and gradient of 8-28% acetonitrile in water containing 0.5% TFA over 11 min at a flow rate of 25 mL/min) to give the title compound (186 mg, 414 μmol, 44.9% yield, 100% purity in HPLC at 220 nm) as white solid. ¹H NMR: (400 MHz, DMSO-d₆) δ 11.02 (s, 1H), 7.90-7.89 (m, 2H), 7.81-7.79 (m, 1H), 5.17-5.14 (m, 1H), 4.57-4.40 (m, 2H), 3.99-3.97 (m, 2H), 3.87 (s, 3H), 3.52-3.47 (m, 3H), 2.93-2.87 (m, 1H), 2.64-2.60 (m, 1H), 2.03-2.00 (m, 1H), 1.95-1.85 (m, 6H), 1.10-1.05 (m, 2H), 0.38-0.34 (m, 2H). (ESI⁺) m/z: 449.1 (M+H)⁺, (C₂₅H₂₈N₄O₄).

### Example 605

### Synthesis of 3-(5-(5-(4,4-dimethylcyclohexyl)-1-methyl-1H-1,2,4-triazol-3-yl)-1-oxoisoindolin-2-yl)piperidine-2,6-dione

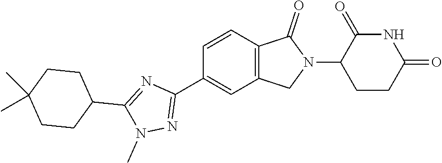 [SMILES: Cn1nc(-c2ccc3c(c2)CN(C2CCC(=O)NC2=O)C3=O)nc1C1CCC(C)(C)CC1]

A. 3-Bromo-5-(3,6-dihydro-2H-pyran-4-yl)-1-methyl-1H-1,2,4-triazole: To a solution of 3,5-dibromo-1-methyl-1H-1,2,4-triazole (300 mg, 1.25 mmol, 1.00 eq), 2-(4,4-dimethylcyclohex-1-en-1-yl)-4,4,5,5-tetramethyl-1,3,2-dioxaborolane (323 mg, 1.37 mmol, 1.10 eq) and CsF (378 mg, 2.49 mmol, 91.9 μL, 2.00 eq) in MeOH (3.00 mL) was added (PPh₃)₂PdCl₂ (87.4 mg, 124 μmol, 0.10 eq) under N₂. The mixture was stirred at 110° C. for 20 min in MW under N₂. The reaction mixture was concentrated under reduced pressure to get a residue at 45° C. The residue was purified by column chromatography (SiO₂, Petroleum ether:Ethyl acetate=100:1 to 3:1, TLC:Petroleum ether:Ethyl acetate=3:1,R_{f}=0.50) to give the title compound (167 mg, 493 μmol, 39.6% yield, 96.9% purity in LCMS at 220 nm) as white solid. ¹H NMR: (400 MHz, CDCl₃) δ 6.14-6.11 (m, 1H), 3.87 (s, 3H), 2.48-2.44 (m, 2H), 2.06-2.04 (m, 2H), 1.54-1.50 (m, 2H), 0.99 (s, 6H). (ESI⁺) m/z: 270.0 (M+H)⁺, (C₁₁H₁₆BrN₃).

B. 3-Bromo-5-(4, 4-dimethylcyclohexyl)-1-methyl-1H-1,2,4-triazole: To a solution of PtO₂ (40.0 mg, 176 μmol, 1.19e-1 eq) and 3-bromo-5-(4,4-dimethylcyclohex-1-en-1-yl)-1-methyl-1H-1,2,4-triazole (400 mg, 1.48 mmol, 1.00 eq) in EtOH (10.0 mL) under N₂. Then the reaction mixture was degassed purged with H₂ for 3 times. Then the mixture was stirred at 25° C. for 16 h under H₂ (15 psi). The reaction mixture was filtered through diatomite and the filtrate was concentrated under vacuum to give a residue. The crude product was purified by preparative-TLC (Petroleum ether:Ethyl acetate=3:1, R_{f}=0.30) to give the title compound (90.0 mg, 329 μmol, 20.0% yield, 100% purity in LCMS at 220 nm) as light yellow oil. (ESI⁺) m/z: 272.0 (M+H)⁺, (C₁₁H₁₈BrN₃).

C. 3-(5-(5-(4,4-Dimethylcyclohexyl)-1-methyl-1H-1,2,4-triazol-3-yl)-1-oxoisoindolin-2-yl)piperidine-2,6-dione: To a solution of 3-bromo-5-(4,4-dimethylcyclohexyl)-1-methyl-1H-1,2,4-triazole (90.0 mg, 329 μmol, 1.00 eq), 3-(1-oxo-5-(4,4,5,5-tetramethyl-1,3,2-dioxaborolan-2-yl)isoindolin-2-yl)piperidine-2,6-dione (458 mg, 1.15 mmol, 3.50 eq) and K₃PO₄ (210 mg, 0.99 mmol, 3.00 eq) in dioxane (5.00 mL) and H₂O (0.25 mL) was added Ru-Phos-Pd-G₃ (27.6 mg, 32.9 μmol, 0.10 eq) under N₂. The mixture was stirred at 100° C. for 2 h under N₂. The reaction mixture was concentrated under reduced pressure to get a residue at 45° C. The residue was purified by preparative-HPLC (using a Phenomenex luna C18 (150×25 mm×5 μm) and gradient of 37.0%-57.0% acetonitrile in water containing 0.50% TFA over 16 min at a flow rate of 25 mL/min) to give the title compound (14.1 mg, 32.1 μmol, 10.1% yield, 99.8% purity in HPLC at 220 nm) as white solid. ¹H NMR: (400 MHz, DMSO-d₆) δ 11.0 (s, 1H), 8.26-8.23 (m, 1H), 8.18-8.10 (m, 1H), 7.79-7.77 (m, 1H), 5.15-5.10 (m, 1H), 4.54-4.37 (m, 2H), 3.88 (s, 3H), 2.92-2.85 (m, 2H), 2.63-2.52 (m, 1H), 2.47-2.42 (m, 1H), 2.09-1.99 (m, 1H), 1.76-1.72 (m, 4H), 1.50-1.47 (m, 2H), 1.37-1.30 (m, 2H), 0.97-0.96 (m, 6H). (ESI⁺) m/z: 436.2 (M+H)⁺, (C₂₄H₂₉N₅O₃).

### Example 606

### Synthesis of 3-(5-(5-(4,4-difluorocyclohexyl)-1-methyl-1H-1,2,4-triazol-3-yl)-1-oxoisoindolin-2-yl)piperidine-2,6-dione

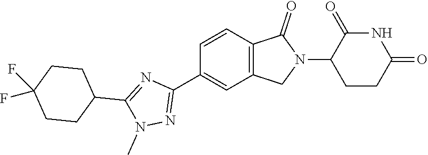 [SMILES: Cn1nc(-c2ccc3c(c2)CN(C2CCC(=O)NC2=O)C3=O)nc1C1CCC(F)(F)CC1]

A. 3-Bromo-5-(4,4-difluorocyclohex-1-en-1-yl)-1-methyl-1H-1,2,4-triazole: To a solution of 3,5-dibromo-1-methyl-1H-1,2,4-triazole (300 mg, 1.25 mmol, 1.00 eq), 2-(4,4-difluorocyclohex-1-en-1-yl)-4,4,5,5-tetramethyl-1,3,2-dioxaborolane (334 mg, 1.37 mmol, 1.10 eq) and CsF (378 mg, 2.49 mmol, 91.9 μL, 2.00 eq) in MeOH (3.00 mL) was added (PPh₃)₂PdCl₂ (87.4 mg, 124 μmol, 0.10 eq) under N₂. The mixture was stirred at 110° C. for 20 min in MW under N₂. The reaction mixture was concentrated under reduced pressure to get a residue at 45° C. The residue was purified by column chromatography (SiO₂, Petroleum ether:Ethyl acetate=100:1 to 3:1, TLC:Petroleum ether:Ethyl acetate=3:1,R_{f}=0.60) to give the title compound (226 mg, 729 μmol, 58.6% yield, 95.8% purity in LCMS at 220 nm) as white solid. ¹H NMR: (400 MHz, CDCl₃) δ 6.05 (s, 1H), 3.96 (s, 3H), 2.78-2.75 (m, 4H), 2.24-2.15 (m, 2H). (ESI⁺) m/z: 280.0 (M+H)⁺, (C₉H₁₀BrF₂N₃).

B. 3-Bromo-5-(4, 4-difluorocyclohexyl)-1-methyl-1H-1,2,4-triazole: To a solution of PtO₂ (40.0 mg, 176 μmol, 1.19e-1 eq) and 3-bromo-5-(4,4-difluorocyclohex-1-en-1-yl)-1-methyl-1H-1,2,4-triazole (400 mg, 1.44 mmol, 1.00 eq) in EtOH (10.0 mL) under N₂. Then the reaction mixture was degassed purged with H₂ for 3 times. Then the mixture was stirred at 25° C. for 16 h under H₂ (15 psi). The reaction mixture was filtered through diatomite and the filtrate was concentrated under vacuum to give a residue. The crude product was purified by preparative-TLC (Petroleum ether:Ethyl acetate=1:1, R_{f}=0.30) to give the title compound (150 mg, 535 μmol, 30.0% yield, 100% purity in LCMS at 220 nm) as light yellow solid. (ESI⁺) m/z: 282.0 (M+H)⁺, (C₉H₁₂BrF₂N₃).

C. 3-(5-(5-(4,4-Difluorocyclohexyl)-1-methyl-1H-1,2,4-triazol-3-yl)-1-oxoisoindolin-2-yl)piperidine-2,6-dione: To a solution of 3-bromo-5-(4,4-difluorocyclohexyl)-1-methyl-1H-1,2,4-triazole (150 mg, 535 μmol, 1.00 eq), 3-(1-oxo-5-(4,4,5,5-tetramethyl-1,3,2-dioxaborolan-2-yl)isoindolin-2-yl)piperidine-2,6-dione (594 mg, 1.61 mmol, 3.00 eq) and K₃PO₄ (340 mg, 1.61 mmol, 3.00 eq) in dioxane (5.00 mL) and H₂O (0.25 mL) was added Ru-Phos-Pd-G₃ (44.1 mg, 53.4 μmol, 0.10 eq) under N₂. The mixture was stirred at 100° C. for 2 h under N₂. The reaction mixture was concentrated under reduced pressure to get a residue at 45° C. The residue was purified by preparative-HPLC (using a Phenomenex luna C18 (150×25 mm×5 μm) and gradient of 24.0%-44.0% acetonitrile in water containing 0.50% TFA over 16 min at a flow rate of 25 mL/min) to give the title compound (28.0 mg, 61.5 μmol, 11.8% yield, 97.5% purity in HPLC at 220 nm) as white solid. ¹H NMR: (400 MHz, DMSO-d₆) δ 11.0 (s, 1H), 8.26-8.23 (m, 1H), 8.18-8.10 (m, 1H), 7.81-7.77 (m, 1H), 5.15-5.10 (m, 1H), 4.53-4.37 (m, 2H), 3.91 (s, 3H), 3.22-3.19 (m, 1H), 2.98-2.92 (m, 1H), 2.63-2.58 (m, 1H), 2.48-2.38 (m, 1H), 2.18-2.16 (m, 1H), 2.15-2.03 (m, 6H), 1.99-1.82 (m, 2H), (ESI⁺) m/z: 444.2 (M+H)⁺, (C₂₂H₂₃F₂N₅O₃).

### Example 607

### Synthesis of 3-(1-oxo-5-(5-phenyl-1H-imidazol-1-yl)isoindolin-2-yl)piperidine-2,6-dione

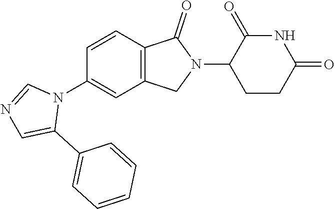 [SMILES: O=C1CCC(N2Cc3cc(-n4cncc4-c4ccccc4)ccc3C2=O)C(=O)N1]

A. (2-(1-Amino-5-(tert-butoxy)-1,5-dioxopentan-2-yl)-1-oxoisoindolin-5-yl)boronic acid: A mixture of tert-butyl 5-amino-5-oxo-4-[1-oxo-5-(4,4,5,5-tetramethyl-1,3,2-dioxaborolan-2-yl)isoindolin-2-yl]pentanoate (1.50 g, 3.38 mmol, 1.00 eq) in THF (10.0 mL) and H₂O (5.00 mL), was added NaNO₂ (233 mg, 3.38 mmol, 1.00 eq) to the reaction mixture slowly. Then the reaction mixture was stirred at 25° C. for 12 h. The reaction mixture was poured into 20.0 ml H₂O, extracted with DCM (20.0 ml*3), concentrated to give to give the title compound (950 mg, 2.62 mmol, 77.7% yield). (C₁₇H₂₃BN₂O₆).

B. tert-Butyl 5-amino-5-oxo-4-(1-oxo-5-(5-phenyl-1H-imidazol-1-yl)isoindolin-2-yl)pentanoate: A mixture of 4-phenyl-1H-imidazole (200 mg, 1.39 mmol, 1.00 eq) and [2-(2,6-dioxo-3-piperidyl)-1-oxo-isoindolin-5-yl]boronic acid (400 mg, 1.39 mmol, 1.00 eq) in DCM (5.00 mL), was added chloro(hydroxy)copper; N,N,N′,N′-tetramethylethane-1,2-diamine (64.5 mg, 138.86 μmol, 0.10 eq) to the reaction mixture, the mixture was stirred at 25° C. for 12 h under 02 (15 psi). The residue was purified by preparative-TLC (SiO₂, Dichloromethane:Methanol=20:1; TLC, Dichloromethane:Methanol=20:1, R_{f}=0.40) to give the title compound (30.0 mg, 65.2 μmol, 7.8% yield). (C₂₆H₂₈N₄O₄).

C. 3-(1-Oxo-5-(5-phenyl-1H-imidazol-1-yl)isoindolin-2-yl)piperidine-2,6-dione: A mixture of tert-butyl 5-amino-5-oxo-4-[1-oxo-5-(5-phenylimidazol-1-yl)isoindolin-2-yl]pentanoate (30.0 mg, 65.1 μmol, 1.00 eq) and TsOH (44.9 mg, 261 μmol, 4.00 eq) in ACN (5.00 mL) was stirred at 70° C. for 4 h. The reaction mixture was purified by preparative-HPLC (using a Welch Xtimate C18 (150 mm×25 mm×10 μm) and gradient of 23-53% acetonitrile in water containing 0.05% TFA over 10 min at a flow rate of 25.0 mL/min) to give the title compound (15.0 mg, 38.3 μmol, 14.8% yield, 98.7% purity in HPLC at 220 nm) as a white solid. ¹H NMR: (400 MHz, DMSO-d₆) δ 11.0 (s, 1H), 8.02 (s, 1H), 7.76 (d, J=8.0 Hz, 1H), 7.64 (s, 1H), 7.36-7.26 (m, 5H), 7.18-7.16 (m, 2H), 5.13 (dd, J=13.6, 5.2 Hz, 1H), 4.41 (dd, J=50.0, 17.6 Hz, 2H), 2.96-2.86 (m, 1H), 2.62-2.57 (m, 1H), 2.43-2.38 (m, 1H), 2.04-2.00 (m, 1H). (ESI⁺) m/z: 387.1 (M+H)⁺, (C₂₂H₁₈N₄O₃).

### Example 608

### Synthesis of 3-(5-(1-methyl-5-(6-azaspiro[2.5]octan-6-yl)-1H-1,2,4-triazol-3-yl)-1-oxoisoindolin-2-yl)piperidine-2,6-dione

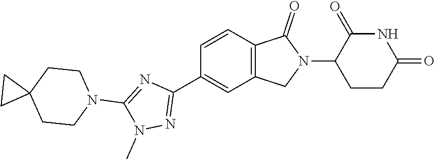 [SMILES: Cn1nc(-c2ccc3c(c2)CN(C2CCC(=O)NC2=O)C3=O)nc1N1CCC2(CC1)CC2]

A. 6-(3-Bromo-1-methyl-1H-1,2,4-triazol-5-yl)-6-azaspiro[2.5]octane: A mixture of 3,5-dibromo-1-methyl-1,2,4-triazole (100 mg, 415 μmol, 1.00 eq), 6-azaspiro[2.5]octane (46.1 mg, 415 μmol, 1.00 eq), and K₂CO₃ (114 mg, 830 μmol, 2.00 eq) were taken up into a microwave tube in NMP (2.00 mL). The sealed tube was heated at 145° C. for 45 min under microwave. The reaction mixture was extracted with EA 15 mL (3×5.00 mL). The combined organic layers were washed with brine 30.0 mL (3×10.0 mL), dried over Na₂SO₄, filtered and concentrated under reduced pressure to give a residue to give the title compound (69.0 mg, 246 μmol, 59.3% yield, 96.8% purity in LCMS at 220 nm) as a black brown solid. ¹H NMR: (400 MHz, CDCl₃) δ 3.70-3.64 (m, 3H), 3.21-3.19 (m, 4H), 1.52-1.49 (m, 4H), 0.36 (s, 4H). (ESI⁺) m/z: 271.0 (M+H)⁺, (C₁₀H₁₅BrN₄).

B. 3-(5-(1-Methyl-5-(6-azaspiro[2.5]octan-6-yl)-1H-1,2,4-triazol-3-yl)-1-oxoisoindolin-2-yl)piperidine-2,6-dione: A mixture of 6-(5-bromo-2-methyl-1,2,4-triazol-3-yl)-6-azaspiro[2.5]octane (69.0 mg, 246 μmol, 1.00 eq), 3-[1-oxo-5-(4,4,5,5-tetramethyl-1,3,2-dioxaborolan-2-yl)isoindolin-2-yl]piperidine-2,6-dione (227 mg, 615 μmol, 2.50 eq), Ru-Phos-Pd-G₃ (20.6 mg, 24.6 μmol, 0.10 eq), K₃PO₄ (104 mg, 492 μmol, 2.00 eq) in dioxane (1.00 mL), H₂O (0.05 mL) was degassed and purged with N₂ for 3 times, and then the mixture was stirred at 100° C. for 0.5 h under N₂ atmosphere. The reaction mixture was extracted with EA 15.0 mL (3×5.00 mL). The combined organic layers were washed with brine 20.0 mL (2×10.0 mL), dried over Na₂SO₄, filtered and concentrated under reduced pressure to give a residue. The filtrate was purified by preparative-HPLC (using a Phenomenex luna C18 (150 mm×25 mm×5 μm) and gradient of 30%-50% acetonitrile in water containing 0.05% TFA over 15 min at a flow rate of 25 mL/min) to get title compound (16.2 mg, 36.6 μmol, 14.8% yield, 98.1% purity in HPLC at 220 nm) as off-white solid. ¹H NMR: (400 MHz, DMSO-d₆) δ 11.0 (s, 1H), 8.13 (s, 1H), 8.08-8.06 (s, 1H), 7.78-7.76 (m, 1H), 5.17-5.08 (m, 1H), 4.56-4.34 (m, 2H), 3.74 (s, 3H), 3.23-3.21 (m, 4H), 2.96-2.87 (m, 1H), 2.62-2.58 (m, 1H), 2.42-2.37 (m, 1H), 2.06-1.97 (m, 1H), 1.55-1.46 (m, 4H), 0.36 (s, 4H). (ESI⁺) m/z: 435.2 (M+H)⁺, (C₂₃H₂₆N₆O₃).

### Example 609

### Synthesis of 3-(5-(5-(isoindolin-2-yl)-1-methyl-1H-1,2,4-triazol-3-yl)-1-oxoisoindolin-2-yl)piperidine-2,6-dione

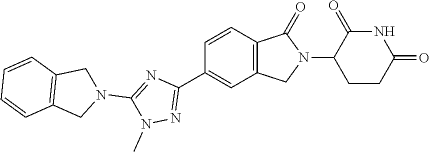 [SMILES: Cn1nc(-c2ccc3c(c2)CN(C2CCC(=O)NC2=O)C3=O)nc1N1Cc2ccccc2C1]

A. 2-(3-Bromo-1-methyl-1H-1,2,4-triazol-5-yl)isoindoline: To a solution of 3,5-dibromo-1-methyl-1H-1,2,4-triazole (400 mg, 1.66 mmol, 1.00 eq), isoindoline (197 mg, 1.66 mmol, 188 μL, 1.00 eq) and K₂CO₃ (458 mg, 3.32 mmol, 2.00 eq) in NMP (8.00 mL). The mixture was stirred at 160° C. for 45 min in MW under N₂. The reaction mixture was poured into H₂O (30.0 mL) and extracted with Ethyl acetate (3×30.0 mL). The combined organic layer was washed with brine (3×30.0 mL), dried over Na₂SO₄, filtered and concentrated. The residue was purified by preparative-TLC (Petroleum ether:Ethyl acetate=1:1, R_{f}=0.40) to give the title compound (390 mg, 1.40 mmol, 84.10% yield, 100% purity in LCMS at 220 nm) as yellow solid. ¹H NMR: (400 MHz, DMSO-d₆) δ7.37-7.30 (m, 4H), 4.90 (s, 4H), 3.85 (s, 3H). (ESI⁺) m/z: 278.8 (M+H)⁺, (C₁₁H₁₁BrN₄).

B. 3-(5-(5-(Isoindolin-2-yl)-1-methyl-1H-1,2,4-triazol-3-yl)-1-oxoisoindolin-2-yl)piperidine-2,6-dione: To a solution of 2-(3-bromo-1-methyl-1H-1,2,4-triazol-5-yl)isoindoline (370 mg, 1.33 mmol, 1.00 eq), 3-(1-oxo-5-(4,4,5,5-tetramethyl-1,3,2-dioxaborolan-2-yl)isoindolin-2-yl)piperidine-2,6-dione (1.23 g, 3.31 mmol, 2.50 eq) and K₃PO₄ (844 mg, 3.98 mmol, 3.00 eq) in dioxane (10.0 mL) and H₂O (0.50 mL) was added Ru-Phos-Pd-G₃ (110 mg, 132 μmol, 0.10 eq) under N₂. The mixture was stirred at 100° C. for 3 h under N₂. The reaction mixture was concentrated under reduced pressure to get a residue at 45° C. The residue was purified by preparative-HPLC (using a Phenomenex luna C18 (200×40 mm×10 μm) and gradient of 13.0%-43.0% acetonitrile in water containing 0.50% TFA over 14 min at a flow rate of 25 mL/min) to give the title compound (199 mg, 446 μmol, 33.7% yield, 99.2% purity in HPLC at 220 nm) as white solid. ¹H NMR: (400 MHz, DMSO-d₆) δ 11.03-11.00 (m, 1H), 8.20-8.13 (m, 2H), 7.79-7.75 (m, 1H), 7.41-7.31 (m, 4H), 5.14-5.10 (m, 1H), 5.00 (s 4H), 4.54-4.37 (m, 2H), 3.98-3.97 (m, 3H), 2.96-2.91 (m, 1H), 2.63-2.59 (m, 1H), 2.42-2.38 (m, 1H), 2.04-2.02 (m, 1H). (ESI⁺) m/z: 443.2 (M+H)⁺, (C₂₄H₂₂N₆O₃).

### Example 610

### Synthesis of 3-(5-(1-isopropyl-3-phenyl-1H-1,2,4-triazol-5-yl)-1-oxoisoindolin-2-yl)piperidine-2,6-dione

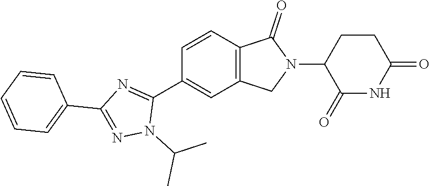 [SMILES: CC(C)n1nc(-c2ccccc2)nc1-c1ccc2c(c1)CN(C1CCC(=O)NC1=O)C2=O]

A. 4-Bromo-1-isopropyl-5-phenyl-1H-pyrazole: To a solution of 3,5-dibromo-1H-1,2,4-triazole (10.0 g, 44.0 mmol, 1.00 eq) in DMF (100 mL) was added NaH (2.64 g, 66.1 mmol, 60% purity, 1.50 eq) at 0° C. under N₂. The mixture was stirred at 25° C. under N₂ for 0.5 h. Then the mixture was added 2-iodopropane (14.9 g, 88.1 mmol, 8.80 mL, 2.00 eq) at 25° C. under N₂. The mixture was stirred at 25° C. for 11.5 h under N₂. After the reaction was completed, the reaction mixture was poured into saturated NH₄Cl aqueous solution (200 mL) and extracted with ethyl acetate (3×100 mL). The combined organic layer was washed with brine (100 mL), dried over Na₂SO₄, filtered and the filtrate was concentrated in vacuum to get a residue. The residue was purified by column chromatography (SiO₂, Petroleum ether: Ethyl acetate=100:1 to 5:1,R_{f}=0.50 (Petroleum ether:Ethyl acetate=5:1)) to give the title compound (10.0 g, 36.8 mmol, 84.3% yield, 99.2% purity in HPLC at 220 nm) as white oil. ¹H NMR: (400 MHz, DMSO-d₆) δ 4.72-4.64 (m, 1H), 1.39 (d, J=6.4 Hz, 6H). (ESI⁺) m/z: 267.9 (M+H)⁺, (C₅H₇Br₂N₃).

B. 5-Bromo-1-isopropyl-3-phenyl-1H-1,2,4-triazole: To a solution of 3,5-dibromo-1-isopropyl-1,2,4-triazole (5.00 g, 18.4 mmol, 1.00 eq) in MeCN (60.0 mL) and H₂O (20.0 mL) was added phenylboronic acid (1.80 g, 14.7 mmol, 0.80 eq), K₂CO₃ (6.37 g, 46.1 mmol, 2.50 eq) and Pd(dppf)Cl₂ (1.35 g, 1.84 mmol, 0.10 eq) under N₂. The mixture was stirred at 25° C. for 2 h under N₂. After the reaction was completed, the reaction mixture was filtered and the filtrate was concentrated in vacuum to get a residue. The residue was purified by column chromatography (SiO₂, Petroleum ether:Ethyl acetate=100:1 to 3:1,R_{f}=0.50 (Petroleum ether:Ethyl acetate=3:1)) and by preparative-HPLC (using a Phenomenex Luna C18 (250 mm×70 mm×10 μm) and gradient of 12-42% acetonitrile in water containing 0.5% TFA over 5 min at a flow rate of 25.0 mL/min) to give the title compound (80.0 mg, 300 μmol, 1.60% yield, 99.8% purity in HPLC at 220 nm) as a yellow solid. ¹H NMR: (400 MHz, DMSO-d₆) δ 8.08 (d, J=6.8 Hz, 2H), 7.46-7.37 (m, 3H), 4.76-4.68 (m, 1H), 1.56 (d, J=6.4 Hz, 6H). (ESI⁺) m/z: 266.0 (M+H)⁺, (C₁₁H₁₂BrN₃).

C. 3-(5-(1-Isopropyl-3-phenyl-1H-1,2,4-triazol-5-yl)-1-oxoisoindolin-2-yl)piperidine-2,6-dione: To a solution of 5-bromo-1-isopropyl-3-phenyl-1,2,4-triazole (60.0 mg, 225 μmol, 1.00 eq) in dioxane (2.00 mL) and H₂O (0.10 mL) was added 3-[1-oxo-5-(4,4,5,5-tetramethyl-1,3,2-dioxaborolan-2-yl)isoindolin-2-yl]piperidine-2,6-dione (208 mg, 562 μmol, 2.50 eq), Ru-Phos-Pd-G₃ (18.8 mg, 22.5 μmol, 0.10 eq) and K₃PO₄ (95.5 mg, 449 μmol, 2.00 eq). The mixture was stirred at 100° C. for 2 h under N₂. After the reaction was completed, the reaction mixture was filtered and the filtrate was concentrated in vacuum to give a residue. The residue was purified by preparative-TLC (Dichloromethane:Methanol=10:1, R_{f}=0.50) and by preparative-HPLC (using a Phenomenex Luna C18 (150 mm×25 mm×5 μm) and gradient of 35-55% acetonitrile in water containing 0.5% TFA over 11 min at a flow rate of 25.0 mL/min) to give the title compound (56.9 mg, 132 μmol, 58.9% yield, 100% purity in HPLC at 220 nm) as a white solid. ¹H NMR: (400 MHz, DMSO-d₆) δ 11.0 (s, 1H), 8.07 (dd, J=8.0, 1.2 Hz, 2H), 7.94 (t, J=8.0 Hz, 2H), 7.84 (d, J=8.0 Hz, 1H), 7.52-7.42 (m, 3H), 5.18 (dd, J=13.2, 5.2 Hz, 1H), 4.79-4.73 (m, 1H), 4.54 (dd, J=50.8, 17.6 Hz, 2H), 2.94-2.90 (m, 1H), 2.63 (d, J=17.2 Hz, 1H), 2.44 (dd, J=13.2, 4.4 Hz, 1H), 2.08-2.04 (m, 1H). 1.50 (dd, J=6.4, 2.4 Hz, 6H). (ESI⁺) m/z: 430.1 (M+H)⁺, (C₂₄H₂₃N₅O₃).

### Example 611

### Synthesis of 3-(5-(5-(azepan-1-yl)-1-methyl-1H-1,2,4-triazol-3-yl)-1-oxoisoindolin-2-yl)piperidine-2,6-dione

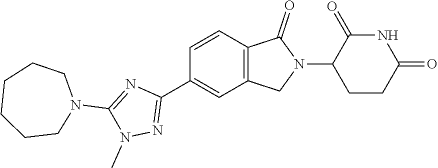 [SMILES: Cn1nc(-c2ccc3c(c2)CN(C2CCC(=O)NC2=O)C3=O)nc1N1CCCCCC1]

A. 1-(3-Bromo-1-methyl-1H-1,2,4-triazol-5-yl)azepane: To a solution of 3,5-dibromo-1-methyl-1H-1,2,4-triazole (0.30 g, 1.25 mmol, 1.00 eq) and azepane (123 mg, 1.25 mmol, 1.00 eq) in NMP (6.00 mL) was added K₂CO₃ (344 mg, 2.49 mmol, 2.00 eq) under N₂. The reaction mixture was stirred at 145° C. for 0.75 h under N₂. The reaction mixture was poured into H₂O (15.0 mL), extracted with ethyl acetate (3×20.0 mL), dried over Na₂SO₄ and concentrated in vacuum to give residue. The residue was purified by column chromatography (SiO₂, Petroleum ether:Ethyl acetate=100:1 to 30:1; TLC, Petroleum ether:Ethyl acetate=5:1, R_{f}=0.20) to give the title compound (0.60 g, 2.32 mmol, 92.9% yield) as yellow oil. ¹H NMR: (400 MHz, CDCl₃) δ 3.70 (s, 3H), 3.46-3.43 (m, 4H), 1.81-1.79 (m, 4H), 1.66-1.64 (m, 4H). (ESI⁺) m/z: 259.0 (M+H)⁺, (C₉H₁₅BrN₄).

B. 3-(5-(5-(Azepan-1-yl)-1-methyl-1H-1,2,4-triazol-3-yl)-1-oxoisoindolin-2-yl)piperidine-2,6-dione: To a solution of 1-(3-bromo-1-methyl-1H-1,2,4-triazol-5-yl)azepane (0.54 g, 2.08 mmol, 1.00 eq) and 3-(1-oxo-5-(4,4,5,5-tetramethyl-1,3,2-dioxaborolan-2-yl)isoindolin-2-yl)piperidine-2,6-dione (1.93 g, 5.21 mmol, 2.50 eq) in dioxane (6.00 mL) and H₂O (0.03 mL) added Ru-Phos-Pd-G₃ (174 mg, 208 μmol, 0.10 eq) and K₃PO₄ (884 mg, 4.17 mmol, 2.00 eq) under N₂. The reaction mixture was stirred at 100° C. under N₂ for 2 h. The reaction mixture was concentrated in vacuum to give a residue. The residue was purified by column chromatography (SiO₂, Dichloromethane:Methanol=100:1 to 80:1; TLC, Dichloromethane:Methanol=20:1, R_{f}=0.30) and preparative-HPLC (using a Phenomenex luna C18 (150 mm×25 mm×10 μm) and gradient of 17-47% acetonitrile in water (FA) over 10 min at a flow rate of 25 mL/min to give the title compound (306 mg, 725 μmol, 34.8% yield, 98.6% purity in HPLC at 220 nm) as white solid. ¹H NMR: (400 MHz, DMSO-d₆) δ 11.0 (s, 1H), 8.09 (s, 1H), 8.04 (d, J=8.0 Hz, 1H), 7.76 (d, J=8.0 Hz, 1H), 5.14-5.10 (m, 1H), 4.52-4.35 (m, 2H), 3.77 (s, 3H), 3.52-3.49 (m, 4H), 2.92-2.89 (m, 1H), 2.63-3.55 (m, 1H), 2.41-2.37 (m, 1H), 2.03-1.87 (m, 1H), 1.78-1.65 (m, 4H), 1.60-1.58 (m, 4H). (ESI⁺) m/z: 423.1 (M+H)⁺, (C₂₃H₂₇N₅O₃).

### Example 612

### Synthesis of 3-(5-(5-(4,4-dimethylpiperidin-1-yl)-1-methyl-1H-1,2,4-triazol-3-yl)-1-oxoisoindolin-2-yl)piperidine-2,6-dione

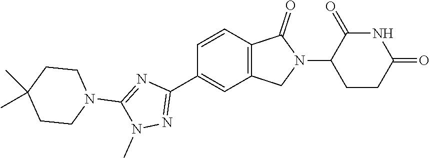 [SMILES: Cn1nc(-c2ccc3c(c2)CN(C2CCC(=O)NC2=O)C3=O)nc1N1CCC(C)(C)CC1]

A. 1-(3-Bromo-1-methyl-1H-1,2,4-triazol-5-yl)-4,4-dimethylpiperidine: A mixture of 3,5-dibromo-1-methyl-1,2,4-triazole (500 mg, 2.08 mmol, 1.00 eq), 4,4-dimethylpiperidine (234 mg, 2.08 mmol, 1.00 eq), K₂CO₃ (573 mg, 4.15 mmol, 2.00 eq) were taken up into a microwave tube in NMP (10.0 mL). The sealed tube was heated at 145° C. for 1 h under microwave. The reaction mixture was extracted with EA 60.0 mL (3×20.0 mL). The combined organic layers were washed with brine 150 mL (3×50.0 mL), dried over Na₂SO₄, filtered and concentrated under reduced pressure to give a residue. The filtrate was purified by preparative-HPLC (using a Phenomenex luna C18 (150 mm×25 mm×10 μm) and gradient of 43%-73% acetonitrile in water containing 0.05% FA over 15 min at a flow rate of 75 mL/min) to give the title compound (477 mg, 1.75 mmol, 84.1% yield, 100% purity in LCMS at 220 nm) as a white solid. ¹H NMR: (400 MHz, CDCl₃) δ 3.63 (s, 3H), 3.13-3.10 (m, 4H), 1.49-1.46 (m, 4H), 0.98 (s, 6H). (ESI⁺) m/z: 274.9 (M+3H)³⁺, (C₁₀H₁₇BrN₄).

B. 3-(5-(5-(4,4-Dimethylpiperidin-1-yl)-1-methyl-1H-1,2,4-triazol-3-yl)-1-oxoisoindolin-2-yl)piperidine-2,6-dione: A mixture of 1-(5-bromo-2-methyl-1,2,4-triazol-3-yl)-4,4-dimethyl-piperidine (477 mg, 1.75 mmol, 1.00 eq), 3-[1-oxo-5-(4,4,5,5-tetramethyl-1,3,2-dioxaborolan-2-yl)isoindolin-2-yl]piperidine-2,6-dione (1.62 g, 4.37 mmol, 2.50 eq), Ru-Phos-Pd-G₃ (146 mg, 174 μmol, 0.10 eq), K₃PO₄ (741 mg, 3.49 mmol, 2.00 eq) in dioxane (5.00 mL), H₂O (0.25 mL) was degassed and purged with N₂ for 3 times, and then the mixture was stirred at 100° C. for 0.5 h under N₂ atmosphere. The reaction mixture was extracted with EA 15.0 mL (3×5.00 mL). The combined organic layers were washed with brine 20.0 mL (2×10.0 mL), dried over Na₂SO₄, filtered and concentrated under reduced pressure to give a residue. The filtrate was purified by preparative-HPLC (using a Phenomenex luna C18 (150 mm×25 mm×10 μm) and gradient of 33%-63% acetonitrile in water containing 0.05% FA over 15 min at a flow rate of 75 mL/min) to get title compound (162 mg, 365 μmol, 20.9% yield, 98.4% purity in HPLC at 220 nm) as white solid. ¹H NMR: (400 MHz, DMSO-d₆) δ 11.0-10.9 (m, 1H), 8.12 (s, 1H), 8.07-8.05 (m, 1H), 7.77-7.75 (m, 1H), 5.16-5.09 (m, 1H), 4.52-4.35 (m, 2H), 3.71 (s, 3H), 3.18-3.16 (m, 4H), 2.92-2.91 (m, 1H), 2.62-2.58 (m, 1H), 2.45-2.35 (m, 1H), 2.04-2.00 (m, 1H), 1.49-1.47 (m, 4H), 0.99 (s, 6H). (ESI⁺) m/z: 437.0 (M+H)⁺, (C₂₃H₂₈N₆O₃).

### Example 613

### Synthesis of 3-(5-(5-(4,4-difluoropiperidin-1-yl)-1-methyl-1H-1,2,4-triazol-3-yl)-1-oxoisoindolin-2-yl)piperidine-2,6-dione

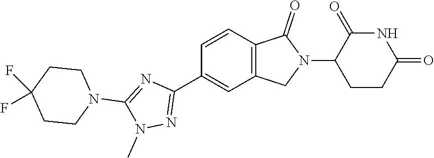 [SMILES: Cn1nc(-c2ccc3c(c2)CN(C2CCC(=O)NC2=O)C3=O)nc1N1CCC(F)(F)CC1]

A. 1-(3-Bromo-1-methyl-1H-1,2,4-triazol-5-yl)-4,4-difluoropiperidine: A mixture of 3,5-dibromo-1-methyl-1,2,4-triazole (500 mg, 2.08 mmol, 1.00 eq), 4,4-difluoropiperidine (251 mg, 2.08 mmol, 1.00 eq), K₂CO₃ (573 mg, 4.15 mmol, 2.00 eq) were taken up into a microwave tube in NMP (10.0 mL). The sealed tube was heated at 145° C. for 1 h under microwave. The reaction mixture was extracted with EA 45.0 mL (3×15.0 mL). The combined organic layers were washed with brine 60.0 mL (3×20.0 mL), dried over Na₂SO₄, filtered and concentrated under reduced pressure to give a residue. The filtrate was purified by preparative-HPLC (using a Phenomenex luna C18 (150 mm×25 mm×10 μm) and gradient of 25%-55% acetonitrile in water containing 0.05% FA over 15 min at a flow rate of 75 mL/min) to give the title compound (316 mg, 1.12 mmol, 54.1% yield, 100% purity in LCMS at 220 nm) as a white solid. ¹H NMR: (400 MHz, CDCl₃) δ 3.69 (s, 3H), 3.35-3.32 (m, 4H), 2.19-2.09 (m, 4H). (ESI⁺) m/z: 280.9 (M+H)⁺, (C₈H₁₁BrN₄F₂).

B. 3-(5-(5-(4,4-Difluoropiperidin-1-yl)-1-methyl-1H-1,2,4-triazol-3-yl)-1-oxoisoindolin-2-yl)piperidine-2,6-dione: A mixture of 1-(5-bromo-2-methyl-1,2,4-triazol-3-yl)-4,4-difluoro-piperidine (316 mg, 1.12 mmol, 1.00 eq), 3-[1-oxo-5-(4,4,5,5-tetramethyl-1,3,2-dioxaborolan-2-yl)isoindolin-2-yl]piperidine-2,6-dione (1.04 g, 2.81 mmol, 2.50 eq), Ru-Phos-Pd-G₃ (94.0 mg, 112 μmol, 0.10 eq), K₃PO₄ (477 mg, 2.25 mmol, 2.00 eq) in dioxane (3.00 mL), H₂O (0.15 mL) was degassed and purged with N₂ for 3 times, and then the mixture was stirred at 100° C. for 0.5 h under N₂ atmosphere. The reaction mixture was extracted with EA 15.0 mL (3×5.00 mL). The combined organic layers were washed with brine 20.0 mL (2×10.0 mL), dried over Na₂SO₄, filtered and concentrated under reduced pressure to give a residue. The filtrate was purified by preparative-HPLC (using a Phenomenex luna C18 (150 mm×25 mm×10 μm) and gradient of 21%-51% acetonitrile in water containing 0.05% FA over 15 min at a flow rate of 75 mL/min) to get title compound (108 mg, 243 μmol, 21.6% yield, 99.8% purity in HPLC at 220 nm) as white solid. ¹H NMR: (400 MHz, DMSO-d₆) δ 11.0-10.9 (m, 1H), 8.13-8.11 (m, 1H), 8.08-8.06 (m, 1H), 7.78-7.76 (m, 1H), 5.17-5.10 (m, 1H), 4.53-4.36 (m, 2H), 3.77 (s, 3H), 3.36-3.33 (m, 4H), 2.96-2.87 (m, 1H), 2.66-2.58 (m, 1H), 2.45-2.37 (m, 1H), 2.22-2.13 (m, 4H), 2.04-2.00 (m, 1H). (ESI⁺) m/z: 445.0 (M+H)⁺, (C₂₁H₂₂N₆O₃F₂).

### Example 614

### Synthesis of 3-(5-(5-cyclohexyl-1-methyl-1H-1,2,4-triazol-3-yl)-1-oxoisoindolin-2-yl)piperidine-2,6-dione

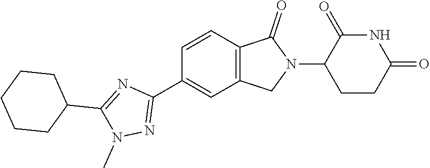 [SMILES: Cn1nc(-c2ccc3c(c2)CN(C2CCC(=O)NC2=O)C3=O)nc1C1CCCCC1]

A. 3-Bromo-5-(cyclohex-1-en-1-yl)-1-methyl-1H-1,2,4-triazole: To a solution of 3,5-dibromo-1-methyl-1,2,4-triazole (500 mg, 2.08 mmol, 1.00 eq) in MeOH (5.00 mL) was added 2-(cyclohexen-1-yl)-4,4,5,5-tetramethyl-1,3,2-dioxaborolane (475 mg, 2.28 mmol, 490 μL, 1.10 eq), (PPh₃)₂PdCl₂ (145 mg, 207 μmol, 0.10 eq) and CsF (630 mg, 4.15 mmol, 153 μL, 2.00 eq) under N₂. The mixture was stirred in a microwave reactor at 110° C. for 0.5 h under N₂. After the reaction was completed, the reaction mixture was filtered and the filtrate was concentrated in vacuum to get a residue. The residue was purified by preparative-TLC (Petroleum ether:Ethyl acetate=3:1, R_{f}=0.40) to give the title compound (300 mg, 1.02 mmol, 59.7% yield, 82.2% purity in LCMS at 220 nm) as a white solid. ¹H NMR: (400 MHz, CDCl₃) δ 6.21-6.18 (m, 1H), 3.87 (s, 3H), 2.42-2.40 (m, 2H), 2.28-2.25 (m, 2H), 1.77-1.75 (m, 2H), 1.71-1.68 (m, 2H). (ESI⁺) m/z: 242.0 (M+H)⁺, (C₉H₁₂BrN₃).

B. 3-Bromo-5-cyclohexyl-1-methyl-1H-1,2,4-triazole: To a solution of PtO₂ (23.1 mg, 101 μmol, 0.10 eq) and 3-bromo-5-(cyclohexen-1-yl)-1-methyl-1,2,4-triazole (300 mg, 1.02 mmol, 1.00 eq) in EtOH (10.0 mL) under N₂. Then the reaction mixture was degassed purged with H₂ for 3 times. Then the mixture was stirred at 25° C. for 3 h under H₂ (15 psi). After the reaction was completed, the reaction mixture was filtered through diatomite and the filtrate was concentrated in vacuum to give the title compound (200 mg, 790 μmol, 77.6% yield, 99.3% purity in LCMS at 220 nm) as a yellow solid. (ESI⁺) m/z: 244.0 (M+H)⁺, (C₉H₁₄BrN₃).

C. 3-(5-(5-Cyclohexyl-1-methyl-1H-1,2,4-triazol-3-yl)-1-oxoisoindolin-2-yl)piperidine-2,6-dione: To a solution of 3-bromo-5-cyclohexyl-1-methyl-1,2,4-triazole (200 mg, 790 μmol, 1.00 eq) in dioxane (2.00 mL) and H₂O (0.10 mL) was added 3-[1-oxo-5-(4,4,5,5-tetramethyl-1,3,2-dioxaborolan-2-yl)isoindolin-2-yl]piperidine-2,6-dione (731 mg, 1.98 mmol, 2.50 eq), Ru-Phos-Pd-G₃ (66.1 mg, 79.0 μmol, 0.10 eq) and K₃PO₄ (335 mg, 1.58 mmol, 2.00 eq) under N₂. The mixture was stirred at 100° C. for 2 h under N₂. After the reaction was completed, the reaction mixture was filtered and the filtrate was concentrated in vacuum to give a residue. The residue was purified by preparative-TLC (Dichloromethane:Methanol=10:1, R_{f}=0.50) and by preparative-HPLC (using a Phenomenex Luna C18 (150 mm×25 mm×5 μm) and gradient of 30-50% acetonitrile in water containing 0.5% TFA over 2 min at a flow rate of 25.0 mL/min) to give the title compound (151 mg, 360 μmol, 45.6% yield, 97.3% purity in HPLC at 220 nm) as a white solid. ¹H NMR: (400 MHz, DMSO-d₆) δ 11.0 (s, 1H), 8.17 (s, 1H), 8.11 (dd, J=8.0, 1.2 Hz, 1H), 7.78 (d, J=8.0 Hz, 1H), 5.13 (dd, J=13.2, 5.2 Hz, 1H), 4.45 (dd, J=49.6, 17.6 Hz, 2H), 3.88 (s, 3H), 2.98-2.88 (m, 2H), 2.64-2.58 (m, 1H), 2.40 (dd, J=13.2, 4.4 Hz, 1H), 2.05-2.00 (m, 1H), 1.91-1.79 (m, 4H), 1.71 (d, J=12.0 Hz, 1H), 1.62-1.36 (m, 4H), 1.33-1.23 (m, 1H). (ESI⁺) m/z: 408.2 (M+H)⁺, (C₂₂H₂₅N₅O₃).

### Example 615

### Synthesis of 3-(5-(1,5-dimethyl-2-(tetrahydro-2H-pyran-4-yl)-1H-imidazol-4-yl)-1-oxoisoindolin-2-yl)piperidine-2,6-dione

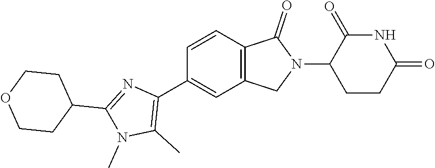 [SMILES: Cc1c(-c2ccc3c(c2)CN(C2CCC(=O)NC2=O)C3=O)nc(C2CCOCC2)n1C]

A. 1,5-Dimethyl-2-(tetrahydro-2H-pyran-4-yl)-1H-imidazole: To a solution of 5-bromo-1-methyl-2-tetrahydropyran-4-yl-imidazole (1.00 g, 4.08 mmol, 1.00 eq) and 2,4,6-trimethyl-1,3,5,2,4,6-trioxatriborinane (3.28 g, 13.0 mmol, 3.65 mL, 3.20 eq) in dioxane (30.0 mL) was added K₃PO₄ (1.73 g, 8.16 mmol, 2.00 eq) and Ru-Phos-Pd-G₃ (341 mg, 407 μmol, 0.10 eq) under N₂. The reaction mixture was stirred at 70° C. for 10 h under N₂. The reaction mixture was concentrated in vacuum to give residue. The residue was purified by column chromatography (SiO₂, Dichloromethane:Methanol=100:1 to 50:1; TLC, Dichloromethane:Methanol=10:1, R_{f}=0.30) to give the title compound (0.55 g, 2.68 mmol, 65.5% yield) as colorless oil. ¹H NMR: (400 MHz, CDCl₃) δ 6.70 (s, 1H), 4.10-4.07 (m, 2H), 3.56-3.50 (m, 2H), 3.46 (s, 3H), 2.89-2.83 (m, 1H), 2.18 (s, 3H), 2.07-2.01 (m, 2H), 1.99-1.77 (m, 2H). (ESI⁺) m/z: 181.3 (M+H)⁺, (C₁₀H₁₆N₂O).

B. 4-Bromo-1,5-dimethyl-2-(tetrahydro-2H-pyran-4-yl)-1H-imidazole: To a solution of 1,5-dimethyl-2-tetrahydropyran-4-yl-imidazole (0.50 g, 2.77 mmol, 1.00 eq) in DMF (5.00 mL) was added a solution of NBS (493 mg, 2.77 mmol, 1.00 eq) in DMF (5.00 mL) at 0° C. The reaction mixture was stirred at 25° C. for 2 h. The reaction mixture was poured into H₂O (15.0 mL), extracted with ethyl acetate (3×20.0 mL), dried over Na₂SO₄ and concentrated in vacuum to give residue. The residue was purified by preparative-HPLC (using a Welch Xtimate C18 (200 mm×40 mm×10 μm) and gradient of 1-15% acetonitrile in water containing 0.05% TFA over 12 min at a flow rate of 25.0 mL/min) to give the title compound (0.32 g, 1.23 mmol, 44.5% yield) as white solid. m/z: 260.2 (M+H)⁺, (C₁₀H₁₅BrN₂O).

C. 3-(5-(1,5-Dimethyl-2-(tetrahydro-2H-pyran-4-yl)-1H-imidazol-4-yl)-1-oxoisoindolin-2-yl)piperidine-2,6-dione: To a solution of 4-bromo-1,5-dimethyl-2-tetrahydropyran-4-yl-imidazole (0.30 g, 1.16 mmol, 1.00 eq), 3-[1-oxo-5-(4,4,5,5-tetramethyl-1,3,2-dioxaborolan-2-yl)isoindolin-2-yl]piperidine-2,6-dione (1.07 g, 2.89 mmol, 2.50 eq) in dioxane (5.00 mL) and H₂O (0.25 mL) was added K₃PO₄ (491 mg, 2.32 mmol, 2.00 eq), Ru-Phos-Pd-G₃ (96.8 mg, 115 μmol, 0.10 eq) under N₂. The reaction mixture was stirred at 100° C. for 2 h under N₂. The reaction mixture was concentrated in vacuum to give residue. The residue was purified by column chromatography (SiO₂, Dichloromethane:Methanol=100:1 to 50:1; TLC, Dichloromethane:Methanol=10:1, R_{f}=0.30) and preparative-HPLC (using a Welch Xtimate C18 (150 mm×25 mm×5 μm) and gradient of 0-30% acetonitrile in water containing 0.05% TFA over 11 min at a flow rate of 25.0 mL/min) to give the title compound (247 mg, 561 μmol, 48.4% yield, 95.6% purity in HPLC at 220 nm) as white solid. ¹H NMR: (400 MHz, DMSO-d₆) δ 11.0 (s, 1H), 7.91-7.89 (m, 1H), 7.82 (s, 1H), 7.82 (d, J=7.6 Hz, 1H), 5.17-5.13 (m, 1H), 4.56-4.39 (m, 2H), 4.00-3.97 (m, 2H), 3.76 (s, 3H), 3.53-3.46 (m, 2H), 3.12-2.80 (m, 1H), 2.64-2.50 (m, 1H), 2.48-2.45 (m, 1H), 2.41 (s, 3H), 1.93-1.90 (m, 1H), 1.87-1.83 (m, 5H). (ESI⁺) m/z: 423.0 (M+H)⁺, (C₂₃H₂₆N₄O₄).

### Example 616

### Synthesis of 3-(5-(1-methyl-5-((1r,4r)-4-methylcyclohexyl)-1H-1,2,4-triazol-3-yl)-1-oxoisoindolin-2-yl)piperidine-2,6-dione

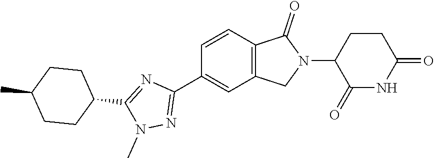 [SMILES: Cn1nc(-c2ccc3c(c2)CN(C2CCC(=O)NC2=O)C3=O)nc1[C@H]1CC[C@H](C)CC1]

A. 3-Bromo-1-methyl-5-(4-methylcyclohex-1-en-1-yl)-1H-1,2,4-triazole: A solution of 3,5-dibromo-1-methyl-1,2,4-triazole (0.30 g, 1.25 mmol, 1.00 eq), 4,4,5,5-tetramethyl-2-(4-methylcyclohexen-1-yl)-1,3,2-dioxaborolane (304 mg, 1.37 mmol, 1.10 eq), (PPh₃)₂PdCl₂ (87.4 mg, 124 μmol, 0.10 eq) and CsF (378 mg, 2.49 mmol, 2.00 eq) in MeOH (3.00 mL) was stirred at 110° C. under N₂ in MW for 0.75 h. The reaction mixture was concentrated in vacuum to give residue. The residue was purified by preparative-HPLC (using a Welch Xtimate XB-CN (250 mm×70 mm×10 μm) and gradient of 1-16% EtOH in Hexane containing 0.05% TFA over 30 min at a flow rate of 25.0 mL/min) to give the title compound (1.16 g, 4.53 mmol, 72.7% yield) as yellow oil. ¹H NMR: (400 MHz, CDCl₃) (6.17-6.16 (m, 1H), 3.87 (s, 3H), 2.51-2.49 (m, 1H), 2.40-2.35 (m, 2H), 1.88-1.86 (m, 2H), 1.85-1.83 (m, 1H), 1.38-1.35 (m, 1H), 1.02 (d, J=6.4 Hz, 3H). (ESI⁺) m/z: 257.0 (M+H)⁺, (C₁₀H₁₄BrN₃).

B. 3-Bromo-1-methyl-5-(4-methylcyclohexyl)-1H-1,2,4-triazole: To a solution of 3-bromo-1-methyl-5-(4-methylcyclohex-1-en-1-yl)-1H-1,2,4-triazole (0.30 g, 1.17 mmol, 1.00 eq) in EtOH (10.0 mL) was added PtO₂ (26.6 mg, 117 μmol, 0.10 eq). The reaction mixture was degassed and purged with N₂ for 3 times, then degassed and purged with H₂ for 3 times. The reaction mixture was warmed to 25° C. and stirred at 50 psi under H₂ for 12 h. The reaction mixture was filtered. The filtrate was concentrated in vacuum to give residue. The residue was purified by preparative-TLC (SiO₂, Petroleum ether:Ethyl acetate=3:1; TLC, Petroleum ether:Ethyl acetate=3:1, R_{f}=0.45) to give the title compound (100 mg, 368 μmol, 31.4% yield, 95.1% purity in HPLC at 220 nm) as colorless oil. m/z: 260.2 (M+H)⁺, (C₁₀H₁₆BrN₃).

C. 3-Bromo-1-methyl-5-((1r,4r)-4-methylcyclohexyl)-1H-1,2,4-triazole: The 3-bromo-1-methyl-5-(4-methylcyclohexyl)-1,2,4-triazole (0.10 g, 387 μmol, 1.00 eq) was purified by Chiral SFC (using a Welch Xtimate C18 (250 mm×30 mm×10 μm) and gradient of 20% i-PrOH containing 0.1% NH₃·H₂O over 13 min at a flow rate of 25.0 mL/min)) to give the title compound (40.0 mg, 154 μmol, 40.0% yield) as colorless oil. m/z: 259.8 (M+H)⁺, (C₁₀H₁₆BrN₃).

D. 3-(5-(1-Methyl-5-((1r,4r)-4-methylcyclohexyl)-1H-1,2,4-triazol-3-yl)-1-oxoisoindolin-2-yl)piperidine-2,6-dione: To a solution of 3-bromo-1-methyl-5-((1r,4r)-4-methylcyclohexyl)-1H-1,2,4-triazole (40.0 mg, 154 μmol, 1.00 eq), 3-[1-oxo-5-(4,4,5,5-tetramethyl-1,3,2-dioxaborolan-2-yl)isoindolin-2-yl]piperidine-2,6-dione (114 mg, 309 μmol, 2.00 eq) in dioxane (1.00 mL) and H₂O (0.05 mL) was added Ru-Phos-Pd-G₃ (12.9 mg, 15.4 μmol, 0.10 eq), K₃PO₄ (65.7 mg, 309 μmol, 2.00 eq) under N₂. The reaction mixture was stirred at 100° C. for 2 h under N₂. The reaction mixture was concentrated in vacuum to give residue. The residue was purified by preparative-TLC (SiO₂, Dichloromethane:Methanol=20:1; TLC, Dichloromethane:Methanol=20:1, R_{f}=0.30) and preparative-HPLC (using a Welch Xtimate C18 (150 mm×25 mm×5 μm) and gradient of 26-56% acetonitrile in water containing 0.05% TFA over 13 min at a flow rate of 25.0 mL/min) to give the title compound (22.6 mg, 53.5 μmol, 34.5% yield, 99.8% purity in HPLC at 220 nm) as white solid. ¹H NMR: (400 MHz, DMSO-d₆) δ 11.0 (s, 1H), 8.18 (s, 1H), 8.13-8.11 (m, 1H), 7.79 (d, J=8.0 Hz, 1H), 5.15-5.10 (m, 1H), 4.55-4.37 (m, 2H), 3.87 (s, 3H), 3.07-3.06 (m, 1H), 2.95-2.75 (m, 1H), 2.63-2.58 (m, 1H), 2.48-2.45 (m, 1H), 2.04-1.86 (m, 1H), 1.84-1.58 (m, 9H), 1.00 (d, J=7.2 Hz, 3H). (ESI⁺) m/z: 422.2 (M+H)⁺, (C₂₃H₂₇N₅O₃).

### Example 617

### Synthesis of 3-(5-(1-methyl-5-((1s,4s)-4-methylcyclohexyl)-1H-1,2,4-triazol-3-yl)-1-oxoisoindolin-2-yl)piperidine-2,6-dione

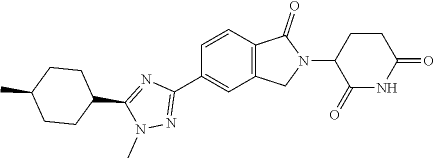

A. 3-Bromo-1-methyl-5-((1s,4s)-4-methylcyclohexyl)-1H-1,2,4-triazole: The 3-bromo-1-methyl-5-(4-methylcyclohexyl)-1,2,4-triazole (0.10 g, 387 μmol, 1.00 eq) was purified by Chiral SFC (using a Welch Xtimate C18 (250 mm×30 mm×10 μm) and gradient of 20% i-PrOH containing 0.1% NH₃·H₂O over 13 min at a flow rate of 25.0 mL/min)) to give the title compound (40.0 mg, 154 μmol, 40.0% yield) as colorless oil. m/z: 259.8 (M+H)⁺, (C₁₀H₁₆BrN₃).

B. 3-(5-(1-Methyl-5-((1s,4s)-4-methylcyclohexyl)-1H-1,2,4-triazol-3-yl)-1-oxoisoindolin-2-yl)piperidine-2,6-dione: To a solution of 3-bromo-1-methyl-5-((1s,4s)-4-methylcyclohexyl)-1H-1,2,4-triazole (40.0 mg, 154 μmol, 1.00 eq), 3-[1-oxo-5-(4,4,5,5-tetramethyl-1,3,2-dioxaborolan-2-yl)isoindolin-2-yl]piperidine-2,6-dione (114 mg, 309 μmol, 2.00 eq) in dioxane (1.00 mL) and H₂O (0.05 mL) was added Ru-Phos-Pd-G₃ (12.9 mg, 15.4 μmol, 0.10 eq), K₃PO₄ (65.7 mg, 309 μmol, 2.00 eq) under N₂. The reaction mixture was stirred at 100° C. for 2 h under N₂. The reaction mixture was concentrated in vacuum to give residue. The residue was purified by preparative-TLC (SiO₂, Dichloromethane:Methanol=20:1; TLC, Dichloromethane:Methanol=20:1, R_{f}=0.30) and preparative-HPLC (using a Welch Xtimate C18 (150 mm×25 mm×5 μm) and gradient of 26-56% acetonitrile in water containing 0.05% TFA over 13 min at a flow rate of 25.0 mL/min) to give the title compound (9.00 mg, 20.5 μmol, 13.2% yield, 96.4% purity in HPLC at 220 nm) as white solid. ¹H NMR: (400 MHz, DMSO-d₆) δ 10.9 (s, 1H), 8.16 (s, 1H), 8.10-8.08 (m, 1H), 7.76 (d, J=8.0 Hz, 1H), 5.14-5.09 (m, 1H), 4.52-4.36 (m, 2H), 3.87 (s, 3H), 2.90-2.86 (m, 1H), 2.62-2.58 (m, 1H), 2.49-2.40 (m, 1H), 2.22-2.00 (m, 1H), 1.95-1.91 (m, 2H), 1.88-1.86 (m, 2H), 1.79-1.75 (m, 2H), 1.61-1.58 (m, 2H), 1.11-1.07 (m, 2H), 0.91 (d, J=6.4 Hz, 3H). (ESI⁺) m/z: 422.2 (M+H)⁺, (C₂₃H₂₇N₅O₃).

### Example 618

### Synthesis of 3-(5-(4-methyl-3-phenyl-1H-pyrazol-1-yl)-1-oxoisoindolin-2-yl)piperidine-2,6-dione

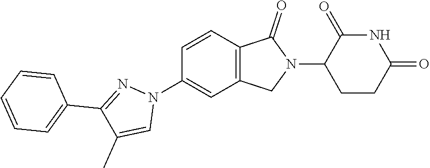 [SMILES: Cc1cn(-c2ccc3c(c2)CN(C2CCC(=O)NC2=O)C3=O)nc1-c1ccccc1]

A. 4-Methyl-3-phenyl-1H-pyrazole: To a solution of (E)-2-methyl-3-phenyl-prop-2-enal (1.00 g, 6.84 mmol, 1.00 eq) in H₂O (17.0 mL) was added 4-methylbenzenesulfonohydrazide (1.53 g, 8.21 mmol, 1.20 eq), NaOH (410 mg, 10.2 mmol, 1.50 eq) and TBAB (2.21 g, 6.84 mmol, 1.00 eq). The mixture was stirred at 80° C. for 15 h. The reaction mixture was extracted with EtOAc (3×20.0 mL) and the organic layer was washed with water (20.0 mL), brine (20.0 mL), dried over N₂SO₄ and filtered. The filtrate was concentrated in vacuum to get a residue. The residue was purified by column chromatography (SiO₂, Petroleum ether:Ethyl acetate=100:1 to 10:1, R_{f}=0.40 (Petroleum ether:Ethyl acetate=1:1)) to give the title compound (10.0 g, 63.2 mmol, 92.4% yield) as an off-white solid. ¹H NMR: (400 MHz, CDCl₃) δ 7.59 (d, J=7.2 Hz, 2H), 7.49-7.43 (m, 3H), 7.40-7.35 (m, 1H), 2.25 (s, 3H). (ESI⁺) m/z: 159.0 (M+H)⁺, (C₁₀H₁₀N₂).

B. 5-Amino-4-(5-(4-methyl-3-phenyl-1H-pyrazol-1-yl)-1-oxoisoindolin-2-yl)-5-oxopentanoic acid and 5-amino-4-(5-(4-methyl-5-phenyl-1H-pyrazol-1-yl)-1-oxoisoindolin-2-yl)-5-oxopentanoic acid: To a solution of 4-methyl-3-phenyl-1H-pyrazole (300 mg, 1.90 mmol, 1.00 eq) in DMF (14.0 mL) and H₂O (1.40 mL) was added tert-butyl 5-amino-4-(5-bromo-1-oxo-isoindolin-2-yl)-5-oxo-pentanoate (753 mg, 1.90 mmol, 1.00 eq), Cs₂CO₃ (2.47 g, 7.59 mmol, 4.00 eq), CuI (36.1 mg, 189 μmol, 0.10 eq) and 8-hydroxyquinoline (27.5 mg, 189 μmol, 32.7 μL, 0.10 eq). The mixture was stirred in a microwave reactor at 145° C. for 1 h. Then the reaction mixture was filtered and the filtrate was concentrated in vacuum to get a residue. The residue was purified by prep-HPLC (using a Phenomenex Luna C18 (200 mm×40 mm 10 μm) and gradient of 20-50% acetonitrile in water containing 0.5% HCl over 12 min at a flow rate of 25.0 mL/min) give the title compound 796-3 (100 mg, 238 μmol, 4.20% yield, 100% purity in LCMS at 220 nm) as a yellow solid ((ESI⁺) m/z: 419.0 (M+H)⁺, (C₂₃H₂₂N₄O₄)) and compound 834-1 (30.0 mg, 71.6 μmol, 1.26% yield, 100% purity in LCMS at 220 nm) as a yellow solid ((ESI⁺) m/z: 419.0 (M+H)⁺, (C₂₃H₂₂N₄O₄)).

C. 3-(5-(4-Methyl-3-phenyl-1H-pyrazol-1-yl)-1-oxoisoindolin-2-yl)piperidine-2,6-dione: To a solution of 5-amino-4-[5-(4-methyl-3-phenyl-pyrazol-1-yl)-1-oxo-isoindolin-2-yl]-5-oxo-pentanoic acid (100 mg, 238 μmol, 1.00 eq) in ACN (5.00 mL) was added CDI (116 mg, 716 μmol, 3.00 eq). The mixture was stirred at 100° C. for 2 h. Then the reaction mixture was filtered and the filtrate was concentrated in vacuum to get a residue. The residue was purified by prep-HPLC (using a Welch Xtimate C18 (150 mm×25 mm 5 μm) and gradient of 28-58% acetonitrile in water containing 0.5% HCl over 8 min at a flow rate of 25.0 mL/min) to give the title compound (29.4 mg, 72.3 μmol, 30.2% yield, 98.2% purity in HPLC at 220 nm) as a yellow solid. ¹H NMR: (400 MHz, DMSO-d₆) δ 11.0 (s, 1H), 8.53 (d, J=0.8 Hz, 1H), 8.12 (s, 1H), 8.02 (dd, J=8.4, 2.0 Hz, 1H), 7.83 (d, J=8.0 Hz, 1H), 7.81-7.77 (m, 2H), 7.53-7.47 (m, 2H), 7.43-7.38 (m, 1H), 5.13 (dd, J=13.2, 5.2 Hz, 1H), 4.47 (dd, J=51.2, 17.6 Hz, 2H), 2.97-2.87 (m, 1H), 2.65-2.58 (m, 1H), 2.47-2.37 (m, 1H), 2.29 (s, 3H), 2.06-1.99 (m, 1H). (ESI⁺) m/z: 400.9 (M+H)⁺, (C₂₃H₂₀N₄O₃).

### Example 619

### Synthesis of 3-(5-(1-methyl-5-(4-(trifluoromethyl)phenyl)-1H-1,2,4-triazol-3-yl)-1-oxoisoindolin-2-yl)piperidine-2,6-dione

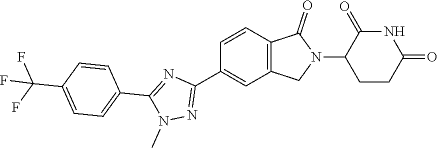 [SMILES: Cn1nc(-c2ccc3c(c2)CN(C2CCC(=O)NC2=O)C3=O)nc1-c1ccc(C(F)(F)F)cc1]

A. 3-Bromo-1-methyl-5-(4-(trifluoromethyl)phenyl)-1H-1,2,4-triazole: To a solution of 3,5-dibromo-1-methyl-1,2,4-triazole (500 mg, 2.08 mmol, 1.00 eq) in ACN (6.00 mL) and H₂O (2.00 mL) was added [4-(trifluoromethyl)phenyl]boronic acid (315 mg, 1.66 mmol, 0.80 eq), PdCl₂(dppf) (151 mg, 207 μmol, 0.10 eq) and K₂CO₃ (717 mg, 5.19 mmol, 2.50 eq) under N₂. The mixture was stirred at 25° C. for 2 h under N₂. After the reaction was completed, the reaction mixture was filtered and the filtrate was concentrated in vacuum to give a residue. The residue was purified by column chromatography (SiO₂, Petroleum ether: Ethyl acetate=100:1 to 5:1, R_{f}=0.40 (Petroleum ether:Ethyl acetate=5:1)) and by preparative-HPLC (using a Phenomenex Luna C18 (150 mm×25 mm×5 μm) and gradient of 38-68% acetonitrile in water containing 0.5% TFA over 2 min at a flow rate of 25.0 mL/min) to give the title compound (70.0 mg, 226 μmol, 15.4% yield, 99.2% purity in LCMS at 220 nm) as a white solid. ¹H NMR: (400 MHz, DMSO-d₆) δ 8.00 (dd, J=29.2, 8.4 Hz, 4H), 3.98 (s, 3H). (ESI⁺) m/z: 305.9 (M+H)⁺, (C₁₀H₇BrF₃N₃).

B. 3-(5-(1-Methyl-5-(4-(trifluoromethyl)phenyl)-1H-1,2,4-triazol-3-yl)-1-oxoisoindolin-2-yl)piperidine-2,6-dione: To a solution of 3-bromo-1-methyl-5-[4-(trifluoromethyl)phenyl]-1,2,4-triazole (60.0 mg, 194 μmol, 1.00 eq) in dioxane (2.00 mL) and H₂O (0.10 mL) was added 3-[1-oxo-5-(4,4,5,5-tetramethyl-1,3,2-dioxaborolan-2-yl)isoindolin-2-yl]piperidine-2,6-dione (179 mg, 486 μmol, 2.50 eq), Ru-Phos-Pd-G₃ (16.2 mg, 19.4 μmol, 0.10 eq) and K₃PO₄ (82.5 mg, 388 μmol, 2.00 eq) under N₂. The mixture was stirred at 100° C. for 2 h under N₂. After the reaction was completed, the reaction mixture was filtered and the filtrate was concentrated in vacuum to give a residue. The residue was purified by preparative-TLC (Dichloromethane:Methanol=10:1, R_{f}=0.40) and by preparative-HPLC (using a Phenomenex Luna C18 (150 mm×25 mm×5 μm) and gradient of 30-60% acetonitrile in water containing 0.5% TFA over 2 min at a flow rate of 25.0 mL/min) to give the title compound (41.3 mg, 88.1 μmol, 45.3% yield, 100% purity in HPLC at 220 nm) as a white solid. ¹H NMR: (400 MHz, DMSO-d₆) δ 11.0 (s, 1H), 8.29 (s, 1H), 8.21 (dd, J=8.0, 1.2 Hz, 1H), 8.12 (d, J=8.4 Hz, 2H), 7.97 (d, J=8.4 Hz, 2H), 7.84 (d, J=7.6 Hz, 1H), 5.14 (dd, J=13.2, 4.8 Hz, 1H), 4.49 (dd, J=50.8, 17.6 Hz, 2H), 4.09 (s, 3H), 2.98-2.88 (m, 1H), 2.62 (d, J=17.2 Hz, 1H), 2.41 (dd, J=12.8, 4.0 Hz, 1H), 2.07-2.01 (m, 1H). (ESI⁺) m/z: 470.1 (M+H)⁺, (C₂₃H₁₈F₃N₅O₃).

### Example 620

### Synthesis of 3-(5-(4-Methyl-3-phenyl-1H-pyrazol-1-yl)-1-oxoisoindolin-2-yl)piperidine-2,6-dione

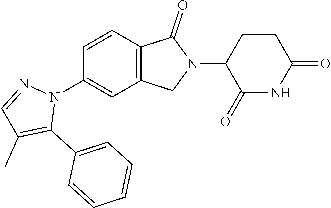 [SMILES: Cc1cnn(-c2ccc3c(c2)CN(C2CCC(=O)NC2=O)C3=O)c1-c1ccccc1]

A. 3-(5-(4-Methyl-3-phenyl-1H-pyrazol-1-yl)-1-oxoisoindolin-2-yl)piperidine-2,6-dione: To a solution of 5-amino-4-(5-(4-methyl-5-phenyl-1H-pyrazol-1-yl)-1-oxoisoindolin-2-yl)-5-oxopentanoic acid (30.0 mg, 71.6 μmol, 1.00 eq) in ACN (2.00 mL) was added CDI (34.8 mg, 215 μmol, 3.00 eq). The mixture was stirred at 100° C. for 2 h. Then the reaction mixture was filtered and the filtrate was concentrated in vacuum to get a residue. The residue was purified by prep-HPLC (using a Welch Xtimate C18 (150 mm×25 mm 5 μm) and gradient of 22-52% acetonitrile in water containing 0.5% HCl over 8 min at a flow rate of 25.0 mL/min) to give the title compound (2.72 mg, 6.47 μmol, 9.01% yield, 95.2% purity in HPLC at 220 nm) as an off-white solid. ¹H NMR: (400 MHz, DMSO-d₆) δ 10.9 (s, 1H), 7.72 (s, 1H), 7.62 (d, J=8.0 Hz, 1H), 7.53 (d, J=1.2 Hz, 1H), 7.45-7.38 (m, 3H), 7.25-7.21 (m, 2H), 7.20-7.16 (m, 1H), 5.08 (dd, J=13.2, 5.2 Hz, 1H), 4.34 (dd, J=54.0, 17.2 Hz, 2H), 2.95-2.85 (m, 1H), 2.62-2.56 (m, 1H), 2.43-2.34 (m, 1H), 2.06 (s, 3H), 2.03-1.96 (m, 1H). (ESI⁺) m/z: 401.0 (M+H)⁺, (C₂₃H₂₀N₄O₃).

### Example 621

### Synthesis of 3-(5-(1-cyclopropyl-5-phenyl-1H-pyrazol-3-yl)-1-oxoisoindolin-2-yl)piperidine-2,6-dione

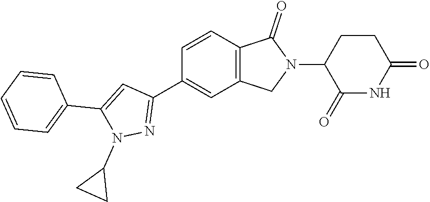 [SMILES: O=C1CCC(N2Cc3cc(-c4cc(-c5ccccc5)n(C5CC5)n4)ccc3C2=O)C(=O)N1]

A. 3-Iodo-5-phenyl-1H-pyrazole: To a solution of 5-phenyl-1H-pyrazol-3-amine (10.0 g, 62.8 mmol, 1.00 eq) in ACN (500 mL) and H₂O (100 mL) was added NaI (28.2 g, 188 mmol, 3.00 eq), TsOH (32.4 g, 188 mmol, 3.00 eq) and NaNO₂ (13.0 g, 188 mmol, 3.00 eq) at 0° C. under N₂. The mixture reaction was stirred at 0° C. for 30 mins. Then the mixture reaction was stirred at 25° C. for 12 h under N₂. After the reaction was completed, the reaction mixture was diluted with water (200 mL) and extracted with ethyl acetate (3×200 mL), dried over Na₂SO₄, filtered and the filtrate was concentrated in vacuum to get a residue. The residue was purified by column chromatography (SiO₂, Dichloromethane:Methanol=100:1 to 10:1, R_{f}=0.60 (Dichloromethane:Methanol=10:1)) to give the title compound (3.60 g, 11.2 mmol, 21.2% yield, 84.7% purity in LCMS at 220 nm) as yellow oil. ¹H NMR: (400 MHz, DMSO-d₆) δ 7.75-7.72 (m, 2H), 7.44 (t, J=8.0 Hz, 2H), 7.37-7.33 (m, 1H), 6.92 (s, 1H). (ESI⁺) m/z: 270.9 (M+H)⁺, (C₉H₇IN₂).

B. 1-cyclopropyl-3-iodo-5-phenyl-1H-pyrazole: To a solution of 3-iodo-5-phenyl-1H-pyrazole (1.00 g, 3.14 mmol, 1.00 eq) in DCE (10.0 mL) was added cyclopropylboronic acid (2.69 g, 31.3 mmol, 10.0 eq), Cu(OAc)₂ (569 mg, 3.14 mmol, 1.00 eq), Na₂CO₃ (997 mg, 9.41 mmol, 3.00 eq) and Bpy (1.47 g, 9.41 mmol, 3.00 eq) under N₂. The mixture was stirred at 70° C. for 10 h under N₂. After the reaction was completed, the reaction mixture was filtered and the filtrate was concentrated in vacuum to give a residue. The residue was purified by column chromatography (SiO₂, Petroleum ether:Ethyl acetate=100:1 to 5:1, R_{f}=0.55 (Petroleum ether:Ethyl acetate=5:1)) to give the title compound (140 mg, 334 μmol, 14.3% yield, 74.2% purity in LCMS at 220 nm) as yellow oil. ¹H NMR: (400 MHz, DMSO-d₆) δ 7.64-7.61 (m, 2H), 7.53-7.46 (m, 3H), 6.63 (d, J=4.8 Hz, 1H), 3.76-3.69 (m, 1H), 0.93 (d, J=4.0 Hz, 4H). (ESI⁺) m/z: 311.0 (M+H)⁺, (C₁₂H₁₁IN₂).

C. 3-(5-(1-cyclopropyl-5-phenyl-1H-pyrazol-3-yl)-1-oxoisoindolin-2-yl)piperidine-2,6-dione: To a solution of 1-cyclopropyl-3-iodo-5-phenyl-pyrazole (120 mg, 287 μmol, 1.00 eq) in dioxane (2.00 mL) and H₂O (0.10 mL) was added 3-[1-oxo-5-(4,4,5,5-tetramethyl-1,3,2-dioxaborolan-2-yl)isoindolin-2-yl]piperidine-2,6-dione (265 mg, 717 μmol, 2.50 eq), Ru-Phos-Pd-G₃ (24.0 mg, 28.7 μmol, 0.10 eq) and K₃PO₄ (121 mg, 574 μmol, 2.00 eq) under N₂. The mixture was stirred at 100° C. for 2 h under N₂. After the reaction was completed, the reaction mixture was filtered and the filtrate was concentrated in vacuum to give a residue. The residue was purified by preparative-TLC (Dichloromethane:Methanol=10:1, R_{f}=0.50) and by preparative-HPLC (using a Phenomenex Luna C18 (150 mm×25 mm×10 μm) and gradient of 35-65% acetonitrile in water containing 0.5% TFA over 2 min at a flow rate of 25.0 mL/min) to give the title compound (16.5 mg, 38.6 μmol, 13.4% yield, 100% purity in HPLC at 220 nm) as a pink solid. ¹H NMR: (400 MHz, DMSO-d₆) δ 11.0 (s, 1H), 8.06 (s, 1H), 8.00-7.97 (m, 1H), 7.77-7.72 (m, 3H), 7.57-7.52 (m, 2H), 7.49-7.45 (m, 1H), 7.04 (s, 1H), 5.13 (dd, J=8.8, 4.8 Hz, 1H), 4.45 (dd, J=52.4, 17.6 Hz, 2H), 3.87-3.79 (m, 1H), 2.97-2.87 (m, 1H), 2.64-2.58 (m, 1H), 2.44-2.39 (m, 1H), 2.05-2.01 (m, 1H), 1.09-0.98 (m, 4H). (ESI⁺) m/z: 427.1 (M+H)⁺, (C₂₅H₂₂N₄O₃).

### Example 622

### Synthesis of 3-(5-(5-(4-chlorophenyl)-1-methyl-1H-1,2,4-triazol-3-yl)-1-oxoisoindolin-2-yl)piperidine-2,6-dione

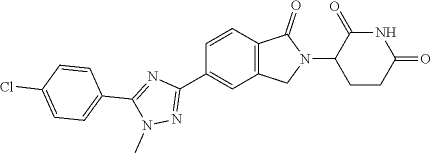 [SMILES: Cn1nc(-c2ccc3c(c2)CN(C2CCC(=O)NC2=O)C3=O)nc1-c1ccc(Cl)cc1]

A. 3-Bromo-5-(4-chlorophenyl)-1-methyl-1H-1,2,4-triazole: To a solution of 3,5-dibromo-1-methyl-1H-1,2,4-triazole (500 mg, 2.08 mmol, 1.00 eq)), K₂CO₃ (717 mg, 5.19 mmol, 2.50 eq) and (4-chlorophenyl)boronic acid (259 mg, 1.66 mmol, 0.80 eq) in ACN (6.00 mL) and H₂O (2.00 mL) was added Pd(dppf)Cl₂ (151 mg, 207 μmol, 0.10 eq) under N₂. The mixture was stirred at 40° C. for 12 h under N₂. The mixture was filtered to collect liquid and concentrated under reduced pressure to get residue. The residue was purified by preparative-HPLC (using a Phenomenex luna C18 (150 mm×25 mm 10 μm) and gradient of 35-65% acetonitrile in water containing 0.5% TFA over 40 min at a flow rate of 25 mL/min) to give the title compound (220 mg, 807 μmol, 38.8% yield, 100% purity in LCMS at 220 nm) as light yellow solid. ¹H NMR: (400 MHz, DMSO-d₆) δ 7.81-7.79 (d, J=8.0 Hz, 2H), 7.65-7.63 (d, J=8.0 Hz, 2H), 3.94 (s, 3H). (ESI⁺) m/z: 273.8 (M+H)⁺, (C₉H₇BrClN₃).

B. 3-(5-(5-(4-Chlorophenyl)-1-methyl-1H-1,2,4-triazol-3-yl)-1-oxoisoindolin-2-yl)piperidine-2,6-dione: To a solution of 3-bromo-5-(4-chlorophenyl)-1-methyl-1H-1,2,4-triazole (200 mg, 733 μmol, 1.00 eq), 3-(1-oxo-5-(4,4,5,5-tetramethyl-1,3,2-dioxaborolan-2-yl)isoindolin-2-yl)piperidine-2,6-dione (679 mg, 1.83 mmol, 2.50 eq) and K₃PO₄ (311 mg, 1.47 mmol, 2.00 eq) in dioxane (5.00 mL) and H₂O (0.25 mL) was added Ru-Phos-Pd-G₃ (122 mg, 146 μmol, 0.20 eq) under N₂. The mixture was stirred at 100° C. for 2 h under N₂. The mixture was filtered to collect liquid and concentrated under reduced pressure to get residue. The crude product was purified by preparative-HPLC (using a Phenomenex luna C18 (150 mm×25 mm 10 μm) and gradient of 28-48% acetonitrile in water containing 0.5% TFA over 10 min at a flow rate of 25 mL/min) to give the title compound (40.29 mg, 90.7 μmol, 12.3% yield, 98.2% purity in HPLC at 220 nm) as white solid. ¹H NMR: (400 MHz, DMSO-d₆) δ 11.0 (s, 1H), 8.27 (s, 1H), 8.20-8.18 (m, 1H), 7.91-7.85 (m, 2H), 7.84-7.82 (m, 1H), 7.68-7.66 (m, 2H), 5.16-5.12 (m, 1H), 4.57-4.40 (m, 2H), 4.05 (s, 3H), 2.95-2.89 (m, 1H), 2.63-2.59 (m, 1H), 2.43-2.39 (m, 1H), 2.04-2.05 (m, 1H). (ESI⁺) m/z: 436.1 (M+H)⁺, (C₂₂H₁ClN₅O₃).

### Example 623

### Synthesis of 3-(5-(5-(4-fluorophenyl)-1-methyl-1H-1,2,4-triazol-3-yl)-1-oxoisoindolin-2-yl)piperidine-2,6-dione

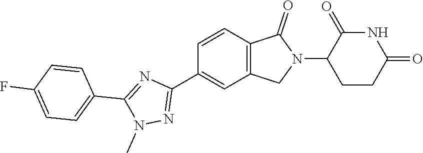 [SMILES: Cn1nc(-c2ccc3c(c2)CN(C2CCC(=O)NC2=O)C3=O)nc1-c1ccc(F)cc1]

A. 3-Bromo-5-(4-fluorophenyl)-1-methyl-1H-1,2,4-triazole: To a solution of 3,5-dibromo-1-methyl-1H-1,2,4-triazole (500 mg, 2.08 mmol, 1.00 eq), (4-fluorophenyl)boronic acid (232 mg, 1.66 mmol, 0.80 eq) and K₂CO₃ (717.18 mg, 5.19 mmol, 2.50 eq) in ACN (6.00 mL) and H₂O (2.00 mL) was added Pd(dppf)Cl₂ (151 mg, 207 μmol, 0.10 eq) under N₂. The mixture was stirred at 40° C. for 12 h under N₂. The mixture was filtered to collect liquid and concentrated under reduced pressure to get residue. The residue was purified by preparative-HPLC (using a Phenomenex luna C18 (150 mm×25 mm 10 μm) and gradient of 28-58% acetonitrile in water containing 0.05% FA over 40 min at a flow rate of 25 mL/min) to give the title compound (200 mg, 781.02 μmol, 37.6% yield, 100% purity in LCMS at 220 nm) as light yellow solid. ¹H NMR: (400 MHz, DMSO-d₆) δ 7.85-7.81 (m, 2H), 7.44-7.39 (m, 2H), 3.93 (s, 3H). (ESI⁺) m/z: 257.0 (M+H)⁺, (C₉H₇BrFN₃).

B. 3-(5-(5-(4-Fluorophenyl)-1-methyl-1H-1,2,4-triazol-3-yl)-1-oxoisoindolin-2-yl)piperidine-2,6-dione: To a solution of 3-bromo-5-(4-fluorophenyl)-1-methyl-1H-1,2,4-triazole (180 mg, 702 μmol, 1.00 eq), 3-(1-oxo-5-(4,4,5,5-tetramethyl-1,3,2-dioxaborolan-2-yl)isoindolin-2-yl)piperidine-2,6-dione (650 mg, 1.76 mmol, 2.50 eq) and K₃PO₄ (298 mg, 1.41 mmol, 2.00 eq) in dioxane (5.00 mL) and H₂O (0.25 mL) was added Ru-Phos-Pd-G₃ (117 mg, 141 μmol, 0.20 eq) under N₂. The mixture was stirred at 100° C. for 2 h under N₂. The mixture was stirred at 100° C. for 2 h under N₂. The mixture was filtered to collect liquid and concentrated under reduced pressure to get residue. The crude product was purified by preparative-HPLC (using a Phenomenex luna C18 (150 mm×25 mm 10 μm) and gradient of 24-54% acetonitrile in water containing 0.5% TFA over 10 min at a flow rate of 25 mL/min) to give the title compound (45.6 mg, 108 μmol, 15.4% yield, 100% purity in HPLC at 220 nm) as white solid. ¹H NMR: (400 MHz, DMSO-d₆) δ 11.0 (s, 1H), 8.27 (s, 1H), 8.20-8.18 (m, 1H), 7.95-7.91 (m, 2H), 7.84-7.82 (m, 1H), 7.46-7.42 (m, 2H), 5.16-5.12 (m, 1H), 4.57-4.40 (m, 2H), 4.03 (s, 3H), 2.93-2.87 (m, 1H), 2.63-2.59 (m, 1H), 2.43-2.39 (m, 1H), 2.05-2.02 (m, 1H). (ESI⁺) m/z: 420.1 (M+H)⁺, (C₂₂H₁₈FN₅O₃).

### Examples 624-689

The compounds of Examples 624-689 were prepared according to Scheme 5 as shown in Examples 555-587.

| | | MW + 1 | Step 1 |
|---|---|---|---|
| Example | Structure | (Observed) | Conditions |
| 624 | 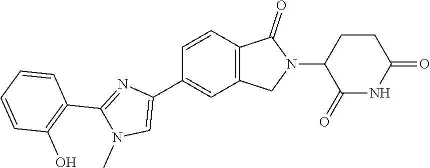 [SMILES: Cn1cc(-c2ccc3c(c2)CN(C2CCC(=O)NC2=O)C3=O)nc1-c1ccccc1O] | 417.3 | 2 |
| 625 | 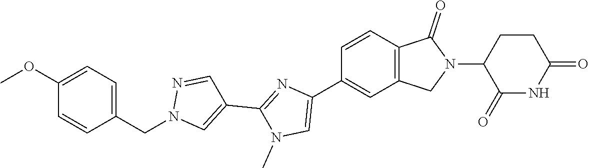 [SMILES: COc1ccc(Cn2cc(-c3nc(-c4ccc5c(c4)CN(C4CCC(=O)NC4=O)C5=O)cn3C)cn2)cc1] | 511.3 | 2 |
| 626 | 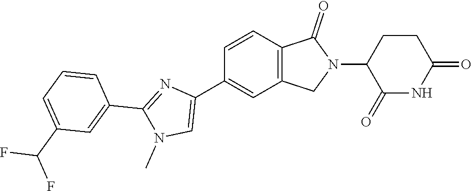 [SMILES: Cn1cc(-c2ccc3c(c2)CN(C2CCC(=O)NC2=O)C3=O)nc1-c1cccc(C(F)F)c1] | 451.2 | 2 |
| 627 | 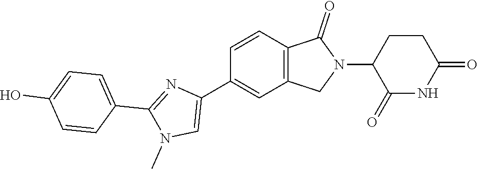 [SMILES: Cn1cc(-c2ccc3c(c2)CN(C2CCC(=O)NC2=O)C3=O)nc1-c1ccc(O)cc1] | 417.3 | 2 |
| 628 | 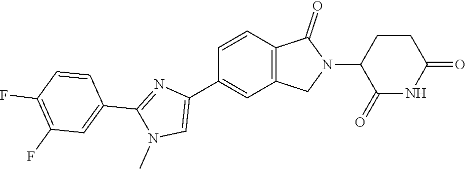 [SMILES: Cn1cc(-c2ccc3c(c2)CN(C2CCC(=O)NC2=O)C3=O)nc1-c1ccc(F)c(F)c1] | 437.2 | 2 |
| 629 | 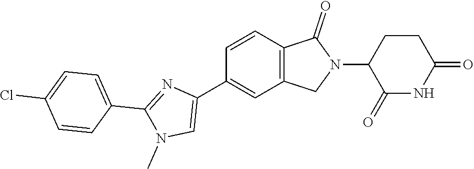 [SMILES: Cn1cc(-c2ccc3c(c2)CN(C2CCC(=O)NC2=O)C3=O)nc1-c1ccc(Cl)cc1] | 435.2 | 2 |
| 630 | 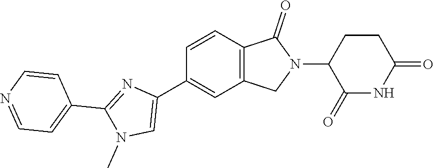 [SMILES: Cn1cc(-c2ccc3c(c2)CN(C2CCC(=O)NC2=O)C3=O)nc1-c1ccncc1] | 402.3 | 2 |
| 631 | 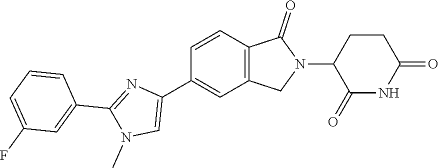 [SMILES: Cn1cc(-c2ccc3c(c2)CN(C2CCC(=O)NC2=O)C3=O)nc1-c1cccc(F)c1] | 419.2 | 2 |
| 632 | 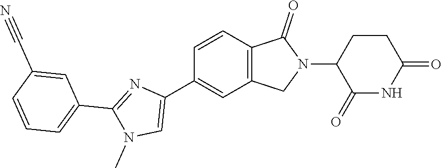 [SMILES: Cn1cc(-c2ccc3c(c2)CN(C2CCC(=O)NC2=O)C3=O)nc1-c1cccc(C#N)c1] | 426.3 | 2 |
| 633 | 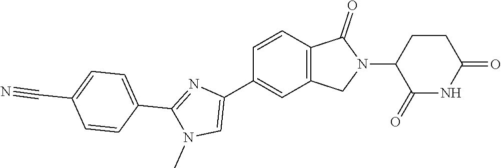 [SMILES: Cn1cc(-c2ccc3c(c2)CN(C2CCC(=O)NC2=O)C3=O)nc1-c1ccc(C#N)cc1] | 426.3 | 2 |
| 634 | 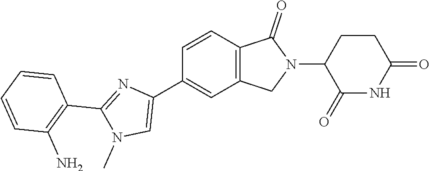 [SMILES: Cn1cc(-c2ccc3c(c2)CN(C2CCC(=O)NC2=O)C3=O)nc1-c1ccccc1N] | 416.3 | 2 |
| 635 | 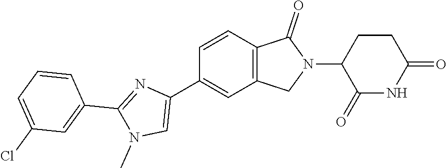 [SMILES: Cn1cc(-c2ccc3c(c2)CN(C2CCC(=O)NC2=O)C3=O)nc1-c1cccc(Cl)c1] | 435.2 | 2 |
| 636 | 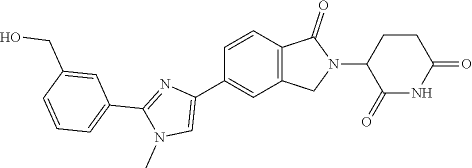 [SMILES: Cn1cc(-c2ccc3c(c2)CN(C2CCC(=O)NC2=O)C3=O)nc1-c1cccc(CO)c1] | 431.3 | 2 |
| 637 | 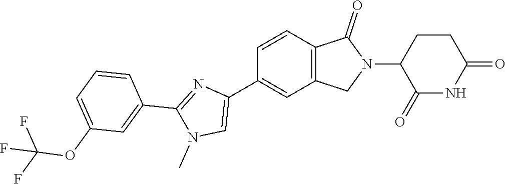 [SMILES: Cn1cc(-c2ccc3c(c2)CN(C2CCC(=O)NC2=O)C3=O)nc1-c1cccc(OC(F)(F)F)c1] | 485.3 | 3 |
| 638 | 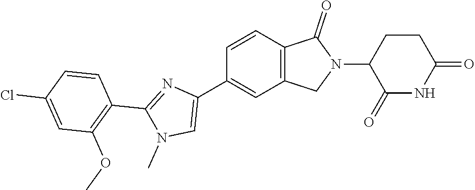 [SMILES: COc1cc(Cl)ccc1-c1nc(-c2ccc3c(c2)CN(C2CCC(=O)NC2=O)C3=O)cn1C] | 465.2 | 2 |
| 639 | 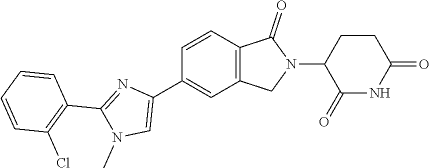 [SMILES: Cn1cc(-c2ccc3c(c2)CN(C2CCC(=O)NC2=O)C3=O)nc1-c1ccccc1Cl] | 435.3 | 2 |
| 640 | 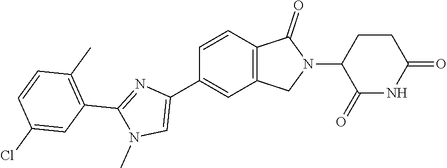 [SMILES: Cc1ccc(Cl)cc1-c1nc(-c2ccc3c(c2)CN(C2CCC(=O)NC2=O)C3=O)cn1C] | 449.2 | 2 |
| 641 | 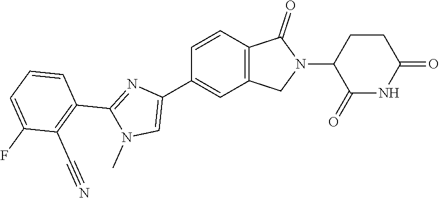 [SMILES: Cn1cc(-c2ccc3c(c2)CN(C2CCC(=O)NC2=O)C3=O)nc1-c1cccc(F)c1C#N] | 444.2 | 2 |
| 642 | 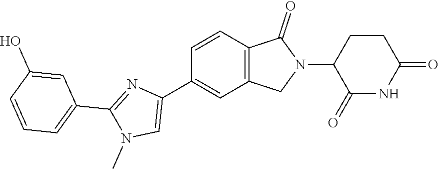 [SMILES: Cn1cc(-c2ccc3c(c2)CN(C2CCC(=O)NC2=O)C3=O)nc1-c1cccc(O)c1] | 417.2 | 1 |
| 643 | 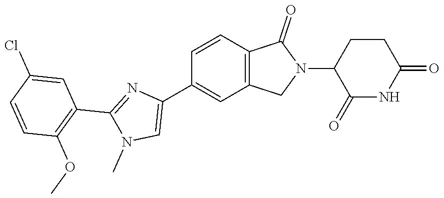 [SMILES: COc1ccc(Cl)cc1-c1nc(-c2ccc3c(c2)CN(C2CCC(=O)NC2=O)C3=O)cn1C] | 465.2 | 2 |
| 644 | 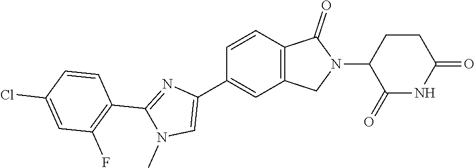 [SMILES: Cn1cc(-c2ccc3c(c2)CN(C2CCC(=O)NC2=O)C3=O)nc1-c1ccc(Cl)cc1F] | 453.2 | 2 |
| 645 | 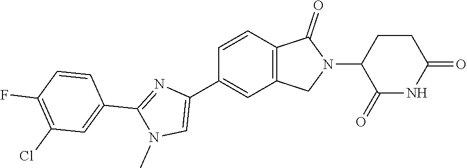 [SMILES: Cn1cc(-c2ccc3c(c2)CN(C2CCC(=O)NC2=O)C3=O)nc1-c1ccc(F)c(Cl)c1] | 453.2 | 2 |
| 646 | 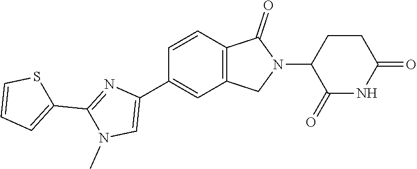 [SMILES: Cn1cc(-c2ccc3c(c2)CN(C2CCC(=O)NC2=O)C3=O)nc1-c1cccs1] | 407.2 | 2 |
| 647 | 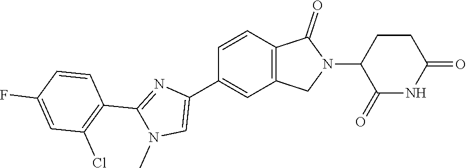 [SMILES: Cn1cc(-c2ccc3c(c2)CN(C2CCC(=O)NC2=O)C3=O)nc1-c1ccc(F)cc1Cl] | 453.2 | 2 |
| 648 | 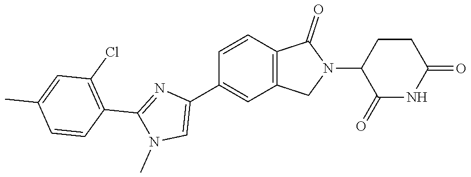 [SMILES: Cc1ccc(-c2nc(-c3ccc4c(c3)CN(C3CCC(=O)NC3=O)C4=O)cn2C)c(Cl)c1] | 449.2 | 2 |
| 649 | 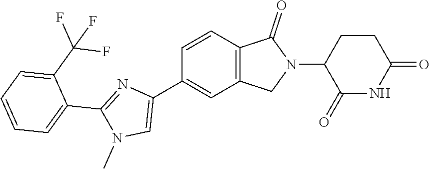 [SMILES: Cn1cc(-c2ccc3c(c2)CN(C2CCC(=O)NC2=O)C3=O)nc1-c1ccccc1C(F)(F)F] | 469.2 | 2 |
| 650 | 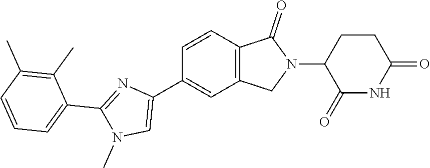 [SMILES: Cc1cccc(-c2nc(-c3ccc4c(c3)CN(C3CCC(=O)NC3=O)C4=O)cn2C)c1C] | 429.3 | 1 |
| 651 | 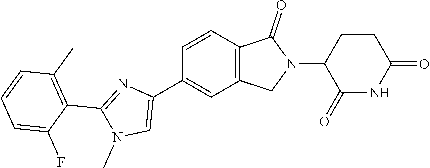 [SMILES: Cc1cccc(F)c1-c1nc(-c2ccc3c(c2)CN(C2CCC(=O)NC2=O)C3=O)cn1C] | 433.3 | 2 |
| 652 | 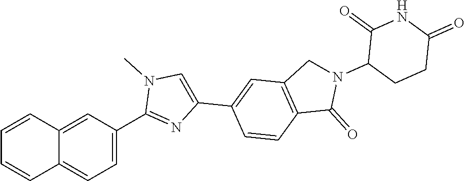 [SMILES: Cn1cc(-c2ccc3c(c2)CN(C2CCC(=O)NC2=O)C3=O)nc1-c1ccc2ccccc2c1] | 451.3 | 2 |
| 653 | 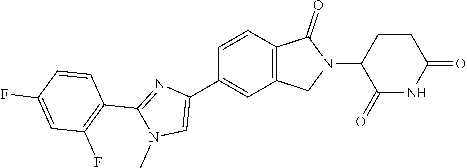 [SMILES: Cn1cc(-c2ccc3c(c2)CN(C2CCC(=O)NC2=O)C3=O)nc1-c1ccc(F)cc1F] | 437.3 | 2 |
| 654 | 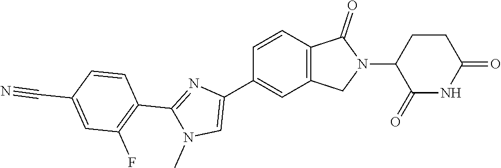 [SMILES: Cn1cc(-c2ccc3c(c2)CN(C2CCC(=O)NC2=O)C3=O)nc1-c1ccc(C#N)cc1F] | 444.2 | 2 |
| 655 | 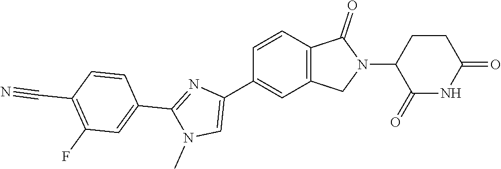 [SMILES: Cn1cc(-c2ccc3c(c2)CN(C2CCC(=O)NC2=O)C3=O)nc1-c1ccc(C#N)c(F)c1] | 444.3 | 2 |
| 656 | 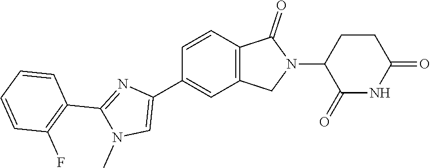 [SMILES: Cn1cc(-c2ccc3c(c2)CN(C2CCC(=O)NC2=O)C3=O)nc1-c1ccccc1F] | 419.2 | 2 |
| 657 | 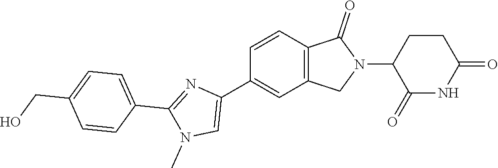 [SMILES: Cn1cc(-c2ccc3c(c2)CN(C2CCC(=O)NC2=O)C3=O)nc1-c1ccc(CO)cc1] | 431.3 | 2 |
| 658 | 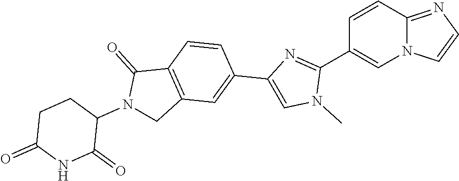 [SMILES: Cn1cc(-c2ccc3c(c2)CN(C2CCC(=O)NC2=O)C3=O)nc1-c1ccc2nccn2c1] | 441.2 | 2 |
| 659 | 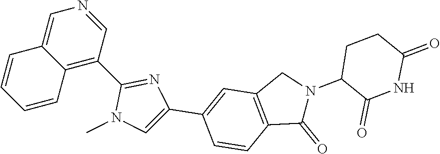 [SMILES: Cn1cc(-c2ccc3c(c2)CN(C2CCC(=O)NC2=O)C3=O)nc1-c1cncc2ccccc12] | 452.3 | 2 |
| 660 | 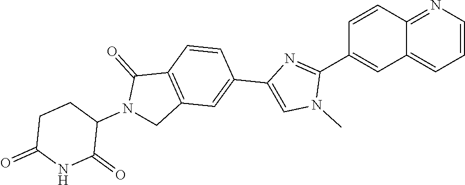 [SMILES: Cn1cc(-c2ccc3c(c2)CN(C2CCC(=O)NC2=O)C3=O)nc1-c1ccc2ncccc2c1] | 452.3 | 2 |
| 661 | 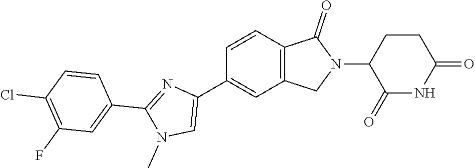 [SMILES: Cn1cc(-c2ccc3c(c2)CN(C2CCC(=O)NC2=O)C3=O)nc1-c1ccc(Cl)c(F)c1] | 453.2 | 2 |
| 662 | 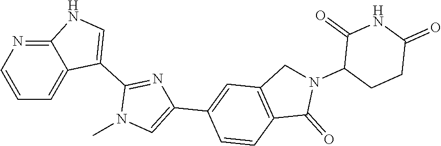 [SMILES: Cn1cc(-c2ccc3c(c2)CN(C2CCC(=O)NC2=O)C3=O)nc1-c1c[nH]c2ncccc12] | 441.3 | 2 |
| 663 | 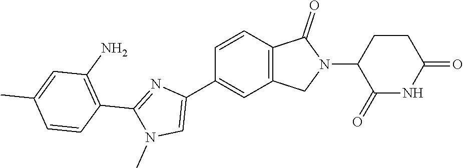 [SMILES: Cc1ccc(-c2nc(-c3ccc4c(c3)CN(C3CCC(=O)NC3=O)C4=O)cn2C)c(N)c1] | 430.3 | 2 |
| 664 | 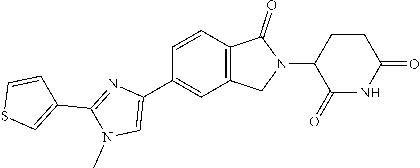 [SMILES: Cn1cc(-c2ccc3c(c2)CN(C2CCC(=O)NC2=O)C3=O)nc1-c1ccsc1] | 407.2 | 2 |
| 665 | 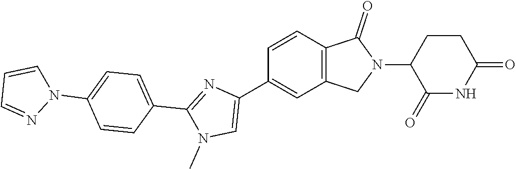 [SMILES: Cn1cc(-c2ccc3c(c2)CN(C2CCC(=O)NC2=O)C3=O)nc1-c1ccc(-n2cccn2)cc1] | 467.3 | 2 |
| 666 | 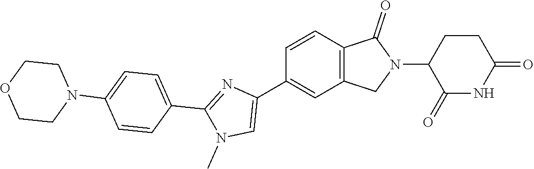 [SMILES: Cn1cc(-c2ccc3c(c2)CN(C2CCC(=O)NC2=O)C3=O)nc1-c1ccc(N2CCOCC2)cc1] | 486.3 | 2 |
| 667 | 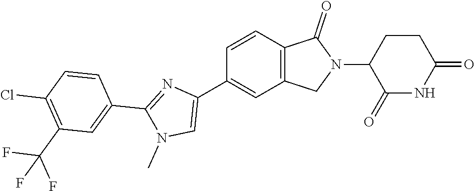 [SMILES: Cn1cc(-c2ccc3c(c2)CN(C2CCC(=O)NC2=O)C3=O)nc1-c1ccc(Cl)c(C(F)(F)F)c1] | 503.3 | 2 |
| 668 | 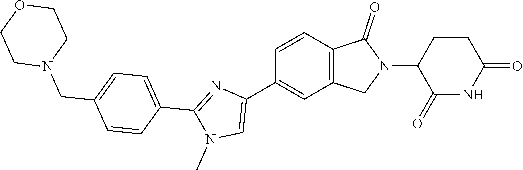 [SMILES: Cn1cc(-c2ccc3c(c2)CN(C2CCC(=O)NC2=O)C3=O)nc1-c1ccc(CN2CCOCC2)cc1] | 500.3 | 2 |
| 669 | 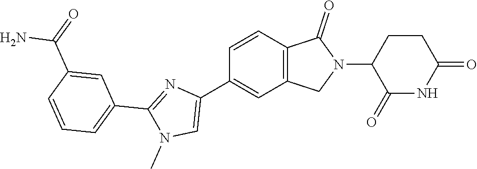 [SMILES: Cn1cc(-c2ccc3c(c2)CN(C2CCC(=O)NC2=O)C3=O)nc1-c1cccc(C(N)=O)c1] | 444.3 | 2 |
| 670 | 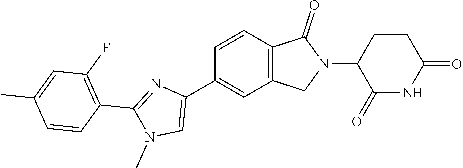 [SMILES: Cc1ccc(-c2nc(-c3ccc4c(c3)CN(C3CCC(=O)NC3=O)C4=O)cn2C)c(F)c1] | 433.3 | 2 |
| 671 | 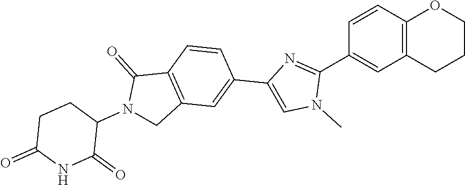 [SMILES: Cn1cc(-c2ccc3c(c2)CN(C2CCC(=O)NC2=O)C3=O)nc1-c1ccc2c(c1)CCCO2] | 457.3 | 2 |
| 672 | 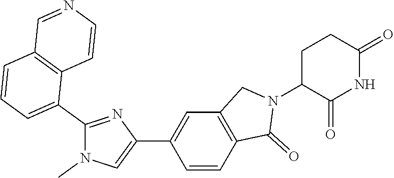 [SMILES: Cn1cc(-c2ccc3c(c2)CN(C2CCC(=O)NC2=O)C3=O)nc1-c1cccc2cnccc12] | 452.3 | 2 |
| 673 | 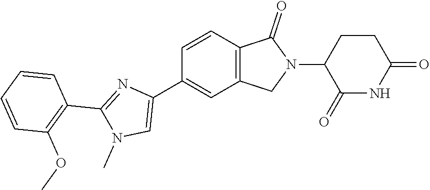 [SMILES: COc1ccccc1-c1nc(-c2ccc3c(c2)CN(C2CCC(=O)NC2=O)C3=O)cn1C] | 431.3 | 2 |
| 674 | 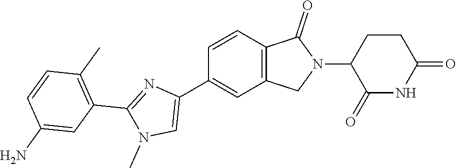 [SMILES: Cc1ccc(N)cc1-c1nc(-c2ccc3c(c2)CN(C2CCC(=O)NC2=O)C3=O)cn1C] | 430.3 | 2 |
| 675 | 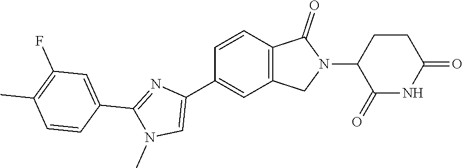 [SMILES: Cc1ccc(-c2nc(-c3ccc4c(c3)CN(C3CCC(=O)NC3=O)C4=O)cn2C)cc1F] | 433.3 | 2 |
| 676 | 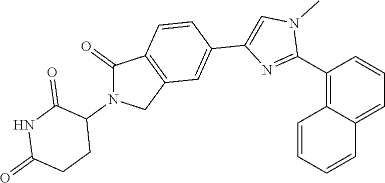 [SMILES: Cn1cc(-c2ccc3c(c2)CN(C2CCC(=O)NC2=O)C3=O)nc1-c1cccc2ccccc12] | 451.3 | 2 |
| 677 | 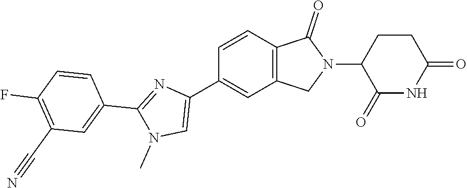 [SMILES: Cn1cc(-c2ccc3c(c2)CN(C2CCC(=O)NC2=O)C3=O)nc1-c1ccc(F)c(C#N)c1] | 444.3 | 2 |
| 678 | 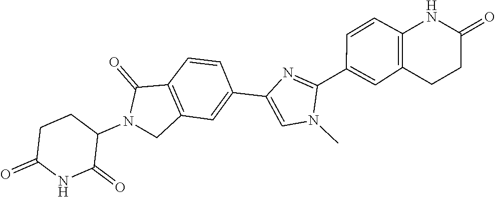 [SMILES: Cn1cc(-c2ccc3c(c2)CN(C2CCC(=O)NC2=O)C3=O)nc1-c1ccc2c(c1)CCC(=O)N2] | 470.3 | 2 |
| 679 | 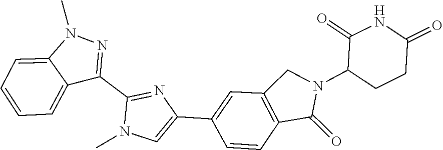 [SMILES: Cn1cc(-c2ccc3c(c2)CN(C2CCC(=O)NC2=O)C3=O)nc1-c1nn(C)c2ccccc12] | 455.3 | 2 |
| 680 | 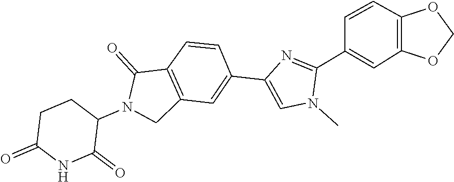 [SMILES: Cn1cc(-c2ccc3c(c2)CN(C2CCC(=O)NC2=O)C3=O)nc1-c1ccc2c(c1)OCO2] | 445.3 | 2 |
| 681 | 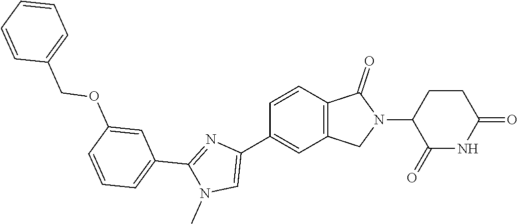 [SMILES: Cn1cc(-c2ccc3c(c2)CN(C2CCC(=O)NC2=O)C3=O)nc1-c1cccc(OCc2ccccc2)c1] | 507.4 | 2 |
| 682 | 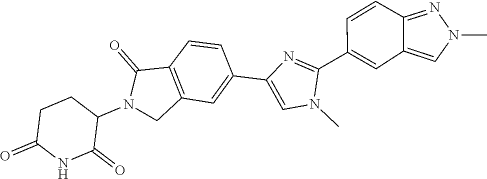 [SMILES: Cn1cc2cc(-c3nc(-c4ccc5c(c4)CN(C4CCC(=O)NC4=O)C5=O)cn3C)ccc2n1] | 455.3 | 2 |
| 683 | 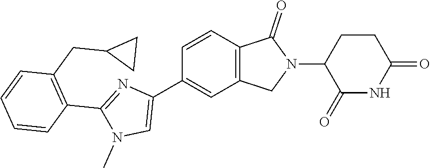 [SMILES: Cn1cc(-c2ccc3c(c2)CN(C2CCC(=O)NC2=O)C3=O)nc1-c1ccccc1CC1CC1] | 457.3 | 2 |
| 684 | 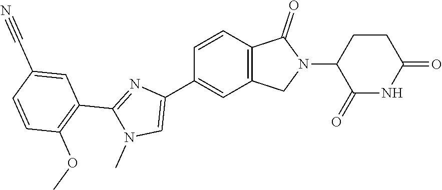 [SMILES: COc1ccc(C#N)cc1-c1nc(-c2ccc3c(c2)CN(C2CCC(=O)NC2=O)C3=O)cn1C] | 456.3 | 2 |
| 685 | 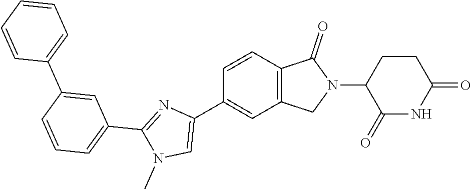 [SMILES: Cn1cc(-c2ccc3c(c2)CN(C2CCC(=O)NC2=O)C3=O)nc1-c1cccc(-c2ccccc2)c1] | 477.3 | 2 |
| 686 | 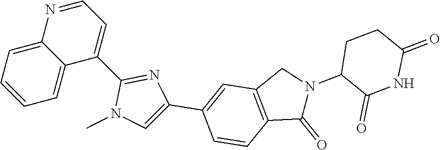 [SMILES: Cn1cc(-c2ccc3c(c2)CN(C2CCC(=O)NC2=O)C3=O)nc1-c1ccnc2ccccc12] | 452.3 | 2 |
| 687 | 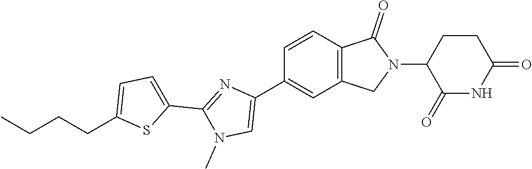 [SMILES: CCCCc1ccc(-c2nc(-c3ccc4c(c3)CN(C3CCC(=O)NC3=O)C4=O)cn2C)s1] | 463.3 | 3 |
| 688 | 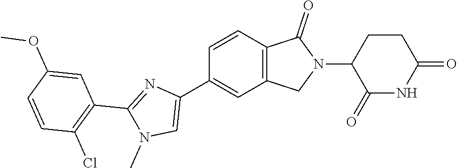 [SMILES: COc1ccc(Cl)c(-c2nc(-c3ccc4c(c3)CN(C3CCC(=O)NC3=O)C4=O)cn2C)c1] | 465.3 | 2 |
| 689 | 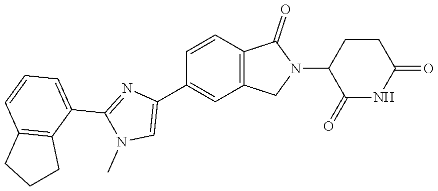 [SMILES: Cn1cc(-c2ccc3c(c2)CN(C2CCC(=O)NC2=O)C3=O)nc1-c1cccc2c1CCC2] | 441.3 | 2 |

### Examples 690-702

The compounds of Examples 690-702 were prepared according to the method of Scheme 8:

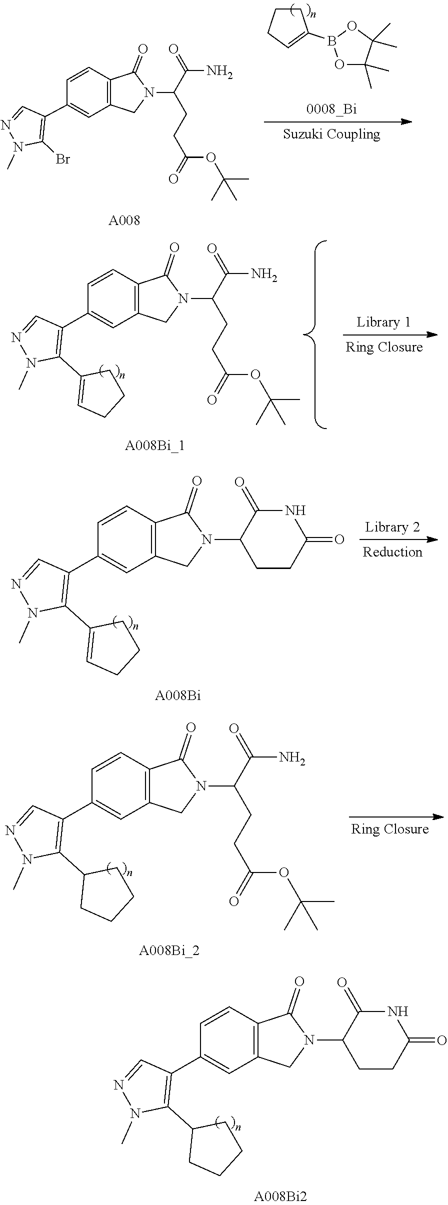

Step 1 Suzuki Coupling

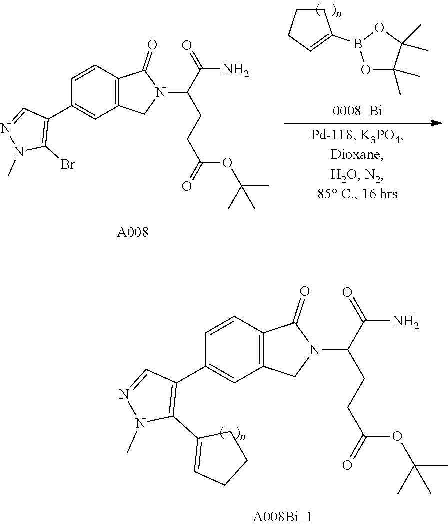

Under N₂, to a solution of A008 (47.6 mg, 0.10 mmol, 1.00 eq.) in Dioxane (1.00 mL) and K₃PO₄ (100 μL, 0.20 mmol, 2 M in H₂O, 2.00 eq.) was added 0008_Bi (0.15 mmol, 1.50 eq.), Pd-118 (3.25 mg, 0.005 mmol, 0.05 eq.). Then the mixture was stirred at 85° C. for 16 hrs. Spot checked by LCMS. The reaction mixture was concentrated under nitrogen flow gas, diluted with H₂O (1.00 mL) and extracted with EA (2.00 mL×3), dried over Na₂SO₄, filtered and concentrated to give A008Bi_1 used for next step.

Step 2: Ring Closure

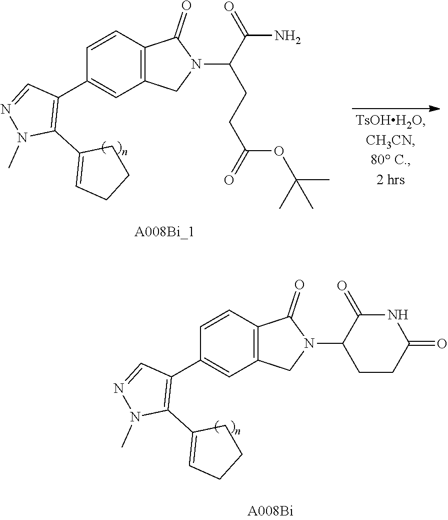

To a solution of A008Bi_1 (0.10 mmol, 1.00 eq.) in CH₃CN (1.00 mL) was added TsOH·H₂O (0.80 mmol, 8.00 eq.). Then the mixture was stirred at 80° C. for 2 hrs. Spot checked by LCMS. The reaction mixture was filtered and purified by prep-HPLC to give desired product.

Step 1 Suzuki Coupling

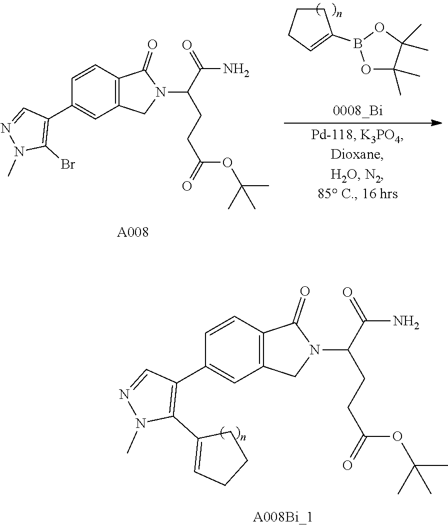

Under N₂, to a solution of A008 (95.2 mg, 0.20 mmol, 1.00 eq.) in Dioxane (2.00 mL) and K₃PO₄ (200 μL, 0.40 mmol, 2 M in H₂O, 2.00 eq.) was added 0008_Bi (0.30 mmol, 1.50 eq.), Pd-118 (6.50 mg, 0.01 mmol, 0.05 eq.). Then the mixture was stirred at 85° C. for 16 hrs. Spot checked by LCMS. The reaction mixture was concentrated under nitrogen flow gas, diluted with H₂O (1.00 mL) and extracted with EA (2.00 mL×3), dried over Na₂SO₄, filtered and concentrated to give A008Bi_1 used for next step.

Step 2 Hydrogenation

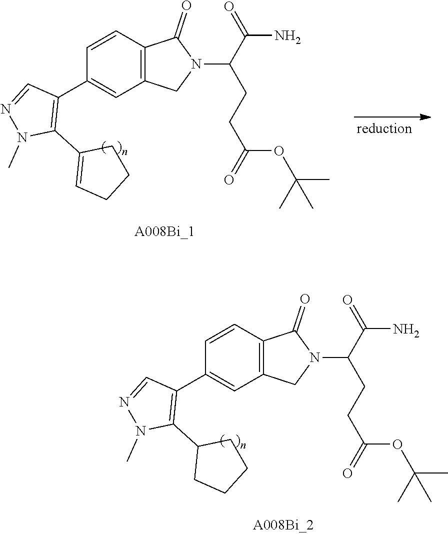

Condition 1

To a solution of A008Bi_1 (0.20 mmol, 1.00 eq.) in THF (1.50 mL) and MeOH (1.50 mL) was added Pd/C (10%, 120 mg, 0.60 eq.) under Ar₂ atmosphere. The suspension was degassed and purged with H₂ for 3 times. The mixture was stirred under H₂ (50 psi) at 50° C. for 16 hrs. Spot checked by LCMS. The mixture was filtered and concentrated to give A008Bi_2 used for next step.

Condition 2

To a solution of A008Bi_1 (0.20 mmol, 1.00 eq.) and AcOH (0.06 mmol, 0.30 eq.) in THF (1.50 mL) and MeOH (1.50 mL) was added Pd/C (10%, 120 mg, 0.60 eq.) under Ar₂ atmosphere. The suspension was degassed and purged with H₂ for 3 times. The mixture was stirred under H₂ (50 psi) at 50° C. for 16 hrs. Spot checked by LCMS. The mixture was filtered and concentrated to give A008Bi_2 used for next step.

Step 3 Ring Closure

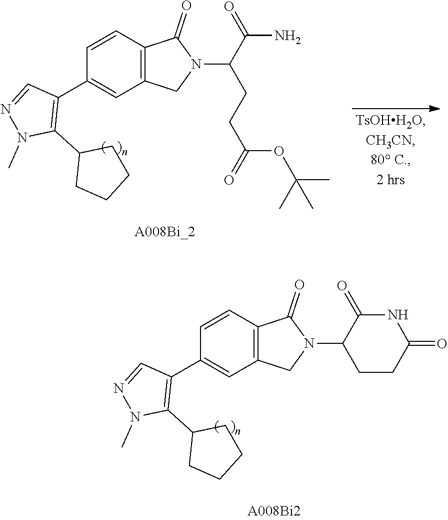

To a solution of A008Bi_2 (0.10 mmol, 1.00 eq.) in CH₃CN (1.00 mL) was added TsOH·H₂O (0.80 mmol, 8.00 eq.). Then the mixture was stirred at 80° C. for 2 hrs. Spot checked by LCMS. The reaction mixture was filtered and purified by prep-HPLC to give desired product.

| | | MW + 1 | Step 2 |
|---|---|---|---|
| Example | Structure | Observed | Conditions |
| 690 | 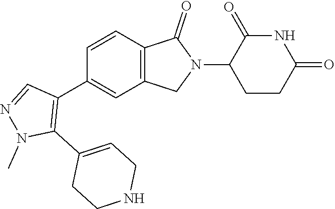 [SMILES: Cn1ncc(-c2ccc3c(c2)CN(C2CCC(=O)NC2=O)C3=O)c1C1=CCNCC1] | 406.3 | |
| 691 | 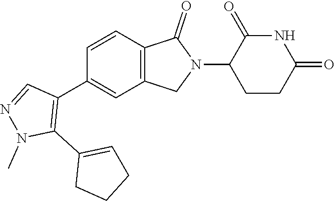 [SMILES: Cn1ncc(-c2ccc3c(c2)CN(C2CCC(=O)NC2=O)C3=O)c1C1=CCCC1] | 391.2 | |
| 692 | 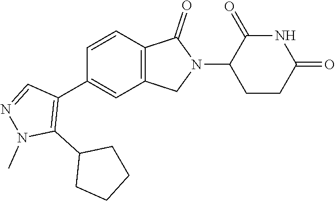 [SMILES: Cn1ncc(-c2ccc3c(c2)CN(C2CCC(=O)NC2=O)C3=O)c1C1CCCC1] | 393.3 | 1 |
| 693 | 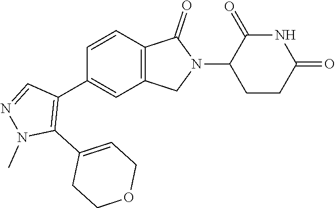 [SMILES: Cn1ncc(-c2ccc3c(c2)CN(C2CCC(=O)NC2=O)C3=O)c1C1=CCOCC1] | 407.2 | |
| 694 | 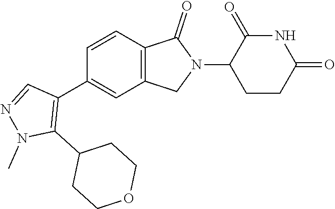 [SMILES: Cn1ncc(-c2ccc3c(c2)CN(C2CCC(=O)NC2=O)C3=O)c1C1CCOCC1] | 409.3 | 2 |
| 695 | 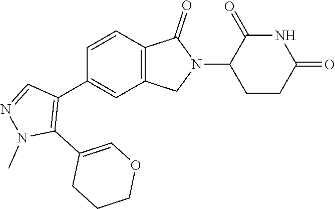 [SMILES: Cn1ncc(-c2ccc3c(c2)CN(C2CCC(=O)NC2=O)C3=O)c1C1=COCCC1] | 407.2 | |
| 696 | 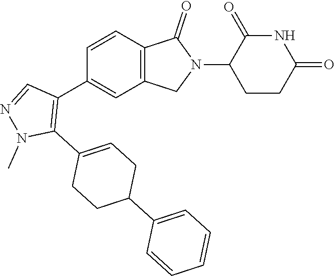 [SMILES: Cn1ncc(-c2ccc3c(c2)CN(C2CCC(=O)NC2=O)C3=O)c1C1=CCC(c2ccccc2)CC1] | 481.3 | |
| 697 | 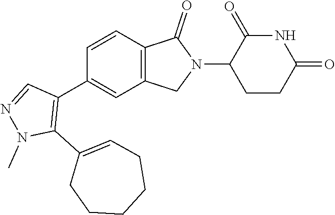 [SMILES: Cn1ncc(-c2ccc3c(c2)CN(C2CCC(=O)NC2=O)C3=O)c1C1=CCCCCC1] | 419.3 | |
| 698 | 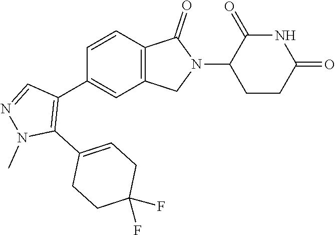 [SMILES: Cn1ncc(-c2ccc3c(c2)CN(C2CCC(=O)NC2=O)C3=O)c1C1=CCC(F)(F)CC1] | 441.3 | |
| 699 | 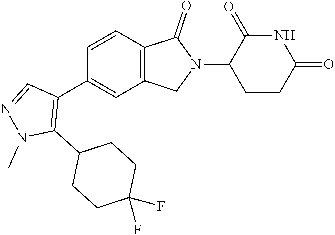 [SMILES: Cn1ncc(-c2ccc3c(c2)CN(C2CCC(=O)NC2=O)C3=O)c1C1CCC(F)(F)CC1] | 443.3 | 2 |
| 700 | 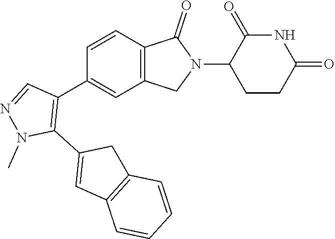 [SMILES: Cn1ncc(-c2ccc3c(c2)CN(C2CCC(=O)NC2=O)C3=O)c1C1=Cc2ccccc2C1] | 439.3 | |
| 701 | 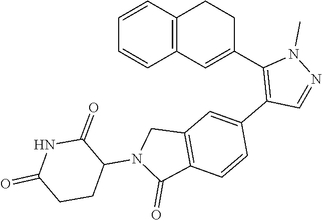 [SMILES: Cn1ncc(-c2ccc3c(c2)CN(C2CCC(=O)NC2=O)C3=O)c1C1=Cc2ccccc2CC1] | 453.3 | |
| 702 | 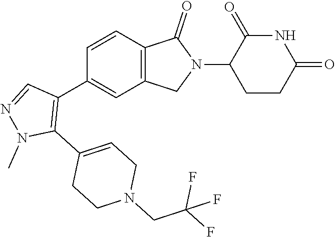 [SMILES: Cn1ncc(-c2ccc3c(c2)CN(C2CCC(=O)NC2=O)C3=O)c1C1=CCN(CC(F)(F)F)CC1] | 488.3 | |

### Example 703

### Synthesis of 3-(5-(1-(difluoromethyl)-5-phenyl-1H-1,2,4-triazol-3-yl)-1-oxoisoindolin-2-yl) piperidine-2,6-dione

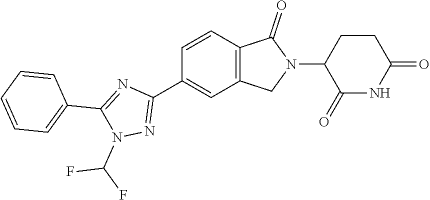 [SMILES: O=C1CCC(N2Cc3cc(-c4nc(-c5ccccc5)n(C(F)F)n4)ccc3C2=O)C(=O)N1]

A. 3-Bromo-5-phenyl-1H-1,2,4-triazole: To a solution of 3,5-dibromo-1H-1,2,4-triazole (500 mg, 2.20 mmol, 1.00 eq), phenylboronic acid (403 mg, 3.31 mmol, 1.50 eq) and CsF (669 mg, 4.41 mmol, 162 μL, 2.00 eq) in MeOH (4.00 mL) was added (PPh₃)₂PdCl₂ (154 mg, 220 μmol, 0.10 eq) under N₂. The mixture was stirred at 110° C. for 1 h in MW under N₂. The reaction mixture was concentrated under reduced pressure to get a residue at 45° C. The residue was purified by column chromatography (SiO₂, Petroleum ether:Ethyl acetate=40:1 to 10:1, TLC:Petroleum ether:Ethyl acetate=10:1, R_{f}=0.30) to give the title compound (440 mg, 1.96 mmol, 29.3% yield, 100% purity in LCMS at 220 nm) as a light yellow oil. (ESI⁺) m/z: 225.9 (M+H)⁺, (C₈H₆BrN₃).

B. 3-Bromo-1-(difluoromethyl)-5-phenyl-1H-1,2,4-triazole: To a solution of 3-bromo-5-phenyl-1H-1,2,4-triazole (440 mg, 1.96 mmol, 1.00 eq) and KF (228 mg, 3.93 mmol, 92.0 μL, 2.00 eq) in ACN (10.0 mL). The mixture was stirred until a colorless oil was formed. Then diethyl (bromodifluoromethyl) phosphonate (576 mg, 2.16 mmol, 1.10 eq) was added to the mixture at 25° C. under N₂. The mixture was stirred at 25° C. for 12 h under N₂. The reaction mixture was concentrated under reduced pressure to get a residue at 45° C. The crude product was purified by column chromatography (SiO₂, Petroleum ether:Ethyl acetate=200:1 to 8:1, TLC:Petroleum ether:Ethyl acetate=8:1,R_{f}=0.55) to give the title compound (100 mg, 347 μmol, 17.7% yield, 95.3% purity in LCMS at 220 nm) as a light yellow oil. ¹HNMR: (400 MHz, DMSO-d₆) δ 7.68-7.63 (m, 2H), 7.51-7.45 (m, 3H), 7.27-6.98 (m, 1H). (ESI⁺) m/z: 274.9 (M+H)⁺, (C₉H₆BrF₂N₃).

C. 3-(5-(1-(Difluoromethyl)-5-phenyl-1H-1,2,4-triazol-3-yl)-1-oxoisoindolin-2-yl)piperidine-2,6-dione: To a solution of 3-bromo-1-(difluoromethyl)-5-phenyl-1H-1,2,4-triazole (80 mg, 291 μmol, 1.00 eq), 3-(1-oxo-5-(4,4,5,5-tetramethyl-1,3,2-dioxaborolan-2-yl)isoindolin-2-yl)piperidine-2,6-dione (216 mg, 583 μmol, 2.00 eq) and K₃PO₄ (185 mg, 875 μmol, 3.00 eq) in dioxane (5.00 mL) and H₂O (0.25 mL) was added Ru-Phos-Pd-G₃ (24.4 mg, 29.1 μmol, 0.10 eq) under N₂. The mixture was stirred at 100° C. for 3 h under N₂. The reaction mixture was concentrated under reduced pressure to get a residue at 45° C. The residue was purified by preparative-HPLC (using a Phenomenex luna C18 (150×25 mm×10 μm) and gradient of 26.0%-56.0% acetonitrile in water containing 0.05% FA over 14 min at a flow rate of 25 mL/min to give the title compound (45.6 mg, 104 μmol, 35.7% yield, 99.8% purity in HPLC at 220 nm) as white solid. ¹HNMR: (400 MHz, DMSO-d₆) δ 11.01 (s, 1H), 8.45-8.28 (m, 2H), 8.26-7.86 (m, 2H), 7.81-7.80 (m, 2H), 7.68-7.65 (m, 3H), 5.17-5.13 (m, 1H), 4.60-4.43 (m, 2H), 2.92-2.88 (m, 1H), 2.64-2.59 (m, 1H), 2.44-2.40 (m, 1H), 2.07-2.04 (m, 1H). (ESI⁺) m/z: 438.1 (M+H)⁺, (C₂₃H₁₇F₂N₅O₃).

### Example 704

### Synthesis of 3-(5-(1-cyclopropyl-2-phenyl-1H-imidazol-4-yl)-1-oxoisoindolin-2-yl)piperidine-2,6-dione

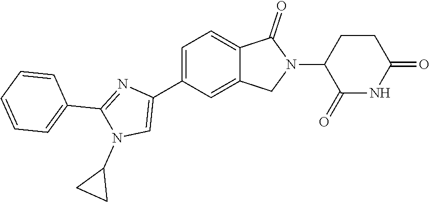 [SMILES: O=C1CCC(N2Cc3cc(-c4cn(C5CC5)c(-c5ccccc5)n4)ccc3C2=O)C(=O)N1]

A. 2,4,5-Tribromo-1-cyclopropyl-1H-imidazole: To a solution of 2,4,5-tribromo-1H-imidazole (5.00 g, 16.4 mmol, 1.00 eq), cyclopropylboronic acid (4.23 g, 49.2 mmol, 3.00 eq), 2-(2-pyridyl)pyridine (2.56 g, 16.4 mmol, 1.00 eq) and Na₂CO₃ (5.22 g, 49.2 mmol, 3.00 eq) in DCE (100 mL) was added Cu(OAc)₂ (2.98 g, 16.4 mmol, 1.00 eq). The mixture was stirred at 70° C. for 12 h. The reaction mixture was concentrated under reduced pressure to get a residue at 45° C. The residue was purified by column chromatography (SiO₂, Petroleum ether:Ethyl acetate=100:1 to 5:1, TLC:Petroleum ether:Ethyl acetate=5:1, R=0.60) to give the title compound (1.00 g, 2.87 mmol, 17.5% yield, 99.0% purity in LCMS at 220 nm) as a white solid. ¹HNMR: (400 MHz, DMSO-d₆) δ 3.23-3.18 (m, 1H), 1.21-1.16 (m, 2H), 1.07-1.03 (m, 2H). (ESI⁺) m/z: 344.6 (M+H)⁺, (C₆H₅Br₃N₂).

B. 4,5-Dibromo-1-cyclopropyl-2-phenyl-1H-imidazole: To a solution of 2,4,5-tribromo-1-cyclopropyl-1H-imidazole (800 mg, 2.32 mmol, 1.00 eq), phenylboronic acid (311 mg, 2.55 mmol, 1.10 eq) and Na₂CO₃ (2 M, 2.32 mL, 2.00 eq) in Tol. (10.0 mL) and MeOH (2.00 mL) was added Pd(PPh₃)₄ (268 mg, 232 μmol, 0.10 eq) under N₂. The mixture was stirred at 65° C. for 12 h under N₂. The reaction mixture was poured into H₂O (50.0 mL) and extracted with Ethyl acetate (3×100 mL). The combined organic layer was washed with brine (3×100 mL), dried over Na₂SO₄, filtered and concentrated. The residue was purified by column chromatography (SiO₂, Petroleum ether:Ethyl acetate=100:1 to 5:1, TLC: Petroleum ether:Ethyl acetate=5:1, R_{f}=0.30) to give the title compound (160 mg, 378 μmol, 16.3% yield, 81.0% purity in LCMS at 220 nm) as a light yellow oil. ¹HNMR: (400 MHz, DMSO-d₆) δ 7.74-7.72 (m, 2H), 7.50-7.47 (m, 3H), 3.61-3.56 (m, 1H), 1.06-1.01 (m, 2H), 0.58-0.56 (m, 2H). (ESI⁺) m/z: 342.8 (M+H)⁺, (C₁₂H₁₀Br₂N₂).

C. 4-Bromo-1-cyclopropyl-2-phenyl-1H-imidazole: To a solution of 4,5-dibromo-1-cyclopropyl-2-phenyl-1H-imidazole (160 mg, 467 μmol, 1.00 eq) in THF (5.00 mL). Then BuLi (2.5 M, 205 μL, 1.10 eq) was added to the reaction mixture at −70° C. under N₂. The mixture was stirred at −70° C. for 1 h under N₂. The reaction mixture was poured into H₂O (20.0 mL) and extracted with Ethyl acetate (3×30.0 mL). The combined organic layer was washed with brine (3×30.0 mL), dried over Na₂SO₄, filtered and concentrated. The residue was purified by prep-TLC (Petroleum ether:Ethyl acetate=10:1,R_{f}=0.40) to give the title compound (100 mg, 353 μmol, 75.5% yield, 93.0% purity in LCMS at 220 nm) as a light yellow oil. ¹HNMR: (400 MHz, DMSO-d₆) δ 7.85-7.83 (m, 2H), 7.48-7.43 (m, 4H), 3.71-3.66 (m, 1H), 0.97-0.95 (m, 2H), 0.85-0.83 (m, 2H). (ESI⁺) m/z: 264.9 (M+H)⁺, (C₁₂H₁₁BrN₂).

D. 3-(5-(1-Cyclopropyl-2-phenyl-1H-imidazol-4-yl)-1-oxoisoindolin-2-yl)piperidine-2,6-dione: To a solution of 4-bromo-1-cyclopropyl-2-phenyl-1H-imidazole (90.0 mg, 342 μmol, 1.00 eq), 3-(1-oxo-5-(4,4,5,5-tetramethyl-1,3,2-dioxaborolan-2-yl)isoindolin-2-yl)piperidine-2,6-dione (379 mg, 1.03 mmol, 3.00 eq) and K₃PO₄ (217 mg, 1.03 mmol, 3.00 eq) in dioxane (5.00 mL) and H₂O (0.25 mL) was added Ru-Phos-Pd-G₃ (28.6 mg, 34.2 μmol, 0.10 eq) under N₂. The mixture was stirred at 100° C. for 2 h under N₂. The reaction mixture was concentrated under reduced pressure to get a residue at 45° C. The residue was purified by preparative-HPLC (using a Phenomenex luna C18 (150×25 mm×10 μm) and gradient of 15.0%-35.0% acetonitrile in water containing 0.05% FA over 14 min at a flow rate of 25 mL/min to give the title compound (7.30 mg, 16.2 μmol, 4.75% yield, 95.8% purity in HPLC at 220 nm) as white solid. ¹HNMR: (400 MHz, DMSO-d₆) δ 11.01 (s, 1H), 8.21-8.01 (m, 2H), 7.99-7.97 (m, 3H), 7.96-7.80 (m, 1H), 7.78-7.58 (s, 3H), 5.16-5.11 (m, 1H), 4.54-4.37 (m, 2H), 3.85-3.81 (m, 1H), 2.96-2.92 (m, 1H), 2.64-2.59 (m, 1H), 2.50-2.42 (m, 1H), 2.05-1.98 (m, 1H), 1.09-0.99 (m, 4H). (ESI⁺) m/z: 427.2 (M+H)⁺, (C₂₅H₂₂N₄O₃).

### Example 705

### Synthesis of 3-(5-(1-cyclopropyl-5-phenyl-1H-1,2,4-triazol-3-yl)-1-oxoisoindolin-2-yl)piperidine-2,6-dione

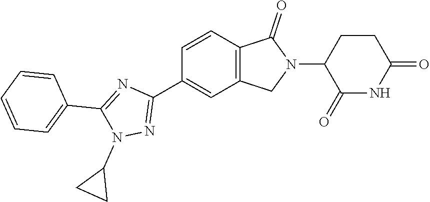 [SMILES: O=C1CCC(N2Cc3cc(-c4nc(-c5ccccc5)n(C5CC5)n4)ccc3C2=O)C(=O)N1]

A. 3,5-Dibromo-1-cyclopropyl-1H-1,2,4-triazole: A solution of 3,5-dibromo-1H-1,2,4-triazole (5.00 g, 22.0 mmol, 1.00 eq), cyclopropylboronic acid (18.9 g, 220 mmol, 10.0 eq), Cu(OAc)₂ (4.80 g, 26.4 mmol, 1.20 eq), 2-(2-pyridyl)pyridine (4.13 g, 26.4 mmol, 1.20 eq) and Na₂CO₃ (5.84 g, 55.1 mmol, 2.50 eq) in dioxane (100 mL) was stirred at 70° C. for 3 h under 02. After that, it was diluted with dichloromethane (100 mL) and washed with saturated aqueous solution of ammonium chloride (100 mL) and brine (100 mL), dried over Na₂SO₄ and concentrated in vacuum to give residue. The residue was purified by column chromatography (SiO₂, Petroleum ether:Ethyl acetate=100:1 to 50:1; TLC, Petroleum ether:Ethyl acetate=3:1, R_{f}=0.40) to give the title compound (6.00 g, 5.99 mmol, 27.2% yield) as colorless oil. ¹H NMR: (400 MHz, CDCl₃) δ 3.52-3.47 (m, 1H), 1.28-1.25 (m, 2H), 1.18-1.16 (m, 2H). (ESI⁺) m/z: 267.8 (M+H)⁺, (C₅H₅Br₂N₃).

B. 3-Bromo-1-cyclopropyl-5-phenyl-1H-1,2,4-triazole: To a solution of 3,5-dibromo-1-cyclopropyl-1,2,4-triazole (0.40 g, 1.50 mmol, 1.00 eq), phenylboronic acid (146 mg, 1.20 mmol, 0.80 eq) in MeOH (4.00 mL) was added CsF (455 mg, 3.00 mmol, 110 μL, 2.00 eq) and (PPh₃)₂PdCl₂ (105 mg, 149 μmol, 0.10 eq) under N₂. The reaction mixture was stirred at 110° C. under N₂ in MW for 0.5 h. The reaction mixture was concentrated in vacuum to give residue. The residue was purified by column chromatography (SiO₂, Petroleum ether:Ethyl acetate=100:1 to 20:1; TLC, Petroleum ether:Ethyl acetate=5:1, R_{f}=0.40) to give the title compound (0.75 g, 2.84 mmol, 47.3% yield) as colorless oil. ¹H NMR: (400 MHz, CDCl₃) δ 7.89-7.87 (m, 2H), 7.53-7.51 (m, 3H), 3.70-3.64 (m, 1H), 1.25-1.21 (m, 2H), 1.15-1.12 (m, 2H). (ESI⁺) m/z: 264.4 (M+H)⁺, (C₁₁H₁₀BrN₃).

C. 3-(5-(1-Cyclopropyl-5-phenyl-1H-1,2,4-triazol-3-yl)-1-oxoisoindolin-2-yl)piperidine-2,6-dione: To a solution of 3-bromo-1-cyclopropyl-5-phenyl-1,2,4-triazole (0.40 g, 1.51 mmol, 1.00 eq), 3-[1-oxo-5-(4,4,5,5-tetramethyl-1,3,2-dioxaborolan-2-yl)isoindolin-2-yl]piperidine-2,6-dione (1.40 g, 3.79 mmol, 2.50 eq) in dioxane (8.00 mL) and H₂O (0.40 mL) was added Ru-Phos-Pd-G₃ (126 mg, 151 μmol, 0.10 eq), K₃PO₄ (642 mg, 3.03 mmol, 2 eq) under N₂. The reaction mixture was stirred at 100° C. for 2 h under N₂. The reaction mixture was concentrated in vacuum to give residue. The residue was purified by column chromatography (SiO₂, Dichloromethane:Methanol=100:1 to 50:1; TLC, Dichloromethane:Methanol=10:1, R_{f}=0.40) and preparative-HPLC (using a Welch Xtimate C18 (150 mm×25 mm×10 μm) and gradient of 20-50% acetonitrile in water containing 0.05% TFA over 11 min at a flow rate of 25.0 mL/min) to give the title compound (8.92 mg, 20.7 μmol, 1.37% yield, 99.5% purity in HPLC at 220 nm) as white solid. ¹H NMR: (400 MHz, DMSO-d₆) δ 11.0 (s, 1H), 8.26 (s, 1H), 8.19 (d, J=6.8 Hz, 1H), 8.03-8.00 (m, 2H), 7.82 (d, J=8.0 Hz, 1H), 7.60-7.54 (m, 3H), 5.16-5.11 (m, 1H), 4.57-4.40 (m, 2H), 4.00-3.96 (m, 1H), 2.95-2.89 (m, 1H), 2.63-2.58 (m, 1H), 2.43-2.39 (m, 1H), 2.05-2.03 (m, 1H), 1.13-1.10 (m, 4H). (ESI⁺) m/z: 428.1 (M+H)⁺, (C₂₄H₂IN₅O₃).

### Example 706

### Synthesis of 3-(5-(5-((1R,5S)-8-azabicyclo[3.2.1]octan-8-yl)-1-methyl-1H-1,2,4-triazol-3-yl)-1-oxoisoindolin-2-yl)piperidine-2,6-dione

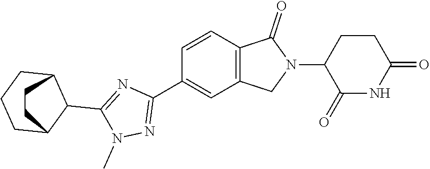 [SMILES: Cn1nc(-c2ccc3c(c2)CN(C2CCC(=O)NC2=O)C3=O)nc1C1[C@@H]2CCC[C@H]1CC2]

A. (1R,5S)-8-(3-Bromo-1-methyl-1H-1,2,4-triazol-5-yl)-8-azabicyclo[3.2.1]octane: To a solution of 3, 5-dibromo-1-methyl-1H-1,2,4-triazole (500 mg, 2.08 mmol, 1.00 eq) and K₂CO₃ (860 mg, 6.23 mmol, 3.00 eq) in NMP (8.00 mL) was added (1R,5S)-8-azabicyclo[3.2.1]octane (306 mg, 2.08 mmol, 1.00 eq, HCl). The mixture was stirred at 160° C. in MW for 0.75 h. The mixture was poured into H₂O (20.0 mL) and extracted with Ethyl acetate (3×15.0 mL). The combined organic layer was washed with saturated NaCl aqueous (5×15.0 mL), dried over Na₂SO₄, and concentrated under reduced pressure to get residue. The crude product was purified by preparative-HPLC (using a Phenomenex luna C18 (150 mm×25 mm 10 μm) and gradient of 31-61% acetonitrile in water containing 0.05% TFA over 10 min at a flow rate of 25 mL/min) to give the title compound (340 mg, 1.25 mmol, 30.2% yield, 100% purity in LCMS at 220 nm) as white solid. ¹H NMR: (400 MHz, CDCl₃) δ 3.40-3.36 (m, 2H), 2.84 (s, 3H), 2.38-2.06 (m, 2H), 2.02-1.98 (m, 4H), 1.74-0.88 (m, 4H). (ESI⁺) m/z: 271.0 (M+H)⁺, (C₁₀H₁₅BrN₄).

B. 3-(5-(1-methyl-2-((1r,4r)-4-methylcyclohexyl)-1H-imidazol-4-yl)-1-oxoisoindolin-2-yl)piperidine-2,6-dione: To a solution of (1S,5R)-8-(5-bromo-2-methyl-1,2,4-triazol-3-yl)-8-azabicyclo[3.2.1]octane (320 mg, 1.18 mmol, 1.00 eq), 3-[1-oxo-5-(4,4,5,5-tetramethyl-1,3,2-dioxaborolan-2-yl)isoindolin-2-yl]piperidine-2,6-dione (873 mg, 2.36 mmol, 2.00 eq) and K₃PO₄ (501 mg, 2.36 mmol, 2.00 eq) in dioxane (5.00 mL) and H₂O (0.25 mL) was added Ru-Phos-Pd-G₃ (197 mg, 236 μmol, 0.20 eq) under N₂. The mixture was stirred at 100° C. for 2 h. The mixture was filtered to collected liquid and concentrated under reduced pressure to get residue. The crude product was purified by preparative-HPLC (using a Phenomenex luna C18 (150 mm×25 mm 10 μm) and gradient of 16-46% acetonitrile in water containing 0.05% TFA over 14 min at a flow rate of 25 mL/min) to give the title compound (440 mg, 990 μmol, 83.9% yield, 97.7% purity in HPLC at 220 nm) as white solid. 1H NMR: (400 MHz, DMSO-d₆) δ 10.9 (s, 1H), 8.10 (s, 1H), 8.06-8.03 (m, 1H), 7.77-7.75 (m, 1H), 5.14-5.10 (m, 1H), 4.53-4.34 (m, 2H), 4.18-4.14 (m, 2H), 3.78 (s, 3H), 2.91-2.87 (m, 1H), 2.40-2.37 (m, 1H), 2.03-2.00 (m, 1H), 1.87-1.76 (m, 3H), 1.74-1.61 (m, 5H), 1.50-1.47 (m, 3H). (ESI⁺) m/z: 435.2 (M+H)⁺, (C₂₃H₂₆N₆O₃).

### Example 707

### Synthesis of 3-(5-(1-methyl-5-(4-(trifluoromethyl)piperidin-1-yl)-1H-1,2,4-triazol-3-yl)-1-oxoisoindolin-2-yl)piperidine-2,6-dione

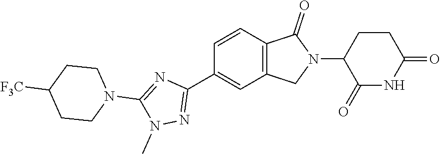 [SMILES: Cn1nc(-c2ccc3c(c2)CN(C2CCC(=O)NC2=O)C3=O)nc1N1CCC(C(F)(F)F)CC1]

A. 1-(3-Bromo-1-methyl-1H-1,2,4-triazol-5-yl)-4-(trifluoromethyl)piperidine: A solution of 3,5-dibromo-1-methyl-1H-1,2,4-triazole (0.50 g, 2.08 mmol, 1.00 eq), 4-(trifluoromethyl)piperidine (317 mg, 2.08 mmol, 1.00 eq) and K₂CO₃ (573 mg, 4.15 mmol, 2.00 eq) in NMP (8.00 mL) was stirred at 145° C. for 0.75 h in MW. The reaction mixture was poured into H₂O (20.0 mL), extracted with ethyl acetate (3×30.0 mL), dried over Na₂SO₄ and concentrated in vacuum to give residue. The residue was purified by column chromatography (SiO₂, Dichloromethane:Methanol=100:1 to 50:1; TLC, Dichloromethane:Methanol=20:1, R_{f}=0.40) to give the title compound (0.40 g, 1.28 mmol, 61.5% yield) as white solid. ¹H NMR: (400 MHz, CDCl₃) δ 3.68 (s, 3H), 3.50-3.47 (m, 2H), 2.97-2.90 (m, 2H), 2.24-2.21 (m, 1H), 2.00-1.95 (m, 2H), 1.81-1.74 (m, 2H). m/z: 314.9 (M+H)⁺, (C₉H₁₂BrF₃N₄).

B. 3-(5-(1-Methyl-5-(4-(trifluoromethyl)piperidin-1-yl)-1H-1,2,4-triazol-3-yl)-1-oxoisoindolin-2-yl)piperidine-2,6-dione: To a solution of 1-(3-bromo-1-methyl-1H-1,2,4-triazol-5-yl)-4-(trifluoromethyl)piperidine (0.40 g, 1.28 mmol, 1.00 eq) and 3-[1-oxo-5-(4,4,5,5-tetramethyl-1,3,2-dioxaborolan-2-yl)isoindolin-2-yl]piperidine-2,6-dione (1.18 g, 3.19 mmol, 2.50 eq) in dioxane (5.00 mL) and H₂O (0.25 mL) was added Ru-Phos-Pd-G₃ (106 mg, 127 μmol, 0.10 eq), K₃PO₄ (542 mg, 2.55 mmol, 2.00 eq) under N₂. The reaction mixture was stirred at 100° C. for 2 h under N₂. The reaction mixture was concentrated in vacuum to give residue. The residue was purified by column chromatography (SiO₂, Dichloromethane:Methanol=100:1 to 80:1; TLC, Dichloromethane:Methanol=10:1, R_{f}=0.50) and preparative-HPLC (using a Welch Xtimate C18 (200 mm×40 mm×10 μm) and gradient of 20-50% acetonitrile in water containing 0.05% TFA over 15 min at a flow rate of 25.0 mL/min) to give the title compound (257 mg, 530 μmol, 41.5% yield, 98.3% purity in HPLC at 220 nm) as white solid. ¹H NMR: (400 MHz, DMSO-d₆) δ 11.0 (s, 1H), 8.13 (s, 1H), 8.07 (d, J=8.0 Hz, 1H), 7.77 (d, J=8.0 Hz, 1H), 5.14-5.10 (m, 1H), 4.53-4.36 (m, 2H), 3.74 (s, 3H), 3.60-3.57 (m, 2H), 3.00-2.91 (m, 3H), 2.62-2.58 (m, 2H), 2.45-2.41 (m, 1H), 2.05-2.03 (m, 1H), 1.92-1.89 (m, 2H), 1.70-1.66 (m, 2H). (ESI⁺) m/z: 477.2 (M+H)⁺, (C₂₂H₂₃F₃N₆O₃).

### Example 708

### Synthesis of 3-(5-(1-methyl-5-((S)-2-methylpiperidin-1-yl)-1H-1,2,4-triazol-3-yl)-1-oxoisoindolin-2-yl)piperidine-2,6-dione

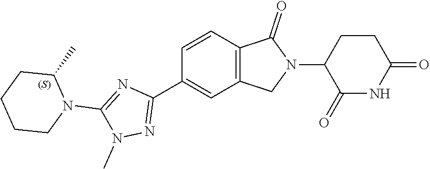

A. (S)-1-(3-Bromo-1-methyl-1H-1,2,4-triazol-5-yl)-2-methylpiperidine: To a solution of 3,5-dibromo-1-methyl-1H-1,2,4-triazole (500 mg, 2.08 mmol, 1.00 eq), (S)-2-methylpiperidine (226 mg, 2.28 mmol, 269 μL, 1.10 eq) and K₂CO₃ (573 mg, 4.15 mmol, 2.00 eq) in NMP (10.0 mL). The mixture was stirred at 160° C. for 2 h in MW under N₂. The reaction mixture was poured into H₂O (50.0 mL) and extracted with Ethyl acetate (3×50.0 mL). The combined organic layer was washed with brine (3×50.0 mL), dried over Na₂SO₄, filtered and concentrated. The residue was purified by preparative-HPLC (using a Phenomenex luna C18 (150×25 mm×10 μm) and gradient of 34.0%-54.0% acetonitrile in water containing 0.50% TFA over 17 min at a flow rate of 25 mL/min) to give the title compound (280 mg, 1.07 mmol, 25.8% yield, 99.4% purity in LCMS at 220 nm) as light yellow oil. ¹H NMR: (400 MHz, DMSO-d₆) δ3.62-3.57 (m, 3H), 3.29-3.26 (m, 1H), 3.08-3.05 (m, 1H), 2.87-2.84 (m, 1H), 1.75-1.67 (m, 2H), 1.59-1.55 (m 2H), 1.41-1.39 (m, 2H), 0.96-0.95 (m, 3H). (ESI⁺) m/z: 261.0 (M+H)⁺, (C₉H₁₅BrN₄).

B. 3-(5-(1-Methyl-5-((S)-2-methylpiperidin-1-yl)-1H-1,2,4-triazol-3-yl)-1-oxoisoindolin-2-yl)piperidine-2,6-dione: To a solution of (S)-1-(3-bromo-1-methyl-1H-1,2,4-triazol-5-yl)-2-methylpiperidine (140 mg, 0.54 mmol, 1.00 eq), 3-(1-oxo-5-(4,4,5,5-tetramethyl-1,3,2-dioxaborolan-2-yl)isoindolin-2-yl)piperidine-2,6-dione (0.50 g, 1.35 mmol, 2.50 eq) and K₃PO₄ (344 mg, 1.62 mmol, 3.00 eq) in dioxane (5.00 mL) and H₂O (0.25 mL) was added Ru-Phos-Pd-G₃ (45.1 mg, 54.0 μmol, 0.10 eq) under N₂. The mixture was stirred at 100° C. for 2 h under N₂. The reaction mixture was concentrated under reduced pressure to get a residue at 45° C. The residue was purified by preparative-HPLC (using a Phenomenex luna C18 (150×25 mm×5 μm) and gradient of 25.0%-55.0% acetonitrile in water containing 0.50% TFA over 14 min at a flow rate of 25 mL/min) to give the title compound (131 mg, 310 μmol, 57.5% yield, 99.8% purity in HPLC at 220 nm) as white solid. ¹H NMR: (400 MHz, DMSO-d₆) δ 11.00-10.99 (m, 1H), 8.14-8.13 (m, 1H), 8.09-8.07 (m, 1H), 7.78-7.76 (m, 1H), 5.14-5.10 (m, 1H), 4.53-4.37 (m, 2H), 3.73 (s, 3H), 3.35-3.34 (m, 1H), 3.12-2.94 (m, 1H), 2.93-2.88 (m, 2H), 2.63-2.54 (m, 1H), 2.46-2.42 (m, 1H), 2.09-2.03 (m, 1H), 1.77-1.65 (m, 2H), 1.64-1.62 (m, 2H), 1.45-1.44 (m, 2H), 1.01-1.00 (m, 3H). (ESI⁺) m/z: 423.2 (M+H)⁺, (C₂₂H₂₆N₆O₃).

### Example 709

### Synthesis of 3-(5-(1-methyl-5-((R)-2-methylpiperidin-1-yl)-1H-1,2,4-triazol-3-yl)-1-oxoisoindolin-2-yl)piperidine-2,6-dione

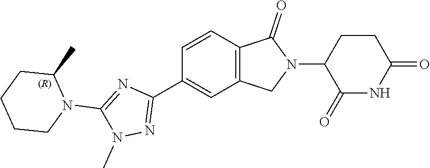

A. (R)-1-(3-Bromo-1-methyl-1H-1,2,4-triazol-5-yl)-2-methylpiperidine: To a solution of 3,5-dibromo-1-methyl-1H-1,2,4-triazole (500 mg, 2.08 mmol, 1.00 eq), (R)-2-methylpiperidine (205 mg, 2.08 mmol, 1.00 eq) in NMP (10.0 mL) was added K₂CO₃ (574 mg, 4.15 mmol, 2.00 eq). The mixture was stirred at 160° C. in MW for 0.75 h. The mixture was poured into H₂O (20.0 mL) and extracted with Ethyl acetate (3×15.0 mL). The combined organic layer was washed with saturated NaCl aqueous (5×15.0 mL), dried over Na₂SO₄, and concentrated under reduced pressure to get residue. The crude product was purified by preparative-HPLC (using a Phenomenex luna C18 (150 mm×25 mm 10 μm) and gradient of 32-62% acetonitrile in water containing 0.05% TFA over 14 min at a flow rate of 25 mL/min) to give the title compound (230 mg, 845 μmol, 20.3% yield, 95.3% purity in LCMS at 220 nm) as brown solid. ¹H NMR: (400 MHz, DMSO-d₆) δ 3.69 (s, 3H), 3.28-3.26 (m, 1H), 3.04-3.01 (m, 1H), 2.97-2.94 (m, 1H), 1.82-1.64 (m, 4H), 1.42-1.40 (m, 2H), 0.99 (s, 3H). (ESI⁺) m/z: 261.0 (M+H)⁺, (C₉H₁₅BrN₄).

B. 3-(5-(1-Methyl-5-((R)-2-methylpiperidin-1-yl)-1H-1,2,4-triazol-3-yl)-1-oxoisoindolin-2-yl)piperidine-2,6-dione: To a solution of (R)-1-(3-bromo-1-methyl-1H-1,2,4-triazol-5-yl)-2-methylpiperidine (200 mg, 771 μmol, 1.00 eq), 3-(1-oxo-5-(4,4,5,5-tetramethyl-1,3,2-dioxaborolan-2-yl)isoindolin-2-yl)piperidine-2,6-dione (714 mg, 1.93 mmol, 2.50 eq) and K₃PO₄ (327 mg, 1.54 mmol, 2.00 eq) in dioxane (5.00 mL) and H₂O (0.25 mL) was added Ru-Phos-Pd-G₃ (129 mg, 154 μmol, 0.20 eq) under N₂. The mixture was stirred at 100° C. for 2 h under N₂. The mixture was filtered to collect liquid and concentrated under reduced pressure to get residue. The crude product was purified by preparative-HPLC (using a Phenomenex luna C18 (150 mm×25 mm 10 μm) and gradient of 18-48% acetonitrile in water containing 0.5% FA over 12 min at a flow rate of 25 mL/min) to give the title compound (236 mg, 548 μmol, 71.0% yield, 97.8% purity in HPLC at 220 nm) as white solid. 1H NMR: (400 MHz, DMSO-d₆) δ 10.9 (s, 1H), 8.14 (s, 1H), 8.09-8.07 (m, 1H), 7.78-7.76 (m, 1H), 5.15-5.10 (m, 1H), 4.53-4.41 (m, 2H), 3.78 (s, 3H), 3.35-3.34 (m, 1H), 3.12-2.94 (m, 1H), 2.91-2.88 (m, 2H), 2.63-2.58 (m, 1H), 1.77-1.75 (m, 1H), 1.73-1.70 (m, 1H), 1.65-1.62 (m, 4H), 1.45-1.42 (m, 2H), 1.01 (s, 3H). (ESI⁺) m/z: 423.1 (M+H)⁺, (C₂₂H₂₆N₆O₃).

### Example 710

### Synthesis of 3-(1-oxo-5-(5-phenyl-1-(2,2,2-trifluoroethyl)-1H-pyrazol-3-yl)isoindolin-2-yl)piperidine-2,6-dione

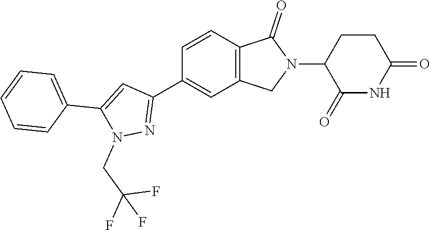 [SMILES: O=C1CCC(N2Cc3cc(-c4cc(-c5ccccc5)n(CC(F)(F)F)n4)ccc3C2=O)C(=O)N1]

A. 3-Iodo-5-phenyl-1-(2,2,2-trifluoroethyl)-1H-pyrazole: To a solution of 3-iodo-5-phenyl-1H-pyrazole (6.00 g, 16.9 mmol, 1.00 eq) in DMF (120 mL) was added 2,2,2-trifluoroethyl trifluoromethanesulfonate (4.33 g, 18.6 mmol, 1.10 eq) and Cs₂CO₃ (16.5 g, 50.9 mmol, 3.00 eq). The mixture was stirred at 25° C. for 12 h. The reaction mixture was poured into saturated NaCl aqueous solution (200 mL) and extracted with ethyl acetate (3×200 mL). The combined organic layer was washed with brine (3×200 mL), dried over Na₂SO₄, filtered and the filtrate was concentrated in vacuum to give a residue. The residue was purified by column chromatography (SiO₂, Petroleum ether:Ethyl acetate=100:1 to 10: 1, R_{f}=0.40 (Petroleum ether:Ethyl acetate=10:1)) and by preparative-TLC (Petroleum ether:Ethyl acetate=10:1, R_{f}=0.40) to give the title compound (620 mg, 1.53 mmol, 8.99% yield, 86.7% purity in HPLC at 220 nm) as yellow oil. ¹H NMR: (400 MHz, DMSO-d₆) δ 7.55-7.46 (m, 5H), 6.72 (s, 1H), 5.07-5.00 (m, 2H). (ESI⁺) m/z: 353.1 (M+H)⁺, (C₁₁H₈F₃IN₂).

B. 3-(1-Oxo-5-(5-phenyl-1-(2,2,2-trifluoroethyl)-1H-pyrazol-3-yl)isoindolin-2-yl)piperidine-2,6-dione: To a solution of 3-iodo-5-phenyl-1-(2,2,2-trifluoroethyl)pyrazole (600 mg, 1.48 mmol, 1.00 eq) in dioxane (6.00 mL) and H₂O (0.30 mL) was added 3-[1-oxo-5-(4,4,5,5-tetramethyl-1,3,2-dioxaborolan-2-yl)isoindolin-2-yl]piperidine-2,6-dione (1.37 g, 3.69 mmol, 2.50 eq), Ru-Phos-Pd-G₃ (123 mg, 147 μmol, 0.10 eq) and K₃PO₄ (627 mg, 2.95 mmol, 2.00 eq). The mixture was stirred at 100° C. for 2 h under N₂. After the reaction was completed, the reaction mixture was filtered and the filtrate was concentrated in vacuum to give a residue. The residue was purified by preparative-TLC (Dichloromethane:Methanol=10:1, R_{f}=0.50) and by preparative-HPLC (using a Phenomenex Luna C18 (150 mm×25 mm×10 μm) and gradient of 33-63% acetonitrile in water containing 0.5% FA over 10 min at a flow rate of 25.0 mL/min) to give the title compound (85.4 mg, 182 μmol, 12.3% yield, 100% purity in HPLC at 220 nm) as a white solid. ¹H NMR: (400 MHz, DMSO-d₆) δ 10.9 (s, 1H), 8.11 (s, 1H), 8.03 (d, J=8.0 Hz, 1H), 7.80 (d, J=8.0 Hz, 1H), 7.58-7.52 (m, 5H), 7.13 (s, 1H), 5.16-5.07 (m, 3H), 4.47 (dd, J=51.6, 17.2 Hz, 2H), 2.98-2.89 (m, 1H), 2.61-2.59 (m, 1H), 2.45-2.39 (m, 1H), 2.06-2.03 (m, 1H). (ESI⁺) m/z: 469.4 (M+H)⁺, (C₂₄H₁₉F₃N₄O₃).

### Example 711

### Synthesis of 3-[5-[2-(1-adamantyl)-1-methyl-imidazol-4-yl]-1-oxo-isoindolin-2-yl]piperidine-2,6-dione

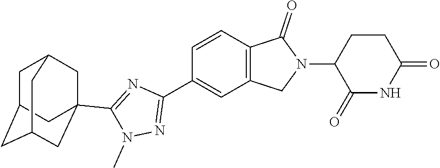 [SMILES: Cn1nc(-c2ccc3c(c2)CN(C2CCC(=O)NC2=O)C3=O)nc1C12CC3CC(CC(C3)C1)C2]

A. N-(2,2-Dimethoxyethyl)-N-methyl-adamantane-1-carboxamide: To a solution of adamantane-1-carboxylic acid (10.0 g, 55.4 mmol, 1.00 eq) in DMF (50.0 mL) was added 2,2-dimethoxy-N-methyl-ethanamine (7.93 g, 66.5 mmol, 8.56 mL, 1.20 eq), HOBt (9.00 g, 66.5 mmol, 1.20 eq), the mixture was cooled to 0° C., the EDCI (12.7 g, 66.5 mmol, 1.20 eq) was added, then the mixture was stirred at 25° C. for 10 h. The reaction mixture was poured into H₂O (100 mL) and extracted with ethyl acetate (100 mL×3). The combined layer was dried over anhydrous Na₂SO₄, filtered and concentrated under reduced pressure to give a residue. The residue was purified by column chromatography (SiO₂, Petroleum ether:Ethyl acetate=100/1 to 50/1, Petroleum ether:Ethyl acetate=2/1, R_{f}=0.70) to give the title compound (11.0 g, 39.0 mmol, 70.4% yield) as a white solid. ¹H NMR: (400 MHz, DMSO-d₆) δ 4.43-4.40 (m, 1H), 3.33 (s, 2H), 3.27 (s, 6H), 3.13 (s, 3H), 1.98-1.90 (m, 10H), 1.66 (s, 5H). (ESI⁺) m/z: 281.2 (M+H)⁺, (C₁₆H₂₇NO₃).

B. 2-(1-Adamantyl)-1-methyl-imidazole: To a solution of N-(2,2-dimethoxyethyl)-N-methyl-adamantane-1-carboxamide (11.0 g, 39.0 mmol, 1.00 eq) in AcOH (220 mL) was added NH₄OAc (60.2 g, 781 mmol, 20.0 eq), the mixture was stirred at 125° C. for 10 h. The reaction solution was adjusted to pH=7 with 10 M NaOH aqueous solution and extracted with DCM (30.0 ml×3), wash with brine. The combined layer was dried over anhydrous Na₂SO₄, filtered and concentrated under reduced pressure to give a residue. The residue was purified by column chromatography (SiO₂, Petroleum ether:Ethyl acetate=100/1 to 2/1, Petroleum ether:Ethyl acetate=1/1, R_{f}=0.50) to give the title compound (2.30 g, 8.92 mmol, 22.8% yield, 83.9% purity in LCMS at 220 nm) as a brown solid. ¹H NMR: (400 MHz, CDCl₃-d₆) δ 6.90 (s, 1H), 6.73 (s, 1H), 3.80 (s, 3H), 2.13-2.03 (m, 9H), 1.79-1.69 (m, 6H). (ESI⁺) m/z: 217.0 (M+H)⁺, (C₁₄H₂₀N₂).

C. 2-(1-Adamantyl)-4,5-dibromo-1-methyl-imidazole (three reactions were carried out in parallel): To a solution of 2-(1-adamantyl)-1-methyl-imidazole (300 mg, 1.37 mmol, 1.00 eq) in THF (5.00 mL) was added NBS (537 mg, 3.02 mmol, 2.20 eq) at 0° C. then the mixture was stirred at 25° C. for 2 h. The reaction mixture was concentrated under vacuum to give the title compound (1.00 g, 2.54 mmol, 61.7% yield, 95.1% purity in LCMS at 220 nm) as a yellow solid. (ESI⁺) m/z: 374.8 (M+H)⁺, (C₁₄H₁₁Br₂N₂).

D. 2-(1-Adamantyl)-4-bromo-1-methyl-imidazole: A solution of 2-(1-adamantyl)-4,5-dibromo-1-methyl-imidazole (700 mg, 1.87 mmol, 1.00 eq) in THF (5.00 mL), the mixture was cooled to −78° C., the mixture was added n-BuLi (2.50 M, 1.50 mL, 2.00 eq), then the mixture was stirred at −78° C. for 2 h. The mixture was poured into H₂O (30.0 mL) and extracted with ethyl acetate (3×40.0 mL). The combined organic layer was washed with saturated NaCl aqueous (3×40.0 mL), dried over Na₂SO₄ and concentrated under reduced pressure to get residue. The crude product was purified by Pre-TLC (Petroleum ether:Ethyl acetate=5:1, R_{f}=0.4) to give the title compound (500 mg, 1.61 mmol, 85.8% yield, 94.8% purity in LCMS at 220 nm) as a yellow solid. ¹H NMR: (400 MHz, CDCl₃-d₆) δ 6.69 (s, 1H), 3.76 (s, 3H), 2.10 (s, 9H), 1.77 (s, 6H). (ESI⁺) m/z: 296.8 (M+H)⁺, (C₁₄H₁₉N₂Br).

E. 3-[5-[2-(1-adamantyl)-1-methyl-imidazol-4-yl]-1-oxo-isoindolin-2-yl]piperidine-2,6-dione (three reactions were carried out in parallel): To a solution of 2-(1-adamantyl)-4-bromo-1-methyl-imidazole (20 mg, 64.22 μmol, 1 eq), 3-[1-oxo-5-(4,4,5,5-tetramethyl-1,3,2-dioxaborolan-2-yl)isoindolin-2-yl]piperidine-2,6-dione (59.4 mg, 160 μmol, 2.50 eq), K₃PO₄ (27.2 mg, 128 μmol, 2.00 eq) in dioxane (1.50 mL) and H₂O (0.07 mL) was added RuPhos Pd G₃ (5.37 mg, 6.42 μmol, 0.10 eq), the mixture was stirred at 100° C. for 2 h. The reaction mixture was concentrated under vacuum. The crude product was purified by preparative-HPLC (using a Phenomenex luna C18 (150 mm×25 mm, 10 m) and gradient of 4%-34% acetonitrile in water containing 0.05% FA over 10 min at a flow rate of 25 mL/min) to give the title compound (10.7 mg, 22.3 μmol, 11.6% yield, 95.1% purity in HPLC at 220 nm) as a white solid. ¹H NMR: (400 MHz, DMSO-d₆) δ 10.9 (s, 1H), 7.89 (s, 1H), 7.82 (d, J=8 Hz, 1H), 7.65 (d, J=8 Hz, 1H), 7.62 (s, 1H), 5.12-5.07 (m, 1H), 4.48-4.30 (m, 2H), 3.82 (s, 3H), 2.96-2.86 (m, 1H), 2.58 (s, 1H), 2.41-2.37 (m, 1H), 2.05 (d, J=16 Hz, 10H), 1.76 (s, 6H). (ESI⁺) m/z: 459.1 (M+H)⁺, (C₂₇H₃₀N₄O₃).

### Example 712

### Synthesis of 3-(5-(5-methyl-1-phenyl-1H-1, 2, 4-triazol-3-yl)-1-oxoisoindolin-2-yl) piperidine-2, 6-dione

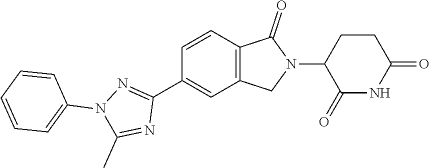 [SMILES: Cc1nc(-c2ccc3c(c2)CN(C2CCC(=O)NC2=O)C3=O)nn1-c1ccccc1]

A. 3-Bromo-5-methyl-1-phenyl-1H-1, 2, 4-triazole: To a solution of 3-bromo-5-methyl-TH-1,2,4-triazole (500 mg, 3.08 mmol, 1.00 eq), phenylboronic acid (1.50 g, 12.3 mmol, 4.00 eq), Na₂CO₃ (818 mg, 7.72 mmol, 2.50 eq) and Cu(OAc)₂ (673 mg, 3.70 mmol, 1.20 eq) in dioxane (16.0 mL) was added TMEDA (430 mg, 3.70 mmol, 559 μL, 1.20 eq). Then the mixture was stirred at 70° C. for 10 h. The reaction was poured into water (30.0 mL) and extracted with EtOAc (3×30.0 mL). The combined organic layers were dried over by Na₂SO₄, filtered and concentrated under reduced pressure to give a residue. The residue was purified by preparative-TLC (Petroleum ether:Ethyl acetate=5:1, R_{f}=0.25) to give the title compound (350 mg, 1.47 mmol, 47.6% yield) was obtained as yellow oil. ¹H NMR: (400 MHz, CDCl₃) δ 7.53-7.37 (m, 5H), 2.53 (s, 3H). (ESI⁺) m/z: 237.9 (M+H)⁺, (C₉H₈BrN₃).

B. 3-(5-(5-methyl-1-phenyl-1H-1, 2, 4-triazol-3-yl)-1-oxoisoindolin-2-yl) piperidine-2, 6-dione: To a solution of 3-bromo-5-methyl-1-phenyl-1H-1, 2, 4-triazole (150 mg, 630 μmol, 1.00 eq), 3-(1-oxo-5-(4,4,5,5-tetramethyl-1,3,2-dioxaborolan-2-yl)isoindolin-2-yl)piperidine-2,6-dione (583 mg, 1.58 mmol, 2.50 eq) and K₃PO₄ (401 mg, 1.89 mmol, 3.00 eq) in dioxane (5.00 mL) and H₂O (0.25 mL) was added Ru-Phos-Pd-G₃ Ru-Phos-Pd-G₃ (52.7 mg, 63.0 μmol, 0.10 eq) under N₂. The mixture was stirred at 100° C. for 2 h under N₂. The reaction mixture was concentrated under reduced pressure to get residue. The residue was purified by preparative-HPLC (using a Phenomenex luna C18 (150×25 mm×10 μm) and gradient of 19%-49% acetonitrile in water containing 0.50% TFA over 15 min at a flow rate of 25 mL/min) to give the title compound (89.7 mg, 223 μmol, 35.4% yield, 100% purity in HPLC at 220 nm) was obtained as white solid. ¹H NMR: (400 MHz, DMSO-d₆) δ 11.0 (s, 1H), 8.27 (s, 1H), 8.23-8.21 (m, 1H), 7.84-7.82 (m, 1H), 7.69-7.67 (m, 2H), 7.63-7.55 (m, 3H), 5.17-5.12 (m, 1H), 4.57-4.40 (m, 2H), 3.91 (s, 3H), 2.97-2.89 (m, 1H), 2.63-2.55 (m, 1H), 2.49-2.42 (m, 1H), 2.05-2.03 (m, 1H). (ESI⁺) m/z: 419.1 (M+H)⁺, (C₂₃H₁₉FN₄O₃).

### Example 713

### Synthesis of 3-(5-(1-methyl-5-(pyrrolidin-1-yl)-1H-1,2,4-triazol-3-yl)-1-oxoisoindolin-2-yl)piperidine-2,6-dione

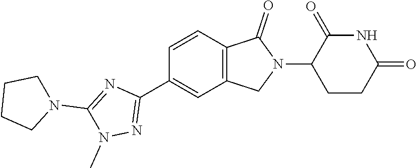 [SMILES: Cn1nc(-c2ccc3c(c2)CN(C2CCC(=O)NC2=O)C3=O)nc1N1CCCC1]

A. 3-Bromo-1-methyl-5-(pyrrolidin-1-yl)-1H-1,2,4-triazole: To a solution of 3,5-dibromo-1-methyl-1,2,4-triazole (500 mg, 2.08 mmol, 1.00 eq) in NMP (10.0 mL) was added pyrrolidine (147 mg, 2.08 mmol, 173 μL, 1.00 eq) and K₂CO₃ (573 mg, 4.15 mmol, 2.00 eq). The mixture was stirred in a microwave reactor at 140° C. for 0.75 h. The reaction mixture was poured into saturated NaCl aqueous solution (20.0 mL) and extracted with ethyl acetate (3×20.0 mL). The combined organic layer was washed with brine (3×20.0 mL), dried over Na₂SO₄, filtered and the filtrate was concentrated in vacuum to get a residue. The residue was purified by column chromatography (SiO₂, Petroleum ether:Ethyl acetate=100:1 to 1:1,R_{f}=0.50 (Petroleum ether:Ethyl acetate=1:1)) to give the title compound (300 mg, 1.28 mmol, 59.2% yield, 98.8% purity in HPLC at 220 nm) as yellow oil. ¹H NMR: (400 MHz, DMSO-d₆) δ 3.69 (s, 3H), 3.47-3.42 (m, 4H), 1.89-1.84 (m, 4H). (ESI⁺) m/z: 232.1 (M+H)⁺, (C₇H₁₁BrN₄).

B. 3-(5-(1-Methyl-5-(pyrrolidin-1-yl)-1H-1,2,4-triazol-3-yl)-1-oxoisoindolin-2-yl)piperidine-2,6-dione: To a solution of 3-bromo-1-methyl-5-pyrrolidin-1-yl-1,2,4-triazole (500 mg, 1.93 mmol, 1.00 eq) in dioxane (5.00 mL) and H₂O (0.25 mL) was added 3-[1-oxo-5-(4,4,5,5-tetramethyl-1,3,2-dioxaborolan-2-yl)isoindolin-2-yl]piperidine-2,6-dione (1.78 g, 4.81 mmol, 2.50 eq), Ru-Phos-Pd-G₃ (161 mg, 192 μmol, 0.10 eq) and K₃PO₄ (817 mg, 3.85 mmol, 2.00 eq) under N₂. The mixture was stirred at 100° C. for 2 h under N₂. After the reaction was completed, the reaction mixture was filtered and the filtrate was concentrated in vacuum to give a residue. The residue was purified by preparative-TLC (Dichloromethane:Methanol=10:1, R_{f}=0.50) and by preparative-HPLC (using a Phenomenex Luna C18 (200 mm×40 mm×10 μm) and gradient of 1-30% acetonitrile in water containing 0.5% TFA over 15 min at a flow rate of 25.0 mL/min) to give the title compound (52.8 mg, 129 μmol, 6.71% yield, 96.4% purity in HPLC at 220 nm) as a white solid. ¹H NMR: (400 MHz, DMSO-d₆) δ 10.9 (s, 1H), 8.10 (s, 1H), 8.05 (d, J=1.2 Hz, 1H), 7.75 (d, J=8.0 Hz, 1H), 5.12 (dd, J=13.2, 5.2 Hz, 1H), 4.44 (dd, J=51.2, 18.0 Hz, 2H), 3.81 (s, 3H), 3.57-3.53 (m, 4H), 2.96-2.87 (m, 1H), 2.61 (m, 1H), 2.40 (m, 1H), 2.05-1.98 (m, 1H), 1.94-1.90 (m, 4H). (ESI⁺) m/z: 395.4 (M+H)⁺, (C₂₀H₂₂N₆O₃).

### Example 714

### Synthesis of 3-(5-(1-methyl-2-(1-methylcyclohexyl)-1H-imidazol-4-yl)-1-oxoisoindolin-2-yl)piperidine-2,6-dione

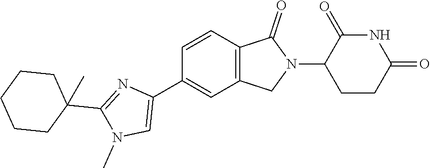 [SMILES: Cn1cc(-c2ccc3c(c2)CN(C2CCC(=O)NC2=O)C3=O)nc1C1(C)CCCCC1]

A. N-(2,2-Dimethoxyethyl)-N,1-dimethylcyclohexane-1-carboxamide: To a solution of 1-methylcyclohexane-1-carboxylic acid (5.00 g, 35.1 mmol, 1.00 eq), 2,2-dimethoxy-N-methylethan-1-amine (5.03 g, 42.2 mmol, 5.42 mL, 1.20 eq) and HOBt (5.70 g, 42.2 mmol, 1.20 eq) in DMF (50.0 mL) was added EDCI (8.09 g, 42.2 mmol, 1.20 eq). The mixture was stirred at 25° C. for 12 h. The reaction mixture was poured into H₂O (300 mL) and extracted with Ethyl acetate (3×300 mL). The combined organic layer was washed with brine (3×300 mL), dried over Na₂SO₄, filtered and concentrated. The residue was purified by column chromatography (SiO₂, Petroleum ether:Ethyl acetate=100:1 to 5:1, TLC: Petroleum ether:Ethyl acetate=5:1, R_{f}=0.40) to give the title compound (16.1 g, 66.1 mmol, 62.2% yield) as yellow oil. ¹H NMR: (400 MHz, CDCl₃) δ 4.56-4.52 (m, 1H), 4.44-4.42 (m, 2H), 4.41 (s, 6H), 3.17 (s, 3H), 1.52-1.36 (m, 10H), 1.33-1.32 (m, 3H), (ESI⁺) m/z: 244 (M+H)⁺, (C₁₃H₂₅NO₃).

B. 1-Methyl-2-(1-methylcyclohexyl)-1H-imidazole: To a solution of N-(2,2-dimethoxyethyl)-N,1-dimethylcyclohexane-1-carboxamide (10.0 g, 41.0 mmol, 1.00 eq) in AcOH (200 mL) was added NH₄OAc (63.3 g, 821 mmol, 20.0 eq) under N₂. The mixture was stirred at 110° C. for 12 h under N₂. The mixture was quenched with 10N NaOH solution until pH=7 and extracted into Ethyl acetate (3×300 mL). The combined organic layer was washed with brine (3×300 mL), dried over Na₂SO₄, filtered and concentrated. The residue was purified by column chromatography (SiO₂, Dichloromethane:Methanol=200:1 to 15:1, TLC:Dichloromethane:Methanol=15:1, R_{f}=0.30) to give the title compound (300 mg, 1.46 mmol, 3.55% yield, 86.7% purity in LCMS at 220 nm) as light yellow oil. ¹H NMR: (400 MHz, DMSO-d₆) δ 6.94 (s, 1H), 6.69 (s, 1H), 368 (s, 3H), 1.47-1.34 (m, 10H), 1.19 (s, 3H) (ESI⁺) m/z: 179.1 (M+H)⁺, (C₁₁H₁₁N₂).

C. 4,5-Dibromo-1-methyl-2-(1-methylcyclohexyl)-1H-imidazole: To a solution of 1-methyl-2-(1-methylcyclohexyl)-1H-imidazole (100 mg, 560 μmol, 1.00 eq) in DMF (5.00 mL) was added NBS (199 mg, 1.12 mmol, 2.00 eq) at 0° C. The mixture was stirred at 25° C. for 2 h. The reaction mixture was poured into saturated Na₂S₂O₃ and K₂CO₃ aqueous (100 mL) and extracted with Ethyl acetate (3×50.0 mL). The combined organic layer was washed with brine (3×50.0 mL), dried over Na₂SO₄, filtered and concentrated. The residue was purified by TLC (Petroleum ether:Ethyl acetate=5:1, R_{f}=0.40) to give the title compound (60.0 mg, 178 μmol, 31.8% yield, 100% purity in LCMS at 220 nm) as light yellow oil. (ESI⁺) m/z: 338 (M+H)⁺, (C₁₁H₁₆Br₂N₂).

D. 4-Bromo-1-methyl-2-(1-methylcyclohexyl)-1H-imidazole: To a solution of 4,5-dibromo-1-methyl-2-(1-methylcyclohexyl)-1H-imidazole (60 mg, 178 μmol, 1.00 eq) in THF (2.00 mL). Then n-BuLi (2.50 M, 78.5 μL, 1.10 eq) was added to the reaction mixture at −78° C. under N₂. The mixture was stirred at −78° C. for 1 h under N₂. The reaction mixture was poured into H₂O (10.0 mL) and extracted with Ethyl acetate (3×30.0 mL). The combined organic layer was washed with brine (3×30.0 mL), dried over Na₂SO₄, filtered and concentrated. The residue was purified by TLC (Petroleum ether:Ethyl acetate=5:1, R_{f}=0.30) to give the title compound (30.0 mg, 90.5 μmol, 50.7% yield, 77.6% purity in LCMS at 220 nm)) as light yellow oil. ¹H NMR: (400 MHz, CDCl₃) δ 6.69 (s, 1H), 3.69 (s, 3H), 4.41 (s, 6H), 1.59-1.43 (m, 10H), 1.28 (s, 3H), (ESI⁺) m/z: 259.0 (M+H)⁺, (C₁₁H₁₇BrN₂).

E. 3-(5-(1-Methyl-2-(1-methylcyclohexyl)-1H-imidazol-4-yl)-1-oxoisoindolin-2-yl)piperidine-2,6-dione: To a solution of 4-bromo-1-methyl-2-(1-methylcyclohexyl)-1H-imidazole (30.0 mg, 116 μmol, 1.00 eq), 3-(1-oxo-5-(4,4,5,5-tetramethyl-1,3,2-dioxaborolan-2-yl)isoindolin-2-yl)piperidine-2,6-dione (107 mg, 291 μmol, 2.50 eq) and K₃PO₄ (74.2 mg, 349 μmol, 3.00 eq) in dioxane (1.00 mL) and H₂O (0.05 mL) was added Ru-Phos-Pd-G₃ (9.76 mg, 11.6 μmol, 0.10 eq) under N₂. The mixture was stirred at 100° C. for 2 h under N₂. The reaction mixture was concentrated under reduced pressure to get a residue at 45° C. The residue was purified by preparative-HPLC (using a Phenomenex luna C18 (150×25 mm×5 μm) and gradient of 13.0%-33.0% acetonitrile in water containing 0.05% FA over 3 min at a flow rate of 25 mL/min to give the title compound (14.7 mg, 33.1 μmol, 29.9% yield, 99.7% purity in HPLC at 220 nm) as white solid. ¹H NMR: (400 MHz, DMSO-d₆) δ 11.0-11.0 (m, 1H), 8.01-7.80 (m, 4H), 5.17-5.10 (m, 1H), 4.53-4.44 (m, 2H), 4.02-3.89 (m, 3H), 2.93-2.92 (m, 1H), 2.63-2.59 (m, 1H), 2.44-2.41 (m, 1H), 2.28-2.23 (m, 2H), 2.10-2.02 (m, 1H), 1.67-1.45 (m, 8H), 1.42-1.37 (m, 3H). (ESI⁺) m/z: 421.2 (M+H)⁺, (C₂₄H₂₈N₄O₃).

### Example 715

### Synthesis of 3-(5-(1-isopropyl-2-phenyl-1H-imidazol-4-yl)-1-oxoisoindolin-2-yl)piperidine-2,6-dione

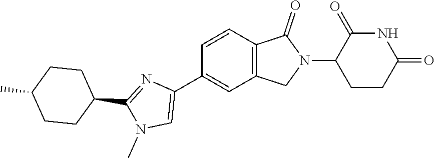 [SMILES: Cn1cc(-c2ccc3c(c2)CN(C2CCC(=O)NC2=O)C3=O)nc1[C@H]1CC[C@H](C)CC1]

A. (1r,4r)-N-(2,2-Dimethoxyethyl)-N,4-dimethylcyclohexane-1-carboxamide: To a solution of (1r,4r)-4-methylcyclohexane-1-carboxylic acid (5.00 g, 35.1 mmol, 1.00 eq), 2,2-dimethoxy-N-methylethan-1-amine (5.03 g, 42.2 mmol, 5.42 mL, 1.20 eq) and HOBt (5.70 g, 42.2 mmol, 1.20 eq) in DMF (50.0 mL) was added EDCI (8.09 g, 42.2 mmol, 1.20 eq). The mixture was stirred at 25° C. for 12 h. The mixture was poured into H₂O (80.0 mL) and extracted with Ethyl acetate (3×40.0 mL). The combined organic layer was washed with saturated NaCl aqueous (2×40.0 mL), saturated Na₂CO₃ aqueous (2×40.0 mL), dried over Na₂SO₄ and concentrated under reduced pressure to get the title compound (12.0 g, crude) as colorless oil. (ESI⁺) m/z: 244.18 (M+H)⁺, (C₁₃H₂₅NO₃).

B. 1-Methyl-2-((1r,4r)-4-methylcyclohexyl)-1H-imidazole: To a solution of (1r,4r)-N-(2,2-dimethoxyethyl)-N,4-dimethylcyclohexane-1-carboxamide (6.00 g, 24.6 mmol, 1.00 eq), NH₄OAc (19.0 g, 246 mmol, 10.0 eq) in AcOH (120 mL) under N₂. The mixture was stirred at 110° C. for 12 h under N₂. The mixture was poured into H₂O (100 mL) and extracted with Ethyl acetate (3×80.0 mL). The combined organic layer was washed with saturated NaCl aqueous (3×80.0 mL), dried over Na₂SO₄ and concentrated under reduced pressure to get residue. The residue was purified preparative-HPLC (column: Phenomenex luna C18 (250×70 m m, 10 um) and gradient of 25%-55% acetonitrile in water containing 0.05% NH₄HCO₃ over 20 min at a flow rate of 25 mL/min) to give the title compound (1.20 g, 3.36 mmol, 13.6% yield, 84.5% purity in LCMS at 220 nm) as yellow solid. ¹H NMR: (400 MHz, DMSO-d₆) δ 6.93 (s, 1H), 6.79 (s, 1H), 3.59 (s, 3H), 2.59-2.53 (m, 1H), 1.88-1.84 (m, 4H), 1.81-1.72 (m, 2H), 1.71-1.68 (m, 1H), 1.06-1.00 (m, 2H), 0.93-0.92 (m, 3H). (ESI⁺) m/z: 179.15 (M+H)⁺, (C₁₁H₁₁N₂).

C. 4, 5-Dibromo-1-methyl-2-((1r,4r)-4-methylcyclohexyl)-1H-imidazole: To a solution of 1-methyl-2-((1r,4r)-4-methylcyclohexyl)-1H-imidazole (300 mg, 1.68 mmol, 1.00 eq) in DMF (3.00 mL) was added NBS (688 mg, 3.87 mmol, 2.30 eq) dropwise at 0° C. The mixture was stirred at 25° C. for 2 h. The mixture was poured into 5.0% K₂CO₃ (aq, 5.00 mL), 10% Na₂S₂O₃ (aq, 10.0 mL) and extracted with Ethyl acetate (3×10 mL). The combined organic layer was washed with saturated NaCl aqueous (2×10.0 mL), dried over Na₂SO₄ and concentrated under reduced pressure to get residue. The crude product was purified by preparative-TLC (Petroleum ether:Ethyl acetate=10:1, R_{f}=0.25) to give the title compound (200 mg, 594 μmol, 35.3% yield, 99.8% purity in LCMS at 220 nm) as white solid. ¹H NMR: (400 MHz, DMSO-d₆) δ 3.55 (s, 3H), 2.59-2.53 (m, 1H), 1.87-1.80 (m, 4H), 1.70-1.66 (m, 2H), 1.55-1.54 (m, 1H), 1.03-1.00 (m, 2H), 0.92-0.91 (m, 3H). (ESI⁺) m/z: 336.7 (M+H)⁺, (C₁₁H₁₆Br₂N₂).

D. 4-Bromo-1-methyl-2-((1r,4r)-4-methylcyclohexyl)-1H-imidazole: To a solution of 4,5-dibromo-1-methyl-2-((1r,4r)-4-methylcyclohexyl)-1H-imidazole (480 mg, 1.43 mmol, 1.00 eq) in THF (10.0 mL) was added n-BuLi (2.50 M, 685 μL, 1.20 eq) dropwise under N₂. The mixture was stirred at −70° C. for 1 h under N₂. The mixture was poured into H₂O (10.0 mL) and extracted with Ethyl acetate (3×5.00 mL). The combined organic layer was washed with saturated NaCl aqueous (2×10.0 mL), dried over Na₂SO₄ and concentrated under reduced pressure to get residue. The crude product was purified by preparative-TLC (Petroleum ether:Ethyl acetate=5:1, R_{f}=0.30) to give the title compound (220 mg, 855 μmol, 59.8% yield, 100% purity in LCMS at 220 nm) as white solid. ¹H NMR: (400 MHz, DMSO-d₆) δ 6.71 (s, 1H), 3.56 (s, 3H), 2.56-2.50 (m, 1H), 1.86-1.80 (m, 4H), 1.73-1.69 (m, 2H), 1.64-1.47 (m, 1H), 1.03-1.01 (m, 2H), 0.93-0.91 (m, 3H). (ESI⁺) m/z: 258.9 (M+H)⁺, (C₁₁H₁₇BrN₂).

E. 3-(5-(1-Methyl-2-((1r,4r)-4-methylcyclohexyl)-1H-imidazol-4-yl)-1-oxoisoindolin-2-yl)piperidine-2,6-dione: To a solution of 4-bromo-1-methyl-2-((1r,4r)-4-methylcyclohexyl)-1H-imidazole (220 mg, 855 μmol, 1.00 eq), 3-(1-oxo-5-(4,4,5,5-tetramethyl-1,3,2-dioxaborolan-2-yl)isoindolin-2-yl)piperidine-2,6-dione (665 mg, 1.80 mmol, 2.10 eq) and K₃PO₄ (363 mg, 1.71 mmol, 2.00 eq) in dioxane (5.00 mL) and H₂O (0.25 mL) was added Ru-Phos-Pd-G₃ (143 mg, 171 μmol, 0.20 eq) under N₂. The mixture was stirred at 100° C. for 2 h under N₂. The mixture was filtered to collected liquid and concentrated under reduced pressure to get residue. The crude product was purified by preparative-HPLC (using a Phenomenex luna C18 (150 mm×25 mm 10 μm) and gradient of 13-43% acetonitrile in water containing 0.5% FA over 14 min at a flow rate of 25 mL/min) to give the title compound (110 mg, 260 μmol, 30.4% yield, 98.7% purity in HPLC at 220 nm) as white solid. 1H NMR: (400 MHz, DMSO-d₆) δ 11.0 (s, 1H), 8.17 (s, 1H), 8.06 (s, 1H), 7.91-7.85 (m, 2H), 5.16-5.11 (m, 1H), 4.55-4.38 (m, 2H), 3.83 (s, 3H), 2.96-2.93 (m, 2H), 2.63-2.59 (m, 1H), 2.45-2.44 (m, 1H), 2.01-2.94 (m, 3H), 1.83-1.80 (m, 4H), 1.73-1.70 (m, 1H), 1.13-1.09 (m, 2H), 0.95-0.93 (m, 3H). (ESI⁺) m/z: 421.2 (M+H)⁺, (C₂₄H₂₈N₄O₃).

### Example 716

### Synthesis of 3-(5-(2-((1R,5S,6s)-3-oxabicyclo[3.1.0]hexan-6-yl)-1-methyl-1H-imidazol-4-yl)-1-oxoisoindolin-2-yl)piperidine-2,6-dione

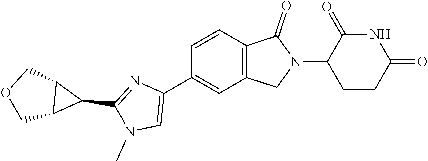 [SMILES: Cn1cc(-c2ccc3c(c2)CN(C2CCC(=O)NC2=O)C3=O)nc1[C@H]1[C@@H]2COC[C@@H]21]

A. tert-Butyl (1R,5S,6r)-3-oxabicyclo[3.1.0]hexane-6-carbonyl chloride: A mixture of (1S,5R)-3-oxabicyclo[3.1.0]hexane-6-carboxylic acid (300 mg, 2.34 mmol, 1.00 eq) in DCM (2.00 mL) was added SOCl₂ (1.60 M, 2.20 mL, 1.50 eq) under N₂, then the reaction mixture was stirred at 25° C. for 1 h. The reaction mixture was concentrated to give the title compound (300 mg, 2.05 mmol, 87.4% yield) as yellow oil. (ESI⁺) m/z: 147.2 (M+H)⁺, (C₆H₇C₁₀₂).

B. tert-Butyl 4-(5-(N-((1R,5S,6r)-3-oxabicyclo[3.1.0]hexane-6-carbonyl)-N-methylglycyl)-1-oxoisoindolin-2-yl)-5-amino-5-oxopentanoate: A mixture of TEA (237 mg, 2.35 mmol, 4.00 eq) and tert-butyl 5-amino-4-(5-(methylglycyl)-1-oxoisoindolin-2-yl)-5-oxopentanoate (250 mg, 586 μmol, 1.00 eq, HCl) in DCM (3.00 mL) was cooled to 0° C., then (1R,5S,6r)-3-oxabicyclo[3.1.0]hexane-6-carboxylic acid (258 mg, 1.76 mmol, 3.00 eq) was added to the reaction mixture, the reaction mixture was stirred at 0° C. for 1 h. The reaction mixture was poured into 10.0 mL H₂O, extracted with EtOAc (3×15.0 ml), dried by Na₂SO₄, concentrated to give the title compound (150 mg, 300 μmol, 51.1% yield) as yellow oil. (ESI⁺) m/z: 500.1 (M+H)⁺, (C₂₆H₃₃N₃O₇).

C. 4-(5-(2-((1R,5S,6r)-3-Oxabicyclo[3.1.0]hexan-6-yl)-1-methyl-1H-imidazol-4-yl)-1-oxoisoindolin-2-yl)-5-amino-5-oxopentanoic acid: A mixture of tert-butyl 5-amino-4-[5-[1-methyl-2-[(1R,5S)-3-oxabicyclo[3.1.0]hexan-6-yl]imidazol-4-yl]-1-oxoisoindolin-2-yl]-5-oxo-pentanoate (200 mg, 416 μmol, 1.00 eq) and TsOH (716 mg, 4.16 mmol, 10.0 eq) in ACN (5.00 mL) was stirred at 100° C. for 12 h. The reaction mixture was concentrated to give residue. The residue was purified by preparative-TLC (SiO₂, Dichloromethane:Methanol=10:1; TLC, Dichloromethane:Methanol=10:1, R_{f}=0.40) to give the title compound (100 mg, 234 μmol, 56.6% yield) as yellow solid. (ESI⁺) m/z: 425.2 (M+H)⁺, (C₂₂H₂₄N₄O₅).

D. 3-(5-(2-((1R,5S,6s)-3-Oxabicyclo[3.1.0]hexan-6-yl)-1-methyl-1H-imidazol-4-yl)-1-oxoisoindolin-2-yl)piperidine-2,6-dione): A mixture of 5-amino-4-[5-[1-methyl-2-[(1R,5S)-3-oxabicyclo[3.1.0]hexan-6-yl]imidazol-4-yl]-1-oxo-isoindolin-2-yl]-5-oxo-pentanoic acid (100 mg, 235 μmol, 1.00 eq) and CDI (152 mg, 942 μmol, 4.00 eq) in ACN (3.00 mL) was stirred at 100° C. for 1 h. Then the reaction mixture was concentrated in vacuum to get a residue. The residue was purified by preparative-TLC (Dichloromethane:Methanol=10:1; TLC, Dichloromethane:Methanol=10:1, R_{f}=0.30) to give the title compound (13.8 mg, 33.7 μmol, 45.6% yield, 98.9% purity in HPLC at 220 nm) as a white solid. ¹H NMR: (400 MHz, DMSO-d₆) δ 10.9 (s, 1H), 7.94 (s, 1H), 7.87-7.78 (m, 3H), 5.14-5.10 (m, 1H), 4.53-4.48 (m, 2H), 4.40-4.39 (m, 2H), 3.97 (s, 3H), 3.78-3.72 (m, 2H), 2.95-2.92 (m, 1H), 2.63-2.50 (m, 2H), 2.44-2.39 (m, 2H), 2.03-1.94 (m, 2H). (ESI⁺) m/z: 407.1 (M+H)⁺, (C₂₂H₂₂N₄O₄).

### Example 717

### Synthesis of 3-(5-(5-(4-chlorophenyl)-1-methyl-1H-pyrazol-3-yl)-1-oxoisoindolin-2-yl)piperidine-2,6-dione

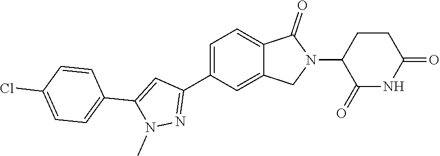 [SMILES: Cn1nc(-c2ccc3c(c2)CN(C2CCC(=O)NC2=O)C3=O)cc1-c1ccc(Cl)cc1]

A. 5-(4-Chlorophenyl)-3-iodo-1H-pyrazole: To a solution of 5-(4-chlorophenyl)-1H-pyrazol-3-amine (5.00 g, 25.8 mmol, 1.00 eq) and TsOH (11.1 g, 64.5 mmol, 2.50 eq) in ACN (250 mL) was added dropwise a solution of NaNO₂ (4.45 g, 64.5 mmol, 2.50 eq) in H₂O (25.0 mL) at 0° C. The reaction mixture was stirred at 0° C. for 0.5 h. Then to the reaction mixture was added dropwise a solution of NaI (19.3 g, 129 mmol, 5.00 eq) in H₂O (25.0 mL) at 0° C. The reaction mixture was stirred at 25° C. for 12 h. The reaction mixture was poured into H₂O (500 mL) at 0° C., and then extracted with DCM (3×500 mL). The combined organic layers were washed by solution of Na₂S₂O₃ (3×300 mL), dried over Na₂SO₄, filtered and concentrated under reduced pressure to give a residue. The residue was purified by column chromatography (SiO₂, Petroleum ether:Ethyl acetate=100:1 to 20:1; TLC, Petroleum ether:Ethyl acetate=5:1, R_{f}=0.30) to give the title compound (3.50 g, 11.3 mmol, 43.8% yield) as yellow solid. ¹H NMR: (400 MHz, CDCl₃) δ 7.56-7.54 (m, 2H), 7.42-7.40 (m, 2H), 6.70 (s, 1H). m/z: 204.8 (M+H)⁺, (C₉H₆ClIN₂).

B. 5-(4-Chlorophenyl)-3-iodo-1-methyl-1H-pyrazole: To a solution of 5-(4-chlorophenyl)-3-iodo-1H-pyrazole (3.00 g, 9.85 mmol, 1.00 eq) and K₂CO₃ (2.72 g, 19.7 mmol, 2.00 eq) in DMF (30.0 mL) was added Mel (6.99 g, 49.2 mmol, 5.00 eq) at 25° C. The reaction mixture was stirred at 25° C. for 4 h. The reaction mixture was poured into H₂O (100 mL), and then extracted with ethyl acetate (3×50.0 mL), dried over Na₂SO₄, filtered and concentrated under reduced pressure to give a residue. The residue was purified by column chromatography (SiO₂, Petroleum ether:Ethyl acetate=100:1 to 50:1; TLC, Petroleum ether:Ethyl acetate=8:1, R_{f}=0.40) and preparative-HPLC (using a Welch Xtimate C18 (200 mm×40 mm×10 μm) and gradient of 40-70% acetonitrile in water containing 0.05% TFA over 15 min at a flow rate of 25.0 mL/min) to give the title compound (0.50 g, 1.57 mmol, 15.9% yield) as white solid. ¹H NMR: (400 MHz, CDCl₃) δ 7.45 (d, J=8.8 Hz, 2H), 7.32 (d, J=8.4 Hz, 2H), 6.45 (s, 1H), 3.87 (s, 3H). m/z: 318.7 (M+H)⁺, (C₁₀H₈ClIN₂).

C. 3-(5-(5-(4-Chlorophenyl)-1-methyl-1H-pyrazol-3-yl)-1-oxoisoindolin-2-yl)piperidine-2,6-dione: To a solution of 5-(4-chlorophenyl)-3-iodo-1-methyl-1H-pyrazole (300 mg, 941 μmol, 1.00 eq) and 3-[1-oxo-5-(4,4,5,5-tetramethyl-1,3,2-dioxaborolan-2-yl)isoindolin-2-yl]piperidine-2,6-dione (871 mg, 2.35 mmol, 2.50 eq) in dioxane (5.00 mL) and H₂O (0.25 mL) was added Ru-Phos-Pd-G₃ (157 mg, 188 μmol, 0.20 eq), K₃PO₄ (399 mg, 1.88 mmol, 2.00 eq) under N₂. The reaction mixture was stirred at 100° C. for 6 h under N₂. The reaction mixture was concentrated in vacuum to give residue. The residue was purified by preparative-TLC (SiO₂, Dichloromethane:Methanol=20:1; TLC, Dichloromethane:Methanol=20:1, R_{f}=0.40) and preparative-HPLC (using a Welch Xtimate C18 (150 mm×25 mm×5 μm) and gradient of 35-65% acetonitrile in water containing 0.05% TFA over 15 min at a flow rate of 25.0 mL/min) to give the title compound (17.9 mg, 41.1 μmol, 4.37% yield, 99.8% purity in HPLC at 220 nm) as white solid. ¹H NMR: (400 MHz, DMSO-d₆) δ 11.0 (s, 1H), 8.07 (s, 1H), 7.99 (d, J=9.2 Hz, 1H), 7.77 (d, J=8.0 Hz, 1H), 7.67-7.62 (m, 4H), 7.07 (s, 1H), 5.16-5.11 (m, 1H), 4.54-4.36 (m, 2H), 3.93 (s, 3H), 2.97-2.90 (m, 1H), 2.63-2.59 (m, 1H), 2.45-2.40 (m, 1H), 2.04-2.02 (m, 1H). (ESI⁺) m/z: 435.1 (M+H)⁺, (C₂₃H₁₉CIN₄O₃).

### Example 718

### Synthesis of 3-(5-(2-(bicyclo[2.2.2]octan-1-yl)-1-methyl-1H-imidazol-4-yl)-1-oxoisoindolin-2-yl)piperidine-2,6-dione

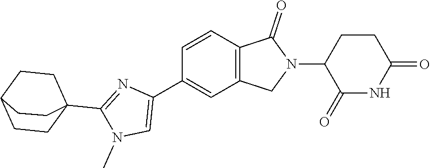 [SMILES: Cn1cc(-c2ccc3c(c2)CN(C2CCC(=O)NC2=O)C3=O)nc1C12CCC(CC1)CC2]

A. Bicyclo[2.2.2]octane-1-carbonyl chloride: To a solution of bicyclo[2.2.2]octane-1-carboxylic acid (200 mg, 1.30 mmol, 1.00 eq) in DCM (2.00 mL) was added SOCl₂ (1.6 M, 2.03 mL, 2.50 eq) at 0° C. Then mixture was stirred at 0° C. for 2 h. The reaction mixture was concentrated under reduced pressure to give the title compound (200 mg, 1.16 mmol, 89.3% yield in LCMS at 220 nm) as light yellow oil. (ESI⁺) m/z: 173.1 (M+H)⁺, (C₉H₁₃Cl₀).

B. tert-Butyl 5-amino-4-(5-(N-(bicyclo[2.2.2]octane-1-carbonyl)-N-methylglycyl)-1-oxoisoindolin-2-yl)-5-oxopentanoate: To a solution of tert-butyl 5-amino-4-(5-(methylglycyl)-1-oxoisoindolin-2-yl)-5-oxopentanoate (165 mg, 387 μmol, 1.00 eq, HCl) and TEA (392 mg, 3.87 mmol, 539 μL, 10.0 eq) in DCM (2.00 mL) was added bicyclo[2.2.2]octane-1-carbonyl chloride (200 mg, 1.16 mmol, 3.00 eq) in DCM (2.00 mL) at 0° C. The mixture was stirred at 0° C. for 2 h. The reaction mixture was poured into H₂O (30.0 mL) and extracted with Ethyl acetate (3×50.0 mL). The combined organic layer was washed with brine (3×50.0 mL), dried over Na₂SO₄, filtered and concentrated. The residue was purified by TLC (Dichloromethane:Methanol=20:1,R_{f}=0.40) to give the title compound (160 mg, 273 μmol, 70.7% yield, 90.0% purity in LCMS at 220 nm) as light yellow oil. (ESI⁺) m/z: 526.2 (M+H)⁺, (C₂₉H₃₉N₃O₆).

C. tert-Butyl 5-amino-4-(5-(2-(bicyclo[2.2.2]octan-1-yl)-1-methyl-1H-imidazol-4-yl)-1-oxoisoindolin-2-yl)-5-oxopentanoate: To a solution of tert-butyl 5-amino-4-(5-(N-(bicyclo[2.2.2]octane-1-carbonyl)-N-methylglycyl)-1-oxoisoindolin-2-yl)-5-oxopentanoate (190 mg, 361 μmol, 1.00 eq) and NH₄OAc (195. mg, 2.53 mmol, 7.00 eq) in HCONH₂ (10.0 mL) under N₂. The mixture was stirred at 100° C. for 16 h under N₂. The reaction mixture was poured into H₂O (30.0 mL) and extracted with Ethyl acetate (3×50.0 mL). The combined organic layer was washed with brine (3×50.0 mL), dried over Na₂SO₄, filtered and concentrated. The residue was purified by TLC (Dichloromethane:Methanol=15:1,R_{f}=0.30) to give the title compound (130 mg, 233 μmol, 64.6% yield, 91.1% purity in LCMS at 220 nm) as light yellow oil. (ESI⁺) m/z: 507.2 (M+H)⁺, (C₂₉H₃₈N₄O₄).

D. 3-(5-(2-(Bicyclo[2.2.2]octan-1-yl)-1-methyl-1H-imidazol-4-yl)-1-oxoisoindolin-2-yl)piperidine-2,6-dione: To a solution of tert-butyl 5-amino-4-(5-(2-(bicyclo[2.2.2]octan-1-yl)-1-methyl-1H-imidazol-4-yl)-1-oxoisoindolin-2-yl)-5-oxopentanoate (130 mg, 233 μmol, 1.00 eq) in ACN (6.00 mL) was added TsOH (120 mg, 700 μmol, 3.00 eq) under N₂. The mixture was stirred at 80° C. for 4 h under N₂. The reaction mixture was concentrated under reduced pressure to get a residue at 45° C. The residue was purified by preparative-HPLC (using a Phenomenex luna C18 (150×25 mm×5 μm) and gradient of 8.0%-38.0% acetonitrile in water containing 0.05% TFA over 10 min at a flow rate of 25 mL/min to give the title compound (53.7 mg, 123 μmol, 52.7% yield, 99.2% purity in HPLC at 220 nm) as white solid. ¹H NMR: (400 MHz, DMSO-d₆) δ 11.00-10.97 (m, 1H), 8.03-7.95 (m, 2H), 7.91-7.81 (m, 2H), 5.15-5.12 (m, 1H), 4.53-4.36 (m, 2H), 3.94-3.88 (m, 3H), 2.95-2.91 (m, 1H), 2.63-2.59 (m, 1H), 2.45-2.40 (m, 1H), 2.03-2.01 (m, 7H), 1.69-1.68 (m, 7H). (ESI⁺) m/z: 433.2 (M+H)⁺, (C₂₅H₂₈N₄O₃).

### Example 719

### Synthesis of 3-(5-(1-methyl-2-(quinuclidin-4-yl)-1H-imidazol-4-yl)-1-oxoisoindolin-2-yl)piperidine-2,6-dione

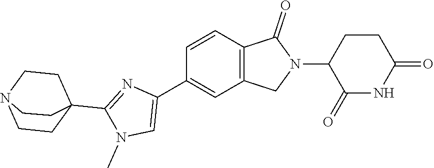 [SMILES: Cn1cc(-c2ccc3c(c2)CN(C2CCC(=O)NC2=O)C3=O)nc1C12CCN(CC1)CC2]

A. Quinuclidine-4-carbonyl chloride: To a solution of quinuclidine-4-carboxylic acid (200 mg, 1.04 mmol, 1.00 eq, HCl) in DCM (2.00 mL) was added SOCl₂ (1.6 M, 2.61 mL, 4.00 eq) at 0° C. Then the mixture was stirred at 25° C. for 12 h. The reaction mixture was concentrated in vacuum to give the title compound (180 mg, crude) as an off-white solid. (ESI⁺) m/z: 170.1 (M−Cl+OMe+H)⁺, (C₈H₁₂NOCl).

B. tert-Butyl 5-amino-4-(5-(N-methyl-N-(quinuclidine-4-carbonyl)glycyl)-1-oxoisoindolin-2-yl)-5-oxopentanoate: To a solution of tert-butyl 5-amino-4-[5-[2-(methylamino)acetyl]-1-oxo-isoindolin-2-yl]-5-oxo-pentanoate (100 mg, 256 μmol, 1.00 eq) and quinuclidine-4-carbonyl chloride (133 mg, 770 μmol, 3.00 eq) in DCM (1.00 mL) was added TEA (259 mg, 2.57 mmol, 357 μL, 10.0 eq) at 0° C. Then the reaction mixture was stirred at 0° C. for 0.5 h. The reaction mixture was diluted with H₂O (3.00 mL) and extracted with DCM (3×5.00 mL). The combined organic layers were washed with brine, dried over Na₂SO₄ and filtered. The filtrate was concentrated in vacuum to give the title compound (130 mg, crude) as yellow oil. (ESI⁺) m/z: 527.1 (M+H)⁺, (C₂₈H₃₈N₄O₆).

C. tert-Butyl 5-amino-4-(5-(1-methyl-2-((1r,4r)-4-(trifluoromethyl)cyclohexyl)-1H-imidazol-4-yl)-1-oxoisoindolin-2-yl)-5-oxopentanoate: To a solution of tert-butyl 5-amino-4-[5-[2-[methyl(quinuclidine-4-carbonyl)amino]acetyl]-1-oxo-isoindolin-2-yl]-5-oxo-pentanoate (130 mg, 246 μmol, 1.00 eq) in HCONH₂ (10.0 mL) was added NH₄OAc (95.1 mg, 1.23 mmol, 5.00 eq). The mixture was stirred at 100° C. for 16 h. Then the reaction mixture was purified by prep-HPLC (0.1% FA condition) to give the title compound (60.0 mg, 115 μmol, 34.5% yield, 97.6% purity in HPLC at 220 nm) as a yellow solid. (ESI⁺) m/z: 508.2 (M+H)⁺, (C₂₈H₃₇N₅O₄).

D. 3-(5-(1-Methyl-2-(quinuclidin-4-yl)-1H-imidazol-4-yl)-1-oxoisoindolin-2-yl)piperidine-2,6-dione: To a solution of tert-butyl 5-amino-4-[5-(1-methyl-2-quinuclidin-4-yl-imidazol-4-yl)-1-oxo-isoindolin-2-yl]-5-oxo-pentanoate (60.0 mg, 115 μmol, 1.00 eq) in ACN (2.00 mL) was added TsOH (79.4 mg, 461 μmol, 4.00 eq). The mixture was stirred at 80° C. for 16 h. The reaction mixture was concentrated in vacuum to get a residue. The residue was purified by preparative-HPLC (using a Waters xbridge (150 mm×25 mm 10 μm) and gradient of 1-30% acetonitrile in water containing 0.5% NH₄HCO₃ over 10 min at a flow rate of 25.0 mL/min) to give the title compound (11.9 mg, 27.2 μmol, 23.2% yield, 99.1% purity in HPLC at 220 nm) as an off-white solid. ¹H NMR: (400 MHz, DMSO-d₆) δ 10.9 (s, 1H), 7.89 (s, 1H), 7.82 (d, J=8.0 Hz, 1H), 7.69-7.63 (m, 2H), 5.10 (dd, J=13.6, 5.2 Hz, 1H), 4.39 (dd, J=53.2, 17.2 Hz, 2H), 3.78 (s, 3H), 2.97-2.90 (m, 1H), 2.89-2.81 (m, 6H), 2.64-2.57 (m, 1H), 2.46-2.35 (m, 1H), 2.05-1.97 (m, 1H), 1.95-1.86 (m, 6H). (ESI⁺) m/z: 434.0 (M+H)⁺, (C₂₄H₂₇N₅O₃).

### Example 720

### Synthesis of 3-(5-(2-ethyl-5-phenyloxazol-4-yl)-1-oxoisoindolin-2-yl) piperidine-2, 6-dione

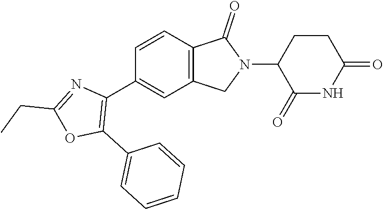 [SMILES: CCc1nc(-c2ccc3c(c2)CN(C2CCC(=O)NC2=O)C3=O)c(-c2ccccc2)o1]

A. 2-Ethyl-5-phenyloxazole: To a solution of acetophenone (2.04 g, 16.97 mmol, 1.98 mL, 0.50 eq) in DMSO (30.0 mL) was added 12 (8.61 g, 33.9 mmol, 6.84 mL, 1.00 eq). Then the mixture was stirred at 110° C. for 0.5 h. Then 2-aminobutanoic acid (3.50 g, 33.9 mmol, 1.00 eq) was added into the mixture. Then the reaction was stirred at 110° C. for 0.5 h. The mixture was poured into water (200 mL) extracted with ethyl acetate (3×100 mL) to collect the organic layer, the combined organic layers were washed with saturated aqueous Na₂S₂O₃ (3×100 mL). Then the organic layers were dried over Na₂SO₄, filtered and the filtrate was concentrated under vacuum to get a residue. The residue was purified by column chromatography (SiO₂, Petroleum ether/Ethyl acetate=100/1 to 1/1, TLC:Petroleum ether/Ethyl acetate=5/1, R_{f}=0.30) to give the title compound (1.00 g, 5.37 mmol, 15.8% yield, 93.0% purity in LCMS at 220 nm) as yellow oil. m/z: 174.0 (M+H)⁺, (C₁₁H₁₁NO).

B. 4-Bromo-2-ethyl-5-phenyloxazole: To a solution of 2-ethyl-5-phenyloxazole (1.00 g, 5.37 mmol, 1.00 eq) in ACN (12.0 mL) was added NBS (1.24 g, 6.98 mmol, 1.30 eq) at 25° C. Then the mixture was stirred at 70° C. for 3 h. The mixture was concentrated under reduce pressure to get residue. The residue was purified by preparative-TLC (Petroleum ether Ethyl acetate=5:1; R_{f}=0.40) to give the title compound (650 mg, 2.49 mmol, 46.4% yield, 96.7% purity in LCMS at 220 nm) as yellow oil. m/z: 251.9 (M+H)⁺, (C₁₁H₁₀BrNO).

C. 3-(5-(2-Ethyl-5-phenyloxazol-4-yl)-1-oxoisoindolin-2-yl) piperidine-2, 6-dione: To a solution of 4-bromo-2-ethyl-5-phenyloxazole (300 mg, 1.19 mmol, 1.00 eq), 3-(1-oxo-5-(4,4,5,5-tetramethyl-1,3,2-dioxaborolan-2-yl)isoindolin-2-yl)piperidine-2,6-dione (881 mg, 2.38 mmol, 2.00 eq) and K₃PO₄ (758 mg, 3.57 mmol, 3.00 eq) in dioxane (6.00 mL) and H₂O (0.30 mL) was added Ru-Phos-Pd-G₃ (99.5 mg, 119 μmol, 0.10 eq) under N₂. The mixture was stirred at 100 SC for 2 h under N₂. The reaction mixture was concentrated under reduced pressure to get a residue at 45° C. The residue was purified by preparative-HPLC (using a Welch Xtimate C18 (150 mm×25 mm×10 μm) and gradient of 29-59% acetonitrile in water containing 0.05% TFA over 15 min at a flow rate of (25.0 mL/min) to give the title compound (229 mg, 542 μmol, 45.5% yield, 98.1% purity in HPLC at 220 nm). ¹H NMR: (400 MHz, DMSO-d₆) δ 11.0 (s, 1H), 7.82 (s, 1H), 7.75-7.69 (m, 2H), 7.57-7.55 (m, 2H), 7.47-7.44 (m, 3H), 5.15-5.10 (m, 1H), 4.50-4.32 (m, 2H), 2.91-2.85 (m, 3H), 2.62-2.58 (m, 1H), 2.41-2.38 (m, 1H), 2.04-2.02 (m, 1H), 1.36-1.32 (m, 3H). (ESI⁺) m/z: 416.1 (M+H)⁺, (C₂₄H₂IN₃O₄).

### Examples 721-725

The compounds of Example 721-725 were prepared according to Scheme 5 as described in Examples 555-587.

| | | MW + 1 | Step 1 |
|---|---|---|---|
| Example | Structure | (Observed) | Conditions |
| 721 | 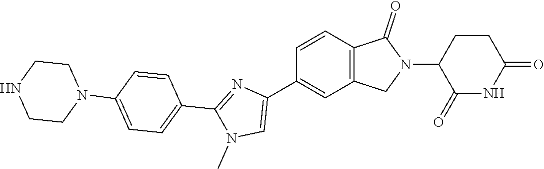 [SMILES: Cn1cc(-c2ccc3c(c2)CN(C2CCC(=O)NC2=O)C3=O)nc1-c1ccc(N2CCNCC2)cc1] | 485.3 | 2 |
| 722 | 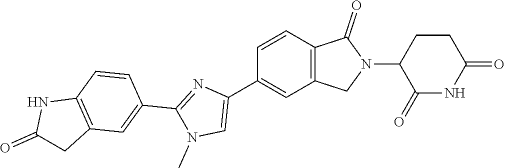 [SMILES: Cn1cc(-c2ccc3c(c2)CN(C2CCC(=O)NC2=O)C3=O)nc1-c1ccc2c(c1)CC(=O)N2] | 456.3 | 2 |
| 723 | 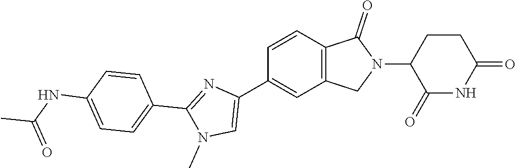 [SMILES: CC(=O)Nc1ccc(-c2nc(-c3ccc4c(c3)CN(C3CCC(=O)NC3=O)C4=O)cn2C)cc1] | 458.3 | 1 |
| 724 | 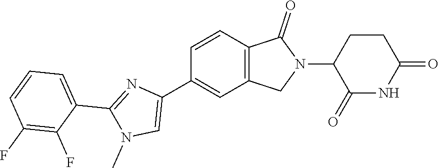 [SMILES: Cn1cc(-c2ccc3c(c2)CN(C2CCC(=O)NC2=O)C3=O)nc1-c1cccc(F)c1F] | 437.2 | 2 |
| 725 | 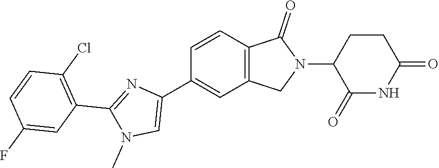 [SMILES: Cn1cc(-c2ccc3c(c2)CN(C2CCC(=O)NC2=O)C3=O)nc1-c1cc(F)ccc1Cl] | 453.3 | 2 |

### Example 726

### Synthesis of 3-(5-(2-isopropyl-1-methyl-5-phenyl-1H-imidazol-4-yl)-1-oxoisoindolin-2-yl)piperidine-2,6-dione

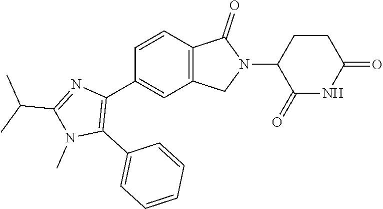 [SMILES: CC(C)c1nc(-c2ccc3c(c2)CN(C2CCC(=O)NC2=O)C3=O)c(-c2ccccc2)n1C]

A. N-(2,2-Dimethoxyethyl)-N-methylisobutyrimidamide: To a mixture of 2,2-dimethoxy-N-methyl-ethanamine (5.00 g, 41.9 mmol, 5.39 mL, 1.00 eq) and CuCl (5.19 g, 52.4 mmol, 1.25 mL, 1.25 eq) was added 2-methylpropanenitrile (3.96 g, 57.3 mmol, 1.37 eq). The reaction mixture was stirred at 85° C. for 12 h under N₂. Then reaction mixture was concentrated in vacuum to give the title compound (8.00 g, crude) as brown oil. (ESI⁺) m/z: 189.0 (M+H)⁺, (C₉H₂₀N₂₀₂).

B. 2-Isopropyl-1-methyl-1H-imidazole: To a solution of N-(2,2-dimethoxyethyl)-N,2-dimethyl-propanamidine (8.00 g, 42.49 mmol, 1.00 eq) in MeOH (40.0 mL) was added HCl (12.0 M, 8.00 mL, 2.26 eq). The mixture was stirred at 80° C. for 4 h. The reaction mixture was concentrated in vacuum and the residue was diluted with 50.0 mL of water. The mixture was basified with 15.0 mL of 50% NaOH solution to pH=12 keeping the temperature below 20° C. Then 50.0 mL of Dichloromethane/MeOH (10/1) was added, the mixture was stirred for 5 min and filtered. The filter cake was washed with Dichloromethane/MeOH (10/1) (2×50.0 mL). The combined organic layers were dried over Na₂SO₄ and filtered. The filtrate was concentrated in vacuum to get a residue. The residue was purified by column chromatography (SiO₂, Dichloromethane:Methanol=100:1 to 50:1, R_{f}=0.45 (Dichloromethane:Methanol=5:1)) to give the title compound (1.50 g, 10.7 mmol, 16.7% yield for two steps, 89.0% purity in HPLC at 220 nm) as brown oil. (ESI⁺) m/z: 125.0 (M+H)⁺, (C₇H₁₂N₂).

C. 2-Isopropyl-1-methyl-5-phenyl-1H-imidazole: To a solution of 2-isopropyl-1-methyl-imidazole (1.40 g, 10.0 mmol, 1.00 eq) and bromobenzene (4.73 g, 30.1 mmol, 3.17 mL, 3.00 eq) in DMF (28.0 mL) was added Pd(OAc)₂ (225 mg, 1.00 mmol, 0.10 eq), P(oxole)₃ (465 mg, 2.01 mmol, 0.20 eq) and K₂CO₃ (2.77 g, 20.0 mmol, 2.00 eq). The mixture was stirred at 100° C. for 12 h under N₂. The reaction mixture was filtered and the filtrate was concentrated in vacuum to get a residue. The residue was purified by column chromatography (SiO₂, Dichloromethane:Methanol=100:1 to 10:1, R_{f}=0.40 (Dichloromethane:Methanol=10:1)) to give the title compound (1.00 g, 4.99 mmol, 49.7% yield) as yellow oil. ¹H NMR: (400 MHz, DMSO-d₆) δ 7.48-7.40 (m, 4H), 7.38-7.34 (m, 1H), 6.87 (s, 1H), 3.54 (s, 3H), 3.15-3.06 (m, 1H), 1.24 (d, J=6.8 Hz, 6H), (ESI⁺) m/z: 201.0 (M+H)⁺, (C₁₃H₁₆N₂).

D. 4-Bromo-2-isopropyl-1-methyl-5-phenyl-1H-imidazole: To a solution of 2-isopropyl-1-methyl-5-phenyl-imidazole (1.00 g, 4.99 mmol, 1.00 eq) in ACN (10.0 mL) was added NBS (933 mg, 5.24 mmol, 1.05 eq) at 0° C. The mixture was stirred at 25° C. for 2 h under N₂. The reaction mixture was diluted with 20.0 mL of H₂O and extracted with ethyl acetate (3×20.0 mL). The combined organic layers were washed with brine, dried over Na₂SO₄ and filtered. The filtrate was concentrated in vacuum to get a residue. The residue was purified by column chromatography (SiO₂, Petroleum ether:Ethyl acetate=100:1 to 5:1, R_{f}=0.70 (Petroleum ether:Ethyl acetate=3:1)) to give the title compound (800 mg, 2.51 mmol, 50.2% yield, 87.5% purity in LCMS at 220 nm) as yellow oil. (ESI⁺) m/z: 279.0 (M+H)⁺, (C₁₃H₁₅N₂Br).

E. 3-(5-(2-Isopropyl-1-methyl-5-phenyl-1H-imidazol-4-yl)-1-oxoisoindolin-2-yl)piperidine-2,6-dione: To a solution of 4-bromo-2-isopropyl-1-methyl-5-phenyl-imidazole (400 mg, 1.25 mmol, 1.00 eq) and 3-[1-oxo-5-(4,4,5,5-tetramethyl-1,3,2-dioxaborolan-2-yl)isoindolin-2-yl]piperidine-2,6-dione (1.16 g, 3.13 mmol, 2.50 eq) in dioxane (8.00 mL) and H₂O (0.40 mL) was added K₃PO₄ (798 mg, 3.76 mmol, 3.00 eq) and Ru-Phos-Pd-G₃ (209 mg, 250 μmol, 0.20 eq). The mixture was stirred at 100° C. for 2 h under N₂. The reaction mixture was filtered and the filtrate was concentrated in vacuum to get a residue. The residue was purified by column chromatography (SiO₂, Dichloromethane:Methanol=100:1 to 10:1, R_{f}=0.50 (Dichloromethane:Methanol=10:1)) and then by preparative-HPLC (using a Phenomenex Luna C18 (100 mm×30 mm 5 μm) and gradient of 10-40% acetonitrile in water containing 0.5% HCl over 8 min at a flow rate of 25.0 mL/min) to give the title compound (220 mg, 448 μmol, 35.7% yield, 99.5% purity in HPLC at 220 nm) as a white solid. ¹H NMR: (400 MHz, DMSO-d₆) δ 10.9 (s, 1H), 7.58 (s, 1H), 7.55-7.47 (m, 4H), 7.43-7.37 (m, 3H), 5.06 (dd, J=13.2, 4.8 Hz, 1H), 4.27 (dd, J=59.6, 17.2 Hz, 2H), 3.37 (s, 3H), 3.21-3.12 (m, 1H), 2.95-2.84 (m, 1H), 2.61-2.55 (m, 1H), 2.41-2.30 (m, 1H), 2.01-1.92 (m, 1H), 1.32 (d, J=6.8 Hz, 6H). (ESI⁺) m/z: 443.2 (M+H)⁺, (C₂₆H₂₆N₄O₃).

### Example 727

### Synthesis of 3-(5-(2-cyclobutyl-1-methyl-5-phenyl-1H-imidazol-4-yl)-1-oxoisoindolin-2-yl)piperidine-2,6-dione

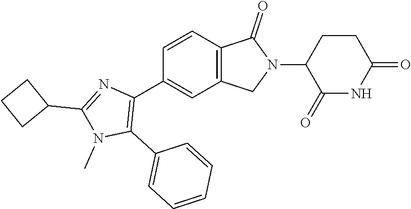 [SMILES: Cn1c(C2CCC2)nc(-c2ccc3c(c2)CN(C2CCC(=O)NC2=O)C3=O)c1-c1ccccc1]

A. N-(2,2-Dimethoxyethyl)-N-methylcyclobutanecarboximidamide: To a solution of cyclobutanecarbonitrile (10 g, 123 mmol, 1.00 eq) and 2,2-dimethoxy-N-methylethan-1-amine (11.7 g, 98.6 mmol, 12.6 mL, 0.80 eq) was added CuCl (15.2 g, 154 mmol, 3.68 mL, 1.25 eq). The mixture was stirred at 85° C. for 12 h. The reaction mixture was concentrated under reduced pressure to give the title compound (20.0 g, crude) as brown oil. (ESI⁺) m/z: 201.2 (M+H)⁺, (C₁₀H₂₀N₂O₂).

B. 2-Cyclobutyl-1-methyl-1H-imidazole: To a solution of N-(2,2-dimethoxyethyl)-N-methylcyclobutanecarboximidamide (20 g, 99.8 mmol, 1.00 eq) in MeOH (40.0 mL) and HCl (24.7 mL) under N₂. The mixture was stirred at 80° C. for 6 h under N₂. The mixture was poured into 50% aq NaOH and extracted with Ethyl acetate (2×300 mL). The combined organic layer was dried over Na₂SO₄ and concentrated under reduced pressure to get residue. The crude product was purified by column chromatography (SiO₂, Dichloromethane:Methanol=500:1 to 20:1, TLC:Dichloromethane:Methanol=20:1,R_{f}=0.20) to give the title compound (4.00 g, 29.2.mmol, 29.2% yield, 99.5% purity in LCMS at 220 nm) as brown oil. (ESI⁺) m/z: 137.1 (M+H)⁺, (C₈H₁₂N₂).

C. 2-Cyclobutyl-1-methyl-5-phenyl-1H-imidazole: To a solution of 2-cyclobutyl-1-methyl-1H-imidazole (1.00 g, 7.34 mmol, 1.00 eq) and bromobenzene (3.46 g, 22.0 mmol, 2.32 mL 3.00 eq) in DMF (15.0 mL) was added P(oxole)₃ (340 mg, 1.47 mmol, 0.20 eq), K₂CO₃ (2.03 g, 14.6 mmol, 2.00 eq) and Pd(OAc)₂ (164 mg, 0.74 mmol, 0.10 eq) under N₂. The mixture was stirred at 100° C. for 12 h under N₂. The reaction mixture was filtered through celite. The reaction mixture was poured into H₂O (60.0 mL) and extracted with Ethyl acetate (3×60 mL). The combined organic layer was washed with brine (3×60 mL), dried over Na₂SO₄, filtered and concentrated. The crude product was purified by column chromatography (SiO₂, Petroleum ether:Ethyl acetate=50:1 to 0:1, TLC:Petroleum ether: Ethyl acetate=3:1,R_{f}=0.30) to give the title compound (2.10 g, 8.52 mmol, 58.0% yield, 86.1% purity in LCMS at 220 nm) as yellow solid. ¹H NMR: (400 MHz, DMSO-d₆) δ 7.46-7.40 (m, 4H), 7.37-7.32 (m, 1H), 6.91 (s, 1H), 3.67-3.63 (m, 1H), 3.45 (s, 3H), 2.36-2.29 (m, 4H), 2.02-1.99 (m, 1H), 1.88-1.82 (m, 1H). (ESI⁺) m/z: 213.0 (M+H)⁺, (C₁₄H₁₆N₂).

D. 4-Bromo-2-cyclobutyl-1-methyl-5-phenyl-1H-imidazole: To a solution of 2-cyclobutyl-1-methyl-5-phenyl-1H-imidazole (750 mg, 3.04 mmol, 1.00 eq) in ACN (10.0 mL) was added NBS (568 mg, 3.19 mmol, 1.06 eq) at 0° C. The mixture was stirred at 0° C. for 1 h. The reaction mixture was poured into H₂O (60.0 mL) and extracted with Ethyl acetate (3×60 mL). The combined organic layer was washed with brine (3×60 mL), dried over Na₂SO₄, filtered and concentrated. The mixture was purified by preparative-TLC (Petroleum ether:Ethyl acetate=3:1,R_{f}=0.50) to give the title compound (720 mg, 2.23 mmol, 73.3% yield, 90.2% purity in LCMS at 220 nm) as yellow solid. (ESI⁺) m/z: 291.0 (M+H)⁺, (C₁₄H₁₅BrN₂).

E. 3-(5-(2-Cyclobutyl-1-methyl-5-phenyl-1H-imidazol-4-yl)-1-oxoisoindolin-2-yl)piperidine-2,6-dione: To a solution of 4-bromo-2-cyclobutyl-1-methyl-5-phenyl-1H-imidazole (300 mg, 927 μmol, 1.00 eq), 3-(1-oxo-5-(4,4,5,5-tetramethyl-1,3,2-dioxaborolan-2-yl)isoindolin-2-yl)piperidine-2,6-dione (858 mg, 2.32 mmol, 2.50 eq) and K₃PO₄ (590 mg, 2.78 mmol, 3.00 eq) in dioxane (6.00 mL) and H₂O (0.30 mL) was added Ru-Phos-Pd-G₃ (77.5 mg, 92.7 μmol, 0.10 eq) under N₂. The mixture was stirred at 100° C. for 2 h under N₂. The reaction mixture was concentrated under reduced pressure to get a residue at 45° C. The residue was purified by preparative-HPLC (using a Phenomenex luna C18 (150 mm×25 mm×10 μm) and gradient of 19-49% acetonitrile in water containing 0.05% TFA over 15 min at a flow rate of 25.0 mL/min) to give the title compound (385 mg, 840 μmol, 90.6% yield, 99.2% purity in HPLC at 220 nm) as white solid. ¹H NMR: (400 MHz, DMSO-d₆) δ 11.0 (s, 1H), 7.76-7.73 (m, 1H), 7.72-7.68 (m, 4H), 7.45-7.42 (m, 3H), 5.12-5.08 (m, 1H), 4.43-4.30 (m, 2H), 4.06-4.00 (m, 1H), 3.47 (s, 3H), 2.92-2.90 (m, 1H), 2.61-2.56 (m, 3H), 2.46-2.42 (m, 3H), 2.14-2.11 (m, 1H), 1.99-1.93 (m, 2H). (ESI⁺) m/z: 455.2 (M+H)⁺, (C₂₇H₂₆N₄O₃).

### Example 728

### Synthesis of 3-(5-(2-isopropyl-1-phenyl-1H-imidazol-4-yl)-1-oxoisoindolin-2-yl)piperidine-2,6-dione

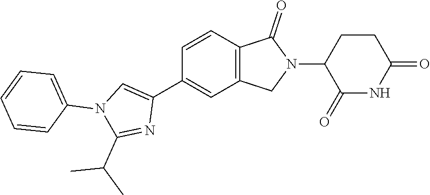 [SMILES: CC(C)c1nc(-c2ccc3c(c2)CN(C2CCC(=O)NC2=O)C3=O)cn1-c1ccccc1]

A. 2-Isopropyl-1-phenyl-1H-imidazole: To a solution of 2-isopropyl-1H-imidazole (5.00 g, 45.4 mmol, 1.00 eq) in MeOH (150 mL) was added phenylboronic acid (4.70 g, 38.5 mmol, 0.85 eq) and Cu₂O (324 mg, 2.27 mmol, 231 μL, 0.0500 eq) at 25° C. under 02. Then the mixture was stirred at 25° C. for 16 h under 02 (15 Psi). The reaction mixture was concentrated under vacuum to give a residue at 45° C. The residue was purified by column chromatography (SiO₂, PE/EtOAc=100/0 to 85/15, TLC: PE/EtOAc=1/1, R_{f}=0.50) to give the title compound (2.68 g, 14.3 mmol, 31.5% yield, 99.4% purity) as a yellow oil. ¹H NMR: (400 MHz, DMSO-d₆) δ 7.56-7.47 (m, 3H), 7.40-7.38 (m, 2H), 7.17 (s, 1H), 6.92 (s, 1H), 2.97-2.90 (m, 1H), 1.11 (d, J=6.8 Hz, 6H). (ESI⁺) m/z: 187.0 (M+H)⁺, (C₁₂H₁₄N₂).

B. 5-Bromo-2-isopropyl-1-phenyl-1H-imidazole: To a solution of 2-isopropyl-1-phenyl-1H-imidazole (1.00 g, 5.34 mmol, 1.00 eq) in ACN (10.0 mL) was added NBS (997 mg, 5.60 mmol, 1.05 eq) at 0° C. Then the mixture was stirred at 25° C. for 1 h. The reaction mixture was diluted with water (50.0 mL) and extracted with EtOAc (2×50.0 mL) to get the organic layers, the organic layers was washed with bine (100 mL) and dried over anhydrous Na₂SO₄, filtered, the filtrate was concentrated under vacuum to give a residue at 45° C. The residue was purified by column chromatography (SiO₂, PE/EtOAc=100/0 to 94.1/5.9, TLC: PE/EtOAc=4/1, R_{f}=0.60) to give the title compound (1.10 g, 4.15 mmol, 77.7% yield, 100% purity in LCMS at 220 nm) as a light yellow solid. ¹H NMR: (400 MHz, DMSO-d₆) δ 7.59-7.56 (m, 3H), 7.38-7.36 (m, 2H), 7.03 (s, 1H), 2.76-2.66 (m, 1H), 1.08 (d, J=6.8 Hz, 6H). (ESI⁺) m/z: 266.7 (M+H)⁺, (C₁₂H₁₃N₂Br).

C. 4,5-Dibromo-2-isopropyl-1-phenyl-1H-imidazole: To a solution of 5-bromo-2-isopropyl-1-phenyl-1H-imidazole (900 mg, 3.39 mmol, 1.00 eq) in DMF (9.00 mL) was added NBS (724 mg, 4.07 mmol, 1.20 eq) at 0° C. Then the mixture was stirred at 25° C. for 1 h. The mixture was diluted with EtOAc (30.0 mL) and washed with 5% K₂CO₃ (aq, 30.0 mL), 10% Na₂S₂O₃ (aq, 40.0 mL). The combined organic layer was washed with saturated NaCl aqueous (2×30.0 mL), dried over Na₂SO₄ and concentrated under reduced pressure to give the title compound (1.10 g, 2.78 mmol, 82.0% yield, 87.1% purity in LCMS at 220 nm) as a light yellow solid. ¹H NMR: (400 MHz, DMSO-d₆) δ 7.63-7.59 (m, 3H), 7.46-7.44 (m, 2H), 2.74-2.67 (m, 1H), 1.08 (d, J=6.8 Hz, 6H). (ESI⁺) m/z: 344.6 (M+H)⁺, (C₁₂H₁₂N₂Br₂).

D. 4-Bromo-2-isopropyl-1-phenyl-1H-imidazole: To a solution of 4,5-dibromo-2-isopropyl-1-phenyl-1H-imidazole (1.10 g, 2.78 mmol, 1.00 eq) in THF (22.0 mL) was added n-BuLi (2.50 M, 1.67 mL, 1.50 eq) at −75° C. under N₂. Then the mixture was stirred at −75° C. for 2 h under N₂. The mixture was poured into NH₄Cl (aq, 50.0 mL) at 0° C. and extracted with EtOAc (2×30.0 mL). The combined organic layer was washed with saturated NaCl aqueous (100 mL), dried over Na₂SO₄ and concentrated under reduced pressure to get residue. The residue was purified by column chromatography (SiO₂, PE/EtOAc=100/0 to 98/2, TLC: PE/EtOAc=5/1, R_{f}=0.50) to give the title compound (570 mg, 2.14 mmol, 76.7% yield, 99.4% purity in LCMS at 220 nm) as a colorless oil. ¹H NMR: (400 MHz, DMSO-d₆) δ 7.57-7.51 (m, 3H), 7.45-7.43 (m, 2H), 7.39 (s, 1H), 2.94-2.83 (m, 1H), 1.10 (d, J=6.8 Hz, 6H). (ESI⁺) m/z: 266.7 (M+H)⁺, (C₁₂H₁₃N₂Br).

E. 3-(5-(2-Isopropyl-1-phenyl-1H-imidazol-4-yl)-1-oxoisoindolin-2-yl)piperidine-2,6-dione: To a solution of 4-bromo-2-isopropyl-1-phenyl-1H-imidazole (200 mg, 749 μmol, 1.00 eq) and 3-(1-oxo-5-(4,4,5,5-tetramethyl-1,3,2-dioxaborolan-2-yl)isoindolin-2-yl)piperidine-2,6-dione (693 mg, 1.87 mmol, 2.50 eq) in dioxane (5.00 mL) and H₂O (0.25 mL) was added K₃PO₄ (318 mg, 1.50 mmol, 2.00 eq) and Ru-Phos-Pd-G₃ (62.7 mg, 74.9 μmol, 0.10 eq) at 25° C. under N₂. Then the mixture was stirred at 100° C. for 3 h under N₂. The reaction mixture was filtered to get the filtrate, the filtrate was concentrated under vacuum to give a residue at 45° C. The residue was purified by column chromatography (SiO₂, DCM/MeOH=100/0 to 97.5/2.5, TLC: DCM/MeOH=15/1, R_{f}=0.40) to get a residue. The residue was purified by preparative-HPLC (using a Welch Xtimate C18 (150 mm×25 mm×5 μm) and gradient of 10-40% acetonitrile in water containing 0.05% TFA over 15 min at a flow rate of 25 mL/min) to give the title compound (169 mg, 395 μmol, 52.7% yield, 99.9% purity in HPLC at 220 nm) as a white solid. ¹H NMR: (400 MHz, DMSO-d₆) δ 11.0 (s, 1H), 8.25 (s, 1H), 8.10 (s, 1H), 8.01 (d, J=8.8 Hz, 1H), 7.84 (d, J=8.0 Hz, 1H), 7.68-7.61 (m, 5H), 5.16 (dd, J=4.8 Hz, J=13.2 Hz, 1H), 4.55-4.38 (m, 2H), 3.11-3.04 (m, 1H), 2.92-2.90 (m, 1H), 2.63-2.59 (m, 1H), 2.46-2.42 (m, 1H), 2.05-2.02 (m, 1H), 1.30 (d, J=6.8 Hz, 6H). (ESI⁺) m/z: 429.2 (M+H)⁺, (C₂₅H₂₄N₄O₃).

### Example 729

### Synthesis of 3-(5-(4-methyl-1-phenyl-1H-imidazol-2-yl)-1-oxoisoindolin-2-yl)piperidine-2,6-dione

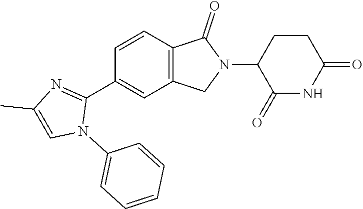 [SMILES: Cc1cn(-c2ccccc2)c(-c2ccc3c(c2)CN(C2CCC(=O)NC2=O)C3=O)n1]

A. 4-Methyl-1-phenyl-1H-imidazole: A solution of Pd₂(dba)₃ (348 mg, 381 μmol, 0.00750 eq) and Me₄tBuXPhos (439 mg, 914 μmol, 0.0180 eq) in dioxane (8.50 mL) and toluene (42.5 mL) was stirred at 120° C. for 10 mins under N₂. To a mixture of 4-methyl-1H-imidazole (5.02 g, 61.1 mmol, 1.20 eq) and K₃PO₄ (21.5 g, 101 mmol, 2.00 eq) was added bromobenzene (7.98 g, 50.8 mmol, 5.35 mL, 1.00 eq) at 25° C. under N₂. Then the solution 1 was added to the mixture and stirred at 120° C. for 5 h. The mixture was poured into H₂O (200 mL) and extracted with ethyl acetate (2×200 mL) to collect the organic layers, dried over anhydrous Na₂SO₄, filtered and the filtrate was concentrated under reduced pressure to get a residue. The residue was purified by column chromatography (SiO₂, DCM/MeOH═I/O to 100/1, TLC: DCM/MeOH=15/1, R_{f}=0.30) to give the title compound (5.00 g, 31.1 mmol, 61.2% yield, 98.5% purity in LCMS at 220 nm) as a yellow solid. ¹H NMR: (400 MHz, CDCl₃) δ 7.75 (s, 1H), 7.47-7.32 (m, 2H), 7.35-7.30 (m, 3H), 7.00 (s, 1H), 2.30 (s, 3H). (ESI⁺) m/z: 159.3 (M+H)⁺, (C₁₀H₁₀N₂).

B. 2-Bromo-4-methyl-1-phenyl-1H-imidazole: To a solution of 4-methyl-1-phenyl-imidazole (1.00 g, 6.23 mmol, 1.00 eq) in THF (10.0 mL) was added dropwise n-BuLi (2.50 M, 2.74 mL, 1.10 eq) at −78° C. under N₂. The reaction mixture was stirred 0.5 h, then 1,2-dibromo-1,1,2,2-tetrafluoro-ethane (1.70 g, 6.54 mmol, 1.05 eq) in THF (10.0 mL) was added dropwise at −78° C. under N₂. Then the reaction mixture was stirred at 25° C. for 3 h. The mixture was quenched with 10.0 mL of saturated NH₄Cl solution at 0° C. under N₂. Then the mixture extracted with ethyl acetate (3×20 mL) to collect the organic layers, dried over anhydrous Na₂SO₄, filtered and the filtrate was concentrated under reduced pressure to get a residue. The residue was purified by column chromatography (SiO₂, DCM/MeOH═I/O to 50/1, TLC: DCM/MeOH=15/1, R_{f}=0.30) to give the compound (880 mg, 3.63 mmol, 58.3% yield, 97.9% purity in LCMS at 220 nm) as a yellow solid. ¹H NMR: (400 MHz, CDCl₃) δ 7.50-7.42 (m, 3H), 7.37-7.33 (m, 2H), 6.88 (s, 1H), 2.26 (s, 3H). (ESI⁺) m/z: 236.9 (M+H)⁺, (C₁₀H₉N₂Br).

C. 3-(5-(4-Methyl-1-phenyl-1H-imidazol-2-yl)-1-oxoisoindolin-2-yl)piperidine-2,6-dione: To a solution of 2-bromo-4-methyl-1-phenyl-imidazole (200 mg, 825 μmol, 1.00 eq) and 3-[1-oxo-5-(4,4,5,5-tetramethyl-1,3,2-dioxaborolan-2-yl)isoindolin-2-yl]piperidine-2,6-dione (611 mg, 1.65 mmol, 2.00 eq) in dioxane (4.00 mL) and H₂O (0.200 mL) was added K₃PO₄ (350 mg, 1.65 mmol, 2.00 eq) and RuPhos-Pd-G₃ (69.0 mg, 82.5 μmol, 0.10 eq) at 25° C. The mixture was stirred at 100° C. for 2 h. The mixture was filtered and the filtrate was concentrated under vacuum at 45° C. to get a residue. The residue was purified by column chromatography (SiO₂, DCM/MeOH═I/O to 100/1, TLC: DCM/MeOH=15/1, R_{f}=0.30). The residue was purified by preparative-HPLC (using a Phenomenex luna C18 (150×25 mm×10 μm) and gradient of 3%-33% acetonitrile in water containing 0.05% FA over 15 min at a flow rate of 25 mL/min) to give the title compound (120 mg, 301 μmol, 36.4% yield, 100% purity in HPLC at 220 nm) as a white solid. ¹H NMR: (400 MHz, DMSO) δ 11.0 (s, 1H), 7.78-7.69 (m, 3H), 7.54-7.53 (m, 3H), 7.46-7.44 (m, 3H), 5.13-5.08 (m, 1H), 4.46-4.39 (m, 1H), 4.33-4.26 (m, 1H), 2.97-2.82 (m, 1H), 2.61-2.57 (m, 1H), 2.44-2.33 (m, 4H), 2.02-1.99 (m, 1H). (ESI⁺) m/z: 400.1 (M+H)⁺, (C₂₃H₂₀N₄O₃).

### Example 730

### Synthesis of 3-(5-(4-ethyl-1-phenyl-1H-imidazol-2-yl)-1-oxoisoindolin-2-yl)piperidine-2,6-dione

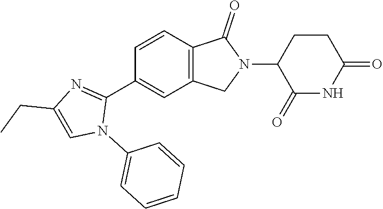 [SMILES: CCc1cn(-c2ccccc2)c(-c2ccc3c(c2)CN(C2CCC(=O)NC2=O)C3=O)n1]

A. 4-Ethyl-1-phenyl-1H-imidazole: A solution of Pd₂(dba)₃ (285 mg, 311 μmol, 0.0075 eq) and Me₄tBuXPhos (359 mg, 747 μmol, 0.018 eq) in toluene (35.5 mL) and dioxane (7.10 mL) was stirred at 120° C. for 10 mins under N₂. To a mixture of 4-ethyl-1H-imidazole (4.79 g, 49.83 mmol, 1.2 eq) and K₃PO₄ (17.6 g, 83.0 mmol, 2.00 eq) was added bromobenzene (6.52 g, 41.5 mmol, 4.37 mL, 1.00 eq) at 25° C. under N₂. Then the solution 1 was added to the mixture and stirred at 120° C. for 5 h. The mixture was poured into water (100 mL) and extracted with ethyl acetate (50.0 mL×2) to collect the organic layers. The combined organic layers were dried over anhydrous Na₂SO₄, filtered and the filtrate was concentrated under reduced pressure to get a residue. The crude product was purified by column chromatography (SiO₂, Dichloromethane/Methanol=100/1 to 30/1, TLC: Dichloromethane/Methanol=20/1, R_{f}=0.29) to give the title compound (3.00 g, 16.67 mmol, 40.1% yield, 95.7% purity in LCMS at 220 nm) as a yellow liquid. (ESI⁺) m/z: 172.9 (M+H)⁺ (C₁₁H₁₂N₂).

B. 2-Bromo-4-ethyl-1-phenyl-1H-imidazole: To a solution of 4-ethyl-1-phenyl-imidazole (1.00 g, 5.81 mmol, 1.00 eq) in THF (10.0 mL) was added dropwise n-BuLi (2.50 M, 2.55 mL, 1.10 eq) at −78° C. under N₂. The reaction mixture was stirred at −78° C. for 0.5 h, then 1,2-dibromo-1,1,2,2-tetrafluoro-ethane (1.58 g, 6.10 mmol, 1.05 eq) was added dropwise to the mixture at −78° C. Then the reaction mixture was stirred at 25° C. for 12 h. The reaction mixture was quenched with 50.0 mL of saturated NH₄Cl solution at 0° C. under N₂. Then the mixture was extracted with EtOAc (20.0 mL×3). The combined organic layers were washed with brine (100 mL), dried over Na₂SO₄, filtered and concentrated in vacuum to get a residue. The residue was purified by Preparative-HPLC (using a Phenomenex luna C18 (150 mm×25 mm×10 μm) and gradient of 7-37% acetonitrile in water containing 0.05% FA over 15 min at a flow rate of 25 mL/min) to give the title compound (100 mg, 238 μmol, Crude) as a yellow oil. ¹H NMR: (400 MHz, CDCl₃) δ 7.50-7.37 (m, 5H), 6.89 (s, 1H), 2.68 (q, J₁=7.6 Hz, J₂=15.2 Hz, 2H), 1.26 (d, J=7.6 Hz, 3H). (ESI⁺) m/z: 253.0 (M+H)⁺ (C₁₁H₁₁N₂Br).

C. 3-(5-(4-Ethyl-1-phenyl-1H-imidazol-2-yl)-1-oxoisoindolin-2-yl)piperidine-2,6-dione: To a solution of 2-bromo-4-ethyl-1-phenyl-imidazole (60.0 mg, 238 μmol, 1.00 eq) and 3-[1-oxo-5-(4,4,5,5-tetramethyl-1,3,2-dioxaborolan-2-yl)isoindolin-2-yl]piperidine-2,6-dione (176 mg, 477 μmol, 2.00 eq) in dioxane (2.00 mL) and H₂O (0.100 mL) was added K₃PO₄ (101 mg, 477 μmol, 2.00 eq) and RuPhos-Pd-G₃ (19.9 mg, 23.8 μmol, 0.10 eq) at 25° C. under N₂. The mixture was stirred for 2 h at 100° C. under N₂. The mixture was filtered and the filtrate was concentrated under reduced pressure to get a residue. The crude product was purified by column chromatography (SiO₂, Dichloromethane/Methanol=100/1 to 30/1, TLC:Dichloromethane/Methanol=15/1, R_{f}=0.29) to get a residue. The residue was purified by Preparative-HPLC (using a Phenomenex luna C18 (150 mm×25 mm×10 μm) and gradient of 3-33% acetonitrile in water containing 0.05% FA over 15 min at a flow rate of 25 mL/min) to give the title compound (34.66 mg, 83.3 μmol, 34.8% yield, 99.6% purity in HPLC at 220 nm) as a white solid. ¹H NMR: (400 MHz, DMSO-d₆) δ 10.9 (s, 1H) 7.60-7.45 (m, 5H), 7.30-7.27 (m, 4H), 5.11-5.06 (m, 1H), 4.41-4.23 (m, 2H), 2.88-2.63 (m, 1H), 2.59-2.55 (m, 3H), 2.39-2.32 (m, 1H), 2.00-1.99 (m, 1H), 1.26 (d, J=7.6 Hz, 3H). (ESI⁺) m/z: 415.1 (M+H)⁺, (C₂₄H₂₂N₄O₃).

### Example 731

### Synthesis of 3-(5-(4-isopropyl-1-phenyl-1H-imidazol-2-yl)-1-oxoisoindolin-2-yl)piperidine-2,6-dione

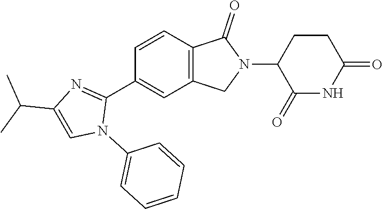 [SMILES: CC(C)c1cn(-c2ccccc2)c(-c2ccc3c(c2)CN(C2CCC(=O)NC2=O)C3=O)n1]

A. 4-Isopropyl-1-phenyl-1H-imidazole: A solution of Pd₂(dba)₃ (104 mg, 113 μmol, 0.00750 eq) and Me₄tBuXPhos (131 mg, 272 μmol, 0.018 eq) in toluene (13.0 mL) and dioxane (2.60 mL) was stirred at 120° C. for 3 min under N₂. Then the solution was added to the mixture of 4-isopropyl-1H-imidazole (2.00 g, 18.2 mmol, 1.20 eq), bromobenzene (2.38 g, 15.1 mmol, 1.59 mL, 1.00 eq) and K₃PO₄ (6.42 g, 30.3 mmol, 2.00 eq) at 25° C. The mixture was stirred at 120° C. for 5 h under N₂. The mixture was concentrated under vacuum to get a residue. The residue was purified by column chromatography (SiO₂, Dichloromethane/Methanol=100/1 to 15/1, TLC:Dichloromethane/Methanol=15/1, R_{f}=0.26) to give the title compound (2.40 g, 12.8 mmol, 84.7% yield, 99.4% purity in LCMS at 220 nm) as a light yellow oil. ¹H NMR: (400 MHz, CDCl₃) δ 7.51-27.48 (m, 2H), 7.44-7.42 (m, 3H), 6.70-6.99 (m, 2H), 1.80-1.74 (m, 1H), 1.13-21.09 (m, 3H), 0.91-0.87 (m, 3H). (ESI⁺) m/z: 187.2 (M+H)⁺, (C₁₂H₁₄N₂)

B. 2-Bromo-4-isopropyl-1-phenyl-1H-imidazole: To a solution of 4-isopropyl-1-phenyl-1H-imidazole (400 mg, 2.13 mmol, 1.00 eq) in THF (4.00 mL) was added n-BuLi (2.5 M, 939 μL, 1.10 eq) dropwise at −78° C. under N₂. The mixture was stirred at −78° C. for 0.5 h, then a solution of 1,2-dibromo-1,1,2,2-tetrafluoroethane (582 mg, 2.24 mmol, 1.05 eq) in THF (1.00 mL) was added dropwise at −78° C. Then the mixture was stirred at 25° C. for 4 h. The reaction mixture was quenched with 10.0 mL of saturated NH₄Cl solution at 0° C. under N₂. Then the mixture was extracted with ethyl acetate (3×20.0 mL). The combined organic layers were washed with brine (20.0 mL) dried over Na₂SO₄, filtered and concentrated in vacuum to get a residue. The residue was purified by preparative-TLC (SiO₂, Petroleum ether:Ethyl acetate=5:1, R_{f}=0.39) to give the title compound (220 mg, 824 μmol, 38.6% yield, 99.3% purity in LCMS at 220 nm) as a light yellow oil. ¹H NMR: (400 MHz, DMSO-d₆) δ 7.57-7.45 (m, 5H), 7.28-7.27 (m, 1H), 2.84-2.77 (m, 1H), 1.21 (s, 3H), 1.19 (s, 3H). (ESI⁺) m/z: 266.9 (M+H)⁺, (C₁₂H₁₃N₂Br).

C. 3-(5-(4-Isopropyl-1-phenyl-1H-imidazol-2-yl)-1-oxoisoindolin-2-yl)piperidine-2,6-dione: To a solution of 2-bromo-4-isopropyl-1-phenyl-1H-imidazole (200 mg, 749 μmol, 1.00 eq) and 3-(1-oxo-5-(4,4,5,5-tetramethyl-1,3,2-dioxaborolan-2-yl)isoindolin-2-yl)piperidine-2,6-dione (555 mg, 1.50 mmol, 2.00 eq) in dioxane (5.00 mL) and H₂O (0.25 mL) was added K₃PO₄ (318 mg, 1.50 mmol, 2.00 eq) and RuPhos Pd G₃ (62.6 mg, 74.9 μmol, 0.10 eq) at 25° C. under N₂. The mixture was stirred at 100° C. for 2.5 h. The mixture was concentrated under vacuum to get a residue at 50° C. The residue was purified by column chromatography (SiO₂, Dichloromethane/Methanol=100/0 to 100/3, TLC: Dichloromethane/Methanol=20/1, R_{f}=0.21). The solution was concentrated under vacuum to get a residue. The residue was purified by preparative-HPLC (using a Welch Xtimate C18 (150 mm×25 mm×5 μm) and gradient of 9-39% acetonitrile in water containing 0.05% TFA over 10 min at a flow rate of 30 mL/min) to give the title compound (203 mg, 468 μmol, 62.6% yield, 98.9% purity in HPLC at 220 nm) as a white solid. ¹H NMR: (400 MHz, DMSO-d₆) δ 11.0 (s, 1H), 7.78-7.76 (m, 2H), 7.72 (s, 1H), 7.54-7.52 (m, 3H), 7.48-7.43 (m, 3H), 5.11 (dd, J=5.2 Hz, J=13.2 Hz, 1H), 4.47-4.29 (m, 2H), 3.10-3.03 (m, 1H), 2.94-2.86 (m, 1H), 2.62-2.58 (m, 1H), 2.41-2.37 (m, 1H), 2.04-1.99 (m, 1H), 1.35 (d, J=6.8 Hz, 6H). (ESI⁺) m/z: 429.0 (M+H)⁺, (C₂₅H₂₄N₄O₃).

### Example 732

### Synthesis of 3-(5-(2-isobutyl-5-phenyloxazol-4-yl)-1-oxoisoindolin-2-yl)piperidine-2,6-dione

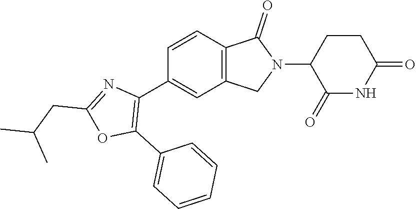 [SMILES: CC(C)Cc1nc(-c2ccc3c(c2)CN(C2CCC(=O)NC2=O)C3=O)c(-c2ccccc2)o1]

A. 2-Isobutyl-5-phenyloxazole: A mixture of acetophenone (5.00 g, 41.6 mmol, 4.86 mL, 1.00 eq), I₂ (21.1 g, 83.2 mmol, 2.00 eq) in DMSO (250 mL) was stirred at 110° C. for 1 h. Then to the mixture was added leucine (10.9 g, 83.2 mmol, 2.00 eq). The reaction mixture was stirred at 110° C. for 12 h. The reaction mixture was cooled down 25° C., then H₂O (250 mL) and brine (250 mL) were added to the reaction mixture. The mixture was extracted with ethyl acetate (3×200 mL), the organic layer was washed with aq. Na₂S₂O₃ (3×150 mL), dried over Na₂SO₄ and concentrated in vacuum to give residue. The residue was purified by column chromatography (SiO₂, Petroleum ether:Ethyl acetate=100:1 to 80:1; TLC, Petroleum ether:Ethyl acetate=10:1, R_{f}=0.45) to give the title compound (3.00 g, 14.9 mmol, 35.8% yield) as yellow oil. m/z: 202.1 (M+H)⁺, (C₁₃H₁₅NO).

B. 4-Bromo-2-isobutyl-5-phenyloxazole: To a solution of 2-isobutyl-5-phenyloxazole (1.00 g, 4.97 mmol, 1.00 eq) in DMF (10.0 mL) was added solution of NBS (972 mg, 5.47 mmol, 1.10 eq). The reaction mixture was stirred at 50° C. for 2 h. The reaction mixture was poured into H₂O (20.0 mL), extracted with ethyl acetate (3×50.0 mL). The combined organic layer was washed with H₂O (3×50.0 mL), dried over Na₂SO₄ and concentrated in vacuum to give residue. The residue was purified by column chromatography (SiO₂, Petroleum ether:Ethyl acetate=100:1 to 30:1; TLC, Petroleum ether:Ethyl acetate=10:1, R_{f}=0.60) to give the title compound (1.10 g, 3.93 mmol, 79.0% yield) as yellow oil. ¹H NMR: (400 MHz, CDCl₃) δ 7.92-7.90 (m, 2H), 7.47-7.45 (m, 2H), 7.43-7.36 (m, 1H), 2.70 (d, J=7.2 Hz, 2H), 2.25-2.18 (m, 1H), 1.03 (d, J=6.8 Hz, 6H). m/z: 280.3 (M+H)⁺, (C₁₃H₁₄BrNO).

C. Preparation of 3-(5-(2-isobutyl-5-phenyloxazol-4-yl)-1-oxoisoindolin-2-yl)piperidine-2,6-dione: To a solution of 4-bromo-2-isobutyl-5-phenyloxazole (0.50 g, 1.78 mmol, 1.00 eq) and 3-[1-oxo-5-(4,4,5,5-tetramethyl-1,3,2-dioxaborolan-2-yl)isoindolin-2-yl]piperidine-2,6-dione (1.65 g, 4.46 mmol, 2.50 eq), Ru-Phos-Pd-G₃ (149 mg, 178 μmol, 0.10 eq) and K₃PO₄ (757 mg, 3.57 mmol, 2.00 eq) in dioxane (10.0 mL) and H₂O (0.50 mL) under N₂. The reaction mixture was stirred at 100° C. for 2 h under N₂. The reaction mixture was concentrated in vacuum to give residue. The residue was purified by column chromatography (SiO₂, Dichloromethane:Methanol=100:1 to 80:1; TLC, Dichloromethane:Methanol=10:1, R_{f}=0.30) and preparative-HPLC (using a Welch Xtimate C18 (150 mm×25 mm×10 μm) and gradient of 37-67% acetonitrile in water containing 0.05% TFA over 15 min at a flow rate of 25.0 mL/min) to give the title compound (515 mg, 1.16 mmol, 64.7% yield, 99.5% purity in HPLC at 220 nm) as white solid. ¹H NMR: (400 MHz, DMSO-d₆) δ 10.9 (s, 1H), 7.82 (s, 1H), 7.75-7.69 (m, 2H), 7.56-7.54 (m, 2H), 7.48-7.45 (m, 3H), 5.14-5.10 (m, 1H), 4.49-4.32 (m, 2H), 2.94-2.91 (m, 1H), 2.74 (d, J=7.2 Hz, 2H), 2.63-2.57 (m, 1H), 2.41-2.38 (m, 1H), 2.25-2.15 (m, 1H), 2.03-2.01 (m, 1H), 1.01 (d, J=6.8 Hz, 6H). (ESI⁺) m/z: 444.3 (M+H)⁺, (C₂₆H₂₅N₃O₄).

### Example 733

### Synthesis of 3-(5-(2-isopropyl-5-phenyloxazol-4-yl)-1-oxoisoindolin-2-yl)piperidine-2,6-dione

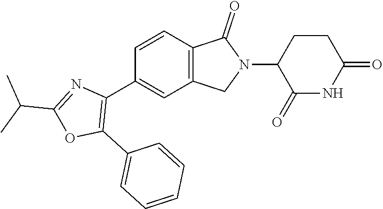 [SMILES: CC(C)c1nc(-c2ccc3c(c2)CN(C2CCC(=O)NC2=O)C3=O)c(-c2ccccc2)o1]

A. 2-Isopropyl-5-phenyloxazole: To a solution of 1-phenylethanone (2.00 g, 16.6 mmol, 1.95 mL, 1.00 eq) in DMSO (60.0 mL) was added 12 (8.45 g, 33.2 mmol, 6.71 mL, 2.00 eq) under N₂. The mixture was stirred at 110° C. for 1 h under N₂. Then 2-amino-3-methyl-butanoic acid (3.90 g, 33.29 mmol, 2.00 eq) was added to the mixture. The mixture was stirred at 110° C. for 0.5 h under N₂. After the reaction was completed, The mixture was poured into Na₂S₂O₃ (50.0 mL) and extracted with ethyl acetate (3×50.0 mL) to collect the organic layers, the combined organic layers were washed with brine (100.0 mL), dried over anhydrous Na₂SO₄, filtered and the filtrate was concentrated in vacuum to give a residue. The residue was purified by column chromatography (SiO₂, Petroleum ether:Ethyl acetate=100:1 to 5:1, R_{f}=0.50 (Petroleum ether:Ethyl acetate=5:1)) to give the title compound (1.60 g, 6.47 mmol, 38.8% yield, 75.7% purity in HPLC at 220 nm) as yellow oil. ¹H NMR: (400 MHz, CDCl₃) (7.68 (d, J=1.6 Hz, 2H), 7.59-7.50 (m, 1H), 7.48-7.43 (m, 2H), 7.38-7.33 (m, 1H), 3.26-3.14 (m, 1H), 1.65 (d, J=7.2 Hz, 6H). (ESI⁺) m/z: 188.1 (M+H)⁺, (C₁₂H₁₃NO).

B. 4-Bromo-2-isopropyl-5-phenyloxazole: To a solution of 2-isopropyl-5-phenyl-oxazole (1.50 g, 6.06 mmol, 1.00 eq) in ACN (15.0 mL) was added NBS (1.13 g, 6.37 mmol, 1.05 eq) at 0° C. The mixture was stirred at 25° C. for 3 h. After the reaction was completed, the reaction mixture was filtered and the filtrate was concentrated in vacuum to give a residue. The residue was purified column chromatography (SiO₂, Petroleum ether:Ethyl acetate=100:1 to 10:1, R_{f}=0.60 (Petroleum ether:Ethyl acetate=10:1)) to give the title compound (1.00 g, 3.62 mmol, 59.6% yield, 96.3% purity in HPLC at 220 nm) as yellow oil. ¹H NMR: (400 MHz, DMSO-d₆) δ 7.84 (d, J=8.0 Hz, 2H), 7.54-7.41 (m, 3H), 3.20-3.09 (m, 1H), 1.31 (d, J=6.8 Hz, 6H). (ESI⁺) m/z: 266.0 (M+H)⁺, (C₁₂H₁₂BrNO).

C. 3-(5-(2-Isopropyl-5-phenyloxazol-4-yl)-1-oxoisoindolin-2-yl)piperidine-2,6-dione: To a solution of 4-bromo-2-isopropyl-5-phenyl-oxazole (500 mg, 1.81 mmol, 1.00 eq) in dioxane (5.00 mL) and H₂O (0.25 mL) was added 3-[1-oxo-5-(4,4,5,5-tetramethyl-1,3,2-dioxaborolan-2-yl)isoindolin-2-yl]piperidine-2,6-dione (1.67 g, 4.52 mmol, 2.50 eq), Ru-Phos-Pd-G₃ (151 mg, 180 μmol, 0.10 eq) and K₃PO₄ (768 mg, 3.62 mmol, 2.00 eq) under N₂. The mixture was stirred at 100° C. for 2 h under N₂. After the reaction was completed, the reaction mixture was filtered and the filtrate was concentrated in vacuum to give a residue. The residue was purified by preparative-TLC (Dichloromethane:Methanol=10:1, R_{f}=0.40) and by preparative-HPLC (using a Phenomenex Luna C18 (150 mm×25 mm×5 μm) and gradient of 36-66% acetonitrile in water containing 0.5% FA over 10 min at a flow rate of 25.0 mL/min) to give the title compound (427 mg, 991 μmol, 54.7% yield, 99.5% purity in HPLC at 220 nm) as a white solid. ¹H NMR: (400 MHz, DMSO-d₆) δ 11.0 (s, 1H), 7.82 (s, 1H), 7.75-7.66 (m, 2H), 7.58-7.55 (m, 2H), 7.49-7.40 (m, 3H), 5.12 (dd, J=13.2, 5.2 Hz, 1H), 4.41 (dd, J=52.8, 17.6 Hz, 2H), 3.23-3.16 (m, 1H), 2.96-2.87 (m, 1H), 2.63-2.58 (m, 1H), 2.42-2.37 (m, 1H), 2.08-2.00 (m, 1H), 1.37 (d, J=6.8 Hz, 6H). (ESI⁺) m/z: 430.1 (M+H)⁺, (C₂₅H₂₃N₃O₄).

### Example 734

### Synthesis of 3-(5-(2-cyclopropyl-5-phenyloxazol-4-yl)-1-oxoisoindolin-2-yl)piperidine-2,6-dione

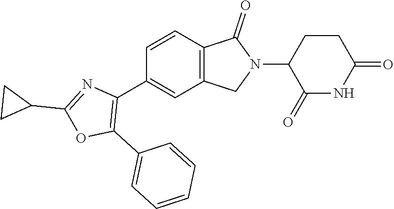 [SMILES: O=C1CCC(N2Cc3cc(-c4nc(C5CC5)oc4-c4ccccc4)ccc3C2=O)C(=O)N1]

A. 2-Cyclopropyl-5-phenyloxazole: To a solution of cyclopropanecarbonitrile (11.1 g, 166 mmol, 12.2 mL, 5.00 eq) in DCE (40.0 mL) was added PIDA (12.8 g, 39.9 mmol, 1.20 eq) and TfOH (19.9 g, 133 mmol, 11.7 mL, 4.00 eq) at 0° C. for 0.5 h. Then the mixture was added acetophenone (4 g, 33.2 mmol, 3.89 mL, 1.00 eq) at 0° C. Then the mixture was stirred at 80° C. for 3 h. The mixture was added TEA (2.00 mL). Then diluted with DCM. The combined organic layer was washed with saturated NaHCO₃ aqueous (100×3 mL), dried over Na₂SO₄ and concentrated under reduced pressure to get residue. The residue was purified by column chromatography (SiO₂, Petroleum ether:Ethyl acetate=100:1 to 0:1, TLC:Petroleum ether:Ethyl acetate=10:1, R_{f}=0.30) to give the title compound (900 mg, 4.51 mmol, 13.5% yield, 92.8% purity in LCMS at 220 nm) as light yellow oil. ¹H NMR: (400 MHz, DMSO-d₆) δ 7.67-7.63 (m, 2H), 7.48-7.38 (m, 3H), 7.34-7.32 (m, 1H), 2.19-2.12 (m, 1H), 1.09-1.05 (m, 2H), 1.01-0.99 (m, 2H) (ESI⁺) m/z: 185.1 (M+H)⁺, (C₁₂H₁₁NO).

B. 4-Bromo-2-cyclopropyl-5-phenyloxazole: To a solution of 2-cyclopropyl-5-phenyloxazole (500 mg, 2.70 mmol, 1.00 eq) in ACN (8.00 mL) was added NBS (528 mg, 2.97 mmol, 1.10 eq) at 0° C. The mixture was stirred at 25° C. for 2 h. The reaction mixture was poured into H₂O (50.0 mL) and extracted with Ethyl acetate (3×50.0 mL). The combined organic layer was washed with brine (3×50.0 mL), dried over Na₂SO₄, filtered and concentrated. The residue was purified by TLC (Petroleum ether:Ethyl acetate=10:1, R_{f}=0.50) to give the title compound (400 mg, 1.50 mmol, 55.6% yield, 99.2% purity in LCMS at 220 nm) as light yellow oil. (ESI⁺) m/z: 264.1 (M+H)⁺, (C₁₂H₁₀BrNO).

C. 3-(5-(2-Cyclopropyl-5-phenyloxazol-4-yl)-1-oxoisoindolin-2-yl)piperidine-2,6-dione: To a solution of 4-bromo-2-cyclopropyl-5-phenyloxazole (200 mg, 757 μmol, 1.00 eq), 3-(1-oxo-5-(4,4,5,5-tetramethyl-1,3,2-dioxaborolan-2-yl)isoindolin-2-yl)piperidine-2,6-dione (841 mg, 2.27 mmol, 3.00 eq) and K₃PO₄ (482 mg, 2.27 mmol, 3.00 eq) in dioxane (5.00 mL) and H₂O (0.25 mL) was added Ru-Phos-Pd-G₃ (63.3 mg, 75.2 μmol, 0.10 eq) under N₂. The mixture was stirred at 100° C. for 3 h under N₂. The reaction mixture was concentrated under reduced pressure to get a residue at 45° C. The residue was purified by preparative-HPLC (using a Phenomenex luna C18 (150×25 mm×10 μm) and gradient of 28.0%-58.0% acetonitrile in water containing 0.05% FA over 10 min at a flow rate of 25 mL/min to give the title compound (105 mg, 247 μmol, 32.6% yield, 100% purity in HPLC at 220 nm) as white solid. ¹H NMR: (400 MHz, DMSO-d₆) δ 10.99-10.96 (m, 1H), 7.79-7.72 (m, 1H), 7.66-7.64 (m, 1H), 7.55-7.53 (m, 1H), 7.50-7.54 (m, 2H), 7.44-7.42 (m, 3H), 5.14-5.08 (m, 1H), 4.49-4.31 (m, 2H), 2.94-2.90 (m, 1H), 2.62-2.57 (m, 1H), 2.41-2.38 (m, 1H), 2.23-2.20 (m, 1H), 2.03-2.01 (m, 1H), 1.12-1.03 (m, 4H). (ESI⁺) m/z: 428.1 (M+H)⁺, (C₂₅H₂IN₃O₄).

### Example 735

### Synthesis of 3-(5-(2-(difluoromethyl)-5-phenyloxazol-4-yl)-1-oxoisoindolin-2-yl) piperidine-2, 6-dione

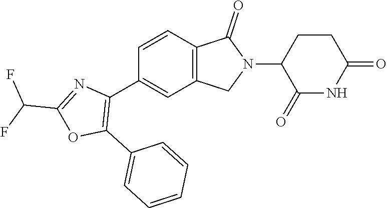 [SMILES: O=C1CCC(N2Cc3cc(-c4nc(C(F)F)oc4-c4ccccc4)ccc3C2=O)C(=O)N1]

A. 2,2-Difluoro-N-(2-oxo-2-phenylethyl) acetamide: To a solution of 2-amino-1-phenylethan-1-one (5.00 g, 29.1 mmol, 1.00 eq, HCl) in THF (100 mL) was added TEA (5.90 g, 58.3 mmol, 8.11 mL, 2.00 eq) and 2,2-difluoroacetic anhydride (8.62 g, 49.5 mmol, 1.70 eq). Then the mixture was stirred at 25° C. for 2 h. The reaction mixture was filtered and the filtrate was concentrated under vacuum to give the title compound (3.50 g, 16.4 mmol, 56.3% yield) as yellow oil. m/z: 214.0 (M+H)⁺, (C₁₀H₉F₂NO₂).

B. 2-(Difluoromethyl)-5-phenyloxazole: To a solution of 2, 2-difluoro-N-(2-oxo-2-phenylethyl) acetamide (3.50 g, 16.4 mmol, 1.00 eq) in H₂SO₄ (8.05 g, 82.1 mmol, 4.38 mL, 5.00 eq). Then the mixture was stirred at 80° C. for 2 h. The mixture was poured into water (20 mL) at 0° C. Then the solution was neutralized with 28% NH₃·H₂O and extracted with ethyl acetate (3×50 mL). Combined organic layer was washed with brine (3×30.0 mL), dried over by Na₂SO₄, filtered and the filtrate was concentrated in vacuum to get residue. The residue was purified by column chromatography (SiO₂, Petroleum ether:Ethyl acetate=100:1 to 1:1, TLC:Petroleum ether:Ethyl acetate=5:1, R_{f}=0.30) to give the title compound (1.10 g, 5.38 mmol, 16.3% yield, 95.4% purity in LCMS at 220 nm) as yellow solid. m/z: 196.0 (M+H)⁺, (C₁₀H₇F₂NO).

C. 4-Bromo-2-(difluoromethyl)-5-phenyloxazole: To a solution of 2-(difluoromethyl)-5-phenyloxazole (1.10 g, 5.64 mmol, 1.00 eq) in ACN (10.0 mL) was added NBS (1.10 g, 6.20 mmol, 1.10 eq). Then the mixture was stirred at 25° C. for 2 h. The reaction was concentrated under reduce pressure to get residue. The residue was purified by column chromatography (SiO₂, Petroleum ether:Ethyl acetate=100:1 to 1:1, TLC: Petroleum ether:Ethyl acetate=5:1, R_{f}=0.50) to give the title compound (1.00 g, 3.48 mmol, 61.7% yield, 95.4% purity in LCMS at 220 nm). m/z: 273.8 (M+H)⁺, (C₁₀H₆BrF₂NO).

D. 3-(5-(2-(Difluoromethyl)-5-phenyloxazol-4-yl)-1-oxoisoindolin-2-yl)piperidine-2,6-dione: To a solution of 4-bromo-2-(difluoromethyl)-5-phenyloxazole (200 mg, 729 μmol, 1.00 eq), 3-(1-oxo-5-(4,4,5,5-tetramethyl-1,3,2-dioxaborolan-2-yl)isoindolin-2-yl)piperidine-2,6-dione (810 mg, 2.19 mmol, 3.00 eq) and K₃PO₄ (464 mg, 2.19 mmol, 3.00 eq) in dioxane (5.00 mL) and H₂O (0.25 mL) was added Ru-Phos-Pd-G₃ (61.0 mg, 72.9 μmol, 0.10 eq) under N₂, The mixture was stirred at 100° C. for 2 h under N₂. The reaction mixture was concentrated under reduced pressure to get a residue at 45° C. The residue was purified by preparative-HPLC (using a Welch Xtimate C18 (150 mm×25 mm×10 μm) and gradient of 29-59% acetonitrile in water containing 0.05% TFA over 15 min at a flow rate of 25.0 mL/min) to give the title compound (175 mg, 401 μmol, 54.9% yield, 99.9% purity in HPLC at 220 nm) as white solid. ¹H NMR: (400 MHz, DMSO-d₆) δ 11.0 (s, 1H), 7.86 (s, 1H), 7.77-7.71 (m, 1H), 7.70-7.67 (m, 1H), 7.63-7.61 (m, 2H), 7.53-7.51 (m, 3H), 7.37-7.25 (m, 1H), 5.16-5.11 (m, 1H), 4.52-4.34 (m, 2H), 2.96-2.93 (m, 1H), 2.63-2.59 (m, 1H), 2.42-2.39 (m, 1H), 2.04-2.01 (m, 1H). (ESI⁺) m/z: 438.1 (M+H)⁺, (C₂₃H₁₇F₂N₃O₄).

### Example 736

HiBit Assays

Procedure A

CSNK1A1-HiBit KI HEK293(LgBit) Cells (Promega Catalog #CS3023103; Lot #0000449125) and HiBit-GSPT1 KI HEK293(LgBit) Cells (Promega Catalog #CS302381; Lot #0000381455) were maintained in DMEM (Thermo Fisher Scientific, Catalog #11965126) containing 10% Fetal Bovine Serum (Thermo Fisher Scientific, Catalog #16140071) 1% Penicillin-Streptomycin (Thermo Fisher Scientific, Catalog #25200056) and 200 μg/ml Hygromycin (Thermo Fisher Scientific, Catalog #10687010). Cells were maintained at 37° C. in a 5% CO₂ environment.

Compounds were spotted onto Cornin 384-Well, Cell Culture-Treated, Flat-Bottom, Low Flange Microplate (Fisher Scientific, Catalog #07-201-013) using a Tecan D300e digital dispenser. Ten-point dose response curves spanning from 10 μM to 0.005 μM with a 0.1% DMSO tolerance were used.

Cells were dissociated from tissue 182 cm² Tissue Culture Flask (CellTreat Catalog #229351) by washing the cells with PBS (Fisher Scientific Catalog #BP3994) followed by dissociation with Trypsin-EDTA (0.25%) (Thermo Scientific Catalog #25200056). Trysin was neutralized and cells were resuspended with DMEM+10% FBS. Cells were centrifuged for 5 minutes at 1200 RPMs. Cell culture media was aspirated off and cells were resuspended with DMEM+10% FBS. Cell density was determined using a Denovix CellDrop BF cell counter. Cell density was adjusted to 1×106 cells/ml. 40 μl of cells was added to compound treated tissue culture plates and incubated for six hours at 37° C. After six hours, cell culture plates were removed from tissue culture incubator and cooled to room temperature. 40 μl of Nano-GloHiBiT Lytic Detection Reagent (Promega, Catalog #N3040) was added to each well. Plates were placed on an orbital shaker (600 rpm) for 20 minutes. Luminescence was measured using an Envsion plate reader (Perkin Elmer). DC₅₀ curves and Yₘᵢₙ were generated using a 4-parameter curve fit with GraphPad Prism software (Prism Corporation).

Procedure B

Molt 4_hGSPT1_HiBIT N-terminus tagged cells were generated using CRISPR technology. Cells were maintained in RPMI media (Thermo Fisher Scientific, Catalog #11875-093) containing 10% Fetal Bovine Serum (Thermo Fisher Scientific, Catalog #16140071) 1% Penicillin-Streptomycin (Thermo Fisher Scientific, Catalog #25200056). Cells were maintained at 37° C. in a 5% CO₂ environment.

Compounds were spotted onto Cornin 384-Well, Cell Culture-Treated, Flat-Bottom, Low Flange Microplate (Fisher Scientific, Catalog #07-201-013) using a Tecan D300e digital dispenser. Ten-point dose response curves spanning from 10 μM to 0.005 μM with a 0.1% DMSO tolerance were used.

Cells were centrifuged for 5 minutes at 1200 RPMs. Cell culture media was aspirated off and cells were resuspended with RPMI+10% FBS. Cell density was determined using a Denovix CellDrop BF cell counter. Cell density was adjusted to 1×10⁶ cells/ml. 40 μl of cells was added to compound treated tissue culture plates and incubated for six hours at 37° C. After six hours, cell culture plates were removed from tissue culture incubator and cooled to room temperature. 40 μl of Nano-GloHiBiT Lytic Detection Reagent (Promega, Catalog #N3040) was added to each well. Plates were placed on an orbital shaker (600 rpm) for 20 minutes. Luminescence was measured using an Envsion plate reader (Perkin Elmer). IC₅₀ curves and Yₘᵢₙ were generated using a 4-parameter curve fit with GraphPad Prism software (Prism Corporation).

Results

Results from the HiBiT assay (DC₅₀ (nM) and Yₘᵢₙ (% remaining)) are shown below. All CK1α data was generated according to Procedure A. GSPT1 data for Examples 1-600, 603-620 and 703-705 was generated according to Procedure A. GSPT1 data for Examples 601, 621-702 and 706-735 was generated according to Procedure B.

| | CK1α | CK1α | GSPT1 | GSPT1 |
|---|---|---|---|---|
| Ex. | DC₅₀ | Yₘᵢₙ | DC₅₀ | Yₘᵢₙ |
| 1 | A | D | B | F |
| 2 | A | C | A | E |
| 3 | A | E | B | F |
| 4 | B | E | B | F |
| 5 | A | D | B | F |
| 6 | B | E | B | F |
| 7 | A | C | A | E |
| 8 | A | E | B | F |
| 9 | B | E | B | E |
| 10 | A | C | B | E |
| 11 | A | C | A | D |
| 12 | A | C | B | E |
| 13 | A | C | A | E |
| 14 | A | C | B | E |
| 15 | A | C | A | E |
| 16 | B | E | B | F |
| 17 | A | C | B | E |
| 18 | B | E | B | E |
| 19 | A | C | B | E |
| 20 | B | E | B | E |
| 21 | B | E | B | C |
| 22 | A | C | A | E |
| 23 | A | D | B | E |
| 24 | A | C | B | E |
| 25 | A | C | B | F |
| 26 | A | C | A | E |
| 27 | A | D | B | F |
| 28 | B | E | B | F |
| 29 | B | F | B | F |
| 30 | B | E | B | F |
| 31 | B | E | B | F |
| 32 | A | D | A | E |
| 33 | A | E | B | E |
| 34 | A | D | B | F |
| 35 | B | F | B | F |
| 36 | A | D | B | F |
| 37 | A | D | B | F |
| 38 | B | E | B | F |
| 39 | A | E | B | F |
| 40 | A | D | A | D |
| 41 | A | D | B | F |
| 42 | A | E | B | F |
| 43 | A | D | B | F |
| 44 | A | C | B | F |
| 45 | A | C | B | F |
| 46 | A | C | B | F |
| 47 | B | E | B | F |
| 48 | A | C | A | D |
| 49 | A | C | A | F |
| 50 | A | C | B | F |
| 51 | A | C | B | F |
| 52 | A | C | A | F |
| 53 | A | C | B | E |
| 54 | A | C | B | F |
| 55 | A | E | B | F |
| 56 | B | D | B | F |
| 57 | B | E | B | F |
| 58 | B | E | B | F |
| 59 | A | E | B | F |
| 60 | A | E | B | F |
| 61 | A | E | B | F |
| 62 | A | D | B | F |
| 63 | A | E | B | F |
| 64 | A | C | B | F |
| 65 | A | D | B | F |
| 66 | B | E | B | F |
| 67 | A | E | B | F |
| 68 | A | D | B | F |
| 69 | A | E | B | F |
| 70 | A | C | B | F |
| 71 | A | F | B | F |
| 72 | A | D | B | F |
| 73 | A | E | B | F |
| 74 | A | E | B | F |
| 75 | B | E | B | F |
| 76 | A | D | B | F |
| 77 | A | E | B | F |
| 78 | A | E | B | F |
| 79 | A | E | B | F |
| 80 | A | E | B | F |
| 81 | B | E | B | F |
| 82 | A | E | B | F |
| 83 | A | E | B | F |
| 84 | A | E | B | F |
| 85 | A | E | B | F |
| 86 | A | E | B | F |
| 87 | A | E | B | F |
| 88 | A | E | B | F |
| 89 | A | E | B | F |
| 90 | A | E | B | F |
| 91 | A | E | B | E |
| 92 | B | E | B | F |
| 93 | B | E | B | F |
| 94 | A | D | B | F |
| 95 | A | D | B | F |
| 96 | B | E | B | F |
| 97 | A | D | B | F |
| 98 | B | E | B | F |
| 99 | A | E | B | F |
| 100 | B | E | B | F |
| 101 | A | E | B | F |
| 102 | B | E | B | F |
| 103 | A | D | B | F |
| 104 | A | C | B | F |
| 105 | A | E | B | F |
| 106 | A | E | B | F |
| 107 | A | E | B | F |
| 108 | A | E | B | F |
| 109 | A | E | B | F |
| 110 | A | E | B | F |
| 111 | B | F | B | F |
| 112 | A | D | B | F |
| 113 | A | D | B | F |
| 114 | A | E | B | F |
| 115 | A | E | B | F |
| 116 | B | E | B | F |
| 117 | A | C | B | F |
| 118 | B | E | B | F |
| 119 | B | E | B | F |
| 120 | B | E | B | F |
| 121 | B | C | B | F |
| 122 | B | E | B | F |
| 123 | A | E | B | F |
| 124 | A | C | B | F |
| 125 | A | E | B | F |
| 126 | A | D | B | F |
| 127 | A | E | B | F |
| 128 | A | D | B | F |
| 129 | A | E | B | F |
| 130 | A | E | B | F |
| 131 | A | E | B | F |
| 132 | A | E | B | F |
| 133 | B | E | B | F |
| 134 | A | E | B | F |
| 135 | A | E | B | F |
| 136 | A | D | B | F |
| 137 | A | E | A | F |
| 138 | B | E | A | F |
| 139 | B | E | A | F |
| 140 | B | E | A | E |
| 141 | A | E | B | F |
| 142 | A | D | B | F |
| 143 | A | D | B | F |
| 144 | A | E | B | F |
| 145 | A | C | A | F |
| 146 | A | D | B | E |
| 147 | A | C | B | F |
| 148 | A | E | A | F |
| 149 | A | D | A | F |
| 150 | A | D | B | F |
| 151 | A | E | B | F |
| 152 | B | E | B | F |
| 153 | A | D | B | F |
| 154 | B | E | B | F |
| 155 | A | E | B | F |
| 156 | B | D | B | F |
| 157 | B | E | B | F |
| 158 | B | E | B | F |
| 159 | A | D | B | F |
| 160 | B | E | B | C |
| 161 | A | D | B | F |
| 162 | A | C | A | E |
| 163 | A | C | B | E |
| 164 | A | C | A | E |
| 165 | A | D | B | F |
| 166 | A | E | B | E |
| 167 | A | E | A | F |
| 168 | A | C | A | D |
| 169 | A | E | B | F |
| 170 | A | D | B | F |
| 171 | A | D | A | F |
| 172 | A | E | B | F |
| 173 | A | E | B | F |
| 174 | A | D | A | E |
| 175 | A | D | B | F |
| 176 | A | D | A | E |
| 177 | A | D | A | E |
| 178 | A | D | A | F |
| 179 | A | D | A | E |
| 180 | B | E | B | F |
| 181 | A | C | B | E |
| 182 | A | E | B | F |
| 183 | A | E | B | F |
| 184 | A | D | B | C |
| 185 | A | D | B | F |
| 186 | B | E | B | F |
| 187 | A | D | B | F |
| 188 | A | C | A | E |
| 189 | A | C | A | E |
| 190 | A | E | A | E |
| 191 | A | E | A | E |
| 192 | A | E | A | E |
| 193 | A | D | A | F |
| 194 | A | C | A | E |
| 195 | A | D | A | E |
| 196 | A | E | A | F |
| 197 | A | E | A | E |
| 198 | A | C | A | E |
| 199 | A | D | A | F |
| 200 | A | D | A | E |
| 201 | A | E | B | F |
| 202 | A | D | B | F |
| 203 | A | D | B | E |
| 204 | A | E | B | F |
| 205 | A | E | B | F |
| 206 | B | E | B | F |
| 207 | A | D | B | F |
| 208 | A | D | B | F |
| 209 | A | C | B | F |
| 210 | A | D | B | F |
| 211 | A | C | B | F |
| 212 | A | D | B | F |
| 213 | A | E | B | F |
| 214 | A | E | B | F |
| 215 | A | D | B | F |
| 216 | A | E | B | F |
| 217 | A | D | B | F |
| 218 | B | D | B | F |
| 219 | A | C | B | F |
| 220 | A | E | B | F |
| 221 | B | E | B | F |
| 222 | A | C | B | F |
| 223 | B | E | B | F |
| 224 | A | C | B | F |
| 225 | B | E | B | F |
| 226 | A | C | A | E |
| 227 | A | D | A | E |
| 228 | A | C | B | D |
| 229 | A | C | A | E |
| 230 | A | C | A | E |
| 231 | A | D | B | F |
| 232 | A | D | B | F |
| 233 | A | D | B | F |
| 234 | A | D | B | F |
| 235 | A | D | B | F |
| 236 | A | C | B | E |
| 237 | A | E | B | F |
| 238 | A | D | B | F |
| 239 | A | C | A | F |
| 240 | A | D | A | E |
| 241 | A | D | B | F |
| 242 | A | D | A | F |
| 243 | B | E | B | F |
| 244 | A | D | B | F |
| 245 | A | E | B | F |
| 246 | A | E | B | F |
| 247 | A | E | B | — |
| 248 | B | D | B | E |
| 249 | A | D | B | F |
| 250 | A | C | A | E |
| 251 | A | D | B | F |
| 252 | A | D | B | E |
| 253 | A | C | A | D |
| 254 | A | E | A | E |
| 255 | A | E | A | F |
| 256 | A | C | A | E |
| 257 | A | D | B | F |
| 258 | A | D | B | E |
| 259 | A | D | A | E |
| 260 | A | D | A | F |
| 261 | A | E | B | F |
| 262 | A | D | B | F |
| 263 | B | E | A | F |
| 264 | B | E | A | E |
| 265 | A | D | B | F |
| 266 | B | E | A | F |
| 267 | B | C | B | F |
| 268 | B | D | B | F |
| 269 | A | C | A | F |
| 270 | A | D | B | F |
| 271 | B | E | A | F |
| 272 | A | E | A | F |
| 273 | A | C | A | E |
| 274 | A | E | B | F |
| 275 | A | D | B | F |
| 276 | A | C | A | F |
| 277 | A | E | A | F |
| 278 | A | E | B | F |
| 279 | A | D | B | F |
| 280 | A | E | B | F |
| 281 | A | E | A | F |
| 282 | A | E | B | F |
| 283 | A | E | B | F |
| 284 | A | E | B | F |
| 285 | A | E | A | F |
| 286 | A | D | A | E |
| 287 | A | D | A | F |
| 288 | A | C | A | E |
| 289 | B | C | B | F |
| 290 | B | D | B | F |
| 291 | A | E | B | F |
| 292 | A | C | A | E |
| 293 | B | E | A | F |
| 294 | A | D | A | E |
| 295 | B | D | B | F |
| 296 | A | E | B | F |
| 297 | A | E | B | F |
| 298 | A | E | B | F |
| 299 | A | C | B | F |
| 300 | B | D | B | F |
| 301 | A | D | B | F |
| 302 | B | E | B | F |
| 303 | A | E | B | F |
| 304 | A | D | A | F |
| 305 | A | C | A | F |
| 306 | B | E | B | E |
| 307 | B | D | A | F |
| 308 | A | D | B | F |
| 309 | A | D | A | F |
| 310 | A | D | B | F |
| 311 | A | D | A | E |
| 312 | B | D | B | F |
| 313 | B | D | B | F |
| 314 | B | E | B | F |
| 315 | A | D | B | F |
| 316 | A | D | A | F |
| 317 | A | D | B | F |
| 318 | A | E | A | F |
| 319 | A | C | B | E |
| 320 | A | D | B | F |
| 321 | A | D | B | F |
| 322 | A | C | A | F |
| 323 | B | E | B | F |
| 324 | B | E | B | F |
| 325 | B | E | B | F |
| 326 | A | C | A | F |
| 327 | A | D | A | F |
| 328 | B | D | B | F |
| 329 | A | C | B | E |
| 330 | A | E | B | F |
| 331 | A | D | B | F |
| 332 | A | D | B | F |
| 333 | A | E | B | E |
| 334 | B | E | A | F |
| 335 | A | C | A | F |
| 336 | B | D | B | F |
| 337 | B | D | B | F |
| 338 | A | C | B | F |
| 339 | A | C | A | F |
| 340 | A | E | A | F |
| 341 | A | E | B | F |
| 342 | B | F | B | F |
| 343 | A | C | B | F |
| 344 | A | E | B | F |
| 345 | A | E | B | F |
| 346 | A | D | B | F |
| 347 | A | E | B | F |
| 348 | A | E | B | F |
| 349 | A | C | A | F |
| 350 | B | D | B | F |
| 351 | B | D | B | F |
| 352 | A | C | B | F |
| 353 | A | C | A | F |
| 354 | A | E | A | F |
| 355 | A | E | B | F |
| 356 | B | F | B | F |
| 357 | A | C | B | F |
| 358 | A | E | B | F |
| 359 | A | E | B | F |
| 360 | A | D | B | F |
| 361 | A | E | B | F |
| 362 | A | E | B | F |
| 363 | A | C | A | F |
| 364 | B | D | B | F |
| 365 | B | D | B | F |
| 366 | A | C | B | F |
| 367 | A | C | A | F |
| 368 | A | E | A | F |
| 369 | B | F | B | F |
| 370 | B | E | B | |
| 371 | B | D | B | F |
| 372 | B | E | B | F |
| 373 | B | E | B | F |
| 374 | B | F | B | F |
| 375 | A | D | B | F |
| 376 | A | D | B | F |
| 377 | A | E | B | F |
| 378 | A | E | B | F |
| 379 | A | C | B | F |
| 380 | A | E | B | F |
| 381 | A | D | B | F |
| 382 | B | D | B | F |
| 383 | A | E | B | F |
| 384 | B | E | A | F |
| 385 | A | E | B | F |
| 386 | A | E | B | F |
| 387 | A | D | B | F |
| 388 | B | E | B | F |
| 389 | A | E | B | F |
| 390 | A | F | B | F |
| 391 | B | C | B | F |
| 392 | A | E | B | F |
| 393 | A | D | B | F |
| 394 | B | E | B | F |
| 395 | A | F | A | E |
| 396 | A | E | B | F |
| 397 | B | D | B | F |
| 398 | A | D | A | F |
| 399 | A | D | B | F |
| 400 | B | D | B | F |
| 401 | A | C | B | E |
| 402 | A | E | B | F |
| 403 | A | D | B | F |
| 404 | B | E | B | F |
| 405 | A | F | A | F |
| 406 | B | D | A | E |
| 407 | A | C | B | E |
| 408 | A | E | B | |
| 409 | B | E | B | F |
| 410 | A | E | B | F |
| 411 | A | E | B | F |
| 412 | B | E | B | F |
| 413 | B | E | B | F |
| 414 | B | E | B | F |
| 415 | A | E | B | F |
| 416 | A | E | B | F |
| 417 | A | D | B | F |
| 418 | A | C | B | E |
| 419 | A | C | B | F |
| 420 | A | C | B | F |
| 421 | A | C | B | F |
| 422 | A | D | A | F |
| 423 | A | D | B | F |
| 424 | A | D | B | F |
| 425 | A | D | B | |
| 426 | A | D | B | F |
| 427 | A | C | B | F |
| 428 | A | C | B | F |
| 429 | A | D | A | F |
| 430 | A | D | B | F |
| 431 | A | C | B | F |
| 432 | A | C | B | F |
| 433 | A | C | B | F |
| 434 | A | C | B | F |
| 435 | A | E | A | F |
| 436 | A | D | B | F |
| 437 | A | C | A | F |
| 438 | B | E | B | F |
| 439 | A | C | A | E |
| 440 | A | C | A | E |
| 441 | A | C | A | E |
| 442 | A | E | B | F |
| 443 | A | C | A | E |
| 444 | A | C | A | E |
| 445 | A | E | B | F |
| 446 | A | D | A | F |
| 447 | B | D | B | F |
| 448 | B | D | B | F |
| 449 | B | E | B | F |
| 450 | B | F | B | F |
| 451 | B | E | B | F |
| 452 | A | E | B | F |
| 453 | A | E | B | F |
| 454 | B | F | B | F |
| 455 | B | E | B | F |
| 456 | B | E | B | F |
| 457 | B | E | B | F |
| 458 | A | F | A | F |
| 459 | A | E | B | F |
| 460 | B | D | B | F |
| 461 | B | C | B | F |
| 462 | B | E | B | F |
| 463 | B | D | B | F |
| 464 | B | F | B | F |
| 465 | B | E | B | F |
| 466 | A | F | B | F |
| 467 | B | F | B | |
| 468 | B | E | B | F |
| 469 | B | D | B | F |
| 470 | B | E | B | F |
| 471 | A | C | B | F |
| 472 | A | E | B | F |
| 473 | A | E | B | F |
| 474 | A | D | B | F |
| 475 | B | E | A | F |
| 476 | B | D | B | F |
| 477 | A | C | B | F |
| 478 | A | D | B | F |
| 479 | A | E | B | F |
| 480 | B | E | B | F |
| 481 | B | E | A | F |
| 482 | A | D | A | E |
| 483 | A | D | A | F |
| 4584 | A | C | A | F |
| 485 | B | E | B | F |
| 486 | A | D | A | F |
| 487 | A | E | B | F |
| 488 | A | D | | |
| 489 | A | C | A | E |
| 490 | B | E | B | F |
| 491 | A | D | B | E |
| 492 | B | F | B | F |
| 493 | A | D | B | F |
| 494 | B | E | B | F |
| 495 | A | C | | |
| 496 | A | C | A | E |
| 497 | B | D | B | F |
| 498 | A | D | A | F |
| 499 | A | E | B | F |
| 500 | A | C | B | F |
| 501 | A | C | B | F |
| 502 | A | C | A | E |
| 503 | B | E | B | F |
| 504 | B | D | B | F |
| 505 | A | D | A | F |
| 506 | A | C | A | E |
| 507 | A | E | B | F |
| 508 | A | D | B | F |
| 509 | A | D | A | E |
| 510 | A | C | B | E |
| 511 | A | D | B | F |
| 512 | A | C | A | F |
| 513 | A | C | B | F |
| 514 | A | C | B | E |
| 515 | A | C | A | E |
| 516 | A | D | A | F |
| 517 | B | E | A | F |
| 518 | A | C | A | F |
| 519 | A | C | B | F |
| 520 | B | E | B | F |
| 521 | A | C | A | E |
| 522 | B | D | B | E |
| 523 | B | D | B | E |
| 524 | B | E | B | F |
| 525 | A | C | A | C |
| 526 | A | E | A | F |
| 527 | A | D | A | E |
| 528 | B | D | B | E |
| 529 | A | D | A | F |
| 530 | A | E | A | F |
| 531 | B | C | B | E |
| 532 | A | D | B | F |
| 533 | B | E | B | F |
| 534 | A | D | B | F |
| 535 | B | D | B | F |
| 536 | B | F | B | F |
| 537 | A | D | B | F |
| 538 | A | D | B | |
| 539 | A | E | B | F |
| 540 | A | C | B | F |
| 541 | A | D | A | F |
| 542 | A | C | A | F |
| 543 | A | C | B | F |
| 544 | B | E | B | F |
| 545 | A | D | B | F |
| 546 | A | D | B | F |
| 547 | A | D | B | F |
| 548 | A | D | B | F |
| 549 | A | C | B | F |
| 550 | A | C | B | F |
| 551 | A | E | B | F |
| 552 | B | E | B | F |
| 553 | A | C | B | F |
| 554 | A | D | B | F |
| 555 | A | C | B | F |
| 556 | A | C | B | F |
| 557 | A | C | B | F |
| 558 | A | C | B | F |
| 559 | A | C | A | F |
| 560 | A | C | A | F |
| 561 | A | C | A | F |
| 562 | A | C | A | F |
| 563 | A | C | B | F |
| 564 | A | C | A | F |
| 565 | A | C | B | F |
| 566 | A | C | B | E |
| 567 | A | C | B | E |
| 568 | A | C | A | F |
| 569 | A | C | A | F |
| 570 | A | C | B | E |
| 571 | A | C | A | F |
| 572 | A | D | A | F |
| 573 | A | C | A | E |
| 574 | A | C | B | E |
| 575 | A | D | B | F |
| 576 | A | C | A | F |
| 577 | A | C | A | E |
| 578 | A | C | A | E |
| 579 | A | C | A | F |
| 580 | A | C | B | F |
| 581 | A | C | A | F |
| 582 | A | C | A | F |
| 583 | A | C | A | F |
| 584 | A | C | B | F |
| 585 | B | D | A | F |
| 586 | A | C | B | F |
| 587 | A | C | B | F |
| 588 | A | E | B | F |
| 589 | A | E | B | F |
| 590 | B | E | B | F |
| 591 | B | E | A | F |
| 592 | B | E | B | F |
| 593 | B | E | B | F |
| 594 | A | C | A | E |
| 595 | A | D | B | F |
| 596 | B | C | B | F |
| 597 | B | E | B | F |
| 598 | A | D | B | E |
| 599 | A | E | B | F |
| 600 | A | D | B | F |
| 601 | B | E | B | E |
| 602 | A | E | B | F |
| 603 | A | D | B | F |
| 604 | A | C | B | F |
| 605 | A | E | B | F |
| 606 | B | E | B | F |
| 607 | B | E | B | F |
| 608 | A | D | B | F |
| 609 | A | E | B | F |
| 610 | A | D | B | E |
| 611 | A | C | B | F |
| 612 | A | D | B | F |
| 613 | A | E | B | F |
| 614 | B | D | B | F |
| 615 | A | C | A | F |
| 616 | B | E | B | F |
| 617 | A | E | A | F |
| 618 | A | D | B | F |
| 619 | A | E | B | F |
| 620 | A | D | A | E |
| 621 | A | C | B | F |
| 622 | A | D | B | E |
| 623 | A | D | A | F |
| 624 | A | C | A | F |
| 625 | A | C | B | F |
| 626 | A | C | B | F |
| 627 | A | C | B | E |
| 628 | A | D | B | F |
| 629 | A | C | B | F |
| 630 | B | C | B | F |
| 631 | A | C | B | F |
| 632 | A | D | B | F |
| 633 | A | D | A | F |
| 634 | A | C | B | E |
| 635 | A | C | B | F |
| 636 | B | C | B | F |
| 637 | A | D | A | F |
| 638 | A | C | A | F |
| 639 | A | C | B | E |
| 640 | A | C | B | F |
| 641 | A | C | B | F |
| 642 | B | C | A | E |
| 643 | A | D | B | F |
| 644 | A | D | B | F |
| 645 | A | D | B | F |
| 646 | A | C | B | F |
| 647 | A | D | B | F |
| 648 | A | D | A | F |
| 649 | A | D | A | E |
| 650 | A | D | A | F |
| 651 | A | D | A | E |
| 652 | A | D | A | F |
| 653 | A | D | B | F |
| 654 | A | D | A | F |
| 655 | A | E | B | F |
| 656 | A | E | B | F |
| 657 | A | D | B | F |
| 658 | B | D | B | F |
| 659 | A | E | B | F |
| 660 | A | D | A | E |
| 661 | A | E | B | F |
| 662 | A | E | B | F |
| 663 | A | E | A | F |
| 664 | A | E | B | E |
| 665 | A | E | B | D |
| 666 | A | E | B | F |
| 667 | A | E | A | F |
| 668 | A | E | B | F |
| 669 | B | E | B | F |
| 670 | A | E | B | F |
| 671 | A | E | B | F |
| 672 | A | E | A | F |
| 673 | A | E | A | F |
| 674 | A | E | B | F |
| 675 | A | E | A | F |
| 676 | A | E | B | F |
| 677 | A | E | B | |
| 678 | B | E | A | F |
| 679 | A | D | B | F |
| 680 | A | C | B | E |
| 681 | A | C | A | D |
| 682 | A | C | A | F |
| 683 | A | C | B | F |
| 684 | A | E | A | F |
| 6785 | A | C | A | C |
| 686 | A | C | A | F |
| 687 | A | D | A | F |
| 688 | A | C | A | F |
| 689 | A | C | B | F |
| 690 | B | E | B | F |
| 691 | A | E | B | F |
| 692 | B | E | B | F |
| 693 | A | E | B | F |
| 694 | B | E | B | F |
| 695 | A | E | A | F |
| 696 | A | E | B | F |
| 697 | A | D | B | E |
| 698 | A | F | B | F |
| 699 | B | F | B | F |
| 700 | A | E | A | F |
| 701 | B | E | B | F |
| 702 | B | E | B | F |
| 703 | A | D | B | F |
| 704 | A | C | B | F |
| 705 | A | C | A | F |
| 706 | A | D | B | F |
| 707 | A | D | B | F |
| 708 | A | D | B | F |
| 709 | A | D | B | F |
| 710 | A | C | A | F |
| 711 | A | E | B | F |
| 712 | A | D | A | F |
| 713 | A | D | A | F |
| 714 | A | E | A | F |
| 715 | A | D | A | E |
| 716 | A | E | B | F |
| 717 | A | D | B | F |
| 718 | A | E | B | F |
| 719 | B | E | B | F |
| 720 | A | C | A | E |
| 721 | B | D | B | F |
| 722 | B | D | A | F |
| 723 | A | C | B | F |
| 724 | A | C | B | F |
| 725 | A | C | B | F |
| 726 | A | C | B | F |
| 727 | A | C | B | F |
| 728 | A | C | B | F |
| 729 | A | C | A | E |
| 730 | A | C | A | E |
| 731 | A | C | A | E |
| 732 | A | C | A | F |
| 733 | A | C | B | F |
| 734 | A | C | B | F |
| 735 | A | C | A | E |

A = <25 nM,
B = ≥25 nM,
C = 0-10% remaining,
D = >10- 25% remaining,
E = >25-75% remaining,
F = >75-100% remaining

### Example 737

Z-138 Proliferation Assay

Reagents

Z-138 Cells (Catalog #CRL-3001, ATCC, USA)

Iscove's Modified Dulbeco Medium (IMDM) (Catalog #30-2005, Thermo Fisher Scientific, USA)

Fetal Bovine Serum (Catalog #1614007, Thermo Fisher Scientific, USA)

Penicillin-Streptomycin (10,000 U/mL) (Catalog #15140148, Thermo Fisher Scientific, USA)

Cell Titer-Glo (Catalog #G7572, Promega, Corporation, USA)

DMSO (Catalog #D2650, Sigma-Aldrich Corporation, USA)

Z-138 Cell Culture Media

Z-138 cells were cultured in IMDM Medium supplemented with 10% heat in-activated fetal bovine serum and 1% Penicillin-Streptomycin.

Compound Preparation

10 mM stocks of compound were made from fresh powder. DMSO was added to solid compound so that the final concentration was 10 mM. Compounds were vortexed and allowed to sit for 5 minutes to ensure complete solubility. Solvated compound was aliquoted into 1 ml Eppendorf tubes and stored in a −20° C. freezer.

Z-138 Cell Culture

Z-138 cells were maintained in culture using standard cell culturing techniques.

Cells were maintained at a density of 0.8×10⁶ cells/ml.

Cell Titer Glo Reagent

Re-constitute cell titer-glo reagent according to manufacturer's protocol.

Z-138 Proliferation Assay

Z-138 cells were removed from culture and cell viability and density were determined using a CellDrop™ Automated Cell Counter with Tryphan blue stain according to manufacturer's protocols. Cells were centrifuged and resuspended using fresh media to a cell density of 0.1×10⁶ cells/ml. Did not use cells unless cell viability was greater than 90%. 40 μl of cell suspension was added to the wells of a 384 well tissue culture plate (4,000 cells/well final). Upon completing the addition of the cells to the plate, the plates were centrifuged for 30 seconds at 300 rpms to help the cells settle and to bring air bubbles to the top of the wells. Air bubbles were popped using a pipette tip.

10-point dose response curves were performed using a D300e Digital Dispenser. The highest concentration of compound used in the assay was 10,000 nM. 1:2 dilutions were performed over a 10-point range with the final DMSO concentration being 0.1%. The cells were cultured for 72 hours in the presence of compound.

After 72 hours, the cell plates were removed from the incubator and allowed to equilibrate to room temperature (about 15 minutes). 20 μl of cell titer-glo reagent was added to each well of the 384 well plate. Incubated the plate for 30 minutes in the dark. After 30 minutes, ensured that there were no air bubbles in the plate (popped bubbles with pipette tip if necessary) and analyzed plate using a luminometer.

EC₅₀ curves were generated using Prism Graph Pad. In brief, POCs (percent of control, compound/DMSO control×100) were plotted against the compound concentrations (log scale). EC₅₀ curves were generated using a 4-parameter curve fit.

Results

The EC₅₀ and Yₘᵢₙ for compounds provided herein in the above proliferation assay are provided below.

| Example | EC₅₀ (nM) | Yₘᵢₙ (%) | Example | EC₅₀ (nM) | Yₘᵢₙ (%) |
|---|---|---|---|---|---|
| 7 | A | D | 379 | A | D |
| 12 | A | D | 421 | B | F |
| 14 | A | D | 422 | B | D |
| 17 | A | D | 488 | B | E |
| 19 | A | D | 518 | B | D |
| 25 | A | D | 519 | B | E |
| 32 | A | D | 546 | B | F |
| 34 | B | E | 550 | B | D |
| 36 | B | E | 553 | B | D |
| 37 | B | F | 588 | B | F |
| 39 | C | D | 594 | A | D |
| 40 | A | D | 606 | B | F |
| 41 | B | D | 614 | B | F |
| 43 | C | F | 615 | B | E |
| 46 | A | D | 619 | B | F |
| 48 | A | D | 621 | A | D |
| 51 | B | D | 622 | B | E |
| 52 | A | D | 692 | C | E |
| 64 | A | E | 703 | B | E |
| 72 | B | E | 705 | B | E |
| 184 | B | D | 710 | A | E |
| 212 | C | D | 717 | B | E |
| 224 | A | E | 726 | A | D |
| 269 | A | D | 728 | A | E |
| 287 | A | D | 729 | A | D |
| 319 | B | D | 730 | A | D |
| 331 | B | D | 731 | A | D |
| 335 | B | D | 732 | A | D |
| 339 | B | D | 733 | A | D |

A = <10 nM,
B = 10-<100 nM,
C = 100-500 nM,
D = 0-10% remaining,
E = >10-25% remaining,
F = >25-50% remaining

This disclosure is not to be limited in scope by the embodiments disclosed in the examples which are intended as single illustrations of individual aspects, and any equivalents are within the scope of this disclosure. Various modifications in addition to those shown and described herein will become apparent to those skilled in the art from the foregoing description. Such modifications are intended to fall within the scope of the appended claims.

Various references such as patents, patent applications, and publications are cited herein, the disclosures of which are hereby incorporated by reference herein in their entireties.

What is claimed is:

1. A compound of Formula I or II: 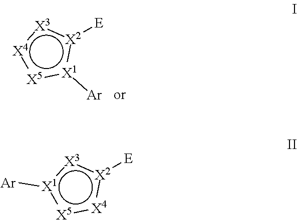
or a pharmaceutically acceptable derivative thereof, wherein:
Ar is aryl, heteroaryl, C₅₋₇cycloalkyl, C₅₋₇cycloalkenyl, a 5-7 membered heterocyclyl, or a 5-7 membered heterocycloalkenyl;
E is a moiety that binds to an E3 ubiquitin ligase;
X¹-X² are each independently N or C; and
X³-X⁵ are each independently CR, N, NR, S or O;
where each R is independently H, alkyl, alkenyl, alkynyl, cycloalkyl or heterocyclyl; or two R groups that are on adjacent positions on the ring together form alkylene; or R and Ar that are on adjacent positions on the 5 membered ring together form a fused ring;
with the provisos:
i) in Formula II, when X¹ is C, X², X⁴ and X⁵ are N and X³ is CH, or when X¹ and X² are C, X³ is NMe, X⁴ is N and X⁵ is CH, then E is not an isoindolindione moiety;
ii) in Formula II, when X¹ and X² are C, X³ is CH, X⁴ is N and X⁵ is NMe, then Ar is not 5-fluoro-2-pyridyl;
iii) in Formula II, when X¹ is N, X² is C, X³ and X⁴ are CH and X⁵ is N, or when X¹ and X³ are N, X² is C, and X⁴ and X⁵ are CH, or when then Ar is not phenyl;
iv) in Formula II, the ring containing X¹-X⁵ is not 1,2,3-triazol-1,4-diyl;
v) Ar is not tetrahydropyran-2-yl; and
vi) the compound is not 3-[1,3-dihydro-1-oxo-5-(5-phenyl-4-oxazolyl)-2H-isoindol-2-yl]-2,6-piperidinedione, 3-[1,3-dihydro-1-oxo-5-(2-phenyl-4-oxazolyl)-2H-isoindol-2-yl]-2,6-piperidinedione, or 3-[1,3-dihydro-1-oxo-5-(3-phenyl-1H-1,2,4-triazol-5-yl)-2H-isoindol-2-yl]-2,6-piperidinedione.

2. The compound of claim 1, wherein X¹ is C and X² is N.

3. The compound of claim 1, wherein X¹ is N and X² is C.

4. The compound of claim 1, wherein the compound has the structure:

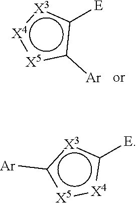

5. The compound of claim 1 having one of the following formulae:

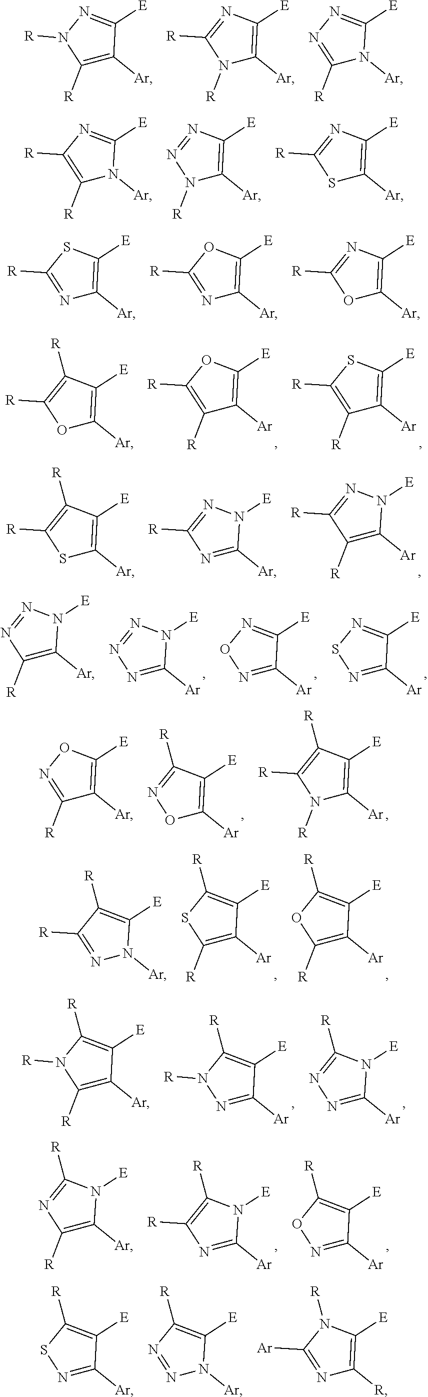

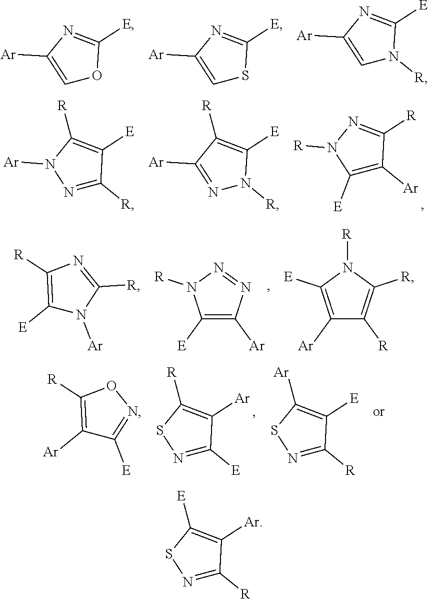

6. The compound of claim 1 having the structure:

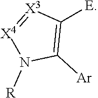

7. The compound of claim 1 having the structure:

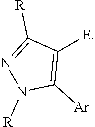

8. The compound of claim 1 having the structure:

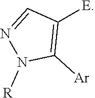

9. The compound of claim 1 having the structure:

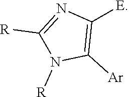

10. The compound of claim 1 having the structure:

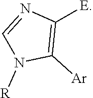

11. The compound of claim 1 having the structure:

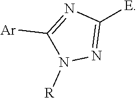

12. The compound of claim 1 having the structure:

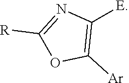

13. The compound of claim 1 having the structure:

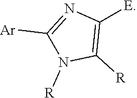

14. The compound of claim 1 having the structure:

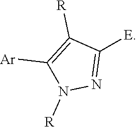

15. The compound of claim 1 having the structure:

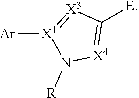

16. The compound of claim 1, wherein each R is independently H, alkyl, alkenyl, alkynyl, cycloalkyl or heterocyclyl; or two R groups that are on adjacent positions on the ring together form alkylene.

17. The compound of claim 1, wherein each R is independently H, alkyl, alkenyl or alkynyl.

18. The compound of claim 1, wherein each R is independently H, alkyl, cycloalkyl, heterocyclyl or aryl; or two R groups that are on adjacent positions on the ring together form lower alkylene.

19. The compound of claim 1, wherein each R is independently H or alkyl; or two R groups that are on adjacent positions on the ring together form lower alkylene.

20. The compound of claim 1, wherein each R is independently H, alkyl or haloalkyl.

21. The compound of claim 1, wherein E is a moiety that binds to cereblon.

22. The compound of claim 1, wherein E contains a thalidomide, lenalidomide or pomalidomide moiety, or an analog or derivative thereof.

23. The compound of claim 1, wherein E has one of the following formulae: 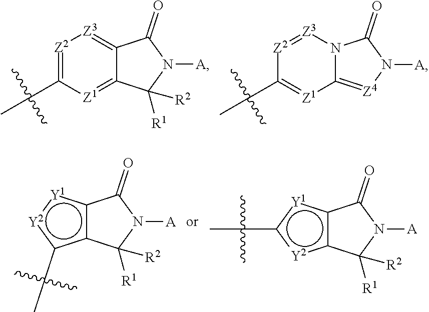
wherein:
A is a cyclic amide or cyclic imide or a derivative thereof;
R¹ and R² are each independently H, alkyl, alkenyl, alkynyl, cycloalkyl, heterocyclyl, aryl or heteroaryl;
one of Y¹ and Y² is S and the other is CR³, where R³ is H, alkyl, alkenyl, alkynyl, cycloalkyl, heterocyclyl, aryl or heteroaryl; and
Z¹-Z⁴ are each independently N or CR⁴, where each R⁴ is independently H, alkyl, alkenyl, alkynyl, cycloalkyl, heterocyclyl, aryl or heteroaryl.

24. The compound of claim 23, wherein A is a cyclic imide having the structure: 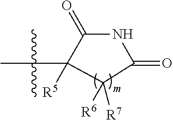
wherein: R⁵ is H or alkyl; R⁶ and R⁷ are each independently H, alkyl, alkenyl, alkynyl, cycloalkyl or heterocyclyl; and m is an integer from 1-4.

25. The compound of claim 23, wherein A has the structure:

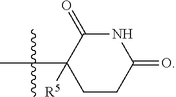

26. The compound of claim 25, wherein R⁵ is H.

27. The compound of claim 23, wherein R¹ and R² are each H.

28. The compound of claim 23, wherein R³ is H.

29. The compound of claim 23, wherein R⁴ is H.

30. The compound of claim 23, wherein Y¹ is S and Y² is CH.

31. The compound of claim 23, wherein Y¹ is CH and Y² is S.

32. The compound of claim 1, wherein E has the structure: 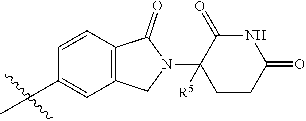
wherein: R⁵ is H or alkyl.

33. The compound of claim 32, wherein R⁵ is H.

34. The compound of claim 1, wherein Ar is optionally substituted phenyl, optionally substituted biphenyl, optionally substituted naphthyl, optionally substituted pyridyl, optionally substituted pyrimidinyl, optionally substituted pyridazinyl, optionally substituted pyrazolyl, optionally substituted pyridopyrazolyl, optionally substituted isoxazolyl, optionally substituted indolyl, optionally substituted isoindolyl, optionally substituted thienyl, optionally substituted benzofuryl, optionally substituted imidazopyridyl, optionally substituted benzopyrazolyl, optionally substituted pyrrolopyridyl, optionally substituted benzimidazolyl, optionally substituted benzothiazolyl, optionally substituted thienopyridyl, optionally substituted dihydrobenzofuryl, optionally substituted benzopyridazinyl, optionally substituted benzopyranyl, optionally substituted benzothienyl, optionally substituted triazolopyrimidinyl, optionally substituted piperidinyl, optionally substituted cyclohexenyl, optionally substituted tetrahydropyridyl, optionally substituted tetrahydrofuranyl, optionally substituted dihydrofuranyl, optionally substituted morpholinyl, optionally substituted tetrahydroisoquinolinyl, optionally substituted azepinyl, optionally substituted isoquinolinyl, optionally substitutedcycloheptenyl, optionally substituted indenyl, optionally substituted dihydronaphthyl, optionally substituted 8-azabicyclooctanyl, optionally substituted adamantanyl, optionally substituted dihydroindenyl, optionally substituted cyclopropyl or optionally substituted cyclohexyl.

35. The compound of claim 1, wherein Ar is phenyl, biphenyl, naphthyl, pyridinyl, pyrimidinyl, pyrazolyl, pyridopyrazolyl, isoxazolyl, indolyl, isoindolyl, thienyl, dihydrobenzofuryl, dihydroindenyl, cyclopropyl or cyclohexyl, each optionally substituted with one or more substituents each independently selected from halo, cyano, alkyl, alkenyl, alkynyl, haloalkyl, cycloalkyl, heterocyclyl, heteroaryl, cycloalkylalkyl, heterocyclylalkyl, COR⁸, OR⁹, NR¹⁰R¹¹ and S(O)ₙR¹², where:
R⁸ is alkyl, OR¹³ or NR¹⁴R¹⁵;
R⁹ is H, alkyl, cycloalkyl, heterocyclyl, aryl, heteroaryl or COR¹⁶;
R¹⁰ and R¹¹ are each independently H, alkyl, cycloalkyl, heterocyclyl, aryl, heteroaryl or COR¹⁷;
R¹² is alkyl, cycloalkyl, heterocyclyl, aryl, heteroaryl, OR¹⁸ or NR¹⁴R¹⁵;
each R¹³, R¹⁴ and R¹⁵ are each independently H, alkyl, cycloalkyl, heterocyclyl, aryl or heteroaryl;
R¹⁶ is alkyl, cycloalkyl, heterocyclyl, aryl, heteroaryl, OR¹³ or NR¹⁴R¹⁵;
R¹⁷ is alkyl, cycloalkyl, heterocyclyl, aryl, heteroaryl, OR¹³ or NR¹⁴R¹⁵;
R¹⁸ is alkyl, cycloalkyl, heterocyclyl, aryl or heteroaryl; and
n is 0, 1 or 2.

36. The compound of claim 1, wherein Ar is phenyl, optionally substituted with one or more substituents each independently selected from halo, cyano, alkyl, alkenyl, alkynyl, haloalkyl, cycloalkyl, heterocyclyl, aryl, heteroaryl, cycloalkylalkyl, heterocyclylalkyl, COR⁸, OR⁹, NR¹⁰R¹¹ and S(O)ₙR¹².

37. The compound of claim 1, wherein Ar is phenyl, optionally substituted with one or more substituents each independently selected from chloro, fluoro, cyano, methyl, isopropyl, isobutyl, tert-butyl, trifluoromethyl, difluoromethyl, hydroxymethyl, methoxymethyl, phenoxymethyl, dimethylaminomethyl, cyclopropyl, 1-cyano-1-cyclopropyl, 1-piperidinyl, 1-pyrrolidinylmethyl, morpholin-4-yl, 4-methylpiperazin-1-yl, 4-methylpiperazin-1-ylmethyl, 4-tert-butyloxycarbonyl-1-piperazinyl, morpholin-4-ylmethyl, 1-pyrrolidinyl, phenyl, 4-cyanophenyl, 4-hydroxyphenyl, 1-pyrazolyl, 2-pyridinyl, 3-pyridinyl, 4-pyridinyl, hydroxy, methoxy, difluoromethoxy, trifluoromethoxy, benzyloxy, (3-methoxybenzyl)oxy, 3-pyridinyloxy, 3-(4-morpholinyl)propoxy, CONH₂, CONHMe, CONMe₂, CONH-cyclopentyl, CONH-cyclohexyl, CONH-benzyl, CO-(4-morpholinyl), CO-(4-methylpiperazin-1-yl), NH₂, NMe₂, NHCOPh, SO₂Me, SO₂NH-cyclohexyl and SO₂-(1-pyrrolidinyl).

38. The compound of claim 1, wherein Ar is phenyl, optionally substituted with one or more substituents each independently selected from chloro, fluoro, cyano, methyl, ethyl, isobutyl, tert-butyl, difluoromethyl, trifluoromethyl, hydroxymethyl, methoxymethyl, dimethylaminomethyl, cyclopropyl, 1-cyano-1-cyclopropyl, 1-pyrrolidinyl, 1-pyrazolyl, 3-pyridinyl, hydroxy, methoxy, CONH₂, CONHMe, CONMe₂, NH₂ and NMe₂.

39. The compound of claim 1, wherein Ar is unsubstituted phenyl, unsubstituted 4-biphenyl or unsubstituted 1-naphthyl.

40. The compound of claim 1, wherein Ar is unsubstituted phenyl.

41. The compound of claim 1, wherein Ar is pyrazolyl, optionally substituted with one or more substituents each independently selected from halo, cyano, alkyl, alkenyl, alkynyl, haloalkyl, cycloalkyl, heterocyclyl, aryl, heteroaryl, cycloalkylalkyl, heterocyclylalkyl, COR⁸, OR⁹, NR¹⁰R¹¹ and S(O)ₙR¹².

42. The compound of claim 1, wherein Ar is pyrazolyl, optionally substituted with one or more substituents each independently selected from chloro, fluoro, cyano, methyl, isopropyl, isobutyl, tert-butyl, trifluoromethyl, difluoromethyl, hydroxymethyl, methoxymethyl, phenoxymethyl, dimethylaminomethyl, cyclopropyl, 1-cyano-1-cyclopropyl, 1-piperidinyl, 1-pyrrolidinylmethyl, morpholin-4-yl, 4-methylpiperazin-1-yl, 4-methylpiperazin-1-ylmethyl, 4-tert-butyloxycarbonyl-1-piperazinyl, morpholin-4-ylmethyl, 1-pyrrolidinyl, phenyl, 4-cyanophenyl, 4-hydroxyphenyl, 1-pyrazolyl, 2-pyridinyl, 3-pyridinyl, 4-pyridinyl, hydroxy, methoxy, benzyloxy, 3-methoxybenzyloxy, 3-pyridinyloxy, 3-(4-morpholinyl)propoxy, CONH₂, CONHMe, CONMe₂, CONH-cyclopentyl, CONH-cyclohexyl, CONH-benzyl, CO-(4-morpholinyl), CO-(4-methylpiperazin-1-yl), NH₂, NMe₂, NHCOPh, SO₂Me, SO₂NH-cyclohexyl and SO₂-(1-pyrrolidinyl).

43. The compound of claim 1 having the structure: 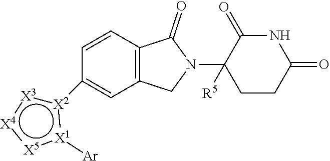
wherein R⁵ is H or alkyl.

44. The compound of claim 1 having the structure: 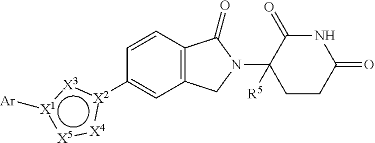
wherein R⁵ is H or alkyl.

45. The compound of claim 1 having the structure: 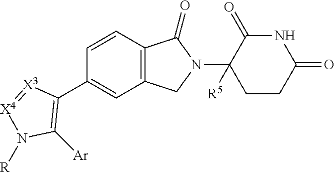
wherein R⁵ is H or alkyl.

46. The compound of claim 1 having the structure: 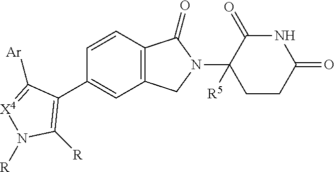
wherein R⁵ is H or alkyl.

47. The compound of claim 1 having the structure: 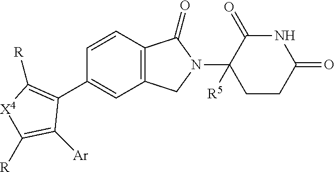
wherein R⁵ is H or alkyl.

48. The compound of claim 1 having the structure: 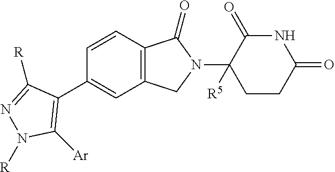
wherein R⁵ is H or alkyl.

49. The compound of claim 1 having the structure:

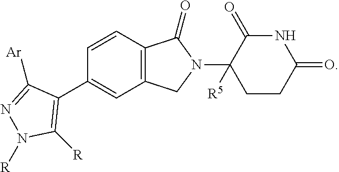

50. The compound of claim 1 having the structure:

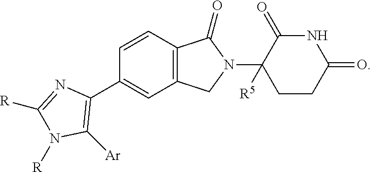

51. The compound of claim 1 having the structure:

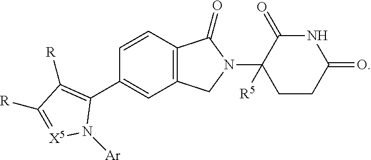

52. The compound of claim 1 having the structure:

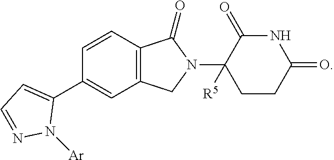

53. The compound of claim 1 having the structure:

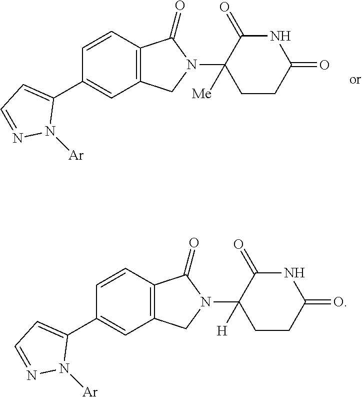

54. The compound of claim 1 having the structure:

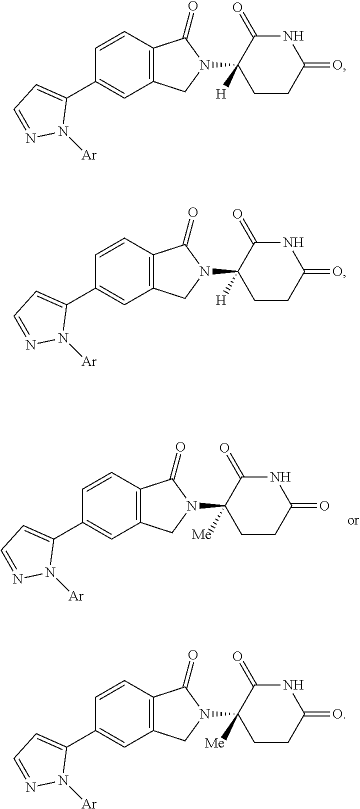

55. The compound of claim 1 having the structure:

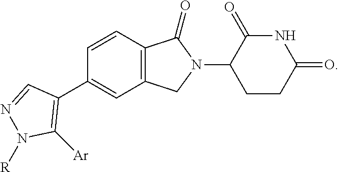

56. The compound of claim 1 having the structure:

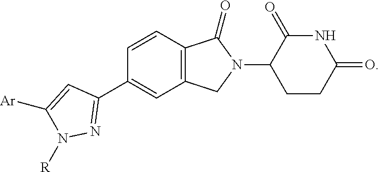

57. The compound of claim 1 having the structure:

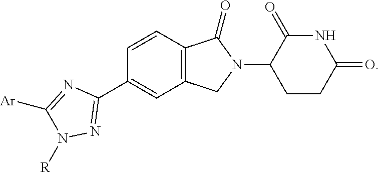

58. The compound of claim 1 having the structure:

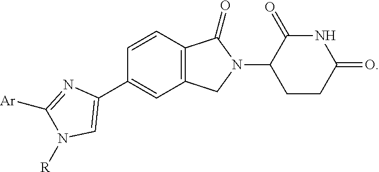

59. A pharmaceutical composition, comprising the compound of claim 1 and a pharmaceutically acceptable carrier.

60. A method of treating a subject having a proliferative disease, comprising administering to the subject the compound of claim 1.

61. A method of treating a subject having cancer, comprising administering to the subject the compound of claim 1.

62. The method of claim 61, wherein the cancer is acute myeloid leukemia (AML), myelodysplastic syndrome, (MDS) (including 5q-MDS), colon cancer, acute lymphoblastic leukemia (ALL), chronic lymphocytic leukemia (CLL), chronic myeloid leukemia (CML), B-cell lymphoma or mantle cell lymphoma (MCL).

63. The method of claim 61, wherein the cancer is a BTK inhibitor resistant cancer.

64. A method of treating AML, glioma, thyroid cancer, lung cancer, colorectal cancer, head and neck cancer, stomach cancer, liver cancer, pancreatic cancer, renal cancer, urothelial cancer, prostate cancer, testis cancer, breast cancer, cervical cancer, endometrial cancer, ovarian cancer, melanoma, multiple myeloma, hepatocellular carcinoma or gastric cancer in a subject, comprising administering to the subject the compound of claim 1.

65. A method of treating a subject having an autoimmune disorder, comprising administering to the subject the compound of claim 1.
